SEQUENCE LISTING (1220 sequences)

SEQ ID NO: 1 - DNA, 141 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 3 - DNA, 1923 bp, Artificial Sequence
  Description of Artificial Sequence: Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 4 - DNA, 1272 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 5 - DNA, 547 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 6 - DNA, 1179 bp, Artificial Sequence
  Description of Artificial Sequence: Synthetic polynucleotide
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 7 - DNA, 8 bp, Artificial Sequence
  Description of Artificial Sequence: Synthetic oligonucleotide
gtttaaac

SEQ ID NO: 8 - DNA, 581 bp, Artificial Sequence
  Description of Artificial Sequence: Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtc

SEQ ID NO: 9 - DNA, 225 bp, Artificial Sequence
  Description of Artificial Sequence: Synthetic polynucleotide
tgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggc

SEQ ID NO: 10 - DNA, 213 bp, Artificial Sequence
  Description of Artificial Sequence: Synthetic polynucleotide
taagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggta

SEQ ID NO: 11 - DNA, 1386 bp, Artificial Sequence
  Description of Artificial Sequence: Synthetic polynucleotide
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggcagggtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgcgcagcaccaagttcaccatctacaacaacatgttctgcgccggcttccacgagggcggcagggacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaa

SEQ ID NO: 12 - DNA, 1386 bp, Artificial Sequence
  Description of Artificial Sequence: Synthetic polynucleotide
atgcagcgcgtgaacatgatcatggccgagagccccggcctgatcaccatctgcctgctgggctacctgctgagcgccgagtgcaccgtgttcctggaccacgagaacgccaacaagatcctgaaccgccccaagcgctacaacagcggcaagctggaggagttcgtgcagggcaacctggagcgcgagtgcatggaggagaagtgcagcttcgaggaggcccgcgaggtgttcgagaacaccgagcgcaccaccgagttctggaagcagtacgtggacggcgaccagtgcgagagcaacccctgcctgaacggcggcagctgcaaggacgacatcaacagctacgagtgctggtgccccttcggcttcgagggcaagaactgcgagctggacgtgacctgcaacatcaagaacggccgctgcgagcagttctgcaagaacagcgccgacaacaaggtggtgtgcagctgcaccgagggctaccgcctggccgagaaccagaagagctgcgagcccgccgtgcccttcccctgcggcagggtgagcgtgagccagaccagcaagctgacccgcgccgaggccgtgttccccgacgtggactacgtgaacagcaccgaggccgagaccatcctggacaacatcacccagagcacccagagcttcaacgacttcacccgcgtggtgggcggcgaggacgccaagcccggccagttcccctggcaggtggtgctgaacggcaaggtggacgccttctgcggcggcagcatcgtgaacgagaagtggatcgtgaccgccgcccactgcgtggagaccggcgtgaagatcaccgtggtggccggcgagcacaacatcgaggagaccgagcacaccgagcagaagcgcaacgtgatccgcatcatcccccaccacaactacaacgccgccatcaacaagtacaaccacgacatcgccctgctggagctggacgagcccctggtgctgaacagctacgtgacccccatctgcatcgccgacaaggagtacaccaacatcttcctgaagttcggcagcggctacgtgagcggctggggccgcgtgttccacaagggccgcagcgccctggtgctgcagtacctgcgcgtgcccctggtggaccgcgccacctgcctgctgagcaccaagttcaccatctacaacaacatgttctgcgccggcttccacgagggcggcagggacagctgccagggcgacagcggcggcccccacgtgaccgaggtggagggcaccagcttcctgaccggcatcatcagctggggcgaggagtgcgccatgaagggcaagtacggcatctacaccaaggtgagccgctacgtgaactggatcaaggagaagaccaagctgacctaa

SEQ ID NO: 13 - DNA, 1876 bp, Artificial Sequence
  Description of Artificial Sequence: Synthetic polynucleotide
cgcagccaccatggcggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 1194 bp, Artificial Sequence
  Description of Artificial Sequence: Synthetic polynucleotide
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagaccgctaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 141 bp, Artificial Sequence
  Description of Artificial Sequence: Synthetic polynucleotide
aataaacgataacgccgttggtggcgtgaggcatgtaaaaggttacatcattatcttgttcgccatccggttggtataaatagacgttcatgttggtttttgtttcagttgcaagttggctgcggcgcgcgcagcaccttt

SEQ ID NO: 16 - DNA, 556 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactga

SEQ ID NO: 17 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc

SEQ ID NO: 18 - DNA, 241 bp, Artificial Sequence
  Description of Artificial Sequence: Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 19 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcgtataagaactgtatgagaccac

SEQ ID NO: 20 - DNA, 150 bp, Artificial Sequence
  Description of Artificial Sequence: Synthetic polynucleotide
ataaacgataacgccgttggtggcgtgaggcatgtaaaaggttacatcattatcttgttcgccatccggttggtataaatagacgttcatgttggtttttgtttcagttgcaagttggctgcggcgcgcgcagcacctttgcggccatct

SEQ ID NO: 21-38 - skipped (18 entries)

SEQ ID NO: 39 - DNA, 16 bp, Artificial Sequence
  Description of Artificial Sequence: Synthetic oligonucleotide
gcgcgctcgctcgctc

SEQ ID NO: 40-50 - skipped (11 entries)

SEQ ID NO: 51 - DNA, 141 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 52 - DNA, 130 bp, Artificial Sequence
  Description of Artificial Sequence: Synthetic polynucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 53 - DNA, 3123 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 54 - DNA, 3117 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 55 - DNA, 2841 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 56 - DNA, 2835 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaaggccgccaccatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 57 - DNA, 3912 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 58 - DNA, 3906 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 59 - DNA, 3630 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 60 - DNA, 3624 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgccgccaccatgatcatggccgagagccctggcctgatcaccatctgcctgctgggctacctgctgagcgccgagtgcaccgtgttcctggaccacgagaacgccaacaagatcctgaaccggcccaagagatacaacagcggcaagctggaggagttcgtgcagggcaacctggagagggagtgcatggaggagaagtgcagcttcgaggaggccagggaagtgttcgagaacaccgagcggaccaccgagttctggaagcagtacgtggacggcgaccagtgcgagagcaacccttgcctgaacggcggcagctgcaaggacgacatcaacagctacgagtgctggtgccctttcggcttcgagggcaagaactgcgagctggacgtgacctgcaacatcaagaacggccgctgcgagcagttctgcaagaacagcgccgacaacaaagtggtgtgtagctgcaccgagggctacagactggccgagaaccagaagagctgcgagcccgccgtgcccttcccctgcggcagagtgagcgtgtcccagaccagcaagctgaccagagccgagaccgtgttccccgacgtggactacgtgaatagcaccgaggccgagaccatcctggacaacatcacccagagcacccagtccttcaacgacttcaccagagttgtgggcggcgaggacgccaagcccggccagttcccctggcaggtggtgctgaacggcaaagtggatgccttctgcggcggcagcatcgtgaacgagaagtggatcgtgacagccgcccactgcgtggagaccggcgtgaagatcaccgtggtggccggcgaacacaatatcgaggagaccgagcacaccgagcagaagcggaacgtcatccggattatcccccaccacaactacaacgccgccatcaacaagtacaaccacgacatcgccctgctggagctggacgagcctctggtgctgaatagctacgtgacccccatctgcatcgccgacaaggagtacaccaacatcttcctgaagttcggcagcggctacgtgtccggctggggcagagtgttccacaagggcagaagcgccctggtgctgcagtacctgagagtgcccctggtggacagagccacctgcctgttgagcaccaagttcaccatctacaacaacatgttctgcgccggcttccacgagggcggcagagacagctgccagggcgacagcggcggaccccacgtgaccgaagtggagggcaccagcttcctgaccggcatcatcagctggggcgaggagtgcgccatgaagggcaagtacggcatctacaccaaagtgagccggtacgtgaactggatcaaggagaaaaccaagctgacctgagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcaggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 61 - DNA, 4257 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcggccgcacgcgtagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataagtgcggaccgagcggccgctctagagcatggctacgtagataagtagcatggcgggttaatcattaactacacctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 62 - DNA, 2510 bp, Artificial Sequence
  Description of Artificial Sequence: Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagcggccgcgctagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacactcgagggccctgtctcctcagcttcaggcaccaccactgacctgggacagtgaatccggacatcgattctaaggtaaatataaaatttttaagtgtataatttgttaaactactgattctaattgtttctctcttttagattccaacctttggaactgaattctagaccaccatgcagagggtgaacatgatcatggctgagagccctggcctgatcaccatctgcctgctgggctacctgctgtctgctgagtgcactgtgttcctggaccatgagaatgccaacaagatcctgaacaggcccaagagatacaactctggcaagctggaggagtttgtgcagggcaacctggagagggagtgcatggaggagaagtgcagctttgaggaggccagggaggtgtttgagaacactgagaggaccactgagttctggaagcagtatgtggatggggaccagtgtgagagcaacccctgcctgaatgggggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcactgagggctacaggctggctgagaaccagaagagctgtgagcctgctgtgccattcccatgtggcagagtgtctgtgagccagaccagcaagctgaccagggctgaggctgtgttccctgatgtggactatgtgaacagcactgaggctgaaaccatcctggacaacatcacccagagcacccagagcttcaatgacttcaccagggtggtggggggggaggatgccaagcctggccagttcccctggcaagtggtgctgaatggcaaggtggatgccttctgtgggggcagcattgtgaatgagaagtggattgtgactgctgcccactgtgtggagactggggtgaagatcactgtggtggctggggagcacaacattgaggagactgagcacactgagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagggtgttccacaagggcaggtctgccctggtgctgcagtacctgagggtgcccctggtggacagggccacctgcctgaggagcaccaagttcaccatctacaacaacatgttctgtgctggcttccatgaggggggcagggacagctgccagggggactctgggggcccccatgtgactgaggtggagggcaccagcttcctgactggcatcatcagctggggggaggagtgtgccatgaagggcaagtatggcatctacaccaaagtctccagatatgtgaactggatcaaggagaagaccaagctgacctaatgactccatggttcgaatgctttatttgtgaaatttgtgatgctattgctttatttgtaaccattataagctgcaataaacaagttaacaacaacaattgcattcattttatgtttcaggttcagggggaggtgtgggaggttttttaaaactagtgcggccgctctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 63 - DNA, 126 bp, Artificial Sequence
  Description of Artificial Sequence: Synthetic polynucleotide
cctgcaggcagctgcgcgctcgctcgctcactgaggccgcccgggaaacccgggcgtgcccgggcgcctcagtgagcgagcgagcgcgcagagagggagtggccaactccatcactaggggttcct

SEQ ID NO: 64 - DNA, 120 bp, Artificial Sequence
  Description of Artificial Sequence: Synthetic polynucleotide
aggaacccctagtgatggagttggccactccctctctgcgcgctcgctcgctcactgaggccgcccgggaaacccgggcgtgcgcctcagtgagcgagcgagcgcgcagctgcctgcagg

SEQ ID NO: 65 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 66 - DNA, 141 bp, Artificial Sequence; identical to SEQ ID NO: 15
  Description of Artificial Sequence: Synthetic polynucleotide

SEQ ID NO: 67 - DNA, 1876 bp, Artificial Sequence
  Description of Artificial Sequence: Synthetic polynucleotide
cgcagccaccatggcggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 68 - DNA, 129 bp, Artificial Sequence
  Description of Artificial Sequence: Synthetic polynucleotide
atcatggagataattaaaatgataaccatctcgcaaataaataagtattttactgttttcgtaacagttttgtaataaaaaaacctataaatattccggattattcataccgtcccaccatcgggcgcg

SEQ ID NO: 69 - DNA, 1203 bp, Artificial Sequence
  Description of Artificial Sequence: Synthetic polynucleotide
gccgccaccatggagttggtgggctggctcgtggacaaaggcattacttcggaaaagcagtggattcaggaggatcaggcatcttacatctcattcaacgctgccagtaactcgaggtcccagatcaaggcagcgctggacaacgcgggaaagattatgagtctgaccaaaactgctccagactacctcgttggtcagcaaccggtggaagatatctccagcaacaggatctacaagattctggagctcaacggctacgaccctcaatacgctgcctcagtgttcttgggttgggccaccaagaaattcggcaagagaaacactatctggctgttcggccccgctaccactggaaagacaaacatcgcagaagcgattgctcacacggtgccattctacggctgcgtcaactggacaaacgagaacttcccgttcaacgactgtgtcgataagatggttatctggtgggaggaaggaaagatgacggccaaagtggtcgaaagcgccaaggcaattctgggtggctctaaagtgcgcgtcgaccagaagtgcaaatcttcagctcaaatcgatcctacccccgttattgtgacatcaaacacgaacatgtgtgccgtgatcgacggaaacagtacaacgttcgaacaccagcaacctctccaggatcgtatgttcaagttcgagctcacccgccgtttggaccatgatttcggcaaggtcactaaacaagaggttaaggacttcttccgctgggctaaagatcacgttgtggaggttgaacatgagttctacgtcaagaaaggaggtgctaagaaacgtccagccccgtcggacgcagatatctccgaacctaagagggtgagagagtcggtcgcacagccaagcacttctgacgcagaagcttccattaactacgcagataggtaccaaaacaagtgcagcagacacgtgggtatgaacttgatgctgttcccatgccgccagtgtgagcgtatgaaccaaaactctaacatctgtttcacacatggccagaaggactgcctcgaatgtttccctgtgtcagagagtcagcccgtctcagtcgttaagaaagcttaccaaaagttgtgctacatccaccatattatgggtaaagtccctgatgcctgtaccgcttgtgatctggtcaacgtggatttggacgactgtattttcgagcaataa

SEQ ID NO: 70 - DNA, 388 bp, Artificial Sequence
  Description of Artificial Sequence: Synthetic polynucleotide
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacttccatagaag

SEQ ID NO: 71 - DNA, 1662 bp, Artificial Sequence
  Description of Artificial Sequence: Synthetic polynucleotide
gccgccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

SEQ ID NO: 72 - DNA, 581 bp, Artificial Sequence; identical to SEQ ID NO: 8
  Description of Artificial Sequence: Synthetic polynucleotide

SEQ ID NO: 73 - DNA, 225 bp, Artificial Sequence; identical to SEQ ID NO: 9
  Description of Artificial Sequence: Synthetic polynucleotide

SEQ ID NO: 74 - DNA, 1177 bp, Artificial Sequence
  Description of Artificial Sequence: Synthetic polynucleotide
ggctcagaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 75-100 - skipped (26 entries)

SEQ ID NO: 101 - DNA, 70 bp, Artificial Sequence
  Description of Artificial Sequence: Synthetic oligonucleotide
gcgcgctcgctcgctcactgaggccgcccgggaaacccgggcgtgcgcctcagtgagcgagcgagcgcgc

SEQ ID NO: 102 - DNA, 70 bp, Artificial Sequence
  Description of Artificial Sequence: Synthetic oligonucleotide
gcgcgctcgctcgctcactgaggcgcacgcccgggtttcccgggcggcctcagtgagcgagcgagcgcgc

SEQ ID NO: 103 - DNA, 72 bp, Artificial Sequence
  Description of Artificial Sequence: Synthetic oligonucleotide
gcgcgctcgctcgctcactgaggccgtcgggcgacctttggtcgcccggcctcagtgagcgagcgagcgcgc

SEQ ID NO: 104 - DNA, 72 bp, Artificial Sequence
  Description of Artificial Sequence: Synthetic oligonucleotide
gcgcgctcgctcgctcactgaggccgggcgaccaaaggtcgcccgacggcctcagtgagcgagcgagcgcgc

SEQ ID NO: 105 - DNA, 72 bp, Artificial Sequence
  Description of Artificial Sequence: Synthetic oligonucleotide
gcgcgctcgctcgctcactgaggccgcccgggcaaagcccgggcgtcggcctcagtgagcgagcgagcgcgc

SEQ ID NO: 106 - DNA, 72 bp, Artificial Sequence
  Description of Artificial Sequence: Synthetic oligonucleotide
gcgcgctcgctcgctcactgaggccgacgcccgggctttgcccgggcggcctcagtgagcgagcgagcgcgc

SEQ ID NO: 107 - DNA, 83 bp, Artificial Sequence
  Description of Artificial Sequence: Synthetic oligonucleotide
gcgcgctcgctcgctcactgaggccgcccgggcaaagcccgggcgtcgggctttgcccggcctcagtgagcgagcgagcgcgc

SEQ ID NO: 108 - DNA, 83 bp, Artificial Sequence
  Description of Artificial Sequence: Synthetic oligonucleotide
gcgcgctcgctcgctcactgaggccgggcaaagcccgacgcccgggctttgcccgggcggcctcagtgagcgagcgagcgcgc

SEQ ID NO: 109 - DNA, 77 bp, Artificial Sequence
  Description of Artificial Sequence: Synthetic oligonucleotide
gcgcgctcgctcgctcactgaggccgaaacgtcgggcgacctttggtcgcccggcctcagtgagcgagcgagcgcgc

SEQ ID NO: 110 - DNA, 77 bp, Artificial Sequence
  Description of Artificial Sequence: Synthetic oligonucleotide
gcgcgctcgctcgctcactgaggccgggcgaccaaaggtcgcccgacgtttcggcctcagtgagcgagcgagcgcgc

SEQ ID NO: 111 - DNA, 51 bp, Artificial Sequence
  Description of Artificial Sequence: Synthetic oligonucleotide
gcgcgctcgctcgctcactgaggcaaagcctcagtgagcgagcgagcgcgc

SEQ ID NO: 112 - DNA, 51 bp, Artificial Sequence
  Description of Artificial Sequence: Synthetic oligonucleotide
gcgcgctcgctcgctcactgaggctttgcctcagtgagcgagcgagcgcgc

SEQ ID NO: 113 - DNA, 80 bp, Artificial Sequence; identical to SEQ ID NO: 65
  Description of Artificial Sequence: Synthetic oligonucleotide

SEQ ID NO: 114 - DNA, 80 bp, Artificial Sequence; identical to SEQ ID NO: 17
  Description of Artificial Sequence: Synthetic oligonucleotide

SEQ ID NO: 115 - DNA, 79 bp, Artificial Sequence
  Description of Artificial Sequence: Synthetic oligonucleotide
gcgcgctcgctcgctcactgaggcgcccgggcgtcgggcgacctttggtcgcccggcctcagtgagcgagcgagcgcgc

SEQ ID NO: 116 - DNA, 79 bp, Artificial Sequence
  Description of Artificial Sequence: Synthetic oligonucleotide
gcgcgctcgctcgctcactgaggccgggcgaccaaaggtcgcccgacgcccgggcgcctcagtgagcgagcgagcgcgc

SEQ ID NO: 117 - DNA, 89 bp, Artificial Sequence
  Description of Artificial Sequence: Synthetic oligonucleotide
gcgcgctcgctcgctcactgaggccgcccgggcaaagcccgggcgtcgggcgactttgtcgcccggcctcagtgagcgagcgagcgcgc

SEQ ID NO: 118 - DNA, 89 bp, Artificial Sequence
  Description of Artificial Sequence: Synthetic oligonucleotide
gcgcgctcgctcgctcactgaggccgggcgacaaagtcgcccgacgcccgggctttgcccgggcggcctcagtgagcgagcgagcgcgc

SEQ ID NO: 119 - DNA, 87 bp, Artificial Sequence
  Description of Artificial Sequence: Synthetic oligonucleotide
gcgcgctcgctcgctcactgaggccgcccgggcaaagcccgggcgtcgggcgattttcgcccggcctcagtgagcgagcgagcgcgc

SEQ ID NO: 120 - DNA, 87 bp, Artificial Sequence
  Description of Artificial Sequence: Synthetic oligonucleotide
gcgcgctcgctcgctcactgaggccgggcgaaaatcgcccgacgcccgggctttgcccgggcggcctcagtgagcgagcgagcgcgc

SEQ ID NO: 121 - DNA, 85 bp, Artificial Sequence
  Description of Artificial Sequence: Synthetic oligonucleotide
gcgcgctcgctcgctcactgaggccgcccgggcaaagcccgggcgtcgggcgtttcgcccggcctcagtgagcgagcgagcgcgc

SEQ ID NO: 122 - DNA, 85 bp, Artificial Sequence
  Description of Artificial Sequence: Synthetic oligonucleotide
gcgcgctcgctcgctcactgaggccgggcgaaacgcccgacgcccgggctttgcccgggcggcctcagtgagcgagcgagcgcgc

SEQ ID NO: 123 - DNA, 89 bp, Artificial Sequence
  Description of Artificial Sequence: Synthetic oligonucleotide
gcgcgctcgctcgctcactgaggccgcccgggaaacccgggcgtcgggcgacctttggtcgcccggcctcagtgagcgagcgagcgcgc

SEQ ID NO: 124 - DNA, 89 bp, Artificial Sequence
  Description of Artificial Sequence: Synthetic oligonucleotide
gcgcgctcgctcgctcactgaggccgggcgaccaaaggtcgcccgacgcccgggtttcccgggcggcctcagtgagcgagcgagcgcgc

SEQ ID NO: 125 - DNA, 87 bp, Artificial Sequence
  Description of Artificial Sequence: Synthetic oligonucleotide
gcgcgctcgctcgctcactgaggccgcccggaaaccgggcgtcgggcgacctttggtcgcccggcctcagtgagcgagcgagcgcgc

SEQ ID NO: 126 - DNA, 87 bp, Artificial Sequence
  Description of Artificial Sequence: Synthetic oligonucleotide
gcgcgctcgctcgctcactgaggccgggcgaccaaaggtcgcccgacgcccggtttccgggcggcctcagtgagcgagcgagcgcgc

SEQ ID NO: 127 - DNA, 85 bp, Artificial Sequence
  Description of Artificial Sequence: Synthetic oligonucleotide
gcgcgctcgctcgctcactgaggccgcccgaaacgggcgtcgggcgacctttggtcgcccggcctcagtgagcgagcgagcgcgc

SEQ ID NO: 128 - DNA, 85 bp, Artificial Sequence
  Description of Artificial Sequence: Synthetic oligonucleotide
gcgcgctcgctcgctcactgaggccgggcgaccaaaggtcgcccgacgcccgtttcgggcggcctcagtgagcgagcgagcgcgc

SEQ ID NO: 129 - DNA, 83 bp, Artificial Sequence
  Description of Artificial Sequence: Synthetic oligonucleotide
gcgcgctcgctcgctcactgaggccgcccaaagggcgtcgggcgacctttggtcgcccggcctcagtgagcgagcgagcgcgc

SEQ ID NO: 130 - DNA, 83 bp, Artificial Sequence
  Description of Artificial Sequence: Synthetic oligonucleotide
gcgcgctcgctcgctcactgaggccgggcgaccaaaggtcgcccgacgccctttgggcggcctcagtgagcgagcgagcgcgc

SEQ ID NO: 131 - DNA, 81 bp, Artificial Sequence
  Description of Artificial Sequence: Synthetic oligonucleotide
gcgcgctcgctcgctcactgaggccgccaaaggcgtcgggcgacctttggtcgcccggcctcagtgagcgagcgagcgcgc

SEQ ID NO: 132 - DNA, 81 bp, Artificial Sequence
  Description of Artificial Sequence: Synthetic oligonucleotide
gcgcgctcgctcgctcactgaggccgggcgaccaaaggtcgcccgacgcctttggcggcctcagtgagcgagcgagcgcgc

SEQ ID NO: 133 - DNA, 79 bp, Artificial Sequence
  Description of Artificial Sequence: Synthetic oligonucleotide
gcgcgctcgctcgctcactgaggccgcaaagcgtcgggcgacctttggtcgcccggcctcagtgagcgagcgagcgcgc

SEQ ID NO: 134 - DNA, 79 bp, Artificial Sequence
  Description of Artificial Sequence: Synthetic oligonucleotide
gcgcgctcgctcgctcactgaggccgggcgaccaaaggtcgcccgacgctttgcggcctcagtgagcgagcgagcgcgc

SEQ ID NO: 135 - DNA, 546 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatccggactctaaggtaaatataaaatttttaagtgtataatgtgttaaactactgattctaattgtttctctcttttagattccaacctttggaactg

SEQ ID NO: 136-150 - skipped (15 entries)

SEQ ID NO: 151 - DNA, 1530 bp, Homo sapiens
atgtccatgggggcaccgcggtccctcctcctggccctggctgctggcctggccgttgcccgtccgcccaacatcgtgctgatctttgccgacgacctcggctatggggacctgggctgctatgggcaccccagctctaccactcccaacctggaccagctggcggcgggagggctgcggttcacagacttctacgtgcctgtgtctctgtgcacaccctctagggccgccctcctgaccggccggctcccggttcggatgggcatgtaccctggcgtcctggtgcccagctcccgggggggcctgcccctggaggaggtgaccgtggccgaagtcctggctgcccgaggctacctcacaggaatggccggcaagtggcaccttggggtggggcctgagggggccttcctgcccccccatcagggcttccatcgatttctaggcatcccgtactcccacgaccagggcccctgccagaacctgacctgcttcccgccggccactccttgcgacggtggctgtgaccagggcctggtccccatcccactgttggccaacctgtccgtggaggcgcagcccccctggctgcccggactagaggcccgctacatggctttcgcccatgacctcatggccgacgcccagcgccaggatcgccccttcttcctgtactatgcctctcaccacacccactaccctcagttcagtgggcagagctttgcagagcgttcaggccgcgggccatttggggactccctgatggagctggatgcagctgtggggaccctgatgacagccataggggacctggggctgcttgaagagacgctggtcatcttcactgcagacaatggacctgagaccatgcgtatgtcccgaggcggctgctccggtctcttgcggtgtggaaagggaacgacctacgagggcggtgtccgagagcctgccttggccttctggccaggtcatatcgctcccggcgtgacccacgagctggccagctccctggacctgctgcctaccctggcagccctggctggggccccactgcccaatgtcaccttggatggctttgacctcagccccctgctgctgggcacaggcaagagccctcggcagtctctcttcttctacccgtcctacccagacgaggtccgtggggtttttgctgtgcggactggaaagtacaaggctcacttcttcacccagggctctgcccacagtgataccactgcagaccctgcctgccacgcctccagctctctgactgctcatgagcccccgctgctctatgacctgtccaaggaccctggtgagaactacaacctgctggggggtgtggccggggccaccccagaggtgctgcaagccctgaaacagcttcagctgctcaaggcccagttagacgcagctgtgaccttcggccccagccaggtggcccggggcgaggaccccgccctgcagatctgctgtcatcctggctgcaccccccgcccagcttgctgccattgcccagatccccatgcctga

SEQ ID NO: 152 - DNA, 1653 bp, Homo sapiens
atgccgccaccccggaccggccgaggccttctctggctgggtctggttctgagctccgtctgcgtcgccctcggatccgaaacgcaggccaactcgaccacagatgctctgaacgttcttctcatcatcgtggatgacctgcgcccctccctgggctgttatggggataagctggtgaggtccccaaatattgaccaactggcatcccacagcctcctcttccagaatgcctttgcgcagcaagcagtgtgcgccccgagccgcgtttctttcctcactggcaggagacctgacaccacccgcctgtacgacttcaactcctactggagggtgcacgctggaaacttctccaccatcccccagtacttcaaggagaatggctatgtgaccatgtcggtgggaaaagtctttcaccctgggatatcttctaaccataccgatgattctccgtatagctggtcttttccaccttatcatccttcctctgagaagtatgaaaacactaagacatgtcgagggccagatggagaactccatgccaacctgctttgccctgtggatgtgctggatgttcccgagggcaccttgcctgacaaacagagcactgagcaagccatacagttgttggaaaagatgaaaacgtcagccagtcctttcttcctggccgttgggtatcataagccacacatccccttcagataccccaaggaatttcagaagttgtatcccttggagaacatcaccctggcccccgatcccgaggtccctgatggcctaccccctgtggcctacaacccctggatggacatcaggcaacgggaagacgtccaagccttaaacatcagtgtgccgtatggtccaattcctgtggactttcagcggaaaatccgccagagctactttgcctctgtgtcatatttggatacacaggtcggccgcctcttgagtgctttggacgatcttcagctggccaacagcaccatcattgcatttacctcggatcatgggtgggctctaggtgaacatggagaatgggccaaatacagcaattttgatgttgctacccatgttcccctgatattctatgttcctggaaggacggcttcacttccggaggcaggcgagaagcttttcccttacctcgacccttttgattccgcctcacagttgatggagccaggcaggcaatccatggaccttgtggaacttgtgtctctttttcccacgctggctggacttgcaggactgcaggttccacctcgctgccccgttccttcatttcacgttgagctgtgcagagaaggcaagaaccttctgaagcattttcgattccgtgacttggaagaggatccgtacctccctggtaatccccgtgaactgattgcctatagccagtatccccggccttcagacatccctcagtggaattctgacaagccgagtttaaaagatataaagatcatgggctattccatacgcaccatagactataggtatactgtgtgggttggcttcaatcctgatgaatttctagctaacttttctgacatccatgcaggggaactgtattttgtggattctgacccattgcaggatcacaatatgtataatgattcccaaggtggagatcttttccagttgttgatgccttga

SEQ ID NO: 153-300 - skipped (148 entries)

SEQ ID NO: 301 - DNA, 16 bp, Artificial Sequence
  Description of Artificial Sequence: Synthetic oligonucleotide
gcgcgctcgctcgctc

SEQ ID NO: 302 - DNA, 8 bp, Artificial Sequence
  Description of Artificial Sequence: Synthetic oligonucleotide
actgaggc

SEQ ID NO: 303 - DNA, 22 bp, Artificial Sequence
  Description of Artificial Sequence: Synthetic oligonucleotide
cgggcgaccaaaggtcgcccga

SEQ ID NO: 304 - DNA, 10 bp, Artificial Sequence
  Description of Artificial Sequence: Synthetic oligonucleotide
cgcccgggcg

SEQ ID NO: 305 - DNA, 8 bp, Artificial Sequence
  Description of Artificial Sequence: Synthetic oligonucleotide
gcctcagt

SEQ ID NO: 306 - DNA, 16 bp, Artificial Sequence
  Description of Artificial Sequence: Synthetic oligonucleotide
gagcgagcgagcgcgc

SEQ ID NO: 307 - skipped

SEQ ID NO: 308 - DNA, 317 bp, Artificial Sequence
  Description of Artificial Sequence: Synthetic polynucleotide
ggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 309 - DNA, 576 bp, Artificial Sequence
  Description of Artificial Sequence: Synthetic polynucleotide
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 310 - DNA, 1313 bp, Artificial Sequence
  Description of Artificial Sequence: Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaa

SEQ ID NO: 311-312 - skipped (2 entries)

SEQ ID NO: 313 - protein, 19 aa, Artificial Sequence
  Description of Artificial Sequence: Synthetic peptide
MDWTWRILFLVAAATGAHS

SEQ ID NO: 314 - protein, 19 aa, Artificial Sequence
  Description of Artificial Sequence: Synthetic peptide
MLPSQLIGFLLLWVPASRG

SEQ ID NO: 315 - protein, 7 aa, Simian virus 40
PKKKRKV

SEQ ID NO: 316 - protein, 16 aa, Unknown
  Description of Unknown: Nucleoplasmin bipartite NLS sequence
KRPAATKKAGQAKKKK

SEQ ID NO: 317 - protein, 9 aa, Unknown
  Description of Unknown: C-myc NLS sequence
PAAKRVKLD

SEQ ID NO: 318 - protein, 11 aa, Unknown
  Description of Unknown: C-myc NLS sequence
RQRRNELKRSP

SEQ ID NO: 319 - protein, 38 aa, Homo sapiens
NQSSNFGPMKGGNFGGRSSGPYGGGGQYFAKPRNQGGY

SEQ ID NO: 320 - protein, 42 aa, Unknown
  Description of Unknown: IBB domain from importin-alpha sequence
RMRIZFKNKGKDTAELRRRRVEVSVELRKAKKDEQILKRRNV

SEQ ID NO: 321 - protein, 8 aa, Unknown
  Description of Unknown: Myoma T protein sequence
VSRKRPRP

SEQ ID NO: 322 - protein, 8 aa, Unknown
  Description of Unknown: Myoma T protein sequence
PPKKARED

SEQ ID NO: 323 - protein, 8 aa, Homo sapiens
PQPKKKPL

SEQ ID NO: 324 - protein, 12 aa, Mus musculus
SALIKKKKKMAP

SEQ ID NO: 325 - protein, 5 aa, Influenza virus
DRLRR

SEQ ID NO: 326 - protein, 7 aa, Influenza virus
PKQKKRK

SEQ ID NO: 327 - protein, 10 aa, Hepatitis delta virus
RKLKKKIKKL

SEQ ID NO: 328 - protein, 10 aa, Mus musculus
REKKKFLKRR

SEQ ID NO: 329 - protein, 20 aa, Homo sapiens
KRKGDEVDGVDEVAKKKSKK

SEQ ID NO: 330 - protein, 17 aa, Homo sapiens
RKCLQAGMNLEARKTKK

SEQ ID NO: 331-499 - skipped (169 entries)

SEQ ID NO: 500 - DNA, 43 bp, Artificial Sequence
  Description of Artificial Sequence: Synthetic oligonucleotide
gcccgctggtttccagcgggctgcgggcccgaaacgggcccgc

SEQ ID NO: 501 - DNA, 28 bp, Artificial Sequence
  Description of Artificial Sequence: Synthetic oligonucleotide
cgggcccgtgcgggcccaaagggcccgc

SEQ ID NO: 502 - DNA, 28 bp, Artificial Sequence
  Description of Artificial Sequence: Synthetic oligonucleotide
gcccgggcacgcccgggtttcccgggcg

SEQ ID NO: 503 - DNA, 22 bp, Artificial Sequence
  Description of Artificial Sequence: Synthetic oligonucleotide
cgtgcgggcccaaagggcccgc

SEQ ID NO: 504 - DNA, 21 bp, Artificial Sequence
  Description of Artificial Sequence: Synthetic oligonucleotide
cgggcgaccaaaggtcgcccg

SEQ ID NO: 505 - DNA, 20 bp, Artificial Sequence
  Description of Artificial Sequence: Synthetic oligonucleotide
cgcccgggctttgcccgggc

SEQ ID NO: 506 - DNA, 42 bp, Artificial Sequence
  Description of Artificial Sequence: Synthetic oligonucleotide
cgggcgaccaaaggtcgcccgacgcccgggctttgcccgggc

SEQ ID NO: 507 - DNA, 21 bp, Artificial Sequence
  Description of Artificial Sequence: Synthetic oligonucleotide
cgggcgaccaaaggtcgcccg

SEQ ID NO: 508 - DNA, 20 bp, Artificial Sequence
  Description of Artificial Sequence: Synthetic oligonucleotide
cgcccgggctttgcccgggc

SEQ ID NO: 509 - DNA, 34 bp, Artificial Sequence
  Description of Artificial Sequence: Synthetic oligonucleotide
cgggcgaccaaaggtcgcccgacgcccgggcggc

SEQ ID NO: 510 - DNA, 21 bp, Artificial Sequence
  Description of Artificial Sequence: Synthetic oligonucleotide
cgggcgaccaaaggtcgcccg

SEQ ID NO: 511 - DNA, 20 bp, Artificial Sequence
  Description of Artificial Sequence: Synthetic oligonucleotide
cgcccgggctttgcccgggc

SEQ ID NO: 512 - DNA, 30 bp, Artificial Sequence
  Description of Artificial Sequence: Synthetic oligonucleotide
cggggcccgacgcccgggctttgcccgggc

SEQ ID NO: 513 - DNA, 21 bp, Artificial Sequence
  Description of Artificial Sequence: Synthetic oligonucleotide
cgggcgaccaaaggtcgcccg

SEQ ID NO: 514 - DNA, 20 bp, Artificial Sequence
  Description of Artificial Sequence: Synthetic oligonucleotide
cgcccgggctttgcccgggc

SEQ ID NO: 515 - DNA, 29 bp, Artificial Sequence
  Description of Artificial Sequence: Synthetic oligonucleotide
cgggcccgacgcccgggctttgcccgggc

SEQ ID NO: 516 - DNA, 21 bp, Artificial Sequence
  Description of Artificial Sequence: Synthetic oligonucleotide
cgggcgaccaaaggtcgcccg

SEQ ID NO: 517 - DNA, 20 bp, Artificial Sequence
  Description of Artificial Sequence: Synthetic oligonucleotide
cgcccgggctttgcccgggc

SEQ ID NO: 518 - DNA, 20 bp, Artificial Sequence
  Description of Artificial Sequence: Synthetic oligonucleotide
gcccgggcaaagcccgggcg

SEQ ID NO: 519 - DNA, 21 bp, Artificial Sequence
  Description of Artificial Sequence: Synthetic oligonucleotide
cgggcgacctttggtcgcccg

SEQ ID NO: 520 - DNA, 42 bp, Artificial Sequence
  Description of Artificial Sequence: Synthetic oligonucleotide
gcccgggcaaagcccgggcgtcgggcgacctttggtcgcccg

SEQ ID NO: 521 - DNA, 20 bp, Artificial Sequence
  Description of Artificial Sequence: Synthetic oligonucleotide
gcccgggcaaagcccgggcg

SEQ ID NO: 522 - DNA, 21 bp, Artificial Sequence
  Description of Artificial Sequence: Synthetic oligonucleotide
cgggcgacctttggtcgcccg

SEQ ID NO: 523 - DNA, 20 bp, Artificial Sequence
  Description of Artificial Sequence: Synthetic oligonucleotide
gcccgggcaaagcccgggcg

SEQ ID NO: 524 - DNA, 21 bp, Artificial Sequence
  Description of Artificial Sequence: Synthetic oligonucleotide
cgggcgacctttggtcgcccg

SEQ ID NO: 525 - DNA, 34 bp, Artificial Sequence
  Description of Artificial Sequence: Synthetic oligonucleotide
gccgcccgggcgacgggcgacctttggtcgcccg

SEQ ID NO: 526 - DNA, 20 bp, Artificial Sequence
  Description of Artificial Sequence: Synthetic oligonucleotide
gcccgggcaaagcccgggcg

SEQ ID NO: 527 - DNA, 21 bp, Artificial Sequence
  Description of Artificial Sequence: Synthetic oligonucleotide
cgggcgacctttggtcgcccg

SEQ ID NO: 528 - DNA, 20 bp, Artificial Sequence
  Description of Artificial Sequence: Synthetic oligonucleotide
gcccgggcaaagcccgggcg

SEQ ID NO: 529 - DNA, 21 bp, Artificial Sequence
  Description of Artificial Sequence: Synthetic oligonucleotide
cgggcgacctttggtcgcccg

SEQ ID NO: 530 - DNA, 4470 bp, Homo sapiens
gtagatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgggaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttttctcttctctgtctaactttctttttcctatcttttttcttctttgttctgtttacttttgctcatatcatcattcccgctatctttctattaactgaccataacacagaactagttgactatatattatgttgaaattttatggcagctatttatttatttaaattttttgtaacagttttgttttctaataagaaaaatccatgctttttgtagctggttgaaaattcaggaatatgtaaaactttttggtatttaattaaattgattccttttcttaattttaaaaaaaaaaaaaaa

SEQ ID NO: 531 - DNA, 3845 bp, Homo sapiens
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 532 - DNA, 3545 bp, Homo sapiens
gttcctgaggctggcatctggggaaacctttcttccatggctcaggacacactcctggatcgagccaacaggagaactttctgtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 533 - DNA, 4015 bp, Homo sapiens
gttcctgaggctggcatctggatgaggaaactgaagttgaggaatagtgaagagtttgtccaatgtcatagccccgtaatcaacgggacaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatggattggtgaacagccacctcacttccagtttttgccggggcctcttttcagttctgagcaccagccagagtctaactgaattggacctcagtgacaattctctgggggacccagggatgagagtgttgtgtgaaacgctccagcatcctggctgtaacattcggagattgtggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 534 - DNA, 4016 bp, Homo sapiens
gttcctgaggctggcatctggatgaggaaactgaagttgaggaatagtgaagagtttgtccaatgtcatagccccgtaatcaacgggacaaaaattttcttgctgatgggtcaagatggcatcgtgaagtggttgttcaccgtaaactgtaatacaatcctgtttatggatttgtttgcatatttttccctccatagggaaacctttcttccatggctcaggacacactcctggatcgagccaacaggagaactttctggtaagcatttggctaacttttttttttttgagatggagtcttgctgtgtcgcctaggctggagtgcagtggcgtgatcttggctcactgcagcctccacttcccgggttcaatcaattctcctacctcaacttcctgagtagctgggattacaggcgcccgccaccacacccggctcatttttgtacttttagtagagacacagttttgccatgttggccaggctggtcttgaattcctcagctcaggtgatctgcctgccttggcctctcaaagtgctgggattacaggcgtgagccactgtgcccggccttggctaacttttcaaaattaaagattttgacttgttacagtcatgtgacatttttttctttctgtttgctgagtttttgataatttatatctctcaaagtggagactttaaaaaagactcatccgtgtgccgtgttcactgcctggtatcttagtgtggaccgaagcctaaggaccctgaaaacagctgcagatgaagatggcaagcacccgctgcaagctggccaggtacctggaggacctggaggatgtggacttgaagaaatttaagatgcacttagaggactatcctccccagaagggctgcatccccctcccgaggggtcagacagagaaggcagaccatgtggatctagccacgctaatgatcgacttcaatggggaggagaaggcgtgggccatggccgtgtggatcttcgctgcgatcaacaggagagacctttatgagaaagcaaaaagagatgagccgaagtggggttcagataatgcacgtgtttcgaatcccactgtgatatgccaggaagacagcattgaagaggagtggatgggtttactggagtacctttcgagaatctctatttgtaaaatgaagaaagattaccgtaagaagtacagaaagtacgtgagaagcagattccagtgcattgaagacaggaatgcccgtctgggtgagagtgtgagcctcaacaaacgctacacacgactgcgtctcatcaaggagcaccggagccagcaggagagggagcaggagcttctggccatcggcaagaccaagacgtgtgagagccccgtgagtcccattaagatggagttgctgtttgaccccgatgatgagcattctgagcctgtgcacaccgtggtgttccagggggcggcagggattgggaaaacaatcctggccaggaagatgatgttggactgggcgtcggggacactctaccaagacaggtttgactatctgttctatatccactgtcgagaggtgagccttgtgacacagaggagcctgggggacctgatcatgagctgctgccccgacccaaacccacccatccacaagatcgtgagaaaaccctccagaatcctcttcctcatggacggcttcgatgagctgcaaggtgcctttgacgagcacataggaccgctctgcactgactggcagaaggccgagcggggagacattctcctgagcagcctcatcagaaagaagctgcttcccgaggcctctctgctcatcaccacgagacctgtggccctggagaaactgcagcacttgctggaccatcctcggcatgtggagatcctgggtttctccgaggccaaaaggaaagagtacttcttcaagtacttctctgatgaggcccaagccagggcagccttcagtctgattcaggagaacgaggtcctcttcaccatgtgcttcatccccctggtctgctggatcgtgtgcactggactgaaacagcagatggagagtggcaagagccttgcccagacatccaagaccaccaccgcggtgtacgtcttcttcctttccagtttgctgcagccccggggagggagccaggagcacggcctctgcgcccacctctgggggctctgctctttggctgcagatggaatctggaaccagaaaatcctgtttgaggagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcaagatctctcagcaaatcaggctggagctgctgaaatggattgaagtgaaagccaaagctaaaaagctgcagatccagcccagccagctggaattgttctactgtttgtacgagatgcaggaggaggacttcgtgcaaagggccatggactatttccccaagattgagatcaatctctccaccagaatggaccacatggtttcttccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcgcatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtcagctgctgcctcacatcagcatgttgtcaggatcttgcatcagtattgagcaccagccattccctgaccagactctatgtgggggagaatgccttgggagactcaggagtcgcaattttatgtgaaaaagccaagaatccacagtgtaacctgcagaaactggggttggtgaattctggccttacgtcagtctgttgttcagctttgtcctcggtactcagcactaatcagaatctcacgcacctttacctgcgaggcaacactctcggagacaaggggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctcttggtgacctcatgtaattagctcattcaataaagcactttctttatttt

SEQ ID NO: 535-537 - skipped (3 entries)

SEQ ID NO: 538 - protein, 14 aa, Artificial Sequence
  Description of Artificial Sequence: Synthetic peptide
NEAYVHDAPVRSLN

SEQ ID NO: 539 - protein, 1036 aa, Homo sapiens
MKMASTRCKLARYLEDLEDVDLKKFKMHLEDYPPQKGCIPLPRGQTEKADHVDLATLMIDFNGEEKAWAMAVWIFAAINRRDLYEKAKRDEPKWGSDNARVSNPTVICQEDSIEEEWMGLLEYLSRISICKMKKDYRKKYRKYVRSRFQCIEDRNARLGESVSLNKRYTRLRLIKEHRSQQEREQELLAIGKTKTCESPVSPIKMELLFDPDDEHSEPVHTVVFQGAAGIGKTILARKMMLDWASGTLYQDRFDYLFYIHCREVSLVTQRSLGDLIMSCCPDPNPPIHKIVRKPSRILFLMDGFDELQGAFDEHIGPLCTDWQKAERGDILLSSLIRKKLLPEASLLITTRPVALEKLQHLLDHPRHVEILGFSEAKRKEYFFKYFSDEAQARAAFSLIQENEVLFTMCFIPLVCWIVCTGLKQQMESGKSLAQTSKTTTAVYVFFLSSLLQPRGGSQEHGLCAHLWGLCSLAADGIWNQKILFEESDLRNHGLQKADVSAFLRMNLFQKEVDCEKFYSFIHMTFQEFFAAMYYLLEEEKEGRTNVPGSRLKLPSRDVTVLLENYGKFEKGYLIFVVRFLFGLVNQERTSYLEKKLSCKISQQIRLELLKWIEVKAKAKKLQIQPSQLELFYCLYEMQEEDFVQRAMDYFPKIEINLSTRMDHMVSSFCIENCHRVESLSLGFLHNMPKEEEEEEKEGRHLDMVQCVLPSSSHAACSHGLVNSHLTSSFCRGLFSVLSTSQSLTELDLSDNSLGDPGMRVLCETLQHPGCNIRRLWLGRCGLSHECCFDISLVLSSNQKLVELDLSDNALGDFGIRLLCVGLKHLLCNLKKLWLVSCCLTSACCQDLASVLSTSHSLTRLYVGENALGDSGVAILCEKAKNPQCNLQKLGLVNSGLTSVCCSALSSVLSTNQNLTHLYLRGNTLGDKGIKLLCEGLLHPDCKLQVLELDNCNLTSHCCWDLSTLLTSSQSLRKLSLGNNDLGDLGVMMFCEVLKQQSCLLQNLGLSEMYFNYETKSALETLQEEKPELTVVFEPSW

SEQ ID NO: 540 - DNA, 21 bp, Artificial Sequence
  Description of Artificial Sequence: Synthetic oligonucleotide
  Description of Combined DNA/RNA Molecule: Synthetic oligonucleotide
gugcauugaagacaggaautt

SEQ ID NO: 541 - DNA, 21 bp, Homo sapiens
ggctgtaacattcggagattg

SEQ ID NO: 542 - DNA, 21 bp, Homo sapiens
tcatcattcccgctatctttc

SEQ ID NO: 543 - DNA, 21 bp, Homo sapiens
ccgtaagaagtacagaaagta

SEQ ID NO: 544 - DNA, 21 bp, Homo sapiens
gagactcaggagtcgcaattt

SEQ ID NO: 545 - DNA, 21 bp, Homo sapiens
cctcatgtaattagctcattc

SEQ ID NO: 546 - DNA, 21 bp, Homo sapiens
gtggatctagccacgctaatg

SEQ ID NO: 547 - DNA, 21 bp, Homo sapiens
ccacagtgtaacctgcagaaa

SEQ ID NO: 548 - DNA, 21 bp, Homo sapiens
ccagccagagtctaactgaat

SEQ ID NO: 549 - DNA, 21 bp, Homo sapiens
gcgttagaaacacttcaagaa

SEQ ID NO: 550 - DNA, 21 bp, Homo sapiens
gctggaattgttctactgttt

SEQ ID NO: 551 - DNA, 21 bp, Homo sapiens
ccacatgactttccaggagtt

SEQ ID NO: 552 - DNA, 21 bp, Homo sapiens
ggctgtaacattcggagattg

SEQ ID NO: 553 - DNA, 58 bp, Artificial Sequence
  Description of Artificial Sequence: Synthetic oligonucleotide
ccggggctgtaacattcggagattgctcgagcaatctccgaatgttacagcctttttg

SEQ ID NO: 554 - DNA, 58 bp, Artificial Sequence
  Description of Artificial Sequence: Synthetic oligonucleotide
aattcaaaaaggctgtaacattcggagattgctcgagcaatctccgaatgttacagcc

SEQ ID NO: 555 - DNA, 21 bp, Homo sapiens
tcatcattcccgctatctttc

SEQ ID NO: 556 - DNA, 58 bp, Artificial Sequence
  Description of Artificial Sequence: Synthetic oligonucleotide
ccggtcatcattcccgctatctttcctcgaggaaagatagcgggaatgatgatttttg

SEQ ID NO: 557 - DNA, 58 bp, Artificial Sequence
  Description of Artificial Sequence: Synthetic oligonucleotide
aattcaaaaatcatcattcccgctatctttcctcgaggaaagatagcgggaatgatga

SEQ ID NO: 558 - DNA, 21 bp, Homo sapiens
ccgtaagaagtacagaaagta

SEQ ID NO: 559 - DNA, 58 bp, Artificial Sequence
  Description of Artificial Sequence: Synthetic oligonucleotide
ccggccgtaagaagtacagaaagtactcgagtactttctgtacttcttacggtttttg

SEQ ID NO: 560 - DNA, 58 bp, Artificial Sequence
  Description of Artificial Sequence: Synthetic oligonucleotide
aattcaaaaaccgtaagaagtacagaaagtactcgagtactttctgtacttcttacgg

SEQ ID NO: 561 - DNA, 21 bp, Homo sapiens
gagactcaggagtcgcaattt

SEQ ID NO: 562 - DNA, 58 bp, Artificial Sequence
  Description of Artificial Sequence: Synthetic oligonucleotide
ccgggagactcaggagtcgcaatttctcgagaaattgcgactcctgagtctctttttg

SEQ ID NO: 563 - DNA, 58 bp, Artificial Sequence
  Description of Artificial Sequence: Synthetic oligonucleotide
aattcaaaaagagactcaggagtcgcaatttctcgagaaattgcgactcctgagtctc

SEQ ID NO: 564 - DNA, 21 bp, Homo sapiens
cctcatgtaattagctcattc

SEQ ID NO: 565 - DNA, 58 bp, Artificial Sequence
  Description of Artificial Sequence: Synthetic oligonucleotide
ccggcctcatgtaattagctcattcctcgaggaatgagctaattacatgaggtttttg

SEQ ID NO: 566 - DNA, 58 bp, Artificial Sequence
  Description of Artificial Sequence: Synthetic oligonucleotide
aattcaaaaacctcatgtaattagctcattcctcgaggaatgagctaattacatgagg

SEQ ID NO: 567 - DNA, 21 bp, Homo sapiens
gtggatctagccacgctaatg

SEQ ID NO: 568 - DNA, 58 bp, Artificial Sequence
  Description of Artificial Sequence: Synthetic oligonucleotide
ccgggtggatctagccacgctaatgctcgagcattagcgtggctagatccactttttg

SEQ ID NO: 569 - DNA, 58 bp, Artificial Sequence
  Description of Artificial Sequence: Synthetic oligonucleotide
aattcaaaaagtggatctagccacgctaatgctcgagcattagcgtggctagatccac

SEQ ID NO: 570 - DNA, 21 bp, Homo sapiens
ccacagtgtaacctgcagaaa

SEQ ID NO: 571 - DNA, 58 bp, Artificial Sequence
  Description of Artificial Sequence: Synthetic oligonucleotide
aattcaaaaaccacagtgtaacctgcagaaactcgagtttctgcaggttacactgtgg

SEQ ID NO: 572 - DNA, 21 bp, Homo sapiens
ccagccagagtctaactgaat

SEQ ID NO: 573 - DNA, 58 bp, Artificial Sequence
  Description of Artificial Sequence: Synthetic oligonucleotide
ccggccagccagagtctaactgaatctcgagattcagttagactctggctggtttttg

SEQ ID NO: 574 - DNA, 58 bp, Artificial Sequence
  Description of Artificial Sequence: Synthetic oligonucleotide
aattcaaaaaccagccagagtctaactgaatctcgagattcagttagactctggctgg

SEQ ID NO: 575 - DNA, 21 bp, Homo sapiens
gcgttagaaacacttcaagaa

SEQ ID NO: 576 - DNA, 58 bp, Artificial Sequence
  Description of Artificial Sequence: Synthetic oligonucleotide
ccgggcgttagaaacacttcaagaactcgagttcttgaagtgtttctaacgctttttg

SEQ ID NO: 577 - DNA, 58 bp, Artificial Sequence
  Description of Artificial Sequence: Synthetic oligonucleotide
aattcaaaaagcgttagaaacacttcaagaactcgagttcttgaagtgtttctaacgc

SEQ ID NO: 578 - DNA, 21 bp, Homo sapiens
gctggaattgttctactgttt

SEQ ID NO: 579 - DNA, 58 bp, Artificial Sequence
  Description of Artificial Sequence: Synthetic oligonucleotide
ccgggctggaattgttctactgtttctcgagaaacagtagaacaattccagctttttg

SEQ ID NO: 580 - DNA, 58 bp, Artificial Sequence
  Description of Artificial Sequence: Synthetic oligonucleotide
aattcaaaaagctggaattgttctactgtttctcgagaaacagtagaacaattccagc

SEQ ID NO: 581 - DNA, 21 bp, Homo sapiens
ccacatgactttccaggagtt

SEQ ID NO: 582 - DNA, 58 bp, Artificial Sequence
  Description of Artificial Sequence: Synthetic oligonucleotide
ccggccacatgactttccaggagttctcgagaactcctggaaagtcatgtggtttttg

SEQ ID NO: 583 - DNA, 58 bp, Artificial Sequence
  Description of Artificial Sequence: Synthetic oligonucleotide
aattcaaaaaccacatgactttccaggagttctcgagaactcctggaaagtcatgtgg

SEQ ID NO: 584 - DNA, 21 bp, Homo sapiens
gaaagccaaagctaagaagta

SEQ ID NO: 585 - DNA, 58 bp, Artificial Sequence
  Description of Artificial Sequence: Synthetic oligonucleotide
ccgggaaagccaaagctaagaagtactcgagtacttcttagctttggctttctttttg

SEQ ID NO: 586 - DNA, 58 bp, Artificial Sequence
  Description of Artificial Sequence: Synthetic oligonucleotide
aattcaaaaagaaagccaaagctaagaagtactcgagtacttcttagctttggctttc

SEQ ID NO: 587 - RNA, 23 nt, Homo sapiens
ucuuugguuaucuagcuguauga

SEQ ID NO: 588 - RNA, 23 nt, Homo sapiens
ucuuugguuaucuagcuguauga

SEQ ID NO: 589 - DNA, 22 bp, Artificial Sequence
  Description of Artificial Sequence: Synthetic oligonucleotide
tggggtatttgacaaactgaca

SEQ ID NO: 590 - RNA, 63 nt, Caenorhabditis brenneri
ucgcccaucccguuguuccaauauuccaacaacaagugauuauugagcaaugcgcaugugcgg

SEQ ID NO: 591 - RNA, 97 nt, Caenorhabditis briggsae
aagcauuuuucugucccgcgcaucccuuuguuccaauauucaaaccaguagaaagauuauugagcaaugcgcaugugcgggacagauugaauagcug

SEQ ID NO: 592 - RNA, 98 nt, Caenorhabditis elegans
auauagcaucuuucugucucgcccaucccguugcuccaauauucuaacaacaagugauuauugagcaaugcgcaugugcgggauagacugauggcugc

SEQ ID NO: 593 - RNA, 101 nt, Caenorhabditis remanei
ugaagcgucucucugucccgcucauccuguuguuccaauauuccaacagcccagugauuauugagcaaugcgcaugugcgggacagauuguaugcugccau

SEQ ID NO: 594 - RNA, 22 nt, Homo sapiens
aguucuucaguggcaagcuuua

SEQ ID NO: 595 - RNA, 85 nt, Homo sapiens
ggcugagccgcaguaguucuucaguggcaagcuuuauguccugacccagcuaaagcugccaguugaagaacuguugcccucugcc

SEQ ID NO: 596 - RNA, 21 nt, Mus musculus
gugcauuguaguugcauugca

SEQ ID NO: 597 - RNA, 69 nt, Mus musculus
cuguggugcauuguaguugcauugcauguucuggcaauaccugugcaauguuuccacagugcaucacgg

SEQ ID NO: 598 - protein, 343 aa, Homo sapiens
MESKYKEILLLTGLDNITDEELDRFKFFLSDEFNIATGKLHTANRIQVATLMIQNAGAVSAVMKTIRIFQKLNYMLLAKRLQEEKEKVDKQYKSVTKPKPLSQAEMSPAASAAIRNDVAKQRAAPKVSPHVKPEQKQMVAQQESIREGFQKRCLPVMVLKAKKPFTFETQEGKQEMFHATVATEKEFFFVKVFNTLLKDKFIPKRIIIIARYYRHSGFLEVNSASRVLDAESDQKVNVPLNIIRKAGETPKINTLQTQPLGTIVNGLFVVQKVTEKKKNILFDLSDNTGKMEVLGVRNEDTMKCKEGDKVRLTFFTLSKNGEKLQLTSGVHSTIKVIKAKKKT

SEQ ID NO: 599 - skipped

SEQ ID NO: 600 - DNA, 1558 bp, Homo sapiens
atagacattttcttctgtggctgctagtgagaacccaaaccagctcagccaattagagctccagttgtcactcctacccacactgggcctgggggtgaagggaagtgtttattaggggtacatgtgaagccgtccagaagtgtcagagtctttgtagctttgaaagtcacctaggttatttgggcatgctctcctgagtcctctgctagttaagctctctgaaaagaaggtggcagacccggtttgctgatcgccccagggatcaggaggctgatcccaaagttgtcagatggagagtaaatacaaggagatactcttgctaacaggcctggataacatcactgatgaggaactggataggtttaagttctttctttcagacgagtttaatattgccacaggcaaactacatactgcaaacagaatacaagtagctaccttgatgattcaaaatgctggggcggtgtctgcagtgatgaagaccattcgtatttttcagaagttgaattatatgcttttggcaaaacgtcttcaggaggagaaggagaaagttgataagcaatacaaatcggtaacaaaaccaaagccactaagtcaagctgaaatgagtcctgctgcatctgcagccatcagaaatgatgtcgcaaagcaacgtgctgcaccaaaagtctctcctcatgttaagcctgaacagaaacagatggtggcccagcaggaatctatcagagaagggtttcagaagcgctgtttgccagttatggtactgaaagcaaagaagcccttcacgtttgagacccaagaaggcaagcaggagatgtttcatgctacagtggctacagaaaaggaattcttctttgtaaaagtttttaatacactgctgaaagataaattcattccaaagagaataattataatagcaagatattatcggcacagtggtttcttagaggtaaatagcgcctcacgtgtgttagatgctgaatctgaccaaaaggttaatgtcccgctgaacattatcagaaaagctggtgaaaccccgaagatcaacacgcttcaaactcagccccttggaacaattgtgaatggtttgtttgtagtccagaaggtaacagaaaagaagaaaaacatattatttgacctaagtgacaacactgggaaaatggaagtactgggggttagaaacgaggacacaatgaaatgtaaggaaggagataaggttcgacttacattcttcacactgtcaaaaaatggagaaaaactacagctgacatctggagttcatagcaccataaaggttattaaggccaaaaaaaaaacatagagaagtaaaaaggaccaattcaagccaactggtctaagcagcatttaattgaagaatatgtgatacagcctcttcaatcagattgtaagttacctgaaagctgcagttcacaggctcctctctccaccaaattaggatagaataattgctggataaacaaattcagaatatcaacagatgatcacaataaacatctgtttctcattcaaaaaaaaaaaaaaaaaaaaaaaaaaaaaa

SEQ ID NO: 601 - DNA, 21 bp, Artificial Sequence
  Description of Artificial Sequence: Synthetic oligonucleotide
cccgaagatcaacacgcttca

SEQ ID NO: 602 - DNA, 24 bp, Artificial Sequence
  Description of Artificial Sequence: Synthetic oligonucleotide
ttagggttagggttagggttaggg

SEQ ID NO: 603 - DNA, 24 bp, Artificial Sequence
  Description of Artificial Sequence: Synthetic oligonucleotide
ttcaaattcaaattcaaattcaaa

SEQ ID NO: 604 - DNA, 6 bp, Artificial Sequence
  Description of Artificial Sequence: Synthetic oligonucleotide
ttaggg

SEQ ID NO: 605 - DNA, 21 bp, Homo sapiens
agccactaagtcaagctgaaa

SEQ ID NO: 606 - DNA, 21 bp, Homo sapiens
ccaactggtctaagcagcatt

SEQ ID NO: 607 - DNA, 21 bp, Homo sapiens
gaaacgaggacacaatgaaat

SEQ ID NO: 608 - DNA, 21 bp, Homo sapiens
gccactaagtcaagctgaaat

SEQ ID NO: 609 - DNA, 21 bp, Homo sapiens
ctggagttcatagcaccataa

SEQ ID NO: 610 - DNA, 21 bp, Homo sapiens
cccgctgaacattatcagaaa

SEQ ID NO: 611 - DNA, 1393 bp, Homo sapiens
atactttcagtttcagtcacacaagaagggaggagagaaaagccatggccgacaaggtcctgaaggagaagagaaagctgtttatccgttccatgggtgaaggtacaataaatggcttactggatgaattattacagacaagggtgctgaacaaggaagagatggagaaagtaaaacgtgaaaatgctacagttatggataagacccgagctttgattgactccgttattccgaaaggggcacaggcatgccaaatttgcatcacatacatttgtgaagaagacagttacctggcagggacgctgggactctcagcagatcaaacatctggaaattaccttaatatgcaagactctcaaggagtactttcttcctttccagctcctcaggcagtgcaggacaacccagctatgcccacatcctcaggctcagaagggaatgtcaagctttgctccctagaagaagctcaaaggatatggaaacaaaagtcggcagagatttatccaataatggacaagtcaagccgcacacgtcttgctctcattatctgcaatgaagaatttgacagtattcctagaagaactggagctgaggttgacatcacaggcatgacaatgctgctacaaaatctggggtacagcgtagatgtgaaaaaaaatctcactgcttcggacatgactacagagctggaggcatttgcacaccgcccagagcacaagacctctgacagcacgttcctggtgttcatgtctcatggtattcgggaaggcatttgtgggaagaaacactctgagcaagtcccagatatactacaactcaatgcaatctttaacatgttgaataccaagaactgcccaagtttgaaggacaaaccgaaggtgatcatcatccaggcctgccgtggtgacagccctggtgtggtgtggtttaaagattcagtaggagtttctggaaacctatctttaccaactacagaagagtttgaggatgatgctattaagaaagcccacatagagaaggattttatcgctttctgctcttccacaccagataatgtttcttggagacatcccacaatgggctctgtttttattggaagactcattgaacatatgcaagaatatgcctgttcctgtgatgtggaggaaattttccgcaaggttcgattttcatttgagcagccagatggtagagcgcagatgcccaccactgaaagagtgactttgacaagatgtttctacctcttcccaggacattaaaataaggaaactgtatgaatgtctgtgggcaggaagtgaagagatccttctgtaaaggtttttggaattatgtctgctgaataataaacttttttgaaataataaatctggtagaaaaatgaaaaaaaaaaaaa

SEQ ID NO: 612 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 613 - DNA, 21 bp, Homo sapiens
cacacgtcttgctctcattat

SEQ ID NO: 614 - DNA, 21 bp, Homo sapiens
ctacaactcaatgcaatcttt

SEQ ID NO: 615 - DNA, 21 bp, Homo sapiens
ccagatatactacaactcaat

SEQ ID NO: 616 - DNA, 21 bp, Homo sapiens
gaagagtttgaggatgatgct

SEQ ID NO: 617 - DNA, 21 bp, Homo sapiens
ccatgggtgaaggtacaataa

SEQ ID NO: 618 - DNA, 21 bp, Homo sapiens
gctttgattgactccgttatt

SEQ ID NO: 619 - DNA, 21 bp, Homo sapiens
gaaggtacaataaatggctta

SEQ ID NO: 620 - DNA, 21 bp, Homo sapiens
cacacgtcttgctctcattat

SEQ ID NO: 621 - DNA, 58 bp, Artificial Sequence
  Description of Artificial Sequence: Synthetic oligonucleotide
ccggcacacgtcttgctctcattatctcgagataatgagagcaagacgtgtgtttttg

SEQ ID NO: 622 - DNA, 58 bp, Artificial Sequence
  Description of Artificial Sequence: Synthetic oligonucleotide
aattcaaaaacacacgtcttgctctcattatctcgagataatgagagcaagacgtgtg

SEQ ID NO: 623 - DNA, 21 bp, Homo sapiens
ctacaactcaatgcaatcttt

SEQ ID NO: 624 - DNA, 58 bp, Artificial Sequence
  Description of Artificial Sequence: Synthetic oligonucleotide
ccggctacaactcaatgcaatctttctcgagaaagattgcattgagttgtagtttttg

SEQ ID NO: 625 - DNA, 58 bp, Artificial Sequence
  Description of Artificial Sequence: Synthetic oligonucleotide
aattcaaaaactacaactcaatgcaatctttctcgagaaagattgcattgagttgtag

SEQ ID NO: 626 - DNA, 21 bp, Homo sapiens
ccagatatactacaactcaat

SEQ ID NO: 627 - DNA, 58 bp, Artificial Sequence
  Description of Artificial Sequence: Synthetic oligonucleotide
ccggccagatatactacaactcaatctcgagattgagttgtagtatatctggtttttg

SEQ ID NO: 628 - DNA, 58 bp, Artificial Sequence
  Description of Artificial Sequence: Synthetic oligonucleotide
aattcaaaaaccagatatactacaactcaatctcgagattgagttgtagtatatctgg

SEQ ID NO: 629 - DNA, 21 bp, Homo sapiens
tgtatgaatgtctgctgggca

SEQ ID NO: 630 - DNA, 58 bp, Artificial Sequence
  Description of Artificial Sequence: Synthetic oligonucleotide
ccggtgtatgaatgtctgctgggcactcgagtgcccagcagacattcatacatttttg

SEQ ID NO: 631 - DNA, 58 bp, Artificial Sequence
  Description of Artificial Sequence: Synthetic oligonucleotide
aattcaaaaatgtatgaatgtctgctgggcactcgagtgcccagcagacattcataca

SEQ ID NO: 632 - DNA, 21 bp, Homo sapiens
caaggacctgaaggagaagaa

SEQ ID NO: 633 - DNA, 58 bp, Artificial Sequence
  Description of Artificial Sequence: Synthetic oligonucleotide
ccggcaaggacctgaaggagaagaactcgagttcttctccttcaggtccttgtttttg

SEQ ID NO: 634 - DNA, 58 bp, Artificial Sequence
  Description of Artificial Sequence: Synthetic oligonucleotide
aattcaaaaacaaggacctgaaggagaagaactcgagttcttctccttcaggtccttg

SEQ ID NO: 635 - DNA, 21 bp, Homo sapiens
caatgtctgtgggaggaagaa

SEQ ID NO: 636 - DNA, 58 bp, Artificial Sequence
  Description of Artificial Sequence: Synthetic oligonucleotide
ccggcaatgtctgtgggaggaagaactcgagttcttcctcccacagacattgtttttg

SEQ ID NO: 637 - DNA, 58 bp, Artificial Sequence
  Description of Artificial Sequence: Synthetic oligonucleotide
aattcaaaaacaatgtctgtgggaggaagaactcgagttcttcctcccacagacattg

SEQ ID NO: 638 - DNA, 21 bp, Homo sapiens
caaggtcctgtagggagaaga

SEQ ID NO: 639 - DNA, 58 bp, Artificial Sequence
  Description of Artificial Sequence: Synthetic oligonucleotide
ccggcaaggtcctgtagggagaagactcgagtcttctccctacaggaccttgtttttg

SEQ ID NO: 640 - DNA, 58 bp, Artificial Sequence
  Description of Artificial Sequence: Synthetic oligonucleotide
aattcaaaaacaaggtcctgtagggagaagactcgagtcttctccctacaggaccttg

SEQ ID NO: 641 - DNA, 21 bp, Homo sapiens
caaggtcctgtagggagaaga

SEQ ID NO: 642 - DNA, 58 bp, Artificial Sequence; identical to SEQ ID NO: 639
  Description of Artificial Sequence: Synthetic oligonucleotide

SEQ ID NO: 643 - DNA, 58 bp, Artificial Sequence; identical to SEQ ID NO: 640
  Description of Artificial Sequence: Synthetic oligonucleotide

SEQ ID NO: 644 - DNA, 21 bp, Homo sapiens
acaagcccaaggtgatcatta

SEQ ID NO: 645 - DNA, 58 bp, Artificial Sequence
  Description of Artificial Sequence: Synthetic oligonucleotide
ccggacaagcccaaggtgatcattactcgagtaatgatcaccttgggcttgttttttg

SEQ ID NO: 646 - DNA, 58 bp, Artificial Sequence
  Description of Artificial Sequence: Synthetic oligonucleotide
aattcaaaaaacaagcccaaggtgatcattactcgagtaatgatcaccttgggcttgt

SEQ ID NO: 647 - DNA, 21 bp, Homo sapiens
caaggacttgaaggagaagaa

SEQ ID NO: 648 - DNA, 58 bp, Artificial Sequence
  Description of Artificial Sequence: Synthetic oligonucleotide
ccggcaaggacttgaaggagaagaactcgagttcttctccttcaagtccttgtttttg

SEQ ID NO: 649 - DNA, 58 bp, Artificial Sequence
  Description of Artificial Sequence: Synthetic oligonucleotide
aattcaaaaacaaggacttgaaggagaagaactcgagttcttctccttcaagtccttg

SEQ ID NO: 650 - DNA, 21 bp, Homo sapiens
cccaagtttgaagtacaagta

SEQ ID NO: 651 - DNA, 58 bp, Artificial Sequence
  Description of Artificial Sequence: Synthetic oligonucleotide
ccggcccaagtttgaagtacaagtactcgagtacttgtacttcaaacttgggtttttg

SEQ ID NO: 652 - DNA, 58 bp, Artificial Sequence
  Description of Artificial Sequence: Synthetic oligonucleotide
aattcaaaaacccaagtttgaagtacaagtactcgagtacttgtacttcaaacttggg

SEQ ID NO: 653 - DNA, 21 bp, Homo sapiens
cccaggacatgataataagat

SEQ ID NO: 654 - DNA, 58 bp, Artificial Sequence
  Description of Artificial Sequence: Synthetic oligonucleotide
ccggcccaggacatgataataagatctcgagatcttattatcatgtcctgggtttttg

SEQ ID NO: 655 - DNA, 58 bp, Artificial Sequence
  Description of Artificial Sequence: Synthetic oligonucleotide
aattcaaaaacccaggacatgataataagatctcgagatcttattatcatgtcctggg

SEQ ID NO: 656 - DNA, 21 bp, Homo sapiens
gaatttgacagtttcctgcca

SEQ ID NO: 657 - DNA, 58 bp, Artificial Sequence
  Description of Artificial Sequence: Synthetic oligonucleotide
ccgggaatttgacagtttcctgccactcgagtggcaggaaactgtcaaattctttttg

SEQ ID NO: 658 - DNA, 58 bp, Artificial Sequence
  Description of Artificial Sequence: Synthetic oligonucleotide
aattcaaaaagaatttgacagtttcctgccactcgagtggcaggaaactgtcaaattc

SEQ ID NO: 659 - DNA, 21 bp, Homo sapiens
cccaagtttgaggtcaaagtt

SEQ ID NO: 660 - DNA, 58 bp, Artificial Sequence
  Description of Artificial Sequence: Synthetic oligonucleotide
ccggcccaagtttgaggtcaaagttctcgagaactttgacctcaaacttgggtttttg

SEQ ID NO: 661 - DNA, 58 bp, Artificial Sequence
  Description of Artificial Sequence: Synthetic oligonucleotide
aattcaaaaacccaagtttgaggtcaaagttctcgagaactttgacctcaaacttggg

SEQ ID NO: 662 - DNA, 21 bp, Homo sapiens
cgacaagatgttctccctcaa

SEQ ID NO: 663 - DNA, 58 bp, Artificial Sequence
  Description of Artificial Sequence: Synthetic oligonucleotide
ccggcgacaagatgttctccctcaactcgagttgagggagaacatcttgtcgtttttg

SEQ ID NO: 664 - DNA, 58 bp, Artificial Sequence
  Description of Artificial Sequence: Synthetic oligonucleotide
aattcaaaaacgacaagatgttctccctcaactcgagttgagggagaacatcttgtcg

SEQ ID NO: 665 - DNA, 21 bp, Artificial Sequence
  Description of Artificial Sequence: Synthetic oligonucleotide
aaaggttaatgtcccgctgaa

SEQ ID NO: 666 - DNA, 21 bp, Homo sapiens
agccactaagtcaagctgaaa

SEQ ID NO: 667 - DNA, 58 bp, Artificial Sequence
  Description of Artificial Sequence: Synthetic oligonucleotide
ccggagccactaagtcaagctgaaactcgagtttcagcttgacttagtggcttttttg

SEQ ID NO: 668 - DNA, 58 bp, Artificial Sequence
  Description of Artificial Sequence: Synthetic oligonucleotide
aattcaaaaaagccactaagtcaagctgaaactcgagtttcagcttgacttagtggct

SEQ ID NO: 669 - DNA, 21 bp, Homo sapiens
ccaactggtctaagcagcatt

SEQ ID NO: 670 - DNA, 58 bp, Artificial Sequence
  Description of Artificial Sequence: Synthetic oligonucleotide
ccggccaactggtctaagcagcattctcgagaatgctgcttagaccagttggtttttg

SEQ ID NO: 671 - DNA, 58 bp, Artificial Sequence
  Description of Artificial Sequence: Synthetic oligonucleotide
aattcaaaaaccaactggtctaagcagcattctcgagaatgctgcttagaccagttgg

SEQ ID NO: 672 - DNA, 21 bp, Homo sapiens
gaaacgaggacacaatgaaat

SEQ ID NO: 673 - DNA, 58 bp, Artificial Sequence
  Description of Artificial Sequence: Synthetic oligonucleotide
ccgggaaacgaggacacaatgaaatctcgagatttcattgtgtcctcgtttctttttg

SEQ ID NO: 674 - DNA, 58 bp, Artificial Sequence
  Description of Artificial Sequence: Synthetic oligonucleotide
aattcaaaaagaaacgaggacacaatgaaatctcgagatttcattgtgtcctcgtttc

SEQ ID NO: 675 - DNA, 21 bp, Homo sapiens
gccactaagtcaagctgaaat

SEQ ID NO: 676 - DNA, 58 bp, Artificial Sequence
  Description of Artificial Sequence: Synthetic oligonucleotide
ccgggccactaagtcaagctgaaatctcgagatttcagcttgacttagtggctttttg

SEQ ID NO: 677 - DNA, 58 bp, Artificial Sequence
  Description of Artificial Sequence: Synthetic oligonucleotide
aattcaaaaagccactaagtcaagctgaaatctcgagatttcagcttgacttagtggc

SEQ ID NO: 678 - DNA, 21 bp, Homo sapiens
ctggagttcatagcaccataa

SEQ ID NO: 679 - DNA, 58 bp, Artificial Sequence
  Description of Artificial Sequence: Synthetic oligonucleotide
ccggctggagttcatagcaccataactcgagttatggtgctatgaactccagtttttg

SEQ ID NO: 680 - DNA, 58 bp, Artificial Sequence
  Description of Artificial Sequence: Synthetic oligonucleotide
aattcaaaaactggagttcatagcaccataactcgagttatggtgctatgaactccag

SEQ ID NO: 681 - DNA, 21 bp, Homo sapiens
cccgctgaacattatcagaaa

SEQ ID NO: 682 - DNA, 58 bp, Artificial Sequence
  Description of Artificial Sequence: Synthetic oligonucleotide
ccggcccgctgaacattatcagaaactcgagtttctgataatgttcagcgggtttttg

SEQ ID NO: 683 - DNA, 58 bp, Artificial Sequence
  Description of Artificial Sequence: Synthetic oligonucleotide
aattcaaaaacccgctgaacattatcagaaactcgagtttctgataatgttcagcggg

SEQ ID NO: 684 - DNA, 21 bp, Homo sapiens
agccactaagtcaagctgaaa

SEQ ID NO: 685 - DNA, 58 bp, Artificial Sequence; identical to SEQ ID NO: 667
  Description of Artificial Sequence: Synthetic oligonucleotide

SEQ ID NO: 686 - DNA, 58 bp, Artificial Sequence; identical to SEQ ID NO: 668
  Description of Artificial Sequence: Synthetic oligonucleotide

SEQ ID NO: 687 - DNA, 21 bp, Homo sapiens
ccaactggtctaagcagcatt

SEQ ID NO: 688 - DNA, 58 bp, Artificial Sequence; identical to SEQ ID NO: 670
  Description of Artificial Sequence: Synthetic oligonucleotide

SEQ ID NO: 689 - DNA, 58 bp, Artificial Sequence; identical to SEQ ID NO: 671
  Description of Artificial Sequence: Synthetic oligonucleotide

SEQ ID NO: 690 - DNA, 21 bp, Homo sapiens
gaaacgaggacacaatgaaat

SEQ ID NO: 691 - DNA, 58 bp, Artificial Sequence; identical to SEQ ID NO: 673
  Description of Artificial Sequence: Synthetic oligonucleotide

SEQ ID NO: 692 - DNA, 58 bp, Artificial Sequence; identical to SEQ ID NO: 674
  Description of Artificial Sequence: Synthetic oligonucleotide

SEQ ID NO: 693 - DNA, 21 bp, Homo sapiens
gccactaagtcaagctgaaat

SEQ ID NO: 694 - DNA, 58 bp, Artificial Sequence; identical to SEQ ID NO: 676
  Description of Artificial Sequence: Synthetic oligonucleotide

SEQ ID NO: 695 - DNA, 58 bp, Artificial Sequence; identical to SEQ ID NO: 677
  Description of Artificial Sequence: Synthetic oligonucleotide

SEQ ID NO: 696 - DNA, 21 bp, Homo sapiens
ctggagttcatagcaccataa

SEQ ID NO: 697 - DNA, 58 bp, Artificial Sequence; identical to SEQ ID NO: 679
  Description of Artificial Sequence: Synthetic oligonucleotide

SEQ ID NO: 698 - DNA, 58 bp, Artificial Sequence; identical to SEQ ID NO: 680
  Description of Artificial Sequence: Synthetic oligonucleotide

SEQ ID NO: 699 - DNA, 21 bp, Homo sapiens
cccgctgaacattatcagaaa

SEQ ID NO: 700 - DNA, 58 bp, Artificial Sequence; identical to SEQ ID NO: 682
  Description of Artificial Sequence: Synthetic oligonucleotide

SEQ ID NO: 701 - DNA, 58 bp, Artificial Sequence; identical to SEQ ID NO: 683
  Description of Artificial Sequence: Synthetic oligonucleotide

SEQ ID NO: 702 - DNA, 21 bp, Homo sapiens
agccactaagtcaagctgaaa

SEQ ID NO: 703 - DNA, 58 bp, Artificial Sequence; identical to SEQ ID NO: 667
  Description of Artificial Sequence: Synthetic oligonucleotide

SEQ ID NO: 704 - DNA, 58 bp, Artificial Sequence; identical to SEQ ID NO: 668
  Description of Artificial Sequence: Synthetic oligonucleotide

SEQ ID NO: 705 - DNA, 21 bp, Homo sapiens
ccaactggtctaagcagcatt

SEQ ID NO: 706 - DNA, 58 bp, Artificial Sequence; identical to SEQ ID NO: 670
  Description of Artificial Sequence: Synthetic oligonucleotide

SEQ ID NO: 707 - DNA, 58 bp, Artificial Sequence; identical to SEQ ID NO: 671
  Description of Artificial Sequence: Synthetic oligonucleotide

SEQ ID NO: 708 - DNA, 21 bp, Homo sapiens
gaaacgaggacacaatgaaat

SEQ ID NO: 709 - DNA, 58 bp, Artificial Sequence; identical to SEQ ID NO: 673
  Description of Artificial Sequence: Synthetic oligonucleotide

SEQ ID NO: 710 - DNA, 58 bp, Artificial Sequence; identical to SEQ ID NO: 674
  Description of Artificial Sequence: Synthetic oligonucleotide

SEQ ID NO: 711 - DNA, 21 bp, Homo sapiens
gccactaagtcaagctgaaat

SEQ ID NO: 712 - DNA, 58 bp, Artificial Sequence; identical to SEQ ID NO: 676
  Description of Artificial Sequence: Synthetic oligonucleotide

SEQ ID NO: 713 - DNA, 58 bp, Artificial Sequence; identical to SEQ ID NO: 677
  Description of Artificial Sequence: Synthetic oligonucleotide

SEQ ID NO: 714 - DNA, 21 bp, Homo sapiens
ctggagttcatagcaccataa

SEQ ID NO: 715 - DNA, 58 bp, Artificial Sequence; identical to SEQ ID NO: 679
  Description of Artificial Sequence: Synthetic oligonucleotide

SEQ ID NO: 716 - DNA, 58 bp, Artificial Sequence; identical to SEQ ID NO: 680
  Description of Artificial Sequence: Synthetic oligonucleotide

SEQ ID NO: 717 - DNA, 21 bp, Homo sapiens
cccgctgaacattatcagaaa

SEQ ID NO: 718 - DNA, 58 bp, Artificial Sequence; identical to SEQ ID NO: 682
  Description of Artificial Sequence: Synthetic oligonucleotide

SEQ ID NO: 719 - DNA, 58 bp, Artificial Sequence; identical to SEQ ID NO: 683
  Description of Artificial Sequence: Synthetic oligonucleotide

SEQ ID NO: 720 - DNA, 21 bp, Homo sapiens
agccactaagtcaagctgaaa

SEQ ID NO: 721 - DNA, 58 bp, Artificial Sequence; identical to SEQ ID NO: 667
  Description of Artificial Sequence: Synthetic oligonucleotide

SEQ ID NO: 722 - DNA, 58 bp, Artificial Sequence; identical to SEQ ID NO: 668
  Description of Artificial Sequence: Synthetic oligonucleotide

SEQ ID NO: 723 - DNA, 21 bp, Homo sapiens
ccaactggtctaagcagcatt

SEQ ID NO: 724 - DNA, 58 bp, Artificial Sequence; identical to SEQ ID NO: 670
  Description of Artificial Sequence: Synthetic oligonucleotide

SEQ ID NO: 725 - DNA, 58 bp, Artificial Sequence; identical to SEQ ID NO: 671
  Description of Artificial Sequence: Synthetic oligonucleotide

SEQ ID NO: 726 - DNA, 21 bp, Homo sapiens
gaaacgaggacacaatgaaat

SEQ ID NO: 727 - DNA, 58 bp, Artificial Sequence; identical to SEQ ID NO: 673
  Description of Artificial Sequence: Synthetic oligonucleotide

SEQ ID NO: 728 - DNA, 58 bp, Artificial Sequence; identical to SEQ ID NO: 674
  Description of Artificial Sequence: Synthetic oligonucleotide

SEQ ID NO: 729 - DNA, 21 bp, Homo sapiens
gccactaagtcaagctgaaat

SEQ ID NO: 730 - DNA, 58 bp, Artificial Sequence; identical to SEQ ID NO: 676
  Description of Artificial Sequence: Synthetic oligonucleotide

SEQ ID NO: 731 - DNA, 58 bp, Artificial Sequence; identical to SEQ ID NO: 677
  Description of Artificial Sequence: Synthetic oligonucleotide

SEQ ID NO: 732 - DNA, 21 bp, Homo sapiens
ctggagttcatagcaccataa

SEQ ID NO: 733 - DNA, 58 bp, Artificial Sequence; identical to SEQ ID NO: 679
  Description of Artificial Sequence: Synthetic oligonucleotide

SEQ ID NO: 734 - DNA, 58 bp, Artificial Sequence; identical to SEQ ID NO: 680
  Description of Artificial Sequence: Synthetic oligonucleotide

SEQ ID NO: 735 - DNA, 21 bp, Homo sapiens
cccgctgaacattatcagaaa

SEQ ID NO: 736 - DNA, 58 bp, Artificial Sequence; identical to SEQ ID NO: 682
  Description of Artificial Sequence: Synthetic oligonucleotide

SEQ ID NO: 737 - DNA, 58 bp, Artificial Sequence; identical to SEQ ID NO: 683
  Description of Artificial Sequence: Synthetic oligonucleotide

SEQ ID NO: 738 - DNA, 21 bp, Homo sapiens
agccactaagtcaagctgaaa

SEQ ID NO: 739 - DNA, 58 bp, Artificial Sequence; identical to SEQ ID NO: 667
  Description of Artificial Sequence: Synthetic oligonucleotide

SEQ ID NO: 740 - DNA, 58 bp, Artificial Sequence; identical to SEQ ID NO: 668
  Description of Artificial Sequence: Synthetic oligonucleotide

SEQ ID NO: 741 - DNA, 21 bp, Homo sapiens
ccaactggtctaagcagcatt

SEQ ID NO: 742 - DNA, 58 bp, Artificial Sequence; identical to SEQ ID NO: 670
  Description of Artificial Sequence: Synthetic oligonucleotide

SEQ ID NO: 743 - DNA, 21 bp, Homo sapiens
gaaacgaggacacaatgaaat

SEQ ID NO: 744 - DNA, 58 bp, Artificial Sequence; identical to SEQ ID NO: 673
  Description of Artificial Sequence: Synthetic oligonucleotide

SEQ ID NO: 745 - DNA, 58 bp, Artificial Sequence; identical to SEQ ID NO: 674
  Description of Artificial Sequence: Synthetic oligonucleotide

SEQ ID NO: 746 - DNA, 21 bp, Homo sapiens
gccactaagtcaagctgaaat

SEQ ID NO: 747 - DNA, 58 bp, Artificial Sequence; identical to SEQ ID NO: 676
  Description of Artificial Sequence: Synthetic oligonucleotide

SEQ ID NO: 748 - DNA, 58 bp, Artificial Sequence; identical to SEQ ID NO: 677
  Description of Artificial Sequence: Synthetic oligonucleotide

SEQ ID NO: 749 - DNA, 21 bp, Homo sapiens
ctggagttcatagcaccataa

SEQ ID NO: 750 - DNA, 58 bp, Artificial Sequence; identical to SEQ ID NO: 679
  Description of Artificial Sequence: Synthetic oligonucleotide

SEQ ID NO: 751 - DNA, 58 bp, Artificial Sequence; identical to SEQ ID NO: 680
  Description of Artificial Sequence: Synthetic oligonucleotide

SEQ ID NO: 752 - DNA, 21 bp, Homo sapiens
cccgctgaacattatcagaaa

SEQ ID NO: 753 - DNA, 58 bp, Artificial Sequence; identical to SEQ ID NO: 682
  Description of Artificial Sequence: Synthetic oligonucleotide

SEQ ID NO: 754 - DNA, 58 bp, Artificial Sequence; identical to SEQ ID NO: 683
  Description of Artificial Sequence: Synthetic oligonucleotide

SEQ ID NO: 755 - DNA, 21 bp, Homo sapiens
gattgtttcaacacaagagta

SEQ ID NO: 756 - DNA, 58 bp, Artificial Sequence
  Description of Artificial Sequence: Synthetic oligonucleotide
ccgggattgtttcaacacaagagtactcgagtactcttgtgttgaaacaatctttttg

SEQ ID NO: 757 - DNA, 58 bp, Artificial Sequence
  Description of Artificial Sequence: Synthetic oligonucleotide
aattcaaaaagattgtttcaacacaagagtactcgagtactcttgtgttgaaacaatc

SEQ ID NO: 758 - DNA, 21 bp, Homo sapiens
agccactaagtcaagctgaaa

SEQ ID NO: 759 - DNA, 58 bp, Artificial Sequence; identical to SEQ ID NO: 667
  Description of Artificial Sequence: Synthetic oligonucleotide

SEQ ID NO: 760 - DNA, 58 bp, Artificial Sequence; identical to SEQ ID NO: 668
  Description of Artificial Sequence: Synthetic oligonucleotide

SEQ ID NO: 761 - DNA, 21 bp, Homo sapiens
ccaactggtctaagcagcatt

SEQ ID NO: 762 - DNA, 58 bp, Artificial Sequence; identical to SEQ ID NO: 670
  Description of Artificial Sequence: Synthetic oligonucleotide

SEQ ID NO: 763 - DNA, 58 bp, Artificial Sequence; identical to SEQ ID NO: 671
  Description of Artificial Sequence: Synthetic oligonucleotide

SEQ ID NO: 764 - DNA, 21 bp, Homo sapiens
gaaacgaggacacaatgaaat

SEQ ID NO: 765 - DNA, 58 bp, Artificial Sequence; identical to SEQ ID NO: 673
  Description of Artificial Sequence: Synthetic oligonucleotide

SEQ ID NO: 766 - DNA, 58 bp, Artificial Sequence; identical to SEQ ID NO: 674
  Description of Artificial Sequence: Synthetic oligonucleotide

SEQ ID NO: 767 - DNA, 21 bp, Homo sapiens
gccactaagtcaagctgaaat

SEQ ID NO: 768 - DNA, 58 bp, Artificial Sequence; identical to SEQ ID NO: 676
  Description of Artificial Sequence: Synthetic oligonucleotide

SEQ ID NO: 769 - DNA, 58 bp, Artificial Sequence; identical to SEQ ID NO: 677
  Description of Artificial Sequence: Synthetic oligonucleotide

SEQ ID NO: 770-779 - skipped (10 entries)

SEQ ID NO: 780 - DNA, 21 bp, Homo sapiens
ctggagttcatagcaccataa

SEQ ID NO: 781 - DNA, 58 bp, Artificial Sequence; identical to SEQ ID NO: 679
  Description of Artificial Sequence: Synthetic oligonucleotide

SEQ ID NO: 782 - DNA, 58 bp, Artificial Sequence; identical to SEQ ID NO: 680
  Description of Artificial Sequence: Synthetic oligonucleotide

SEQ ID NO: 783 - DNA, 21 bp, Homo sapiens
cccgctgaacattatcagaaa

SEQ ID NO: 784 - DNA, 58 bp, Artificial Sequence; identical to SEQ ID NO: 682
  Description of Artificial Sequence: Synthetic oligonucleotide

SEQ ID NO: 785 - DNA, 58 bp, Artificial Sequence; identical to SEQ ID NO: 683
  Description of Artificial Sequence: Synthetic oligonucleotide

SEQ ID NO: 786 - DNA, 21 bp, Homo sapiens
agccactaagtcaagctgaaa

SEQ ID NO: 787 - DNA, 58 bp, Artificial Sequence; identical to SEQ ID NO: 667
  Description of Artificial Sequence: Synthetic oligonucleotide

SEQ ID NO: 788 - DNA, 58 bp, Artificial Sequence; identical to SEQ ID NO: 668
  Description of Artificial Sequence: Synthetic oligonucleotide

SEQ ID NO: 789 - DNA, 21 bp, Homo sapiens
ccaactggtctaagcagcatt

SEQ ID NO: 790 - DNA, 58 bp, Artificial Sequence; identical to SEQ ID NO: 670
  Description of Artificial Sequence: Synthetic oligonucleotide

SEQ ID NO: 791 - DNA, 58 bp, Artificial Sequence; identical to SEQ ID NO: 671
  Description of Artificial Sequence: Synthetic oligonucleotide

SEQ ID NO: 792 - DNA, 21 bp, Homo sapiens
gaaacgaggacacaatgaaat

SEQ ID NO: 793 - DNA, 58 bp, Artificial Sequence; identical to SEQ ID NO: 673
  Description of Artificial Sequence: Synthetic oligonucleotide

SEQ ID NO: 794 - DNA, 58 bp, Artificial Sequence; identical to SEQ ID NO: 674
  Description of Artificial Sequence: Synthetic oligonucleotide

SEQ ID NO: 795 - DNA, 21 bp, Homo sapiens
gccactaagtcaagctgaaat

SEQ ID NO: 796 - DNA, 58 bp, Artificial Sequence; identical to SEQ ID NO: 676
  Description of Artificial Sequence: Synthetic oligonucleotide

SEQ ID NO: 797 - DNA, 58 bp, Artificial Sequence; identical to SEQ ID NO: 677
  Description of Artificial Sequence: Synthetic oligonucleotide

SEQ ID NO: 798 - DNA, 21 bp, Homo sapiens
ctggagttcatagcaccataa

SEQ ID NO: 799 - DNA, 58 bp, Artificial Sequence; identical to SEQ ID NO: 679
  Description of Artificial Sequence: Synthetic oligonucleotide

SEQ ID NO: 800 - DNA, 58 bp, Artificial Sequence; identical to SEQ ID NO: 680
  Description of Artificial Sequence: Synthetic oligonucleotide

SEQ ID NO: 801 - DNA, 21 bp, Homo sapiens
cccgctgaacattatcagaaa

SEQ ID NO: 802 - DNA, 58 bp, Artificial Sequence; identical to SEQ ID NO: 682
  Description of Artificial Sequence: Synthetic oligonucleotide

SEQ ID NO: 803 - DNA, 58 bp, Artificial Sequence; identical to SEQ ID NO: 683
  Description of Artificial Sequence: Synthetic oligonucleotide

SEQ ID NO: 804 - DNA, 6 bp, Artificial Sequence
  Description of Artificial Sequence: Synthetic oligonucleotide
ggttga

SEQ ID NO: 805 - DNA, 4 bp, Artificial Sequence
  Description of Artificial Sequence: Synthetic oligonucleotide
agtt

SEQ ID NO: 806 - DNA, 6 bp, Artificial Sequence
  Description of Artificial Sequence: Synthetic oligonucleotide
ggttgg

SEQ ID NO: 807 - DNA, 6 bp, Artificial Sequence
  Description of Artificial Sequence: Synthetic oligonucleotide
agttgg

SEQ ID NO: 808 - DNA, 6 bp, Artificial Sequence
  Description of Artificial Sequence: Synthetic oligonucleotide
agttga

SEQ ID NO: 809 - DNA, 6 bp, Artificial Sequence
  Description of Artificial Sequence: Synthetic oligonucleotide
rrttrr

SEQ ID NO: 810-863 - skipped (54 entries)

SEQ ID NO: 864 - DNA, 15 bp, Artificial Sequence
  Description of Artificial Sequence: Synthetic oligonucleotide
tcctggaggggttgt

SEQ ID NO: 865-881 - skipped (17 entries)

SEQ ID NO: 882 - protein, 131 aa, Artificial Sequence
  Description of Artificial Sequence: Synthetic polypeptide
MAAPRGRPKKDLTMEDLTAKISQLTVENRELRKALGSTADPRDRPLTATEKEAQLTATVGALSAAAAKKIEARVRTIFSKVVTQKQVDDALKGLSLRIDVCMSDGGTAKPPPGANNRRRRGASTTRAGVDD

SEQ ID NO: 883 - protein, 1129 aa, Artificial Sequence
  Description of Artificial Sequence: Synthetic polypeptide
MAPPGMRLRSGRSTGAPLTRGSCRKRNRSPERCDLGDDLHLQPRRKHVADSVDGRECGPHTLPIPGSPTVFTSGLPAFVSSPTLPVAPIPSPAPATPLPPPALLPPVTTSSSPIPPSHPVSPGTTDTHSPSPALPPTQSPESSQRPPLSSPTGRPDSSTPMRPPPSQQTTPPHSPTTPPPEPPSKSSPDSLAPSTLRSLRKRRLSSPQGPSTLNPICQSPPVSPPRCDFANRSVYPPWATESPIYVGSSSDGDTPPRQPPTSPISIGSSSPSEGSWGDDTAMLVLLAEIAEEASKNEKECSENNQAGEDNGDNEISKESQVDKDDNDNKDDEEEQETDEEDEEDDEEDDEEDDEEDDEEDDEEDDEEDDEEEDEEEDEEEDEEEDEEEEEDEEDDDDEDNEDEEDDEEEDKKEDEEDGGDGNKTLSIQSSQQQQEPQQQEPQQQEPQQQEPQQQEPQQQEPQQQEPQQQEPQQREPQQREPQQREPQQREPQQREPQQREPQQREPQQREPQQREPQQREPQQREPQQREPQQQEPQQQEPQQQEPQQQEPQQQEPQQQEPQQQEPQQQEPQQQEPQQQEPQQQEPQQQEPQQQDEQQQDEQQQDEQQQDEQQQDEQQQDEQQQDEQQQDEQEQQDEQQQDEQQQQDEQEQQEEQEQQEEQQQDEQQQDEQQQDEQQQDEQEQQDEQQQDEQQQQDEQEQQEEQEQQEEQEQQEEQEQQEEQEQELEEQEQELEEQEQELEEQEQELEEQEQELEEQEQELEEQEQELEEQEQELEEQEQELEEQEQELEEQEQELEEQEQELEEQEQELEEQEQELEEQEQEQELEEVEEQEQEQEEQELEEVEEQEQEQEEQEEQELEEVEEQEEQELEEVEEQEEQELEEVEEQEQQGVEQQEQETVEEPIILHGSSSEDEMEVDYPVVSTHEQIASSPPGDNTPDDDPQPGPSREYRYVLRTSPPHRPGVRMRRVPVTHPKKPHPRYQQPPVPYRQIDDCPAKARPQHIFYRRFLGKDGRRDPKCQWKFAVIFWGNDPYGLKKLSQAFQFGGVKAGPVSCLPHPGPDQSPITYCVYVYCQNKDTSKKVQMARLAWEASHPLAGNLQSSIVKFKKPLPLTQPGENQGPGDSPQEMT

SEQ ID NO: 884 - protein, 160 aa, Artificial Sequence
  Description of Artificial Sequence: Synthetic polypeptide
MAPPGMRLRSGRSTGAPLTRGSCRKRNRSPERCDLGDDLHLQPRRKHVADSVDGRECGPHTLPIPGSPTVFTSGLPAFVSSPTLPVAPIPSPAPATPLPPPALLPPVTTSSSPIPPSHPVSPGTTDTHSPSPALPPTQSPESSQRPPLSSPTGRPDSSTP

SEQ ID NO: 885 - DNA, 84 bp, Artificial Sequence
  Description of Artificial Sequence: Synthetic oligonucleotide
ggccagtgttgagaggcggagacttgggcaattgctggacgctgccctgggcattgcacttgtctcggtctgacagtgccggcc

SEQ ID NO: 886 - DNA, 80 bp, Artificial Sequence
  Description of Artificial Sequence: Synthetic oligonucleotide
ctgggggctccaaagtgctgttcgtgcaggtagtgtgattacccaacctactgctgagctagcacttcccgagcccccgg

SEQ ID NO: 887 - DNA, 15 bp, Artificial Sequence
  Description of Artificial Sequence: Synthetic oligonucleotide
  modified_base (4)..(8): a, c, t, g, unknown or other
  misc_feature (4)..(8): This region may encompass 3-5 nucleotides
  misc_feature (9)..(13): This region may encompass 3-5 nucleotides
cctnnnnngggggrr

SEQ ID NO: 888 - DNA, 6 bp, Artificial Sequence
  Description of Artificial Sequence: Synthetic oligonucleotide
ttaggg

SEQ ID NO: 889 - DNA, 15 bp, Artificial Sequence
  Description of Artificial Sequence: Synthetic oligonucleotide
tcctggcggggaagt

SEQ ID NO: 890 - DNA, 15 bp, Artificial Sequence
  Description of Artificial Sequence: Synthetic oligonucleotide
tcctggaggggaagt

SEQ ID NO: 891 - DNA, 20 bp, Artificial Sequence
  Description of Artificial Sequence: Synthetic oligonucleotide
gggggggggggggggggggg

SEQ ID NO: 892 - DNA, 24 bp, Artificial Sequence
  Description of Artificial Sequence: Synthetic oligonucleotide
ttagggttagggttagggttaggg

SEQ ID NO: 893 - DNA, 21 bp, Artificial Sequence
  Description of Artificial Sequence: Synthetic oligonucleotide
ctcctattgggggtttcctat

SEQ ID NO: 894 - DNA, 15 bp, Artificial Sequence
  Description of Artificial Sequence: Synthetic oligonucleotide
tcctggaggggttgt

SEQ ID NO: 895 - DNA, 24 bp, Artificial Sequence
  Description of Artificial Sequence: Synthetic oligonucleotide
cctggatgggaattcccatccagg

SEQ ID NO: 896 - DNA, 24 bp, Artificial Sequence
  Description of Artificial Sequence: Synthetic oligonucleotide
ttcccatccaggcctggatgggaa

SEQ ID NO: 897 - DNA, 20 bp, Artificial Sequence
  Description of Artificial Sequence: Synthetic oligonucleotide
tgcttgcaagcttgcaagca

SEQ ID NO: 898 - DNA, 15 bp, Artificial Sequence
  Description of Artificial Sequence: Synthetic oligonucleotide
tcctggatgggaagt

SEQ ID NO: 899 - DNA, 12 bp, Artificial Sequence
  Description of Artificial Sequence: Synthetic oligonucleotide
cctggatgggaa

SEQ ID NO: 900 - DNA, 24 bp, Artificial Sequence
  Description of Artificial Sequence: Synthetic oligonucleotide
cttaccgctgcacctggatgggaa

SEQ ID NO: 901 - DNA, 24 bp, Artificial Sequence
  Description of Artificial Sequence: Synthetic oligonucleotide
cctggatgggaacttaccgctgca

SEQ ID NO: 902 - DNA, 18 bp, Artificial Sequence
  Description of Artificial Sequence: Synthetic oligonucleotide
tgctcctggaggggttgt

SEQ ID NO: 903 - DNA, 26 bp, Artificial Sequence
  Description of Artificial Sequence: Synthetic oligonucleotide
ggtggtggtggttgtggtggtggtgg

SEQ ID NO: 904 - RNA, 19 nt, Artificial Sequence
  Description of Artificial Sequence: Synthetic oligonucleotide
gaaggcccauauagagaaa

SEQ ID NO: 905 - DNA, 143 bp, Artificial Sequence
  Description of Artificial Sequence: Synthetic polynucleotide
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 906 - DNA, 143 bp, Artificial Sequence
  Description of Artificial Sequence: Synthetic polynucleotide
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 907 - DNA, 141 bp, Artificial Sequence; identical to SEQ ID NO: 51
  Description of Artificial Sequence: Synthetic polynucleotide

SEQ ID NO: 908 - DNA, 141 bp, Artificial Sequence; identical to SEQ ID NO: 1
  Description of Artificial Sequence: Synthetic polynucleotide

SEQ ID NO: 909 - DNA, 144 bp, Artificial Sequence
  Description of Artificial Sequence: Synthetic polynucleotide
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 910 - DNA, 144 bp, Artificial Sequence
  Description of Artificial Sequence: Synthetic polynucleotide
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 911 - DNA, 127 bp, Artificial Sequence
  Description of Artificial Sequence: Synthetic polynucleotide
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 912 - DNA, 127 bp, Artificial Sequence
  Description of Artificial Sequence: Synthetic polynucleotide
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 913 - DNA, 166 bp, Artificial Sequence
  Description of Artificial Sequence: Synthetic polynucleotide
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 914 - DNA, 166 bp, Artificial Sequence
  Description of Artificial Sequence: Synthetic polynucleotide
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 915 - DNA, 144 bp, Artificial Sequence
  Description of Artificial Sequence: Synthetic polynucleotide
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 916 - DNA, 144 bp, Artificial Sequence
  Description of Artificial Sequence: Synthetic polynucleotide
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 917 - DNA, 120 bp, Artificial Sequence
  Description of Artificial Sequence: Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 918 - DNA, 122 bp, Artificial Sequence
  Description of Artificial Sequence: Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 919 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 920 - DNA, 101 bp, Artificial Sequence
  Description of Artificial Sequence: Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 921 - DNA, 139 bp, Artificial Sequence
  Description of Artificial Sequence: Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 922 - DNA, 137 bp, Artificial Sequence
  Description of Artificial Sequence: Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 923 - DNA, 135 bp, Artificial Sequence
  Description of Artificial Sequence: Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 924 - DNA, 133 bp, Artificial Sequence
  Description of Artificial Sequence: Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 925 - DNA, 139 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 926 - DNA, 137 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 927 - DNA, 135 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 928 - DNA, 133 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 929 - DNA, 131 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 930 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 931 - DNA, 127 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 932 - DNA, 122 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 933 - DNA, 130 bp, Artificial Sequence; identical to SEQ ID NO: 2
  Description of Artificial Sequence: Synthetic polynucleotide

SEQ ID NO: 934 - DNA, 120 bp, Artificial Sequence
  Description of Artificial Sequence: Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 935 - DNA, 122 bp, Artificial Sequence
  Description of Artificial Sequence: Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 936 - DNA, 122 bp, Artificial Sequence
  Description of Artificial Sequence: Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 937 - DNA, 129 bp, Artificial Sequence
  Description of Artificial Sequence: Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 938 - DNA, 101 bp, Artificial Sequence
  Description of Artificial Sequence: Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 939 - DNA, 139 bp, Artificial Sequence
  Description of Artificial Sequence: Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 940 - DNA, 137 bp, Artificial Sequence
  Description of Artificial Sequence: Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 941 - DNA, 135 bp, Artificial Sequence
  Description of Artificial Sequence: Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 942 - DNA, 133 bp, Artificial Sequence
  Description of Artificial Sequence: Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 943 - DNA, 139 bp, Artificial Sequence
  Description of Artificial Sequence: Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 944 - DNA, 137 bp, Artificial Sequence
  Description of Artificial Sequence: Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 945 - DNA, 135 bp, Artificial Sequence
  Description of Artificial Sequence: Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 946 - DNA, 133 bp, Artificial Sequence
  Description of Artificial Sequence: Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 947 - DNA, 131 bp, Artificial Sequence
  Description of Artificial Sequence: Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 948 - DNA, 129 bp, Artificial Sequence
  Description of Artificial Sequence: Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 949 - DNA, 127 bp, Artificial Sequence
  Description of Artificial Sequence: Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 950 - protein, 19 aa, Artificial Sequence
  Description of Artificial Sequence: Synthetic peptide
MDWTWRILFLVAAATGAHS

SEQ ID NO: 951 - protein, 19 aa, Artificial Sequence
  Description of Artificial Sequence: Synthetic peptide
MLPSQLIGFLLLWVPASRG

SEQ ID NO: 952 - DNA, 141 bp, Artificial Sequence; identical to SEQ ID NO: 1
  Description of Artificial Sequence: Synthetic polynucleotide

SEQ ID NO: 953 - DNA, 141 bp, Artificial Sequence; identical to SEQ ID NO: 51
  Description of Artificial Sequence: Synthetic polynucleotide

SEQ ID NO: 954 - DNA, 130 bp, Artificial Sequence; identical to SEQ ID NO: 2
  Description of Artificial Sequence: Synthetic polynucleotide

SEQ ID NO: 955 - DNA, 130 bp, Artificial Sequence; identical to SEQ ID NO: 52
  Description of Artificial Sequence: Synthetic polynucleotide

SEQ ID NO: 956 - DNA, 143 bp, Artificial Sequence; identical to SEQ ID NO: 905
  Description of Artificial Sequence: Synthetic polynucleotide

SEQ ID NO: 957 - DNA, 144 bp, Artificial Sequence; identical to SEQ ID NO: 909
  Description of Artificial Sequence: Synthetic polynucleotide

SEQ ID NO: 958 - DNA, 127 bp, Artificial Sequence; identical to SEQ ID NO: 911
  Description of Artificial Sequence: Synthetic polynucleotide

SEQ ID NO: 959 - DNA, 166 bp, Artificial Sequence; identical to SEQ ID NO: 913
  Description of Artificial Sequence: Synthetic polynucleotide

SEQ ID NO: 960 - DNA, 144 bp, Artificial Sequence; identical to SEQ ID NO: 915
  Description of Artificial Sequence: Synthetic polynucleotide

SEQ ID NO: 961 - DNA, 143 bp, Artificial Sequence; identical to SEQ ID NO: 906
  Description of Artificial Sequence: Synthetic polynucleotide

SEQ ID NO: 962 - DNA, 144 bp, Artificial Sequence; identical to SEQ ID NO: 910
  Description of Artificial Sequence: Synthetic polynucleotide

SEQ ID NO: 963 - DNA, 127 bp, Artificial Sequence; identical to SEQ ID NO: 912
  Description of Artificial Sequence: Synthetic polynucleotide

SEQ ID NO: 964 - DNA, 166 bp, Artificial Sequence; identical to SEQ ID NO: 914
  Description of Artificial Sequence: Synthetic polynucleotide

SEQ ID NO: 965 - DNA, 144 bp, Artificial Sequence; identical to SEQ ID NO: 916
  Description of Artificial Sequence: Synthetic polynucleotide

SEQ ID NO: 966 - DNA, 120 bp, Artificial Sequence; identical to SEQ ID NO: 917
  Description of Artificial Sequence: Synthetic polynucleotide

SEQ ID NO: 967 - DNA, 122 bp, Artificial Sequence; identical to SEQ ID NO: 918
  Description of Artificial Sequence: Synthetic polynucleotide

SEQ ID NO: 968 - DNA, 129 bp, Artificial Sequence; identical to SEQ ID NO: 919
  Description of Artificial Sequence: Synthetic polynucleotide

SEQ ID NO: 969 - DNA, 101 bp, Artificial Sequence; identical to SEQ ID NO: 920
  Description of Artificial Sequence: Synthetic polynucleotide

SEQ ID NO: 970 - DNA, 139 bp, Artificial Sequence; identical to SEQ ID NO: 921
  Description of Artificial Sequence: Synthetic polynucleotide

SEQ ID NO: 971 - DNA, 137 bp, Artificial Sequence; identical to SEQ ID NO: 922
  Description of Artificial Sequence: Synthetic polynucleotide

SEQ ID NO: 972 - DNA, 135 bp, Artificial Sequence; identical to SEQ ID NO: 923
  Description of Artificial Sequence: Synthetic polynucleotide

SEQ ID NO: 973 - DNA, 133 bp, Artificial Sequence; identical to SEQ ID NO: 924
  Description of Artificial Sequence: Synthetic polynucleotide

SEQ ID NO: 974 - DNA, 139 bp, Artificial Sequence; identical to SEQ ID NO: 925
  Description of Artificial Sequence: Synthetic polynucleotide

SEQ ID NO: 975 - DNA, 137 bp, Artificial Sequence; identical to SEQ ID NO: 926
  Description of Artificial Sequence: Synthetic polynucleotide

SEQ ID NO: 976 - DNA, 135 bp, Artificial Sequence; identical to SEQ ID NO: 927
  Description of Artificial Sequence: Synthetic polynucleotide

SEQ ID NO: 977 - DNA, 133 bp, Artificial Sequence; identical to SEQ ID NO: 928
  Description of Artificial Sequence: Synthetic polynucleotide

SEQ ID NO: 978 - DNA, 131 bp, Artificial Sequence; identical to SEQ ID NO: 929
  Description of Artificial Sequence: Synthetic polynucleotide

SEQ ID NO: 979 - DNA, 129 bp, Artificial Sequence; identical to SEQ ID NO: 930
  Description of Artificial Sequence: Synthetic polynucleotide

SEQ ID NO: 980 - DNA, 127 bp, Artificial Sequence; identical to SEQ ID NO: 931
  Description of Artificial Sequence: Synthetic polynucleotide

SEQ ID NO: 981 - DNA, 122 bp, Artificial Sequence; identical to SEQ ID NO: 932
  Description of Artificial Sequence: Synthetic polynucleotide

SEQ ID NO: 982 - DNA, 130 bp, Artificial Sequence; identical to SEQ ID NO: 2
  Description of Artificial Sequence: Synthetic polynucleotide

SEQ ID NO: 983 - DNA, 120 bp, Artificial Sequence; identical to SEQ ID NO: 934
  Description of Artificial Sequence: Synthetic polynucleotide

SEQ ID NO: 984 - DNA, 122 bp, Artificial Sequence; identical to SEQ ID NO: 935
  Description of Artificial Sequence: Synthetic polynucleotide

SEQ ID NO: 985 - DNA, 122 bp, Artificial Sequence; identical to SEQ ID NO: 936
  Description of Artificial Sequence: Synthetic polynucleotide

SEQ ID NO: 986 - DNA, 129 bp, Artificial Sequence; identical to SEQ ID NO: 937
  Description of Artificial Sequence: Synthetic polynucleotide

SEQ ID NO: 987 - DNA, 101 bp, Artificial Sequence; identical to SEQ ID NO: 938
  Description of Artificial Sequence: Synthetic polynucleotide

SEQ ID NO: 988 - DNA, 139 bp, Artificial Sequence; identical to SEQ ID NO: 939
  Description of Artificial Sequence: Synthetic polynucleotide

SEQ ID NO: 989 - DNA, 137 bp, Artificial Sequence; identical to SEQ ID NO: 940
  Description of Artificial Sequence: Synthetic polynucleotide

SEQ ID NO: 990 - DNA, 135 bp, Artificial Sequence; identical to SEQ ID NO: 941
  Description of Artificial Sequence: Synthetic polynucleotide

SEQ ID NO: 991 - DNA, 133 bp, Artificial Sequence; identical to SEQ ID NO: 942
  Description of Artificial Sequence: Synthetic polynucleotide

SEQ ID NO: 992 - DNA, 139 bp, Artificial Sequence; identical to SEQ ID NO: 943
  Description of Artificial Sequence: Synthetic polynucleotide

SEQ ID NO: 993 - DNA, 137 bp, Artificial Sequence; identical to SEQ ID NO: 944
  Description of Artificial Sequence: Synthetic polynucleotide

SEQ ID NO: 994 - DNA, 135 bp, Artificial Sequence; identical to SEQ ID NO: 945
  Description of Artificial Sequence: Synthetic polynucleotide

SEQ ID NO: 995 - DNA, 133 bp, Artificial Sequence; identical to SEQ ID NO: 946
  Description of Artificial Sequence: Synthetic polynucleotide

SEQ ID NO: 996 - DNA, 131 bp, Artificial Sequence; identical to SEQ ID NO: 947
  Description of Artificial Sequence: Synthetic polynucleotide

SEQ ID NO: 997 - DNA, 129 bp, Artificial Sequence; identical to SEQ ID NO: 948
  Description of Artificial Sequence: Synthetic polynucleotide

SEQ ID NO: 998 - DNA, 127 bp, Artificial Sequence; identical to SEQ ID NO: 949
  Description of Artificial Sequence: Synthetic polynucleotide

SEQ ID NO: 999 - DNA, 122 bp, Artificial Sequence; identical to SEQ ID NO: 932
  Description of Artificial Sequence: Synthetic polynucleotide

SEQ ID NO: 1000 - DNA, 12 bp, Artificial Sequence
  Description of Artificial Sequence: Synthetic oligonucleotide
cgatcgttcgat

SEQ ID NO: 1001 - DNA, 12 bp, Artificial Sequence
  Description of Artificial Sequence: Synthetic oligonucleotide
atcgaaccatcg

SEQ ID NO: 1002 - DNA, 12 bp, Artificial Sequence
  Description of Artificial Sequence: Synthetic oligonucleotide
atcgaacgatcg

SEQ ID NO: 1003 - DNA, 165 bp, Artificial Sequence
  Description of Artificial Sequence: Synthetic polynucleotide
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 1004 - DNA, 140 bp, Artificial Sequence
  Description of Artificial Sequence: Synthetic polynucleotide
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 1005 - DNA, 91 bp, Artificial Sequence
  Description of Artificial Sequence: Synthetic oligonucleotide
gcgcgctcgctcgctcactgaggccgcccgggcaaagcccgggcgtcgggcgacctttggtcgcccggcctcagtgagcgagcgagcgcgc

SEQ ID NO: 1006 - DNA, 91 bp, Artificial Sequence
  Description of Artificial Sequence: Synthetic oligonucleotide
gcgcgctcgctcgctcactgaggccgggcgaccaaaggtcgcccgacgcccgggctttgcccgggcggcctcagtgagcgagcgagcgcgc

SEQ ID NO: 1007 - DNA, 1662 bp, Artificial Sequence; identical to SEQ ID NO: 71
  Description of Artificial Sequence: Synthetic polynucleotide

SEQ ID NO: 1008 - protein, 453 aa, Artificial Sequence
  Description of Artificial Sequence: Synthetic polypeptide
  MOD_RES (1)..(1): Any amino acid
XVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 1009 - protein, 214 aa, Artificial Sequence
  Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1010 - DNA, 1310 bp, Artificial Sequence
  Description of Artificial Sequence: Synthetic polynucleotide
ggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaa

SEQ ID NO: 1011 - DNA, 16 bp, Artificial Sequence
  Description of Artificial Sequence: Synthetic oligonucleotide
gcgcgctcgctcgctc

SEQ ID NO: 1012 - DNA, 6 bp, Artificial Sequence
  Description of Artificial Sequence: Synthetic oligonucleotide
ggttga

SEQ ID NO: 1013 - DNA, 4 bp, Artificial Sequence
  Description of Artificial Sequence: Synthetic oligonucleotide
agtt

SEQ ID NO: 1014 - DNA, 6 bp, Artificial Sequence
  Description of Artificial Sequence: Synthetic oligonucleotide
ggttgg

SEQ ID NO: 1015 - DNA, 6 bp, Artificial Sequence
  Description of Artificial Sequence: Synthetic oligonucleotide
agttgg

SEQ ID NO: 1016 - DNA, 6 bp, Artificial Sequence
  Description of Artificial Sequence: Synthetic oligonucleotide
agttga

SEQ ID NO: 1017 - DNA, 6 bp, Artificial Sequence
  Description of Artificial Sequence: Synthetic oligonucleotide
rrttrr

SEQ ID NO: 1018 - DNA, 581 bp, Artificial Sequence; identical to SEQ ID NO: 8
  Description of Artificial Sequence: Synthetic polynucleotide

SEQ ID NO: 1019 - DNA, 225 bp, Artificial Sequence; identical to SEQ ID NO: 9
  Description of Artificial Sequence: Synthetic polynucleotide

SEQ ID NO: 1020 - DNA, 8 bp, Artificial Sequence
  Description of Artificial Sequence: Synthetic oligonucleotide
actgaggc

SEQ ID NO: 1021 - DNA, 8 bp, Artificial Sequence
  Description of Artificial Sequence: Synthetic oligonucleotide
gcctcagt

SEQ ID NO: 1022 - DNA, 16 bp, Artificial Sequence
  Description of Artificial Sequence: Synthetic oligonucleotide
gagcgagcgagcgcgc

SEQ ID NO: 1023 - DNA, 1923 bp, Artificial Sequence; identical to SEQ ID NO: 3
  Description of Artificial Sequence: Synthetic polynucleotide

SEQ ID NO: 1024 - DNA, 1272 bp, Artificial Sequence; identical to SEQ ID NO: 4
  Description of Artificial Sequence: Synthetic polynucleotide

SEQ ID NO: 1025 - DNA, 1177 bp, Artificial Sequence; identical to SEQ ID NO: 74
  Description of Artificial Sequence: Synthetic polynucleotide

SEQ ID NO: 1026 - DNA, 547 bp, Artificial Sequence; identical to SEQ ID NO: 5
  Description of Artificial Sequence: Synthetic polynucleotide

SEQ ID NO: 1027 - DNA, 556 bp, Artificial Sequence; identical to SEQ ID NO: 16
  Description of Artificial Sequence: Synthetic polynucleotide

SEQ ID NO: 1028 - DNA, 1179 bp, Artificial Sequence; identical to SEQ ID NO: 6
  Description of Artificial Sequence: Synthetic polynucleotide

SEQ ID NO: 1029 - DNA, 141 bp, Artificial Sequence; identical to SEQ ID NO: 15
  Description of Artificial Sequence: Synthetic polynucleotide

SEQ ID NO: 1030 - DNA, 317 bp, Artificial Sequence; identical to SEQ ID NO: 308
  Description of Artificial Sequence: Synthetic polynucleotide

SEQ ID NO: 1031 - DNA, 241 bp, Artificial Sequence; identical to SEQ ID NO: 18
  Description of Artificial Sequence: Synthetic polynucleotide

SEQ ID NO: 1032 - DNA, 215 bp, Artificial Sequence; identical to SEQ ID NO: 19
  Description of Artificial Sequence: Synthetic polynucleotide

SEQ ID NO: 1033 - DNA, 546 bp, Artificial Sequence; identical to SEQ ID NO: 135
  Description of Artificial Sequence: Synthetic polynucleotide

SEQ ID NO: 1034 - DNA, 576 bp, Artificial Sequence; identical to SEQ ID NO: 309
  Description of Artificial Sequence: Synthetic polynucleotide

SEQ ID NO: 1035 - DNA, 150 bp, Artificial Sequence; identical to SEQ ID NO: 20
  Description of Artificial Sequence: Synthetic polynucleotide

SEQ ID NO: 1036 - DNA, 1313 bp, Artificial Sequence; identical to SEQ ID NO: 310
  Description of Artificial Sequence: Synthetic polynucleotide

SEQ ID NO: 1037 - DNA, 213 bp, Artificial Sequence; identical to SEQ ID NO: 10
  Description of Artificial Sequence: Synthetic polynucleotide

SEQ ID NO: 1038 - protein, 7 aa, Artificial Sequence
  Description of Artificial Sequence: Synthetic peptide
PKKKRKV

SEQ ID NO: 1039 - protein, 19 aa, Artificial Sequence
  Description of Artificial Sequence: Synthetic peptide
MDWTWRILFLVAAATGAHS

SEQ ID NO: 1040 - protein, 19 aa, Artificial Sequence
  Description of Artificial Sequence: Synthetic peptide
MLPSQLIGFLLLWVPASRG

SEQ ID NO: 1041 - protein, 7 aa, Simian virus 40
PKKKRKV

SEQ ID NO: 1042 - DNA, 21 bp, Simian virus 40
cccaagaagaagaggaaggtg

SEQ ID NO: 1043 - protein, 16 aa, Unknown
  Description of Unknown: Nucleoplasmin bipartite NLS sequence
KRPAATKKAGQAKKKK

SEQ ID NO: 1044 - protein, 9 aa, Unknown
  Description of Unknown: C-myc NLS sequence
PAAKRVKLD

SEQ ID NO: 1045 - protein, 11 aa, Unknown
  Description of Unknown: C-myc NLS sequence
RQRRNELKRSP

SEQ ID NO: 1046 - protein, 38 aa, Homo sapiens; identical to SEQ ID NO: 319

SEQ ID NO: 1047 - protein, 42 aa, Unknown; identical to SEQ ID NO: 320
  Description of Unknown: IBB domain from importin-alpha sequence

SEQ ID NO: 1048 - protein, 8 aa, Unknown
  Description of Unknown: Myoma T protein sequence
VSRKRPRP

SEQ ID NO: 1049 - protein, 8 aa, Unknown
  Description of Unknown: Myoma T protein sequence
PPKKARED

SEQ ID NO: 1050 - protein, 8 aa, Homo sapiens
PQPKKKPL

SEQ ID NO: 1051 - protein, 12 aa, Mus musculus
SALIKKKKKMAP

SEQ ID NO: 1052 - DNA, 70 bp, Artificial Sequence; identical to SEQ ID NO: 101
  Description of Artificial Sequence: Synthetic oligonucleotide

SEQ ID NO: 1053 - DNA, 70 bp, Artificial Sequence; identical to SEQ ID NO: 102
  Description of Artificial Sequence: Synthetic oligonucleotide

SEQ ID NO: 1054 - DNA, 72 bp, Artificial Sequence; identical to SEQ ID NO: 103
  Description of Artificial Sequence: Synthetic oligonucleotide

SEQ ID NO: 1055 - DNA, 72 bp, Artificial Sequence; identical to SEQ ID NO: 104
  Description of Artificial Sequence: Synthetic oligonucleotide

SEQ ID NO: 1056 - DNA, 72 bp, Artificial Sequence; identical to SEQ ID NO: 105
  Description of Artificial Sequence: Synthetic oligonucleotide

SEQ ID NO: 1057 - DNA, 72 bp, Artificial Sequence; identical to SEQ ID NO: 106
  Description of Artificial Sequence: Synthetic oligonucleotide

SEQ ID NO: 1058 - DNA, 83 bp, Artificial Sequence; identical to SEQ ID NO: 107
  Description of Artificial Sequence: Synthetic oligonucleotide

SEQ ID NO: 1059 - DNA, 83 bp, Artificial Sequence; identical to SEQ ID NO: 108
  Description of Artificial Sequence: Synthetic oligonucleotide

SEQ ID NO: 1060 - DNA, 77 bp, Artificial Sequence; identical to SEQ ID NO: 109
  Description of Artificial Sequence: Synthetic oligonucleotide

SEQ ID NO: 1061 - DNA, 77 bp, Artificial Sequence; identical to SEQ ID NO: 110
  Description of Artificial Sequence: Synthetic oligonucleotide

SEQ ID NO: 1062 - DNA, 51 bp, Artificial Sequence; identical to SEQ ID NO: 111
  Description of Artificial Sequence: Synthetic oligonucleotide

SEQ ID NO: 1063 - DNA, 51 bp, Artificial Sequence; identical to SEQ ID NO: 112
  Description of Artificial Sequence: Synthetic oligonucleotide

SEQ ID NO: 1064 - DNA, 80 bp, Artificial Sequence; identical to SEQ ID NO: 65
  Description of Artificial Sequence: Synthetic oligonucleotide

SEQ ID NO: 1065 - DNA, 80 bp, Artificial Sequence; identical to SEQ ID NO: 17
  Description of Artificial Sequence: Synthetic oligonucleotide

SEQ ID NO: 1066 - DNA, 79 bp, Artificial Sequence; identical to SEQ ID NO: 115
  Description of Artificial Sequence: Synthetic oligonucleotide

SEQ ID NO: 1067 - DNA, 79 bp, Artificial Sequence; identical to SEQ ID NO: 116
  Description of Artificial Sequence: Synthetic oligonucleotide

SEQ ID NO: 1068 - protein, 5 aa, Influenza virus
DRLRR

SEQ ID NO: 1069 - protein, 7 aa, Influenza virus
PKQKKRK

SEQ ID NO: 1070 - protein, 10 aa, Hepatitis delta virus
RKLKKKIKKL

SEQ ID NO: 1071 - protein, 10 aa, Mus musculus
REKKKFLKRR

SEQ ID NO: 1072 - protein, 20 aa, Homo sapiens
KRKGDEVDGVDEVAKKKSKK

SEQ ID NO: 1073 - protein, 17 aa, Homo sapiens
RKCLQAGMNLEARKTKK

SEQ ID NO: 1074 - DNA, 8 bp, Artificial Sequence
  Description of Artificial Sequence: Synthetic oligonucleotide
gtttaaac

SEQ ID NO: 1075 - DNA, 8 bp, Artificial Sequence
  Description of Artificial Sequence: Synthetic oligonucleotide
ttaattaa

SEQ ID NO: 1076 - DNA, 141 bp, Artificial Sequence; identical to SEQ ID NO: 15
  Description of Artificial Sequence: Synthetic polynucleotide

SEQ ID NO: 1077 - DNA, 317 bp, Artificial Sequence; identical to SEQ ID NO: 308
  Description of Artificial Sequence: Synthetic polynucleotide

SEQ ID NO: 1078 - DNA, 72 bp, Artificial Sequence; identical to SEQ ID NO: 104
  Description of Artificial Sequence: Synthetic oligonucleotide

SEQ ID NO: 1079 - DNA, 60 bp, Artificial Sequence
  Description of Artificial Sequence: Synthetic oligonucleotide
gagacagacacactcctgctatgggtactgctgctctgggttccaggttccactggtgac

SEQ ID NO: 1080 - DNA, 1260 bp, Adeno-associated virus - 2
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 1081 - DNA, 1932 bp, Adeno-associated virus - 2
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctga

SEQ ID NO: 1082 - DNA, 1876 bp, Artificial Sequence; identical to SEQ ID NO: 13
  Description of Artificial Sequence: Synthetic polynucleotide

SEQ ID NO: 1083 - DNA, 1194 bp, Artificial Sequence; identical to SEQ ID NO: 14
  Description of Artificial Sequence: Synthetic polynucleotide

SEQ ID NO: 1084 - DNA, 1876 bp, Artificial Sequence; identical to SEQ ID NO: 13
  Description of Artificial Sequence: Synthetic polynucleotide

SEQ ID NO: 1085 - DNA, 51 bp, Artificial Sequence
  Description of Artificial Sequence: Synthetic oligonucleotide
ctaggccgcccgggcaaagcccgggcgtcgggcgacctttggtcgcccggc

SEQ ID NO: 1086 - DNA, 65 bp, Artificial Sequence
  Description of Artificial Sequence: Synthetic oligonucleotide
ctaggactgaggccgcccgggcaaagcccgggcgtcgggcgacctttggtcgcccggcctcagtc

SEQ ID NO: 1087 - DNA, 67 bp, Artificial Sequence
  Description of Artificial Sequence: Synthetic oligonucleotide
ggactgaggccgcccgggcaaagcccgggcgtcgggcgacctttggtcgcccggcctcagtcctgca

SEQ ID NO: 1088 - DNA, 41 bp, Artificial Sequence
  Description of Artificial Sequence: Synthetic oligonucleotide
gtgcgggcgaccaaaggtcgcccgacgcccgggcgcactca

SEQ ID NO: 1089 - DNA, 56 bp, Artificial Sequence
  Description of Artificial Sequence: Synthetic oligonucleotide
ggactgaggccgggcgaccaaaggtcgcccgacgcccgggcggcctcagtcctgca

SEQ ID NO: 1090 - DNA, 54 bp, Artificial Sequence
  Description of Artificial Sequence: Synthetic oligonucleotide
ctaggactgaggccgcccgggcgtcgggcgacctttggtcgcccggcctcagtc

SEQ ID NO: 1091 - DNA, 48 bp, Artificial Sequence
  Description of Artificial Sequence: Synthetic oligonucleotide
ggactgaggccgggcgaccaaaggtcgcccgacggcctcagtcctgca

SEQ ID NO: 1092 - DNA, 46 bp, Artificial Sequence
  Description of Artificial Sequence: Synthetic oligonucleotide
ctaggactgaggccgtcgggcgacctttggtcgcccggcctcagtc

SEQ ID NO: 1093 - DNA, 67 bp, Artificial Sequence
  Description of Artificial Sequence: Synthetic oligonucleotide
ggactgaggcccgggcgaccaaaggtcgcccgacgcccgggctttgcccgggcgcctcagtcctgca

SEQ ID NO: 1094 - DNA, 47 bp, Artificial Sequence
  Description of Artificial Sequence: Synthetic oligonucleotide
atacctaggcacgcgtgttactagttattaatagtaatcaattacgg

SEQ ID NO: 1095 - DNA, 29 bp, Artificial Sequence
  Description of Artificial Sequence: Synthetic oligonucleotide
atacctaggggccgcacgcgtgttactag

SEQ ID NO: 1096 - DNA, 42 bp, Artificial Sequence
  Description of Artificial Sequence: Synthetic oligonucleotide
atacactcagtgcctgcaggcacgtggtccggagatccagac

SEQ ID NO: 1097 - DNA, 3754 bp, Artificial Sequence
  Description of Artificial Sequence: Synthetic polynucleotide
cctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagg

SEQ ID NO: 1098 - DNA, 8418 bp, Artificial Sequence
  Description of Artificial Sequence: Synthetic polynucleotide
ggcagctgcgcgctcgctcgctcacctaggccgcccgggcaaagcccgggcgtcgggcgacctttggtcgcccggcctaggtgagcgagcgagcgcgcagagagggagtggccaactccatcactaggggttccttgtagttaatgattaacccgccatgctacttatcgcggccgctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagccagtttaaacgccgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaattaattaagagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctctagagcatggctacgtagataagtagcatggcgggttaatcattaactacacctgcagcaggaacccctagtgatggagttggccactccctctctgcgcgctcgctcgctccctgcaggactgaggccgggcgaccaaaggtcgcccgacgcccgggcggcctcagtcctgcagggagcgagcgagcgcgcagctgcctgcacgggcgcgccggtaccgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattgggaccaatacgcccgcgtttcttccttttccccaccccaacccccaagttcgggtgaaggcccagggctcgcagccaacgtcggggcggcaagccctgccatagccactacgggtacgtaggccaaccactagaactatagctagagtcctgggcgaacaaacgatgctcgccttccagaaaaccgaggatgcgaaccacttcatccggggtcagcaccaccggcaagcgccgcgacggccgaggtctaccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccatttattactcaaccgtaaccgattttgccaggttacgcggctggtctgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcaaataagcgttgatattcagtcaattacaaacattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcctagtgagctccctgca

SEQ ID NO: 1099 - DNA, 225 bp, Artificial Sequence; identical to SEQ ID NO: 9
  Description of Artificial Sequence: Synthetic polynucleotide

SEQ ID NO: 1100 - DNA, 1177 bp, Artificial Sequence; identical to SEQ ID NO: 74
  Description of Artificial Sequence: Synthetic polynucleotide

SEQ ID NO: 1101 - DNA, 1326 bp, Artificial Sequence
  Description of Artificial Sequence: Synthetic polynucleotide
ctgcagggcccactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaa

SEQ ID NO: 1102 - DNA, 573 bp, Mus musculus
gtaagagttttatgttttttcatctctgcttgtatttttctagtaatggaagcctggtattttaaaatagttaaattttcctttagtgctgatttctagattattattactgttgttgttgttattattgtcattatttgcatctgagaacccttaggtggttatattattgatatatttttggtatctttgatgacaataatgggggattttgaaagcttagctttaaatttcttttaattaaaaaaaaatgctaggcagaatgactcaaattacgttggatacagttgaatttattacggtctcatagggcctgcctgctcgaccatgctatactaaaaattaaaagtgtgtgttactaattttataaatggagtttccatttatatttacctttatttcttatttaccattgtcttagtagatatttacaaacatgacagaaacactaaatcttgagtttgaatgcacagatataaacacttaacgggttttaaaaataataatgttggtgaaaaaatataactttgagtgtagcagagaggaaccattgccaccttcagattttcctgt

SEQ ID NO: 1103 - DNA, 1993 bp, Mus musculus
acgatcgggaactggcatcttcagggagtagcttaggtcagtgaagagaagaacaaaaagcagcatattacagttagttgtcttcatcaatctttaaatatgttgtgtggtttttctctccctgtttccacagacaagagtgagatcgcccatcggtataatgatttgggagaacaacatttcaaaggcctgtaagttataatgctgaaagcccacttaatatttctggtagtattagttaaagttttaaaacacctttttccaccttgagtgtgagaattgtagagcagtgctgtccagtagaaatgtgtgcattgacagaaagactgtggatctgtgctgagcaatgtggcagccagagatcacaaggctatcaagcactttgcacatggcaagtgtaactgagaagcacacattcaaataatagttaattttaattgaatgtatctagccatgtgtggctagtagctcctttcctggagagagaatctggagcccacatctaacttgttaagtctggaatcttattttttatttctggaaaggtctatgaactatagttttgggggcagctcacttactaacttttaatgcaataagatctcatggtatcttgagaacattattttgtctctttgtagtactgaaaccttatacatgtgaagtaaggggtctatacttaagtcacatctccaaccttagtaatgttttaatgtagtaaaaaaatgagtaattaatttatttttagaaggtcaatagtatcatgtattccaaataacagaggtatatggttagaaaagaaacaattcaaaggacttatataatatctagccttgacaatgaataaatttagagagtagtttgcctgtttgcctcatgttcataaatctattgacacatatgtgcatctgcacttcagcatggtagaagtccatattcctttgcttggaaaggcaggtgttcccattacgcctcagagaatagctgacgggaagaggctttctagatagttgtatgaaagatatacaaaatctcgcaggtatacacaggcatgatttgctggttgggagagccacttgcctcatactgaggtttttgtgtctgcttttcagagtcctgattgccttttcccagtatctccagaaatgctcatacgatgagcatgccaaattagtgcaggaagtaacagactttgcaaagacgtgtgttgccgatgagtctgccgccaactgtgacaaatcccttgtgagtaccttctgattttgtggatctactttcctgctttctggaactctgtttcaaagccaatcatgactccatcacttaaggccccgggaacactgtggcagagggcagcagagagattgataaagccagggtgatgggaattttctgtgggactccatttcatagtaattgcagaagctacaatacactcaaaaagtctcaccacatgactgcccaaatgggagcttgacagtgacagtgacagtagatatgccaaagtggatgagggaaagaccacaagagctaaaccctgtaaaaagaactgtaggcaactaaggaatgcagagagaagaagttgccttggaagagcataccaactgcctctccaataccaatggtcatccctaaaacatacgtatgaataacatgcagactaagcaggctacatttaggaatatacatgtatttacataaatgtatatgcatgtaacaacaatgaatgaaaactgaggtcatggatctgaaagagagcaagggggcttacatgagagggtttggagggaggggttggagggagggaggtattattctttagttttacagggaacgtagtaaaaacataggcttctcccaaaggagcagagcccatgaggagctgtgcaaggttccccagcttgattttacctgctcctcaaattcccttgatttgtttttattataatgactttactcctagcttttagtgtcagatagaaaacatggaagg

SEQ ID NO: 1104 - DNA, 1350 bp, Homo sapiens
taggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttatttt

SEQ ID NO: 1105 - DNA, 1223 bp, Homo sapiens
tgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactg

SEQ ID NO: 1106 - DNA, 215 bp, Artificial Sequence
  Description of Artificial Sequence: Synthetic polynucleotide
gaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagttctgtatgagaccac

SEQ ID NO: 1107 - DNA, 141 bp, Artificial Sequence
  Description of Artificial Sequence: Synthetic polynucleotide
cctgcaggcagctgcgcgctcgctcgctcacctaggccgcccgggcaaagcccgggcgtcgggcgacctttggtcgcccggcctaggtgagcgagcgagcgcgcagagagggagtggccaactccatcactaggggttcct

SEQ ID NO: 1108 - DNA, 19 bp, Artificial Sequence
  Description of Artificial Sequence: Synthetic oligonucleotide
gcgcgctcgctcgctcacc

SEQ ID NO: 1109 - DNA, 22 bp, Artificial Sequence
  Description of Artificial Sequence: Synthetic oligonucleotide
ctaggtgagcgagcgagcgcgc

SEQ ID NO: 1110 - DNA, 75 bp, Artificial Sequence
  Description of Artificial Sequence: Synthetic oligonucleotide
cctgcaggactgaggccgcccgggcaaagcccgggcgtcgggcgacctttggtcgcccggcctcagtcctgcagg

SEQ ID NO: 1111 - DNA, 130 bp, Artificial Sequence
  Description of Artificial Sequence: Synthetic polynucleotide
aggaacccctagtgatggagttggccactccctctctgcgcgctcgctcgctcactgagtgcgggcgaccaaaggtcgcccgacgcccgggcgcactcagtgagcgagcgagcgcgcagctgcctgcagg

SEQ ID NO: 1112 - DNA, 142 bp, Artificial Sequence
  Description of Artificial Sequence: Synthetic polynucleotide
cctgcaggcagctgcgcgctcgctcgctccctaggactgaggccgcccgggcgtcgggcgacctttggtcgcccggcctcagtcctagggagcgagcgagcgcgcagagagggagtggccaactccatcactaggggttcct

SEQ ID NO: 1113 - DNA, 80 bp, Artificial Sequence
  Description of Artificial Sequence: Synthetic oligonucleotide
gcgcgctcgctcgctcactgagtgcgggcgaccaaaggtcgcccgacgcccgggcgcactcagtgagcgagcgagcgcgc

SEQ ID NO: 1114 - DNA, 21 bp, Artificial Sequence
  Description of Artificial Sequence: Synthetic oligonucleotide
gcgcgctcgctcgctcactga

SEQ ID NO: 1115 - DNA, 18 bp, Artificial Sequence
  Description of Artificial Sequence: Synthetic oligonucleotide
gtgagcgagcgagcgcgc

SEQ ID NO: 1116 - DNA, 89 bp, Artificial Sequence; identical to SEQ ID NO: 117
  Description of Artificial Sequence: Synthetic oligonucleotide

SEQ ID NO: 1117 - DNA, 89 bp, Artificial Sequence; identical to SEQ ID NO: 118
  Description of Artificial Sequence: Synthetic oligonucleotide

SEQ ID NO: 1118 - DNA, 87 bp, Artificial Sequence; identical to SEQ ID NO: 119
  Description of Artificial Sequence: Synthetic oligonucleotide

SEQ ID NO: 1119 - DNA, 87 bp, Artificial Sequence; identical to SEQ ID NO: 120
  Description of Artificial Sequence: Synthetic oligonucleotide

SEQ ID NO: 1120 - DNA, 85 bp, Artificial Sequence; identical to SEQ ID NO: 121
  Description of Artificial Sequence: Synthetic oligonucleotide

SEQ ID NO: 1121 - DNA, 85 bp, Artificial Sequence; identical to SEQ ID NO: 122
  Description of Artificial Sequence: Synthetic oligonucleotide

SEQ ID NO: 1122 - DNA, 89 bp, Artificial Sequence; identical to SEQ ID NO: 123
  Description of Artificial Sequence: Synthetic oligonucleotide

SEQ ID NO: 1123 - DNA, 89 bp, Artificial Sequence; identical to SEQ ID NO: 124
  Description of Artificial Sequence: Synthetic oligonucleotide

SEQ ID NO: 1124 - DNA, 87 bp, Artificial Sequence; identical to SEQ ID NO: 125
  Description of Artificial Sequence: Synthetic oligonucleotide

SEQ ID NO: 1125 - DNA, 87 bp, Artificial Sequence; identical to SEQ ID NO: 126
  Description of Artificial Sequence: Synthetic oligonucleotide

SEQ ID NO: 1126 - DNA, 85 bp, Artificial Sequence; identical to SEQ ID NO: 127
  Description of Artificial Sequence: Synthetic oligonucleotide

SEQ ID NO: 1127 - DNA, 85 bp, Artificial Sequence; identical to SEQ ID NO: 128
  Description of Artificial Sequence: Synthetic oligonucleotide

SEQ ID NO: 1128 - DNA, 83 bp, Artificial Sequence; identical to SEQ ID NO: 129
  Description of Artificial Sequence: Synthetic oligonucleotide

SEQ ID NO: 1129 - DNA, 83 bp, Artificial Sequence; identical to SEQ ID NO: 130
  Description of Artificial Sequence: Synthetic oligonucleotide

SEQ ID NO: 1130 - DNA, 81 bp, Artificial Sequence; identical to SEQ ID NO: 131
  Description of Artificial Sequence: Synthetic oligonucleotide

SEQ ID NO: 1131 - DNA, 81 bp, Artificial Sequence; identical to SEQ ID NO: 132
  Description of Artificial Sequence: Synthetic oligonucleotide

SEQ ID NO: 1132 - DNA, 79 bp, Artificial Sequence; identical to SEQ ID NO: 133
  Description of Artificial Sequence: Synthetic oligonucleotide

SEQ ID NO: 1133 - DNA, 79 bp, Artificial Sequence; identical to SEQ ID NO: 134
  Description of Artificial Sequence: Synthetic oligonucleotide

SEQ ID NO: 1134 - DNA, 81 bp, Artificial Sequence
  Description of Artificial Sequence: Synthetic oligonucleotide
ctgcgcgctcgctcgctcactgaggccgaaacgtcgggcgacctttggtcgcccggcctcagtgagcgagcgagcgcgcag

SEQ ID NO: 1135 - DNA, 81 bp, Artificial Sequence
  Description of Artificial Sequence: Synthetic oligonucleotide
ctgcgcgctcgctcgctcactgaggccgggcgaccaaaggtcgcccgacgtttcggcctcagtgagcgagcgagcgcgcag

SEQ ID NO: 1136 - DNA, 72 bp, Artificial Sequence; identical to SEQ ID NO: 104
  Description of Artificial Sequence: Synthetic oligonucleotide

SEQ ID NO: 1137 - DNA, 80 bp, Artificial Sequence; identical to SEQ ID NO: 17
  Description of Artificial Sequence: Synthetic oligonucleotide

SEQ ID NO: 1138 - DNA, 79 bp, Artificial Sequence; identical to SEQ ID NO: 115
  Description of Artificial Sequence: Synthetic oligonucleotide

SEQ ID NO: 1139 - DNA, 48 bp, Artificial Sequence
  Description of Artificial Sequence: Synthetic oligonucleotide
ggagtcaaagttctgtttgccctgatctgcatcgctgtggccgaggcc

SEQ ID NO: 1140 - DNA, 99 bp, Artificial Sequence
  Description of Artificial Sequence: Synthetic oligonucleotide
attcataccaacttgaagaaaaagttcagcctcttcatcctggtctttctcctgttcgcagtcatctgtgtttggaagaaagggagcgactatgaggcc

SEQ ID NO: 1141 - DNA, 588 bp, Artificial Sequence
  Description of Artificial Sequence: Synthetic polynucleotide
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 1142 - DNA, 31 bp, Artificial Sequence
  Description of Artificial Sequence: Synthetic oligonucleotide
gcccgggcgtcgggcgacctttggtcgcccg

SEQ ID NO: 1143 - DNA, 31 bp, Artificial Sequence; identical to SEQ ID NO: 1142
  Description of Artificial Sequence: Synthetic oligonucleotide

SEQ ID NO: 1144 - DNA, 2218 bp, Homo sapiens
cagggcagccttcagtctgattcaggagaacgaggtcctcttcaccatgtgcttcatccccctggtctgctggatcgtgtgcactggactgaaacagcagatggagagtggcaagagccttgcccagacatccaagacctccaccgcggtgtacgtcttcttcctttccagtttgctgcagccccggggagggagccaggagcacggcctctgcgcccacctctgggggctctgctctttggctgcagatggaatctggaaccagaaaatcctgtttgaagagtccgacctcaggaatcatggactgcagaaggcggatgtgtctgctttcctgaggatgaacctgttccaaaaggaagtggactgcgagaagttctacagcttcatccacatgactttccaggagttctttgccgccatgtactacctgctggaagaggaaaaggaaggaaggacgaacgttccagggagtcgtttgaagcttcccagccgagacgtgacagtccttctggaaaactatggcaaattcgaaaaggggtatttgatttttgttgtacgtttcctctttggcctggtaaaccaggagaggacctcctacttggagaagaaattaagttgcatgatctctcagcaaatcaggctggagctgctgaaatggattgaagtgaaagccaaagctaaaaagctgcatgatcagcccagccagctggaattgttctactgtttgtacgagatgcaggaggaggacttcgtgcaaagggccatggactatttccccaagattgagatcaatctctccaccagaatggaccacatggtttcctccttttgcattgagaactgtcatcgggtggagtcactgtccctggggtttctccataacatgcccaaggaggaagaggaggaggaaaaggaaggccgacaccttgatatggtgcagtgtgtcctcccaagctcctctcatgctgcctgttctcatgggttggggcgctgtggcctctcccatgagtgctgcttcgacatctccttggtcctcagcagcaaccagaagctggtggagctggacctgagtgacaacgccctcggtgacttcggaatcagacttctgtgtgtgggactgaagcacctgttgtgcaatctgaagaagctctggttggtgaattctgccttacgtcagtctgttgttcagctttgtcctcggtactcagcactaatcagaatctcacgcacctttactgcgaggcaacactctcggagacaagggatcaaactactctgtgagggactcttgcaccccgactgcaagcttcaggtgttggaattagacaactgcaacctcacgtcacactgctgctgggatctttccacacttctgacctccagccagagcctgcgaaagctgagcctgggcaacaatgacctgggcgacctgggggtcatgatgttctgtgaagtgctgaaacagcagagctgcctcctgcagaacctggggttgtctgaaatgtatttcaattatgagacaaaaagtgcgttagaaacacttcaagaagaaaagcctgagctgaccgtcgtctttgagccttcttggtaggagtggaaacggggctgccagacgccagtgttctccggtccctccagctgggggccctcaggtggagagagctgcgatccatccaggccaagaccacagctctgtgatccttccggtggagtgtcggagaagagagcttgccgacgatgccttcctgtgcagagcttgggcatctcctttacgccagggtgaggaagacaccaggacaatgacagcatcgggtgttgttgtcatcacagcgcctcagttagaggatgttcctctggtgacctcatgtaattagctcattcaataaagcactttctttatttttctcttctctgtctaaccttctttttcctatctttttttcttctttgttctgtttacttttgctcatatcatcattcccgctatctttctattaactgaccataacacagaactagttgactatatattatgttgaaattttatggcagctatttatttatttaaattttttgtaatagttttgttttctaataagaaaaatccatgctttttgtagctggttgaaaattcaggaatatgtaaaactttttggtatttaattaaattgattccttttcttaattttaaaaaaaaaaaaaaaaaaaaaaaaaaaaa

SEQ ID NO: 1145 - DNA, 58 bp, Artificial Sequence
  Description of Artificial Sequence: Synthetic oligonucleotide
ccggccacagtgtaacctgcagaaactcgagtttctgcaggttacactgtggtttttg

SEQ ID NO: 1146 - DNA, 16 bp, Artificial Sequence
  Description of Artificial Sequence: Synthetic oligonucleotide
gcgcgctcgctcgctc

SEQ ID NO: 1147 - DNA, 518 bp, Artificial Sequence
  Description of Artificial Sequence: Synthetic polynucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatgg

SEQ ID NO: 1148 - DNA, 777 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtaccg

SEQ ID NO: 1149 - DNA, 427 bp, Artificial Sequence
  Description of Artificial Sequence: Synthetic polynucleotide
gagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactgacatgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacatacataaggtcaataggggtgacta

SEQ ID NO: 1150 - DNA, 83 bp, Artificial Sequence
  Description of Artificial Sequence: Synthetic oligonucleotide
cgggggaggctgctggtgaatattaaccaaggtcaccccagttatcggaggagcaaacaggggctaagtccacacgcgtggta

SEQ ID NO: 1151 - DNA, 777 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctagaggtactg

SEQ ID NO: 1152 - DNA, 66 bp, Artificial Sequence
  Description of Artificial Sequence: Synthetic oligonucleotide
gtttgctgcttgcaatgtttgcccattttagggtggacacaggacgctgtggtttctgagccaggg

SEQ ID NO: 1153 - DNA, 212 bp, Artificial Sequence
  Description of Artificial Sequence: Synthetic polynucleotide
ggaggggtggagtcgtgacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggg

SEQ ID NO: 1154 - DNA, 330 bp, Artificial Sequence
  Description of Artificial Sequence: Synthetic polynucleotide
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggg

SEQ ID NO: 1155 - DNA, 194 bp, Artificial Sequence
  Description of Artificial Sequence: Synthetic polynucleotide
ccctaaaatgggcaaacattgcaagcagcaaacagcaaacacacagccctccctgcctgctgaccttggagctggggcagaggtcagagacctctctgggcccatgccacctccaacatccactcgaccccttggaatttttcggtggagaggagcagaggttgtcctggcgtggtttaggtagtgtgagaggg

SEQ ID NO: 1156 - DNA, 240 bp, Artificial Sequence
  Description of Artificial Sequence: Synthetic polynucleotide
gggcctgaaataacctctgaaagaggaacttggttaggtaccttctgaggctgaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccacta

SEQ ID NO: 1157 - DNA, 73 bp, Artificial Sequence
  Description of Artificial Sequence: Synthetic oligonucleotide
cgggggaggctgctggtgaatattaaccaaggtcaccccagttatcggaggagcaaacaggggctaagtccac

SEQ ID NO: 1158 - DNA, 100 bp, Artificial Sequence
  Description of Artificial Sequence: Synthetic polynucleotide
aggttaatttttaaaaagcagtcaaaagtccaagtggcccttggcagcatttactctctctgtttgctctggttaataatctcaggagcacaaacattcc

SEQ ID NO: 1159 - DNA, 296 bp, Artificial Sequence
  Description of Artificial Sequence: Synthetic polynucleotide
gttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctatta

SEQ ID NO: 1160 - DNA, 235 bp, Artificial Sequence
  Description of Artificial Sequence: Synthetic polynucleotide
ggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtccc

SEQ ID NO: 1161 - DNA, 1127 bp, Artificial Sequence
  Description of Artificial Sequence: Synthetic polynucleotide
ggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttttagccttcttctttttcctacagctcctgggcaacgtgctggttattgtgctgtctcatcatttgtcgacagaattcctcgaagatccgaaggggttcaagcttggcattccggtactgttggtaaagcca

SEQ ID NO: 1162 - DNA, 93 bp, Artificial Sequence
  Description of Artificial Sequence: Synthetic oligonucleotide
ctctaaggtaaatataaaatttttaagtgtataatgtgttaaactactgattctaattgtttctctcttttagattccaacctttggaactga

SEQ ID NO: 1163 - DNA, 54 bp, Artificial Sequence
  Description of Artificial Sequence: Synthetic oligonucleotide
gccctgtctcctcagcttcaggcaccaccactgacctgggacagtgaatccgga

SEQ ID NO: 1164 - DNA, 173 bp, Artificial Sequence
  Description of Artificial Sequence: Synthetic polynucleotide
ctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacaggttggtgtacagtagcttcc

SEQ ID NO: 1165 - DNA, 91 bp, Artificial Sequence
  Description of Artificial Sequence: Synthetic oligonucleotide
aagaggtaagggtttaagggatggttggttggtggggtattaatgtttaattacctggagcacctgcctgaaatcactttttttcaggttg

SEQ ID NO: 1166 - DNA, 54 bp, Artificial Sequence
  Description of Artificial Sequence: Synthetic oligonucleotide
gccctgtctcctcagcttcaggcaccaccactgacctgggacagtgaataatta

SEQ ID NO: 1167 - DNA, 147 bp, Artificial Sequence
  Description of Artificial Sequence: Synthetic polynucleotide
gccctgtctcctcagcttcaggcaccaccactgacctgggacagtgaatccggactctaaggtaaatataaaatttttaagtgtataatgtgttaaactactgattctaattgtttctctcttttagattccaacctttggaactga

SEQ ID NO: 1168 - DNA, 147 bp, Artificial Sequence
  Description of Artificial Sequence: Synthetic polynucleotide
gccctgtctcctcagcttcaggcaccaccactgacctgggacagtgaataattactctaaggtaaatataaaatttttaagtgtataatgtgttaaactactgattctaattgtttctctcttttagattccaacctttggaactga

SEQ ID NO: 1169 - DNA, 48 bp, Artificial Sequence
  Description of Artificial Sequence: Synthetic oligonucleotide
tcctcagcttcaggcaccaccactgacctgggacagtgaatcgccacc

SEQ ID NO: 1170 - DNA, 128 bp, Artificial Sequence
  Description of Artificial Sequence: Synthetic polynucleotide
gctagcaggtaagtgccgtgtgtggttcccgcgggcctggcctctttacgggttatggcccttgcgtgccttgaattactgacactgacatccactttttctttttctccacaggtttaaacgccacc

SEQ ID NO: 1171 - DNA, 98 bp, Artificial Sequence
  Description of Artificial Sequence: Synthetic oligonucleotide
aagaggtaagggtttaagttatcgttagttcgtgcaccattaatgtttaattacctggagcacctgcctgaaatcatttttttttcaggttggctagt

SEQ ID NO: 1172 - DNA, 172 bp, Artificial Sequence
  Description of Artificial Sequence: Synthetic polynucleotide
gcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtca

SEQ ID NO: 1173 - DNA, 160 bp, Artificial Sequence
  Description of Artificial Sequence: Synthetic polynucleotide
gccctgtctcctcagcttcaggcaccaccactgacctgggacagtgaatccggactctaaggtaaatataaaatttttaagtgtataatgtgttaaactactgattctaattgtttctctcttttagattccaacctttggaactgaattctagaccacc

SEQ ID NO: 1174 - DNA, 29 bp, Artificial Sequence
  Description of Artificial Sequence: Synthetic oligonucleotide
accactttcacaatctgctagcaaaggtt

SEQ ID NO: 1175 - DNA, 133 bp, Artificial Sequence
  Description of Artificial Sequence: Synthetic polynucleotide
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 1176 - DNA, 341 bp, Artificial Sequence
  Description of Artificial Sequence: Synthetic polynucleotide
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagcctcccccgttgcccctctggatccactgcttaaatacggacgaggacagggccctgtctcctcagcttcaggcaccaccactgacctgggacagtgaatcgaca

SEQ ID NO: 1177 - DNA, 316 bp, Artificial Sequence
  Description of Artificial Sequence: Synthetic polynucleotide
tctagagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggccctgcag

SEQ ID NO: 1178 - DNA, 76 bp, Artificial Sequence
  Description of Artificial Sequence: Synthetic oligonucleotide
caaagtccaggcccctctgctgcagcgcccgcgcgtccagaggccctgccagacacgcgcgaggttcgaggctgag

SEQ ID NO: 1179 - DNA, 127 bp, Artificial Sequence
  Description of Artificial Sequence: Synthetic polynucleotide
agaatgatgaaaaccgaggttggaaaaggttgtgaaaccttttaactctccacagtggagtccattatttcctctggcttcctcaaattcatattcacagggtcgttggctgtgggttgcaattacc

SEQ ID NO: 1180 - DNA, 80 bp, Artificial Sequence
  Description of Artificial Sequence: Synthetic oligonucleotide
atagcagagcaatcaccaccaagcctggaataactgcaagggctctgctgacatcttcctgaggtgccaaggaaatgagg

SEQ ID NO: 1181 - DNA, 208 bp, Artificial Sequence
  Description of Artificial Sequence: Synthetic polynucleotide
gggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcg

SEQ ID NO: 1182 - DNA, 159 bp, Artificial Sequence
  Description of Artificial Sequence: Synthetic polynucleotide
aagcttctgccttctccctcctgtgagtttggtaagtcactgactgtctatgcctgggaaagggtgggcaggagatggggcagtgcaggaaaagtggcactatgaaccctgcagccctagacaattgtactaaccttcttctctttcctctcctgacag

SEQ ID NO: 1183 - DNA, 36 bp, Artificial Sequence
  Description of Artificial Sequence: Synthetic oligonucleotide
cgcgcctagcagtgtcccagccgggttcgtgtcgcc

SEQ ID NO: 1184 - DNA, 141 bp, Artificial Sequence
  Description of Artificial Sequence: Synthetic polynucleotide
acgccgcctgggtcccagtccccgtcccatcccccggcggcctaggcagcgtttccagccccgagaactttgttctttttgtcccgccccctgcgcccaaccgcctgcgccgccttccggcccgagttctggagactcaac

SEQ ID NO: 1185 - DNA, 110 bp, Artificial Sequence
  Description of Artificial Sequence: Synthetic polynucleotide
gttggatgaaaccttcctcctactgcacagcccgcccccctacagccccggtccccacgcctagaagacagcggaactaagaaaagaagaggcctgtggacagaacaatc

SEQ ID NO: 1186 - DNA, 164 bp, Artificial Sequence
  Description of Artificial Sequence: Synthetic polynucleotide
ggtggggcggggttgagtcggaaccacaatagccaggcgaagaaactacaactcccagggcgtcccggagcaggccaacgggactacgggaagcagcgggcagcggcccgcgggaggcacctcggagatctgggtgcaaaagcccagggttaggaaccgtaggc

SEQ ID NO: 1187 - DNA, 127 bp, Artificial Sequence
  Description of Artificial Sequence: Synthetic polynucleotide
ggccaccggaattaacccttcagggctgggggccgcgctatgccccgccccctccccagccccagacacggaccccgcaggagatgggtgcccccatccgcacactgtcctttggccaccggacatc

SEQ ID NO: 1188 - DNA, 341 bp, Artificial Sequence
  Description of Artificial Sequence: Synthetic polynucleotide
tgggcaggaactgggcactgtgcccagggcatgcactgcctccacgcagcaaccctcagagtcctgagctgaaccaagaaggaggagggggtcgggcctccgaggaaggcctagccgctgctgctgccaggaattccaggttggaggggcggcaacctcctgccagccttcaggccactctcctgtgcctgccagaagagacagagcttgaggagagcttgaggagagcaggaaagcctcccccgttgcccctctggattcactgcttaaatacggacgaggacagggccctgtctcctcagcttcaggcaccaccactgacctgggacagtgaatcgaca

SEQ ID NO: 1189 - DNA, 77 bp, Artificial Sequence
  Description of Artificial Sequence: Synthetic oligonucleotide
tccataaagtaggaaacactacacgattccataaagtaggaaacactacatcactccataaagtaggaaacactaca

SEQ ID NO: 1190 - DNA, 88 bp, Artificial Sequence
  Description of Artificial Sequence: Synthetic oligonucleotide
tgaaagatggatttccaaggttaattcattggaattgaaaattaacagagatctagagctgaattcctgcagccagggggatcagcct

SEQ ID NO: 1191 - DNA, 395 bp, Artificial Sequence
  Description of Artificial Sequence: Synthetic polynucleotide
taaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagc

SEQ ID NO: 1192 - DNA, 800 bp, Artificial Sequence
  Description of Artificial Sequence: Synthetic polynucleotide
agtcaatatgttcaccccaaaaaagctgtttgttaacttgccaacctcattctaaaatgtatatagaagcccaaaagacaataacaaaaatattcttgtagaacaaaatgggaaagaatgttccactaaatatcaagatttagagcaaagcatgagatgtgtggggatagacagtgaggctgataaaatagagtagagctcagaaacagacccattgatatatgtaagtgacctatgaaaaaaatatggcattttacaatgggaaaatgatggtctttttcttttttagaaaaacagggaaatatatttatatgtaaaaaataaaagggaacccatatgtcataccatacacacaaaaaaattccagtgaattataagtctaaatggagaaggcaaaactttaaatcttttagaaaataatatagaagcatgccatcaagacttcagtgtagagaaaaatttcttatgactcaaagtcctaaccacaaagaaaagattgttaattagattgcatgaatattaagacttatttttaaaattaaaaaaccattaagaaaagtcaggccatagaatgacagaaaatatttgcaacaccccagtaaagagaattgtaatatgcagattataaaaagaagtcttacaaatcagtaaaaaataaaactagacaaaaatttgaacagatgaaagagaaactctaaataatcattacacatgagaaactcaatctcagaaatcagagaactatcattgcatatacactaaattagagaaatattaaaaggctaagtaacatctgtggc

SEQ ID NO: 1193 - DNA, 407 bp, Artificial Sequence
  Description of Artificial Sequence: Synthetic polynucleotide
aattatctctaaggcatgtgaactggctgtcttggttttcatctgtacttcatctgctacctctgtgacctgaaacatatttataattccattaagctgtgcatatgatagatttatcatatgtattttccttaaaggatttttgtaagaactaattgaattgatacctgtaaagtctttatcacactacccaataaataataaatctctttgttcagctctctgtttctataaatatgtaccagttttattgtttttagtggtagtgattttattctctttctatatatatacacacacatgtgtgcattcataaatatatacaatttttatgaataaaaaattattagcaatcaatattgaaaaccactgatttttgtttatgtgagcaaacagcagattaaaag

SEQ ID NO: 1194 - DNA, 186 bp, Artificial Sequence
  Description of Artificial Sequence: Synthetic polynucleotide
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatct

SEQ ID NO: 1195 - DNA, 395 bp, Artificial Sequence
  Description of Artificial Sequence: Synthetic polynucleotide
taaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagccagc

SEQ ID NO: 1196 - DNA, 580 bp, Artificial Sequence
  Description of Artificial Sequence: Synthetic polynucleotide
gagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttatagcctctgtatctagctattgcttcccgtacggctttcgttttctcctccttgtataaatcctggttgctgtctcttttagaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgt

SEQ ID NO: 1197 - DNA, 64 bp, Artificial Sequence
  Description of Artificial Sequence: Synthetic oligonucleotide
cctcgccccggacctgccctcccgccaggtgcacccacctgcaataaatgcagcgaagccggga

SEQ ID NO: 1198 - DNA, 247 bp, Artificial Sequence
  Description of Artificial Sequence: Synthetic polynucleotide
gataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtgg

SEQ ID NO: 1199 - DNA, 144 bp, Artificial Sequence
  Description of Artificial Sequence: Synthetic polynucleotide
aaatacatcattgcaatgaaaataaatgttttttattaggcagaatccagatgctcaaggcccttcataatatcccccagtttagtagttggacttagggaacaaaggaacctttaatagaaattggacagcaagaaagcgagc

SEQ ID NO: 1200 - DNA, 62 bp, Artificial Sequence
  Description of Artificial Sequence: Synthetic oligonucleotide
gagcatcttaccgccatttattcccatatttgttctgtttttcttgatttgggtatacattt

SEQ ID NO: 1201 - DNA, 49 bp, Artificial Sequence
  Description of Artificial Sequence: Synthetic oligonucleotide
aataaaagatctttattttcattagatctgtgtgttggttttttgtgtg

SEQ ID NO: 1202 - DNA, 54 bp, Artificial Sequence
  Description of Artificial Sequence: Synthetic oligonucleotide
gcggccgcaataaaagatcagagctctagagatctgtgtgttggttttttgtgt

SEQ ID NO: 1203 - DNA, 74 bp, Artificial Sequence
  Description of Artificial Sequence: Synthetic oligonucleotide
ggatccaataaaatatctttattttcattacatctgtgtgttggttttttgtgtgttttcctgtaacgatcggg

SEQ ID NO: 1204 - DNA, 143 bp, Artificial Sequence
  Description of Artificial Sequence: Synthetic polynucleotide
ctcgatgctttatttgtgaaatttgtgatgctattgctttatttgtaaccattataagctgcaataaacaagttaacaacaacaattgcattcattttatgtttcaggttcagggggaggtgtgggaggttttttaaactagt

SEQ ID NO: 1205 - DNA, 228 bp, Artificial Sequence
  Description of Artificial Sequence: Synthetic polynucleotide
ctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatgg

SEQ ID NO: 1206 - DNA, 222 bp, Artificial Sequence
  Description of Artificial Sequence: Synthetic polynucleotide
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 1207 - DNA, 226 bp, Artificial Sequence
  Description of Artificial Sequence: Synthetic polynucleotide
ccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatgg

SEQ ID NO: 1208 - DNA, 129 bp, Artificial Sequence
  Description of Artificial Sequence: Synthetic polynucleotide
gttaacaaaaaaaaaaaaaaaaaaaaaaaaaaaaaaaaaaaaaaaaaaaaaaaaaaaaaaaaaaaaaaaatgcatcccccccccccccccccccccccccccccccaaaggctcttttcagagccacca

SEQ ID NO: 1209 - DNA, 232 bp, Artificial Sequence
  Description of Artificial Sequence: Synthetic polynucleotide
gcggccgcggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttagtcgaccatgctggggagagatct

SEQ ID NO: 1210 - DNA, 135 bp, Artificial Sequence
  Description of Artificial Sequence: Synthetic polynucleotide
gatccagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 1211 - DNA, 49 bp, Artificial Sequence
  Description of Artificial Sequence: Synthetic oligonucleotide
cggcaataaaaagacagaataaaacgcacgggtgttgggtcgtttgttc

SEQ ID NO: 1212 - DNA, 226 bp, Artificial Sequence
  Description of Artificial Sequence: Synthetic polynucleotide
ccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgg

SEQ ID NO: 1213 - DNA, 416 bp, Artificial Sequence
  Description of Artificial Sequence: Synthetic polynucleotide
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatg

SEQ ID NO: 1214 - DNA, 415 bp, Artificial Sequence
  Description of Artificial Sequence: Synthetic polynucleotide
atcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaagttatggattataaacattcaaaataatattttgacattatgataattctgaataaaagaacaaaaaccatg

SEQ ID NO: 1215 - DNA, 122 bp, Artificial Sequence
  Description of Artificial Sequence: Synthetic polynucleotide
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 1216 - DNA, 133 bp, Artificial Sequence
  Description of Artificial Sequence: Synthetic polynucleotide
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 1217 - DNA, 64 bp, Artificial Sequence
  Description of Artificial Sequence: Synthetic oligonucleotide
aaaaaaaaaaaaaaaaaaaaaaaaaaaaaaaaaaaaaaaaaaaaaaaaaaaaaaaaaaaaaaaa

SEQ ID NO: 1218 - protein, 4 aa, Artificial Sequence
  Description of Artificial Sequence: Synthetic peptide
YVAD

SEQ ID NO: 1219 - protein, 4 aa, Artificial Sequence
  Description of Artificial Sequence: Synthetic peptide
YVHD

SEQ ID NO: 1220 - protein, 4 aa, Artificial Sequence
  Description of Artificial Sequence: Synthetic peptide
WEHD

(19) United States
(12) Patent Application Publication
(10) Pub. No.: US 2022/0119840 A1
(43) Pub. Date: Apr. 21, 2022

(54) CLOSED-ENDED DNA (CEDNA) AND USE IN METHODS OF REDUCING GENE OR NUCLEIC ACID THERAPY RELATED IMMUNE RESPONSE

(71) Applicant: Generation Bio Co., Cambridge, MA (US)

(72) Inventors: Douglas Anthony Kerr, Cambridge, MA (US); Phillip Samayoa, Cambridge, MA (US); Robert M. Kotin, Cambridge, MA (US); Matthew G. Stanton, Cambridge, MA (US); Ozan Alkan, Cambridge, MA (US); Matthew Chiocco, Cambridge, MA (US); Raj Rajendran, Cambridge, MA (US)

(21) Appl. No.: 17/424,199

(22) PCT Filed: Jan. 24, 2020
(86) PCT No.: PCT/US2020/015026

§ 371 Date: Jul. 20, 2021

Related U.S. Application Data
(60) Provisional application No. 62/796,417, filed on Jan. 24, 2019.
(60) Provisional application No. 62/796,450, filed on Jan. 24, 2019.
(60) Provisional application No. 62/800,285, filed on Feb. 1, 2019.
(60) Provisional application No. 62/800,303, filed on Feb. 1, 2019.
(60) Provisional application No. 62/814,414, filed on Mar. 6, 2019.
(60) Provisional application No. 62/814,424, filed on Mar. 6, 2019.
(60) Provisional application No. 62/857,542, filed on Jun. 5, 2019.

(51) Int. Cl. C12N 15/86

(52) U.S. Cl. CPC C12N 15/86; A61K 48/00

(57) ABSTRACT

Provided herein are methods and constructs related to minimizing immune responses using inhibitors of the immune response, in particular the innate immune response, when administering a desired transgene in a cell achieved by delivery of the transgene with repeated doses of a ceDNA vector.

### RELATED APPLICATIONS

This application claims priority to U.S. Provisional Application No. 62/796,417, filed on Jan. 24, 2019, U.S. Provisional Application No. 62/800,303, filed on Feb. 1, 2019, U.S. Provisional Application No. 62/796,450, filed on Jan. 24, 2019, U.S. Provisional Application No. 62/800,285, filed on Feb. 1, 2019, U.S. Provisional Application No. 62/814,414, filed on Mar. 6, 2019, U.S. Provisional Application No. 62/814,424, filed on Mar. 6, 2019, and U.S. Provisional Application No. 62/857,542, filed on Jun. 5, 2019, the contents of each of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Jan. 24, 2020, is named 131698-03320-Sequence_Listing-FINAL.txt and is 117,124 bytes in size.

### TECHNICAL FIELD

Embodiments of the invention relate to the field of gene therapy, including the delivery of exogenous DNA sequences to a target cell, tissue, organ or organism, and modifications and methods for inhibiting immune responses (e.g., innate immune responses) to the same.

### BACKGROUND

Gene therapy aims to improve clinical outcomes for patients suffering from either genetic mutations or acquired diseases caused by an aberration in the gene expression profile. Gene therapy includes the treatment or prevention of medical conditions resulting from defective genes or abnormal regulation or expression, e.g. underexpression or overexpression, that can result in a disorder, disease, malignancy, etc. For example, a disease or disorder caused by a defective gene might be treated, prevented or ameliorated by delivery of a corrective genetic material to a patient, or might be treated, prevented or ameliorated by altering or silencing a defective gene, e.g., with a corrective genetic material to a patient resulting in the therapeutic expression of the genetic material within the patient.

The basis of gene therapy is to supply a transcription cassette with an active gene product (sometimes referred to as a transgene), e.g., that can result in a positive gain-of-function effect, a negative loss-of-function effect, or another outcome. Such outcomes can be attributed to expression of an activating antibody or fusion protein or an inhibitory (neutralizing) antibody or fusion protein. Gene therapy can also be used to treat a disease or malignancy caused by other factors. Human monogenic disorders can be treated by the delivery and expression of a normal gene to the target cells. Delivery and expression of a corrective gene in the patient's target cells can be carried out via numerous methods, including the use of engineered viruses and viral gene delivery vectors. Among the many virus-derived vectors available (e.g., recombinant retrovirus, recombinant lentivirus, recombinant adenovirus, and the like), recombinant adeno-associated virus (rAAV) is gaining popularity as a versatile vector in gene therapy.

Adeno-associated viruses (AAV) belong to the parvoviridae family and more specifically constitute the dependoparvovirus genus. Vectors derived from AAV (i.e., recombinant AAV (rAVV) or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the rep gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see ii), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Additionally, conventional AAV virions with capsids are produced by introducing a plasmid or plasmids containing the AAV genome, rep genes, and cap genes (Grimm et al., 1998). However, such encapsidated AAV virus vectors were found to inefficiently transduce certain cell and tissue types and the capsids also induce an immune response. Accordingly, use of adeno-associated virus (AAV) vectors for gene therapy is limited due to the single administration to patients (owing to the patient immune response), the limited range of transgene genetic material suitable for delivery in AAV vectors due to minimal viral packaging capacity (about 4.5 kb), and slow AAV-mediated gene expression.

Moreover, mammalian immune systems include a number of mechanisms to detect and eliminate invading pathogens and aberrant cellular activities and processes, which can be elicited in the presence of administration of a viral vector or nucleic acid to a subject. For example, pattern recognition receptors (PRRs) are a class of molecules that evolved to act as sensors for the detection of conserved pathogen-associated molecules, such as foreign nucleic acids, e.g., viral DNA and viral RNA, and to trigger the innate immune response. The Toll-like receptors (TLRs) are a group of PRRs that detect nucleic acids in the context of the endosome, and include TLR9 (detects dsDNA, preferentially unmethylated CpG repeats), TLR3 (detects dsRNA), and TLR7 (detects ssRNA). A second system of PRRs are located in the cytosol for detecting foreign nucleic acid, specifically double-stranded RNA, within infected cells.¹ These PRRs, termed “RIG-I-like receptors” or RLRs, include RIG-I and MDAS. These PRRs are helicases that detect structural features of RNA, such as 5′ triphosphates and diphosphates, RNA replication intermediates, and/or transcription products, and initiate activation of the type I interferon response.^{1, 2} A third class of PRRs are triggered by cytosolic DNA, with the main intracellular DNA sensor being cGAS (cyclic GMP-AMP synthase), which binds to DNA and activates the ER-bound stimulator of interferon genes (STING), resulting in activation of the type I interferon response and, in some cases, activation of ^{1,4,5} other proposed cytosolic DNA sensors including Absent in Melanoma (AIM2), IFN-γ-inducible protein 16 (IFI16), Interferon-Inducible Protein X (IFIX), LRRFIP1, DHX9, DHX36, DDX41, Ku70, DNA-PKcs, MRN complex (including MRE11, Rad50 and Nbs1)^{2,7} and RNA polymerase III¹⁰. AIM2, IFI16, and IFIX are pyrin and HIN200 domain proteins (PYHIN) proteins.^{2,6} Furthermore, it has been shown that unpaired DNA nucleotides flanking short base-paired DNA stretches, as in stem-loop structures of single-stranded DNA (ssDNA) derived from human immunodeficiency virus type 1 (HIV-1), activated the type I interferon-inducing DNA sensor cGAS in a sequence-dependent manner.^{8,9} DNA structures containing unpaired guanosines flanking short (12- to 20-bp) dsDNA (Y-form DNA) were highly stimulatory and specifically enhanced the enzymatic activity of cGAS.^{8,9}

More recently, other intracellular microbial sensors have been identified, including NOD-like receptors (NLRs). Some of the NLRs also sense nonmicrobial danger signals and form large cytoplasmic protein complexes called inflammasomes which are a central regulator of innate immunity and inflammation (Martinon et al., Annu. Rev. Immunol. 2009 27: 229-65).

The inflammasome is composed of NLR or AIM2 family receptors and procaspase-1. An apoptosis-associated speck-like protein containing a caspase recruitment domain (ASC) is an adaptor protein, and links the NLR family member to procaspase-1. NLR family members assemble an inflammasome complex with ASC, which in turn recruits and activates caspase-1. Several members of the NLR family proteins participate in the formation of distinct inflammasomes, including NLR family pyrin domain-containing 3 (NLRP3; also known as cyropyrin or NALP3), NLR family CARD domain-containing 4 (NLRC4; also known as IPAF), and NLRP1. Different inflammasomes are activated by various stimuli. For example, NLRP1 becomes activated by the lethal toxin produced by Bacillus anthracis, whereas NLRC4 responds to cytosolic flagellin in cells infected with Salmonella, Legionella, and Pseudomonas spp. The NLRP3 inflammasome is activated by a large variety of stimuli, including microbial products and endogenous signals, such as urate crystal, silica, amyloid fibrils, and ATP.

The NOD-like receptor (NLR) sensor component (i.e., cryopyrin (NLRP3 or NALP3)), recognize danger signals such as Damage associated molecular pattern molecules (DAMPs) released during tissue injury or stress (e.g., extracellular ATP, urate crystal, β-amyloid, cell debris) and Pathogen-Associated Molecular Patterns (PAMPs). The inflammasome is assembled in response to these pathogen infection or “danger” signals, requiring the interaction of the pyrin domains of cryopyrin and the adaptor component ASC, which leads to the recruitment of and activation of caspase-1 (from pro-caspase-1) and subsequently to maturation and release of several proinflammatory cytokines, including interleukin-1β (IL-1β), IL-18, and IL-33).

Besides NLRs, AIM2 family members can activate inflammasomes. AIM2 is characterized by the presence of a pyrin domain and a DNA-binding HIN domain and activates caspase-1 by detecting cytosolic DNA (Fernandes-Alnemri T, et al. 2009. Nature 458:509-513). Assembly of the inflammasome requires a preceding priming signal via TLRs which is required to upregulate the expression of inflammasome receptors and the substrate pro-IL-1β, before the second signal can initiate inflammasome complex formation (Bauernfeind F G, et al. 2009.J. Immunol. 183:787-791).

Although conceptually elegant, the prospect of using nucleic-acid molecules for gene therapy for treating human diseases remains uncertain. The main cause of this uncertainty is the apparent adverse events relating to host's innate immune response to nucleic acid therapeutics and, thus, the way in which these materials modulate expression of their intended targets in the context of the immune response. The current state of the art surrounding the creation, function, behavior and optimization of nucleic acid molecules that may be adopted for clinical applications has a particular focus on: (1) antisense oligonucleotides and duplex RNAs that directly regulate translation and gene expression; (2) transcriptional gene silencing RNAs that result in long-term epigenetic modifications; (3) antisense oligonucleotides that interact with and alter gene splicing patterns; (4) creation of synthetic or viral vectors that mimic physiological functionalities of naturally occurring AAV or lentiviral genome; and (5) the in vivo delivery of therapeutic oligonucleotides. However, despite the advances made in the development of nucleic acid therapeutics that are evident in recent clinical achievements, the field of gene therapy is still severely limited by unwanted adverse events in recipients triggered by the therapeutic nucleic acids, themselves.

Accordingly, there is a need in the field for a new technology that inhibits (e.g., reduces, ameliorates, mitigates, prevents) the immune response on administration of vectors or nucleic acid to a subject that permits expression of a therapeutic protein in a cell, tissue or subject for the treatment of a wide variety of diseases.

### SUMMARY

The present disclosure provides methods and pharmaceutical compositions for inhibiting (i.e., reducing or suppressing) an immune response in a subject suffering from a genetic disorder and receiving gene or nucleic acid therapy (“nucleic acid therapeutics” or “therapeutic nucleic acid” (TNA)). Provided herein are non-viral capsid-free DNA vectors with covalently-closed ends (ceDNA vectors) and inhibitors for inhibiting an immune response (e.g., an innate immune response). According to some embodiments, the pharmaceutical compositions and formulations may include one or more inhibitors of the immune response (e.g., the innate immune response), such as rapamycin and rapamycin analogs thereof, TLR antagonists (e.g., TLR9 antagonists), cGAS antagonists and inflammasome antagonists (e.g., any one or more of: an inhibitor of the NLRP3 inflammasome pathway, or an inhibitor of the AIM2 inflammasome pathway, or an inhibitor of caspase 1, or any combination thereof).

According to some aspects, the disclosure provides compositions and methods for inhibiting (i.e., reducing or suppressing) an immune response (e.g., an innate immune response) using non-viral, capsid-free DNA vectors with covalently-closed ends (ceDNA vectors) for expressing an inhibitor of the innate immune response from a capsid-free (e.g., non-viral) DNA vector with covalently-closed ends (referred to herein as a “closed-ended DNA vector” or a “ceDNA vector”), where the ceDNA vector comprises a nucleic acid sequence or codon optimized versions thereof of an inhibitor of the immune response (e.g., the innate immune response).

According to some aspects, the disclosure provides compositions and methods for inhibiting (i.e., reducing or suppressing) an immune response (e.g., an innate immune response) using non-viral, capsid-free DNA vectors with covalently-closed ends (ceDNA vectors) for expressing rapamycin and rapamycin analogs thereof, from a capsid-free (e.g., non-viral) DNA vector with covalently-closed ends (referred to herein as a “closed-ended DNA vector” or a “ceDNA vector”), where the ceDNA vector comprises a nucleic acid sequence or codon optimized versions thereof of rapamycin and rapamycin analogs thereof. Accordingly, these ceDNA vectors can be used to produce rapamycin and rapamycin analogs thereof, for inhibiting the immune system (e.g., the innate immune system).

According to some aspects, the disclosure provides compositions and methods for inhibiting (i.e., reducing or suppressing) an immune response (e.g., an innate immune response) using non-viral, capsid-free DNA vectors with covalently-closed ends (ceDNA vectors) for expressing a TLR antagonist, from a capsid-free (e.g., non-viral) DNA vector with covalently-closed ends (referred to herein as a “closed-ended DNA vector” or a “ceDNA vector”), where the ceDNA vector comprises a nucleic acid sequence or codon optimized versions thereof of a TLR antagonist. Accordingly, these ceDNA vectors can be used to produce a TLR antagonist, for inhibiting the immune system (e.g., the innate immune system).

According to some aspects, the disclosure provides compositions and methods for inhibiting (i.e., reducing or suppressing) an immune response (e.g., an innate immune response) using non-viral, capsid-free DNA vectors with covalently-closed ends (ceDNA vectors) for expressing a cGAS antagonist, from a capsid-free (e.g., non-viral) DNA vector with covalently-closed ends (referred to herein as a “closed-ended DNA vector” or a “ceDNA vector”), where the ceDNA vector comprises a nucleic acid sequence or codon optimized versions thereof of a cGAS antagonist. Accordingly, these ceDNA vectors can be used to produce a cGAS antagonist, for inhibiting the immune system (e.g., the innate immune system).

According to some aspects, the disclosure provides compositions and methods for inhibiting (i.e., reducing or suppressing) an immune response (e.g., an innate immune response) using non-viral, capsid-free DNA vectors with covalently-closed ends (ceDNA vectors) for expressing an inhibitor of the NLRP3 inflammasome pathway, or an inhibitor of the AIM2 inflammasome pathway, or an inhibitor of caspase 1, or any combination thereof, from a capsid-free (e.g., non-viral) DNA vector with covalently-closed ends (referred to herein as a “closed-ended DNA vector” or a “ceDNA vector”), where the ceDNA vector comprises a nucleic acid sequence or codon optimized versions thereof of an inhibitor of the NLRP3 inflammasome pathway, or an inhibitor of the AIM2 inflammasome pathway, or an inhibitor of caspase 1, or any combination thereof. Accordingly, these ceDNA vectors can be used to produce an inhibitor of the NLRP3 inflammasome pathway, or an inhibitor of the AIM2 inflammasome pathway, or an inhibitor of caspase 1, or any combination thereof, for inhibiting the immune system (e.g., innate immune system).

According to some embodiments, the pharmaceutical compositions and formulations may include one or more inhibitors of the immune response (e.g., innate immune response), as described herein, in, in conjunction with various types of therapeutic nucleic acids (TNA) and carriers (e.g., lipid nanoparticle). According to some embodiments, the composition further comprises an excipient or carrier. According to some embodiments, the pharmaceutical composition comprises a lipid nanoparticle (LNP). In one embodiment, the LNP comprises a cationic lipid. According to some embodiments, the LNP comprises polyethylene glyclol (PEG). According to some embodiments, the LNP comprises a cholesterol.

The methods described herein generally include use of one or more inhibitors of the immune response (e.g., innate immune response) (e.g., rapamycin and analogs thereof, TLR antagonists, cGAS antagonists) for preventing, reducing, attenuating or even eliminating immune responses associated with administration of a transgene (e.g., a therapeutic nucleic acid (TNA)). Methods comprising administering the same are described herein.

In one embodiment, the therapeutic nucleic acid is an RNA molecule, or a derivative thereof. In one embodiment, the RNA molecule is an antisense oligonucleotide. In one embodiment, the antisense oligonucleotide is an antisense RNA. In one embodiment, the RNA is RNA interference (RNAi).

In one embodiment, the therapeutic nucleic acid is an mRNA molecule.

In one embodiment, the therapeutic nucleic acid is a DNA molecule, or a derivative thereof.

In one embodiment, the therapeutic nucleic acid is a DNA antisense oligonucleotide.

In one embodiment, the DNA antisense oligonucleotide is morpholino based nucleic acid. In one embodiment, the morpholino based nucleic acid is a phosphorodiamidate morpholino oligomer (PMO).

In one embodiment, the therapeutic nucleic acid is a closed-ended DNA (ceDNA). In one embodiment, the ceDNA comprises an expression cassette comprising a promoter sequence and a transgene. In one embodiment, the ceDNA comprises expression cassette comprising a polyadenylation sequence. In one embodiment, the ceDNA comprises at least one inverted terminal repeat (ITR) flanking either 5′ or 3′ end of the expression cassette. In one embodiment, the expression cassette is flanked by two ITRs, wherein the two ITRs comprise one 5′ ITR and one 3′ ITR. In one embodiment, the expression cassette is connected to an ITR at 3′ end (3′ ITR). In one embodiment, the expression cassette is connected to an ITR at 5′ end (5′ ITR). In one embodiment, the ceDNA further comprises a spacer sequence between a 5′ ITR and the expression cassette.

In one embodiment, the ceDNA further comprises a spacer sequence between a 3′ ITR and the expression cassette. In one embodiment, the spacer sequence is at least 5 base pair long in length. In one embodiment, the spacer sequence is 5 to 200 base pairs long in length. In one embodiment, the spacer sequence is 5 to 500 base pairs long in length.

In one embodiment, the ITR is an ITR derived from an AAV serotype. In one embodiment, the AAV is selected from the group consisting of AAV1, AAV2, AAV3, AAV4, AAV5, AAV6, AAV7, AAV8, AAV9, AAV10, AAV11 and AAV12. In one embodiment, the ITR is derived from an ITR of goose virus. In one embodiment, the ITR is derived from a B19 virus ITR. In one embodiment, the ITR is a wild-type ITR from a parvovirus. In one embodiment, the ITR is a mutant ITR. In one embodiment, the ceDNA comprises two mutant ITRs in both 5′ and 3′ ends of the expression cassette.

In one embodiment, the ceDNA has a nick or a gap.

In one embodiment, the ceDNA is synthetically produced in a cell-free environment.

In one embodiment, the ceDNA is produced in a cell. In one embodiment, the ceDNA is produced in insect cells. In one embodiment, the insect cell is Sf9. In one embodiment, the ceDNA is produced in a mammalian cell. In one embodiment, the mammalian cell is human cell line.

In one embodiment, the therapeutic nucleic acid is a closed-ended DNA comprising at least one protelomerase target sequence in its 5′ and 3′ ends of the expression cassette.

In one embodiment, the therapeutic nucleic acid is a dumbbell shaped linear duplex closed-ended DNA comprising two hairpin structures of ITRs in 5′ and 3′ ends of an expression cassette.

In one embodiment, the therapeutic nucleic acid is a DNA-based minicircle or a MIDGE.

In one embodiment, the therapeutic nucleic acid is a linear covalently closed-ended DNA vector. In one embodiment, the linear covalently closed-ended DNA vector is a ministring DNA.

In one embodiment, the therapeutic nucleic acid is a doggybone (dbDNA™) DNA.

In one embodiment, the therapeutic nucleic acid is a minigene.

In one embodiment, the therapeutic nucleic acid is a plasmid.

Accordingly, provided herein, in some aspects are methods for inhibiting or suppressing immune responses when expressing a transgene in a cell, comprising: co-administering to a cell (1) a composition comprising a non-viral capsid-free DNA vector with covalently-closed ends (ceDNA vector) and (2) an inhibitor of an immune response (e.g., an innate immune response), as described herein. The ceDNA vector comprises a heterologous nucleic acid sequence encoding a transgene operably positioned between two different AAV inverted terminal repeat sequences (ITRs), one of the ITRS comprising a functional AAV terminal resolution site and a Rep binding site, one of the ITRs comprising a deletion, insertion, or substitution relative to the other ITR, and such that the ceDNA vector when digested with a restriction enzyme having a single recognition site on the ceDNA vector has the presence of characteristic bands of linear and continuous DNA as compared to linear and non-continuous DNA controls when analyzed on a non-denaturing gel. As shown herein, in some embodiments, the inhibitor of the immune response (e.g., the innate immune response) is co-administered using a synthetic nanocarrier as described in WO 2016/073799, the contents of which are incorporated herein by reference in their entirety. In some embodiments, the ceDNA vector is also present in the nanocarrier. According to some embodiments, one or more inhibitors of the immune response (e.g., the innate immune response), are selected from rapamycin and rapamycin analogs thereof, TLR antagonists (e.g., TLR9 antagonists), cGAS antagonists and inflammasome antagonists (e.g., any one or more of: an inhibitor of the NLRP3 inflammasome pathway, or an inhibitor of the AIM2 inflammasome pathway, or an inhibitor of caspase 1, or any combination thereof). According to some embodiments, the TLR9 inhibitory oligonucleotide is present on at least one of the ITRs. According to some embodiments, the inhibitor of cGAS is encoded by the ceDNA and operably linked to a promoter, such as an inducible promoter. In other embodiments, the inhibitor of cGAS is not encoded by the ceDNA.

Further, provided herein, in one aspect is a composition comprising (i) a non-viral capsid-free DNA vector with covalently-closed ends (ceDNA vector), wherein the ceDNA vector comprises a heterologous nucleic acid sequence encoding the transgene operably positioned between two different AAV inverted terminal repeat sequences (ITRs), one of the ITRsS comprising a functional AAV terminal resolution site and a Rep binding site, one of the ITRs comprising a deletion, insertion, or substitution relative to the other ITR, wherein the ceDNA vector when digested with a restriction enzyme having a single recognition site on the ceDNA vector has the presence of characteristic bands of linear and continuous DNA as compared to linear and non-continuous DNA controls when analyzed on a non-denaturing gel, and (ii) an inhibitor of the immune response (e.g., the innate immune response). As shown herein, in some embodiments, the components of the composition are formulated in separate synthetic nanocarriers. In one embodiment, the components of the composition are formulated in the same synthetic nanocarrier. According to some embodiments, one or more inhibitors of the immune response (e.g., the innate immune response), are selected from rapamycin and rapamycin analogs thereof, TLR antagonists (e.g., TLR9 antagonists), cGAS antagonists and inflammasome antagonists (e.g., any one or more of: an inhibitor of the NLRP3 inflammasome pathway, or an inhibitor of the AIM2 inflammasome pathway, or an inhibitor of caspase 1, or any combination thereof).

The non-viral capsid free DNA vectors described herein can be produced in permissive host cells from an expression construct (e.g., a plasmid, a Bacmid, a baculovirus, or an integrated cell-line) e.g., see the Examples disclosed in International Patent Application PCT/US18/49996 filed on Sep. 7, 2018, or using synthetic production, e.g., see the Examples disclosed in International Patent Application PCT/US19/14122, filed Dec. 6, 2018, each of which are incorporated herein in their entirety by reference. In some embodiments, the ceDNA vectors useful in the methods and compositions as disclosed herein comprise a heterologous nucleic acid, e.g. a transgene positioned between two inverted terminal repeat (ITR) sequences. In some embodiments, at least one of the ITRs is modified by deletion, insertion, and/or substitution as compared to a wild-type ITR sequence (e.g. AAV ITR); and at least one of the ITRs comprises a functional terminal resolution site (TRS) and a Rep binding site.

According to another aspect, the disclosure features a method of treating a genetic disorder in a subject, the method comprising administering to the subject an effective amount of a pharmaceutical composition disclosed herein.

### BRIEF DESCRIPTION OF THE DRAWINGS

FIG. 1 is a schematic illustrating one embodiment of an upstream process for making baculo-infected insect cells (BIICs) that are useful in the production of ceDNA vector in the process described in the schematic in FIG. 2. i) Two populations of Naïve insect cells are transfected with either Rep protein plasmid or DNA vector producing plasmid; ii) viral supernatant is harvested and used to infect tow new naïve populations of insect cells to generate BIICS-1 of DNA vector construct and BIICS-2 (REP). BIICS refers to baculovirus infected insect cells. Optionally, step ii) can be repeated one or multiple times to produce the recombinant baculovirus in larger amounts.

FIG. 2 is a schematic illustrating one embodiment for production of the ceDNA vector described herein.

FIG. 3 is a schematic illustrating one embodiment for characterization of the DNA vector described herein (downstream process).

FIG. 4A to FIG. 4D are schematic diagrams illustrating exemplary plasmids and components of the plasmid that are useful in making the ceDNA vector disclosed herein. FIG. 4A shows an exemplary Rep plasmid and FIG. 4B shows an exemplary plasmid TTX vector plasmid that contains the ceDNA vector template. FIG. 4C and FIG. 4D are schematics of exemplary functional components of the DNA vector template useful in making the ceDNA vectors provided herein. The transgene, also referred to as nucleic acid of interest (e.g. reporter nucleic acid such as luciferase, or e.g. a therapeutic nucleic acid), is positioned between two different ITRs. The modified ITR can be orientated in the template either on the left hand (FIG. 4C) or right hand side (FIG. 4D). In addition, the nucleic acid of interest can be operably linked to promoter, enhancer, and termination elements. In alternative embodiments, the ITR on the left (5′ITR) or right (3′ ITR) can be any type. For exemplary purposes, the ITRs in the ceDNA constructs in FIG. 4C and FIG. 4D and in the Examples herein show a modified ITR (ΔITR) and a WT ITR (ITR) and is an example of an asymmetric ITR pair. However, encompassed herein are ceDNA vectors that contain a heterologous nucleic acid sequence (e.g., a transgene) positioned between any two inverted terminal repeat (ITR) sequences, where the ITR sequences can be an asymmetrical ITR pair or a symmetrical- or substantially symmetrical ITR pair, as these terms are defined herein. A ceDNA vector comprising a NLP as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

FIG. 5A and FIG. 5B are drawings that illustrate one embodiment for identifying the presence of the DNA vectors described herein. FIG. 5A illustrates DNA having a non-continuous structure (non-closed DNA, e.g. control cassette DNA isolated from the template TTX vector having open ends) and exemplary characteristic bands produced when cut by a restriction endonuclease having a single recognition site on the non-continuous DNA, e.g. observation of two DNA fragments of different expected sizes (e.g. 1 kb and 2 kb) under denaturing conditions. FIG. 5B illustrates DNA having a close-ended linear and continuous structure and exemplary characteristic bands produced when cut by a restriction endonuclease having a single recognition site on the linear duplex continuous DNA, e.g. observation of two DNA fragments of different sizes, (e.g. 2 kb and 4 kb) under denaturing conditions, which is 2× greater than would be expected in the event the DNA were non-continuous. Although the DNA is denatured, the complementary strands are covalently-bound and the resulting denatured products are single-stranded DNA with double the length of the corresponding non-continuous products.

FIG. 6 is an exemplary non-denaturing gel showing the presence of the highly stable DNA vectors and characteristic bands confirming the presence of highly stable close-ended DNA (ceDNA vector).

FIG. 7 is a gel and quantification standard curve for evaluating DNA material produced by processes disclosed herein.

FIG. 8 is a western blot analysis of FIX protein expressed from HEK293 cells containing various constructs and visualized using Factor IX antibody.

FIG. 9 provides a graphical depiction of the results of Example 24. The hydrodynamically administered samples show significant elevation in total flux (e.g., luciferase expression) relative to the non-hydrodynamically administered samples over the threeday study period.

FIGS. 10A and 10B provides data from the THP-1 cultured cell experiments described in the Examples assessing interferon response in cells treated with ceDNA vector and immune inhibitors. FIG. 10A shows interferon pathway activation in response to ceDNA in THP-1 cells with intact cGAS/STING and TLR9 pathways, but lack of activation in the same cells in which either pathway is impaired. Separately, inclusion of either inhibitor A151 or BX795 similarly reduce this interferon pathway activation. FIG. 10B is a similar experiment showing the dose-dependency of interferon induction inhibition with A151 and AS1411. In each grouping of bars, the 2.5 μM dose is on the left, the 1.25 μM dose is in the middle, and the 0.625 μM dose is on the right.

FIGS. 11A and 11B provides graphs of the data obtained in Example 26. FIG. 11A shows the reduction of NF-κB induction upon ceDNA administration when CpG present in the ceDNA are methylated prior to administration to the cells. FIG. 11B further shows that inclusion of the immune inhibitor A151 reduced the ceDNA-stimulated NF-κB induction to the same degree as methylation of CpG in this assay.

FIG. 12A-FIG. 12C provides the results of the experiments described in Example 26. FIG. 12A and FIG. 12B are graphs of data from each of the cytokine induction assays performed on the blood samples taken from ceDNA vector-treated mice or LNP-poly C control-treated mice, with the specific cytokine being interrogated reflected at the top of each graph. FIG. 12C provides data from the ceDNA-driven luciferase expression assay in treated mice, showing total flux in each group of mice over the duration of the study. High levels of unmethylated CpG correlated with lower total flux observed in the mice.

FIG. 13 provides the total flux data obtained from the experiments described in Example 27 in neonatal day 8 mice. Over the course of the study, ceDNA-High CpG decreased in flux over the course of the assay while ceDNA with reduced or no unmethylated CpG maintained luciferase expression. A single redose modestly increased the observed expression levels in the CpG-minimized or CpG-absent samples, but this sustained increase upon redose was not observed in the High CpG sample groups.

FIG. 14A-FIG. 14C provides results from the experiments described in Example 28. FIG. 14A and FIG. 14B are graphs of data from each of the cytokine induction assays performed on the blood samples taken from ceDNA vector-treated mice with mutant STING genetic background or polyC control-treated samples, with the specific cytokine being interrogated reflected at the top of each graph. With the exception of IL-18, significantly less induction of cytokines was observed in low and no-methylated CpG ceDNA contexts. FIG. 14C provides data from the ceDNA-driven luciferase expression assay in treated mutant STING mice, showing total flux in each group of mice over the duration of the study. The findings again showed a correlation between high levels of unmethylated CpG in the ceDNA and lower total flux observed.

FIG. 15A and FIG. 15B show the expression of the Padua FIX and FIX transgenes from highly stable DNA vectors disclosed herein. Quantataive analysis of FIX protein levels expressed from the plasmids or vectors were also assessed using the VisuLize Factor IX ELISA kit (Affinity Biologicals, #FIX-AG), following the protocols provided by the vendor.

FIGS. 16A and 16B depict the results of the ceDNA persistence and redosing study in Rag2 mice described in Example 10. FIG. 16A shows a graph of total flux over time observed in LNP-ceDNA-Luc-treated wild-type c57bl/6 mice or Rag2 mice. FIG. 16B provides a graph showing the impact of redose on expression levels of the luciferase transgene in Rag2 mice, with resulting increased stable expression observed after redose (arrow indicates time of redose administration).

FIG. 17 provides data from the ceDNA luciferase expression study in treated mice described in Example 29, showing total flux in each group of mice over the duration of the study. High levels of unmethylated CpG correlated with lower total flux observed in the mice over time, while use of a liver-specific promoter correlated with durable, stable expression of the transgene from the ceDNA vector over at least 77 days.

FIG. 18A-18H show cytokine levels of after ceDNA vector administration with pharmacologic macrophage depletion with a NLRP3 inhibitor (MCC950) or Caspase 1 inhibitor (VX765). FIG. 18A shows IFN-α levels, FIG. 18B shows IFN-γ levels, showing significant reduction of IFN-γ with the NLRP3 inhibitor MCC950 (see arrow), FIG. 18C shows IL-β levels, FIG. 18D shows IL-18 levels showing significant reduction of IFN-γ with the NLRP3 inhibitor MCC950 (see arrow), FIG. 18E shows IL-6 levels, FIG. 18F shows IP-10 levels, FIG. 18G shows MCP-1 levels, FIG. 18H shows TNFα levels.

### DETAILED DESCRIPTION

Nucleic acid transfer vectors and therapeutic agents are promising therapeutics for a variety of applications, such as gene expression and modulation thereof. Viral transfer vectors may comprise transgenes that encode proteins or nucleic acids. Examples of such include AAV vectors, microRNA (miRNA), small interfering RNA (siRNA), as well as antisense oligonucleotides that bind mutation sites in messenger RNA (such as small nuclear RNA (snRNA)). Unfortunately, the promise of these therapeutics has not yet been realized, in large part due to cellular and humoral immune responses directed against the viral transfer vector. These immune responses include antibody, B cell and T cell responses, and are often specific to viral antigens of the viral transfer vector, such as viral capsid or coat proteins or peptides thereof.

Currently, many potential patients harbor some level of pre-existing immunity against the viruses on which viral transfer vectors are based. In fact, antibodies against viral nucleic acids (both DNA and RNA) or protein are highly prevalent in the human population. In addition, even if the level of pre-existing immunity is low, for example, due to the low immunogenicity of the viral transfer vector, such low levels may still prevent successful transduction (e.g., Jeune, et al., Human Gene Therapy Methods, 24:59-67 (2013)). Thus, even low levels of pre-existing immunity may hinder the use of a specific viral transfer vector in a patient, and may require a clinician to choose a viral transfer vector based on a virus of a different serotype that may not be as efficacious, or even opt out for a different type of therapy altogether if another viral transfer vector therapy is not available.

Additionally, viral vectors, such as adeno-associated vectors, can be highly immunogenic and elicit humoral and cell-mediated immunity that can compromise efficacy, particularly with respect to re-administration. In fact, cellular and humoral immune responses against a viral transfer vector can develop after a single administration of the viral transfer vector. After viral transfer vector administration, neutralizing antibody titers can increase and remain high for several years, and can reduce the effectiveness of re-administration of the viral transfer vector. Indeed, repeated administration of a viral transfer vector generally results in enhanced, undesired immune responses. In addition, viral transfer vector-specific CD8+ T cells may arise and eliminate transduced cells expressing a desired transgene product, for example, on re-exposure to a viral antigen like viral nucleic acid or capsid protein. For example, it has been shown that AAV nucleic acids or capsid antigens can trigger immune-mediated destruction of hepatocytes transduced with an AAV viral transfer vector. For many therapeutic applications, it is thought that multiple rounds of administration of viral transfer vectors are needed for long-term benefits. The ability to do so, however, would be severely limited, particularly if re-administration is needed, without the methods and compositions provided herein.

Methods and compositions are provided that offer solutions to the aforementioned obstacles to effective use of variety of nucleic acid therapeutics, including viral or non-viral (synthetic) transfer vectors, and other nucleic acid therapeutics for treatment. The present disclosure relates to the delivery of exogenous DNA sequences to a target cell, tissue, organ or organism, and modifications and methods for inhibiting (i.e., reducing or suppressing) an immune response (e.g., an innate immune response) to the same. Such modifications and methods for inhibiting (i.e., reducing or suppressing) an immune response (e.g., an innate immune response) can be used to, for example, enhance duration of transgene expression.

It has been unexpectedly discovered that an immune response (e.g., an innate immune response) to DNA transfer vector can be attenuated with the methods and related compositions provided herein. Hence, the methods and compositions can potentially increase the efficacy of treatment with viral transfer vectors and other therapeutic nucleic acid molecules and provide for long-term therapeutic benefits, even if the administration of the viral transfer vector or other nucleic acid therapeutics is repeated.

### I. Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, Pa., USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the terms, “administration,” “administering” and variants thereof refers to introducing a composition or agent (e.g., a therapeutic nucleic acid or an immunosuppressant as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. The introduction of a composition or agent into a subject is by electroporation. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

As used herein, an “effective amount” or “therapeutically effective amount” of an active agent or therapeutic agent, such as an immunosuppressant and/or therapeutic nucleic acid, is an amount sufficient to produce the desired effect, e.g., a normalization or reduction of immune response (e.g., innate immune response) and expression or inhibition of expression of a target sequence in comparison to the expression level detected in the absence of a therapeutic nucleic acid and/or immunosuppressant. Suitable assays for measuring expression of a target gene or target sequence include, e.g., examination of protein or RNA levels using techniques known to those of skill in the art such as dot blots, northern blots, in situ hybridization, ELISA, immunoprecipitation, enzyme function, as well as phenotypic assays known to those of skill in the art. However, dosage levels are based on a variety of factors, including the type of injury, the age, weight, sex, medical condition of the patient, the severity of the condition, the route of administration, and the particular active agent employed. Thus, the dosage regimen may vary widely, but can be determined routinely by a physician using standard methods. Additionally, the terms “therapeutic amount”, “therapeutically effective amounts” and “pharmaceutically effective amounts” include prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10^{th} Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, the terms “heterologous nucleotide sequence” and “transgene” are used interchangeably and refer to a nucleic acid of interest (other than a nucleic acid encoding a capsid polypeptide) that is incorporated into and may be delivered and expressed by a ceDNA vector as disclosed herein.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

As used herein, the term “interfering RNA” or “RNAi” or “interfering RNA sequence” includes single-stranded RNA (e.g., mature miRNA, ssRNAi oligonucleotides, ssDNAi oligonucleotides), double-stranded RNA (i.e., duplex RNA such as siRNA, Dicer-substrate dsRNA, shRNA, aiRNA, or pre-miRNA), a DNA-RNA hybrid (see, e.g., PCT Publication No. WO 2004/078941), or a DNA-DNA hybrid (see, e.g., PCT Publication No. WO 2004/104199) that is capable of reducing or inhibiting the expression of a target gene or sequence (e.g., by mediating the degradation or inhibiting the translation of mRNAs which are complementary to the interfering RNA sequence) when the interfering RNA is in the same cell as the target gene or sequence. Interfering RNA thus refers to the single-stranded RNA that is complementary to a target mRNA sequence or to the double-stranded RNA formed by two complementary strands or by a single, self-complementary strand. Interfering RNA may have substantial or complete identity to the target gene or sequence, or may comprise a region of mismatch (i.e., a mismatch motif). The sequence of the interfering RNA can correspond to the full-length target gene, or a subsequence thereof. Preferably, the interfering RNA molecules are chemically synthesized. The disclosures of each of the above patent documents are herein incorporated by reference in their entirety for all purposes.

Interfering RNA includes “small-interfering RNA” or “siRNA,” e.g., interfering RNA of about 15-60, 15-50, or 15-40 (duplex) nucleotides in length, more typically about 15-30, 15-25, or 19-25 (duplex) nucleotides in length, and is preferably about 20-24, 21-22, or 21-23 (duplex) nucleotides in length (e.g., each complementary sequence of the double-stranded siRNA is 15-60, 15-50, 15-40, 15-30, 15-25, or 19-25 nucleotides in length, preferably about 20-24, 21-22, or 21-23 nucleotides in length, and the double-stranded siRNA is about 15-60, 15-50, 15-40, 15-30, 15-25, or 19-25 base pairs in length, preferably about 18-22, 19-20, or 19-21 base pairs in length). siRNA duplexes may comprise 3′ overhangs of about 1 to about 4 nucleotides or about 2 to about 3 nucleotides and 5′ phosphate termini Examples of siRNA include, without limitation, a double-stranded polynucleotide molecule assembled from two separate stranded molecules, wherein one strand is the sense strand and the other is the complementary antisense strand; a double-stranded polynucleotide molecule assembled from a single stranded molecule, where the sense and antisense regions are linked by a nucleic acid-based or non-nucleic acid-based linker; a double-stranded polynucleotide molecule with a hairpin secondary structure having self-complementary sense and antisense regions; and a circular single-stranded polynucleotide molecule with two or more loop structures and a stem having self-complementary sense and antisense regions, where the circular polynucleotide can be processed in vivo or in vitro to generate an active double-stranded siRNA molecule. As used herein, the term “siRNA” includes RNA-RNA duplexes as well as DNA-RNA hybrids (see, e.g., PCT Publication No. WO 2004/078941).

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to an uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular inflammasome antagonist (e.g., any one or more of: an inhibitor of the NLRP3 inflammasome pathway, or an inhibitor of the AIM2 inflammasome pathway, or an inhibitor of caspase 1, or any combination thereof) is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “fusion protein” as used herein refers to a polypeptide which comprises protein domains from at least two different proteins. For example, a fusion protein may comprise (i) one an inflammasome antagonist (e.g., any one or more of: an inhibitor of the NLRP3 inflammasome pathway, or an inhibitor of the AIM2 inflammasome pathway, or an inhibitor of caspase 1, or any combination thereof) or fragment thereof and (ii) at least one non-Gene of interest (GOI) protein or alternatively, a different inflammasome antagonist protein. Fusion proteins encompassed herein include, but are not limited to, an antibody, or Fc or antigen-binding fragment of an antibody fused to an inflammasome antagonist (e.g., any one or more of: an inhibitor of the NLRP3 inflammasome pathway, or an inhibitor of the AIM2 inflammasome pathway, or an inhibitor of caspase 1, or any combination thereof), e.g., an extracellular domain of a receptor, ligand, enzyme or peptide. An inflammasome antagonist (e.g., any one or more of: an inhibitor of the NLRP3 inflammasome pathway, or an inhibitor of the AIM2 inflammasome pathway, or an inhibitor of caspase 1, or any combination thereof) or fragment thereof that is part of a fusion protein can be a monospecific antibody or a bispecific or multispecific antibody.

As used herein, the term “genomic safe harbor gene” or “safe harbor gene” refers to a gene or loci that a nucleic acid sequence can be inserted such that the sequence can integrate and function in a predictable manner (e.g., express a protein of interest) without significant negative consequences to endogenous gene activity, or the promotion of cancer. In some embodiments, a safe harbor gene is also a loci or gene where an inserted nucleic acid sequence can be expressed efficiently and at higher levels than a non-safe harbor site.

As used herein, the term “gene delivery” means a process by which foreign DNA is transferred to host cells for applications of gene therapy.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector. As used herein, the terms “treat,” “treating,” and/or “treatment” include abrogating, substantially inhibiting, slowing or reversing the progression of a condition, substantially ameliorating clinical symptoms of a condition, or substantially preventing the appearance of clinical symptoms of a condition, obtaining beneficial or desired clinical results. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s). Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition. By “decrease,” “decreasing,” “reduce,” or “reducing” of an immune response (e.g., an immune response (e.g., innate immune response)) by an immunosuppressant is intended to mean a detectable decrease of an immune response to a given immunosuppressant. The amount of decrease of an immune response by the immunosuppressant may be determined relative to the level of an immune response in the presence of an immunosuppressant. A detectable decrease can be about 5%, 10%, 15%, 20%, 25%, 30%, 35%, 40%, 45%, 50%, 55%, 60%, 65%, 70%, 75%, 80%, 85%, 90%, 95%, 100%, or more lower than the immune response detected in the presence of the immunosuppressant.

As used herein, the term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

As used herein, the term “lipid particle” includes a lipid formulation that can be used to deliver a therapeutic agent such as nucleic acid therapeutics and/or an immunosuppressant to a target site of interest (e.g., cell, tissue, organ, and the like). In preferred embodiments, the lipid particle of the invention is a nucleic acid containing lipid particle, which is typically formed from a cationic lipid, a non-cationic lipid, and optionally a conjugated lipid that prevents aggregation of the particle. In other preferred embodiments, a therapeutic agent such as a therapeutic nucleic acid may be encapsulated in the lipid portion of the particle, thereby protecting it from enzymatic degradation. In other preferred embodiments, an immunosuppressant can be optionally included in the nucleic acid containing lipid particles.

As used herein, the term “lipid encapsulated” can refer to a lipid particle that provides an active agent or therapeutic agent, such as a nucleic acid (e.g., a ceDNA), with full encapsulation, partial encapsulation, or both. In a preferred embodiment, the nucleic acid is fully encapsulated in the lipid particle (e.g., to form a nucleic acid containing lipid particle).

As used herein, the term “lipid conjugate” refers to a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine, and dilinoleoylphosphatidylcholine. Other compounds lacking in phosphorus, such as sphingolipid, glycosphingolipid families, diacylglycerols, and β-acyloxyacids, are also within the group designated as amphipathic lipids. Additionally, the amphipathic lipids described above can be mixed with other lipids including triglycerides and sterols.

As used herein, the term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

As used herein, the term “non-cationic lipid” refers to any amphipathic lipid as well as any other neutral lipid or anionic lipid.

As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

As used herein, the term “hydrophobic lipid” refers to compounds having apolar groups that include, but are not limited to, long-chain saturated and unsaturated aliphatic hydrocarbon groups and such groups optionally substituted by one or more aromatic, cycloaliphatic, or heterocyclic group(s). Suitable examples include, but are not limited to, diacylglycerol, dialkylglycerol, N—N-dialkylamino, 1,2-diacyloxy-3-aminopropane, and 1,2-dialkyl-3-aminopropane.

As used herein, the term “aqueous solution” refers to a composition comprising in whole, or in part, water.

As used herein, the term “organic lipid solution” refers to a composition comprising in whole, or in part, an organic solvent having a lipid.

As used herein, the term “systemic delivery” refers to delivery of lipid particles that leads to a broad biodistribution of an active agent such as an interfering RNA (e.g., siRNA) within an organism. Some techniques of administration can lead to the systemic delivery of certain agents, but not others. Systemic delivery means that a useful, preferably therapeutic, amount of an agent is exposed to most parts of the body. To obtain broad biodistribution generally requires a blood lifetime such that the agent is not rapidly degraded or cleared (such as by first pass organs (liver, lung, etc.) or by rapid, nonspecific cell binding) before reaching a disease site distal to the site of administration. Systemic delivery of lipid particles can be by any means known in the art including, for example, intravenous, subcutaneous, and intraperitoneal. In a preferred embodiment, systemic delivery of lipid particles is by intravenous delivery.

As used herein, the term “local delivery” refers to delivery of an active agent such as an interfering RNA (e.g., siRNA) directly to a target site within an organism. For example, an agent can be locally delivered by direct injection into a disease site such as a tumor or other target site such as a site of inflammation or a target organ such as the liver, heart, pancreas, kidney, and the like.

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found in the invention herein, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses parvoviruses and dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleotide sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. In some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (TRS) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change in one or more of A, C, C′, B, B′ regions in the ITR, and can result in a change in the three-dimensional spatial organization (i.e. its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. In one embodiment, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). In some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are wild-type or mutated (e.g., modified relative to wild-type) dependoviral ITR sequences and are inverse complements across their full length. In one non-limiting example, both ITRs are wild type ITRs sequences from AAV2. In another example, neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the a modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. In some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g. AAV1, 2, 3, 4, 5, 6, 7, 8, 9, 10, 11, and 12) such as the combination of AAV2 and AAV6, with the modification in one ITR reflected in the corresponding position in the cognate ITR from a different serotype. In one embodiment, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. In one embodiment, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. In some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. In some embodiments, ceDNA spacer regions keep two functional elements at a desired distance for optimal functionality. In some embodiments, ceDNA spacer regions provide or add to the genetic stability of the ceDNA genome within e.g., a plasmid or baculovirus. In some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference.

As used herein, the terms “ceDNA vector” and “ceDNA” are used interchangeably and refer to a closed-ended DNA vector comprising at least one terminal palindrome. In some embodiments, the ceDNA comprises two covalently-closed ends.

As used herein, the term “neDNA” or “nicked ceDNA” refers to a closed-ended DNA having a nick or a gap of 1-100 base pairs in a stem region or spacer region 5′ upstream of an open reading frame (e.g., a promoter and transgene to be expressed).

As used herein, the terms “gap” and “nick” are used interchangeably and refer to a discontinued portion of synthetic DNA vector of the present invention, creating a stretch of single stranded DNA portion in otherwise double stranded ceDNA. The gap can be 1 base-pair to 100 base-pair long in length in one strand of a duplex DNA. Typical gaps, designed and created by the methods described herein and synthetic vectors generated by the methods can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 bp long in length. Exemplified gaps in the present disclosure can be 1 bp to 10 bp long, 1 to 20 bp long, 1 to 30 bp long in length.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′ (SEQ ID NO: 39), an RBS sequence identified in AAV2. Any known RBS sequence may be used in the embodiments of the invention, including other known AAV RBS sequences and other naturally known or synthetic RBS sequences. Without being bound by theory it is thought that he nuclease domain of a Rep protein binds to the duplex nucleotide sequence GCTC, and thus the two known AAV Rep proteins bind directly to and stably assemble on the duplex oligonucleotide, 5′-(GCGC)(GCTC)(GCTC)(GCTC)-3′ (SEQ ID NO: 39). In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. In some embodiments, a TRS minimally encompasses a non-base-paired thymidine. In some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′ (SEQ ID NO: 804), the hexanucleotide sequence identified in AAV2. Any known TRS sequence may be used in the embodiments of the invention, including other known AAV TRS sequences and other naturally known or synthetic TRS sequences such as AGTT (SEQ ID NO: 085), GGTTGG (SEQ ID NO: 806), AGTTGG (SEQ ID NO: 807), AGTTGA (SEQ ID NO: 808), and other motifs such as RRTTRR (SEQ ID NO: 809).

As used herein, the terms “sense” and “antisense” refer to the orientation of the structural element on the polynucleotide. The sense and antisense versions of an element are the reverse complement of each other.

As used herein, the term “synthetic AAV vector” and “synthetic production of AAV vector” refers to an AAV vector and synthetic production methods thereof in an entirely cell-free environment.

As used herein, “reporters” refer to proteins that can be used to provide detectable read-outs. Reporters generally produce a measurable signal such as fluorescence, color, or luminescence. Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. In some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a gene of interest, such as an inflammasome antagonist (e.g., inhibitor of one or more of NLRP3 and/or AIM2 inflammasome pathway, or a caspase 1 inhibitor). Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to, homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. In one embodiment, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. In some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. In some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background.

The term “enhancer” as used herein refers to a cis-acting regulatory sequence (e.g., 50-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, in some embodiments, an enhancer can be one naturally associated with a nucleic acid sequence, located either downstream or upstream of that sequence.

In some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. In some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. In some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The phrase “operably linked” refers to a juxtaposition wherein the components so described are in a relationship permitting them to function in their intended manner. For instance, a promoter is operably linked to a coding sequence if the promoter affects its transcription or expression. An “expression cassette” includes a heterologous DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present invention, is provided. Usually the animal is a vertebrate such as, but not limited to a primate, rodent, domestic animal or game animal Primates include but are not limited to, chimpanzees, cynomologous monkeys, spider monkeys, and macaques, e.g., Rhesus. Rodents include mice, rats, woodchucks, ferrets, rabbits and hamsters. Domestic and game animals include, but are not limited to, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments of the aspects described herein, the subject is a mammal, e.g., a primate or a human A subject can be male or female. Additionally, a subject can be an infant or a child. In some embodiments, the subject can be a neonate or an unborn subject, e.g., the subject is in utero. Preferably, the subject is a mammal. The mammal can be a human, non-human primate, mouse, rat, dog, cat, horse, or cow, but is not limited to these examples. Mammals other than humans can be advantageously used as subjects that represent animal models of diseases and disorders. In addition, the methods and compositions described herein can be used for domesticated animals and/or pets. A human subject can be of any age, gender, race or ethnic group, e.g., Caucasian (white), Asian, African, black, African American, African European, Hispanic, Mideastern, etc. In some embodiments, the subject can be a patient or other subject in a clinical setting. In some embodiments, the subject is already undergoing treatment. In some embodiments, the subject is an embryo, a fetus, neonate, infant, child, adolescent, or adult. In some embodiments, the subject is a human fetus, human neonate, human infant, human child, human adolescent, or human adult. In some embodiments, the subject is an animal embryo, or non-human embryo or non-human primate embryo. In some embodiments, the subject is a human embryo.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. In some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleotide sequence encoding a fusion variant polypeptide.

A “vector” or “expression vector” is a replicon, such as plasmid, bacmid, phage, virus, virion, or cosmid, to which another DNA segment, i.e. an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. In some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The phrase “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, Lesch Nyhan syndrome, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

An “inhibitory polynucleotide” as used herein refers to a DNA or RNA molecule that reduces or prevents expression (transcription or translation) of a second (target) polynucleotide. Inhibitory polynucleotides include antisense polynucleotides, ribozymes, and external guide sequences. The term “inhibitory polynucleotide” further includes DNA and RNA molecules, e.g., RNAi that encode the actual inhibitory species, such as DNA molecules that encode ribozymes.

As used herein, “gene silencing” or “gene silenced” in reference to an activity of an RNAi molecule, for example a siRNA or miRNA refers to a decrease in the mRNA level in a cell for a target gene (e.g. NLRP3, AIM2 or caspase-1 mRNA) by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene. By way of an example only, in some embodiments RNAi agents which serve to inhibit or gene silence are useful in the methods, kits and compositions disclosed herein, e.g., to inhibit the immune response (e.g., the innate immune response).

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±1%. The present invention is further explained in detail by the following examples, but the scope of the invention should not be limited thereto.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

### II. Nucleic Acids

Nucleic acids are large, highly charged, rapidly degraded and cleared from the body, and offer generally poor pharmacological properties because they are recognized as a foreign matter to the body and become a target of an immune response (e.g., innate immune response). Hence, certain nucleic acids, such as therapeutic nucleic acids or nucleic acids used for research purposes (e.g., antisense oligonucleotide or viral vectors) can often trigger immune responses in vivo. The present disclosure provides pharmaceutical compositions and methods that may ameliorate, reduce or eliminate such immune responses and enhance efficacy of the nucleic acids by increasing expression levels through maximizing the durability of the nucleic acid in a reduced immune-responsive state in a subject recipient. This may also minimize any potential adverse events that may lead to an organ damage or other toxicity in the course of gene therapy. Many of the compositions and methods provided herein relate to the administration of a specific inhibitor of the immune response (e.g., innate immune response) in conjunction with a nucleic acid (e.g., a therapeutic nucleic acid or a nucleic acid used for research purposes), thereby reducing the immune response (e.g., innate immune response) triggered by the presence of the nucleic acid.

The immunogenic/immunostimulatory nucleic acids can include both deoxyribonucleic acids and ribonucleic acids. For deoxyribonucleic acids (DNA), a particular sequence or motif has been shown to induce immune stimulation in mammals. These sequence or motifs include, but are not limited to, CpG motifs, pyrimidine-rich sequences, and palindrome sequences. CpG motifs in deoxyribonucleic acid are often recognized by the endosomal toll-like receptor 9 (TLR-9) which, in turn, triggers both the innate immune stimulatory pathway and the acquired immune stimulatory pathway. Certain immunostimulatory ribonucleic acid (RNA) sequences bind to toll-like receptor 6 and 7 (TLR-6 and TLR-7) and are believed to activate proinflammatory response through the immune response (e.g., innate immune response). Furthermore, double-stranded RNA can be often immunostimulatory because of its binding to TLR-3. Therefore, foreign nucleic acid molecules, either pathogen derived or therapeutic in their origin, can be highly immunogenic in vivo.

The characterization and development of nucleic acid molecules for potential therapeutic use in conjunction with antagonists of the immune response (e.g., innate immune response) are provided herein. In some embodiments, chemical modification of oligonucleotides for the purpose of altered and improved in vivo properties (delivery, stability, life-time, folding, target specificity), as well as their biological function and mechanism that directly correlate with therapeutic application, are described where appropriate.

Illustrative therapeutic nucleic acids of the present disclosure that can be immunostimulatory and require use of immunosuppressants disclosed herein can include, but are not limited to, minigenes, plasmids, minicircles, small interfering RNA (siRNA), microRNA (miRNA), antisense oligonucleotides (ASO), ribozymes, closed ended double stranded DNA (e.g., ceDNA, CELiD, linear covalently closed DNA (“ministring”), doggybone (dbDNA™), protelomere closed ended DNA, or dumbbell linear DNA), dicer-substrate dsRNA, small hairpin RNA (shRNA), asymmetrical interfering RNA (aiRNA), mricroRNS (miRNA), mRNA, tRNA, rRNA, and DNA viral vectors, viral RNA vector, and any combination thereof.

siRNA or miRNA that can downregulate the intracellular levels of specific proteins through a process called RNA interference (RNAi) are also contemplated by the present invention to be nucleic acid therapeutics. After siRNA or miRNA is introduced into the cytoplasm of a host cell, these double-stranded RNA constructs can bind to a protein called RISC. The sense strand of the siRNA or miRNA is removed by the RISC complex. The RISC complex, when combined with the complementary mRNA, cleaves the mRNA and release the cut strands. RNAi is by inducing specific destruction of mRNA that results in downregulation of a corresponding protein.

Antisense oligonucleotides (ASO) and ribozymes that inhibit mRNA translation into protein can be nucleic acid therapeutics. For antisense constructs, these single stranded deoxy nucleic acids have a complementary sequence to the sequence of the target protein mRNA, and Watson—capable of binding to the mRNA by Crick base pairing. This binding prevents translation of a target mRNA, and/or triggers RNaseH degradation of the mRNA transcript. As a result, the antisense oligonucleotide has increased specificity of action (i.e., down-regulation of a specific disease-related protein).

In any of the methods provided herein, the therapeutic nucleic acid can be a therapeutic RNA. The therapeutic RNA can be an inhibitor of mRNA translation, agent of RNA interference (RNAi), catalytically active RNA molecule (ribozyme), transfer RNA (tRNA) or an RNA that binds an mRNA transcript (ASO), protein or other molecular ligand (aptamer). In any of the methods provided herein, the agent of RNAi can be a double-stranded RNA, single-stranded RNA, micro RNA, short interfering RNA, short hairpin RNA, or a triplex-forming oligonucleotide.

According to some embodiments, the therapeutic nucleic acid is a closed ended double stranded DNA, e.g., a ceDNA. According to some embodiments, the expression and/or production of a therapeutic protein in a cell is from a non-viral DNA vector, e.g., a ceDNA vector. A distinct advantage of ceDNA vectors for expression of a therapeutic protein over traditional AAV vectors, and even lentiviral vectors, is that there is no size constraint for the heterologous nucleic acid sequences encoding a desired protein. Thus, even a large therapeutic protein can be expressed from a single ceDNA vector. Thus, ceDNA vectors can be used to express a therapeutic protein in a subject in need thereof.

In general, a ceDNA vector for expression of a therapeutic protein as disclosed herein, comprises in the 5′ to 3′ direction: a first adeno-associated virus (AAV) inverted terminal repeat (ITR), a nucleotide sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

### III. ceDNA Vectors

According to some aspects, the disclosure provides non-viral, capsid-free DNA vectors with covalently-closed ends (ceDNA vector) administered in conjunction with rapamycin or rapamycin analogs. In some embodiments, the rapamycin or rapamycin analog is present in a super-saturated amount in a synthetic nanocarrier as described in WO 2016/073799. In some embodiments, the ceDNA vector is also present in the same nanocarrier.

According to some aspects, the disclosure provides non-viral, capsid-free DNA vectors with covalently-closed ends (ceDNA) administered in conjunction with one or more TLR9 antagonists. Also provided herein are ceDNA constructs comprising sequences encoding, in part, one or more TLR9 inhibitory oligonucleotides.

According to some aspects, the disclosure provides non-viral, capsid-free DNA vectors with covalently-closed ends (ceDNA) administered in conjunction with one or more cGAS antagonists. Also provided herein are ceDNA constructs comprising sequences encoding, in part, one or more cGAS inhibitory RNAs or proteins.

According to some aspects, the disclosure provides non-viral, capsid-free DNA vectors with covalently-closed ends (ceDNA) administered in conjunction with one or more inflammasome antagonists (e.g., any one or more of: an inhibitor of the NLRP3 inflammasome pathway, or an inhibitor of the AIM2 inflammasome pathway, or an inhibitor of caspase 1, or any combination thereof). Also provided herein are ceDNA constructs comprising sequences encoding, in part, one or more inflammasome antagonists (e.g., any one or more of: an inhibitor of the NLRP3 inflammasome pathway, or an inhibitor of the AIM2 inflammasome pathway, or an inhibitor of caspase 1, or any combination thereof).

As one will appreciate, the ceDNA vector technologies described herein can be adapted to any level of complexity or can be used in a modular fashion, where expression of different components of an inhibitor of the immune response (e.g., the innate immune response), such as those described herein, e.g. can be controlled in an independent manner. For example, it is specifically contemplated that the ceDNA vector technologies designed herein can be as simple as using a single ceDNA vector to express a single heterologous gene sequence (e.g., a single inhibitor of the immune response (e.g., the innate immune response), such as those described herein, e.g. in) or can be as complex as using multiple ceDNA vectors, where each vector expresses multiple inhibitors of the immune response (e.g., the innate immune response), such as those described herein, e.g., or a nucleic acid sequence encoding or one or more inhibitors of the immune response (e.g., the innate immune response), such as those described herein, and e.g. associated co-factors or accessory proteins that are each independently controlled by different promoters.

In one embodiment, a single ceDNA vector can be used to express a single component of an inflammasome antagonist (e.g., any one or more of: an inhibitor of the NLRP3 inflammasome pathway, or an inhibitor of the AIM2 inflammasome pathway, or an inhibitor of caspase 1, or any combination thereof). Alternatively, a single ceDNA vector can be used to express multiple components (e.g., at least 2), e.g., it can express two or more inhibitors of the NLRP3 inflammasome pathway, and/or two or more inhibitors of the AIM2 inflammasome pathway, and/or two or more inhibitors of caspase 1, or any combination thereof) under the control of a single promoter (e.g., a strong promoter), optionally using an IRES sequence(s) to ensure appropriate expression of each of the components, e.g., co-factors or accessory proteins.

Also contemplated herein, in another embodiment, is a single ceDNA vector comprising at least two inserts, where the expression of each insert is under the control of its own promoter. The promoters can include multiple copies of the same promoter, multiple different promoters, or any combination thereof. As one of skill in the art will appreciate, it is often desirable to express multiple inflammasome antagonists) at different expression levels, thus controlling the stoichiometry of the individual components expressed to ensure efficient expression and, if a protein, protein folding and combination in the cell.

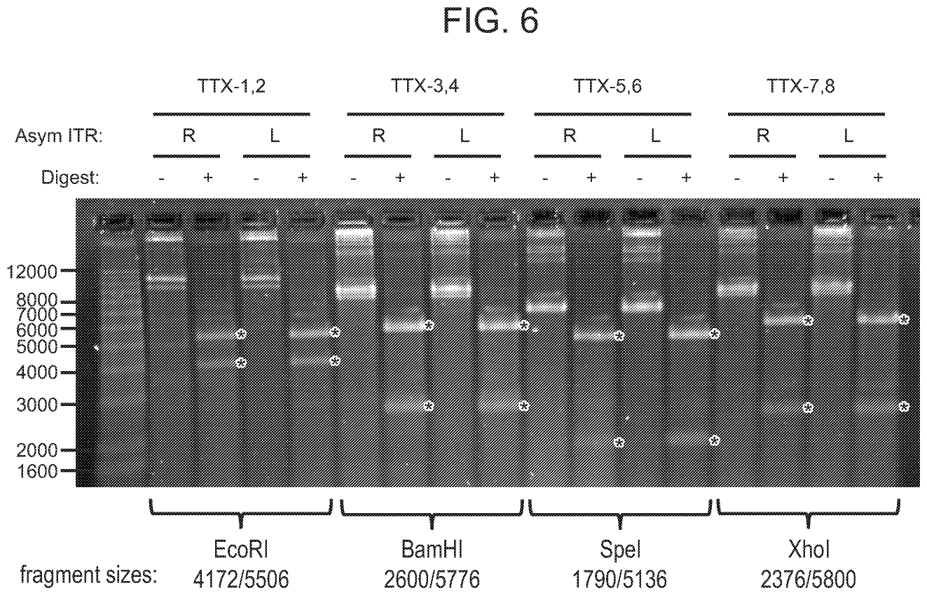

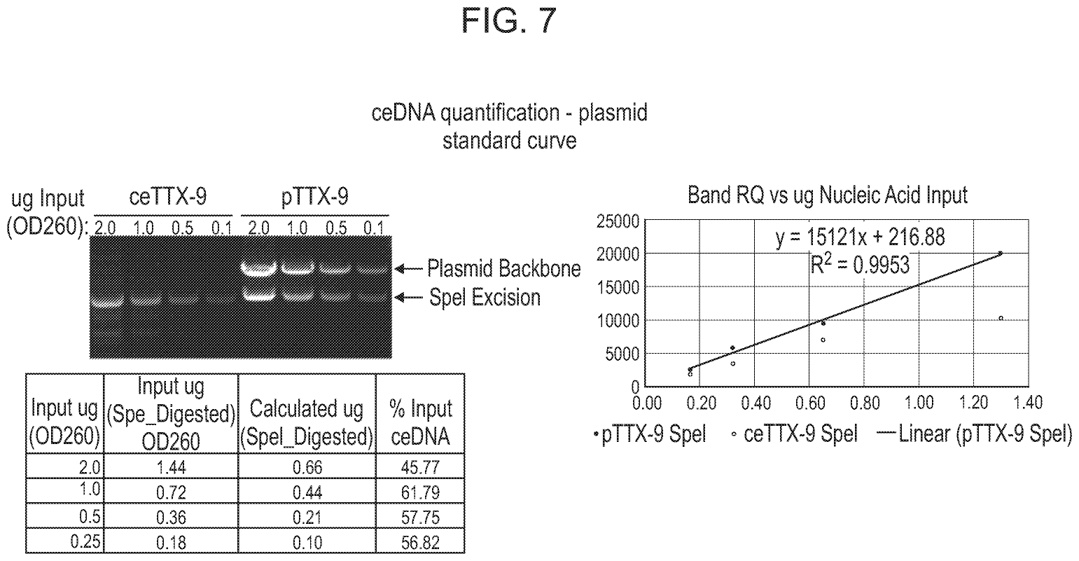

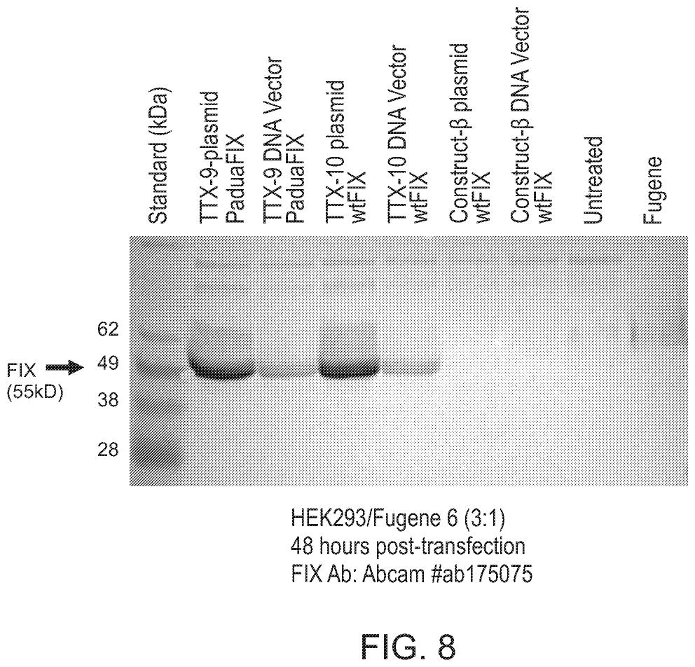

According to some embodiments, synthetic ceDNA is produced via excision from a double-stranded DNA molecule. Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. In some embodiments, the double stranded DNA construct is a ceDNA plasmid, e.g., see, e.g., FIG. 6 in International patent application PCT/US2018/064242, filed Dec. 6, 2018).

In some embodiments, a construct to make a ceDNA vector comprises a regulatory switch as described herein.

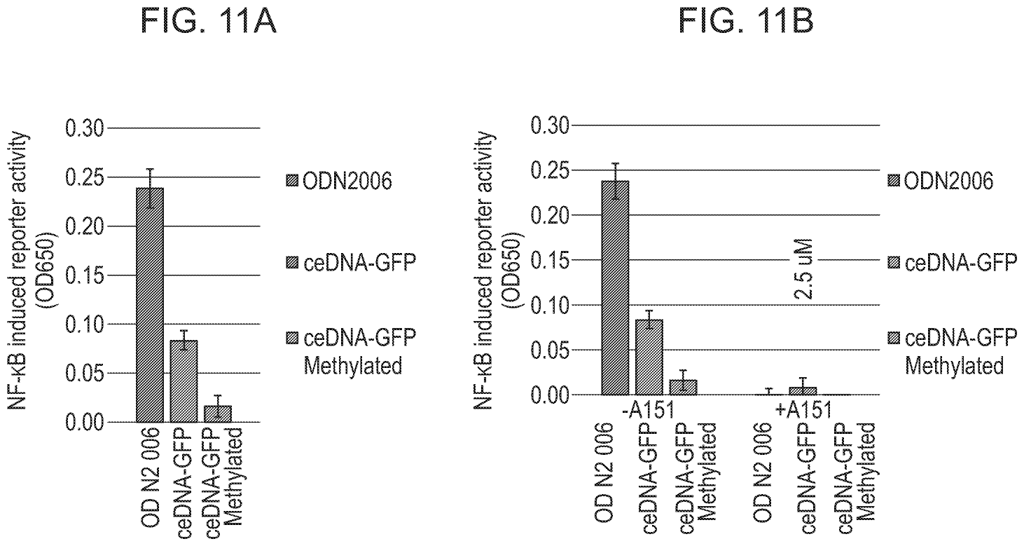

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

An exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

Additional variations of ceDNA vector technologies can be envisioned by one of skill in the art or can be adapted from protein production methods using conventional vectors.

The non-viral capsid free DNA vectors can be produced in permissive host cells from an expression construct (e.g., a plasmid, a Bacmid, a baculovirus, or an integrated cell-line) e.g., see the Examples disclosed in International Patent Application PCT/US18/49996 filed on Sep. 7, 2018, or using synthetic production, e.g., see the Examples disclosed in International Patent Application PCT/US19/14122, filed Dec. 6, 2018, each of which are incorporated herein in their entirety by reference. In some embodiments, the ceDNA vectors useful in the methods and compositions as disclosed herein comprise a heterologous nucleic acid, e.g. a transgene positioned between two inverted terminal repeat (ITR) sequences. In some embodiments, at least one of the ITRs is modified by deletion, insertion, and/or substitution as compared to a wild-type ITR sequence (e.g. AAV ITR); and at least one of the ITRs comprises a functional terminal resolution site (TRS) and a Rep binding site. In one embodiment, at least one of the ITRs has at least one polynucleotide deletion, insertion, or substitution with respect to a corresponding AAV ITR (e.g. SEQ ID NO:1, or SEQ ID NO:51, for wild type AAV2) to induce replication of the DNA vector in a host cell in the presence of Rep protein. As discussed above, any ITR can be used. For exemplary purposes, the ITRs in the ceDNA constructs in Table 1A are a modified ITR and a WT ITR. However, encompassed herein are ceDNA vectors that contain a heterologous nucleic acid sequence (e.g., a transgene) positioned between two inverted terminal repeat (ITR) sequences, where the ITR sequences can be an asymmetrical ITR pair or a symmetrical- or substantially symmetrical ITR pair, as these terms are defined herein. A ceDNA vector comprising a NLS as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

In some embodiments, the methods and compositions described herein relate to the use of an inhibitor of the immune response (e.g., the innate immune response) as disclosed herein for co-administration with any ceDNA vector, including but not limited to, a ceDNA vector comprising asymmetric ITRS as disclosed in International Patent Application PCT/US18/49996, filed on Sep. 7, 2018 (see, e.g., Examples 1-4); a ceDNA vector for gene editing as disclosed on the International Patent Application PCT/US18/64242 filed on Dec. 6, 2018 (see, e.g., Examples 1-7), or a ceDNA vector for production of antibodies or fusion proteins, as disclosed in the International Patent Application PCT/US19/18016, filed on Feb. 14, 2019, (e.g., see Examples 1-4), or a ceDNA vector for controlled transgene expression, as disclosed in International Patent Application PCT/US19/18927 filed on Feb. 22, 2019, each of which are incorporated herein in their entirety by reference. In some embodiments, it is also envisioned that the methods and compositions described herein using an inhibitor of the immune response (e.g., innate immune response) as disclosed herein can be used with a synthetically produced ceDNA vector, e.g., a ceDNA vector produced in a cell free or insect-free system of ceDNA production, as disclosed in International Application PCT/US19/14122, filed on Jan. 18, 2019, incorporated by reference in its entirety herein.

The ceDNA vector is preferably duplex, or self-complementary, over at least a portion of the molecule, e.g. the transgene. The ceDNA vector has covalently closed ends, and thus is preferably resistant to exonuclease digestion (e.g. Exo I or Exo III) for over an hour at 37° C. The presence of Rep protein in the host cells (e.g. insect cells or mammalian cells) promotes replication of the ceDNA vector polynucleotide template that has the modified ITR inducing production of non-viral capsid free DNA vector with covalently closed ends. The covalently closed ended molecule continues to accumulate in permissive cells through replication and is preferably sufficiently stable over time in the presence of Rep protein under standard replication conditions, e.g. to accumulate at yields of at least 1 pg/cell, preferably at least 2 pg/cell, preferably at least 3 pg/cell, more preferably at least 4 pg/cell, even more preferably at least 5 pg/cell.

In particular, in one embodiment, DNA vectors are produced by providing cells (e.g. insect cells or mammalian cells e.g. 293 cells etc.) harboring a polynucleotide vector template (e.g., expression construct) that comprises two different ITRs (e.g. AAV ITRs) and a nucleotide sequence of interest (a heterologous nucleic acid, expression cassette) positioned between the ITRs, wherein at least one of the ITRs is a modified ITR comprising an insertion, substitution, or deletion relative to the other ITR. The polynucleotide vector template described herein contains at least one functional ITR that comprises a Rep-binding site (RBS; e.g. 5′-GCGCGCTCGCTCGCTC-3′ for AAV2) and a functional terminal resolution site (TRS; e.g. 5′-AGTT). The cells do not express viral capsid proteins and the polynucleotide vector template is devoid of viral capsid coding sequences.

In the presence of Rep, the vector polynucleotide template having at least one modified ITR replicates to produce ceDNA vector. The ceDNA vector production undergoes two steps: first, excision (“rescue”) of template from the vector backbone (e.g. plasmid, bacmid, genome etc.) via Rep proteins, and second, Rep mediated replication of the excised vector genome. Rep proteins and Rep binding sites of the various AAV serotypes are well known to those of skill in the art One of skill in the art understands to choose a Rep protein from a serotype that binds to and replicates the functional ITR.

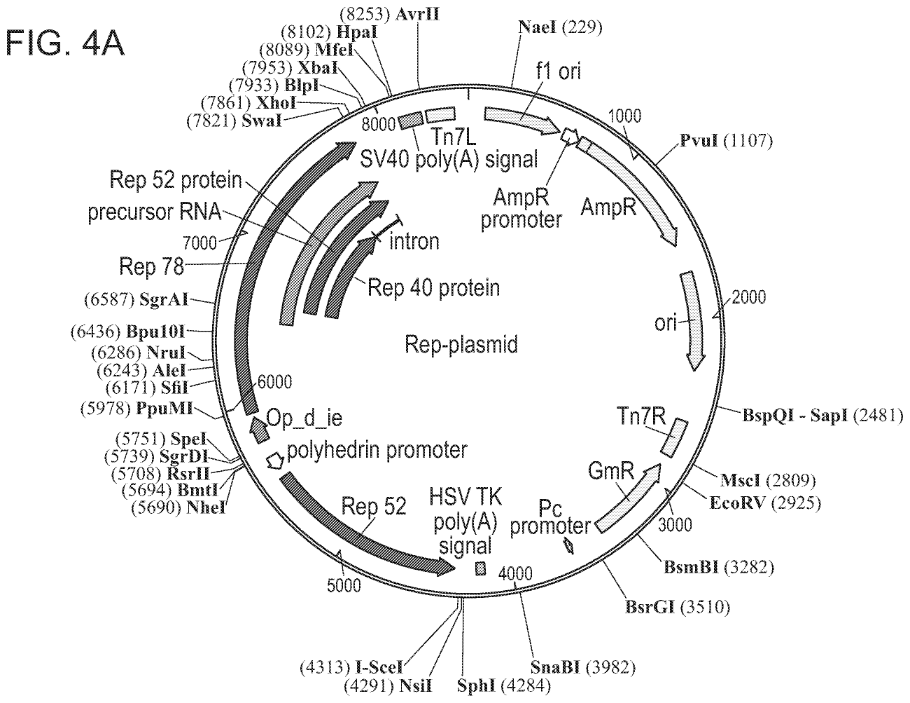

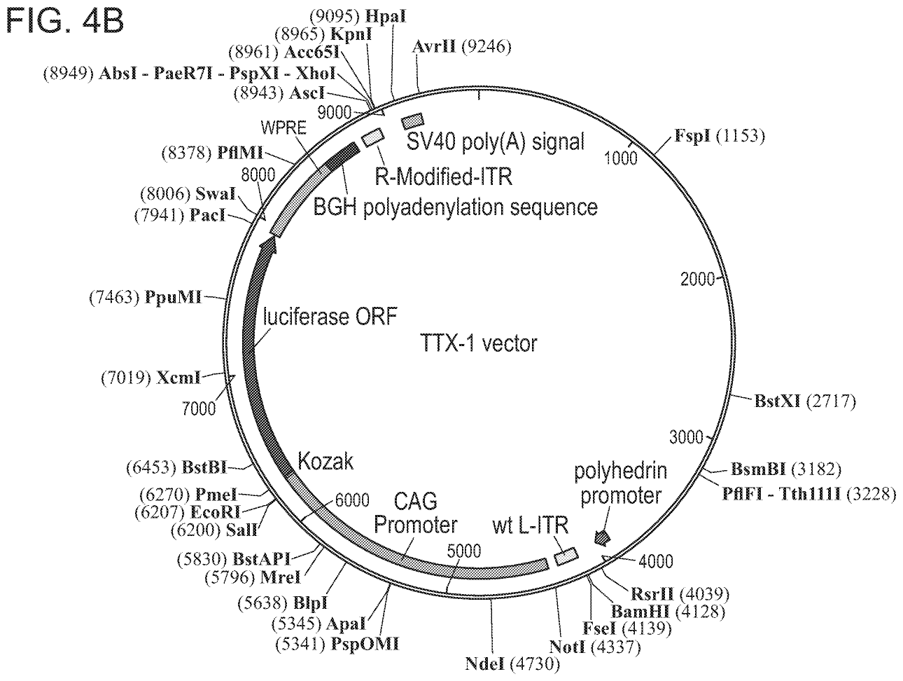

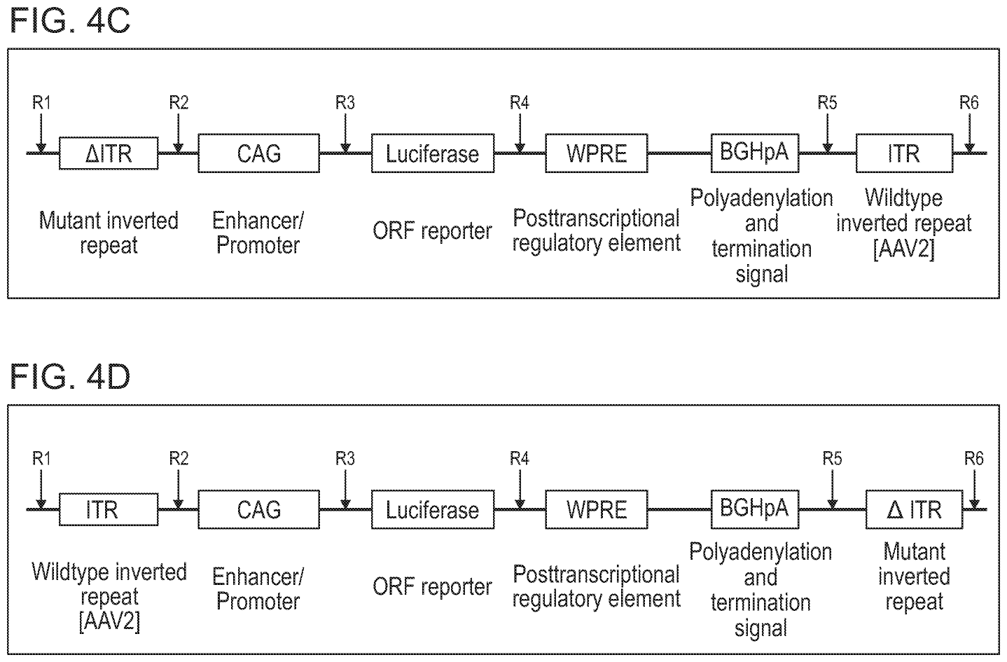

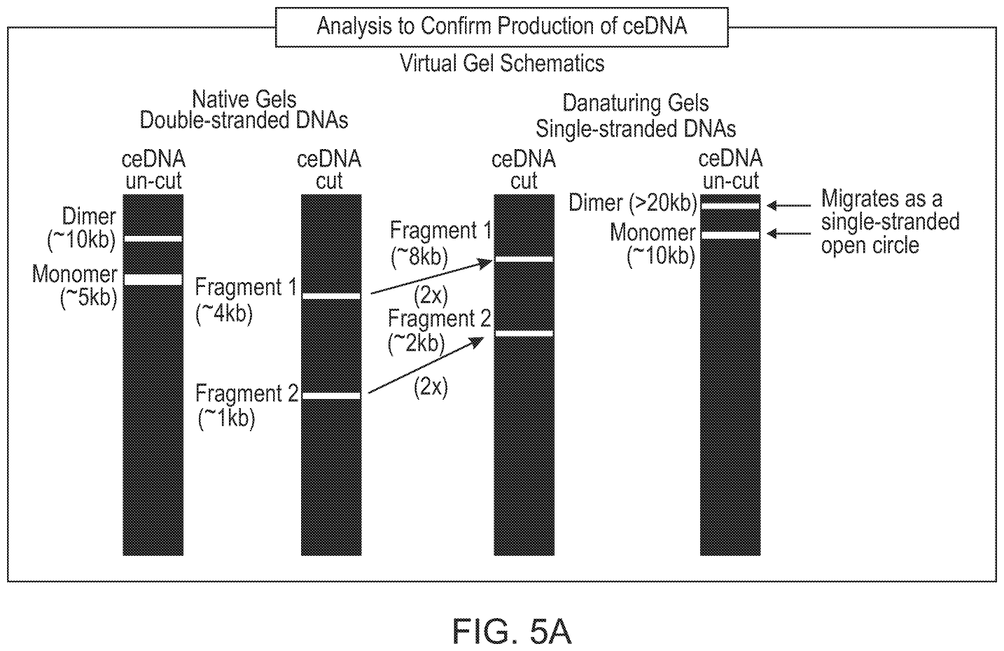

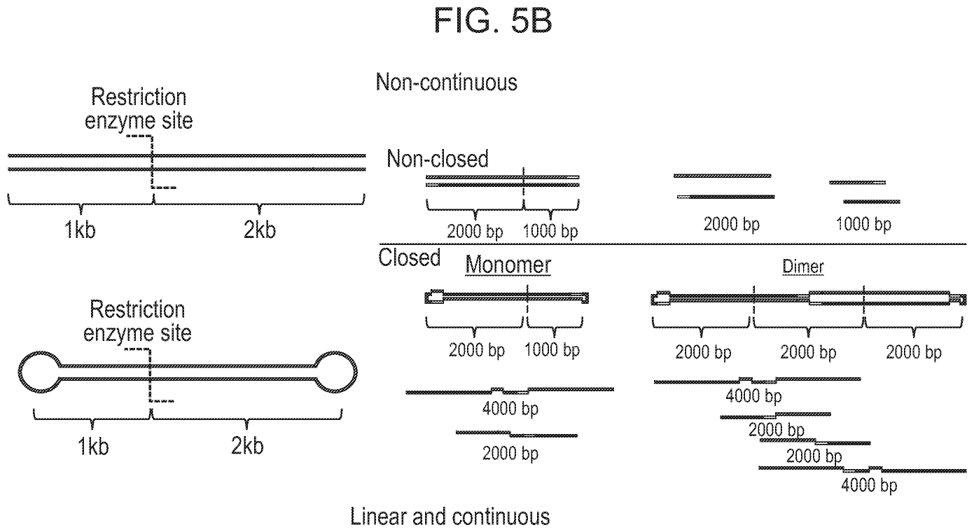

The cells harboring the vector polynucleotide either already contain Rep (e.g. a cell line with inducible rep), or are transduced with a vector that contains Rep and are then grown under conditions permitting replication and release of ceDNA vector. The ceDNA vector DNA is then harvested and isolated from the cells. The presence of the capsid-free, non-viral DNA ceDNA vector can be confirmed by digesting the vector DNA isolated from the cells with a restriction enzyme having a single recognition site on the DNA vector and analyzing the digested DNA material on a non-denaturing gel to confirm the presence of characteristic bands of linear and continuous DNA as compared to linear and non-continuous DNA. For example, FIG. 6 is a gel confirming the production of ceDNA vector from multiple TTX plasmid constructs using one embodiment for producing these vectors described in the Examples. The ceDNA vector is confirmed by a characteristic band pattern in the gel, as discussed with respect to FIG. 4D. FIG. 5A and FIG. 5B are drawings that illustrate one embodiment for identifying the presence of the close ended ceDNA vectors produced by the processed herein.

The vector polynucleotide expression template (e.g. TTX-plasmid, Bacmid etc.), and/or ii) a polynucleotide that encodes Rep can be introduced into cells using any means well known to those of skill in the art, including but not limited to transfection (e.g. calcium phosphate, nanoparticle, or liposome), or introduction by viral vectors, e.g. HSV or baculovirus. For example, the vector polynucleotide expression construct template used for generating the ceDNA vectors of the present invention can be a plasmid (e.g., TTX-plasmids, e.g. see FIG. 4B), a Bacmid (e.g., TTX-bacmid), and/or a baculovirus (e.g., TTX-baculovirus). In one embodiment, the TTX-plasmid comprises a restriction cloning site (e.g. SEQ ID NO: 7) operably positioned between the ITRs where the heterologous nucleic acid (e.g. expression cassette comprising a reporter gene or a therapeutic nucleic acid) can be inserted.

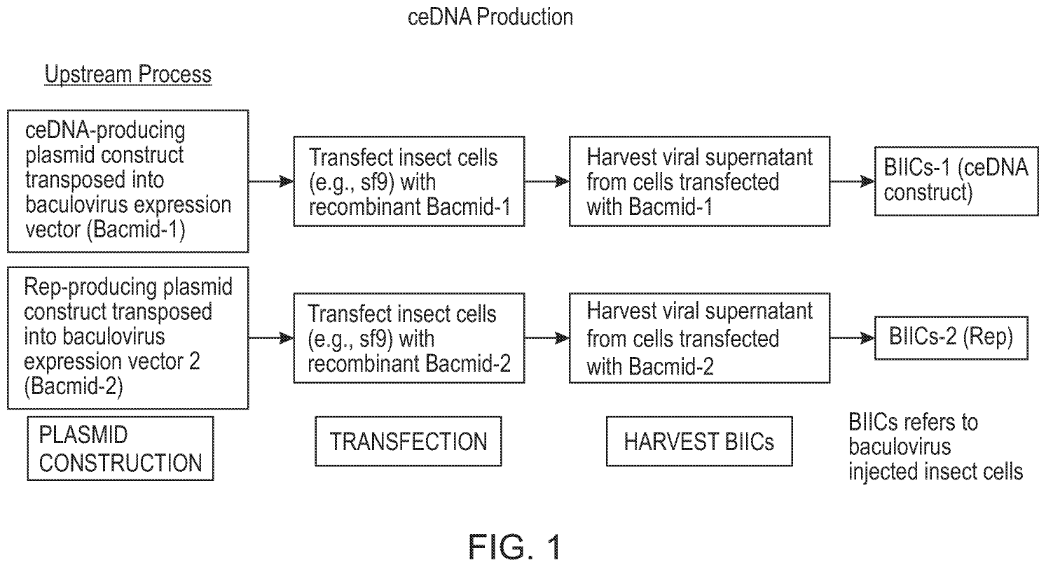

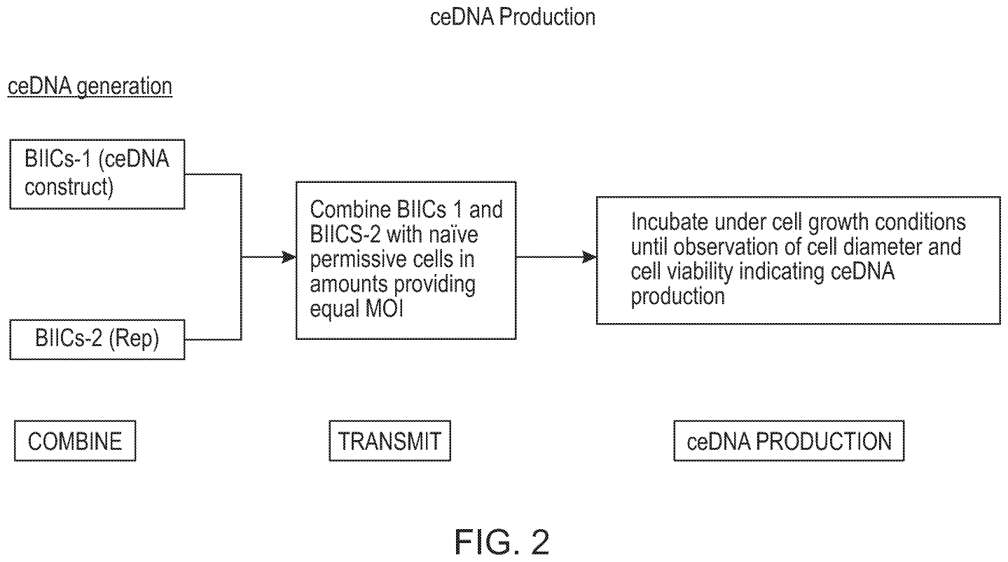

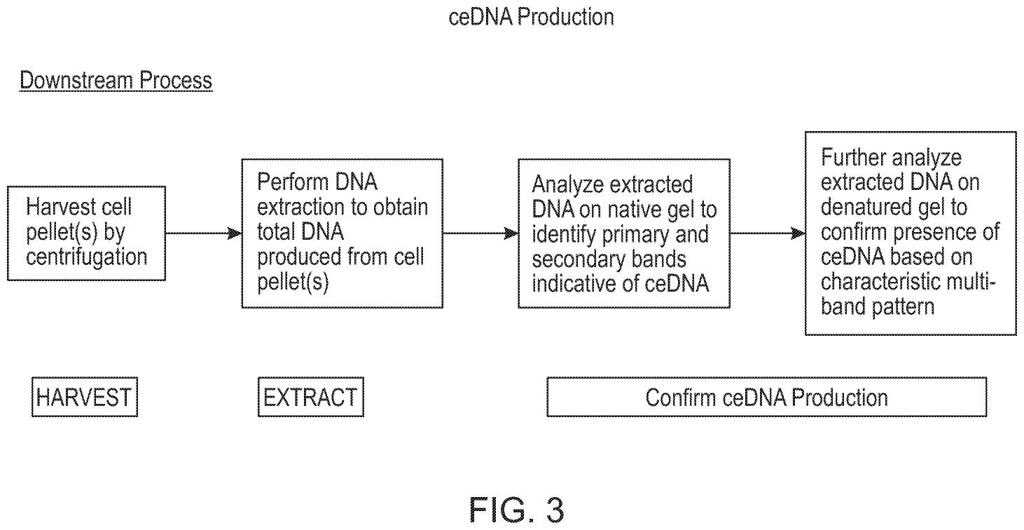

In one preferred embodiment, the host cells used to make the ceDNA vectors described herein are insect cells. In another preferred embodiment, baculovirus is used to deliver both the polynucleotide that encodes Rep protein and the non-viral DNA vector polynucleotide expression construct template for ceDNA vector. Examples of such processes for obtaining and isolating ceDNA vectors are described in FIGS. 1-33.

In yet another aspect, the invention provides for host cell lines that have stably integrated the DNA vector polynucleotide expression template (ceDNA vector template) described herein, into their own genome for use in production of the non-viral DNA vector. Methods for producing such cell lines are described in Lee, L. et al. (2013) Plos One 8(8): e69879, which is herein incorporated by reference in its entirety. Preferably, the Rep protein (e.g. as described in Example 1) is added to host cells at an MOI of 3. In one embodiment, the host cell line is an invertebrate cell line, preferably insect Sf9 cells. When the host cell line is a mammalian cell line, preferably 293 cells the cell lines can have polynucleotide vector template stably integrated, and a second vector, such as herpes virus can be used to introduce Rep protein into cells, allowing for the excision and amplification of ceDNA vector in the presence of Rep.

Preferably, the ceDNA contains one or more functional ITR polynucleotide sequences that include a Rep-binding site (RBS; 5′-GCGCGCTCGCTCGCTC-3′ for AAV2, SEQ ID NO: 39) and a terminal resolution site (TRS; 5′-AGTT).

The capsid-free ceDNA vectors can be produced from expression constructs (e.g., TTX-plasmids, TTX-Bacmids, TTX-baculovirus) that further include a specific combination of cis-regulatory elements such as WHP posttranscriptional regulatory element (WPRE) and BGH polyA. Suitable expression cassettes for use in expression constructs are not limited by the packaging constraint imposed by the viral capsid. Expression cassettes of the present disclosure include a promoter, which can influence overall expression levels as well as cell-specificity. For transgene expression, they can include a highly active virus-derived immediate early promoter. Expression cassettes can contain tissue-specific eukaryotic promoter to limit transgene expression to specific cell types and reduce toxic effects and immune responses resulting from unregulated, ectopic expression. In some embodiments, an expression cassette can contain a synthetic regulatory element, such as CAG promoter (SEQ ID NO: 3). The CAG promoter includes (i) the cytomegalovirus (CMV) early enhancer element (e.g., SEQ ID NO: 309), (ii) the promoter, the first exon and the first intron of chicken beta-actin gene, and (iii) the splice acceptor of the rabbit beta-globin gene. Alternatively, for example expression cassette can contain an Alpha-1-antitrypsin (AAT) promoter (e.g., SEQ ID NO: 4), a liver specific (LP1) promoter (e.g., SEQ ID NO: 5), or HAAT promoter (e.g., SEQ ID NO: 135) or Human elongation factor-1 alpha (EF1-α) promoter (SEQ ID NO: 6) or a EF1-α fragment (SEQ ID NO: 66), or a MND promoter (SEQ ID NO: 70). In some embodiments, the expression cassette includes one or more constitutive promoters, for example, the retroviral Rous sarcoma virus (RSV) LTR promoter (optionally with the RSV enhancer), cytomegalovirus (CMV) immediate early promoter (optionally with the CMV enhancer), or the like. Alternatively, an inducible promoter, a native promoter for a transgene, a tissue-specific promoter, or various promoters known in the art can be used. In one embodiment, the endogenous or native promoter for the gene coding sequence is used in the expression cassette.

Inducible gene editing using ceDNA vectors can be performed using the methods described in e.g., Dow et al. Nat Biotechnol 33:390-394 (2015); Zetsche et al. Nat Biotechnol 33:139-42 (2015); Davis et al. Nat Chem Biol 11:316-318 (2015); Polstein et al. Nat Chem Biol 11:198-200 (2015); and/or Kawano et al. Nat Commun 6:6256 (2015), the contents of each of which are incorporated herein by reference in their entirety. The expression cassettes can also include a post-transcriptional element, in particular, Woodchuck Hepatitis Virus (WHP) posttranscriptional regulatory element (WPRE) (SEQ ID NO: 72) to increase the expression of a transgene. Other posttranscriptional processing elements such as post-transcriptional element from the thymidine kinase gene of herpes simplex virus, or hepatitis B virus (HBV) can be used. The expression cassettes can include a poly-adenylation sequence known in the art or a variation thereof, such as a naturally occurring isolated from bovine BGHpA or a virus SV40 pA (e.g., SEQ ID NO: 10), or synthetic. Some expression cassettes can also include SV40 late polyA signal upstream enhancer (USE) sequence. The USE can be used in combination with SV40 pA or heterologous poly-A signal.

The time for harvesting and collecting DNA vectors described herein from the cells can be selected and optimized to achieve a high-yield production of the DNA vectors. For example, the harvest time can be selected in view of cell viability, cell morphology, cell growth, and the like. Usually, cells can be harvested after sufficient time after baculoviral infection to produce DNA-vectors (e.g., TTX-vectors) but before a majority of the cells start to die because of the viral toxicity. The DNA-vectors can be isolated, for example, using plasmid purification kits such as Qiagen Endo-Free™ Plasmid kits. Other methods developed for plasmid isolation can also be adapted for DNA-vectors. Generally, any nucleic acid purification method known in the art can be adopted.

### Regulatory Sequences and Effectors

In embodiments, the ceDNA vector comprises a second nucleotide sequence (e.g. a regulatory sequence) in addition to the one or more nucleotide sequences encoding a therapeutic protein. In embodiments the gene regulatory sequence is operably linked to the nucleotide sequence encoding the therapeutic protein. In embodiments, the regulatory sequence is suitable for controlling the expression of the therapeutic protein in a host cell. In embodiments, the regulatory sequence includes a suitable promoter sequence, being able to direct transcription of a gene operably linked to the promoter sequence, such as a nucleotide sequence encoding a therapeutic protein of the present disclosure. In embodiments, the second nucleotide sequence includes an intron sequence linked to the 5′ terminus of the nucleotide sequence encoding the therapeutic protein. In embodiments, an enhancer sequence is provided upstream of the promoter to increase the efficacy of the promoter. In embodiments, the regulatory sequence includes an enhancer and a promoter, wherein the second nucleotide sequence includes an intron sequence upstream of the nucleotide sequence encoding a therapeutic protein, wherein the intron includes one or more nuclease cleavage site(s), and wherein the promoter is operably linked to the nucleotide sequence encoding the nuclease. In embodiments, the regulatory sequence used is native to the coding sequence in the vector.

Promoters: Suitable promoters, including those described above, can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 18 (Miyagishi et al., Nature Biotechnology 20, 497-500 (2002)), an enhanced U6 promoter (e.g., Xia et al., Nucleic Acids Res. 2003 Sep. 1; 31 (17)), a human H1 promoter (H1) (e.g., SEQ ID NO: 19), a CAG promoter, a human alpha 1-antitrypsin (HAAT) promoter (e.g., SEQ ID NO: 135), and the like. In embodiments, these promoters are altered at their downstream intron containing end to include one or more nuclease cleavage sites. In embodiments, the DNA containing the nuclease cleavage site(s) is foreign to the promoter DNA.

A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals A promoter may regulate the expression of a gene component constitutively, or differentially with respect to the cell, tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter, as well as the promoters listed below. Such promoters and/or enhancers can be used for expression of any gene of interest, e.g., the gene editing molecules, donor sequence, therapeutic proteins etc.). For example, the vector may comprise a promoter that is operably linked to the nucleic acid sequence encoding a therapeutic protein. The promoter operably linked to the therapeutic protein coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metallothionein. The promoter may also be a tissue specific promoter, such as a liver specific promoter, such as human alpha 1-antitypsin (HAAT), natural or synthetic. In one embodiment, delivery to the liver can be achieved using endogenous ApoE specific targeting of the composition comprising a ceDNA vector to hepatocytes via the low-density lipoprotein (LDL) receptor present on the surface of the hepatocyte.

In one embodiment, the promoter used is the native promoter of the gene encoding the therapeutic protein. The promoters and other regulatory sequences for the respective genes encoding the therapeutic proteins are known and have been characterized. The promoter region used may further include one or more additional regulatory sequences (e.g., native), e.g., enhancers.

Non-limiting examples of suitable promoters for use in accordance with the present invention include the CAG promoter of, for example (SEQ ID NO: 3), the HAAT promoter (SEQ ID NO: 135), the human EF1-α promoter (SEQ ID NO: 6) or a fragment of the EF1-α promoter (SEQ ID NO: 66) and the rat EF1-α promoter (SEQ ID NO: 310).

Enhancers: In some embodiments, a ceDNA expressing an inflammasome antagonist (e.g., inhibitor of one or more of NLRP3 and/or AIM2 inflammasome pathway, or a caspase 1 inhibitor) comprises one or more enhancers. In some embodiments, an enhancer sequence is located 5′ of the promoter sequence. In some embodiments, the enhancer sequence is located 3′ of the promoter sequence. Exemplary enhancers are listed in Table 1 herein.

TABLE 1
Exemplary Enhancer Sequences

| | | Tissue | | |
|---|---|---|---|---|
| | | Speci- | CG | |
| Description | Length | ficity | Content | Sequence |
| cytomegalo- | 518 | Consti- | 22 | TCAATATTGGCCATTAGCCATATTATTCATTGGTTATATAGCATAAATCAATATTGG |
| virus | | tutive | | CTATTGGCCATTGCATACGTTGTATCTATATCATAATATGTACATTTATATTGGCTCA |
| enhancer | | | | TGTCCAATATGACCGCCATGTTGGCATTGATTATTGACTAGTTATTAATAGTAATCA |
| | | | | ATTACGGGGTCATTAGTTCATAGCCCATATATGGAGTTCCGCGTTACATAACTTAC |
| | | | | GGTAAATGGCCCGCCTGGCTGACCGCCCAACGACCCCCGCCCATTGACGTCAATAA |
| | | | | TGACGTATGTTCCCATAGTAACGCCAATAGGGACTTTCCATTGACGTCAATGGGTG |
| | | | | GAGTATTTACGGTAAACTGCCCACTTGGCAGTACATCAAGTGTATCATATGCCAAG |
| | | | | TCCGCCCCCTATTGACGTCAATGACGGTAAATGGCCCGCCTGGCATTATGCCCAGT |
| | | | | ACATGACCTTACGGGACTTTCCTACTTGGCAGTACATCTACGTATTAGTCATCGCTA |
| | | | | TTACCATGG |
| Human | 777 | Liver | 13 | AGGCTCAGAGGCACACAGGAGTTTCTGGGCTCACCCTGCCCCCTTCCAACCCCTCA |
| apolipoprotein | | | | GTTCCCATCCTCCAGCAGCTGTTTGTGTGCTGCCTCTGAAGTCCACACTGAACAAAC |
| E/C-I liver | | | | TTCAGCCTACTCATGTCCCTAAAATGGGCAAACATTGCAAGCAGCAAACAGCAAAC |
| specific | | | | ACACAGCCCTCCCTGCCTGCTGACCTTGGAGCTGGGGCAGAGGTCAGAGACCTCT |
| enhancer | | | | CTGGGCCCATGCCACCTCCAACATCCACTCGACCCCTTGGAATTTCGGTGGAGAGG |
| | | | | AGCAGAGGTTGTCCTGGCGTGGTTTAGGTAGTGTGAGAGGGTCCGGGTTCAAAAC |
| | | | | CACTTGCTGGGTGGGGAGTCGTCAGTAAGTGGCTATGCCCCGACCCCGAAGCCTG |
| | | | | TTTCCCCATCTGTACAATGGAAATGATAAAGACGCCCATCTGATAGGGTTTTTGTG |
| | | | | GCAAATAAACATTTGGTTTTTTTGTTTTGTTTTGTTTTGTTTTTTGAGATGGAGGTTT |
| | | | | GCTCTGTCGCCCAGGCTGGAGTGCAGTGACACAATCTCATCTCACCACAACCTTCC |
| | | | | CCTGCCTCAGCCTCCCAAGTAGCTGGGATTACAAGCATGTGCCACCACACCTGGCT |
| | | | | AATTTTCTATTTTTAGTAGAGACGGGTTTCTCCATGTTGGTCAGCCTCAGCCTCCCA |
| | | | | AGTAACTGGGATTACAGGCCTGTGCCACCACACCCGGCTAATTTTTTCTATTTTTGA |
| | | | | CAGGGACGGGGTTTCACCATGTTGGTCAGGCTGGTCTAGAGGTACCG |
| CpG-free | 427 | Consti- | 0 | GAGTCAATGGGAAAAACCCATTGGAGCCAAGTACACTGACTCAATAGGGACTTTC |
| Murine CMV | | tutive | | CATTGGGTTTTGCCCAGTACATAAGGTCAATAGGGGGTGAGTCAACAGGAAAGTC |
| enhancer | | | | CCATTGGAGCCAAGTACATTGAGTCAATAGGGACTTTCCAATGGGTTTTGCCCAGT |
| | | | | ACATAAGGTCAATGGGAGGTAAGCCAATGGGTTTTTCCCATTACTGACATGTATAC |
| | | | | TGAGTCATTAGGGACTTTCCAATGGGTTTTGCCCAGTACATAAGGTCAATAGGGGT |
| | | | | GAATCAACAGGAAAGTCCCATTGGAGCCAAGTACACTGAGTCAATAGGGACTTTC |
| | | | | CATTGGGTTTTGCCCAGTACAAAAGGTCAATAGGGGGTGAGTCAATGGGTTTTTCC |
| | | | | CATTATTGGCACATACATAAGGTCAATAGGGGTGACTA |
| HS-CRM8 | 83 | Liver | 4 | CGGGGGAGGCTGCTGGTGAATATTAACCAAGGTCACCCCAGTTATCGGAGGAGCA |
| SERP | | | | AACAGGGGCTAAGTCCACACGCGTGGTA |
| enhancer | | | | |
| Human | 777 | Liver | 12 | AGGCTCAGAGGCACACAGGAGTTTCTGGGCTCACCCTGCCCCCTTCCAACCCCTCA |
| apolipoprotein | | | | GTTCCCATCCTCCAGCAGCTGTTTGTGTGCTGCCTCTGAAGTCCACACTGAACAAAC |
| E/C-I liver | | | | TTCAGCCTACTCATGTCCCTAAAATGGGCAAACATTGCAAGCAGCAAACAGCAAAC |
| specific | | | | ACACAGCCCTCCCTGCCTGCTGACCTTGGAGCTGGGGCAGAGGTCAGAGACCTCT |
| enhancer | | | | CTGGGCCCATGCCACCTCCAACATCCACTCGACCCCTTGGAATTTCGGTGGAGAGG |
| | | | | AGCAGAGGTTGTCCTGGCGTGGTTTAGGTAGTGTGAGAGGGTCCGGGTTCAAAAC |
| | | | | CACTTGCTGGGTGGGGAGTCGTCAGTAAGTGGCTATGCCCCGACCCCGAAGCCTG |
| | | | | TTTCCCCATCTGTACAATGGAAATGATAAAGACGCCCATCTGATAGGGTTTTTGTG |
| | | | | GCAAATAAACATTTGGTTTTTTTGTTTTGTTTTGTTTTGTTTTTTGAGATGGAGGTTT |
| | | | | GCTCTGTCGCCCAGGCTGGAGTGCAGTGACACAATCTCATCTCACCACAACCTTCC |
| | | | | CCTGCCTCAGCCTCCCAAGTAGCTGGGATTACAAGCATGTGCCACCACACCTGGCT |
| | | | | AATTTTCTATTTTTAGTAGAGACGGGTTTCTCCATGTTGGTCAGCCTCAGCCTCCCA |
| | | | | AGTAACTGGGATTACAGGCCTGTGCCACCACACCCGGCTAATTTTTTCTATTTTTGA |
| | | | | CAGGGACGGGGTTTCACCATGTTGGTCAGGCTGGTCTAGAGGTACTG |
| 34 bp APOe/c- | 66 | Liver | 1 | GTTTGCTGCTTGCAATGTTTGCCCATTTTAGGGTGGACACAGGACGCTGTGGTTTC |
| 1 Enhancer | | | | TGAGCCAGGG |
| and 32 bp AAT | | | | |
| X-region | | | | |
| Insulting | 212 | Liver | 4 | GGAGGGGTGGAGTCGTGACCCCTAAAATGGGCAAACATTGCAAGCAGCAAACAG |
| sequence and | | | | CAAACACACAGCCCTCCCTGCCTGCTGACCTTGGAGCTGGGGCAGAGGTCAGAGA |
| hAPO-HCR | | | | CCTCTCTGGGCCCATGCCACCTCCAACATCCACTCGACCCCTTGGAATTTCGGTGGA |
| Enhancer | | | | GAGGAGCAGAGGTTGTCCTGGCGTGGTTTAGGTAGTGTGAGAGGGG |
| hAPO-HCR | 330 | Liver | 4 | AGGCTCAGAGGCACACAGGAGTTTCTGGGCTCACCCTGCCCCCTTCCAACCCCTCA |
| Enhancer | | | | GTTCCCATCCTCCAGCAGCTGTTTGTGTGCTGCCTCTGAAGTCCACACTGAACAAAC |
| derived from | | | | TTCAGCCTACTCATGTCCCTAAAATGGGCAAACATTGCAAGCAGCAAACAGCAAAC |
| SPK9001 | | | | ACACAGCCCTCCCTGCCTGCTGACCTTGGAGCTGGGGCAGAGGTCAGAGACCTCT |
| | | | | CTGGGCCCATGCCACCTCCAACATCCACTCGACCCCTTGGAATTTCGGTGGAGAGG |
| | | | | AGCAGAGGTTGTCCTGGCGTGGTTTAGGTAGTGTGAGAGGGGTACCCGGG |
| hAPO-HCR | 194 | Liver | 3 | CCCTAAAATGGGCAAACATTGCAAGCAGCAAACAGCAAACACACAGCCCTCCCTGC |
| Enhancer | | | | CTGCTGACCTTGGAGCTGGGGCAGAGGTCAGAGACCTCTCTGGGCCCATGCCACC |
| | | | | TCCAACATCCACTCGACCCCTTGGAATTTTTCGGTGGAGAGGAGCAGAGGTTGTCC |
| | | | | TGGCGTGGTTTAGGTAGTGTGAGAGGG |
| SV40 | 240 | Consti- | 0 | GGGCCTGAAATAACCTCTGAAAGAGGAACTTGGTTAGGTACCTTCTGAGGCTGAA |
| Enhancer | | tutive | | AGAACCAGCTGTGGAATGTGTGTCAGTTAGGGTGTGGAAAGTCCCCAGGCTCCCC |
| Invivogen | | | | AGCAGGCAGAAGTATGCAAAGCATGCATCTCAATTAGTCAGCAACCAGGTGTGGA |
| | | | | AAGTCCCCAGGCTCCCCAGCAGGCAGAAGTATGCAAAGCATGCATCTCAATTAGTC |
| | | | | AGCAACCATAGTCCCACTA |
| HS-CRM8 | 73 | Liver | 2 | CGGGGGAGGCTGCTGGTGAATATTAACCAAGGTCACCCCAGTTATCGGAGGAGCA |
| SERP enhancer | | | | AACAGGGGCTAAGTCCAC |
| with all | | | | |
| spacers/ | | | | |
| cutsites | | | | |
| removed | | | | |
| Alpha mic/bik | 100 | Liver | 0 | AGGTTAATTTTTAAAAAGCAGTCAAAAGTCCAAGTGGCCCTTGGCAGCATTTACTC |
| Enhancer | | | | TCTCTGTTTGCTCTGGTTAATAATCTCAGGAGCACAAACATTCC |
| CpG-free | 296 | Consti- | 0 | GTTACATAACTTATGGTAAATGGCCTGCCTGGCTGACTGCCCAATGACCCCTGCCC |
| Human CMV | | tutive | | AATGATGTCAATAATGATGTATGTTCCCATGTAATGCCAATAGGGACTTTCCATTGA |
| Enhancer v2 | | | | TGTCAATGGGTGGAGTATTTATGGTAACTGCCCACTTGGCAGTACATCAAGTGTAT |
| | | | | CATATGCCAAGTATGCCCCCTATTGATGTCAATGATGGTAAATGGCCTGCCTGGCA |
| | | | | TTATGCCCAGTACATGACCTTATGGGACTTTCCTACTTGGCAGTACATCTATGTATT |
| | | | | AGTCATTGCTATTA |
| SV40 | 235 | Consti- | 1 | GGCCTGAAATAACCTCTGAAAGAGGAACTTGGTTAGGTACCTTCTGAGGCGGAAA |
| Enhancer | | tutive | | GAACCAGCTGTGGAATGTGTGTCAGTTAGGGTGTGGAAAGTCCCCAGGCTCCCCA |
| | | | | GCAGGCAGAAGTATGCAAAGCATGCATCTCAATTAGTCAGCAACCAGGTGTGGAA |
| | | | | AGTCCCCAGGCTCCCCAGCAGGCAGAAGTATGCAAAGCATGCATCTCAATTAGTCA |
| | | | | GCAACCATAGTCCC |

5′ UTR sequences and intron sequences: In some embodiments, a ceDNA vector comprises a 5′ UTR sequence and/or an intron sequence that located 3′ of the 5′ ITR sequence. In some embodiments, the 5′ UTR is located 5′ of the transgene, e.g., sequence encoding an inflammasome antagonist (e.g., inhibitor of one or more of NLRP3 and/or AIM2 inflammasome pathway, or a caspase 1 inhibitor). Exemplary 5′ UTR sequences listed in Table 2A.

TABLE 2A
Exemplary 5′ UTR sequences and intron sequences

| | | | CG | |
|---|---|---|---|---|
| Description | Length | Reference | Content | Sequence |
| synthetic 5′ UTR | 1127 | | 137 | GGAGTCGCTGCGACGCTGCCTTCGCCCCGTGCCCCGCTCCGCCGCCGCCTC |
| element composed | | | | GCGCCGCCCGCCCCGGCTCTGACTGACCGCGTTACTCCCACAGGTGAGCGG |
| of chicken B-actin | | | | GCGGGACGGCCCTTCTCCTCCGGGCTGTAATTAGCGCTTGGTTTAATGACG |
| 5′UTR/Intron and | | | | GCTTGTTTCTTTTCTGTGGCTGCGTGAAAGCCTTGAGGGGCTCCGGGAGGG |
| rabbit B-globin | | | | CCCTTTGTGCGGGGGGGAGCGGCTCGGGGGGTGCGTGCGTGTGTGTGTGC |
| intron and 1st exon | | | | GTGGGGAGCGCCGCGTGCGGCCCGCGCTGCCCGGCGGCTGTGAGCGCTGC |
| | | | | GGGCGCGGCGCGGGGCTTTGTGCGCTCCGCAGTGTGCGCGAGGGGAGCG |
| | | | | CGGCCGGGGGCGGTGCCCCGCGGTGCGGGGGGGGCTGCGAGGGGAACA |
| | | | | AAGGCTGCGTGCGGGGTGTGTGCGTGGGGGGGTGAGCAGGGGGTGTGG |
| | | | | GCGCGGCGGTCGGGCTGTAACCCCCCCCTGCACCCCCCTCCCCGAGTTGCT |
| | | | | GAGCACGGCCCGGCTTCGGGTGCGGGGCTCCGTACGGGGCGTGGCGCGG |
| | | | | GGCTCGCCGTGCCGGGCGGGGGGTGGCGGCAGGTGGGGGTGCCGGGCG |
| | | | | GGGCGGGGCCGCCTCGGGCCGGGGAGGGCTCGGGGGAGGGGCGCGGCG |
| | | | | GCCCCCGGAGCGCCGGCGGCTGTCGAGGCGCGGCGAGCCGCAGCCATTGC |
| | | | | CTTTTATGGTAATCGTGCGAGAGGGCGCAGGGACTTCCTTTGTCCCAAATCT |
| | | | | GTGCGGAGCCGAAATCTGGGAGGCGCCGCCGCACCCCCTCTAGCGGGCGC |
| | | | | GGGGCGAAGCGGTGCGGCGCCGGCAGGAAGGAAATGGGCGGGGAGGGC |
| | | | | CTTCGTGCGTCGCCGCGCCGCCGTCCCCTTCTCCCTCTCCAGCCTCGGGGCT |
| | | | | GTCCGCGGGGGGACGGCTGCCTTCGGGGGGGACGGGGCAGGGCGGGGTT |
| | | | | CGGCTTCTGGCGTGTGACCGGCGGCTCTAGAGCCTCTGCTAACCATGTTTTA |
| | | | | GCCTTCTTCTTTTTCCTACAGCTCCTGGGCAACGTGCTGGTTATTGTGCTGTC |
| | | | | TCATCATTTGTCGACAGAATTCCTCGAAGATCCGAAGGGGTTCAAGCTTGG |
| | | | | CATTCCGGTACTGTTGGTAAAGCCA |
| modified SV40 | 93 | | 0 | CTCTAAGGTAAATATAAAATTTTTAAGTGTATAATGTGTTAAACTACTGATT |
| Intron | | | | CTAATTGTTTCTCTCTTTTAGATTCCAACCTTTGGAACTGA |
| 5′ UTR of hAAT just | 54 | | 1 | GCCCTGTCTCCTCAGCTTCAGGCACCACCACTGACCTGGGACAGTGAATCC |
| upstream of ORF (3′ | | | | GGA |
| CGGA may be | | | | |
| spacer/restriction | | | | |
| enzyme cut site, | | | | |
| and was absorbed | | | | |
| into the sequence) | | | | |
| CET promotor set | 173 | | 0 | CTGCCTTCTCCCTCCTGTGAGTTTGGTAAGTCACTGACTGTCTATGCCTGGG |
| synthetic intron | | | | AAAGGGTGGGCAGGAGATGGGGCAGTGCAGGAAAAGTGGCACTATGAAC |
| | | | | CCTGCAGCCCTAGACAATTGTACTAACCTTCTTCTCTTTCCTCTCCTGACAGG |
| | | | | TTGGTGTACAGTAGCTTCC |
| Minute Virus Mice | 91 | | 0 | AAGAGGTAAGGGTTTAAGGGATGGTTGGTTGGTGGGGTATTAATGTTTAA |
| (MVM) Intron | | | | TTACCTGGAGCACCTGCCTGAAATCACTTTTTTTCAGGTTG |
| 5′ UTR of hAAT | 54 | | 0 | GCCCTGTCTCCTCAGCTTCAGGCACCACCACTGACCTGGGACAGTGAATAAT |
| | | | | TA |
| 5′ UTR of hAAT | 147 | | 1 | GCCCTGTCTCCTCAGCTTCAGGCACCACCACTGACCTGGGACAGTGAATCC |
| combined with | | | | GGACTCTAAGGTAAATATAAAATTTTTAAGTGTATAATGTGTTAAACTACTG |
| modSV40 intron | | | | ATTCTAATTGTTTCTCTCTTTTAGATTCCAACCTTTGGAACTGA |
| 5′ UTR of hAAT (3′ | 147 | | 0 | GCCCTGTCTCCTCAGCTTCAGGCACCACCACTGACCTGGGACAGTGAATAAT |
| TAATTA may be | | | | TACTCTAAGGTAAATATAAAATTTTTAAGTGTATAATGTGTTAAACTACTGA |
| spacer/restriction | | | | TTCTAATTGTTTCTCTCTTTTAGATTCCAACCTTTGGAACTGA |
| enzyme cut site, | | | | |
| and was absorbed | | | | |
| into the sequence) | | | | |
| combined with | | | | |
| modSV40 intron | | | | |
| 42 bp of 5′ UTR of | 48 | https:// | 1 | TCCTCAGCTTCAGGCACCACCACTGACCTGGGACAGTGAATCGCCACC |
| AAT derived from | | www.ncbi. | | |
| BMN270-includes | | nlm.nih.gov/ | | |
| Kozak | | pubmed/ | | |
| | | 29292164 | | |
| Intron/Enhancer | 128 | US2017/ | 6 | GCTAGCAGGTAAGTGCCGTGTGTGGTTCCCGCGGGCCTGGCCTCTTTACGG |
| from EF1-α | | 0216408 | | GTTATGGCCCTTGCGTGCCTTGAATTACTGACACTGACATCCACTTTTTCTTT |
| | | | | TTCTCCACAGGTTTAAACGCCACC |
| Synthetic SBR intron | 98 | WO2017074526 | 2 | AAGAGGTAAGGGTTTAAGTTATCGTTAGTTCGTGCACCATTAATGTTTAATT |
| derived from | | | | ACCTGGAGCACCTGCCTGAAATCATTTTTTTTTCAGGTTGGCTAGT |
| Sangamo CRMSBS2- | | | | |
| Intron3--includes | | | | |
| kozak | | | | |
| Endogenous hFVIII | 172 | NG_011403.1 | 0 | GCTTAGTGCTGAGCACATCCAGTGGGTAAAGTTCCTTAAAATGCTCTGCAA |
| 5′ UTR | | | | AGAAATTGGGACTTTTCATTAAATCAGAAATTTTACTTTTTTCCCCTCCTGGG |
| | | | | AGCTAAAGATATTTTAGAGAAGAATTAACCTTTTGCTTCTCCAGTTGAACAT |
| | | | | TTGTAGCAATAAGTCA |
| hAAT 5′ UTR | 160 | http:// | 1 | GCCCTGTCTCCTCAGCTTCAGGCACCACCACTGACCTGGGACAGTGAATCC |
| modSV40 + kozak | | www.blood | | GGACTCTAAGGTAAATATAAAATTTTTAAGTGTATAATGTGTTAAACTACTG |
| | | journal.org/ | | ATTCTAATTGTTTCTCTCTTTTAGATTCCAACCTTTGGAACTGAATTCTAGAC |
| | | content/ | | CACC |
| | | early/2005/ | | |
| | | 12/01/blood- | | |
| | | 2005- | | |
| | | 10- | | |
| | | 4035?sso- | | |
| | | checked= | | |
| | | true | | |
| hFIX 5′ UTR and | 29 | US201603 | 0 | ACCACTTTCACAATCTGCTAGCAAAGGTT |
| Kozak | | 75110 | | |
| Chimeric Intron | 133 | U47119.2 | 2 | GTAAGTATCAAGGTTACAAGACAGGTTTAAGGAGACCAATAGAAACTGGG |
| | | | | CTTGTCGAGACAGAGAAGACTCTTGCGTTTCTGATAGGCACCTATTGGTCTT |
| | | | | ACTGACATCCACTTTGCCTTTCTCTCCACAG |
| Large fragment of | 341 | | 9 | TGGGCAGGAACTGGGCACTGTGCCCAGGGCATGCACTGCCTCCACGCAGC |
| Human Alpha-1 | | | | AACCCTCAGAGTCCTGAGCTGAACCAAGAAGGAGGAGGGGGTCGGGCCTC |
| Antitrypsin (AAT) 5′ | | | | CGAGGAAGGCCTAGCCGCTGCTGCTGCCAGGAATTCCAGGTTGGAGGGGC |
| UTR | | | | GGCAACCTCCTGCCAGCCTTCAGGCCACTCTCCTGTGCCTGCCAGAAGAGA |
| | | | | CAGAGCTTGAGGAGAGCTTGAGGAGAGCAGGAAAGCCTCCCCCGTTGCCC |
| | | | | CTCTGGATCCACTGCTTAAATACGGACGAGGACAGGGCCCTGTCTCCTCAG |
| | | | | CTTCAGGCACCACCACTGACCTGGGACAGTGAATCGACA |
| 5pUTR | 316 | US9644216 | 6 | TCTAGAGAAGCTTTATTGCGGTAGTTTATCACAGTTAAATTGCTAACGCAGT |
| | | | | CAGTGCTTCTGACACAACAGTCTCGAACTTAAGCTGCAGTGACTCTCTTAAG |
| | | | | GTAGCCTTGCAGAAGTTGGTCGTGAGGCACTGGGCAGGTAAGTATCAAGG |
| | | | | TTACAAGACAGGTTTAAGGAGACCAATAGAAACTGGGCTTGTCGAGACAG |
| | | | | AGAAGACTCTTGCGTTTCTGATAGGCACCTATTGGTCTTACTGACATCCACT |
| | | | | TTGCCTTTCTCTCCACAGGTGTCCACTCCCAGTTCAATTACAGCTCTTAAGGC |
| | | | | CCTGCAG |
| Human cDNA | 76 | NM_000443 | 8 | CAAAGTCCAGGCCCCTCTGCTGCAGCGCCCGCGCGTCCAGAGGCCCTGCCA |
| ABCB4 5pUTR | | | | GACACGCGCGAGGTTCGAGGCTGAG |
| (Variant A, | | | | |
| predominant | | | | |
| Isoform) | | | | |
| Human cDNA | 127 | NM_003742 | 2 | AGAATGATGAAAACCGAGGTTGGAAAAGGTTGTGAAACCTTTTAACTCTCC |
| ABCB11 5pUTR | | | | ACAGTGGAGTCCATTATTTCCTCTGGCTTCCTCAAATTCATATTCACAGGGTC |
| | | | | GTTGGCTGTGGGTTGCAATTACC |
| Human G6Pase | 80 | NM_000151.3 | 0 | ATAGCAGAGCAATCACCACCAAGCCTGGAATAACTGCAAGGGCTCTGCTGA |
| 5pUTR | | | | CATCTTCCTGAGGTGCCAAGGAAATGAGG |
| MCK 5pUTR derived | 208 | https:// | 8 | GGGTCACCACCACCTCCACAGCACAGACAGACACTCAGGAGCCAGCCAGCC |
| from | | patentimages. | | AGGTAAGTTTAGTCTTTTTGTCTTTTATTTCAGGTCCCGGATCCGGTGGTGG |
| rAAVi rh 74. MCK | | storage. | | TGCAAATCAAAGAACTGCTCCTCAGTGGATGTTGCCTTTACTTCTAGGCCTG |
| GALGT2. Contains | | googleapis. | | TACGGAAGTGTTACTTCTGCTCTAAAAGCTGCGGAATTGTACCCGCGGCCG |
| 53 bp of | | com/4f/8a/d6/ | | CG |
| endogenous mouse | | b915c650f5eeb5/ | | |
| MCK Exon1 | | WO2017049031A1. | | |
| (untranslated), | | pdf | | |
| SV40 late 16S/19S | | | | |
| splice signals, | | | | |
| 5pUTR derived from | | | | |
| plasmid pCMVB. | | | | |
| CpG Free 5′ UTR | 159 | | 0 | AAGCTTCTGCCTTCTCCCTCCTGTGAGTTTGGTAAGTCACTGACTGTCTATGC |
| synthetic (SI 126) | | | | CTGGGAAAGGGTGGGCAGGAGATGGGGCAGTGCAGGAAAAGTGGCACTA |
| Intron | | | | TGAACCCTGCAGCCCTAGACAATTGTACTAACCTTCTTCTCTTTCCTCTCCTG |
| | | | | ACAG |
| 5′ UTR of Human | 36 | (NM_000101.4) | 5 | CGCGCCTAGCAGTGTCCCAGCCGGGTTCGTGTCGCC |
| Cytochrome b-245 | | | | |
| alpha chain (CYBA) | | | | |
| gene | | | | |
| 5′ UTR of Human | 141 | (NM_001330575.1) | 14 | ACGCCGCCTGGGTCCCAGTCCCCGTCCCATCCCCCGGCGGCCTAGGCAGCG |
| 2,4-dienoyl-CoA | | | | TTTCCAGCCCCGAGAACTTTGTTCTTTTTGTCCCGCCCCCTGCGCCCAACCGC |
| red uctase 1 (DECR1) | | | | CTGCGCCGCCTTCCGGCCCGAGTTCTGGAGACTCAAC |
| gene | | | | |
| 5′ UTR of Human | 110 | (NM_001301008.1) | 4 | GTTGGATGAAACCTTCCTCCTACTGCACAGCCCGCCCCCCTACAGCCCCGGT |
| glia maturation | | | | CCCCACGCCTAGAAGACAGCGGAACTAAGAAAAGAAGAGGCCTGTGGACA |
| factor gamma | | | | GAACAATC |
| (GMFG) gene | | | | |
| 5′ UTR of Human | 164 | (NM_001145264.1) | 13 | GGTGGGGCGGGGTTGAGTCGGAACCACAATAGCCAGGCGAAGAAACTAC |
| late | | | | AACTCCCAGGGCGTCCCGGAGCAGGCCAACGGGACTACGGGAAGCAGCG |
| endosomal/lysosomal | | | | GGCAGCGGCCCGCGGGAGGCACCTCGGAGATCTGGGTGCAAAAGCCCAG |
| adaptor, MAPK | | | | GGTTAGGAACCGTAGGC |
| and MTOR activator | | | | |
| 2 (LAMTOR2) | | | | |
| 5′ UTR of Human | 127 | (NM_002475.4) | 8 | GGCCACCGGAATTAACCCTTCAGGGCTGGGGGCCGCGCTATGCCCCGCCCC |
| myosin light chain | | | | CTCCCCAGCCCCAGACACGGACCCCGCAGGAGATGGGTGCCCCCATCCGCA |
| 6B (MYL6B) | | | | CACTGTCCTTTGGCCACCGGACATC |
| Large fragment of | 341 | | 9 | TGGGCAGGAACTGGGCACTGTGCCCAGGGCATGCACTGCCTCCACGCAGC |
| Human Alpha-1 | | | | AACCCTCAGAGTCCTGAGCTGAACCAAGAAGGAGGAGGGGGTCGGGCCTC |
| Antitrypsin (AAT) 5′ | | | | CGAGGAAGGCCTAGCCGCTGCTGCTGCCAGGAATTCCAGGTTGGAGGGGC |
| UTR | | | | GGCAACCTCCTGCCAGCCTTCAGGCCACTCTCCTGTGCCTGCCAGAAGAGA |
| | | | | CAGAGCTTGAGGAGAGCTTGAGGAGAGCAGGAAAGCCTCCCCCGTTGCCC |
| | | | | CTCTGGATTCACTGCTTAAATACGGACGAGGACAGGGCCCTGTCTCCTCAG |
| | | | | CTTCAGGCACCACCACTGACCTGGGACAGTGAATCGACA |

3′ UTR Sequences: In some embodiments, a ceDNA vector comprises a 3′ UTR sequence that located 5′ of the 3′ ITR sequence. In some embodiments, the 3′ UTR is located 3′ of the transgene, e.g., sequence encoding an inflammasome antagonist (e.g., inhibitor of one or more of NLRP3 and/or AIM2 inflammasome pathway, or a caspase 1 inhibitor). Exemplary 3′ UTR sequences listed in Table 2B.

TABLE 2B
Exemplary 3′ UTR sequences and intron sequences

| | | | CG | |
|---|---|---|---|---|
| Description | Length | Reference | Content | Sequence |
| WHP | 581 | | 20 | GAGCATCTTACCGCCATTTATTCCCATATTTGTTCTGTTTTTCTTGATTTGGGTATACAT |
| Posttranscriptional | | | | TTAAATGTTAATAAAACAAAATGGTGGGGCAATCATTTACATTTTTAGGGATATGTAA |
| Response | | | | TTACTAGTTCAGGTGTATTGCCACAAGACAAACATGTTAAGAAACTTTCCCGTTATTTA |
| Element | | | | CGCTCTGTTCCTGTTAATCAACCTCTGGATTACAAAATTTGTGAAAGATTGACTGATAT |
| | | | | TCTTAACTATGTTGCTCCTTTTACGCTGTGTGGATATGCTGCTTTATAGCCTCTGTATCT |
| | | | | AGCTATTGCTTCCCGTACGGCTTTCGTTTTCTCCTCCTTGTATAAATCCTGGTTGCTGTC |
| | | | | TCTTTTAGAGGAGTTGTGGCCCGTTGTCCGTCAACGTGGCGTGGTGTGCTCTGTGTTT |
| | | | | GCTGACGCAACCCCCACTGGCTGGGGCATTGCCACCACCTGTCAACTCCTTTCTGGGA |
| | | | | CTTTCGCTTTCCCCCTCCCGATCGCCACGGCAGAACTCATCGCCGCCTGCCTTGCCCGC |
| | | | | TGCTGGACAGGGGCTAGGTTGCTGGGCACTGATAATTCCGTGGTGTTGTC |
| Triplet repeat | 77 | | 1 | TCCATAAAGTAGGAAACACTACACGATTCCATAAAGTAGGAAACACTACATCACTCCA |
| of mir-142 | | | | TAAAGTAGGAAACACTACA |
| binding site | | | | |
| hFIX 3′ UTR | 88 | US2016/ | 0 | TGAAAGATGGATTTCCAAGGTTAATTCATTGGAATTGAAAATTAACAGAGATCTAGA |
| and polyA | | 0375110 | | GCTGAATTCCTGCAGCCAGGGGGATCAGCCT |
| spacer derived | | | | |
| from SPK9001 | | | | |
| Human | 395 | | 1 | TAAAATACAGCATAGCAAAACTTTAACCTCCAAATCAAGCCTCTACTTGAATCCTTTTC |
| hemoglobin | | | | TGAGGGATGAATAAGGCATAGGCATCAGGGGCTGTTGCCAATGTGCATTAGCTGTTT |
| beta (HBB) | | | | GCAGCCTCACCTTCTTTCATGGAGTTTAAGATATAGTGTATTTTCCCAAGGTTTGAACT |
| 3pUTR | | | | AGCTCTTCATTTCTTTATGTTTTAAATGCACTGACCTCCCACATTCCCIIIITAGTAAAA |
| | | | | TATTCAGAAATAATTTAAATACATCATTGCAATGAAAATAAATGTTTTTTATTAGGCAG |
| | | | | AATCCAGATGCTCAAGGCCCTTCATAATATCCCCCAGTTTAGTAGTTGGACTTAGGGA |
| | | | | ACAAAGGAACCTTTAATAGAAATTGGACAGCAAGAAAGCGAGC |
| Interferon | 800 | | 0 | AGTCAATATGTTCACCCCAAAAAAGCTGTTTGTTAACTTGCCAACCTCATTCTAAAATG |
| Beta S/MAR | | | | TATATAGAAGCCCAAAAGACAATAACAAAAATATTCTTGTAGAACAAAATGGGAAAG |
| (Scaffold/matrix- | | | | AATGTTCCACTAAATATCAAGATTTAGAGCAAAGCATGAGATGTGTGGGGATAGACA |
| associated | | | | GTGAGGCTGATAAAATAGAGTAGAGCTCAGAAACAGACCCATTGATATATGTAAGTG |
| Region) | | | | ACCTATGAAAAAAATATGGCATTTTACAATGGGAAAATGATGGTCTTTTTCTTTTTTAG |
| | | | | AAAAACAGGGAAATATATTTATATGTAAAAAATAAAAGGGAACCCATATGTCATACC |
| | | | | ATACACACAAAAAAATTCCAGTGAATTATAAGTCTAAATGGAGAAGGCAAAACTTTA |
| | | | | AATCTTTTAGAAAATAATATAGAAGCATGCCATCAAGACTTCAGTGTAGAGAAAAATT |
| | | | | TCTTATGACTCAAAGTCCTAACCACAAAGAAAAGATTGTTAATTAGATTGCATGAATA |
| | | | | TTAAGACTTATTTTTAAAATTAAAAAACCATTAAGAAAAGTCAGGCCATAGAATGACA |
| | | | | GAAAATATTTGCAACACCCCAGTAAAGAGAATTGTAATATGCAGATTATAAAAAGAA |
| | | | | GTCTTACAAATCAGTAAAAAATAAAACTAGACAAAAATTTGAACAGATGAAAGAGAA |
| | | | | ACTCTAAATAATCATTACACATGAGAAACTCAATCTCAGAAATCAGAGAACTATCATT |
| | | | | GCATATACACTAAATTAGAGAAATATTAAAAGGCTAAGTAACATCTGTGGC |
| Beta-Globulin | 407 | | 0 | AATTATCTCTAAGGCATGTGAACTGGCTGTCTTGGTTTTCATCTGTACTTCATCTGCTA |
| MAR (Matrix- | | | | CCTCTGTGACCTGAAACATATTTATAATTCCATTAAGCTGTGCATATGATAGATTTATC |
| associated | | | | ATATGTATTTTCCTTAAAGGATTTTTGTAAGAACTAATTGAATTGATACCTGTAAAGTC |
| region) | | | | TTTATCACACTACCCAATAAATAATAAATCTCTTTGTTCAGCTCTCTGTTTCTATAAATA |
| | | | | TGTACCAGTTTTATTGTTTTTAGTGGTAGTGATTTTATTCTCTTTCTATATATATACACA |
| | | | | CACATGTGTGCATTCATAAATATATACAATTTTTATGAATAAAAAATTATTAGCAATCA |
| | | | | ATATTGAAAACCACTGATTTTTGTTTATGTGAGCAAACAGCAGATTAAAAG |
| Human | 186 | | 1 | CATCACATTTAAAAGCATCTCAGCCTACCATGAGAATAAGAGAAAGAAAATGAAGAT |
| Albumin 3′ | | | | CAAAAGCTTATTCATCTGTTTTTCTTTTTCGTTGGTGTAAAGCCAACACCCTGTCTAAA |
| UTR Sequence | | | | AAACATAAATTTCTTTAATCATTTTGCCTCTTTTCTCTGTGCTTCAATTAATAAAAAATG |
| | | | | GAAAGAATCT |
| CpG | 395 | | 0 | TAAAATACAGCATAGCAAAACTTTAACCTCCAAATCAAGCCTCTACTTGAATCCTTTTC |
| minimized | | | | TGAGGGATGAATAAGGCATAGGCATCAGGGGCTGTTGCCAATGTGCATTAGCTGTTT |
| HBB 3pUTR | | | | GCAGCCTCACCTTCTTTCATGGAGTTTAAGATATAGTGTATTTTCCCAAGGTTTGAACT |
| | | | | AGCTCTTCATTTCTTTATGTTTTAAATGCACTGACCTCCCACATTCCCTTTTTAGTAAAA |
| | | | | TATTCAGAAATAATTTAAATACATCATTGCAATGAAAATAAATGTTTTTTATTAGGCAG |
| | | | | AATCCAGATGCTCAAGGCCCTTCATAATATCCCCCAGTTTAGTAGTTGGACTTAGGGA |
| | | | | ACAAAGGAACCTTTAATAGAAATTGGACAGCAAGAAAGCCAGC |
| WHP | 580 | | 20 | GAGCATCTTACCGCCATTTATTCCCATATTTGTTCTGTTTTTCTTGATTTGGGTATACAT |
| Posttranscriptional | | | | TTAAATGTTAATAAAACAAAATGGTGGGGCAATCATTTACATTTTTAGGGATATGTAA |
| Response | | | | TTACTAGTTCAGGTGTATTGCCACAAGACAAACATGTTAAGAAACTTTCCCGTTATTTA |
| Element. | | | | CGCTCTGTTCCTGTTAATCAACCTCTGGATTACAAAATTTGTGAAAGATTGACTGATAT |
| Missing 3′ | | | | TCTTAACTATGTTGCTCCTTTTACGCTGTGTGGATATGCTGCTTTATAGCCTCTGTATCT |
| Cytosine. | | | | AGCTATTGCTTCCCGTACGGCTTTCGTTTTCTCCTCCTTGTATAAATCCTGGTTGCTGTC |
| | | | | TCTTTTAGAGGAGTTGTGGCCCGTTGTCCGTCAACGTGGCGTGGTGTGCTCTGTGTTT |
| | | | | GCTGACGCAACCCCCACTGGCTGGGGCATTGCCACCACCTGTCAACTCCTTTCTGGGA |
| | | | | CTTTCGCTTTCCCCCTCCCGATCGCCACGGCAGAACTCATCGCCGCCTGCCTTGCCCGC |
| | | | | TGCTGGACAGGGGCTAGGTTGCTGGGCACTGATAATTCCGTGGTGTTGT |
| 3′ UTR of | 64 | (NM_000101.4) | 5 | CCTCGCCCCGGACCTGCCCTCCCGCCAGGTGCACCCACCTGCAATAAATGCAGCGAA |
| Human | | | | GCCGGGA |
| Cytochrome b- | | | | |
| 245 alpha | | | | |
| chain (CYBA) | | | | |
| gene | | | | |
| Shortened | 247 | WPRE | 10 | GATAATCAACCTCTGGATTACAAAATTTGTGAAAGATTGACTGGTATTCTTAACTATGT |
| WPRE3 | | 3 ref | | TGCTCCTTTTACGCTATGTGGATACGCTGCTTTAATGCCTTTGTATCATGCTATTGCTTC |
| sequence with | | https: | | CCGTATGGCTTTCATTTTCTCCTCCTTGTATAAATCCTGGTTAGTTCTTGCCACGGCGG |
| minimal | | ncbi. | | AACTCATCGCCGCCTGCCTTGCCCGCTGCTGGACAGGGGCTCGGCTGTTGGGCACTG |
| gamma and | | nlm.nih. | | ACAATTCCGTGG |
| alpha elements | | gov/pmc/ | | |
| | | articles/ | | |
| | | PMC3975461/ | | |
| Human | 144 | | 1 | AAATACATCATTGCAATGAAAATAAATGTTTTTTATTAGGCAGAATCCAGATGCTCAA |
| hemoglobin | | | | GGCCCTTCATAATATCCCCCAGTTTAGTAGTTGGACTTAGGGAACAAAGGAACCTTTA |
| beta (HBB) | | | | ATAGAAATTGGACAGCAAGAAAGCGAGC |
| 3pUTR | | | | |
| First 62 bp of | 62 | | 1 | GAGCATCTTACCGCCATTTATTCCCATATTTGTTCTGTTTTTCTTGATTTGGGTATACAT |
| WPRE 3pUTR | | | | TT |
| element | | | | |

Polyadenylation Sequences: A sequence encoding a polyadenylation sequence can be included in the ceDNA vector for expression of an inhibitor of the immune response (e.g., the innate immune response) as described herein to stabilize an mRNA expressed from the ceDNA vector, and to aid in nuclear export and translation. In one embodiment, the ceDNA vector does not include a polyadenylation sequence. In other embodiments, the ceDNA vector for expression of an infammasome antagonist includes at least 1, at least 2, at least 3, at least 4, at least 5, at least 10, at least 15, at least 20, at least 25, at least 30, at least 40, least 45, at least 50 or more adenine dinucleotides. In some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include any poly-adenylation sequence known in the art or a variation thereof. In some embodiments, a poly-adenylation (polyA) sequence is selected from any of those listed in Table 3. Other polyA sequences commonly known in the art can also be used, e.g., including but not limited to, naturally occurring sequence isolated from bovine BGHpA (e.g., SEQ ID NO: 9) or a virus SV40 pA (e.g., SEQ ID NO: 10), or a synthetic sequence. Some expression cassettes can also include SV40 late polyA signal upstream enhancer (USE) sequence. In some embodiments, a USE sequence can be used in combination with SV40 pA or heterologous poly-A signal. PolyA sequences are located 3′ of the transgene encoding an infammasome antagonist.

The expression cassettes can also include a post-transcriptional element to increase the expression of a transgene. In some embodiments, Woodchuck Hepatitis Virus (WHP) posttranscriptional regulatory element (WPRE) (e.g., SEQ ID NO: 72) is used to increase the expression of a transgene. Other posttranscriptional processing elements such as the post-transcriptional element from the thymidine kinase gene of herpes simplex virus, or hepatitis B virus (HBV) can be used. Secretory sequences can be linked to the transgenes, e.g., VH-02 and VK-A26 sequences, e.g., SEQ ID NO: 950 and SEQ ID NO: 951.

TABLE 3
Exemplary polyA sequences

| | | | CG | |
|---|---|---|---|---|
| Description | Length | Reference | Content | Sequence |
| bovine growth | 225 | | 3 | TGTGCCTTCTAGTTGCCAGCCATCTGTTGTTTGCCCCTCCCCCGTGCCTTCCTTGAC |
| hormone | | | | CCTGGAAGGTGCCACTCCCACTGTCCTTTCCTAATAAAATGAGGAAATTGCATCG |
| Terminator and | | | | CATTGTCTGAGTAGGTGTCATTCTATTCTGGGGGGTGGGGTGGGGCAGGACAGC |
| poly- | | | | AAGGGGGAGGATTGGGAAGACAATAGCAGGCATGCTGGGGATGCGGTGGGCT |
| adenylation | | | | CTATGGC |
| seqience. | | | | |
| Synthetic polyA | 49 | https://www. | 0 | AATAAAAGATCTTTATTTTCATTAGATCTGTGTGTTGGTTTTTTGTGTG |
| derived from | | ncbi.nlm.nih. | | |
| BMN270 | | gov/pubmed/ | | |
| | | 29292164 | | |
| Synthetic polyA | 54 | US2017/ | 2 | GCGGCCGCAATAAAAGATCAGAGCTCTAGAGATCTGTGTGTTGGTTTTTTGTGT |
| derived from | | 0216408 | | |
| SPK8011 | | | | |
| Synthetic polyA | 74 | WO2017074526 | 2 | GGATCCAATAAAATATCTTTATTTTCATTACATCTGTGTGTTGGTTTTTTGTGTGTT |
| and insulating | | | | TTCCTGTAACGATCGGG |
| sequence | | | | |
| derived from | | | | |
| Sangamo_CRM | | | | |
| SBS2-Intron3 | | | | |
| SV40 Late polyA | 143 | http://www. | 1 | CTCGATGCTTTATTTGTGAAATTTGTGATGCTATTGCTTTATTTGTAACCATTATAA |
| and 3′ | | bloodjournal. | | GCTGCAATAAACAAGTTAACAACAACAATTGCATTCATTTTATGTTTCAGGTTCAG |
| Insulating | | org/content/ | | GGGGAGGTGTGGGAGGTTTTTTAAACTAGT |
| sequence | | early/2005/ | | |
| derived from | | 12/01/blood- | | |
| Nathwani hFIX | | 2005-10- | | |
| | | 4035?sso- | | |
| | | checked=true | | |
| bGH polyA | 228 | US2016/ | 0 | CTACTGTGCCTTCTAGTTGCCAGCCATCTGTTGTTTGCCCCTCCCCCTTGCCTTCCT |
| derived from | | 0375110 | | TGACCCTGGAAGGTGCCACTCCCACTGTCCTTTCCTAATAAAATGAGGAAATTGC |
| SPK9001 | | | | ATCACATTGTCTGAGTAGGTGTCATTCTATTCTGGGGGGTGGGGTGGGGCAGGA |
| | | | | CAGCAAGGGGGAGGATTGGGAAGACAATAGCAGGCATGCTGGGGATGCAGTG |
| | | | | GGCTCTATGG |
| CpGfree SV40 | 222 | | 0 | CAGACATGATAAGATACATTGATGAGTTTGGACAAACCACAACTAGAATGCAGT |
| polyA | | | | GAAAAAAATGCTTTATTTGTGAAATTTGTGATGCTATTGCTTTATTTGTAACCATT |
| | | | | ATAAGCTGCAATAAACAAGTTAACAACAACAATTGCATTCATTTTATGTTTCAGGT |
| | | | | TCAGGGGGAGATGTGGGAGGTTTTTTAAAGCAAGTAAAACCTCTACAAATGTGG |
| | | | | TA |
| SV40 late polyA | 226 | | 0 | CCAGACATGATAAGATACATTGATGAGTTTGGACAAACCACAACTAGAATGCAGT |
| | | | | GAAAAAAATGCTTTATTTGTGAAATTTGTGATGCTATTGCTTTATTTGTAACCATT |
| | | | | ATAAGCTGCAATAAACAAGTTAACAACAACAATTGCATTCATTTTATGTTTCAGGT |
| | | | | TCAGGGGGAGGTGTGGGAGGTTTTTTAAAGCAAGTAAAACCTCTACAAATGTGG |
| | | | | TATGG |
| C60pAC30HSL | 129 | | 0 | GTTAACAAAAAAAAAAAAAAAAAAAAAAAAAAAAAAAAAAAAAAAAAAAAAAA |
| polyA | | | | AAAAAAAAAAAAAAAAATGCATCCCCCCCCCCCCCCCCCCCCCCCCCCCCCCCAAA |
| containing A64 | | | | GGCTCTTTTCAGAGCCACCA |
| polyA sequence | | | | |
| and C30 histone | | | | |
| stem loop | | | | |
| sequence | | | | |
| polyA used in J. | 232 | US9644216 | 4 | GCGGCCGCGGGGATCCAGACATGATAAGATACATTGATGAGTTTGGACAAACCA |
| Chou G6Pase | | | | CAACTAGAATGCAGTGAAAAAAATGCTTTATTTGTGAAATTTGTGATGCTATTGCT |
| constructs | | | | TTATTTGTAACCATTATAAGCTGCAATAAACAAGTTAACAACAACAATTGCATTCA |
| containing a | | | | TTTTATGTTTCAGGTTCAGGGGGAGGTGTGGGAGGTTTTTTAGTCGACCATGCTG |
| SV40 polyA | | | | GGGAGAGATCT |
| SV40 | 135 | | 0 | GATCCAGACATGATAAGATACATTGATGAGTTTGGACAAACCACAACTAGAATGC |
| polyadenylation | | | | AGTGAAAAAAATGCTTTATTTGTGAAATTTGTGATGCTATTGCTTTATTTGTAACC |
| signal | | | | ATTATAAGCTGCAATAAACAAGTT |
| herpesvirus | 49 | | 4 | CGGCAATAAAAAGACAGAATAAAACGCACGGGTGTTGGGTCGTTTGTTC |
| thymidine | | | | |
| kinase | | | | |
| polyadenylation | | | | |
| signal | | | | |
| SV40 late | 226 | | 0 | CCATACCACATTTGTAGAGGTTTTACTTGCTTTAAAAAACCTCCCACACCTCCCCCT |
| polyadenylation | | | | GAACCTGAAACATAAAATGAATGCAATTGTTGTTGTTAACTTGTTTATTGCAGCTT |
| signal | | | | ATAATGGTTACAAATAAAGCAATAGCATCACAAATTTCACAAATAAAGCATTTTTT |
| | | | | TCACTGCATTCTAGTTGTGGTTTGTCCAAACTCATCAATGTATCTTATCATGTCTGG |
| Human | 416 | | 2 | CATCACATTTAAAAGCATCTCAGCCTACCATGAGAATAAGAGAAAGAAAATGAA |
| Albumin 3′ UTR | | | | GATCAAAAGCTTATTCATCTGTTTTTCTTTTTCGTTGGTGTAAAGCCAACACCCTGT |
| and | | | | CTAAAAAACATAAATTTCTTTAATCATTTTGCCTCTTTTCTCTGTGCTTCAATTAATA |
| Terminator/polyA | | | | AAAAATGGAAAGAATCTAATAGAGTGGTACAGCACTGTTATTTTTCAAAGATGTG |
| Sequence | | | | TTGCTATCCTGAAAATTCTGTAGGTTCTGTGGAAGTTCCAGTGTTCTCTCTTATTCC |
| | | | | ACTTCGGTAGAGGATTTCTAGTTTCTTGTGGGCTAATTAAATAAATCATTAATACT |
| | | | | CTTCTAAGTTATGGATTATAAACATTCAAAATAATATTTTGACATTATGATAATTCT |
| | | | | GAATAAAAGAACAAAAACCATG |
| Human | 415 | | 2 | ATCACATTTAAAAGCATCTCAGCCTACCATGAGAATAAGAGAAAGAAAATGAAG |
| Albumin 3′ UTR | | | | ATCAAAAGCTTATTCATCTGTTTTTCTTTTTCGTTGGTGTAAAGCCAACACCCTGTC |
| and | | | | TAAAAAACATAAATTTCTTTAATCATTTTGCCTCTTTTCTCTGTGCTTCAATTAATAA |
| Terminator/polyA | | | | AAAATGGAAAGAATCTAATAGAGTGGTACAGCACTGTTATTTTTCAAAGATGTGT |
| Sequence | | | | TGCTATCCTGAAAATTCTGTAGGTTCTGTGGAAGTTCCAGTGTTCTCTCTTATTCCA |
| | | | | CTTCGGTAGAGGATTTCTAGTTTCTTGTGGGCTAATTAAATAAATCATTAATACTC |
| | | | | TTCTAAGTTATGGATTATAAACATTCAAAATAATATTTTGACATTATGATAATTCTG |
| | | | | AATAAAAGAACAAAAACCATG |
| CpGfree, Short | 122 | | 0 | TAAGATACATTGATGAGTTTGGACAAACCACAACTAGAATGCAGTGAAAAAAAT |
| SV40 polyA | | | | GCTTTATTTGTGAAATTTGTGATGCTATTGCTTTATTTGTAACCATTATAAGCTGCA |
| ATAAACAAGTT | | | | |
| CpGfree, Short | 133 | | 0 | TGCTTTATTTGTGAAATTTGTGATGCTATTGCTTTATTTGTAACCATTATAAGCTGC |
| SV40 polyA | | | | AATAAACAAGTTAACAACAACAATTGCATTCATTTTATGTTTCAGGTTCAGGGGG |
| | | | | AGGTGTGGGAGGTTTTTTAAA |

In one embodiment, the vector polynucleotide (the ceDNA vector) comprises a pair of two different ITRs selected from the group consisting of: SEQ ID NO:1 and SEQ ID NO:52; and SEQ ID NO:2 and SEQ ID NO:51.In one embodiment of each of these aspects, the vector polynucleotide or the non-viral, capsid-free DNA vectors with covalently-closed ends comprises a pair of ITRs selected from the group consisting of: SEQ ID NO:101 and SEQ ID NO:102; SEQ ID NO:103, and SEQ ID NO:104, SEQ ID NO:105, and SEQ ID NO:106; SEQ ID NO:107, and SEQ ID NO:108; SEQ ID NO:109, and SEQ ID NO:110; SEQ ID NO:111, and SEQ ID NO:112; SEQ ID NO:113 and SEQ ID NO:114; and SEQ ID NO:115 and SEQ ID NO:116. In some embodiments, the ceDNA vectors do not have an ITR that comprises any sequence selected from SEQ ID NOs: 500-529.

The time for harvesting and collecting DNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce DNA-vectors (e.g., TTX-vectors) but before a majority of cells start to die because of the viral toxicity. The DNA-vectors can be isolated using plasmid purification kits such as Qiagen Endo-Free Plasmid kits. Other methods developed for plasmid isolation can be also adapted for DNA-vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

In one embodiment, the capsid free non-viral DNA vector comprises or is obtained from a plasmid comprising a polynucleotide template comprising in this order: a first adeno-associated virus (AAV) inverted terminal repeat (ITR), a nucleotide sequence of interest (for example an expression cassette of an exogenous DNA) and a modified AAV ITR, wherein said template nucleic acid molecule is devoid of AAV capsid protein coding. In a further embodiment, the nucleic acid template of the invention is devoid of viral capsid protein coding sequences (i.e. it is devoid of AAV capsid genes but also of capsid genes of other viruses). In addition, in a particular embodiment, the template nucleic acid molecule is also devoid of AAV Rep protein coding sequences. Accordingly, in a preferred embodiment, the nucleic acid molecule of the invention is devoid of both functional AAV cap and AAV rep genes.

In one embodiment, the ceDNA vector can include an ITR structure that is mutated with respect to the wild type AAV2 ITR disclosed herein, but still retains an operable RBE, trs and RBE′ portion. In some embodiments, the ceDNA vectors do not have an ITR that comprises any sequence selected from SEQ ID NOs: 500-529.

In some embodiments, a transgene encoding an inflammasome antagonist (e.g., any one or more of: an inhibitor of the NLRP3 inflammasome pathway, or an inhibitor of the AIM2 inflammasome pathway, or an inhibitor of caspase 1, or any combination thereof) can also encode a secretory sequence so that the inflammasome antagonist is directed to the Golgi Apparatus and Endoplasmic Reticulum whence the inflammasome antagonist (e.g., any one or more of: an inhibitor of the NLRP3 inflammasome pathway, or an inhibitor of the AIM2 inflammasome pathway, or an inhibitor of caspase 1, or any combination thereof) will be folded into the correct conformation by chaperone molecules as it passes through the ER and out of the cell. Exemplary secretory sequences include, but are not limited to VH-02 (SEQ ID NO: 950) and VK-A26 (SEQ ID NO: 951) and Igκ signal sequence, as well as a Gluc secretory signal that allows the tagged protein to be secreted out of the cytosol, TMD-ST secretory sequence, that directs the tagged protein to the golgi.

Nuclear Localization Sequences: In some embodiments, the ceDNA vector for expression of an e.g. inhibitor of the immune response (e.g., the innate immune response) comprises one or more nuclear localization sequences (NLSs), for example, 1, 2, 3, 4, 5, 6, 7, 8, 9, 10, or more NLSs. In some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present in one or more copies. Non-limiting examples of NLSs are shown in Table 4.

TABLE 4
Nuclear Localization Signals

| | | SEQ ID |
|---|---|---|
| SOURCE | SEQUENCE | NO. |
| SV40 virus large T- | PKKKRKV (encoded by CCCAAGAAGAAGAGGAAGGTG) | 315 |
| antigen | | |
| nucleoplasmin | KRPAATKKAGQAKKKK | 316 |
| c-myc | PAAKRVKLD | 317 |
| | RQRRNELKRSP | 318 |
| hRNPA1 M9 | NQSSNFGPMKGGNFGGRSSGPYGGGGQYFAKPRNQGGY | 319 |
| IBB domain from | RMRIZFKNKGKDTAELRRRRVEVSVELRKAKKDEQILKRRNV | 320 |
| importin-alpha | | |
| myoma T protein | VSRKRPRP | 321 |
| | PPKKARED | 322 |
| human p53 | PQPKKKPL | 323 |
| mouse c-abl IV | SALIKKKKKMAP | 324 |
| influenza virus NS1 | DRLRR | 325 |
| | PKQKKRK | 326 |
| Hepatitis virus | RKLKKKIKKL | 327 |
| delta antigen | | |
| mouse Mx1 | REKKKFLKRR | 328 |
| protein | | |
| human poly(ADP- | KRKGDEVDGVDEVAKKKSKK | 329 |
| ribose) polymerase | | |
| steroid hormone | RKCLQAGMNLEARKTKK | 330 |
| receptors (human) | | |
| glucocorticoid | | |

Regulatory Switches: A molecular regulatory switch is one which generates a measurable change in state in response to a signal. Regulatory switches can also be used to fine tune the expression of an inhibitor of the immune response (e.g., the innate immune response), as described herein, such that the inhibitor of the immune response is expressed as desired, including but not limited to expression of inhibitor of the immune response at a desired expression level or amount, or alternatively, when there is the presence or absence of particular signal, including a cellular signaling event. For instance, as described herein, expression of the inhibitor of the immune response from the ceDNA vector can be turned on or turned off when a particular condition occurs. In some embodiments, the switch is an “ON/OFF” switch that is designed to start or stop (i.e., shut down) expression of an inhibitor of the immune response (e.g., the innate immune response) in the ceDNA vector in a controllable and regulatable fashion. In some embodiments, the switch can include a “kill switch” that can instruct the cell comprising the ceDNA vector to undergo cell programmed death once the switch is activated. Exemplary regulatory switches encompassed for use in a ceDNA vector for expression of an inhibitor of the immune response (e.g., the innate immune response) can be used to regulate the expression of a transgene, and are more fully discussed in International application PCT/US18/49996, which is incorporated herein in its entirety by reference
(i) Binary Regulatory Switches

In some embodiments, the ceDNA vector for expression of an inhibitor of the immune response (e.g., the innate immune response) comprises a regulatory switch that can serve to controllably modulate expression of the infammasome antagonist. For example, the expression cassette located between the ITRs of the ceDNA vector may additionally comprise a regulatory region, e.g., a promoter, cis-element, repressor, enhancer etc., that is operatively linked to the nucleic acid sequence encoding an inhibitor of the immune response (e.g., the innate immune response), where the regulatory region is regulated by one or more cofactors or exogenous agents. By way of example only, regulatory regions can be modulated by small molecule switches or inducible or repressible promoters. Non-limiting examples of inducible promoters are hormone-inducible or metal-inducible promoters. Other exemplary inducible promoters/enhancer elements include, but are not limited to, an RU486-inducible promoter, an ecdysone-inducible promoter, a rapamycin-inducible promoter, and a metallothionein promoter.

(ii) Small Molecule Regulatory Switches

A variety of art-known small-molecule based regulatory switches are known in the art and can be combined with the inhibitor of the immune response (e.g., the innate immune response) as disclosed herein to form a regulatory-switch controlled ceDNA vector. In some embodiments, the regulatory switch can be selected from any one or a combination of: an orthogonal ligand/nuclear receptor pair, for example retinoid receptor variant/LG335 and GRQCIMFI, along with an artificial promoter controlling expression of the operatively linked transgene, such as that as disclosed in Taylor, et al. BMC Biotechnology 10 (2010): 15; engineered steroid receptors, e.g., modified progesterone receptor with a C-terminal truncation that cannot bind progesterone but binds RU486 (mifepristone) (U.S. Pat. No. 5,364,791); an ecdysone receptor from Drosophila and their ecdysteroid ligands (Saez, et al., PNAS, 97(26)(2000), 14512-14517; or a switch controlled by the antibiotic trimethoprim (TMP), as disclosed in Sando R 3^{rd}; Nat Methods. 2013, 10(11):1085-8. In some embodiments, the regulatory switch to control the transgene or expressed by the ceDNA vector is a pro-drug activation switch, such as that disclosed in U.S. Pat. Nos. 8,771,679, and 6,339,070.

(iii) “Passcode” Regulatory Switches

In some embodiments the regulatory switch can be a “passcode switch” or “passcode circuit”. Passcode switches allow fine tuning of the control of the expression of the transgene from the ceDNA vector when specific conditions occur—that is, a combination of conditions need to be present for transgene expression and/or repression to occur. For example, for expression of a transgene to occur at least conditions A and B must occur. A passcode regulatory switch can be any number of conditions, e.g., at least 2, or at least 3, or at least 4, or at least 5, or at least 6 or at least 7 or more conditions to be present for transgene expression to occur. In some embodiments, at least 2 conditions (e.g., A, B conditions) need to occur, and in some embodiments, at least 3 conditions need to occur (e.g., A, B and C, or A, B and D). By way of an example only, for gene expression from a ceDNA to occur that has a passcode “ABC” regulatory switch, conditions A, B and C must be present. Conditions A, B and C could be as follows; condition A is the presence of a condition or disease, condition B is a hormonal response, and condition C is a response to the transgene expression. For example, if the transgene edits a defective EPO gene, Condition A is the presence of Chronic Kidney Disease (CKD), Condition B occurs if the subject has hypoxic conditions in the kidney, Condition C is that Erythropoietin-producing cells (EPC) recruitment in the kidney is impaired; or alternatively, HIF-2 activation is impaired. Once the oxygen levels increase or the desired level of EPO is reached, the transgene turns off again until 3 conditions occur, turning it back on.

In some embodiments, a passcode regulatory switch or “Passcode circuit” encompassed for use in the ceDNA vector comprises hybrid transcription factors (TFs) to expand the range and complexity of environmental signals used to define biocontainment conditions. As opposed to a deadman switch which triggers cell death in the presence of a predetermined condition, the “passcode circuit” allows cell survival or transgene expression in the presence of a particular “passcode”, and can be easily reprogrammed to allow transgene expression and/or cell survival only when the predetermined environmental condition or passcode is present.

Any and all combinations of regulatory switches disclosed herein, e g, small molecule switches, nucleic acid-based switches, small molecule-nucleic acid hybrid switches, post-transcriptional transgene regulation switches, post-translational regulation, radiation-controlled switches, hypoxia-mediated switches and other regulatory switches known by persons of ordinary skill in the art as disclosed herein can be used in a passcode regulatory switch as disclosed herein. Regulatory switches encompassed for use are also discussed in the review article Kis et al., J R Soc Interface. 12: 20141000 (2015), and summarized in Table 1 of Kis. In some embodiments, a regulatory switch for use in a passcode system can be selected from any or a combination of the switches disclosed in Table 11 of Internatioanl Patent Application PCT/US18/49996, which is incorporated herein in its entirety by reference.

(iv). Nucleic Acid-Based Regulatory Switches to Control Transgene Expression

In some embodiments, the regulatory switch to control the expression of an inhibitor of the immune response (e.g., the innate immune response) by the ceDNA is based on a nucleic-acid based control mechanism. Exemplary nucleic acid control mechanisms are known in the art and are envisioned for use. For example, such mechanisms include riboswitches, such as those disclosed in, e.g., US2009/0305253, US2008/0269258, US2017/0204477, WO2018026762A1, U.S. Pat. No. 9,222,093 and EP application EP288071, and also disclosed in the review by Villa J K et al., Microbiol Spectr. 2018 May; 6(3). Also included are metabolite-responsive transcription biosensors, such as those disclosed in WO2018/075486 and WO2017/147585. Other art-known mechanisms envisioned for use include silencing of the transgene with an siRNA or RNAi molecule (e.g., miR, shRNA). For example, the ceDNA vector can comprise a regulatory switch that encodes a RNAi molecule that is complementary to the part of the transgene expressed by the ceDNA vector. When such RNAi is expressed even if the transgene (e.g., an inflammasome antagonist (e.g., inhibitor of one or more of NLRP3 and/or AIM2 inflammasome pathway, or a caspase 1 inhibitor)) is expressed by the ceDNA vector, it will be silenced by the complementary RNAi molecule, and when the RNAi is not expressed when the transgene is expressed by the ceDNA vector the transgene (e.g., an inflammasome antagonist) is not silenced by the RNAi.

In some embodiments, the regulatory switch is a tissue-specific self-inactivating regulatory switch, for example as disclosed in US2002/0022018, whereby the regulatory switch deliberately switches transgene (e.g., an inflammasome antagonist) off at a site where transgene expression might otherwise be disadvantageous. In some embodiments, the regulatory switch is a recombinase reversible gene expression system, for example as disclosed in US2014/0127162 and U.S. Pat. No. 8,324,436.

(v). Post-Transcriptional and Post-Translational Regulatory Switches.

In some embodiments, the regulatory switch to control the expression of inhibitor of the immune response (e.g., the innate immune response) by the ceDNA vector is a post-transcriptional modification system. For example, such a regulatory switch can be an aptazyme riboswitch that is sensitive to tetracycline or theophylline, as disclosed in US2018/0119156, GB201107768, WO2001/064956A3, EP Patent 2707487 and Beilstein et al., ACS Synth. Biol., 2015, 4 (5), pp 526-534; Zhong et al., Elife. 2016 Nov. 2; 5. pii: e18858. In some embodiments, it is envisioned that a person of ordinary skill in the art could encode both the transgene and an inhibitory siRNA which contains a ligand sensitive (OFF-switch) aptamer, the net result being a ligand sensitive ON-switch.

(vi). Other Exemplary Regulatory Switches

Any known regulatory switch can be used in the ceDNA vector to control the expression of an inhibitor of the immune response (e.g., the innate immune response) by the ceDNA vector, including those triggered by environmental changes. Additional examples include, but are not limited to; the BOC method of Suzuki et al., Scientific Reports 8; 10051 (2018); genetic code expansion and a non-physiologic amino acid; radiation-controlled or ultra-sound controlled on/off switches (see, e.g., Scott S et al., Gene Ther. 2000 July; 7(13):1121-5; U.S. Pat. Nos. 5,612,318; 5,571,797; 5,770,581; 5,817,636; and WO1999/025385A1. In some embodiments, the regulatory switch is controlled by an implantable system, e.g., as disclosed in U.S. Pat. No. 7,840,263; US2007/0190028A1 where gene expression is controlled by one or more forms of energy, including electromagnetic energy, that activates promoters operatively linked to the transgene in the ceDNA vector.

In some embodiments, a regulatory switch envisioned for use in the ceDNA vector is a hypoxia-mediated or stress-activated switch, e.g., such as those disclosed in WO1999060142A2, U.S. Pat. Nos. 5,834,306; 6,218,179; 6,709,858; US2015/0322410; Greco et al., (2004) Targeted Cancer Therapies 9, 5368, as well as FROG, TOAD and NRSE elements and conditionally inducible silence elements, including hypoxia response elements (HREs), inflammatory response elements (IREs) and shear-stress activated elements (SSAEs), e.g., as disclosed in U.S. Pat. No. 9,394,526. Such an embodiment is useful for turning on expression of the transgene from the ceDNA vector after ischemia or in ischemic tissues, and/or tumors.

(vii). Kill Switches

Other embodiments described herein relate to a ceDNA vector for expression of an inhibitor of the immune response (e.g., the innate immune response) as described herein comprising a kill switch. A kill switch as disclosed herein enables a cell comprising the ceDNA vector to be killed or undergo programmed cell death as a means to permanently remove an introduced ceDNA vector from the subject's system. It will be appreciated by one of ordinary skill in the art that use of kill switches in the ceDNA vectors for expression of an inhibitor of the immune response (e.g., the innate immune response) would be typically coupled with targeting of the ceDNA vector to a limited number of cells that the subject can acceptably lose or to a cell type where apoptosis is desirable (e.g., cancer cells). In all aspects, a “kill switch” as disclosed herein is designed to provide rapid and robust cell killing of the cell comprising the ceDNA vector in the absence of an input survival signal or other specified condition. Stated another way, a kill switch encoded by a ceDNA vector for expression of an inflammasome antagonist as described herein can restrict cell survival of a cell comprising a ceDNA vector to an environment defined by specific input signals. Such kill switches serve as a biological biocontainment function should it be desirable to remove the ceDNA vector expression of an inflammasome antagonist in a subject or to ensure that it will not express the encoded inflammasome antagonist.

Other kill switches known to a person of ordinary skill in the art are encompassed for use in the ceDNA vector for expression of an inhibitor of the immune response (e.g., the innate immune response) as disclosed herein, e.g., as disclosed in U52010/0175141; U52013/0009799; U52011/0172826; U52013/0109568, as well as kill switches disclosed in Jusiak et al., Reviews in Cell Biology and molecular Medicine; 2014; 1-56; Kobayashi et al., PNAS, 2004; 101; 8419-9; Marchisio et al., Int. Journal of Biochem and Cell Biol., 2011; 43; 310-319; and in Reinshagen et al., Science Translational Medicine, 2018, 11.

Accordingly, in some embodiments, the ceDNA vector for expression of inhibitor of the immune response (e.g., the innate immune response) can comprise a kill switch nucleic acid construct, which comprises the nucleic acid encoding an effector toxin or reporter protein, where the expression of the effector toxin (e.g., a death protein) or reporter protein is controlled by a predetermined condition. For example, a predetermined condition can be the presence of an environmental agent, such as, e.g., an exogenous agent, without which the cell will default to expression of the effector toxin (e.g., a death protein) and be killed. In alternative embodiments, a predetermined condition is the presence of two or more environmental agents, e.g., the cell will only survive when two or more necessary exogenous agents are supplied, and without either of which, the cell comprising the ceDNA vector is killed.

In some embodiments, the ceDNA vector for expression of an inhibitor of the immune response (e.g., the innate immune response) is modified to incorporate a kill-switch to destroy the cells comprising the ceDNA vector to effectively terminate the in vivo expression of the transgene being expressed by the ceDNA vector (e.g., expression of an inflammasome antagonist). Specifically, the ceDNA vector is further genetically engineered to express a switch-protein that is not functional in mammalian cells under normal physiological conditions. Only upon administration of a drug or environmental condition that specifically targets this switch-protein, the cells expressing the switch-protein will be destroyed thereby terminating the expression of the therapeutic protein or peptide. For instance, it was reported that cells expressing HSV-thymidine kinase can be killed upon administration of drugs, such as ganciclovir and cytosine deaminase. See, for example, Dey and Evans, Suicide Gene Therapy by Herpes Simplex Virus-1 Thymidine Kinase (HSV-TK), in Targets in Gene Therapy, edited by You (2011); and Beltinger et al., Proc. Natl. Acad. Sci. USA 96(15):8699-8704 (1999). In some embodiments the ceDNA vector can comprise a siRNA kill switch referred to as DISE (Death Induced by Survival gene Elimination) (Murmann et al., Oncotarget. 2017; 8:84643-84658. Induction of DISE in ovarian cancer cells in vivo).

In another embodiment, the inhibitor of the immune response (e.g., the innate immune response) expressed from the ceDNA vectors further comprises an additional functionality, such as fluorescence, enzyme activity, secretion signal or immune cell activator.

In some embodiments, the ceDNA encoding the inhibitor of the immune response (e.g., the innate immune response) can further comprise a linker domain, for example. As used herein “linker domain” refers to an oligo- or polypeptide region from about 2 to 100 amino acids in length, which links together any of the domains/regions of the inflammasome antagonist as described herein. In some embodiment, linkers can include or be composed of flexible residues such as glycine and serine so that the adjacent protein domains are free to move relative to one another. Longer linkers may be used when it is desirable to ensure that two adjacent domains do not sterically interfere with one another. Linkers may be cleavable or non-cleavable. Examples of cleavable linkers include 2A linkers (for example T2A), 2A-like linkers or functional equivalents thereof and combinations thereof. The linker can be a linker region is T2A derived from Thosea asigna virus.

### IV. Method of Production of a ceDNA Vector

### A. Production in General

Certain methods for the production of a ceDNA vector for expression of e.g. an inhibitor of the immune response (e.g., the innate immune response) comprising an asymmetrical ITR pair or symmetrical ITR pair as defined herein is described in section IV of International application PCT/US18/49996 filed Sep. 7, 2018, which is incorporated herein in its entirety by reference. In some embodiments, a ceDNA vector for expression of an inflammasome antagonist as disclosed herein can be produced using insect cells, as described herein. In alternative embodiments, a ceDNA vector for expression of an inflammasome antagonist as disclosed herein can be produced synthetically and in some embodiments, in a cell-free method, as disclosed on International Application PCT/US19/14122, filed Jan. 18, 2019, which is incorporated herein in its entirety by reference.

As described herein, in one embodiment, a ceDNA vector for expression of an inhibitor of the immune response (e.g., the innate immune response) e.g. can be obtained, for example, by the process comprising the steps of: a) incubating a population of host cells (e.g. insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g. AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the invention provides for use of host cell lines that have stably integrated the DNA vector polynucleotide expression template (ceDNA template) into their own genome in production of the non-viral DNA vector, e.g. as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

In one embodiment, the host cells used to make the ceDNA vectors for expression of an inhibitor of the immune response (e.g., the innate immune response) e.g. as described herein are insect cells, and baculovirus is used to deliver both the polynucleotide that encodes Rep protein and the non-viral DNA vector polynucleotide expression construct template for ceDNA, e.g., as described in FIGS. 4A-4D and Example 1. In some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. In one embodiment, cells are grown under sufficient conditions and harvested a sufficient time after baculoviral infection to produce ceDNA vectors but before a majority of cells start to die because of the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. In one embodiment, ceDNA vectors are purified as DNA molecules. In another embodiment, the ceDNA vectors are purified as exosomes or microparticles.

The presence of the ceDNA vector for expression of an inhibitor of the immune response (e.g., the innate immune response) can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

B. ceDNA Plasmid

A ceDNA-plasmid is a plasmid used for later production of a ceDNA vector for expression of an inhibitor of the immune response (e.g., the innate immune response). e.g. In some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. In some embodiments, the expression cassette flanked by the ITRs comprises a cloning site for introducing an exogenous sequence. The expression cassette replaces the rep and cap coding regions of the AAV genomes.

In one aspect, a ceDNA vector for expression of an inhibitor of the immune response (e.g., the innate immune response) e.g. is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs are have the same modifications (i.e., they are inverse complement or symmetric relative to each other).

In a further embodiment, the ceDNA-plasmid system is devoid of viral capsid protein coding sequences (i.e. it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present invention can be generated using natural nucleotide sequences of the genomes of any AAV serotypes well known in the art. In one embodiment, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at www web address: oesys.springer.de/viruses/database/mkchapter.asp?virID=42.04.)(note—references to a URL or database refer to the contents of the URL or database as of the effective filing date of this application) In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. In one embodiment, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAV0) vector for expression of an inflammasome antagonist (e.g., inhibitor of one or more of NLRP3 and/or AIM2 inflammasome pathway, or a caspase 1 inhibitor) is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

C. Exemplary Method of Making the ceDNA Vectors from ceDNA Plasmids

Methods for making capsid-less ceDNA vectors for expression of an inhibitor of the immune response (e.g., the innate immune response) are also provided herein, notably a method with a sufficiently high yield to provide sufficient vector for in vivo experiments.

In some embodiments, a method for the production of a ceDNA vector for expression of an inhibitor of the immune response (e.g., the innate immune response) e.g. comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

D. Cell lines:

Host cell lines used in the production of a ceDNA vector for expression of an inhibitor of the immune response (e.g., the innate immune response) e.g. can include insect cell lines derived from Spodoptera frugiperda, such as Sf9, Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

E. Isolating and Purifying ceDNA Vectors:

Examples of the process for obtaining and isolating ceDNA vectors are described in FIGS. 1-7 and the specific examples below. ceDNA-vectors for expression of an inhibitor of the immune response (e.g., the innate immune response) e.g. disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode an inflammasome antagonist, or plasmids encoding one or more REP proteins.

In one aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for expression of an inhibitor of the immune response (e.g., the innate immune response) e.g. are described herein. Expression constructs used for generating a ceDNA vector for expression of an inhibitor of the immune response (e.g., the innate immune response) as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for expression of an inflammasome antagonist can be generated from the cells stably transfected with a construct comprising a sequence encoding the AAV Rep protein (Rep78/52) delivered in Rep-plasmids, Rep-bacmids, or Rep-baculovirus. ceDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for expression of an inhibitor of the immune response (e.g., the innate immune response) as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g. SARTOBIND Q®) which retains nucleic acids, and then eluting (e.g. with a 1.2 M NaCl solution) and performing a further chromatographic purification on a gel filtration column (e.g. 6 fast flow GE). The capsid-free AAV vector is then recovered by, e.g., precipitation.

In some embodiments, ceDNA vectors for expression of an inhibitor of the immune response (e.g., the innate immune response) can also be purified in the form of exosomes, or microparticles. It is known in the art that many cell types release not only soluble proteins, but also complex protein/nucleic acid cargoes via membrane microvesicle shedding (Cocucci et al., 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore®, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the invention herein relates to methods of purifying ceDNA vectors from host cell lines that have stably integrated a ceDNA construct into their own genome. In one embodiment, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs using the method described in the Examples. The ceDNA is confirmed by a characteristic band pattern in the gel (see, FIG. 5A).

### V. Pharmaceutical Compositions and Formulations

The present invention contemplates pharmaceutical compositions and formulations comprising a therapeutic nucleic acid and one or more inhibitors of the immune response (e.g., the innate immune response) as described herein. In some embodiments, the pharmaceutical composition comprising a therapeutic nucleic acid and one or more inhibitors of the immune response (e.g., the innate immune response) may include a pharmaceutically acceptable excipient or carrier. According to some embodiments, the pharmaceutical composition comprises a closed-ended DNA vector, e.g., ceDNA vector as described herein and a rapamycin or rapamycin analogue, and a pharmaceutically acceptable carrier or diluent. According to some embodiments, the pharmaceutical composition comprises a closed-ended DNA vector, e.g., ceDNA vector as described herein and a TLR inhibitor (e.g., a TLR9 inhibitor), and a pharmaceutically acceptable carrier or diluent. According to some embodiments, the pharmaceutical composition comprises a closed-ended DNA vector, e.g., ceDNA vector as described herein and a cGAS inhibitor, and a pharmaceutically acceptable carrier or diluent. According to some embodiments, the pharmaceutical composition comprises a closed-ended DNA vector, e.g., ceDNA vector as described herein and an inflammasome antagonist (e.g., any one or more of: an inhibitor of the NLRP3 inflammasome pathway, or an inhibitor of the AIM2 inflammasome pathway, or an inhibitor of caspase 1, or any combination thereof), and a pharmaceutically acceptable carrier or diluent.

The DNA-vectors disclosed herein can be incorporated into pharmaceutical compositions suitable for administration to a subject for in vivo delivery to cells, tissues, or organs of the subject, including, in some embodiments, the pharmaceutical compositions comprising the inhibitors of the immune response (e.g., innate immune response) as described herein. Typically, the pharmaceutical composition comprises the DNA-vectors disclosed herein and a pharmaceutically acceptable carrier. For example, the TTX-vectors of the invention can be incorporated into a pharmaceutical composition suitable for a desired route of therapeutic administration (e.g., parenteral administration). Passive tissue transduction via high pressure intravenous or intraarterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high TTX-vector concentration. Sterile injectable solutions can be prepared by incorporating the TTX-vector compound in the required amount in an appropriate buffer with one or a combination of ingredients enumerated above, as required, followed by filtered sterilization.

Pharmaceutically active compositions comprising a TTX-vector can be formulated to deliver a transgene in the nucleic acid to the cells of a recipient, resulting in the therapeutic expression of the transgene therein. The composition can also include a pharmaceutically acceptable carrier.

The compositions and vectors provided herein can be used to deliver a transgene for various purposes. In some embodiments, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. In some embodiments, the transgene encodes one or more peptides, polypeptides, or proteins, which are useful for the treatment or prevention of disease states in a mammalian subject. The transgene can be transferred (e.g., expressed in) to a patient in a sufficient amount to treat a disease associated with reduced expression, lack of expression or dysfunction of the gene. In some embodiments, the transgene is a gene editing molecule (e.g., nuclease). In certain embodiments, the nuclease is a CRISPR-associated nuclease (Cas nuclease).

According to some embodiments, the pharmaceutically active compositions described herein can be administered in combination with an antihistamine or a a steroid. According to some embodiments, the antihistamine or steroid are administered in the same composition as the pharmaceutically active compositions described herein. According to some embodiments, the antihistamine or steroid are administered in a separate composition as the pharmaceutically active compositions described herein. According to some embodiments, the antihistamine or steroid are administered simultaneously with the pharmaceutically active composition. According to some embodiments, the antihistamine or steroid are administered sequentially with the pharmaceutically active composition. Any antihistamine known in the art can be employed in the embodiments herein. According to some embodiments, the antihistamine is one or more of ompheniramine, buclizine, chlorpheniramine, cinnarizine, clemastine, cyclizine, cyproheptadine, diphenhydramine, diphenylpyraline, doxylamine, meclozine, pheniramine, promethazine, triprolidine, acrivastine, astemizole, cetirizine, desloratadine, fexofenadine, levocetirizine, loratadine, mizolastine, terfenadine, a pharmaceutically acceptable salt thereof, or a combination thereof. Any steroid known in the art can be employed in the embodiments herein. According to some embodiments, the steroid is one or more of t least one of fluoxymesteron, mesterolone, methandrostenolone, nandrolone-undecanoate, nandrolone-cyplonate, oxandrolone, oxymetholone, nandrolone-hexyloxy phenylpropionate, testosterone, prednisone, cortisol, cortisone, prednisolone, dexamethasone, betamethasone, triamcinolone, beclomethasone, fludrocortisone, deoxy corticosterone, aldosterone and stanozolol.

Pharmaceutical compositions for therapeutic purposes typically must be sterile and stable under the conditions of manufacture and storage. Sterile injectable solutions can be prepared by incorporating the ceDNA vector compound in the required amount in an appropriate buffer with one or a combination of ingredients enumerated above, as required, followed by filtered sterilization.

### Unit Dosage

According to some embodiments, the pharmaceutical compositions can be presented in unit dosage form. A unit dosage form will typically be adapted to one or more specific routes of administration of the pharmaceutical composition. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### IV. Admiration and Dosing

The disclosure provided herein describes methods to prevent, reduce or eliminate unwanted immune response (e.g., innate immune response) in a subject (e.g., a human subject) by administering to the subject at least one inhibitor of the immune response (e.g., innate immune response) as described herein and a nucleic acid (e.g. a therapeutic nuclide acid, a nucleic acid used for research purposes), wherein the administrations of the inhibitor of the immune response (e.g., innate immune response) and the administration of the nucleic acid are correlated in time so as to provide a modulation in an immune response (e.g., innate immune response) when the administration of the two agents are provided in combination. These two agents can be administered at the same time in a co-formulation, at the same time in different formulations, or they can be administered separately at different times.

In one embodiment, the expressed inhibitor of the immune response (e.g., the innate immune response)r, as disclosed herein, does not cause an immune system reaction, rather it suppresses the innate immune system in the subject by at least 10%, or 20%, or 30%, or 40%, or 50%, or 60% or 70% or 80% or 90% or 95%, or 98%, or 99% or 100%, as compared to the absence of administration of a ceDNA vector expressing the inhibitor.

The technology described herein is directed in general to methods for co-administering a closed-ended DNA vectors to a subject with one or more inhibitors of the immune response, e.g., the innate immune response), selected from one or more, or a combination of, rapamycin or a rapamycin analogues, inhibitors of TLR (e.g., TLR9), inhibitors of cGAS, and one or more inflammasome antagonists (e.g., any one or more of: an inhibitor of the NLRP3 inflammasome pathway, or an inhibitor of the AIM2 inflammasome pathway, or an inhibitor of caspase 1, or any combination thereof), as described herein. In some embodiments, a close-ended DNA vector includes, but is not limited to, ceDNA vectors as disclosed herein, and mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). (see e.g., WO2010/0086626, the contents of which is incorporated by reference herein in its entirety). According to some embodiments, the inhibitors of the innate immune response and the nucleic acids can be administered to the subject or patient in any combination. For example, one or more inhibitors of the immune response (e.g., innate immune response) may be administered. According to some embodiments, the subject or patient is administered an inhibitor of the immune response (e.g., the innate immune response) as described herein, and the nucleic acids (e.g., minicircle, minigene, ministring covalently closed DNA, doggybone (dbDNA™) DNA, dumbbell shaped DNA, linear closed-ended duplex DNA (ceDNA and CELiD), plasmid based circular vector, antisense oligonucleotide (ASO), RNAi, siRNA, mRNA, etc.). According to some embodiments, the subject or patient is administered rapamycin or rapamycin analogues, one or more TLR9 inhibitors and the nucleic acids. According to some embodiments, the subject or patient is administered rapamycin or rapamycin analogues, one of more cGAS inhibitors and the nucleic acids. According to some embodiments, the subject or patient is administered rapamycin or rapamycin analogues, one or more inflammasome antagonists, and the nucleic acids. According to some embodiments, the subject or patient is administered rapamycin or rapamycin analogues, one or more TLR9 inhibitors, one or more cGAS inhibitors and the nucleic acids. According to some embodiments, the subject or patient is administered rapamycin or rapamycin analogues, one or more TLR9 inhibitors, one or more inflammasome antagonists and the nucleic acids. According to some embodiments, the subject or patient is administered one or more TLR9 inhibitors, one or more cGAS inhibitors and a ceDNA vector comprising the nucleic acids. According to some embodiments, the subject or patient is administered one or more TLR9 inhibitors, one or more cGAS inhibitors, one or more inflammasome antagonists and the nucleic acids. According to some embodiments, the subject or patient is administered rapamycin or rapamycin analogues, one or more TLR9 inhibitors, one or more cGAS inhibitors, one or more inflammasome antagonists and the nucleic acids.

In some embodiments, a subject may be administered one or more inhibitors of the immune response (e.g., innate immune response) and one or more nucleic acids (e g, minicircle, minigene, ministring covalently closed DNA, doggybone (dbDNA™) DNA, dumbbell shaped DNA, linear closed-ended duplex DNA (ceDNA and CELiD), plasmid based circular vector, antisense oligonucleotide (ASO), RNAi, siRNA, mRNA, etc.) concomitantly. For example, the method may comprise administering to a subject an inhibitor of the immune response (e.g., innate immune response) and a nucleic acid therapeutic as two separate formulations but concomitantly. In another example, the method may comprise simultaneously administering to a subject an inhibitor of the immune response (e.g., innate immune response) and a therapeutic nucleic acid in one formulation at the same time.

In some embodiment, a subject may be administered one or more inhibitors of the immune response (e.g., innate immune response) and one or more nucleic acids (e.g., minicircle, minigene, ministring covalently closed DNA, doggybone (dbDNA™) DNA, dumbbell shaped DNA, linear closed-ended duplex DNA (ceDNA and CELiD), plasmid based circular vector, antisense oligonucleotide (ASO), RNAi, siRNA, mRNA, etc.) sequentially. For example, the inhibitor of the immune response (e.g., innate immune response) may be administered prior to administration of a therapeutic nucleic acid.

In cases of sequential administration, there may be a delay period between administration of the one or more inhibitor of the immune response (e.g., innate immune response) and TNAs. For example, the inhibitor of the immune response (e.g., innate immune response) may be administered hours, days, or weeks prior to administration of the TNA (e.g., at least 30 minutes, at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least about 2 days, at least about 3 days, at least about 4 days, at least about 5 days, at least about 6 days, at least about 1 week, at least about 2 weeks, at least about 3 weeks, and at least about 4 weeks prior to the administration of a nucleic acid). In some embodiments, an inhibitor of the immune response (e.g., innate immune response) may be administered about thirty (30) minutes prior to the administration of a TNA. In some embodiments, an inhibitor of the immune response (e.g., innate immune response) may be administered about one (1) hour prior to the administration of a nucleic acid. In some embodiments, an inhibitor of the immune response (e.g., innate immune response) can be administered about two (2) hours prior to the administration of a nucleic acid. In some embodiments, an inhibitor of the immune response (e.g., innate immune response) can be administered about three (3) hours prior to the administration of a nucleic acid. In some embodiments, an inhibitor of the immune response (e.g., innate immune response) can be administered about four (4) hours prior to the administration of a nucleic acid. In some embodiments, an inhibitor of the immune response (e.g., innate immune response) can be administered about five (5) hours prior to the administration of a nucleic acid. In some embodiments, an inhibitor of the immune response (e.g., innate immune response) can be administered about six (6) hours prior to the administration of a nucleic acid. In some embodiments, an inhibitor of the immune response (e.g., innate immune response) can be administered about seven (7) hours prior to the administration of a nucleic acid. In some embodiments, an inhibitor of the immune response (e.g., innate immune response) can be administered about eight (8) hours prior to the administration of a nucleic acid. In some embodiments, an inhibitor of the immune response (e.g., innate immune response) can be administered about nine (9) hours prior to the administration of a nucleic acid. In some embodiments, an inhibitor of the immune response (e.g., innate immune response) can be administered about ten (10) hours prior to the administration of a nucleic acid.

In one embodiment, an inhibitor of the immune response (e.g., innate immune response) is administered no more than about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours before the administration of a nucleic acid. In some embodiments, an inhibitor of the immune response (e.g., innate immune response) can be administered no more than about 1 day, about 2 days, about 3 days, about 4 days, about 6 days, or about 7 days before the administration of a nucleic acid.

In some embodiments, an inhibitor of the immune response (e.g., innate immune response) can be administered about 30 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of a nucleic acid. In some embodiments, an inhibitor of the immune response (e.g., innate immune response) can be administered about 1 day, about 2 days, about 3 days, about 4 days, about 6 days, or about 7 days after the administration of a nucleic acid.

In one embodiment, an inhibitor of the immune response (e.g., innate immune response) is administered no more than about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or 24 hours after the administration of a nucleic acid. In some embodiments, an inhibitor of the immune response (e.g., innate immune response) can be administered no more than about 1 day, about 2 days, about 3 days, about 4 days, about 6 days, or about 7 days after the administration of a nucleic acid.

In some embodiments, one or more inhibitor of the immune response (e.g., innate immune response) can be administered multiple times before, concurrently with, and/or after the administration of a nucleic acid.

In some embodiments, a nucleic acid (e.g., a ceDNA vector) can be administered as a single dose or as multiple doses. According to some embodiments, more than one dose can be administered to a subject. Multiple doses can be administered as needed, because the ceDNA vector does not elicit an anti-capsid host immune response due to the absence of a viral capsid. According to some embodiments the number of doses administered can, for example, be between 2-10 or more doses, for example 2, 3, 4, 5, 6, 7, 8, 9, 10 or more.

In some embodiments, a nucleic acid can be administered and re-dosed multiple times in conjunction with one or more inhibitors of the immune response (e.g., innate immune response) disclosed herein. For example, the therapeutic nucleic acid can be administered on day 0 with one or more inhibitors of the immune response that is administered before, after or at the same time with the administration the nucleic acid in a first dosing regimen. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the nucleic acid, preferably with one or more inhibitors of the immune response (e.g., innate immune response) disclosed herein.

According to some embodiments, re-dosing of the nucleic acid results in an increase in expression of the nucleic acid. According to some embodiments, the increase of expression of the nucleic acid after re-dosing, compared to the expression of the nucleic acid after the first dose is about 0.5-fold to about 10-fold, about 1-fold to about 5-fold, about 1-fold to about 2-fold, or about 0.5-fold, about 1-fold, about 2-fold, about 3-fold, about 4-fold, about 5-fold, about 6-fold, about 7-fold, about 8-fold, about 9-fold or about 10-fold higher after re-dosing of the nucleic acid.

Generally, the dosage will vary with the particular characteristics of the ceDNA vector, expression efficiency and with the age, condition, and sex of the patient. The dosage can be determined by one of skill in the art and, unlike traditional AAV vectors, can also be adjusted by the individual physician in the event of any complication because ceDNA vectors do not comprise immune activating capsid proteins that prevent repeat dosing.

According to some embodiments, more than one administration (e.g., two, three, four or more administrations) of a nucleic acid (e.g., a ceDNA vector) for expression of a protein as disclosed herein may be employed to achieve a desired level of gene expression over a period of various intervals, e.g., daily, weekly, monthly, yearly, etc.

According to any of the embodiments disclosed herein, the nucleic acid may be a therapeutic nucleic acid.

### Therapeutic Effect

The efficacy of a ceDNA vector expressing an inhibitor of the immune response (e.g., the innate immune response), as disclosed herein, for suppressing or reducing the innate immune system, can be determined by the skilled clinician. However, a treatment is considered “effective treatment,” as the term is used herein, if any one or all of the signs or symptoms of the innate immune system are reduced and/or are altered in a beneficial manner, or other clinically accepted symptoms or markers of disease are improved, or ameliorated, e.g., by at least 10% after treatment with a ceDNA vector encoding an inhibitor of the immune response (e.g., the innate immune response), as disclosed herein. Exemplary markers and symptoms are discussed in the Examples herein. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the disease or disorder; or (2) relieving the disease, e.g., causing regression of symptoms; and (3) preventing or reducing the likelihood of the development of the disease, or preventing secondary diseases/disorders associated with the disease, such as liver or kidney failure. An effective amount for the treatment of a disease means that amount which, when administered to a mammal in need thereof, is sufficient to result in effective treatment as that term is defined herein, for that disease.

Efficacy of an agent can be determined by assessing physical indicators that are particular to a given disease. Standard methods of analysis of disease indicators are known in the art. For example, physical indicators for the innate immune system include for example, without limitation, soluble CD14 (sCD14) and IL-18, IL-22, in the plasma or blood, inflammasome proteins, such as AIM2, NLRP3, NLRP1, ASC, and caspase-1 in the CSF or blood, activation of cytokine pathways can be used as functional readout of activation of the NLRP3 and/or AIM2 inflammasome pathway, or a caspase 1 activation, and include biomarkers such as, but not limited to: interleukin (IL)-1β, IL-6, IL-8, IL-18, interferon (IFN)-γ, interferon (IFN)-α, monocyte chemoattractant protein (MCP)-1, and/or tumor necrosis factor (TNF)-α.

In one embodiment, the ceDNA vector comprises a nucleic acid sequence to express an inhibitor of the immune response (e.g., the innate immune response), as disclosed herein, e.g., that is functional for the suppression of the innate immune system. In a preferred embodiment, an inhibitor of the immune response (e.g., the innate immune response), as disclosed herein, e.g., as disclosed herein, does not cause an immune system reaction, rather, it suppresses or reduces the immune system in the subject.

Pharmaceutical compositions for therapeutic purposes typically must be sterile and stable under the conditions of manufacture and storage. The composition can be formulated as a solution, microemulsion, dispersion, liposomes, or other ordered structure suitable to high closed-ended DNA vector, e.g. ceDNA vector concentration. Sterile injectable solutions can be prepared by incorporating the ceDNA vector in the required amount in an appropriate buffer with one or a combination of ingredients enumerated above, as required, followed by filtered sterilization.

A closed-ended DNA vector, including a ceDNA vector, and an inhibitor of the immune response (e.g., the innate immune response) as disclosed herein, can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

In some aspects, the methods provided herein comprise delivering one or more closed-ended DNA vector, including a ceDNA vector, and an inhibitor of the immune response (e.g., the innate immune response) as described herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, a closed-ended DNA vector, including a ceDNA vector, and rapamycin or a rapamycin analogue as described herein can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering a closed-ended DNA vector, including a ceDNA vector, and an inhibitor of the immune response (e.g., innate immune response) as described herein, to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids and closed-ended DNA vector, including a ceDNA vector as described herein can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific®), Pro-Ject Reagent (Thermo Fisher Scientific®), TRANSPASS™ P Protein Transfection Reagent (New England Biolabs®), CHARIOT™ Protein Delivery Reagent (Active Motif), PROTEOJUICE™ Protein Transfection Reagent (EMD Millipore®), 293fectin, LIPOFECTAMINE™ 2000, LIPOFECTAMINE™ 3000 (Thermo Fisher Scientific®), LIPOFECTAMINE™ (Thermo Fisher Scientific®), LIPOFECTIN™ (Thermo Fisher Scientific®), DMRIE-C, CELLFECTIN™ (Thermo Fisher Scientific®), OLIGOFECTAMINE™ (Thermo Fisher Scientific®), LIPOFECTACE™, FUGENE™ (Roche®, Basel, Switzerland), FUGENE™ HD (Roche®), TRANSFECTAM™ (Transfectam, Promega®, Madison, Wis.), TFX-10™ (Promega®), TFX-20™ (Promega®), TFX-50™ (Promega®), TRANSFECTIN™ (BioRad®, Hercules, Calif.), SILENTFECT™ (Bio-Rad®), Effectene™ (Qiagen®, Valencia, Calif.), DC-chol (Avanti Polar Lipids), GENEPORTER™ (Gene Therapy Systems®, San Diego, Calif.), DHARMAFECT 1™ (Dharmacon®, Lafayette, Colo.), DHARMAFECT 2™ (Dharmacon®), DHARMAFECT 3™ (Dharmacon®), DHARMAFECT 4™ (Dharmacon®), ESCORT™ III (Sigma®, St. Louis, Mo.), and ESCORT™ IV (Sigma Chemical Co.). Nucleic acids, such as ceDNA, can also be delivered to a cell via microfluidics methods known to those of skill in the art.

A closed-ended DNA vector, including a ceDNA vector, and an inhibitor of the immune response (e.g. The innate immune response) as described herein, can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of a closed-ended DNA vector, including a ceDNA vector, and an inhibitor of the innate immune response as described herein, can be delivered into hematopoietic stem cells, for example, by the methods as described, for example, in U.S. Pat. No. 5,928,638.

A closed-ended DNA vector, including a ceDNA vector and an inhibitor of the immune response (e.g., innate immune response) as described herein, can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations are disclosed in International Application PCT/US2018/050042, filed on Sep. 7, 2018 and in International application PCT/US2018/064242, filed on Dec. 6, 2018, e.g., see the section entitled “Pharmaceutical Formulations”.

Various delivery methods known in the art or modifications thereof can be used to deliver a closed-ended DNA vector, including a ceDNA vector, and an inhibitor of the immune response (e.g., the innate immune response) as described herein, in vitro or in vivo. For example, in some embodiments, ceDNA vectors are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. In some cases, a ceDNA vector alone is directly injected as naked DNA into skin, thymus, cardiac muscle, skeletal muscle, or liver cells. In some cases, a ceDNA vector is delivered by gene gun. Gold or tungsten spherical particles (1-3 μm diameter) coated with capsid-free AAV vectors can be accelerated to high speed by pressurized gas to penetrate into target tissue cells.

Compositions comprising a closed-ended DNA vector, including a ceDNA vector, and rapamycin or a rapamycin analogue as described herein, and a pharmaceutically acceptable carrier are specifically contemplated herein. In some embodiments, the ceDNA vector is formulated with a lipid delivery system, for example, liposomes as described herein. In some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods or ultrasound.

In some cases, a closed-ended DNA vector, including a ceDNA vector, and one or more inhibitors of the immune response (e.g., the innate immune response) as described herein, is delivered by hydrodynamic injection, which is a simple and highly efficient method for direct intracellular delivery of any water-soluble compounds and particles into internal organs and skeletal muscle in an entire limb.

In some cases, a closed-ended DNA vector, including a ceDNA vector, and one or more inhibitors of the immune response (e.g., the innate immune response) as described herein, is delivered by ultrasound by making nanoscopic pores in membrane to facilitate intracellular delivery of DNA particles into cells of internal organs or tumors, so the size and concentration of the closed-ended DNA vector have a great role in efficiency of the system. In some cases, closed-ended DNA vectors, including a ceDNA vector, and one or more inhibitors of the immune response (e.g., the innate immune response) as described herein, are delivered by magnetofection by using magnetic fields to concentrate particles containing nucleic acid into the target cells.

In some cases, chemical delivery systems can be used, for example, by using nanomeric complexes, which include compaction of negatively charged nucleic acid by polycationic nanomeric particles, belonging to cationic liposome/micelle or cationic polymers. Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

In some embodiments, a closed-ended DNA vector, including a ceDNA vector, and one or more inhibitors of the immune response (e.g., the innate immune response) as described herein, is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). Some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present invention.

### B. Microparticle/Nanoparticles:

In some embodiments, a closed-ended DNA vector, including a ceDNA vector, and rapamycin or a rapamycin analogue as described herein, is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507. In some embodiments, a lipid nanoparticle has a mean diameter between about 10 and about 1000 nm. In some embodiments, a lipid nanoparticle has a diameter that is less than 300 nm. In some embodiments, a lipid nanoparticle has a diameter between about 10 and about 300 nm. In some embodiments, a lipid nanoparticle has a diameter that is less than 200 nm. In some embodiments, a lipid nanoparticle has a diameter between about 25 and about 200 nm. In some other embodiments, the lipid particles comprising a therapeutic nucleic acid and/or an immunosuppressant typically have a mean diameter of from about 20 nm to about 100 nm, 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm to ensure effective delivery. Nucleic acid containing lipid particles and their method of preparation are disclosed in, e.g., PCT/US18/50042, U.S. Patent Publication Nos. 20040142025 and 20070042031, the disclosures of which are herein incorporated by reference in their entirety for all purposes. In some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

According to some embodiments, a liquid pharmaceutical composition comprising a nucleic acid (e.g., a therapeutic nucleic acid, a nucleic acid used for research purposes) and/or inhibitor of the immune response (e.g., innate immune response) of the present invention may be formulated in lipid particles. In some embodiments, the lipid particle comprising a nucleic acid can be formed from a cationic lipid. In some other embodiments, the lipid particle comprising a nucleic acid can be formed from non-cationic lipid. In a preferred embodiment, the lipid particle of the invention is a nucleic acid containing lipid particle, which is formed from a cationic lipid comprising a nucleic acid selected from the group consisting of mRNA, antisense RNA and oligonucleotide, ribozymes, aptamer, interfering RNAs (RNAi), Dicer-substrate dsRNA, small hairpin RNA (shRNA), asymmetrical interfering RNA (aiRNA), microRNA (miRNA), minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone (dbDNA™) DNA vectors, minimalistic immunological-defined gene expression (MIDGE)-vector, nonviral ministring DNA vector (linear-covalently closed DNA vector), or dumbbell-shaped DNA minimal vector (“dumbbell DNA”).

Various lipid nanoparticles known in the art can be used to deliver a closed-ended DNA vector, including a ceDNA vector as described herein. For example, various delivery methods using lipid nanoparticles are described in U.S. Pat. Nos. 9,404,127, 9,006,417 and 9,518,272.

In some embodiments, a closed-ended DNA vector, including a ceDNA vector, and one or more inhibitors of the immune response (e.g., the innate immune response) as described herein, is delivered by a gold nanoparticle. Generally, a nucleic acid can be covalently bound to a gold nanoparticle or non-covalently bound to a gold nanoparticle (e.g., bound by a charge-charge interaction), for example as described by Ding et al. (2014). Gold Nanoparticles for Nucleic Acid Delivery. Mol. Ther. 22(6); 1075-1083. In some embodiments, gold nanoparticle-nucleic acid conjugates are produced using methods described, for example, in U.S. Pat. No. 6,812,334.

### C. Conjugates

In some embodiments, a closed-ended DNA vector, including a ceDNA vector, and one or more inhibitors of the immune response (e.g., the innate immune response) as described herein, as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

In some embodiments, a closed-ended DNA vector, including a ceDNA vector, and one or more inhibitors of the immune response (e.g., the innate immune response) as described herein, as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. In some embodiments, a ceDNA vector as disclosed herein is conjugated to a poly(amide) polymer, for example as described by U.S. Pat. No. 8,987,377. In some embodiments, a nucleic acid described by the disclosure is conjugated to a folic acid molecule as described in U.S. Pat. No. 8,507,455.

In some embodiments, a closed-ended DNA vector, including a ceDNA vector, and rapamycin or a rapamycin analogue as described herein, as disclosed herein is conjugated to a carbohydrate, for example as described in U.S. Pat. No. 8,450,467.

In some embodiments, the lipid nanoparticles may be conjugated with other moieties to prevent aggregation. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates; see, e.g., U.S. Provisional Application No. 61/294,828, filed Jan. 13, 2010, and U.S. Provisional Application No. 61/295,140, filed Jan. 14, 2010), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain preferred embodiments, non-ester containing linker moieties, such as amides or carbamates, are used. The disclosures of each of the above patent documents are herein incorporated by reference in their entirety for all purposes.

### D. Nanocapsule

Alternatively, nanocapsule formulations of a closed-ended DNA vector, including a ceDNA vector, and rapamycin or a rapamycin analogue as described herein, as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### E. Liposomes

A closed-ended DNA vector, including a ceDNA vector, and one or more inhibitors of the immune response (e.g., the innate immune response) as described herein, can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes are generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

F. Exemplary liposome and Lipid Nanoparticle (LNP) Compositions

A closed-ended DNA vector, including a ceDNA vector, and one or more inhibitors of the immune response (e.g., the innate immune response) as described herein, can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA are disclosed in International Application PCT/US2018/050042, filed on Sep. 7, 2018, and International Application PCT/US2018/064242, filed on Dec. 6, 2018, which are each incorporated herein by reference in their entirety and envisioned for use in the methods and compositions as disclosed herein.

In some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

In some aspects, the disclosure provides for a liposome formulation that will deliver an API with extended release or controlled release profile over a period of hours to weeks. In some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

In some aspects, the liposome formulation comprises sphingomyelin and one or more lipids disclosed herein. In some aspects, the liposome formulation comprises optisomes.

In some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

In some aspects, the disclosure provides for a liposome formulation comprising phospholipid, cholesterol and a PEG-ylated lipid in a molar ratio of 56:38:5. In some aspects, the liposome formulation's overall lipid content is from 2-16 mg/mL. In some aspects, the disclosure provides for a liposome formulation comprising a lipid containing a phosphatidylcholine functional group, a lipid containing an ethanolamine functional group and a PEG-ylated lipid. In some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. In some aspects, the disclosure provides for a liposome formulation comprising a lipid containing a phosphatidylcholine functional group, cholesterol and a PEG-ylated lipid. In some aspects, the disclosure provides for a liposome formulation comprising a lipid containing a phosphatidylcholine functional group and cholesterol. In some aspects, the PEG-ylated lipid is PEG-2000-DSPE. In some aspects, the disclosure provides for a liposome formulation comprising DPPG, soy PC, MPEG-DSPE lipid conjugate and cholesterol.

In some aspects, the disclosure provides for a liposome formulation comprising one or more lipids containing a phosphatidylcholine functional group and one or more lipids containing an ethanolamine functional group. In some aspects, the disclosure provides for a liposome formulation comprising one or more: lipids containing a phosphatidylcholine functional group, lipids containing an ethanolamine functional group, and sterols, e.g. cholesterol. In some aspects, the liposome formulation comprises DOPC/DEPC; and DOPE.

In some aspects, the disclosure provides for a liposome formulation further comprising one or more pharmaceutical excipients, e.g. sucrose and/or glycine.

In some aspects, the disclosure provides for a liposome formulation that is either unilamellar or multilamellar in structure. In some aspects, the disclosure provides for a liposome formulation that comprises multi-vesicular particles and/or foam-based particles. In some aspects, the disclosure provides for a liposome formulation that are larger in relative size to common nanoparticles and about 150 to 250 nm in size. In some aspects, the liposome formulation is a lyophilized powder.

In some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. In some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g. polyphosphate or sucrose octasulfate.

In some aspects, the disclosure provides for a lipid nanoparticle comprising a DNA vector, including a ceDNA vector as described herein and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid particles are prepared at a total lipid to ceDNA (mass or weight) ratio of from about 10:1 to 30:1. In some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. According to some embodiments of any of the aspects or embodiments herein, the composition has a total lipid to ceDNA ratio of about 15:1. According to some embodiments of any of the aspects or embodiments herein, the composition has a total lipid to ceDNA ratio of about 30:1. According to some embodiments of any of the aspects or embodiments herein, the composition has a total lipid to ceDNA ratio of about 40:1. According to some embodiments of any of the aspects or embodiments herein, the composition has a total lipid to ceDNA ratio of about 50:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

In some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

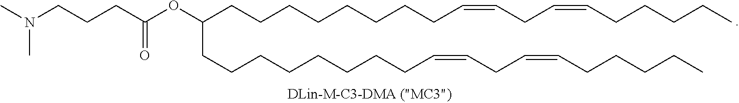 [SMILES: CCCCC/C=C\C/C=C\CCCCCCCCC(CCCCCCCC/C=C\C/C=C\CCCCC)OC(=O)CCCN(C)C]

The lipid DLin-MC3-DMA is described in Jayaraman et al., Angew. Chem. Int. Ed Engl. (2012), 51(34): 8529-8533, content of which is incorporated herein by reference in its entirety.

In some embodiments, the ionizable lipid is the lipid ATX-002 as described in WO2015/074085, content of which is incorporated herein by reference in its entirety.

In some embodiments, the ionizable lipid is (13Z,16Z)-N,N-dimethyl-3-nonyldocosa-13,16-dien-1-amine (Compound 32), as described in WO2012/040184, content of which is incorporated herein by reference in its entirety.

In some embodiments, the ionizable lipid is Compound 6 or Compound 22 as described in WO2015/199952, content of which is incorporated herein by reference in its entirety.

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. In some embodiments, ionizable lipid comprises from about 50 mol % to about 90 mol % of the total lipid present in the lipid nanoparticle.

In some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions comprising a DNA vector, including a ceDNA vector as described herein are described in International Application PCT/US2018/050042, filed on Sep. 7, 2018, and PCT/US2018/064242, filed on Dec. 6, 2018 which is incorporated herein in its entirety.

Exemplary non-cationic lipids are described in International application Publication WO2017/099823 and US patent publication U52018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

In some embodiments, the lipid nanoparticles do not comprise any phospholipids. In some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication U52010/0130588, contents of both of which are incorporated herein by reference in their entirety.

The component providing membrane integrity, such as a sterol, can comprise 0-50% (mol) of the total lipid present in the lipid nanoparticle. In some embodiments, such a component is 20-50% (mol) 30-40% (mol) of the total lipid content of the lipid nanoparticle.

In some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a PEGylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

In some embodiments, a PEG-lipid is a compound disclosed in US2018/0028664, the content of which is incorporated herein by reference in its entirety.

In some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some examples, the PEG-lipid can be selected from the group consisting of PEG-DMG, 1,2-dimyristoyl-sn-glycero-3-phosphoethanolamine-N-[methoxy(polyethylene glycol)-2000].

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

In some embodiments, the one or more additional compound can be a therapeutic agent. The therapeutic agent can be selected from any class suitable for the therapeutic objective. In other words, the therapeutic agent can be selected from any class suitable for the therapeutic objective. In other words, the therapeutic agent can be selected according to the treatment objective and biological action desired. For example, if the ceDNA within the LNP is useful for treating cancer, the additional compound can be an anti-cancer agent (e.g., a chemotherapeutic agent, a targeted cancer therapy (including, but not limited to, a small molecule, an antibody, or an antibody-drug conjugate). In another example, if the LNP containing the ceDNA is useful for treating an infection, the additional compound can be an antimicrobial agent (e.g., an antibiotic or antiviral compound). In yet another example, if the LNP containing the ceDNA is useful for treating an immune disease or disorder, the additional compound can be a compound that modulates an immune response (e.g., an immunosuppressant, immunostimulatory compound, or compound modulating one or more specific immune pathways). In some embodiments, different cocktails of different lipid nanoparticles containing different compounds, such as a ceDNA encoding a different protein or a different compound, such as a therapeutic may be used in the compositions and methods of the invention.

In some embodiments, the additional compound is an immune modulating agent. For example, the additional compound is an immunosuppressant. In some embodiments, the additional compound is immune stimulatory agent.

Also provided herein is a pharmaceutical composition comprising the lipid nanoparticle-encapsulated ceDNA vector and rapamycin or rapamycin analogue as described herein and a pharmaceutically acceptable carrier or excipient. Also provided herein is a pharmaceutical composition comprising the lipid nanoparticle-encapsulated ceDNA vector and a pharmaceutically acceptable carrier or excipient, where the rapamycin or rapamycin analogue is co-administered to the subject in a different composition as described herein.

In some aspects, the disclosure provides for a lipid nanoparticle formulation further comprising one or more pharmaceutical excipients. In some embodiments, the lipid nanoparticle formulation further comprises sucrose, tris, trehalose and/or glycine.

A closed-ended DNA vector, including a ceDNA vector, and optionally one or more inhibitors of the immune response (e.g., the innate immune response) as described herein, can be complexed with the lipid portion of the particle or encapsulated in the lipid position of the lipid nanoparticle. In some embodiments, a DNA vector, including a ceDNA vector as described herein can be fully encapsulated in the lipid position of the lipid nanoparticle, thereby protecting it from degradation by a nuclease, e.g., in an aqueous solution. In some embodiments, a DNA vector, including a ceDNA vector as described herein in the lipid nanoparticle is not substantially degraded after exposure of the lipid nanoparticle to a nuclease at 37° C. for at least about 20, 30, 45, or 60 minutes. In some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. In some aspects, the lipid nanoparticle formulation is a lyophilized powder.

In some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

In some further embodiments, the lipid nanoparticles having a non-lamellar morphology are electron dense. In some aspects, the disclosure provides for a lipid nanoparticle that is either unilamellar or multilamellar in structure. In some aspects, the disclosure provides for a lipid nanoparticle formulation that comprises multi-vesicular particles and/or foam-based particles. By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size. The pKa of formulated cationic lipids can be correlated with the effectiveness of the LNPs for delivery of nucleic acids (see Jayaraman et al, Angewandte Chemie, International Edition (2012), 51(34), 8529-8533; Semple et al., Nature Biotechnology 28, 172-176 (20 1 0), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

### VI. Inhibitors of the Immune Response

Provided herein are inhibitors or the immune response. According to embodiments, the inhibitors of the immune response are inhibitors of the innate immune response.

### Rapamycin or Rapamycin Analogs

According to some aspects, the disclosure provides non-viral, capsid-free DNA vectors with covalently-closed ends (ceDNA vector) administered in conjunction with rapamycin or rapamycin analogs. In some embodiments, the rapamycin or rapamycin analog is present in a super-saturated amount in a synthetic nanocarrier as described in WO 2016/073799. In some embodiments, the ceDNA vector is also present in the same nanocarrier.

In some embodiments of the compositions and methods described herein, rapamycin or a rapamycin analog is co-administered with a ceDNA vector to a subject. In some embodiments of the compositions and methods described herein, the ceDNA vector and rapamycin or rapamycin analog are co-administered together in a single formulation. In some embodiments of the compositions and methods described herein, the rapamycin or rapamycin analog is present in a supersaturated concentration in a synthetic nanocarrier as described in WO 2016/073799. In some embodiments, the ceDNA vector is also present in the same nanocarrier. In some embodiments, the ceDNA vector formulated in a lipid nanoparticle is also present in the same nanocarrier.

In some embodiments, the rapamycin analog is any of the rapamycin analogs known in the art, such as any of the rapamycin analogs described in U.S. Pat. No. 5,138,051, or WO 2017/040341, the contents of each of which are herein incorporated by reference in their entireties.

In some embodiments, the rapamycin analog is a compound of Formula I as shown below:

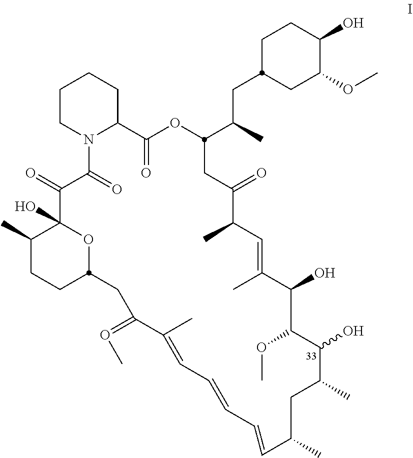

In some embodiments, the rapamycin analog is a compound of Formula II where the configuration of the substituents on C-33 of Formula I is the R configuration as shown below:

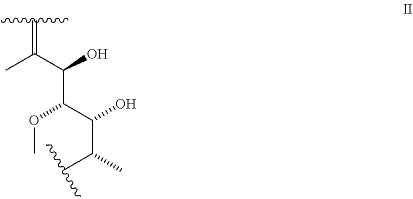 [SMILES: *[C@@H](C)[C@@H](O)[C@H](OC)[C@H](O)C(=*)C]

In some embodiments, the rapamycin analog is a compound of Formula III as shown below:

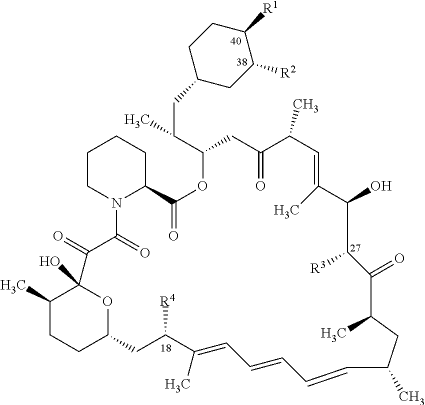

or a pharmaceutically acceptable salt thereof, wherein: R¹ is OH or OCH₃R² is H or F R³ is H, OH, or OCH₃; and R⁴ is OH or OCH₃.

[symbol] In some embodiments, the rapamycin analog is a compound of Formula III in pure [symbol] l form as a single diastereomer of Formula IV, as shown below:

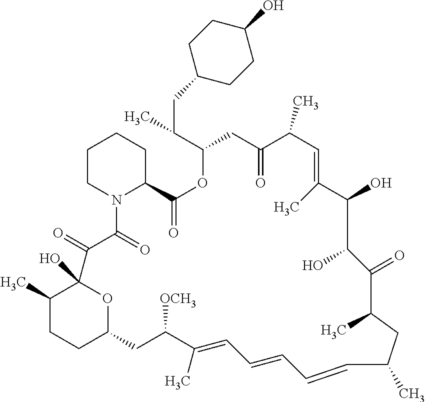

In some embodiments, the rapamycin analog is a compound of Formula III in pure chiral form as a single diastereomer of Formula V, as shown below:

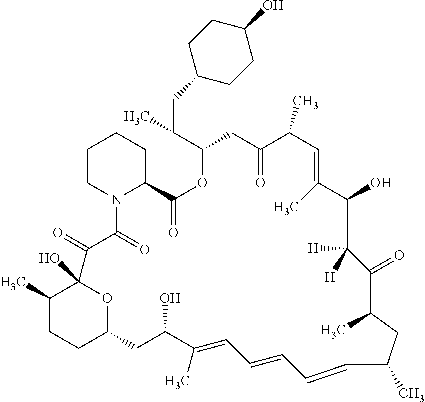

In some embodiments, the rapamycin analog is a compound of Formula III in pure chiral form as a single diastereomer of Formula VI, as shown below:

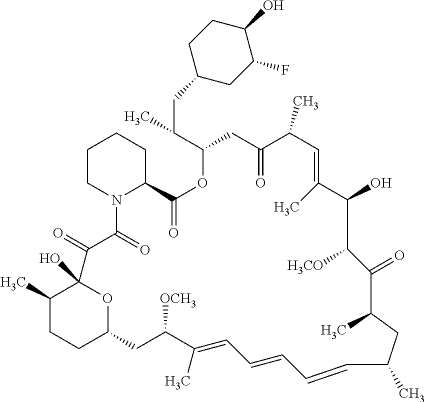

In some embodiments, the rapamycin analog is a compound of Formula III in pure chiral form as a single diastereomer of Formula VII, as shown below:

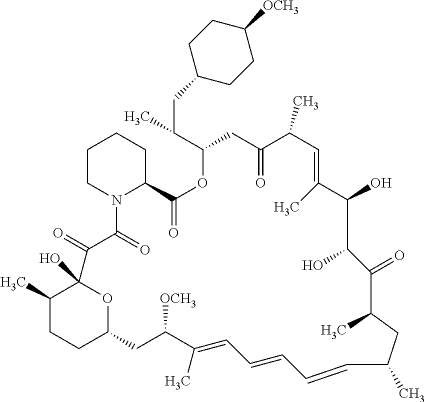

In some embodiments, the rapamycin analog is a compound of Formula III in pure chiral form as a single diastereomer of Formula VIII, as shown below:

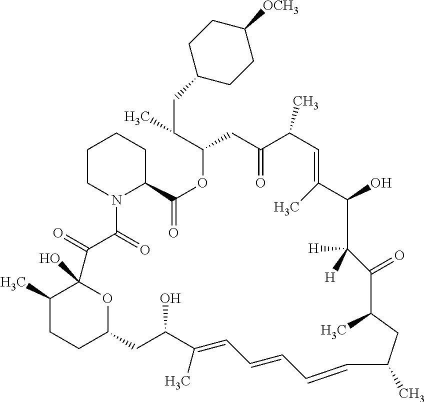

In some embodiments, the rapamycin analog is a compound of Formula IX, as shown below:

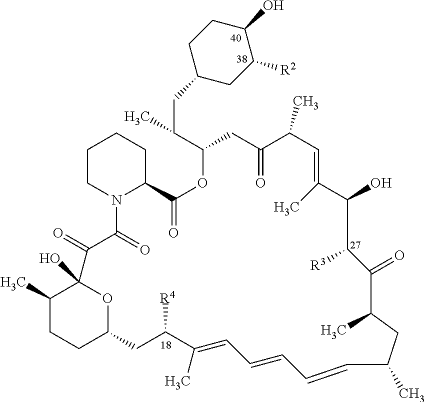

or a pharmaceutically acceptable salt thereof, where R² is H or F, R³ is OH, or OCH₃; and R⁴ is OCH₃ or OH. In certain embodiments R⁴ is OCH₃. In certain embodiments R4 is OCH₃, R2 is F, and R³ is OCH3. In certain embodiments R⁴ is OCH₃, R² is H, and R³ is OH. In certain embodiments R² is H, R₃ is H, and R⁴ is OH. In various embodiments the compounds of Formula IX are present as a racemic mixture.

Accordingly, in some embodiments, the rapamycin analog is selected from any one of Formulas I-IX or a derivative thereof.

In some embodiments, the rapamycin or rapamycin analog is delivered or administered using a synthetic nanocarrier as described in WO 2016/073799, incorporated by reference in its entirety herein.

As described in WO 2016/073799, the concentration of rapamycin in the formulation during synthetic nanocarrier formation, relative to the solubility limit of the rapamycin in said formulation, can have a significant impact on the ability of the resulting synthetic nanocarriers to induce immune tolerance. In addition, how such rapamycin is dispersed through the synthetic nanocarriers can impact whether or not the resulting synthetic nanocarriers are initially sterile filterable. Accordingly, in some embodiments, synthetic nanocarriers created under conditions that result in a concentration of rapamycin that exceeds its solubility in the formed nanocarrier suspension are used in the compositions and methods described herein. Such synthetic nanocarriers can provide for more durable immune tolerance and be initially sterile filterable.

In some embodiments, the ceDNA vector is co-administered with a composition comprising synthetic nanocarriers comprising a hydrophobic polyester carrier material and rapamycin or rapamycin analog, wherein the rapamycin or rapamycin analog is present in the synthetic nanocarriers in a stable, super-saturated amount that is less than 50 weight % based on the weight of rapamycin or rapamycin analog relative to the weight of hydrophobic polyester carrier material is provided.

In one embodiment of any one of the compositions or methods provided herein, the weights are the recipe weights of the materials that are combined during the formulation of the synthetic nanocarriers. In one embodiment of any one of the compositions or methods provided herein, the weights are the weights of the materials in the resulting synthetic nanocarrier composition.

In some embodiments of any one of the compositions and methods provided herein, the rapamycin or rapamycin analog is present in a stable, super-saturated amount that is less than 45 weight %. In one embodiment of any one of the compositions and methods provided herein, the rapamycin or rapamycin analog is present in a stable, super-saturated amount that is less than 40 weight %. In one embodiment of any one of the compositions and methods provided herein, the rapamycin or rapamycin analog is present in a stable, super-saturated amount that is less than 35 weight %. In one embodiment of any one of the compositions and methods provided herein, the rapamycin or rapamycin analog is present in a stable, super-saturated amount that is less than 30 weight %. In one embodiment of any one of the compositions and methods provided herein, the rapamycin or rapamycin analog is present in a stable, super-saturated amount that is less than 25 weight %. In one embodiment of any one of the compositions and methods provided herein, the rapamycin or rapamycin analog is present in a stable, super-saturated amount that is less than 20 weight %. In one embodiment of any one of the compositions and methods provided herein, the rapamycin or rapamycin analog is present in a stable, super-saturated amount that is less than 15 weight %. In one embodiment of any one of the compositions and methods provided herein, the rapamycin or rapamycin analog is present in a stable, super-saturated amount that is less than 10 weight %. In one embodiment of any one of the compositions and methods provided herein, the rapamycin or rapamycin analog is present in a stable, super-saturated amount that is greater than 7 weight %.

In one embodiment of any one of the compositions and methods provided herein, the hydrophobic polyester carrier material comprises PLA, PLG, PLGA or polycaprolactone. In one embodiment of any one of the compositions and methods provided herein, the hydrophobic polyester carrier material further comprises PLA-PEG, PLGA-PEG or PCL-PEG.

In one embodiment of any one of the compositions and methods provided herein, the amount of the hydrophobic polyester carrier material in the synthetic nanocarriers is 5-95 weight % hydrophobic polyester carrier material/total solids. In one embodiment of any one of the compositions and methods provided herein, the amount of hydrophobic polyester carrier material in the synthetic nanocarriers is 60-95 weight % hydrophobic polyester carrier material/total solids.

In one embodiment of any one of the compositions and methods provided herein, the synthetic nanocarriers further comprise a non-ionic surfactant with HLB value less than or equal to 10. In one embodiment of any one of the compositions and methods provided herein, the non-ionic surfactant with HLB value less than or equal to 10 comprises a sorbitan ester, fatty alcohol, fatty acid ester, ethoxylated fatty alcohol, poloxamer, fatty acid, cholesterol, cholesterol derivative, or bile acid or salt. In one embodiment of any one of the compositions and methods provided herein, the non-ionic surfactant with HLB value less than or equal to 10 comprises SPAN 40, SPAN 20, oleyl alcohol, stearyl alcohol, isopropyl palmitate, glycerol monostearate, BRIJ 52, BRIJ 93, Pluronic P-123, Pluronic L-31, palmitic acid, dodecanoic acid, glyceryl tripalmitate or glyceryl trilinoleate. In one embodiment of any one of the compositions and methods provided herein, the non-ionic surfactant with HLB value less than or equal to 10 is SPAN 40.

In one embodiment of any one of the compositions and methods provided herein, the non-ionic surfactant with HLB value less than or equal to 10 is encapsulated in the synthetic nanocarriers, present on the surface of the synthetic nanocarriers, or both. In one embodiment of any one of the compositions and methods provided herein, the amount of non-ionic surfactant with HLB value less than or equal to 10 is >0.1 but <15 weight % non-ionic surfactant with a HLB value less than or equal to 10/hydrophobic polyester carrier material. In one embodiment of any one of the compositions and methods provided herein, the amount of non-ionic surfactant with HLB value less than or equal to 10 is >1 but <13 weight % non-ionic surfactant with an HLB value less than or equal to 10/hydrophobic polyester carrier material. In one embodiment of any one of the compositions and methods provided herein, the amount of non-ionic surfactant with HLB value less than or equal to 10 is >1 but <9 weight % non-ionic surfactant with an HLB value less than or equal to 10/hydrophobic polyester carrier material.

In one embodiment of any one of the compositions and methods provided herein, the composition is initially sterile filterable through a 0.22μιη filter.

In one embodiment of any one of the compositions and methods provided herein, the mean of a particle size distribution obtained using dynamic light scattering of the synthetic nanocarriers is a diameter greater than 120 nm. In one embodiment of any one of the compositions and methods provided herein, the diameter is greater than 150 nm. In one embodiment of any one of the compositions and methods provided herein, the diameter is greater than 200 nm. In one embodiment of any one of the compositions and methods provided herein, the diameter is greater than 250 nm. In one embodiment of any one of the compositions and methods provided herein, the diameter is less than 300 nm. In one embodiment of any one of the compositions and methods provided herein, the diameter is less than 250 nm. In one embodiment of any one of the compositions and methods provided herein, the diameter is less than 200 nm.

In one embodiment of any one of the compositions and methods provided herein, the rapamycin or rapamycin analog is encapsulated in the synthetic nanocarriers.

In one embodiment of any one of the compositions and methods provided herein, the composition further comprises a pharmaceutically acceptable carrier.

In one embodiment of any one of the compositions or methods provided herein, the rapamycin or rapamycin analog is present in a super-saturated amount that is at least 1% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 5% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 10% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 15% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 20% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 25% over the saturation limit of the rapamycin or rapamycin analog in the hydrophobic polyester carrier material. In one embodiment of any one of the compositions or methods provided herein, the rapamycin or rapamycin analog is present in a super-saturated amount that is at least 30% over the saturation limit of the rapamycin or rapamycin analog in the hydrophobic polyester carrier material.

In another embodiment of any one of the compositions or methods provided herein, the amount of rapamycin or rapamycin analog exceeds the saturation limit by at least 1%. In another embodiment, the amount of rapamycin or rapamycin analog exceeds the saturation limit by at least 5%. In another embodiment, the amount of rapamycin or rapamycin analog exceeds the saturation limit by at least 10%. In another embodiment, the amount of rapamycin or rapamycin analog exceeds the saturation limit by at least 15%. In another embodiment, the amount of rapamycin or rapamycin analog exceeds the saturation limit by at least 20%. In another embodiment, the amount of rapamycin or rapamycin analog exceeds the saturation limit by at least 25%. In another embodiment, the amount of rapamycin or rapamycin analog exceeds the saturation limit by at least 30%.

Inhibitors of cGAS

According to some aspects, the disclosure provides non-viral, capsid-free DNA vectors with covalently-closed ends (ceDNA) administered in conjunction with one or more cGAS antagonists. Also provided herein are ceDNA constructs comprising sequences encoding, in part, one or more cGAS inhibitory RNAs or proteins.

cGAS is another class of PRRs triggered by cytosolic DNA, which binds to DNA and activates the ER-bound stimulator of interferon genes (STING). This results in activation of the type I interferon response and, in some cases, activation of other proposed cytosolic DNA sensors including Absent in Melanoma (AIM2), IFN-γ-inducible protein 16 (IFI16), Interferon-Inducible Protein X (IFIX), LRRFIP1, DHX9, DHX36, DDX41, Ku70, DNA-PKcs, MRN complex (including MRE11, Rad50 and Nbs1) and RNA polymerase III. AIM2, IFI16, and IFIX are pyrin and HIN200 domain proteins (PYHIN) proteins. Furthermore, it has been shown that unpaired DNA nucleotides flanking short base-paired DNA stretches, as in stem-loop structures of single-stranded DNA (ssDNA) derived from human immunodeficiency virus type 1 (HIV-1), activated the type I interferon-inducing DNA sensor cGAS in a sequence-dependent manner. DNA structures containing unpaired guanosines flanking short (12- to 20-bp) dsDNA (Y-form DNA) were highly stimulatory and specifically enhanced the enzymatic activity of cGAS

cGAS directly binds DNA by interactions with the sugar-phosphate backbone of both DNA strands (S. R. Paluden. Microbiology and Molecular Biology Reviews. 2015. 79(2): 225). This causes a conformational change in the enzyme allowing the nucleotide substrates ATP and GTP to access the active site, resulting in cGAMP synthesis (A. Dempsey and A. G. Bowie, Virology 2015 May, 0: 146-152). cGAMP then binds STING, thus leading to Type I interferon production (A. Dempsey and A. G. Bowie, Virology 2015 May, 0: 146-152). Importantly, cGAS contacts dsDNA solely through the DNA phosphate backbone, leading to nucleotide sequence-independent sensing (A. Dempsey and A. G. Bowie, Virology 2015 May, 0: 146-152). It has also been shown that cGAS can be activated by unpaired DNA nucleotides, specifically guanosines, flanking short base-paired DNA stretches of 12-20 bp, as in stem-loop structures of single-stranded DNA (ssDNA) derived from human immunodeficiency virus type 1 (HIV-1) (M. H. Christnesen and S. R. Paluden. Cellular and Molecular Immunology. 2017. 14:4-13; A-M Herzner et al., 2015. Nature Immunology).

Accordingly, structural features of ceDNAs important for innate immune activation by PRRs include, but are not limited to, the modified AAV inverted terminal repeat sequences (ITRs), including the Rep-binding site (RBS) and terminal resolution site (TRS); the hairpin sequences in the ITR; the CG rich nature of the RBS; the absence of DNA methylation; and linear duplex DNA structure with flanking ITRs that can have e.g. single-stranded looped DNA.

In some embodiments of the compositions and methods described herein, an inhibitor of cGAS is co-administered with a ceDNA to a subject. In some embodiments of the compositions and methods described herein, where the inhibitor of cGAS is an RNA or protein sequence, the ceDNA encodes the RNA or protein inhibitor of cGAS.

In some embodiments, the inhibitor of cGAS is an antimalarial drug (J. An et al., J. Immunol. Mar. 27, 2015). In some embodiments, the antimalarial drug is an aminoquinoline-based or aminoacridine-based antimalarial drug (J. An et al., J. Immunol. Mar. 27, 2015). In some embodiments, the antimalarial drug is selected from quinacrine (QC), 9-amino-6-chloro-2-methoxyacridine (AMCA), hydroxychloroquine (HCQ), and chloroquine (CQ) (J. An et al., J. Immunol. Mar. 27, 2015).

In some embodiments, the inhibitor of cGAS is a small molecule compound that binds to the catalytic pocket of cGAS (J. Vincent et al., Nature Communications, 8:750). In some embodiments, the small molecule compound that binds to the catalytic pocket of cGAS is selected from RU166365, RU281332, RU320521, RU320519, RU320461, RU320462, RU320520, RU320467, and RU320582 (J. Vincent et al., Nature Communications, 8:750). In some embodiments, the small molecule compound that binds to the catalytic pocket of cGAS is RU320521 (J. Vincent et al., Nature Communications, 8:750). In some embodiments, the small molecule compound that binds to the catalytic pocket of cGAS is selected from compound 15, compound 16, compound 17, compound 18, compound 19, and PF-06928215 (J. Vincent et al., Nature Communications, 8:750; PLOS ONE. Sep. 21, 2017). In some embodiments, the small molecule compound that binds to the catalytic pocket of cGAS is PF-06928215 (PLOS ONE. Sep. 21, 2017)

In some embodiments, the inhibitor of cGAS is any of the small molecule compounds described in U520160068560, the contents of which are herein incorporated by reference in their entireties.

In some embodiments of the compositions and methods described herein, an inhibitor of cGAS is encoded by a ceDNA being administered to a subject (including, e.g. subsequent delivery of ceDNA). In some embodiments of the compositions and methods described herein, the inhibitor of cGAS encoded by a ceDNA being administered to a subject is Kaposi's sarcoma-associated herpesvirus protein ORF52 having an amino acid sequence of MAAPRGRPKKDLTMEDLTAKISQLTVENRELRKALGSTADPRDRPLTATEKEAQLTATVGA LSAAAAKKIEARVRTIFSKVVTQKQVDDALKGLSLRIDVCMSDGGTAKPPPGANNRRRRGAS TTRAGVDD (SEQ ID NO: 882) or a variant thereof that inhibits cGAS (M. H. Christnesen and S. R. Paluden. Cellular and Molecular Immunology. 2017. 14:4-13). In some embodiments of the compositions and methods described herein, the inhibitor of cGAS encoded by a ceDNA being administered to a subject is a gammaherpesvirus ortholog of ORF52.

In some embodiments of the compositions and methods described herein, the inhibitor of cGAS encoded by a ceDNA being administered to a subject is a cytoplasmic isoform of Kaposi sarcoma herpresvirus LANA (latency-associated nuclear antigen), also referred to herein, as a “cytoplasmic LANA isoform,” or a variant thereof that inhibits cGAS (Zhang G. et al., Proc Natl Acad Sci USA. 2016 Feb. 23; 113 (8):E1034-43). LANA or ORF73 has a sequence of the following 1129 amino acids:

(SEQ ID NO: 883)
MAPPGMRLRSGRSTGAPLTRGSCRKRNRSPERCDLGDDLHLQPRRKHVA
DSVDGRECGPHTLPIPGSPTVFTSGLPAFVSSPTLPVAPIPSPAPATPL
PPPALLPPVTTSSSPIPPSHPVSPGTTDTHSPSPALPPTQSPESSQRPP
LSSPTGRPDSSTPMRPPPSQQTTPPHSPTTPPPEPPSKSSPDSLAPSTL
RSLRKRRLSSPQGPSTLNPICQSPPVSPPRCDFANRSVYPPWATESPIY
VGSSSDGDTPPRQPPTSPISIGSSSPSEGSWGDDTAMLVLLAEIAEEAS
KNEKECSENNQAGEDNGDNEISKESQVDKDDNDNKDDEEEQETDEEDEE
DDEEDDEEDDEEDDEEDDEEDDEEDDEEEDEEEDEEEDEEEDEEEEEDE
EDDDDEDNEDEEDDEEEDKKEDEEDGGDGNKTLSIQSSQQQQEPQQQEP
QQQEPQQQEPQQQEPQQQEPQQQEPQQQEPQQREPQQREPQQREPQQRE
PQQREPQQREPQQREPQQREPQQREPQQREPQQREPQQREPQQQEPQQQ
EPQQQEPQQQEPQQQEPQQQEPQQQEPQQQEPQQQEPQQQEPQQQEPQQ
QEPQQQDEQQQDEQQQDEQQQDEQQQDEQQQDEQQQDEQQQDEQEQQDE
QQQDEQQQQDEQEQQEEQEQQEEQQQDEQQQDEQQQDEQQQDEQEQQDE
QQQDEQQQQDEQEQQEEQEQQEEQEQQEEQEQQEEQEQELEEQEQELEE
QEQELEEQEQELEEQEQELEEQEQELEEQEQELEEQEQELEEQEQELEE
QEQELEEQEQELEEQEQELEEQEQELEEQEQELEEQEQEQELEEVEEQE
QEQEEQELEEVEEQEQEQEEQEEQELEEVEEQEEQELEEVEEQEEQELE
EVEEQEQQGVEQQEQETVEEPIILHGSSSEDEMEVDYPVVSTHEQIASS
PPGDNTPDDDPQPGPSREYRYVLRTSPPHRPGVRMRRVPVTHPKKPHPR
YQQPPVPYRQIDDCPAKARPQHIFYRRFLGKDGRRDPKCQWKFAVIFWG
NDPYGLKKLSQAFQFGGVKAGPVSCLPHPGPDQSPITYCVYVYCQNKDT
SKKVQMARLAWEASHPLAGNLQSSIVKFKKPLPLTQPGENQGPGDSPQE
MT.

A non-limiting example of a truncated cytoplasmic LANA isoform for use with the ceDNAs described herein is LANAΔ161 or SEQ ID NO: 532 (lacking amino acids 161-1162 of SEQ ID NO: 884).

In some embodiments of the compositions and methods described herein, an inhibitor of cGAS is an antibody or antigen-binding fragment that binds cGAS. In some embodiments of the compositions and methods described herein, the antibody or antigen-binding fragment that binds cGAS is encoded by the ceDNA.

In some embodiments of the compositions and methods described herein, an inhibitor of cGAS is an RNA inhibitor of cGAS, such as an siRNA specific for cGAS. In some embodiments of the compositions and methods described herein, the RNA inhibitor of cGAS is encoded by the ceDNA.

In some embodiments of the compositions and methods described herein, an inhibitor of cGAS is miRNA inhibitor of cGAS, such as miR-25 (GGCCAGTGTTGAGAGGCGGAGACTTGGGCAATTGCTGGACGCTGCCCTGGGCATTGCAC TTGTCTCGGTCTGACAGTGCCGGCC; SEQ ID NO: 885) and miR-93 (CTGGGGGCTCCAAAGTGCTGTTCGTGCAGGTAGTGTGATTACCCAACCTACTGCTGAGC TAGCACTTCCCGAGCCCCCGG; SEQ ID NO: 886)¹¹. miR-25 and miR-93 are thought to target nuclear receptor coactivator 3 (NCOA3), an epigenetic factor that maintains basal levels of cGAS expression leading to repression of cGAS (Wu et al. 2017. Nat. Cell Biot 19(10):1286-1296). In some embodiments of the compositions and methods described herein, the miRNA inhibitor of cGAS is encoded by the ceDNA.

### Inhibitors of TLR

According to some aspects, the disclosure provides non-viral, capsid-free DNA vectors with covalently-closed ends (ceDNA) administered in conjunction with one or more TLR antagonists. Also provided herein are ceDNA constructs comprising sequences encoding, in part, one or more TLR inhibitory oligonucleotides. According to some aspects, the disclosure provides non-viral, capsid-free DNA vectors with covalently-closed ends (ceDNA) administered in conjunction with one or more TLR9 antagonists. Also provided herein are ceDNA constructs comprising sequences encoding, in part, one or more TLR9 inhibitory oligonucleotides.

According to some embodiments, the TLR9 inhibitor is a small molecule antagonist. In another embodiment, the TLR9 inhibitor is an antibody against TLR9. According to some embodiments, the TLR9 antibody is a monoclonal antibody. In some embodiments of the compositions and methods described herein, one or more terminal structural elements of a ceDNA, such as the ITR sequences, comprise a sequence of a TLR9 inhibitory oligonucleotide.

In some embodiments of the compositions and methods described herein, a TLR9 inhibitory oligonucleotide has one or more of the following features (i) three consecutive G nucleotides at the 3′ end; (ii) a CC(T) triplet at the 5′ end; and (iii) a distance between the 5′ CC(T) and downstream GGG triplet optimally 3-5 nucleotides long. In some embodiments, the TLR9 inhibitory oligonucleotide has a sequence of 5′CCTN(3-5)G(3-5)RR3′ (SEQ ID NO: 887). In some embodiments, the TLR9 inhibitory oligonucleotide does not have intrachain and/or interchain Hoogsten hydrogen bonding between adjacent Gs.

In some embodiments of the compositions and methods described herein, the TLR9 inhibitory oligonucleotide is a Class G TLR9 inhibitory oligonucleotide having G4 stacking characteristics, and comprise multiple G3 triplets or G4 tetrads, such as an inhibitory oligonucleotide comprising TTAGGGn (SEQ ID NO: 888). Non-limiting examples of such Class G TLR9 inhibitory oligonucleotide include ODN-2088 (TCCTGGCGGGGAAGT, SEQ ID NO: 889), ODN-2114 (TCCTGGAGGGGAAGT, SEQ ID NO: 890), poly-G (GGGGGGGGGGGGGGGGGGGG, SEQ ID NO: 891), ODN-A151 (TTAGGGTTAGGGTTAGGGTTAGGG, SEQ ID NO: 892), G-ODN (CTCCTATTGGGGGTTTCCTAT, SEQ ID NO: 893), and IRS-869 (TCCTGGAGGGGTTGT, SEQ ID NO: 894) and AS1411 (GGTGGTGGTGGTTGTGGTGGTGGTGG, SEQ ID NO: 903).

In some embodiments of the compositions and methods described herein, the TLR9 inhibitory oligonucleotide is a Class R TLR9 inhibitory oligonucleotide having characteristics including being palindromic and/or having short 5′ or 3′ overhangs, such as an INH-1 inhibitory oligonucleotide. Non-limiting examples of such Class R TLR9 inhibitory oligonucleotide include

INH-1
(CCTGGATGGGAATTCCCATCCAGG, SEQ ID NO: 895),
INH-4
(TTCCCATCCAGGCCTGGATGGGAA, SEQ ID NO: 896),
and
IRS-661
(TGCTTGCAAGCTTGCAAGCA, SEQ ID NO: 897).

In some embodiments of the compositions and methods described herein, the TLR9 inhibitory oligonucleotide is a Class B TLR9 inhibitory oligonucleotide having linear characteristics and a 5′ CC(T)→GGG-3′ motif, such as an INH-18 inhibitory oligonucleotide. Non-limiting examples of such Class B TLR9 inhibitory oligonucleotide include

ODN-2088
(TCCTGGCGGGGAAGT, SEQ ID NO: 889),
ODN-2114
(TCCTGGAGGGGAAGT, SEQ ID NO: 890),
4024
(TCCTGGATGGGAAGT, SEQ ID NO: 898),
4084F
(CCTGGATGGGAA, SEQ ID NO: 899),
INH-13
(CTTACCGCTGCACCTGGATGGGAA, SEQ ID NO: 900),
INH-18
(CCTGGATGGGAACTTACCGCTGCA, SEQ ID NO: 901),
G-ODN
(CTCCTATTGGGGGTTTCCTAT, SEQ ID NO: 893),
IRS-869
(TCCTGGAGGGGTTGT, SEQ ID NO: 864),
IRS-954
TGCTCCTGGAGGGGTTGT, SEQ ID NO: 902),
and
AS1411
(GGTGGTGGTGGTTGTGGTGGTGGTGG, SEQ ID NO: 903).

In some embodiments of the compositions and methods described herein, a coding sequence encoded by a ceDNA, such as the transgene sequence, is modified so that CpG di-nucleotides allocated within a codon triplet for a selected amino acid are changed to a codon triplet for the same amino acid lacking a CpG di-nucleotide.

In some embodiments of the compositions and methods described herein, where the inhibitor of TLR9 is an RNA or protein sequence, the ceDNA encodes the RNA or protein inhibitor of TLR9. In some embodiments of the compositions and methods described herein, an inhibitor of TLR9 is an antibody or antigen-binding fragment that binds TLR9. In some embodiments of the compositions and methods described herein, the antibody or antigen-binding fragment that binds TLR9 is encoded by the ceDNA.

In some embodiments of the compositions and methods described herein, an inhibitor of TLR9 is co-administered with a ceDNA to a subject. Non-limiting examples of inhibitors of TLR9 can be found in “Classification, Mechanisms of Action, and Therapeutic Applications of Inhibitory Oligonucleotides for Toll-Like Receptors (TLR) 7 and 9,” P. S. Lenert, Mediators of Inflammation, Vol. 2010, 986596; U520150203850; and U52017026800, the contents of each of which are herein incorporated by reference in their entireties.

Accordingly, in some embodiments of the compositions and methods described herein, an inhibitor of TLR9 is co-administered with a ceDNA to a subject.

In some embodiments of the compositions and methods described herein, an inhibitor of TLR9 is encoded in cis by a ceDNA being administered to a subject (including, e.g. subsequent delivery of ceDNA). In some embodiments of the compositions and methods described herein, an inhibitor of TLR9 is administered in trans by a ceDNA being administered to a subject.

In some embodiments of the compositions and methods described herein, an inhibitor of TLR9 is a TLR9 inhibitory oligonucleotide

In some embodiments of the compositions and methods described herein, a TLR9 inhibitory oligonucleotide has one or more of the following features (i) three consecutive G nucleotides at the 3′ end; (ii) a CC(T) triplet at the 5′ end; and (iii) a distance between the 5′ CC(T) and downstream GGG triplet is optimally between 3-5 nucleotides long. In some embodiments, the TLR9 inhibitory oligonucleotide has a sequence of 5′CCTN(3-5)G(3-5)RR3′ (SEQ ID NO: 887). In some embodiments, the TLR9 inhibitory oligonucleotide does not have intrachain and/or interchain Hoogsten hydrogen bonding between adjacent Gs.

In some embodiments of the compositions and methods described herein, an inhibitor of TLR9 is an antibody or antigen-binding fragment that binds TLR9. In some embodiments of the compositions and methods described herein, the antibody or antigen-binding fragment that binds TLR9 is encoded by the ceDNA.

In some embodiments of the compositions and methods described herein, an inhibitor of TLR9 is an inhibitor of endosomal acidification, e.g., chloroquine.

### Inflammasome Antagonists

### Inhibitors of the NLRP3 Inflammasome Pathway:

In some embodiments, an inflammasome antagonist inhibits NLRP3. The term “NLRP3” is also referred to as Cryopyrin refers to NOD-like receptor family, pyrin domain containing 3) inflammasome or NACHT, LRR and PYD domains-containing protein 3 (NALP3), also known as cryopyrin, cold induced autoinflammatory syndrome 1 (CIAS1), caterpillar-like receptor 1.1 (CLR1.1) or Pyrin Domain-Containing Apafl-Like Protein 1 (PYPAF1). NALP3 is also known by aliases: NLRP3 PYD-NACHT-NAD-LRR NALP3 Cias1, Pypaf1, Mmig1 PYD-NACHT-NAD-LRR). NLRP3 is a component of a multiprotein oligomer consisting of the NLRP3 protein, ASC (apoptosis-associated speck-like protein containing a CARD) and pro-caspase 1.

NLRP3 inhibitors encompassed for use in the methods and compositions herein are disclosed in Shao, Bo-Zong, et al. “NLRP3 inflammasome and its inhibitors: a review.” Frontiers in pharmacology 6 (2015): 262., and Wang et al., Lab investigation, 2017, 97; 922-934, which are incorporated herein in their entirety by reference.

In some embodiments, an inhibitor of the NLRP3 inflammasome is MCC950 or a functional derivative hereof. MCC950 has the formula:

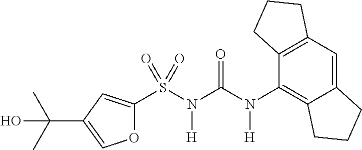 [SMILES: CC(C)(O)c1coc(S(=O)(=O)NC(=O)Nc2c3c(cc4c2CCC4)CCC3)c1]

and is a potent and selective inhibitor of the NLRP3. MCC950 blocks the release of IL-1β induced by NLRP3 activators, such as ATP, MSU and nigericin, by preventing oligomerization of the inflammasome adaptor protein ASC (apoptosis-associated speck-like protein containing a CARD) (Coll R C. et al., 2015. A small-molecule inhibitor of the NLRP3 inflammasome for the treatment of inflammatory diseases. Nature Med 21(3), 248-255; Guo H. et al., 2015. Inflammasomes: mechanism of action, role in disease, and therapeutics. Nat Med. 21(7):677-87; Ren, Honglei, et al. “Selective NLRP3 (Pyrin Domain—Containing Protein 3) Inflammasome Inhibitor Reduces Brain Injury After Intracerebral Hemorrhage.” Stroke (2017): STROKEAHA-117).

In some embodiments, an inhibitor of the NLRP3 inflammasome is Bay11-7082, which has the structure as follows:

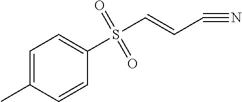

and was reported to selectively inhibit NLRP3 inflammasome activity in macrophages independent of their inhibitory effect on NF-κB activity (Juliana C. et al, 2010. Anti-inflammatory Compounds Parthenolide and Bay11-7082 Are Direct Inhibitors of the Inflammasome. J. Biol Chem. 285(13): 9792-9802].

In some embodiments, an inhibitor of the NLRP3 inflammasome is Glybenclamide (also known as glyburide), which has the structure as follows:

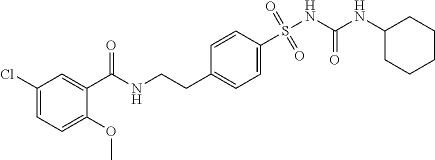 [SMILES: COc1ccc(Cl)cc1C(=O)NCCc1ccc(S(=O)(=O)NC(=O)NC2CCCCC2)cc1]

which blocks the maturation of caspase-1 and pro-IL-1β by inhibiting the K+ efflux (Laliberte R E. et al., 1999. ATP treatment of human monocytes promotes caspase-1 maturation and externalization. J Biol Chem. 274(52):36944-51). Glybenclamide also potently blocks the activation of the NRLP3 inflammasome induced by PAMPs, DAMPs and crystalline substances (Lamkanfi M. et al., 2009. Glyburide inhibits the Cryopyrin/Nalp3 inflammasome. J. Cell Biol., 187: 61-70; Dostert C. et al., 2009. Malarial hemozoin is a Nalp3 inflammasome activating danger signal. PLoS One. 4(8): e6510).

In some embodiments, an inhibitor of the NLRP3 inflammasome is isoliquiritigenin (also known as ILG), which has the structure as follows:

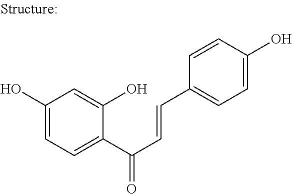 [SMILES: O=C(/C=C/c1ccc(O)cc1)c1ccc(O)cc1O]

which is a chalcone-type flavonoid isolated from licorice root (Glycyrrhiza uralensis) and was reported to inhibit NLRP3-activated ASC oligomerization (Honda H. et al., 2014. Isoliquiritigenin is a potent inhibitor of NLRP3 inflammasome activation and diet-induced adipose tissue inflammation. J Leukoc Biol. 96(6):1087-100). NLRP3-dependent IL-1β production has been inhibited with low concentrations of Isoliquiritigenin (1 to 10 μM), and demonstrates that Isoliquiritigenin can block the NLRP3 inflammasome at both the priming step and the activation step.

In some embodiments, an inhibitor of the NLRP3 inflammasome is 6673-34-0; (5-chloro-2-methoxy-N-[2-(4-sulfamoylphenyl)-ethyl]-benzamide)) which is disclosed in US application US20160052876, which is incorporated herein in its entirety by reference. In some embodiments, the inhibitor of the NLRP3 inflammasome is any of the small molecule compounds described in US20160052876, the contents of which are herein incorporated by reference in their entireties.

In some embodiments, an inhibitor of the NLRP3 inflammasome is cysteinyl leukotriene receptor antagonist, disclosed in Ozaki et al., 2015; Coll et al., 2011; Haerter et al., 2009 and U.S. Pat. No. 7,498,460, which are incorporated herein in its entirety by reference. The cysteinyl leukotriene receptor antagonist was reported to inhibit both NLRP3 and AIM2 inflammasome-induced IL-1 processing, by preventing ASC oligomerization and it also appears to have further roles in innate immune responses, different from its role of adaptor for inflammasome formation (Ozaki et al., 2015).

In some embodiments, small-molecule inhibitors targeting NLRP3 and AIM2 have been characterized and widely described in (Ozaki et al., 2015). The large majority of these are pharmacologic inhibitors that have been repurposed to target the inflammasome (Guo et al., 2015) and they include: Parthenolide (Juliana et al., 2010), Bay 11-708 (Juliana et al., 2010), CRID3 (Coll et al., 2011), Auranofin (Isakov et al., 2014), Isoliquiritigenin (Honda et al., 2014), 3,4-methylenedioxy-*-nitrostyrene (He et al., 2014), Cyclopentenone prostaglandin 15d-PJ2 (Maier et al., 2015) and 25-Hydroxycholesterol (25-HC) (Reboldi et al., 2014). Moreover, type I interferon has been shown to also suppress inflammasome activation with a poorly understood mechanism (Guarda et al., 2011). However, recently it has been demonstrated that an IFN-stimulated gene product, cholesterol 25-hydroxylase (Ch25h), antagonizes both Illb transcription and NLRP3, NLRC4 and AIM2 inflammasome activation, indicating that Ch25h has a broad inhibitory activity of multiple inflammasomes (Reboldi et al., 2014).

NLRP3 is encoded by NCBI accession numbers NM004895.1 (SEQ ID NO: 530), NM_183395 (SEQ ID NO: 531), NM_001079821 (SEQ ID NO: 532), NM_001127461 (SEQ ID NO: 533) and NM_001127462 (SEQ ID NO: 534). Here, the translation initiation codon in the NLRP3 is preferably the codon located 6 nucleotides downstream of the translation initiation codon described in each of these NCBI accession numbers. Examples of the mutant NLRP3 gene include NLRP3 gene wherein adenine at position 1709 counted from the translation initiation codon (in the case of the coding region shown in the NCBI accession numbers, position 1715 counted from the translation initiation codon) is guanine, cytosine at position 1043 (position 1049 in the coding region shown in the NCBI accession numbers) counted from the translation initiation codon is thymine, or guanine at position 587 (position 593 in the coding region shown in the NCBI accession numbers) counted from the translation initiation codon is adenine. The NLRP3 is preferably the one wherein the nucleotide at position 1079 is mutated to guanine. As one of skill in the art will appreciate, variants of the NLRP3 gene may exist which encode functionally equivalent NLRP3 which maintain function, at least in part, to activate caspase-1 and/or to promote the maturation of inflammatory cytokines such as Interleukin 1β and Interleukin 18. Such functionally equivalent NLRP3 may, thus, incorporate amino acid substitutions, deletions or additions that do not abolish activity.

In some embodiments of the compositions and methods described herein, an inhibitor of NLRP3 inflammasome is an RNA inhibitor (RNAi) of NLRP3, such as an siRNA specific for NLRP3. In some embodiments of the compositions and methods described herein, the RNA inhibitor of NLRP3 is encoded by the ceDNA. A NLRP3 siRNA can be commercially available, e.g., SI03060323 (Qiagen®).

In some embodiments, an inhibitor of NLRP3 is a RNAi encoded in a ceDNA. In avoidance of any doubt, the amino acid sequence of human NLRP3 protein corresponds to NM 004895.1 (SEQ ID NO: 539) and as is follows:

(SEQ ID NO: 539)
MKMASTRCKLARYLEDLEDVDLKKFKMHLEDYPPQKGCIPLPRG
QTEKADHVDLATLMIDFNGEEKAWAMAVWIFAAINRRDLYEKAKRDEPK
WGSDNARVSNPTVICQEDSIEEEWMGLLEYLSRISICKMKKDYRKKYRK
YVRSRFQCIEDRNARLGESVSLNKRYTRLRLIKEHRSQQEREQELLAIG
KTKICESPVSPIKMELLFDPDDEHSEPVHIVVFQGAAGIGKTILARKMM
LDWASGTLYQDRFDYLFYIHCREVSLVTQRSLGDLIMSCCPDPNPPIHK
IVRKPSRILFLMDGFDELQGAFDEHIGPLCIDWQKAERGDILLSSLIRK
KLLPEASLLITTRPVALEKLQHLLDHPRHVEILGFSEAKRKEYFFKYFS
DEAQARAAFSLIQENEVLFTMCFIPLVCWIVCIGLKQQMESGKSLAQTS
KITTAVYVFFLSSLLQPRGGSQEHGLCAHLWGLCSLAADGIWNQKILFE
ESDLRNHGLQKADVSAFLRMNLFQKEVDCEKFYSFIHMTFQEFFAAMYY
LLEEEKEGRINVPGSRLKLPSRDVIVLLENYGKFEKGYLIFVVRFLFGL
VNQERTSYLEKKLSCKISQQIRLELLKWIEVKAKAKKLQIQPSQLELFY
CLYEMQEEDFVQRAMDYFPKIEINLSTRMDHMVSSFCIENCHRVESLSL
GFLHNMPKEEEEEEKEGRHLDMVQCVLPSSSHAACSHGLVNSHLTSSFC
RGLFSVLSTSQSLTELDLSDNSLGDPGMRVLCETLQHPGCNIRRLWLGR
CGLSHECCFDISLVLSSNQKLVELDLSDNALGDFGIRLLCVGLKHLLCN
LKKLWLVSCCLISACCQDLASVLSTSHSLTRLYVGENALGDSGVAILCE
KAKNPQCNLQKLGLVNSGLTSVCCSALSSVLSTNQNLTHLYLRGNILGD
KGIKLLCEGLLHPDCKLQVLELDNCNLISHCCWDLSTLLTSSQSLRKLS
LGNNDLGDLGVMMECEVLKQQSCLLQNLGLSEMYENYETKSALETLQEE
KPELTVVFEPSW

The human NLRP3 protein is encoded by the NLRP3 gene comprising nucleic acid sequences NM_004895.1 (SEQ ID NO: 530), NM_183395 (SEQ ID NO: 531), NM_001079821 (SEQ ID NO: 532), NM_001127461 (SEQ ID NO: 533) and NM001127462 (SEQ ID NO: 534), and the human NLRP3 protein has an amino acid of NM004895 (SEQ ID NO: 539).

NLRP3 inhibitors further include antisense polynucleotides, which can be used to inhibit NLRP3 gene transcription and thereby NLRP3 inflammasome activation. Polynucleotides that are complementary to a segment of an NLRP3-encoding polynucleotide (e.g., a polynucleotide as set forth in SEQ ID NO: 530-534) are designed to bind to NLRP3-encoding mRNA and to inhibit translation of such mRNA. Antisense polynucleotides can be encoded by a ceDNA vector as disclosed herein, and can optionally, be operatively linked to a tissue specific or inducible promoter as disclosed herein.

Inhibition of the NLRP3 mRNA can be by gene silencing RNAi molecules according to methods commonly known by a skilled artisan. For example, a gene silencing siRNA oligonucleotide duplexes targeted specifically to human NLRP3 (NM_004895.1) can readily be used to knockdown NLRP3 expression. NLRP3 mRNA can be successfully targeted using siRNAs; and other siRNA molecules may be readily prepared by those of skill in the art based on the known sequence of the target mRNA. Accordingly, in avoidance of any doubt, one of ordinary skill in the art can design nucleic acid inhibitors, such as RNAi (RNA silencing) agents to the nucleic acid sequence of NM 004895.1 which is as follows:

| (SEQ ID NO: 530) | |
|---|---|
| 1 | gtagatgagg aaactgaagt tgaggaatag tgaagagttt gtccaatgtc atagccccgt |
| 61 | aatcaacggg acaaaaattt tcttgctgat gggtcaagat ggcatcgtga agtggttgtt |
| 121 | caccgtaaac tgtaatacaa tcctgtttat ggatttgttt gcatattttt ccctccatag |
| 181 | ggaaaccttt cttccatggc tcaggacaca ctcctggatc gagccaacag gagaactttc |
| 241 | tggtaagcat ttggctaact tttttttttt tgagatggag tcttgctgtg tcgcctaggc |
| 301 | tggagtgcag tggcgtgatc ttggctcact gcagcctcca cttcccgggt tcaatcaatt |
| 361 | ctcctacctc aacttcctga gtagctggga ttacaggcgc ccgccaccac acccggctca |
| 421 | tttttgtact tttagtagag acacagtttt gccatgttgg ccaggctggt cttgaattcc |
| 481 | tcagctcagg tgatctgcct gccttggcct ctcaaagtgc tgggattaca ggcgtgagcc |
| 541 | actgtgcccg gccttggcta acttttcaaa attaaagatt ttgacttgtt acagtcatgt |
| 601 | gacatttttt tctttctgtt tgctgagttt ttgataattt atatctctca aagtggagac |
| 661 | tttaaaaaag actcatccgt gtgccgtgtt cactgcctgg tatcttagtg tggaccgaag |
| 721 | cctaaggacc ctgaaaacag ctgcagatga agatggcaag cacccgctgc aagctggcca |
| 781 | ggtacctgga ggacctggag gatgtggact tgaagaaatt taagatgcac ttagaggact |
| 841 | atcctcccca gaagggctgc atccccctcc cgaggggtca gacagagaag gcagaccatg |
| 901 | tggatctagc cacgctaatg atcgacttca atggggagga gaaggcgtgg gccatggccg |
| 961 | tgtggatctt cgctgcgatc aacaggagag acctttatga gaaagcaaaa agagatgagc |
| 1021 | cgaagtgggg ttcagataat gcacgtgttt cgaatcccac tgtgatatgc caggaagaca |
| 1081 | gcattgaaga ggagtggatg ggtttactgg agtacctttc gagaatctct atttgtaaaa |
| 1141 | tgaagaaaga ttaccgtaag aagtacagaa agtacgtgag aagcagattc cagtgcattg |
| 1201 | aagacaggaa tgcccgtctg ggtgagagtg tgagcctcaa caaacgctac acacgactgc |
| 1261 | gtctcatcaa ggagcaccgg agccagcagg agagggagca ggagcttctg gccatcggca |
| 1321 | agaccaagac gtgtgagagc cccgtgagtc ccattaagat ggagttgctg tttgaccccg |
| 1381 | atgatgagca ttctgagcct gtgcacaccg tggtgttcca gggggcggca gggattggga |
| 1441 | aaacaatcct ggccaggaag atgatgttgg actgggcgtc ggggacactc taccaagaca |
| 1501 | ggtttgacta tctgttctat atccactgtc gggaggtgag ccttgtgaca cagaggagcc |
| 1561 | tgggggacct gatcatgagc tgctgccccg acccaaaccc acccatccac aagatcgtga |
| 1621 | gaaaaccctc cagaatcctc ttcctcatgg acggcttcga tgagctgcaa ggtgcctttg |
| 1681 | acgagcacat aggaccgctc tgcactgact ggcagaaggc cgagcgggga gacattctcc |
| 1741 | tgagcagcct catcagaaag aagctgcttc ccgaggcctc tctgctcatc accacgagac |
| 1801 | ctgtggccct ggagaaactg cagcacttgc tggaccatcc tcggcatgtg gagatcctgg |
| 1861 | gtttctccga ggccaaaagg aaagagtact tcttcaagta cttctctgat gaggcccaag |
| 1921 | ccagggcagc cttcagtctg attcaggaga acgaggtcct cttcaccatg tgcttcatcc |
| 1981 | ccctggtctg ctggatcgtg tgcactggac tgaaacagca gatggagagt ggcaagagcc |
| 2041 | ttgcccagac atccaagacc accaccgcgg tgtacgtctt cttcctttcc agtttgctgc |
| 2101 | agccccgggg agggagccag gagcacggcc tctgcgccca cctctggggg ctctgctctt |
| 2161 | tggctgcaga tggaatctgg aaccagaaaa tcctgtttga ggagtccgac ctcaggaatc |
| 2221 | atggactgca gaaggcggat gtgtctgctt tcctgaggat gaacctgttc caaaaggaag |
| 2281 | tggactgcga gaagttctac agcttcatcc acatgacttt ccaggagttc tttgccgcca |
| 2341 | tgtactacct gctggaagag gaaaaggaag gaaggacgaa cgttccaggg agtcgtttga |
| 2401 | agcttcccag ccgagacgtg acagtccttc tggaaaacta tggcaaattc gaaaaggggt |
| 2461 | atttgatttt tgttgtacgt ttcctctttg gcctggtaaa ccaggagagg acctcctact |
| 2521 | tggagaagaa attaagttgc aagatctctc agcaaatcag gctggagctg ctgaaatgga |
| 2581 | ttgaagtgaa agccaaagct aaaaagctgc agatccagcc cagccagctg gaattgttct |
| 2641 | actgtttgta cgagatgcag gaggaggact tcgtgcaaag ggccatggac tatttcccca |
| 2701 | agattgagat caatctctcc accagaatgg accacatggt ttcttccttt tgcattgaga |
| 2761 | actgtcatcg ggtggagtca ctgtccctgg ggtttctcca taacatgccc aaggaggaag |
| 2821 | aggaggagga aaaggaaggc cgacaccttg atatggtgca gtgtgtcctc ccaagctcct |
| 2881 | ctcatgctgc ctgttctcat ggattggtga acagccacct cacttccagt ttttgccggg |
| 2941 | gcctcttttc agttctgagc accagccaga gtctaactga attggacctc agtgacaatt |
| 3001 | ctctggggga cccagggatg agagtgttgt gtgaaacgct ccagcatcct ggctgtaaca |
| 3061 | ttcggagatt gtggttgggg cgctgtggcc tctcgcatga gtgctgcttc gacatctcct |
| 3121 | tggtcctcag cagcaaccag aagctggtgg agctggacct gagtgacaac gccctcggtg |
| 3181 | acttcggaat cagacttctg tgtgtgggac tgaagcacct gttgtgcaat ctgaagaagc |
| 3241 | tctggttggt cagctgctgc ctcacatcag catgttgtca ggatcttgca tcagtattga |
| 3301 | gcaccagcca ttccctgacc agactctatg tgggggagaa tgccttggga gactcaggag |
| 3361 | tcgcaatttt atgtgaaaaa gccaagaatc cacagtgtaa cctgcagaaa ctggggttgg |
| 3421 | tgaattctgg ccttacgtca gtctgttgtt cagctttgtc ctcggtactc agcactaatc |
| 3481 | agaatctcac gcacctttac ctgcgaggca acactctcgg agacaagggg atcaaactac |
| 3541 | tctgtgaggg actcttgcac cccgactgca agcttcaggt gttggaatta gacaactgca |
| 3601 | acctcacgtc acactgctgc tgggatcttt ccacacttct gacctccagc cagagcctgc |
| 3661 | gaaagctgag cctgggcaac aatgacctgg gcgacctggg ggtcatgatg ttctgtgaag |
| 3721 | tgctgaaaca gcagagctgc ctcctgcaga acctggggtt gtctgaaatg tatttcaatt |
| 3781 | atgagacaaa aagtgcgtta gaaacacttc aagaagaaaa gcctgagctg accgtcgtct |
| 3841 | ttgagccttc ttggtaggag tggaaacggg gctgccagac gccagtgttc tccggtccct |
| 3901 | ccagctgggg gccctcaggt ggagagagct gcgatccatc caggccaaga ccacagctct |
| 3961 | gtgatccttc cggtggagtg tcggagaaga gagcttgccg acgatgcctt cctgtgcaga |
| 4021 | gcttgggcat ctcctttacg ccagggtgag gaagacacca ggacaatgac agcatcgggt |
| 4081 | gttgttgtca tcacagcgcc tcagttagag gatgttcctc ttggtgacct catgtaatta |
| 4141 | gctcattcaa taaagcactt tctttatttt tctcttctct gtctaacttt ctttttccta |
| 4201 | tcttttttct tctttgttct gtttactttt gctcatatca tcattcccgc tatctttcta |
| 4261 | ttaactgacc ataacacaga actagttgac tatatattat gttgaaattt tatggcagct |
| 4321 | atttatttat ttaaattttt tgtaacagtt ttgttttcta ataagaaaaa tccatgcttt |
| 4381 | ttgtagctgg ttgaaaattc aggaatatgt aaaacttttt ggtatttaat taaattgatt |
| 4441 | ccttttctta attttaaaaa aaaaaaaaaa |

In some embodiments, a NLRP3 inflammasome inhibitor is a siRNA, thereby inhibiting the mRNA of the NLRP3 inflammasome. In some embodiments, a NLRP3 inflammasome inhibitor is GUGCAUUGAAGACAGGAAUTT (SEQ ID NO: 540) (Wang et al., Laboratory Invest. (2017) 97: 922-934, which is incorporated herein in its entirety by reference) which inhibits human NLRP3 expression or a fragment or a homologue thereof of at least 50%, or at least 60% or at least 70% or at least 80% or at least 90% identical thereto. In some embodiments, a NLRP3 inflammasome inhibitor is a commercially available siRNA, such as available from Santa Cruz® (cat # sc-40327).

In some embodiments, a NLRP3 inflammasome inhibitor is a RNAi that is complementary to a RNAi target sequence in the Human NM_001079821.2, NCBI gene 114548 (NLRP3). A RNAi agent that inhibits NLRP3 can be a nucleic acid that is complementary to between 17-21 consecutive bases of SEQ ID NO: 541-551, shown Table 5A.

TABLE 5A
Target sequences for RNAi for inhibition
of NLRP3:

| | SEQ ID | |
|---|---|---|
| Target sequence | NO: | Clone ID |
| GGCTGTAACATTCGGAGATTG | 541 | TRCN0000419896 |
| TCATCATTCCCGCTATCTTTC | 542 | TRCN0000420883 |
| CCGTAAGAAGTACAGAAAGTA | 543 | TRCN0000062723 |
| GAGACTCAGGAGTCGCAATTT | 544 | TRCN0000431574 |
| CCTCATGTAATTAGCTCATTC | 545 | TRCN0000427726 |
| GTGGATCTAGCCACGCTAATG | 546 | TRCN0000432208 |
| CCACAGTGTAACCTGCAGAAA | 547 | TRCN0000062725 |
| CCAGCCAGAGTCTAACTGAAT | 548 | TRCN0000062724 |
| GCGTTAGAAACACTTCAAGAA | 549 | TRCN0000062726 |
| GCTGGAATTGTTCTACTGTTT | 550 | TRCN0000062727 |
| CCACATGACTTTCCAGGAGTT | 551 | TRCN0000101069 |

In some embodiments, a NLRP3 inflammasome inhibitor is a siRNA agent, Exemplary siRNA sequences which inhibit NLRP3 are shown in Table 5B.

TABLE 5B
Exemplary siRNA which inhibit NLRP3

| Clone ID | Target Seq | Forward and Reverse Oligo Sequences |
|---|---|---|
| TRCN0000419896 | GGCTGTAACATTCG | Forward: |
| | GAGATTG (SEQ ID | CCGGGGCTGTAACATTCGGAGATTGCTCGAGCAATCTCCG |
| | NO: 552) | AATGTTACAGCCTTTTTG (SEQ ID NO: 553) |
| | | Reverse: |
| | | AATTCAAAAAGGCTGTAACATTCGGAGATTGCTCGAGCAA |
| | | TCTCCGAATGTTACAGCC (SEQ ID NO: 554) |
| TRCN0000420883 | TCATCATTCCCGCTA | Forward: CCGGTCATCATTCCCGCTATCTTTCCTCGAGGAA |
| | TCTTTC (SEQ ID NO: | AGATAGCGGGAATGATGATTTTTG (SEQ ID NO: 556) |
| | 555) | Reverse: |
| | | AATTCAAAAATCATCATTCCCGCTATCTTTCCTCGAGGAAA |
| | | GATAGCGGGAATGATGA (SEQ ID NO: 557) |
| TRCN0000062723 | CCGTAAGAAGTACA | Forward: |
| | GAAAGTA (SEQ ID | CCGGCCGTAAGAAGTACAGAAAGTACTCGAGTACTTTCTG |
| | NO: 558) | TACTTCTTACGGTTTTTG (SEQ ID NO: 559) |
| | | Reverse: |
| | | AATTCAAAAACCGTAAGAAGTACAGAAAGTACTCGAGTAC |
| | | TTTCTGTACTTCTTACGG (SEQ ID NO: 560) |
| TRCN0000431574 | GAGACTCAGGAGTC | Forward: |
| | GCAATTT (SEQ ID | CCGGGAGACTCAGGAGTCGCAATTTCTCGAGAAATTGCGA |
| | NO: 561) | CTCCTGAGTCTCTTTTTG (SEQ ID NO: 562) |
| | | Reverse: |
| | | AATTCAAAAAGAGACTCAGGAGTCGCAATTTCTCGAGAAA |
| | | TTGCGACTCCTGAGTCTC (SEQ ID NO: 563) |
| TRCN0000427726 | CCTCATGTAATTAGC | Forward: |
| | TCATTC (SEQ ID NO: | CCGGCCTCATGTAATTAGCTCATTCCTCGAGGAATGAGCTA |
| | 564) | ATTACATGAGGTTTTTG (SEQ ID NO: 565) |
| | | Reverse: |
| | | AATTCAAAAACCTCATGTAATTAGCTCATTCCTCGAGGAAT |
| | | GAGCTAATTACATGAGG (SEQ ID NO: 566) |
| TRCN0000432208 | GTGGATCTAGCCAC | Forward: |
| | GCTAATG (SEQ ID | CCGGGTGGATCTAGCCACGCTAATGCTCGAGCATTAGCGT |
| | NO: 567) | GGCTAGATCCACTTTTTG (SEQ ID NO: 568) |
| | | Reverse: |
| | | AATTCAAAAAGTGGATCTAGCCACGCTAATGCTCGAGCAT |
| | | TAGCGTGGCTAGATCCAC (SEQ ID NO: 569) |
| TRCN0000062725 | CCACAGTGTAACCTG | Forward: |
| | CAGAAA (SEQ ID | CCGGCCACAGTGTAACCTGCAGAAACTCGAGTTTCTGCAG |
| | NO: 570) | GTTACACTGTGGTTTTTG (SEQ ID NO: 570) |
| | | Reverse: |
| | | AATTCAAAAACCACAGTGTAACCTGCAGAAACTCGAGTTT |
| | | CTGCAGGTTACACTGTGG (SEQ ID NO: 571) |
| TRCN0000062724 | CCAGCCAGAGTCTAA | Forward: |
| | CTGAAT (SEQ ID NO: | CCGGCCAGCCAGAGTCTAACTGAATCTCGAGATTCAGTTA |
| | 572) | GACTCTGGCTGGTTTTTG (SEQ ID NO: 573) |
| | | Reverse: |
| | | AATTCAAAAACCAGCCAGAGTCTAACTGAATCTCGAGATT |
| | | CAGTTAGACTCTGGCTGG (SEQ ID NO: 574) |
| TRCN0000062726 | GCGTTAGAAACACTT | Forward: |
| | CAAGAA (SEQ ID | CCGGGCGTTAGAAACACTTCAAGAACTCGAGTTCTTGAAG |
| | NO: 575) | TGTTTCTAACGCTTTTTG (SEQ ID NO: 576) |
| | | Reverse: |
| | | AATTCAAAAAGCGTTAGAAACACTTCAAGAACTCGAGTTC |
| | | TTGAAGTGTTTCTAACGC (SEQ ID NO: 577) |
| TRCN0000062727 | GCTGGAATTGTTCTA | Forward: |
| | CTGTTT (SEQ ID NO: | CCGGGCTGGAATTGTTCTACTGTTTCTCGAGAAACAGTAG |
| | 578) | AACAATTCCAGCTTTTTG (SEQ ID NO: 579) |
| | | Reverse: |
| | | AATTCAAAAAGCTGGAATTGTTCTACTGTTTCTCGAGAAAC |
| | | AGTAGAACAATTCCAGC (SEQ ID NO: 580) |
| TRCN0000101069 | CCACATGACTTTCCA | Forward: |
| | GGAGTT (SEQ ID | CCGGCCACATGACTTTCCAGGAGTTCTCGAGAACTCCTGG |
| | NO: 581) | AAAGTCATGTGGTTTTTG (SEQ ID NO: 582) |
| | | Reverse: |
| | | AATTCAAAAACCACATGACTTTCCAGGAGTTCTCGAGAACT |
| | | CCTGGAAAGTCATGTGG (SEQ ID NO: 583) |
| TRCN0000191875 | GAAAGCCAAAGCTA | Forward: |
| | AGAAGTA (SEQ ID | CCGGGAAAGCCAAAGCTAAGAAGTACTCGAGTACTTCTTA |
| | NO: 584) | GCTTTGGCTTTCTTTTTG (SEQ ID NO: 585) |
| | | Reverse: |
| | | AATTCAAAAAGAAAGCCAAAGCTAAGAAGTACTCGAGTAC |
| | | TTCTTAGCTTTGGCTTTC (SEQ ID NO: 586) |

In some embodiments, a NLRP3 inflammasome inhibitor is a miRNA (miR) that inhibits the expression of NLRP3, or an agonist of a miR that inhibits NLRP3 expression. Exemplary miRs that inhibit NLRP3 are miR-9 and miR-223.

miR-9 inhibits NLRP3 inflammaosome activation (Wang, Yue, et al. “MicroRNA-9 inhibits NLRP3 inflammasome activation in human atherosclerosis inflammation cell models through the JAK1/STAT signaling pathway.” Cellular Physiology and Biochemistry 41.4 (2017): 1555-1571). Accordingly, pre-miR-9 (MiR-9 precursor) or miR-9 can be used to inhibit NLRP3. The sequence of mature miR-9 (MIMAT0000441) is 5′-UCU UUG GUU AUC U AG CUG UAU GA-3′ (SEQ ID NO: 587). hsa-miR-9-5p (UCUUUGGUUAUCUAGCUGUAUGA) (SEQ ID NO: 588). In some embodiments, a NLRP3 inflammasome inhibitor is the miR-9 agonist SQ22538 (SQ; 9-(tetrahydro-2-furanyl)-9H-purin-6-amine), which was reported to increase the expression of miR-9 (Ham, Onju, et al. “Small molecule-mediated induction of miR-9 suppressed vascular smooth muscle cell proliferation and neointima formation after balloon injury.” Oncotarget 8.55 (2017): 93360). The formula of SQ22538 is as follows:

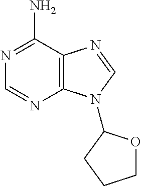 [SMILES: Nc1ncnc2c1ncn2C1CCCO1]

miR-223 inhibits the activity of the NLRP3 inflammasome. (Bauernfeind, Franz, et al. “NLRP3 inflammasome activity is negatively controlled by miR-223.” The Journal of Immunology 189.8 (2012): 4175-4181; Feng, Zunyong, et al. “Ly6G+ neutrophil-derived miR-223 inhibits the NLRP3 inflammasome in mitochondrial DAMP-induced acute lung injury.” Cell death & disease 8.11 (2017): e3170). miR-223 can be synthesized as mmu-miR-223. At least one, or 2- or 3 or 4 blocks of a sequence complementary to

miR-223
(5′-TGGGGTATTTGACAAACTGACA-3′ (SEQ ID NO: 589)
can be used to inhibit NLRP3.
cbn-mir-233 MI0024890 has the sequence of:
(SEQ ID NO: 590)
UCGCCCAUCCCGUUGUUCCAAUAUUCCAACAACAAGUGAUUAUUGAGCA
AUGCGCAUGUGCGG;
cbr-mir-233 MI0000530 has the sequence of:
(SEQ ID NO: 591)
AAGCAUUUUUCUGUCCCGCGCAUCCCUUUGUUCCAAUAUUCAAACCAGU
AGAAAGAUUAUUGAGCAAUGCGCAUGUGCGGGACAGAUUGAAUAGCUG;
cel-mir-233 MI0000308 has the sequence of:
(SEQ ID NO: 592)
AUAUAGCAUCUUUCUGUCUCGCCCAUCCCGUUGCUCCAAUAUUCUAACA
ACAAGUGAUUAUUGAGCAAUGCGCAUGUGCGGGAUAGACUGAUGGCUGC;
crm-mir-233 MI0011059 has the sequence of:
(SEQ ID NO: 593)
UGAAGCGUCUCUCUGUCCCGCUCAUCCUGUUGUUCCAAUAUUCCAACAG
CCCAGUGAUUAUUGAGCAAUGCGCAUGUGCGGGACAGAUUGUAUGCUGC
CAU.

In some embodiments, a NLRP3 inflammasome inhibitor is an anti-miRNA (anti-miR) that inhibits the expression of a miR that suppresses NLRP3 expression or function. Exemplary anti-miRs are anti-miR-22 and anti-miR-33. miR22 has been demonstrated to sustain expression of NLRP3 (Li, S., et al., “MiR-22 sustains NLRP3 expression and attenuates H. pylori-induced gastric carcinogenesis.” Oncogene 37.7 (2018): 884). The mature sequence of miR-22 is hsa-miR-22 (hsa-miR-22-5p MIMAT000449) is: AGUUCUUCAGUGGCAAGCUUUA (SEQ ID NO: 594), with the stem loop sequence as follows:

hsa-mir-22 MI0000078 has the sequence of:
(SEQ ID NO: 595)
GGCUGAGCCGCAGUAGUUCUUCAGUGGCAAGCUUUAUGUCCUGACCCAG
CUAAAGCUGCCAGUUGAAGAACUGUUGCCCUCUGCC.

miR-33 has been reported to upregulate the expression of NLRP3 mRNA and protein as well as caspase-1 activity in primary macrophages (Xie, Qingyun, et al. “MicroRNA-33 regulates the NLRP3 inflammasome signaling pathway in macrophages.” Molecular medicine reports 17.2 (2018): 3318-3327). The mature sequence of miR-33 is mmu-miR-33-5p or MIMAT0000667; and is: GUGCAUUGUAGUUGCAUUGCA (SEQ ID NO: 596); with the stem loop sequence as follows:

mmu-mir-33 MI0000707:
(SEQ ID NO: 597)
CUGUGGUGCAUUGUAGUUGCAUUGCAUGUUCUGGCAAUACCUGUGCAAU
GUUUCCACAGUGCAUCACGG

Accordingly, in some embodiments, an inhibitor of NLRP3 is an anti-miR-22 that is complementary to at least a portion e.g., 15-25 mers of SEQ ID NO: 594 or SEQ ID NO: 595, or an anti-miR-33 that is complementary to at least a portion e.g., 15-21 mers of SEQ ID NO: 596 or SEQ ID NO: 597.

In some embodiments of the compositions and methods described herein, an inhibitor of NLRP3 inflammasome is an anti-human NLRP3 (catalog no. AF6789) from R&D Systems (Minneapolis, Minn.). In some embodiments, the antibody inhibitor of NLRP3 is encoded by the ceDNA.

In some embodiments of the compositions and methods described herein, an inhibitor of NLRP3 is an antibody or antigen-binding fragment that binds NLRP3. In some embodiments of the compositions and methods described herein, the antibody or antigen-binding fragment that binds NLRP3 is encoded by the ceDNA.

A NLRP3 inflammasome inhibitor refers to compounds which inhibit or at least reduce the activity of the inflammasome, including glyburide and functionally equivalent precursors or derivatives thereof, caspase-1 inhibitors, adenosine monophosphate-activated protein kinase (AMPK) activators and P2X7 inhibitors. Inhibition of NLRP3 inflammasome may be achieved by a single compound or a combination of compounds that inhibit the inflammasome or caspase-1, but which do not result in changes to cytochrome P450 (cyp) enzyme activity, including cyp isoforms, 3A4, 2C9 and 2C19, that would adversely affect the metabolism of statins and thereby reduce the bioavailability of statins.

### Inhibitors of the AIM2 Inflammasome Pathway

In some embodiments, an inflammasome antagonist inhibits AIM2. AIM2, alternatively known as PISA, is a 343 amino acid polypeptide (see Genbank accession number AF024714.1; RefSeq accession number NP_004824.1) (SEQ ID NO: 598). AIM2 is a member of the IFI20X/IF116 family, and is known to expressed in the spleen, the small intestine, peripheral blood leukocytes, and the testis. AIM2 contains a PYD domain, which is involved in interaction with ASC, as well as a HIN200 domain that is involved in interaction with dsDNA. AIM2 plays a putative role in tumorigenic reversion and may control cell proliferation. Expression of AIM2 is induced by interferon-gamma.

In some embodiments of the compositions and methods described herein, an inhibitor of AIM2 is an antibody or antigen-binding fragment that binds AIM2. In some embodiments of the compositions and methods described herein, the antibody or antigen-binding fragment that binds NLRP3 is encoded by the ceDNA. Inhibitors of AIM2 are disclosed in Farshchian et al., Oncotarget 2017; 8(28); 45825-45836, which is incorporated herein in its entirety by reference.

In some embodiments, the inhibitor of the AIM2 inflammasome an anti-human ASC monoclonal antibody (clone 23-4, MBL, Nagoya, Japan) which has been reported to interfere with PYD of ASC. In some embodiments, the inhibitor of the AIM2 inflammasome an anti-human AIM2 (catalog no. 8055) antibody (Cell Signaling Technology® (Beverly, Mass.). In some embodiments, the inhibitor of the AIM2 inflammasome is an endogenous AIM2 inhibitor, such as the pyrin-containing proteins, recently described by (Khare et al., 2014; de Almeida et al., 2015), or antimicrobial cathelicidin peptides, reported by Schauber and colleagues (Dombrowski et al., 2011). In some embodiments, the inhibitor of the AIM2 inflammasome is any compound disclosed in the minireview by Miriam Canavase “the duality of AIM2 inflammasome: A focus on its role in autoimmunity and Skin diseases. Am. J. Pharm & Toxicology; 2016).

In some embodiments, the inhibitor of the AIM2 inflammasome is P202, which is a p202 tetramer and reported to reduce AIM2 activation, and prevented dsDNA-dependent clustering of ASC and AIM2 inflammasome activation (Fernandes-Alnemri, Teresa, et al. “The AIM2 inflammasome is critical for innate immunity to Francisella tularensis.” Nature immunology 11.5 (2010): 385; Yin, Qian, et al. “Molecular mechanism for p202-mediated specific inhibition of AIM2 inflammasome activation.” Cell reports 4.2 (2013): 327-339). In some embodiments of the compositions and methods described herein, P202 is encoded by the ceDNA.

In some embodiments, the inhibitor of the AIM2 inflammasome is any of the small molecule compounds described in WO2017138586A, or US2013/0158100A1, the contents of each are herein incorporated by reference in their entireties.

In some embodiments of the compositions and methods described herein, an inhibitor of AIM2 is an RNA inhibitor of AIM2, such as an siRNA specific for AIM2. In some embodiments of the compositions and methods described herein, the RNA inhibitor of AIM2 is encoded by the ceDNA The human AIM2 protein is encoded by the AIM2 gene comprising nucleic acid sequence NM_004833.2 (SEQ ID NO: 600), and the human AIM2 protein has an amino acid of NP_004824.1 (SEQ ID NO: 598). AIM2 inhibitors further include antisense polynucleotides, which can be used to inhibit AIM2gene transcription and thereby AIM2 inflammasome activation. Polynucleotides that are complementary to a segment of an AIM2-encoding polynucleotide (e.g., a polynucleotide as set forth in SEQ ID NO: 600) are designed to bind to AIM2-encoding mRNA and to inhibit translation of such mRNA. Antisense polynucleotides can be encoded by a ceDNA vector as disclosed herein, and can optionally, be operatively linked to a tissue specific or inducible promoter as disclosed herein. Inhibition of the AIM2 mRNA can be by gene silencing RNAi molecules according to methods commonly known by a skilled artisan. For example, a gene silencing siRNA oligonucleotide duplexes targeted specifically to human AIM2 (NM_004833.2) can readily be used to knockdown AIM2 expression. AIM2 mRNA can be successfully targeted using siRNAs; and other siRNA molecules may be readily prepared by those of skill in the art based on the known sequence of the target mRNA. Accordingly, in avoidance of any doubt, one of ordinary skill in the art can design nucleic acid inhibitors, such as RNAi (RNA silencing) agents to the nucleic acid sequence of NM_004833.2 which is as follows:

| (SEQ ID NO: 600) | |
|---|---|
| 1 | atagacattt tcttctgtgg ctgctagtga gaacccaaac cagctcagcc aattagagct |
| 61 | ccagttgtca ctcctaccca cactgggcct gggggtgaag ggaagtgttt attaggggta |
| 121 | catgtgaagc cgtccagaag tgtcagagtc tttgtagctt tgaaagtcac ctaggttatt |
| 181 | tgggcatgct ctcctgagtc ctctgctagt taagctctct gaaaagaagg tggcagaccc |
| 241 | ggtttgctga tcgccccagg gatcaggagg ctgatcccaa agttgtcaga tggagagtaa |
| 301 | atacaaggag atactcttgc taacaggcct ggataacatc actgatgagg aactggatag |
| 361 | gtttaagttc tttctttcag acgagtttaa tattgccaca ggcaaactac atactgcaaa |
| 421 | cagaatacaa gtagctacct tgatgattca aaatgctggg gcggtgtctg cagtgatgaa |
| 481 | gaccattcgt atttttcaga agttgaatta tatgcttttg gcaaaacgtc ttcaggagga |
| 541 | gaaggagaaa gttgataagc aatacaaatc ggtaacaaaa ccaaagccac taagtcaagc |
| 601 | tgaaatgagt cctgctgcat ctgcagccat cagaaatgat gtcgcaaagc aacgtgctgc |
| 661 | accaaaagtc tctcctcatg ttaagcctga acagaaacag atggtggccc agcaggaatc |
| 721 | tatcagagaa gggtttcaga agcgctgttt gccagttatg gtactgaaag caaagaagcc |
| 781 | cttcacgttt gagacccaag aaggcaagca ggagatgttt catgctacag tggctacaga |
| 841 | aaaggaattc ttctttgtaa aagtttttaa tacactgctg aaagataaat tcattccaaa |
| 901 | gagaataatt ataatagcaa gatattatcg gcacagtggt ttcttagagg taaatagcgc |
| 961 | ctcacgtgtg ttagatgctg aatctgacca aaaggttaat gtcccgctga acattatcag |
| 1021 | aaaagctggt gaaaccccga agatcaacac gcttcaaact cagccccttg gaacaattgt |
| 1081 | gaatggtttg tttgtagtcc agaaggtaac agaaaagaag aaaaacatat tatttgacct |
| 1141 | aagtgacaac actgggaaaa tggaagtact gggggttaga aacgaggaca caatgaaatg |
| 1201 | taaggaagga gataaggttc gacttacatt cttcacactg tcaaaaaatg gagaaaaact |
| 1261 | acagctgaca tctggagttc atagcaccat aaaggttatt aaggccaaaa aaaaaacata |
| 1321 | gagaagtaaa aaggaccaat tcaagccaac tggtctaagc agcatttaat tgaagaatat |
| 1381 | gtgatacagc ctcttcaatc agattgtaag ttacctgaaa gctgcagttc acaggctcct |
| 1441 | ctctccacca aattaggata gaataattgc tggataaaca aattcagaat atcaacagat |
| | gatcacaata aacatctgtt tctcattcaa aaaaaaaaaa aaaaaaaaaa aaaaaaaa |

In some embodiments, an AIM2 inflammasome inhibitor is a siRNA, thereby inhibiting the mRNA of the AIM2 inflammasome. In some embodiments, an AIM2 inflammasome inhibitor is 5′-CCCGAAGATCAACACGCTTCA-3′ (SEQ ID NO: 601) or 5′-AAAGGTTAATGTCCCGCTGAA-3′ (SEQ ID NO: 665) (both from Farshchian et al. Oncotarget (2017) 8: 45825-45836) which inhibits human AIM2 expression or a fragment or a homologue thereof of at least 50%, or at least 60% or at least 70% or at least 80% or at least 90% identical thereto.

In some embodiments of the compositions and methods described herein, an inhibitor of AIM2 inflammasome is an RNA inhibitor of AIM2, such as an siRNA specific for AIM2. In some embodiments of the compositions and methods described herein, the RNA inhibitor of AIM2 is encoded by the ceDNA. An AIM2 siRNA can be commercially available, e.g., SI04261432 (Qiagen®); or RCN0000096104 (#1), TRCN0000096105 (#2), TRCN0000096106 (#3) from OpenBiosystems® (Huntsville, Ala.).

In some embodiments, the inhibitor of the AIM2 inflammasome is A151 (5′-TTAGGGTTAGGGTTAGGGTTAGGG-3′ (SEQ ID NO: 602) or C151 (5′-TTCAAATTCAAATTCAAATTCAAA-3′ (SEQ ID NO: 603) that is synthesized with a phosphorothioate (PO) backbone. A151 (also referred to as ODN TTAGGG) is a synthetic oligonucleotide (ODN) containing 4 repeats of the immunosuppressive TTAGGG (SEQ ID NO: 604) motif commonly found in mammalian telomeric DNA (Steinhagen F. et al., 2017. Suppressive oligodeoxynucleotides containing TTAGGG motifs inhibit cGAS activation in human monocytes. Eur J Immunol). A151 blocks AIM2 inflammasome activation in response to cytosolic dsDNA, but requires a phosphothioate (PO) backbone (Kaminsji et al., J Immunol 2013; 191:3876-3883, Synthetic Oligodeoxynucleotides Containing Suppressive TTAGGG Motifs Inhibit AIM2 Inflammasome Activation; Eichholz K. et al., 2016 Immune-Complexed Adenovirus Induce AIM2-Mediated Pyroptosis in Human Dendritic Cells. PLoS Pathog. 12(9): e1005871). In some embodiments, an inhibitor of the AIM2 inflammasome is A151 (5′-TTAGGGTTAGGGTTAGGGTTAGGG-3′ (SEQ ID NO: 602) or at least one repeat of TTAGGG (SEQ ID NO: 604), each with a phosphothioate (PO) backbone. In some embodiments, an inhibitor of the AIM2 inflammasome is A151 (5′-TTAGGGTTAGGGTTAGGGTTAGGG-3′ (SEQ ID NO: 602) or at least one repeat of TTAGGG (SEQ ID NO: 604), that does not have a phosphodiester (PE) backbone. In some embodiments of the compositions and methods described herein, an inhibitor of the AIM2 inflammasome is encoded by a ceDNA being administered to a subject (including, e.g. subsequent delivery of ceDNA). In some embodiments of the compositions and methods described herein, an inhibitor of the AIM2 inflammasome encoded by a ceDNA being administered to a subject is A151 (SEQ ID NO: 602).

In some embodiments, an AIM2 inflammasome inhibitor is a RNAi that is complementary to a RNAi target sequence in the Human NM_001348247.1 (SEQ ID NO: 566), NCBI gene 9447 (AIM2). A RNAi agent that inhibits AIM2 can be a nucleic acid that is complementary to between 17-21 consecutive bases of SEQ ID NO: 605-610, shown Table 5C.

TABLE 5C
Target sequences for RNAi for inhibition of AIM2:

| Target Seq | SEQ ID NO: | Clone ID |
|---|---|---|
| AGCCACTAAGTCAAGCTGAAA | 605 | TRCN0000107503 |
| CCAACTGGTCTAAGCAGCATT | 606 | TRCN0000107500 |
| GAAACGAGGACACAATGAAAT | 607 | TRCN0000413154 |
| GCCACTAAGTCAAGCTGAAAT | 608 | TRCN0000107502 |
| CTGGAGTTCATAGCACCATAA | 609 | TRCN0000107504 |
| CCCGCTGAACATTATCAGAAA | 610 | TRCN0000107501 |

In some embodiments, an AIM2 inflammasome inhibitor is a siRNA agent, Exemplary siRNA sequences which inhibit AIM2 are shown in Table 5D.

TABLE 5D
Exemplary siRNA which inhibit AIM2

| Clone ID | Target Seq | Forward and reverse Oligo Sequence |
|---|---|---|
| TRCN0000107503 | AGCCACTAAGTCAAGCT | Forward: |
| | GAAA (SEQ ID NO: 666) | CCGGAGCCACTAAGTCAAGCTGAAACTCGAGTTTCAGCTTGAC |
| | | TTAGTGGCTTTTTTG (SEQ ID NO: 667) |
| | | Reverse: |
| | | AATTCAAAAAAGCCACTAAGTCAAGCTGAAACTCGAGTTTCAG |
| | | CTTGACTTAGTGGCT (SEQ ID NO: 668) |
| TRCN0000107500 | CCAACTGGTCTAAGCAG | Forward: |
| | CATT (SEQ ID NO: 669) | CCGGCCAACTGGTCTAAGCAGCATTCTCGAGAATGCTGCTTAG |
| | | ACCAGTTGGTTTTTG (SEQ ID NO: 670) |
| | | Reverse: |
| | | AATTCAAAAACCAACTGGTCTAAGCAGCATTCTCGAGAATGCT |
| | | GCTTAGACCAGTTGG (SEQ ID NO: 671) |
| TRCN0000413154 | GAAACGAGGACACAATG | Forward: |
| | AAAT (SEQ ID NO: 672) | CCGGGAAACGAGGACACAATGAAATCTCGAGATTTCATTGTGT |
| | | CCTCGTTTCTTTTTG (SEQ ID NO: 673) |
| | | Reverse: |
| | | AATTCAAAAAGAAACGAGGACACAATGAAATCTCGAGATTTCA |
| | | TTGTGTCCTCGTTTC (SEQ ID NO: 674) |
| TRCN0000107502 | GCCACTAAGTCAAGCTG | Forward: |
| | AAAT (SEQ ID NO: 675) | CCGGGCCACTAAGTCAAGCTGAAATCTCGAGATTTCAGCTTGA |
| | | CTTAGTGGCTTTTTG (SEQ ID NO: 676) |
| | | Reverse: |
| | | AATTCAAAAAGCCACTAAGTCAAGCTGAAATCTCGAGATTTCA |
| | | GCTTGACTTAGTGGC (SEQ ID NO: 677) |
| TRCN0000107504 | CTGGAGTTCATAGCACCA | Forward: |
| | TAA (SEQ ID NO: 678) | CCGGCTGGAGTTCATAGCACCATAACTCGAGTTATGGTGCTAT |
| | | GAACTCCAGTTTTTG (SEQ ID NO: 679) |
| | | Reverse: |
| | | AATTCAAAAACTGGAGTTCATAGCACCATAACTCGAGTTATGG |
| | | TGCTATGAACTCCAG (SEQ ID NO: 680) |
| TRCN0000107501 | CCCGCTGAACATTATCAG | Forward: |
| | AAA (SEQ ID NO: 681) | CCGGCCCGCTGAACATTATCAGAAACTCGAGTTTCTGATAATGT |
| | | TCAGCGGGTTTTTG (SEQ ID NO: 682) |
| | | Reverse: |
| | | AATTCAAAAACCCGCTGAACATTATCAGAAACTCGAGTTTCTGA |
| | | TAATGTTCAGCGGG (SEQ ID NO: 683) |
| TRCN0000107503 | AGCCACTAAGTCAAGCT | Forward: |
| | GAAA (SEQ ID NO: 684) | CCGGAGCCACTAAGTCAAGCTGAAACTCGAGTTTCAGCTTGAC |
| | | TTAGTGGCTTTTTTG (SEQ ID NO: 685) |
| | | Reverse: |
| | | AATTCAAAAAAGCCACTAAGTCAAGCTGAAACTCGAGTTTCAG |
| | | CTTGACTTAGTGGCT (SEQ ID NO: 686) |
| TRCN0000107500 | CCAACTGGTCTAAGCAG | Forward: |
| | CATT (SEQ ID NO: 687) | CCGGCCAACTGGTCTAAGCAGCATTCTCGAGAATGCTGCTTAG |
| | | ACCAGTTGGTTTTTG (SEQ ID NO: 688) |
| | | Reverse: |
| | | AATTCAAAAACCAACTGGTCTAAGCAGCATTCTCGAGAATGCT |
| | | GCTTAGACCAGTTGG (SEQ ID NO: 689) |
| TRCN0000413154 | GAAACGAGGACACAATG | Forward: |
| | AAAT (SEQ ID NO: 690) | CCGGGAAACGAGGACACAATGAAATCTCGAGATTTCATTGTGT |
| | | CCTCGTTTCTTTTTG (SEQ ID NO: 691) |
| | | Reverse: |
| | | AATTCAAAAAGAAACGAGGACACAATGAAATCTCGAGATTTCA |
| | | TTGTGTCCTCGTTTC (SEQ ID NO: 692) |
| TRCN0000107502 | GCCACTAAGTCAAGCTG | Forward: |
| | AAAT (SEQ ID NO: 693) | CCGGGCCACTAAGTCAAGCTGAAATCTCGAGATTTCAGCTTGA |
| | | CTTAGTGGCTTTTTG (SEQ ID NO: 694) |
| | | Reverse: |
| | | AATTCAAAAAGCCACTAAGTCAAGCTGAAATCTCGAGATTTCA |
| | | GCTTGACTTAGTGGC (SEQ ID NO: 695) |
| TRCN0000107504 | CTGGAGTTCATAGCACCA | Forward: |
| | TAA (SEQ ID NO: 696) | CCGGCTGGAGTTCATAGCACCATAACTCGAGTTATGGTGCTAT |
| | | GAACTCCAGTTTTTG (SEQ ID NO: 697) |
| | | Reverse: |
| | | AATTCAAAAACTGGAGTTCATAGCACCATAACTCGAGTTATGG |
| | | TGCTATGAACTCCAG (SEQ ID NO: 698) |
| TRCN0000107501 | CCCGCTGAACATTATCAG | Forward: |
| | AAA (SEQ ID NO: 699) | CCGGCCCGCTGAACATTATCAGAAACTCGAGTTTCTGATAATGT |
| | | TCAGCGGGTTTTTG (SEQ ID NO: 700) |
| | | Reverse: |
| | | AATTCAAAAACCCGCTGAACATTATCAGAAACTCGAGTTTCTGA |
| | | TAATGTTCAGCGGG (SEQ ID NO: 701) |
| TRCN0000107503 | AGCCACTAAGTCAAGCT | Forward: |
| | GAAA (SEQ ID NO: 702) | CCGGAGCCACTAAGTCAAGCTGAAACTCGAGTTTCAGCTTGAC |
| | | TTAGTGGCTTTTTTG (SEQ ID NO: 703) |
| | | Reverse: |
| | | AATTCAAAAAAGCCACTAAGTCAAGCTGAAACTCGAGTTTCAG |
| | | CTTGACTTAGTGGCT (SEQ ID NO: 704) |
| TRCN0000107500 | CCAACTGGTCTAAGCAG | Forward: |
| | CATT (SEQ ID NO: 705) | CCGGCCAACTGGTCTAAGCAGCATTCTCGAGAATGCTGCTTAG |
| | | ACCAGTTGGTTTTTG (SEQ ID NO: 706) |
| | | Reverse: |
| | | AATTCAAAAACCAACTGGTCTAAGCAGCATTCTCGAGAATGCT |
| | | GCTTAGACCAGTTGG (SEQ ID NO: 707) |
| TRCN0000413154 | GAAACGAGGACACAATG | Forward: |
| | AAAT (SEQ ID NO: 708) | CCGGGAAACGAGGACACAATGAAATCTCGAGATTTCATTGTGT |
| | | CCTCGTTTCTTTTTG (SEQ ID NO: 709) |
| | | Reverse: |
| | | AATTCAAAAAGAAACGAGGACACAATGAAATCTCGAGATTTCA |
| | | TTGTGTCCTCGTTTC (SEQ ID NO: 710) |
| TRCN0000107502 | GCCACTAAGTCAAGCTG | Forward: |
| | AAAT (SEQ ID NO: 711) | CCGGGCCACTAAGTCAAGCTGAAATCTCGAGATTTCAGCTTGA |
| | | CTTAGTGGCTTTTTG (SEQ ID NO: 712) |
| | | Reverse: |
| | | AATTCAAAAAGCCACTAAGTCAAGCTGAAATCTCGAGATTTCA |
| | | GCTTGACTTAGTGGC (SEQ ID NO: 713) |
| TRCN0000107504 | CTGGAGTTCATAGCACCA | Forward: |
| | TAA (SEQ ID NO: 714) | CCGGCTGGAGTTCATAGCACCATAACTCGAGTTATGGTGCTAT |
| | | GAACTCCAGTTTTTG (SEQ ID NO: 715) |
| | | Reverse: |
| | | AATTCAAAAACTGGAGTTCATAGCACCATAACTCGAGTTATGG |
| | | TGCTATGAACTCCAG (SEQ ID NO: 716) |
| TRCN0000107501 | CCCGCTGAACATTATCAG | Forward: |
| | AAA (SEQ ID NO: 717) | CCGGCCCGCTGAACATTATCAGAAACTCGAGTTTCTGATAATGT |
| | | TCAGCGGGTTTTTG (SEQ ID NO: 718) |
| | | Reverse: |
| | | AATTCAAAAACCCGCTGAACATTATCAGAAACTCGAGTTTCTGA |
| | | TAATGTTCAGCGGG (SEQ ID NO: 719) |
| TRCN0000107503 | AGCCACTAAGTCAAGCT | Forward: |
| | GAAA (SEQ ID NO: 720) | CCGGAGCCACTAAGTCAAGCTGAAACTCGAGTTTCAGCTTGAC |
| | | TTAGTGGCTTTTTTG (SEQ ID NO: 721) |
| | | Reverse: |
| | | AATTCAAAAAAGCCACTAAGTCAAGCTGAAACTCGAGTTTCAG |
| | | CTTGACTTAGTGGCT (SEQ ID NO: 722) |
| TRCN0000107500 | CCAACTGGTCTAAGCAG | Forward: |
| | CATT (SEQ ID NO: 723) | CCGGCCAACTGGTCTAAGCAGCATTCTCGAGAATGCTGCTTAG |
| | | ACCAGTTGGTTTTTG (SEQ ID NO: 724) |
| | | Reverse: |
| | | AATTCAAAAACCAACTGGTCTAAGCAGCATTCTCGAGAATGCT |
| | | GCTTAGACCAGTTGG (SEQ ID NO: 725) |
| TRCN0000413154 | GAAACGAGGACACAATG | Forward: |
| | AAAT (SEQ ID NO: 726) | CCGGGAAACGAGGACACAATGAAATCTCGAGATTTCATTGTGT |
| | | CCTCGTTTCTTTTTG (SEQ ID NO: 727) |
| | | Reverse: |
| | | AATTCAAAAAGAAACGAGGACACAATGAAATCTCGAGATTTCA |
| | | TTGTGTCCTCGTTTC (SEQ ID NO: 728) |
| TRCN0000107502 | GCCACTAAGTCAAGCTG | Forward: |
| | AAAT (SEQ ID NO: 729) | CCGGGCCACTAAGTCAAGCTGAAATCTCGAGATTTCAGCTTGA |
| | | CTTAGTGGCTTTTTG (SEQ ID NO: 730) |
| | | Reverse: |
| | | AATTCAAAAAGCCACTAAGTCAAGCTGAAATCTCGAGATTTCA |
| | | GCTTGACTTAGTGGC (SEQ ID NO: 731) |
| TRCN0000107504 | CTGGAGTTCATAGCACCA | Forward: |
| | TAA (SEQ ID NO: 732) | CCGGCTGGAGTTCATAGCACCATAACTCGAGTTATGGTGCTAT |
| | | GAACTCCAGTTTTTG (SEQ ID NO: 733) |
| | | Reverse: |
| | | AATTCAAAAACTGGAGTTCATAGCACCATAACTCGAGTTATGG |
| | | TGCTATGAACTCCAG (SEQ ID NO: 734) |
| TRCN0000107501 | CCCGCTGAACATTATCAG | Forward: |
| | AAA (SEQ ID NO: 735) | CCGGCCCGCTGAACATTATCAGAAACTCGAGTTTCTGATAATGT |
| | | TCAGCGGGTTTTTG (SEQ ID NO: 736) |
| | | Reverse: |
| | | AATTCAAAAACCCGCTGAACATTATCAGAAACTCGAGTTTCTGA |
| | | TAATGTTCAGCGGG (SEQ ID NO: 737) |
| TRCN0000107503 | AGCCACTAAGTCAAGCT | Forward: |
| | GAAA (SEQ ID NO: 738) | CCGGAGCCACTAAGTCAAGCTGAAACTCGAGTTTCAGCTTGAC |
| | | TTAGTGGCTTTTTTG (SEQ ID NO: 738) |
| | | Reverse: |
| | | AATTCAAAAAAGCCACTAAGTCAAGCTGAAACTCGAGTTTCAG |
| | | CTTGACTTAGTGGCT (SEQ ID NO: 740) |
| TRCN0000107500 | CCAACTGGTCTAAGCAG | Forward: |
| | CATT (SEQ ID NO: 741) | CCGGCCAACTGGTCTAAGCAGCATTCTCGAGAATGCTGCTTAG |
| | | ACCAGTTGGTTTTTG (SEQ ID NO: 742) |
| | | Reverse: |
| | | AATTCAAAAACCAACTGGTCTAAGCAGCATTCTCGAGAATGCT |
| | | GCTTAGACCAGTTGG (SEQ ID NO: 742) |
| TRCN0000413154 | GAAACGAGGACACAATG | Forward: |
| | AAAT (SEQ ID NO: 743) | CCGGGAAACGAGGACACAATGAAATCTCGAGATTTCATTGTGT |
| | | CCTCGTTTCTTTTTG (SEQ ID NO: 744) |
| | | Reverse: |
| | | AATTCAAAAAGAAACGAGGACACAATGAAATCTCGAGATTTCA |
| | | TTGTGTCCTCGTTTC (SEQ ID NO: 745) |
| TRCN0000107502 | GCCACTAAGTCAAGCTG | Forward: |
| | AAAT (SEQ ID NO: 746) | CCGGGCCACTAAGTCAAGCTGAAATCTCGAGATTTCAGCTTGA |
| | | CTTAGTGGCTTTTTG (SEQ ID NO: 747) |
| | | Reverse: |
| | | AATTCAAAAAGCCACTAAGTCAAGCTGAAATCTCGAGATTTCA |
| | | GCTTGACTTAGTGGC (SEQ ID NO: 748) |
| TRCN0000107504 | CTGGAGTTCATAGCACCA | Forward: |
| | TAA (SEQ ID NO: 749) | CCGGCTGGAGTTCATAGCACCATAACTCGAGTTATGGTGCTAT |
| | | GAACTCCAGTTTTTG (SEQ ID NO: 750) |
| | | Reverse: |
| | | AATTCAAAAACTGGAGTTCATAGCACCATAACTCGAGTTATGG |
| | | TGCTATGAACTCCAG (SEQ ID NO: 751) |
| TRCN0000107501 | CCCGCTGAACATTATCAG | Forward: |
| | AAA (SEQ ID NO: 752) | CCGGCCCGCTGAACATTATCAGAAACTCGAGTTTCTGATAATGT |
| | | TCAGCGGGTTTTTG (SEQ ID NO: 753) |
| | | Reverse: |
| | | AATTCAAAAACCCGCTGAACATTATCAGAAACTCGAGTTTCTGA |
| | | TAATGTTCAGCGGG (SEQ ID NO: 754) |
| TRCN0000185775 | GATTGTTTCAACACAAGA | Forward: |
| | GTA (SEQ ID NO: 755) | CCGGGATTGTTTCAACACAAGAGTACTCGAGTACTCTTGTGTTG |
| | | AAACAATCTTTTTG (SEQ ID NO: 756) |
| | | Reverse: |
| | | AATTCAAAAAGATTGTTTCAACACAAGAGTACTCGAGTACTCTT |
| | | GTGTTGAAACAATC (SEQ ID NO: 757) |
| TRCN0000107503 | AGCCACTAAGTCAAGCT | Forward: |
| | GAAA (SEQ ID NO: 758) | CCGGAGCCACTAAGTCAAGCTGAAACTCGAGTTTCAGCTTGAC |
| | | TTAGTGGCTTTTTTG (SEQ ID NO: 759) |
| | | Reverse: |
| | | AATTCAAAAAAGCCACTAAGTCAAGCTGAAACTCGAGTTTCAG |
| | | CTTGACTTAGTGGCT (SEQ ID NO: 760) |
| TRCN0000107500 | CCAACTGGTCTAAGCAG | Forward: |
| | CATT (SEQ ID NO: 761) | CCGGCCAACTGGTCTAAGCAGCATTCTCGAGAATGCTGCTTAG |
| | | ACCAGTTGGTTTTTG (SEQ ID NO: 762) |
| | | Reverse: |
| | | AATTCAAAAACCAACTGGTCTAAGCAGCATTCTCGAGAATGCT |
| | | GCTTAGACCAGTTGG (SEQ ID NO: 763) |
| TRCN0000413154 | GAAACGAGGACACAATG | Forward: |
| | AAAT (SEQ ID NO: 764) | CCGGGAAACGAGGACACAATGAAATCTCGAGATTTCATTGTGT |
| | | CCTCGTTTCTTTTTG (SEQ ID NO: 765) |
| | | Reverse: |
| | | AATTCAAAAAGAAACGAGGACACAATGAAATCTCGAGATTTCA |
| | | TTGTGTCCTCGTTTC (SEQ ID NO: 766) |
| TRCN0000107502 | GCCACTAAGTCAAGCTG | Forward: |
| | AAAT (SEQ ID NO: 767) | CCGGGCCACTAAGTCAAGCTGAAATCTCGAGATTTCAGCTTGA |
| | | CTTAGTGGCTTTTTG (SEQ ID NO: 768) |
| | | Reverse: |
| | | AATTCAAAAAGCCACTAAGTCAAGCTGAAATCTCGAGATTTCA |
| | | GCTTGACTTAGTGGC (SEQ ID NO: 769) |
| TRCN0000107504 | CTGGAGTTCATAGCACCA | Forward: |
| | TAA (SEQ ID NO: 780) | CCGGCTGGAGTTCATAGCACCATAACTCGAGTTATGGTGCTAT |
| | | GAACTCCAGTTTTTG (SEQ ID NO: 781) |
| | | Reverse: |
| | | AATTCAAAAACTGGAGTTCATAGCACCATAACTCGAGTTATGG |
| | | TGCTATGAACTCCAG (SEQ ID NO: 782) |
| TRCN0000107501 | CCCGCTGAACATTATCAG | Forward: |
| | AAA (SEQ ID NO: 783) | CCGGCCCGCTGAACATTATCAGAAACTCGAGTTTCTGATAATGT |
| | | TCAGCGGGTTTTTG (SEQ ID NO: 784) |
| | | Reverse: |
| | | AATTCAAAAACCCGCTGAACATTATCAGAAACTCGAGTTTCTGA |
| | | TAATGTTCAGCGGG (SEQ ID NO: 785) |
| TRCN0000107503 | AGCCACTAAGTCAAGCT | Forward: |
| | GAAA (SEQ ID NO: 786) | CCGGAGCCACTAAGTCAAGCTGAAACTCGAGTTTCAGCTTGAC |
| | | TTAGTGGCTTTTTTG (SEQ ID NO: 787) |
| | | Reverse: |
| | | AATTCAAAAAAGCCACTAAGTCAAGCTGAAACTCGAGTTTCAG |
| | | CTTGACTTAGTGGCT (SEQ ID NO: 788) |
| TRCN0000107500 | CCAACTGGTCTAAGCAG | Forward: |
| | CATT (SEQ ID NO: 789) | CCGGCCAACTGGTCTAAGCAGCATTCTCGAGAATGCTGCTTAG |
| | | ACCAGTTGGTTTTTG (SEQ ID NO: 790) |
| | | Reverse: |
| | | AATTCAAAAACCAACTGGTCTAAGCAGCATTCTCGAGAATGCT |
| | | GCTTAGACCAGTTGG (SEQ ID NO: 791) |
| TRCN0000413154 | GAAACGAGGACACAATG | Forward: |
| | AAAT (SEQ ID NO: 792) | CCGGGAAACGAGGACACAATGAAATCTCGAGATTTCATTGTGT |
| | | CCTCGTTTCTTTTTG (SEQ ID NO: 793) |
| | | Reverse: |
| | | AATTCAAAAAGAAACGAGGACACAATGAAATCTCGAGATTTCA |
| | | TTGTGTCCTCGTTTC (SEQ ID NO: 794) |
| TRCN0000107502 | GCCACTAAGTCAAGCTG | Forward: |
| | AAAT (SEQ ID NO: 795) | CCGGGCCACTAAGTCAAGCTGAAATCTCGAGATTTCAGCTTGA |
| | | CTTAGTGGCTTTTTG (SEQ ID NO: 796) |
| | | Reverse: |
| | | AATTCAAAAAGCCACTAAGTCAAGCTGAAATCTCGAGATTTCA |
| | | GCTTGACTTAGTGGC (SEQ ID NO: 797) |
| TRCN0000107504 | CTGGAGTTCATAGCACCA | Forward: |
| | TAA (SEQ ID NO: 798) | CCGGCTGGAGTTCATAGCACCATAACTCGAGTTATGGTGCTAT |
| | | GAACTCCAGTTTTTG (SEQ ID NO: 799) |
| | | Reverse: |
| | | AATTCAAAAACTGGAGTTCATAGCACCATAACTCGAGTTATGG |
| | | TGCTATGAACTCCAG (SEQ ID NO: 800) |
| TRCN0000107501 | CCCGCTGAACATTATCAG | Forward: |
| | AAA (SEQ ID NO: 801) | CCGGCCCGCTGAACATTATCAGAAACTCGAGTTTCTGATAATGT |
| | | TCAGCGGGTTTTTG (SEQ ID NO: 802) |
| | | Reverse: |
| | | AATTCAAAAACCCGCTGAACATTATCAGAAACTCGAGTTTCTGA |
| | | TAATGTTCAGCGGG (SEQ ID NO: 803) |

In some embodiments, an AIM2 inflammasome inhibitor is a miRNA (miR) that inhibits the expression of AIM2, or an agonist of a miR that inhibits AIM2 expression. Exemplary miRs that inhibit AIM2 is miR-223 (Yang, Fan, et al. “MicroRNA-223 acts as an important regulator to Kupffer cells activation at the early stage of Con A-induced acute liver failure via AIM2 signaling pathway.” Cellular Physiology and Biochemistry 34.6 (2014): 2137-2152). Accordingly, an AIM2 inhibitor for use herein is miR-223 corresponding to any one of SEQ ID NO: 589-593.

A reconstituted in vitro AIM2 inflammasome in a cell-free system can be used as a tool to screen AIM2 inflammasome inhibitors according to the methods disclosed in Kaneko et al., 2015, or the methods disclosed in US application U52013/0158100A1, which is incorporated herein in its entirety by reference.

Inhibitors of Caspase-1

In some embodiments, an inflammasome antagonist inhibits caspase-1. In some embodiments, an inhibitor of caspase-1 for use in the methods and compositions is Belnacasan (VX-765). VX-765 is an orally absorbed prodrug of VRT-043198, a potent and selective inhibitor of caspases belonging to the ICE/caspase-1 subfamily, and has the formula as follows:

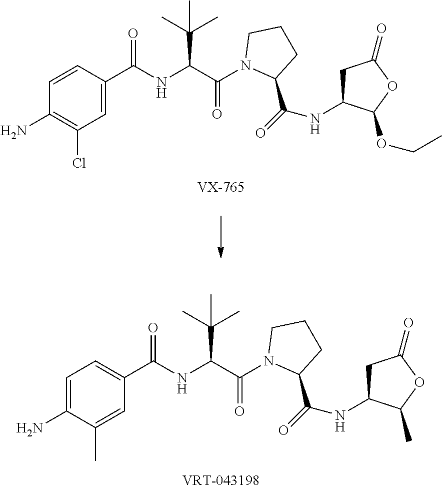

(see Wannamaker W. et al., 2007. (S)-1-((S)-2-{[1-(4-amino-3-chloro-phenyl)-methanoyl]-amino}-3,3-dimethyl-butanoyl)-pyrrolidine-2-carboxylic acid ((2R,3S)-2-ethoxy-5-oxo-tetrahydro-furan-3-yl)-amide (VX-765), an orally available selective interleukin (IL)-converting enzyme/caspase-1 inhibitor, exhibits potent anti-inflammatory activities by inhibiting the release of IL-1beta and IL-18. J Pharmacol Exp Ther. 321(2):509-16).

In some embodiments, the inhibitor of the caspase-1 is Z-VAD-FMK, which has the following structure:

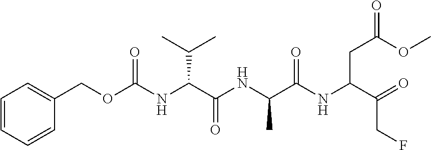 [SMILES: COC(=O)CC(NC(=O)[C@@H](C)NC(=O)[C@H](NC(=O)OCc1ccccc1)C(C)C)C(=O)CF]

and is a cell-permeable pan-caspase inhibitor and a potent inhibitor of caspase-1 activation in NLRP3-induced cells (Dostert C. et al., 2009. Malarial hemozoin is a Nalp3 inflammasome activating danger signal. PLoS One. 4(8):e6510). Z-VAD-FMK irreversibly binds to the catalytic site of caspase proteases (Slee E A. et al., 1996. Benzyloxycarbonyl-Val-Ala-Asp (OMe) fluoromethylketone (Z-VAD.FMK) inhibits apoptosis by blocking the processing of CPP32. Biochem J. 315 (Pt 1):21-4.)

In some embodiments, the inhibitor of the caspase-1 is Ac-YVAD-cmk, which has the following structure:

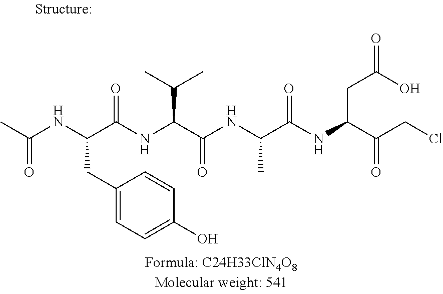 [SMILES: CC(=O)N[C@@H](Cc1ccc(O)cc1)C(=O)N[C@H](C(=O)N[C@@H](C)C(=O)N[C@@H](CC(=O)O)C(=O)CCl)C(C)C]

and is a caspase-1 inhibitor and a chloromethyl ketone tetrapeptide based on the target sequence in proIL-1β (YVHD). Ac-YVAD cmk was reported to block inflammasome activation, and hence to display anti inflammatory, anti apoptotic and anti pyroptotic effects.

In some embodiments, the inhibitor of the caspase-1 is Ac-YVAD-CHO, which has the following structure:

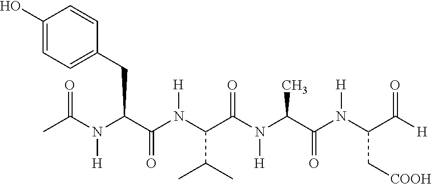 [SMILES: CC(=O)N[C@@H](Cc1ccc(O)cc1)C(=O)N[C@H](C(=O)N[C@@H](C)C(=O)N[C@H](C=O)CC(=O)O)C(C)C]

(Brenner, B., et al. 1998. Cell Death Differ. 5: 29-37. PMID: 10200443) Caspase-1 substrate (CAS 143305-11-7)

In some embodiments, the inhibitor of the caspase-1 is Parthenolide, which has the following structure:

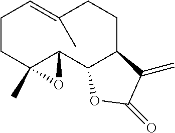 [SMILES: C=C1C(=O)O[C@H]2[C@H]1CC/C(C)=C/CC[C@@]1(C)O[C@@H]21]

Parthenolide, a sesquiterpene lactone derived from feverfew, is a known inhibitor of NF-κB activation, and also a direct inhibitor of caspase-1 and of multiple inflammasomes, including the NLRP3 and NLRP1 inflammasomes (Juliana C. et al., 2010. Anti-inflammatory compounds parthenolide and Bay 11-7082 are direct inhibitors of the inflammasome. J Biol Chem. 285(13):9792-802). Parthenolide directly inhibits the NLRP3 inflammasome by interfering with NLRP3 ATPase activity.

In some embodiments, the inhibitor of the caspase-1 is any one or a combination of: Pralnacasan (VX-740), which has the following structure:

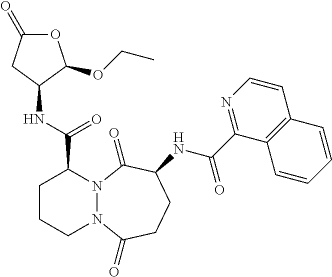 [SMILES: CCO[C@@H]1OC(=O)C[C@@H]1NC(=O)[C@@H]1CCCN2C(=O)CC[C@H](NC(=O)c3nccc4ccccc34)C(=O)N12]

Z-WEHD-FMK (also known as benzyloxycarbonyl-V-A-D-O-methyl fluoromethyl ketone).

In some embodiments, an inhibitor of caspase-1 is shikonin or acetylshikonin, where shikonin is:

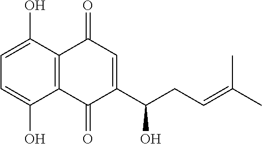 [SMILES: CC(C)=CC[C@@H](O)C1=CC(=O)c2c(O)ccc(O)c2C1=O]

and acetylshikonin is:

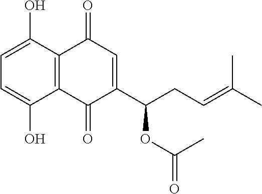 [SMILES: CC(=O)O[C@H](CC=C(C)C)C1=CC(=O)c2c(O)ccc(O)c2C1=O]

Shikonin is a highly lipophilic naphtoquinone found in the roots of Lithospermum erythrorhizon used for its pleiotropic effects in traditional Chinese medicine, and suppresses NLRP3 inflammasome activation (Zorman et al., PLOS One, 2016; 11 (7); e0159826.)

In some embodiments, the inhibitor of the caspase-1 may be a small molecule inhibitor, as one of skill in the art will appreciate. Non-limiting examples include cyanopropanate-containing molecules such as (S)-3-((S)-1-((S)-2-(4-amino-3-chlorobenzamido)-3,3-dimethylbutanoyl)pyrrolidine-2-carboxamido)-3-cyano-propanoic acid, as well as other small molecule caspase-1 inhibitors such as (S)-1-((S)-2-{[1-(4-amino-3-chloro-phenyl)-methanoyl]-amino }-3,3-dimethyl-butanoyl)-pyrrolidine-2-carboxylic acid ((2R,3 S)-2-ethoxy-5-oxo-tetrahydro-furan-3-yl)-amide. Such inhibitors may be chemically synthesized.

In some embodiments, the inhibitor of the caspase-1 may be a direct inhibitor of caspase-1 enzymatic activity, or may be an indirect inhibitor that inhibits initiation of inflammasome assembly or infiammasome signal propagation. Caspase-1 inhibitors for use in the present invention may be antioxidants, including reactive oxygen species (ROS) inhibitors. Examples of such caspase-1 inhibitors include, but are not limited to, flavonoids including flavones such as apigenin, luteolin, and diosmin; flavonols such as myricetin, fisetin and quercetin; flavanols and polymers thereof such as catechin, gallocatechin, epicatechin, epigallocatechin, epigallocatechin-3-gallate and theaflavin; isoflavone phytoestrogens; and stilbenoids such as resveratrol. Also included are phenolic acids and their esters such as gallic acid and salicyclic acid; terpenoids or isoprenoids such as andrographolide and parthenolide; vitamins such as vitamins A, C and E; vitamin cofactors such as co-enzyme Q10, manganese and iodide, other organic antioxidants such as citric acid, oxalic acid, phytic acid and alpha-lipoic acid, and Rhus verniciflua stokes extract. The caspase-1 inhibitor may be a combination of these compounds, for example, a combination of a-lipoic acid, co-enzyme Q10 and vitamin E, or a combination of a caspase 1 inhibitor(s) with another inflammasome inhibitor such as glyburide or a functionally equivalent precursor or derivative thereof.

Examples of dosages of some inflammasome inhibitors are as follows: apigenin (about 0.1-10 mg/kg), Luteolin (about 1-100 mg), Diosmin (about 100-900 mg), Myricetin (about 10-300 mg), Quercetin (about 10-1000 mg), Fisetin (1-200 mg/kg), Rhus verniciflua stokes extract (1−100 mg/kg), Catechin (about 50-500 mg), Gallocatechin (about 100-1000 mg), Epicatechin (about 0.1-10 mg/kg), Epigallocatechin (about 100-1000 mg), epigallocatechin-3-gallate (about 100-1000 mg), theaflavin (about 75-750 mg), isoflavone phytoestrogens (about 25-250 mg), resveratrol (about 100-1000 mg), andrographolide (about 100-500 mg), parthenolide (about 0.1-50 mg), vitamin A (about 5000-20000 IU), vitamin C (about 100-2000 mg), co-enzyme Q10 (about 30-500 mg), vitamin E (about 10-1000 IU), a-lipoic acid (about 10-1000 mg), co-enzyme Q10 (30-500 mg), manganese (about 1-100 mg), a-lipoic acid, co-enzyme Q10 and vitamin E (about 10-1000 mg, 30-500 mg, 10-1000 IU, respectively), glyburide (about 1-20 mg), and glyburide derivative lacking cyclohexylurea moiety (about 1-200 mg).

In some embodiments, the inhibitor of caspase-1 is any of the small molecule compounds described in U.S. Pat. Nos. 6,355,618; 6,632,962, 5,756,466 or International Applications: WO2001/042,216; WO2004/064,713, WO98/16502, WO 97/24339, EP623592, and Dolle et al., J. Med. Chem. 39, 2438 (1996); Dolle et al., J. Med. Chem. 40, 1941 (1997), the contents of each are herein incorporated by reference in their entireties. In some embodiments, an inhibitor of caspase-1 is a Nonpeptide inhibitors of caspase-1 have also been reported. U.S. Pat. No (Bemis et al.);

In some embodiments, the inhibitor of caspase-1 is an ICE (caspase-1) inhibitors having the structure:

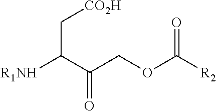

wherein R₁ is, inter alia, R₃CO—, R₃ is, inter alia, C₁-C₆ alkyl, aryl, heteroaryl, —(CHR)ₙ-aryl, and —(CHR)ₙ-heteroaryl, and R₂ is selected from various group. In some embodiments, the inhibitor of caspase-1 is an ICE (caspase-1) inhibitor having the structure:

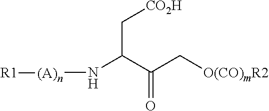

wherein R₁ includes aryl and heteroaryl; A is an amino acid; n is 0-4; m is 0 or 1; and R₂ is aryl. In some embodiments, the inhibitor of caspase-1 is an ICE (caspase-1) inhibitors having the structure:

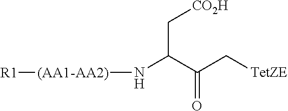

wherein R₁ includes aryl and heteroaryl; AA1 and AA2 are single bonds or amino acid residues; Tet represents a tetrazole ring; Z represents alkylene, alkenylene, 0, S etc.; and E represents H, alkyl, etc.

In some embodiments of the compositions and methods described herein, an inhibitor of caspase-1 is an RNA inhibitor of caspase-1, such as an siRNA specific for caspase-1. In some embodiments of the compositions and methods described herein, the RNA inhibitor of AIM2 is encoded by the ceDNA.

In some embodiments of the compositions and methods described herein, an inhibitor of caspase-1 is an RNA inhibitor of caspase-1, such as an siRNA specific for caspase-1. In some embodiments of the compositions and methods described herein, the RNA inhibitor of caspase-1 is encoded by the ceDNA. Examples of caspase-1 siRNA sequences encompassed for use in the kits and compositions herein are disclosed in WO2008/033,285; Keller, M., et al. Cell. 2008; 132(5): 818-831; Artlett, C. M., et al. Arthritis and Rheumatology. 2011 July; 63 (11): 3563-3574; Burdette, D., et al. J Gen Virology. 2012, 93: 235-246 which are incorporated herein in their entirety by reference. siRNA sequences to caspase-1 are also commercially available and are known to persons of ordinary skill

The human caspase-1 protein is encoded by the CASP1 gene comprising nucleic acid sequence NM_033292.3 (SEQ ID NO: 611), and the human caspase-1 protein has an amino acid of NP_150634.1 (SEQ ID NO: 612). Caspase-1 inhibitors further include antisense polynucleotides, which can be used to inhibit caspase-1 gene transcription and thereby inhibit caspase-1 and the downstream pathways of the NLRP3 inflammasome and AIM2 inflammasome. Polynucleotides that are complementary to a segment of a caspase-1-encoding polynucleotide (e.g., a polynucleotide as set forth in SEQ ID NO: 611) are designed to bind to caspase-1-encoding mRNA and to inhibit translation of such mRNA. Antisense polynucleotides can be encoded by a ceDNA vector as disclosed herein, and can optionally, be operatively linked to a tissue specific or inducible promoter as disclosed herein.

Inhibition of the caspase-1 or procaspase-1 mRNA can be by gene silencing RNAi molecules according to methods commonly known by a skilled artisan. For example, a gene silencing siRNA oligonucleotide duplexes targeted specifically to human caspase-1 (NM_033292.3) can readily be used to knockdown pro-caspase-1 expression. Caspase-1 mRNA can be successfully targeted using siRNAs; and other siRNA molecules may be readily prepared by those of skill in the art based on the known sequence of the target mRNA. Accordingly, in avoidance of any doubt, one of ordinary skill in the art can design nucleic acid inhibitors, such as RNAi (RNA silencing) agents to the nucleic acid sequence of NM_033292.3 which is as follows:

| (SEQ ID NO: 611) | |
|---|---|
| 1 | atactttcag tttcagtcac acaagaaggg aggagagaaa agccatggcc gacaaggtcc |
| 61 | tgaaggagaa gagaaagctg tttatccgtt ccatgggtga aggtacaata aatggcttac |
| 121 | tggatgaatt attacagaca agggtgctga acaaggaaga gatggagaaa gtaaaacgtg |
| 181 | aaaatgctac agttatggat aagacccgag ctttgattga ctccgttatt ccgaaagggg |
| 241 | cacaggcatg ccaaatttgc atcacataca tttgtgaaga agacagttac ctggcaggga |
| 301 | cgctgggact ctcagcagat caaacatctg gaaattacct taatatgcaa gactctcaag |
| 361 | gagtactttc ttcctttcca gctcctcagg cagtgcagga caacccagct atgcccacat |
| 421 | cctcaggctc agaagggaat gtcaagcttt gctccctaga agaagctcaa aggatatgga |
| 481 | aacaaaagtc ggcagagatt tatccaataa tggacaagtc aagccgcaca cgtcttgctc |
| 541 | tcattatctg caatgaagaa tttgacagta ttcctagaag aactggagct gaggttgaca |
| 601 | tcacaggcat gacaatgctg ctacaaaatc tggggtacag cgtagatgtg aaaaaaaatc |
| 661 | tcactgcttc ggacatgact acagagctgg aggcatttgc acaccgccca gagcacaaga |
| 721 | cctctgacag cacgttcctg gtgttcatgt ctcatggtat tcgggaaggc atttgtggga |
| 781 | agaaacactc tgagcaagtc ccagatatac tacaactcaa tgcaatcttt aacatgttga |
| 841 | ataccaagaa ctgcccaagt ttgaaggaca aaccgaaggt gatcatcatc caggcctgcc |
| 901 | gtggtgacag ccctggtgtg gtgtggttta aagattcagt aggagtttct ggaaacctat |
| 961 | ctttaccaac tacagaagag tttgaggatg atgctattaa gaaagcccac atagagaagg |
| 1021 | attttatcgc tttctgctct tccacaccag ataatgtttc ttggagacat cccacaatgg |
| 1081 | gctctgtttt tattggaaga ctcattgaac atatgcaaga atatgcctgt tcctgtgatg |
| 1141 | tggaggaaat tttccgcaag gttcgatttt catttgagca gccagatggt agagcgcaga |
| 1201 | tgcccaccac tgaaagagtg actttgacaa gatgtttcta cctcttccca ggacattaaa |
| 1261 | ataaggaaac tgtatgaatg tctgtgggca ggaagtgaag agatccttct gtaaaggttt |
| 1321 | ttggaattat gtctgctgaa taataaactt ttttgaaata ataaatctgg tagaaaaatg |
| 1381 | aaaaaaaaaa aaa |

In some embodiments, a caspase-1 inhibitor is a RNAi that is complementary to a RNAi target sequence in the NM_033292.3 (SEQ ID NO: 611); also referred to as NCBI gene 834 (CASP1). Current wild type transcripts for caspase-1 include: NM_001223.4, NM_001257118.2, NM_001257119.2, NM_033292.3 (SEQ ID NO: 611), NM_033293.3, NM_033294.3, NM_033295.3, XM_017018393.1, XM_017018394.1, XM_017018395.1, XM_017018396.1. A RNAi agent that inhibits caspase-1 can be a nucleic acid that is complementary to between 17-21 consecutive bases of SEQ ID NO: 613-619, shown Table 5E.

TABLE 5E
Target sequences for RNAi for inhibition of
caspase-1:

| Target Sequence | SEQ ID NO: | Clone ID |
|---|---|---|
| CACACGTCTTGCTCTCATTAT | 613 | TRCN0000003504 |
| CTACAACTCAATGCAATCTTT | 614 | TRCN0000003503 |
| CCAGATATACTACAACTCAAT | 615 | TRCN0000003502 |
| GAAGAGTTTGAGGATGATGCT | 616 | TRCN0000010796 |
| CCATGGGTGAAGGTACAATAA | 617 | TRCN0000118461 |
| GCTTTGATTGACTCCGTTATT | 618 | TRCN0000118459 |
| GAAGGTACAATAAATGGCTTA | 619 | TRCN0000118460 |

In some embodiments, a caspase-1 inhibitor is a siRNA agent, Exemplary siRNA sequences which inhibit caspase-1 are shown in Table 5F.

TABLE 5F
Exemplary siRNA which inhibit caspase-1

| Transcript | Clone ID | Target Seq | Forward and Reverse Oligo Sequences |
|---|---|---|---|
| NM_0033294.3 | TRCN0000003504 | CACACGTCTTGCTCTCA | Forward: |
| | | TTAT (SEQ ID NO: | CCGGCACACGTCTTGCTCTCATTATCTCGAGATAA |
| | | 620) | TGAGAGCAAGACGTGTGTTTTTG (SEQ ID NO: |
| | | | 621) |
| | | | Reverse: |
| | | | AATTCAAAAACACACGTCTTGCTCTCATTATCTCG |
| | | | AGATAATGAGAGCAAGACGTGTG (SEQ ID NO: |
| | | | 622) |
| NM_033294.3 | TRCN0000003503 | CTACAACTCAATGCAA | Forward: |
| | | TCTTT (SEQ ID NO: | CCGGCTACAACTCAATGCAATCTTTCTCGAGAAAG |
| | | 623) | ATTGCATTGAGTTGTAGTTTTTG (SEQ ID NO: |
| | | | 624) |
| | | | Reverse: |
| | | | AATTCAAAAACTACAACTCAATGCAATCTTTCTCG |
| | | | AGAAAGATTGCATTGAGTTGTAG (SEQ ID NO: |
| | | | 625) |
| NM_033294.3 | TRCN0000003502 | CCAGATATACTACAAC | Forward: |
| | | TCAAT (SEQ ID NO: | CCGGCCAGATATACTACAACTCAATCTCGAGATTG |
| | | 626) | AGTTGTAGTATATCTGGTTTTTG (SEQ ID NO: |
| | | | 627) |
| | | | Reverse: |
| | | | AATTCAAAAACCAGATATACTACAACTCAATCTCG |
| | | | AGATTGAGTTGTAGTATATCTGG (SEQ ID NO: |
| | | | 628) |
| NM_033294.3 | TRCN0000010795 | TGTATGAATGTCTGCT | Forward: |
| | | GGGCA (SEQ ID NO: | CCGGTGTATGAATGTCTGCTGGGCACTCGAGTGC |
| | | 629) | CCAGCAGACATTCATACATTTTTG (SEQ ID NO: |
| | | | 630) |
| | | | Reverse: |
| | | | AATTCAAAAATGTATGAATGTCTGCTGGGCACTC |
| | | | GAGTGCCCAGCAGACATTCATACA (SEQ ID NO: |
| | | | 631) |
| NM_033294.3 | TRCN0000139687 | CAAGGACCTGAAGGA | Forward: |
| | | GAAGAA (SEQ ID NO. | CCGGCAAGGACCTGAAGGAGAAGAACTCGAGTT |
| | | 632) | CTTCTCCTTCAGGTCCTTGTTTTTG (SEQ ID NO: |
| | | | 633) |
| | | | Reverse: |
| | | | AATTCAAAAACAAGGACCTGAAGGAGAAGAACTC |
| | | | GAGTTCTTCTCCTTCAGGTCCTTG (SEQ ID NO: |
| | | | 634) |
| NM_033294.3 | TRCN000013836 | CAATGTCTGTGGGAGG | Forward: |
| | | AAGAA (SEQ ID NO: | CCGGCAATGTCTGTGGGAGGAAGAACTCGAGTTC |
| | | 635) | TTCCTCCCACAGACATTGTTTTTG (SEQ ID NO: |
| | | | 636) |
| | | | Reverse: |
| | | | AATTCAAAAACAATGTCTGTGGGAGGAAGAACTC |
| | | | GAGTTCTTCCTCCCACAGACATTG (SEQ ID NO: |
| | | | 637) |
| NM_033294.3 | TRCN0000072917 | CAAGGTCCTGTAGGGA | Forward: |
| | | GAAGA (SEQ ID NO: | CCGGCAAGGTCCTGTAGGGAGAAGACTCGAGTCT |
| | | 638) | TCTCCCTACAGGACCTTGTTTTTG (SEQ ID NO: |
| | | | 639) |
| | | | Reverse: |
| | | | AATTCAAAAACAAGGTCCTGTAGGGAGAAGACTC |
| | | | GAGTCTTCTCCCTACAGGACCTTG (SEQ ID NO: |
| | | | 640) |
| NM_033294.3 | TRCN0000233250 | CAAGGTCCTGTAGGGA | Forward: |
| | | GAAGA (SEQ ID NO: | CCGGCAAGGTCCTGTAGGGAGAAGACTCGAGTCT |
| | | 641) | TCTCCCTACAGGACCTTGTTTTTG (SEQ ID NO: |
| | | | 642) |
| | | | Reverse: |
| | | | AATTCAAAAACAAGGTCCTGTAGGGAGAAGACTC |
| | | | GAGTCTTCTCCCTACAGGACCTTG (SEQ ID NO: |
| | | | 643) |
| NM_033294.3 | TRCN0000321071 | ACAAGCCCAAGGTGAT | Forward: |
| | | CATTA (SEQ ID NO: | CCGGACAAGCCCAAGGTGATCATTACTCGAGTAA |
| | | 644) | TGATCACCTTGGGCTTGTTTTTTG (SEQ ID NO: |
| | | | 645) |
| | | | Reverse: |
| | | | AATTCAAAAAACAAGCCCAAGGTGATCATTACTC |
| | | | GAGTAATGATCACCTTGGGCTTGT (SEQ ID NO: |
| | | | 646) |
| NM_033294.3 | TRCN0000125361 | CAAGGACTTGAAGGA | Forward: |
| | | GAAGAA (SEQ ID NO. | CCGGCAAGGACTTGAAGGAGAAGAACTCGAGTT |
| | | 647) | CTTCTCCTTCAAGTCCTTGTTTTTG (SEQ ID NO: |
| | | | 648) |
| | | | Reverse: |
| | | | AATTCAAAAACAAGGACTTGAAGGAGAAGAACTC |
| | | | GAGTTCTTCTCCTTCAAGTCCTTG (SEQ ID NO: |
| | | | 649) |
| NM_033294.3 | TRCN0000006653 | CCCAAGTTTGAAGTAC | Forward: |
| | | AAGTA (SEQ ID NO: | CCGGCCCAAGTTTGAAGTACAAGTACTCGAGTAC |
| | | 650) | TTGTACTTCAAACTTGGGTTTTTG (SEQ ID NO: |
| | | | 651) |
| | | | Reverse: |
| | | | AATTCAAAAACCCAAGTTTGAAGTACAAGTACTCG |
| | | | AGTACTTGTACTTCAAACTTGGG (SEQ ID NO: |
| | | | 652) |
| NM_033294.3 | TRCN0000058747 | CCCAGGACATGATAAT | Forward: |
| | | AAGAT (SEQ ID NO: | CCGGCCCAGGACATGATAATAAGATCTCGAGATC |
| | | 653) | TTATTATCATGTCCTGGGTTTTTG (SEQ ID NO: |
| | | | 654) |
| | | | Reverse: |
| | | | AATTCAAAAACCCAGGACATGATAATAAGATCTC |
| | | | GAGATCTTATTATCATGTCCTGGG (SEQ ID NO: |
| | | | 655) |
| NM_033294.3 | TRCN0000153291 | GAATTTGACAGTTTCCT | Forward: |
| | | GCCA (SEQ ID NO: | CCGGGAATTTGACAGTTTCCTGCCACTCGAGTGG |
| | | 656) | CAGGAAACTGTCAAATTCTTTTTG (SEQ ID NO: |
| | | | 657) |
| | | | Reverse: |
| | | | AATTCAAAAAGAATTTGACAGTTTCCTGCCACTCG |
| | | | AGTGGCAGGAAACTGTCAAATTC (SEQ ID NO: |
| | | | 658) |
| NM_033294.3 | TRCN0000073644 | CCCAAGTTTGAGGTCA | Forward: |
| | | AAGTT (SEQ ID NO: | CCGGCCCAAGTTTGAGGTCAAAGTTCTCGAGAAC |
| | | 659) | TTTGACCTCAAACTTGGGTTTTTG (SEQ ID NO: |
| | | | 660) |
| | | | Reverse: |
| | | | AATTCAAAAACCCAAGTTTGAGGTCAAAGTTCTCG |
| | | | AGAACTTTGACCTCAAACTTGGG (SEQ ID NO: |
| | | | 661) |
| NM_033294.3 | TRCN0000038805 | CGACAAGATGTTCTCC | Forward: |
| | | CTCAA (SEQ ID NO: | CCGGCGACAAGATGTTCTCCCTCAACTCGAGTTGA |
| | | 662) | GGGAGAACATCTTGTCGTTTTTG (SEQ ID NO: |
| | | | 663) |
| | | | Reverse: |
| | | | AATTCAAAAACGACAAGATGTTCTCCCTCAACTCG |
| | | | AGTTGAGGGAGAACATCTTGTCG (SEQ ID NO: |
| | | | 664) |

In some embodiments, a caspase-1 inhibitor is a siRNA, thereby inhibiting the mRNA of caspase-1 (or the pro-caspase-1 proprotein) thereby inhibiting the downstream pathways of the NLRP3 inflammasome and/or AIM2 inflammasome. In some embodiments, a caspase-1 inhibitor is GAA GGC CCA UAU AGA GAA A (SEQ ID NO: 904; sequence of sense strand is shown) which inhibits human caspase-1 expression or a fragment or a homologue thereof of at least 50%, or at least 60% or at least 70% or at least 80% or at least 90% identical thereto. Examples of caspase-1 siRNA sequences encompassed for use in the kits and compositions herein are disclosed in WO2008/033285 or US application US20090280058, Keller, M., et al. Cell. 2008; 132(5): 818-831; Artlett, C. M., et al. Arthritis and Rheumatology. 2011 July; 63 (11): 3563-3574; Burdette, D., et al. J Gen Virology. 2012, 93: 235-246; which are incorporated herein in their entirety by reference.

Custom siRNAs to NLRP3, AIM2 and caspase-1 can be generated on order from Dharmacon Research, Inc., Lafayette, Colo. Other sources for custom siRNA preparation include Xeragon Oligonucleotides, Huntsville, Ala. and Ambion of Austin, Tex. Alternatively, siRNAs can be chemically synthesized using ribonucleoside phosphoramidites and a DNA/RNA synthesizer. In some embodiments, a RNAi or siRNAs NLRP3, AIM2 and caspase-1 can be encoded in ceDNAs as disclosed herein.

In some embodiments, the inhibitor of caspase-1 is a Caspase-1 substrate (CAS 143305-11-7) having the structure of:

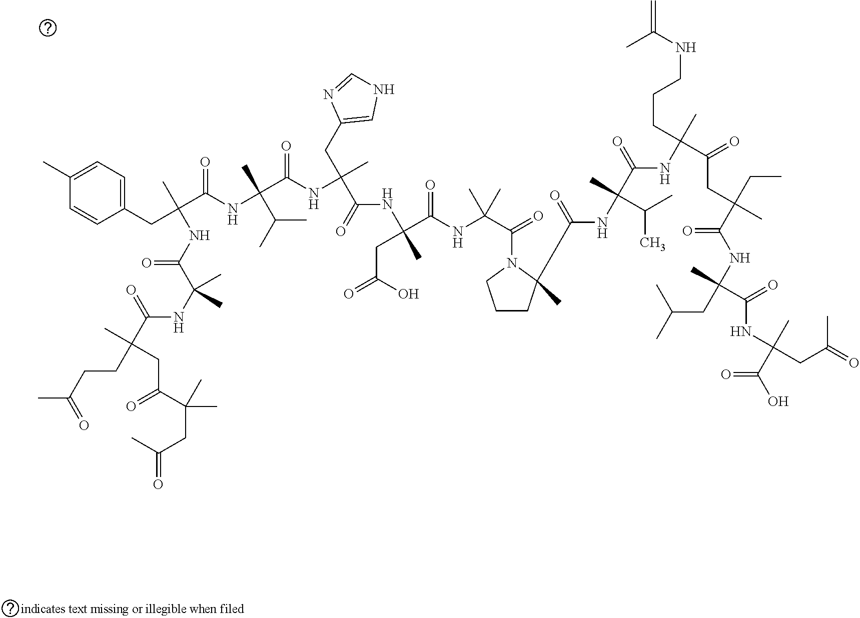

and which has the Sequence as follows: Asn-Glu-Ala-Tyr-Val-His-Asp-Ala-Pro-Val-Arg-Ser-Leu-Asn (SEQ ID NO: 538). In some embodiments of the compositions and methods described herein, an inhibitor of caspase-1 is encoded by a ceDNA being administered to a subject (including, e.g. subsequent delivery of ceDNA). In some embodiments of the compositions and methods described herein, an inhibitor of caspase-1 encoded by a ceDNA being administered to a subject is a caspase-1 substrate (SEQ ID NO: 538).

RNAi can be designed to target various mRNAs. A general strategy for designing RNAi, e.g., siRNAs comprises beginning with an AUG stop codon and then scanning the length of the desired cDNA target for AA dinucleotide sequences. The 3′ 19 nucleotides adjacent to the AA sequences were recorded as potential siRNA target sites. The potential target sites were then compared to the appropriate genome database, so that any target sequences that have significant homology to non-target genes could be discarded. Multiple target sequences along the length of the gene were located, so that target sequences were derived from the 3′, 5′ and medial portions of the mRNA. Negative control siRNAs were generated using the same nucleotide composition as the subject siRNA, but scrambled and checked so as to lack sequence homology to any genes of the cells being transfected. (Elbashir, S. M., et al., 2001, Nature, 411, 494-498; Ambion siRNA Design Protocol, at www.ambion.com).

Target sequences can be 17-25 bases long, and optimally 21 bases long, beginning with AA. RNAi or siRNA which bind the target sequences were modified with a thiol group at the 5 C6 carbon on one strand.

### VII. Methods of Use

A ceDNA vector for expression of ane.g. inhibitor of the immune response (e.g., the innate immune response) as disclosed herein can also be used in a method for the delivery of a nucleotide sequence of interest (e.g., encoding aninhibitor of the innate immune response) to a target cell (e.g., a host cell). The method may in particular be a method for delivering an inhibitor of the immune response (e.g., the innate immune response) to a cell of a subject in need thereof and treating an immune disorder, or to reduce or suppress the innate immune system. The invention allows for the in vivo expression of an inhibitor of the immune response (e.g., the innate immune response) encoded in the ceDNA vector in a cell in a subject such that therapeutic effect of the expression of an inflammasome antagonist occurs. These results are seen with both in vivo and in vitro modes of ceDNA vector delivery.

In addition, the invention provides a method for the delivery of inhibitor of the immune response (e.g., the innate immune response) e.g. in a cell of a subject in need thereof, comprising multiple administrations of the ceDNA vector of the invention encoding said inflammasome antagonist. Since the ceDNA vector of the invention does not induce an immune response like that typically observed against encapsidated viral vectors, such a multiple administration strategy will likely have greater success in a ceDNA-based system. The ceDNA vector are administered in sufficient amounts to transfect the cells of a desired tissue and to provide sufficient levels of gene transfer and expression of the inhibitor of the immune response (e.g., the innate immune response) e.g. without undue adverse effects. Conventional and pharmaceutically acceptable routes of administration include, but are not limited to, retinal administration (e.g., subretinal injection, suprachoroidal injection or intravitreal injection), intravenous (e.g., in a liposome formulation), direct delivery to the selected organ (e.g., any one or more tissues selected from: liver, kidneys, gallbladder, prostate, adrenal gland, heart, intestine, lung, and stomach), intramuscular, and other parental routes of administration. Routes of administration may be combined, if desired.

Delivery of a ceDNA vector for expression of e.g. inhibitor of the immune response (e.g., the innate immune response) as described herein is not limited to delivery of the expressed inhibitor. For example, conventionally produced (e.g., using a cell-based production method (e.g., insect-cell production methods) or synthetically produced ceDNA vectors as described herein may be used with other delivery systems provided to provide a portion of the gene therapy. One non-limiting example of a system that may be combined with the ceDNA vectors in accordance with the present disclosure includes systems which separately deliver one or more co-factors or immune suppressors for effective gene expression of the ceDNA vector expressing the inhibitor.

The invention also provides for a method of suppressing an immune response, e.g., innate immune response in a subject comprising introducing into a target cell in need thereof (in particular a muscle cell or tissue) of the subject a therapeutically effective amount of a ceDNA vector, optionally with a pharmaceutically acceptable carrier. While the ceDNA vector can be introduced in the presence of a carrier, such a carrier is not required. The ceDNA vector selected comprises a nucleotide sequence encoding an inhibitor of the immune response (e.g., the innate immune response) e.g. useful for treating or suppressing the immune system. In particular, the ceDNA vector may comprise a desired an inflammasome antagonist sequence operably linked to control elements capable of directing transcription of the desired inflammasome antagonist encoded by the exogenous DNA sequence when introduced into the subject. The ceDNA vector can be administered via any suitable route as provided above, and elsewhere herein.

The compositions and vectors provided herein can be used to deliver inhibitor of the immune response (e.g., the innate immune response) e.g. for various purposes. In some embodiments, the transgene encodes an inhibitor of the immune response (e.g., the innate immune response) that is intended to be used for research purposes, e.g., to create a somatic transgenic animal model harboring the transgene, e.g., to study the function of an inhibitor of the immune response (e.g., the innate immune response). In another example, the transgene encodes an inhibitor of the immune response (e.g., the innate immune response) that is intended to be used to create an animal model of a suppressed immune system or immunocompromised subject. In some embodiments, the encoded inhibitor of the immune response (e.g., the innate immune response) is useful for the treatment or prevention of an elevated immune responses or elevated innate immune state in a subject, e.g., in response to gene therapy or similar, in a mammalian subject. The inhibitor of the immune response (e.g., the innate immune response) can be transferred (e.g., expressed in) to a patient in a sufficient amount to reduce or prevent elevated immune responses in the subject.

A ceDNA vector is not limited to one species of ceDNA vector. As such, in another aspect, multiple ceDNA vectors expressing different proteins or the same inhibitors of the immune response (e.g., the innate immune response) e.g. but operatively linked to different promoters or cis-regulatory elements can be delivered simultaneously or sequentially to the target cell, tissue, organ, or subject. Therefore, this strategy can allow for the gene therapy or gene delivery of multiple an inflammasome antagonists simultaneously. It is also possible to separate different portions of an inhibitor into separate ceDNA vectors (e.g., different domains and/or co-factors required for functionality of an inhibitor of the immune response (e.g., the innate immune response) e.g. which can be administered simultaneously or at different times, and can be separately regulatable, thereby adding an additional level of control of expression of one or more inhibitors. Delivery can also be performed multiple times and, importantly for gene therapy in the clinical setting, in subsequent increasing or decreasing doses, given the lack of an anti-capsid host immune response due to the absence of a viral capsid. It is anticipated that no anti-capsid response will occur as there is no capsid.

The invention also provides for a method of suppressing an immune response, e.g., an innate immune response in a subject comprising introducing into a target cell in need thereof (in particular a muscle cell or tissue) of the subject a therapeutically effective amount of a ceDNA vector as disclosed herein, optionally with a pharmaceutically acceptable carrier. While the ceDNA vector can be introduced in the presence of a carrier, such a carrier is not required. The ceDNA vector implemented comprises a nucleotide sequence of interest, e.g., an inhibitor of the immune response useful for suppressing the innate immune system, or reducing an elevated immune state in a subject. In particular, the ceDNA vector may comprise a desired exogenous DNA sequence operably linked to control elements capable of directing transcription of the desired polypeptide, protein, or oligonucleotide encoded by the exogenous DNA sequence when introduced into the subject. The ceDNA vector can be administered via any suitable route as provided above, and elsewhere herein.

### Ex Vivo Treatment

In some embodiments, cells are removed from a subject, a ceDNA vector for expression of an inhibitor of the immune response (e.g., the innate immune response) e.g. as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of inhibitor of the immune response (e.g., the innate immune response) e.g. as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

In some embodiments, a ceDNA vector for expression of inhibitor of the immune response (e.g., the innate immune response) e.g. as disclosed herein can encode an inflammasome antagonist as described herein (sometimes called a transgene or heterologous nucleotide sequence) that is to be produced in a cell in vitro, ex vivo, or in vivo. For example, in contrast to the use of the ceDNA vectors described herein in a method of treatment as discussed herein, in some embodiments a ceDNA vector for expression of inhibitor of the immune response (e.g., the innate immune response) may be introduced into cultured cells and the expressed inflammasome antagonist isolated from the cells, e.g., for the production of antibodies and fusion proteins. In some embodiments, the cultured cells comprising a ceDNA vector for expression of inhibitor of the immune response (e.g., the innate immune response) as disclosed herein can be used for commercial production of antibodies or fusion proteins, e.g., serving as a cell source for small or large scale biomanufacturing of antibodies or fusion proteins. In alternative embodiments, a ceDNA vector for expression of an inhibitor of the immune response (e.g., the innate immune response) as disclosed herein is introduced into cells in a host non-human subject, for in vivo production of antibodies or fusion proteins, including small scale production as well as for commercial large scale inflammasome antagonist production.

The ceDNA vectors for expression of an inhibitor of the immune response (e.g., the innate immune response) as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### EXAMPLES

### Example 1: Constructing TTX-Plasmids

TTX format plasmids having the structure scheme shown in FIG. 4C (TTX-R) or FIG. 4D (TTX-L) were prepared. Examples of TTX-R and TTX-L plasmids are described in Table 6A below. The TTX-R and TTX-L plasmids differ by the position of a mutated AAV2 ITR sequence as shown in FIG. 4C and FIG. 4D, respectively. TTX-R plasmids (TTX-plasmid 1, 3, 5, and 7) were generated by molecular cloning disclosed herein to produce TTX-vectors. TTX-L plasmids (TTX-plasmid 2, 4, 6, and 8) for use in producing TTX-vectors (TTX-vector 2, 4, 6, 8). Each of the TTX-R plasmids comprise (a) a wild-type inverted terminal repeat (ITR) of AAV2; (b) an expression cassette and (c) a modified inverted terminal repeat (ITR) of AAV2, as illustrated in FIG. 4D.

ceDNA plasmids (i.e., plasmids comprising the ceDNA vector template used for later producing the ceDNA vector) can be constructed using known techniques to at least preferably provide the following as operatively linked components in the direction of transcription: a 5′ ITR (mutant or AAV wild type); control elements including a promoter, an exogenous DNA sequence of interest; a transcriptional termination region; and a 3′ ITR (mutant or wild type of the corresponding AAV ITR). Notably, the nucleotide sequences within the ITRs substantially replace the rep and cap coding regions. While rep sequences are ideally encoded by a helper plasmid or vector, it can alternatively be carried by the vector plasmid itself. In such cases, rep sequences are preferably located outside the region sandwiched between the ITRs, but can also be located within the region sandwiched between the ITRs. The desired exogenous DNA sequence is operably linked to control elements that direct the transcription or expression of an encoded polypeptide, protein, or oligonucleotide thereof in a cell, tissue, organ, or subject (i.e., in vitro, ex vivo, or in vivo). Such control elements can comprise control sequences normally associated with the selected gene. Alternatively, heterologous control sequences can be employed. Useful heterologous control sequences generally include those derived from sequences encoding mammalian or viral genes.

The desired exogenous DNA sequence in a ceDNA vector can be operably linked to control elements that direct the transcription or expression of an encoded polypeptide, protein, or oligonucleotide thereof in a cell, tissue, organ, or subject (i.e., in vitro, ex vivo, or in vivo). Such control elements can comprise control sequences normally associated with the selected gene. Alternatively, heterologous control sequences can be employed. Useful heterologous control sequences generally include those derived from sequences encoding mammalian or viral genes. Examples include, but are not limited to, promoters such as the SV40 early promoter; mouse mammary tumor virus LTR promoter; adenovirus major late promoter (Ad MLP); herpes simplex virus (HSV) promoters; a cytomegalovirus (CMV) promoter such as the CMV immediate early promoter region (CMVIE); a rous sarcoma virus (RSV) promoter; synthetic promoters; hybrid promoters; and the like. In addition, sequences derived from nonviral genes, such as the murine metallothionein gene, will also find use herein. ITR sequences of many AAV serotypes are known.

The expression cassette of each of the TTX plasmids (both TTX-R and TTX-L) includes the following between the ITR sequences: (i) an enhancer/promoter; (ii) a cloning site for a transgene; (iii) WHP Posttranscriptional Response Element (WPRE); and (iv) a poly-adenylation signal from bovine growth hormone gene (BGHpA). Unique restriction endonuclease recognition sites (R1-6) (e.g., see FIG. 4C and FIG. 4D) were also introduced between each component to facilitate the introduction of new genetic components into the specific sites in the construct. R3 and R4 enzyme sites are engineered into the cloning site to introduce an open reading frame of a transgene. These sequences were cloned into a pFastBac HT B plasmid obtained from ThermoFisher Scientific.

All TTX plasmids further comprise an exogenous sequence, which an open reading frame for a transgene (firefly Luciferase, or “Luc” or human factor IX, or “FIX”), were also generated by inserting the exogenous sequence into the cloning site. The structure of multiple examples of TTX plasmids provided in Table 6A were each constructed in the pattern of FIG. 4D (right sided mutated AAV ITR) or FIG. 4C (left sided mutated ITR). Each TTX plasmid included an enhancer/promoter and transgene (e.g., luciferase with various promoters or FIX with a CAG promoter), a post-translational regulatory element (WPRE) and a polyadenylation termination signal (BGH polyA) flanked by: (a) a mutated AAV2 inverted terminal repeat (ITR) polynucleotide sequence encoded in the plasmid on either the left (L) or the right (R) side of the expression cassette, and (b) a wild type (unmutated) AAV2 ITR sequence on opposite end of the expression cassette.

The TTX plasmids in Table 6A were constructed with the WPRE comprising SEQ ID NO: 8 and BGHpA comprising SEQ ID NO: 9 as components between the luciferase transgene and the right side ITR. In addition, each of the TTX plasmids (TTX-1 through TTX-10) also contained a R3/R4 cloning site (SEQ ID NO: 7) on either side of the Luciferase or factor IX (Padua FIX of SEQ ID NO: 12 or FIX of SEQ ID NO:11) ORF reporter sequence.

Referring to Table 6A:
“wt-L” refers to wild type AAV2 ITR encoded in the plasmid on the left side of the expression cassette (comprising the polynucleotide sequence of SEQ ID NO:51);
“wt-R” refers to wild type AAV2 ITR encoded in the plasmid on the right side of the expression cassette (comprising the polynucleotide sequence of SEQ ID NO:1);
“mut-L” refers to the mutated AAV2 ITR sequence provided in SEQ ID NO:52;
“mut-R” refers to the mutated AAV2 ITR sequence provided in SEQ ID NO:2;
“CAG” refers to the synthetic promoter constructed from (C) the cytomegalovirus immediate early enhancer and promoter elements, (A) the first exon and the first intron of the chicken beta-actin gene, (G) the splice acceptor of the rabbit beta-globin gene, of SEQ ID NO:3;
“AAT w/SV40 intr” refers to (human alpha 1-antitrypsin) AAT with 5V40 large T-antigen intron of SEQ ID NO:4; and
“hEF1-α” refers to human Elongation Factor-1 alpha (EF-1 alpha) of SEQ ID NO:6.

TABLE 6A

| Plasmid | ITR-L | Promoter | Transgene | ITR-R |
|---|---|---|---|---|
| TTX-1 | wt-L | CAG | Luciferase | mut-R |
| TTX-2 | mut-L | CAG | Luciferase | wt-R |
| TTX-3 | wt-L | AAT w/SV40 intr | Luciferase | mut-R |
| TTX-4 | mut-L | AAT w/SV40 intr | Luciferase | wt-R |
| TTX-5 | wt-L | LP1 w/SV40 intr | Luciferase | mut-R |
| TTX-6 | mut-L | LP1 w/SV40 intr | Luciferase | wt-R |
| TTX-7 | wt-L | hEF1-α | Luciferase | mut-R |
| TTX-8 | mut-L | hEF1-α | Luciferase | wt-R |
| TTX-9 | wt-L | CAG | Padua FIX | mut-R |
| TTX-10 | wt-L | CAG | FIX | mut-R |

TABLE 6B

| Plasmid | ITR-L | Promoter | Transgene | ITR-R |
|---|---|---|---|---|
| α (alpha) | wt-L | CAG | Luc | mut-R |
| β (beta) | wt-L | LP-1 β | FIX | mut-R |

Each construct in Table 6B contains a modified SV40 PolyA sequence (SEQ ID NO: 10), positioned in the 3′ untranslated region (UTR) between the Transgene and the mut-R ITR.
“LP-1β” refers to the LP-1β promoter (SEQ ID NO:16) which is the same as the LP-1 promoter (SEQ ID NO: 5) with 2 additional restriction enzyme sites.

In one embodiment, the vector polynucleotide (the ceDNA vector) comprises a pair of two different ITRs selected from the group consisting of: SEQ ID NO:1 and SEQ ID NO:52; and SEQ ID NO:2 and SEQ ID NO:51. In one embodiment of each of these aspects, the vector polynucleotide or the non-viral, capsid-free DNA vectors with covalently-closed ends comprises a pair of ITRs selected from the group consisting of: SEQ ID NO:101 and SEQ ID NO:102; SEQ ID NO:103, and SEQ ID NO:104, SEQ ID NO:105, and SEQ ID NO:106; SEQ ID NO:107, and SEQ ID NO:108; SEQ ID NO:109, and SEQ ID NO:110; SEQ ID NO:111, and SEQ ID NO:112; SEQ ID NO:113 and SEQ ID NO:114; and SEQ ID NO:115 and SEQ ID NO:116. In some embodiments, the ceDNA vectors do not have an ITR that comprises any sequence selected from SEQ ID NOs: 500-529.

### Example 2: Bacmid and Baculovirus for Generating Linear, Continuous, and Non-Encapsidated DNA Vectors

DH10Bac competent cells (MAX Efficiency® DH10Bac™ Competent Cells, Thermo Fisher, cat#10361012) were transformed with either the TTX or control plasmids following a protocol provided by the vendor available at their website (Thermo Fisher, found on the world wide web at https://www.thermofisher.com/order/catalog/product/10361012). Recombination between the plasmid and a baculovirus shuttle vector in the DH10Bac cells were induced to generate recombinant bacmids (“TTX-bacmids”). The recombinant bacmids were selected by a positive selection based on blue-white screening in E. coli (φ80dlacZΔM15 marker provides α-complementation of the β-galactosidase gene from the bacmid vector) on a bacterial agar plate containing X-gal and IPTG. White colonies were picked and cultured in 10 ml of media.

The recombinant bacmids (“TTX-bacmids”) were isolated from the E. coli and transfected into Sf9 or Sf21 insect cells using FugeneHD™ to produce infectious baculovirus. The adherent Sf9 or Sf21 insect cells were cultured in 50 ml of media in T25 flasks at 25° C. Four days later, culture medium (containing the PO virus) was removed from the cells, filtered through a 0.45 μm filter, and infectious recombinant baculovirus particles (“TTX-baculovirus” or “Comparative-baculovirus”) separating the baculovirus from the cells in the culture.

Optionally, the first generation of the baculovirus (P0) was amplified by infecting naïve Sf9 or Sf21 insect cells in 50 to 500 ml of media. Cells were cultured at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The TTX-baculovirus were collected and the infectious activity of the baculovirus was determined. Specifically, four×20 ml Sf9 cell cultures at 2.5E+6 cells/ml were treated with P1 baculovirus at the following dilutions, 1/1000, 1/10,000, 1/50,000, 1/100,000, and incubated. Infectivity was determined by the rate of cell diameters increase and cell cycle arrest, and change in cell viability every day for 4 to 5 days.

Rep 78 sequence (SEQ ID NO: 13) was operatively linked to IE1 promoter fragment (SEQ ID NO: 15) and then inserted into BamHI/KpnI restriction site of pFASTBAC™-Dual expression vector (ThermoFisher Catalog No: 10712024) so that Rep 78 sequence is linked to HSV TK poly A sequence on the 3′-end. The Rep 52 sequence (SEQ ID NO:14) was then cloned into the SalI-HindIII site of the vector to make the Rep52 sequence operatively linked to the pPH promoter on the 5′ and SV40 poly A sequence on the 3′. The resulting construct is referred to herein as “Rep-plasmid”.

The Rep-plasmid was transformed into the DH10Bac competent cells (MAX Efficiency® DH10BaC™ Competent Cells, Thermo Fisher, cat#10361012) following a protocol provided by the vendor available at their website (Thermo Fisher®, https://www.thermofisher.com/order/catalog/product/10361012). Recombination between the Rep-plasmid and a baculovirus shuttle vector in the DH10Bac cells were induced to generate recombinant bacmids (“Rep-bacmids”). The recombinant bacmids were selected by a positive selection based on blue-white screening in E. coli (φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (Kanamycin, Gentamicin, Tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 ml Sf9 cell cultures at 2.5×10⁶ cells/ml were treated with P1 baculovirus at the following dilutions, 1/1000, 1/10,000, 1/50,000, 1/100,000, and incubated. Infectivity was determined by the rate of cell diameters increase and cell cycle arrest, and change in cell viability every day for 4 to 5 days.

The Sf cell culture media containing either (1) TTX or α (alpha)-baculovirus, or (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The TTX or α (alpha)-vectors were isolated and purified from the cells using Qiagen Midi Plus purification protocol (Qiagen cat #12945, 0.2 mg of cell pellet mass processed per column).

Yields of DNA vectors (e.g., TTX vectors) produced and purified from the Sf9 insect cells were initially determined based on UV absorbance at 260 nm. Yields of various TTX-DNA vectors determined based on UV absorbance are provided below in Table 7.

TABLE 7

| | | Culture Parameters | | Estimated |
|---|---|---|---|---|
| | Culture | (Diameter in | Yield | Yield |
| Construct | Volume | micrometers) | (mg/L) | (pg/cell) |
| TTX-1 | 2x1L | Total: 6.02 × 10e6 | 15.8 | 5.23 |
| | | Viability: 53.3% | | |
| | | Diameter: 18.4 u | | |
| TTX-9 | 1L | Total: 6.65 × 10e6 | 4.8 | 0.88 |
| | | Viability: 81.8% | | |
| | | Diameter: 18.7 u | | |
| | 4L | Total: 2.45 × 10e6 | 5.9 | 3.5 |
| | | Viability: 74.5% | | |
| | | Diameter: 18.5 u | | |
| | 1L | Total: 4.92 × 10e6 | 4.0 | 1.0 |
| | | Viability: 84.4% | | |
| | | Diameter: 19.7 u | | |
| TTX-10 | 1L | Total: 5.55 × 10e6 | 6.5 | 1.3 |
| | | Viability: 77.4% | | |
| | | Diameter: 18.6 u | | |

### Example 3: Denaturing Gel Electrophoresis to Identify Production of ceDNA Vector

To demonstrate in a qualitative fashion that isolated DNA Vectors material is covalently close-ended as is required by definition, samples are digested with a restriction endonuclease identified by DNA vector sequence as having a single restriction site, preferably resulting in two cleavage products of unequal size (ex: 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA will resolve at 2× sizes (2000 bp and 4000 bp), as the two DNA strands are linked and are now unfolded and twice the length (though single stranded). Furthermore, digestion of monomeric, dimeric, and n-meric forms of the DNA vector will all resolve as the same size fragments due to the end-to-end linking of the multimeric DNA vector (see FIG. 5B).

As used herein, the phrase “Assay for the Identification of DNA vector by agarose gel electrophoresis under native gel and denaturing conditions” refers to the following assay. For restriction endonuclease, choose single cut enzyme to generate products of approximately ⅓× and ⅔× of the DNA vector length. This resolves the bands on both native and denaturing gels. Before denaturation, it is important to remove the buffer from the sample. The Qiagen PCR clean-up kit (Qiagen cat#28104) or desalting “spin columns,” e.g. GE HealthCare Ilustra™ MicroSpin™ G-25 columns (GE Healthcare cat #27532501) works well with the endonuclease digestion.
1. Digest DNA with appropriate restriction endonuclease(s)
2. Apply to Qiagen PCR clean-up kit, elute with dH2O (30 ul)
3. Add 4 ul of 10× denaturing solution (10×=0.5 M NaOH, 10 mM EDTA)
4. Add 6 ul of 10× gel loading solution (dye plus glycerol or ficoll, NOT buffered)
5. DNA ladders may be prepared without Qiagen kit by adding 10× denaturing solution to a final concentration of 4×.
6. Prepare 0.8-1.0% gel in H2O in microwave until boiling, let sit at ambient temperature for several minutes.
7. Pour into gel tray with comb and place in cold room to accelerate polymerization (2 hr)
8. Place tray into electrophoresis box and equilibrate with 1mMEDTA and 200 mM NaOH for 2 h with occasional agitation to ensure that the NaOH concentration is uniform in the gel and gel box.
9. Make 1 L of 1× denaturing solution (50 mM NaOH, 1 mM EDTA)
10. Pour sufficient volume into gel box to submerge gel to a depth of greater than 0.5 cm.
11. Large gels (15-20 cm)—Run gel overnight at 25V. medium gels (8-11 cm) run O/N @ 20V. Post Gel Run
12. Transfer gel to tray and wash with dH2O
13. Drain and neutralize gel in 1× TBE or TAE (20 min with gentle agitation)
14. Transfer gel to dH2O (or 1× TBE/TAE) with 1× SYBR Gold (20 min with gentle agitation) Thermo Fisher, SYBR® Gold Nucleic Acid Gel Stain (10,000× Concentrate in DMSO) Catalog number: 511494
15. Image gel with epifluorescent light (blue) or UV (312 nm)

Isolated DNA Vectors—vector are identified by agarose gel electrophoresis under native or denaturing condition as illustrated in FIG. 5 and FIG. 6. DNA vector generate multiple bands on native gels as provided in FIG. 5A. Each band can represent vectors having a different conformation in the native condition, e.g., continuous, non-continuous, monomeric, dimeric, etc.

Structures of the isolated DNA vector were further analyzed by digesting the DNA obtained from co-infected Sf9 cells (as described herein) with restriction endonucleases selected for a) the presence of only a single cut site within the DNA vector, and b) resulting fragments that were large enough to be seen clearly when fractionated on a 0.8% denaturing agarose gel (>800 bp).

Specifically, equal amounts (2 μg based on OD260) of TTX-plasmid and TTX-vector were digested at 37° C. for 1 hour with the restriction endonucleases. Following digestion, DNA vector material was isolated using a QIAquick column and eluted in water. Samples were denatured in denaturing solution (0.05M NaOH, 1 mM EDTA) while a 0.8% agarose gel made in water was pre-equilibrated for 2 hours in Equilibration Buffer (1 mM EDTA, 200 mM NaOH). Samples were then run on the gel overnight at 4° C. submerged in 1× Denaturing Solution (50 mM NaOH, 1 mM EDTA). The next day, the gel was washed, neutralized in TBE for 20 min, soaked in a 1× SYBR Gold water solution for 1 hour, and imaged under UV/Blue lighting.

The presence of the DNA vector is identified by the characteristic multi-band patterns initially on the native gel (primary and secondary bands spaced to indicate that the secondary band represents material at about twice the mass of the primary band), and then confirmed on a denatured gel by the characteristic multiband pattern illustrated on the right side of FIG. 5A. As illustrated in FIG. 5B, linear DNA vectors with a non-continuous structure and TTX-vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

FIG. 6 is an exemplary picture of an actual denaturing gel with TTX vectors 1 and 2, 3 and 4, 5 and 6 and 7 and 8 (all described in Table 1A above), with (+) or without (−) digestion by the endonuclease. Each TTX vector produced two bands (*) after the endonuclease reaction. Their two band sizes determined based on the size marker are provided on the bottom of the picture. The band sizes confirm that each of the TTX vectors has a continuous structure.

Contribution of TTX-plasmid to the UV absorbance was estimated by comparing fluorescent intensity of TTX-vector to a standard. For example, if based on UV absorbance 4 μg of TTX-vector was loaded on the gel, and the TTX-vector fluorescent intensity is equivalent to a 2 kb band which is known to be 1 μg, then there is 1 μg of TTX-vector. Thus, the TTX-vector is 25% of the total UV absorbing material. Band intensity on the gel is then plotted against the calculated input that band represents—for example, if the total TTX-vector is 8 kb, and the excised comparative band is 2 kb, then the band intensity would be plotted as 25% of the total input, which in this case would be 0.25 μg for 1.0 μg input. Using the TTX-plasmid titration to plot a standard curve, a regression line equation is then used to calculate the quantity of the TTX-vector band, which can then be used to determine the percent of total input represented by the TTX-vector, or percent purity (FIG. 7).

### Example 4: DNA Vectors Express Transgene Encoded Protein. In Vitro

SA wild-type cDNA sequence of human factor IX mRNA (“wtFIX”, SEQ ID NO: 11) or Padua variant of the cDNA sequence (“PaduaFIX”, SEQ ID NO: 12) was introduced into the cloning site of TTX-plasmid 1 to generate TTX-plasmid 1-wtFIX and TTX-plasmid 1-PaduaFIX, respectively. These plasmids were introduced into Sf9 insect cells and used to generate TTX-bacmid 1-wtFIX and TTX-bacmid 1-PaduaFIX, and TTX-baculovirus 1-wtFIX and TTX-baculovirus 1-PaduaFIX, respectively, using the methods described herein. In vitro protein expression from the TTX-plasmids and TTX-vectors was tested by transfecting HEK293 cells (2E+5 cells/well, 96 well plate) with 250 ng/well of (1) TTX-plasmid 1-wtFIX, (2) TTX-plasmid 1-PaduaFIX, (3) TTX-vector 1-wtFIX, (4) TTX-vector 1-PaduaFIX, (5) β (beta)-plasmid 1-wtFIX, or (6) β (beta)-vector 1-wtFIX, using Fugene6 transfection reagent (3:1 Fugene6:DNA). The result from the western blot analysis is provided in FIG. 8. FIX-antibody reaction revealed 55 kDa-bands which correspond to the mass of FIX proteins produced. The negative control lysates transfected with β (beta)-plasmid 1-wtFIX or β (beta)-vector 1-wtFIX did not produce a detectable amount of FIX protein. This result confirms that TTX-vector 1 can be used for effective transfer and expression of a therapeutic gene, such as a gene encoding human factor IX.

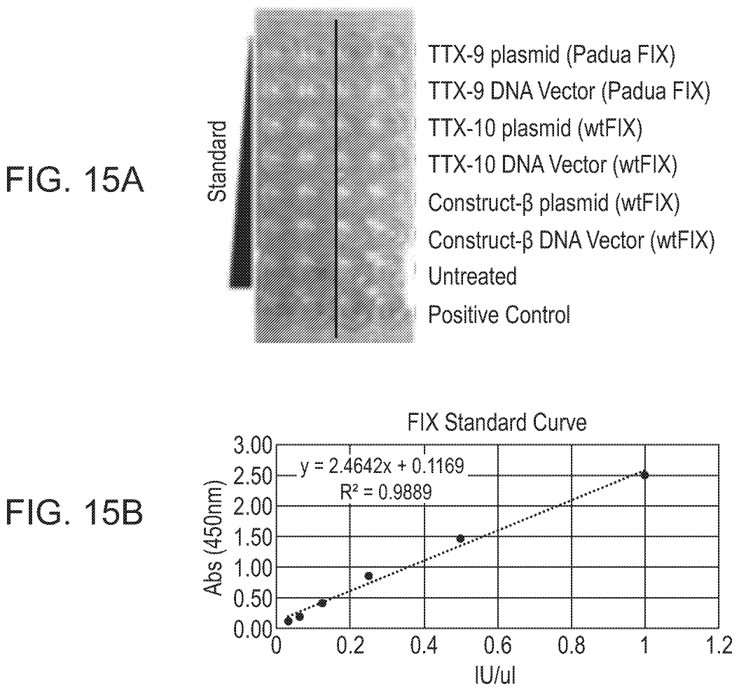

ELISA: Briefly, culture media from transfected cells was added in duplicate to anti-FIX antibody treated wells and incubated for 1 hour, followed by washing and incubation with a detecting antibody for 1 hour at room temperature. Samples were again washed, TMB substrate was added and developed for 10 minutes, stopped, and samples were immediately read for absorbance at 450 nm. An example of the samples after the TMB substrate reactions is provided in FIG. 15A and the concentration of FIX in each sample determined based on sample absorbance at 450 nm are provided in FIG. 15A. High-level expression of FIX protein from TTX-plasmid 1 and TTX-vector 1 was detected, while no significant expression of FIX was detected from β (Comparative)-plasmid or β (Comparative) vector.

This again confirms that TTX-vector 1 produced from TTX-plasmid 1, comprising from 5′ to 3′-WT-replicative polynucleotide sequence (SEQ ID NO: 51), CAG promoter (SEQ ID NO:3), R3/R4 cloning site (SEQ ID NO:7), WPRE (SEQ ID NO: 8), BGHpA (SEQ ID NO:9) and a modified replicative polynucleotide sequence (SEQ ID NO:2), is significantly more effective in inducing expression of a transgene compared to a (alpha)-vector 1 produced from a (alpha)-plasmid 1 which do not include the WPRE (SEQ ID NO: 8) and BGHpA (SEQ ID NO:9).

### Example 5: Preparing a ceDNA Co-Expressing Factor IX and a cGAS Inhibitor

Kaposi's sarcoma-associated herpesvirus protein ORF52 (SEQ ID NO: 882) or a variant thereof that inhibits cGAS, or a truncated cytoplasmic LANA isoform (LANAΔ161 or SEQ ID NO: 884) lacking amino acids 161-1162 of SEQ ID NO: 882) is operably linked to a promoter and inserted into the restriction cloning site R5 of TTX 9 or TTX 10 plasmid that encodes Factor IX transgene, as described in Example 1 and Example 4. A ceDNA is thus prepared that encodes both Factor IX and a cGAS inhibitor as described in Examples 2-3.

### Example 6: Confirming Expression of a cGAS Inhibitor Expressed by a ceDNA

Expression of a desired cGAS inhibitor co-expressed by a ceDNA, such as Kaposi's sarcoma-associated herpesvirus protein ORF52 (SEQ ID NO: 882) or a variant thereof that inhibits cGAS, or a truncated cytoplasmic LANA isoform (SEQ ID NO: 884), can be confirmed using HeLa cells and antibodies specific for the cGAS inhibitor, such as the antibody to ORF52 described in Li et al. (“Kaposi's sarcoma-associated herpesvirus inhibitor of cGAS (KicGAS) Encoded by ORF52, is an Abundant Tegument protein and Is Required for Production of Infectious Progeny Viruses,” J. Virol. 2016, 90(11): 5329). For example, HeLA cells are cultured and transient transfections of the constructs co-expressing the Factor IX and the desired cGAS inhibitor are performed using, for example, Fusegene6 transfection reagent (3:1; fusgene6:DNA). Western blot techniques and/or flow cytometry, as known to those of skill in the art, are used to detect expression of the cGAS inhibitor. The expression of Faxtor IX is confirmed as described in Example 4.

### Example 7: Preparing a ceDNA Co-Expressing Factor IX and a TLR-9 Inhibitor

Oligonucleotides that can form a hairpin structure comprising the following sequences, such as, (TCCTGGCGGGGAAGT, SEQ ID NO: 889), ODN-2114 (TCCTGGAGGGGAAGT, SEQ ID NO: 890), poly-G (GGGGGGGGGGGGGGGGGGGG, SEQ ID NO: 891), ODN-A151 (TTAGGGTTAGGGTTAGGGTTAGGG, SEQ ID NO: 892), G-ODN (CTCC-TATTGGGGGTTTCCTAT, SEQ ID NO: 893), IRS-869 (TCCTGGAGGGGTTGT, SEQ ID NO: 894), INH-1 (CCTGGATGGGAATTCCCATCCAGG, SEQ ID NO: 895), INH-4 (TTCCCATCCAGGCCTGGATGGGAA, SEQ ID NO: 896), (IRS-661 TGCTTGCAAGCTT-GCAAGCA, SEQ ID NO: 897), 4024 (TCCTGGATGGGAAGT, SEQ ID NO: 898), 4084F (CCTGGATGGGAA, SEQ ID NO: 899), INH-13 (CTTACCGCTGCACCTGGATGGGAA, SEQ ID NO: 900), INH-18 (CCTGGATGGGAACTTACCGCTGCA, SEQ ID NO: 901), and IRS-954 TGCTCCTGGAGGGGTTGT, SEQ ID NO: 902) are engineered to have sticky ends after annealing of 5′ to 3′ and complementary 3′ to 5′ strands such that they can be inserted by ligation into a preselected restriction cloning site, e.g. R5 or other site of TTX 9 or TTX 10 plasmid that encodes Factor IX transgene, as described in Example 1 and Example 4.

For example, oligos with appropriate restriction site are annealed by mixing each strand in equal molar amounts in a suitable buffer: e.g. 100 mM potassium acetate; 30 mM HEPES, pH 7.5) and heated to 94° C. for 2 minutes and gradually cooled. The oligos are predicted to have a lot of secondary structure, thus a more gradual cooling/annealing step is beneficial. This is done by placing the oligo solution in a water bath or heat block and unplugging/turning off the machine. The annealed oligonucleotides can be diluted in a nuclease free buffer and stored in their double-stranded annealed form at 4° C. The ceDNA plasmid with the TLR-9 inhibitory oligo sequence is then purified (e.g. by gel electrophoresis or column) and is used to make cDNA vector. A ceDNA can the be prepared that encodes Factor IX and that comprises a TLR-9 antagonist.

### Example 8: Controlled Transgene Expression from ceDNA: Transgene Expression from the

ceDNA vector in vivo can be sustained and/or increased by re-dose administration.

A ceDNA vector was produced according to the methods described in Example 1 above, using a ceDNA plasmid comprising a CAG promoter (SEQ ID NO: 3) and a luciferase transgene (SEQ ID NO: 71) is used as an exemplary inflammasome antagonist, flanked between asymmetric ITRs (e.g., a 5′ WT-ITR (SEQ ID NO: 1) and a 3′ mod-ITR (SEQ ID NO: 2) and was assessed in different treatment paragams in vivo. This ceDNA vector was used in all subsequent experiments described in Examples 6-10. In Example 6, the ceDNA vector was purified and formulated with a lipid nanoparticle (LNP ceDNA) and injected into the tail vein of each CD-1® IGS mice. Liposomes were formulated with a suitable lipid blend comprising four components to form lipid nanoparticles (LNP) liposomes, including cationic lipids, helper lipids, cholesterol and PEG-lipids.

To assess the sustained expression of the transgene in vivo from the ceDNA vector over a long time period, the LNP-ceDNA was administered in sterile PBS by tail vein intravenous injection to CD-1® IGS mice of approximately 5-7 weeks of age. Three different dosage groups were assessed: 0.1 mg/kg, 0.5 mg/kg, and 1.0 mg/kg, ten mice per group (except 1.0 mg/kg which had 15 mice per group). Injections were administered on day 0. Five mice from each of the groups were injected with an additional identical dose on day 28. Luciferase expression was measured by IVIS imaging following intravenous administration into CD-i® IGS mice (Charles River Laboratories; WT mice). Luciferase expression was assessed by IVIS imaging following intraperitoneal injection of 150 mg/kg luciferin substrate on days 3, 4, 7, 14, 21, 28, 31, 35, and 42, and routinely (e.g., weekly, biweekly or every 10-days or every 2 weeks), between days 42-110 days. Luciferase transgene expression as the exemplary inflammasome antagonist as measured by IVIS imaging for at least 132 days after 3 different administration protocols (data not shown).

An extension study was performed to investigate the effect of a re-dose, e.g., a re-administration of LNP-ceDNA expressing luciferase of the LNP-ceDNA treated subjects. In particular, it was assessed to determine if expression levels can be increased by one or more additional administrations of the ceDNA vector.

In this study, the biodistribution of luciferase expression from a ceDNA vector was assessed by IVIS in CD-1® IGS mice after an initial intravenous administration of 1.0 mg/kg (i.e., a priming dose) at days 0 and 28 (Group A). A second administration of a ceDNA vector was administered via tail vein injection of 3 mg/kg (Group B) or 10 mg/kg (Group C) in 1.2 mL in the tail vein at day 84. In this study, five (5) CD-1® mice were used in each of Groups A, B and C. IVIS imaging of the mice for luciferase expression was performed prior to the additional dosing at days 49, 56, 63, and 70 as described above, as well as post-redose on day 84 and on days 91, 98, 105, 112, and 132. Luciferase expression was assessed and detected in all three Groups A, B and C until at least 110 days (the longest time period assessed).

The level of expression of luciferase was shown to be increased by a re-dose (i.e., re-administration of the ceDNA composition) of the LNP-ceDNA-Luc, as determined by assessment of luciferase activity in the presence of luciferin. Luciferase transgene expression as an exemplary inflammasome antagonist as measured by IVIS imaging for at least 110 days after 3 different administration protocols (Groups A, B and C). The mice that had not been given any additional redose (1 mg/kg priming dose (i.e., Group A) treatment had stable luciferase expression observed over the duration of the study. The mice in Group B that had been administered a re-dose of 3 mg/kg of the ceDNA vector showed an approximately seven-fold increase in observed radiance relative to the mice in Group C. Surprisingly, the mice re-dosed with 10 mg/kg of the ceDNA vector had a 17-fold increase in observed luciferase radiance over the mice not receiving any redose (Group A).

Group A shows luciferase expression in CD-i® IGS mice after intravenous administration of 1 mg/kg of a ceDNA vector into the tail vein at days 0 and 28. Group B and C show luciferase expression in CD-i® IGS mice administered 1 mg/kg of a ceDNA vector at a first time point (day 0) and re-dosed with administration of a ceDNA vector at a second time point of 84 days. The second administration (i.e., re-dose) of the ceDNA vector increased expression by at least 7-fold, even up to 17-fold.

A 3-fold increase in the dose (i.e., the amount) of ceDNA vector in a re-dose administration in Group B (i.e., 3 mg/kg administered at re-dose) resulted in a 7-fold increase in expression of the luciferase. Also unexpectedly, a 10-fold increase in the amount of ceDNA vector in a re-dose administration (i.e., 10 mg/kg re-dose administered) in Group C resulted in a 17-fold increase in expression of the luciferase. Thus, the second administration (i.e., re-dose) of the ceDNA increased expression by at least 7-fold, even up to 17-fold. This shows that the increase in transgene expression from the re-dose is greater than expected and dependent on the dose or amount of the ceDNA vector in the re-dose administration, and appears to be synergistic to the initial transgene expression from the initial priming administration at day 0. That is, the dose-dependent increase in transgene expression is not additive, rather, the expression level of the transgene is dose-dependent and greater than the sum of the amount of the ceDNA vector administered at each time point.

Both Groups B and C showed significant dose-dependent increase in expression of luciferase as compared to control mice (Group A) that were not re-dosed with a ceDNA vector at the second time point. Taken together, these data show that the expression of a transgene from ceDNA vector can be increased in a dose-dependent manner by re-dose (i.e., re-administration) of the ceDNA vector at least a second time point.

Taken together, these data demonstrate that the expression level of a transgene, e.g., inflammasone antagonist from ceDNA vectors can be maintained at a sustained level for at least 84 days and can be increased in vivo after a redose of the ceDNA vector administered at least at a second time point.

### Example 9: Synthetic Nanocarriers with Super-Saturated Amounts of Rapamycin

Nanocarrier compositions containing the polymers PLGA (3:1 lactide:glycolide, inherent viscosity 0.39 dL/g) and PLA-PEG (5 kDa PEG block, inherent viscosity 0.36 dL/g) as well as the agent rapamycin (RAPA) can be synthesized using an oil-in-water emulsion evaporation method. The organic phase is formed by dissolving the polymers and RAPA in dichloromethane. The emulsion is formed by homogenizing the organic phase in an aqueous phase containing the surfactant polyvinylalcohol (PVA). The emulsion is then combined with a larger amount of aqueous buffer and mixed to allow evaporation of the solvent. The RAPA content in the different compositions is varied such that the compositions crossed the RAPA saturation limit of the system as the RAPA content is increased. The RAPA content at the saturation limit for the composition is calculated using the solubility of the RAPA in the aqueous phase and in the dispersed nanocarrier phase. For compositions containing PVA as the primary solute in the aqueous phase, it is found that the RAPA solubility in the aqueous phase is proportional to the PVA concentration such that the RAPA is soluble at a mass ratio of 1:125 to dissolved PVA. For compositions containing the described PLGA and PLA-PEG as the nanocarrier polymers, it is found that the RAPA solubility in the dispersed nanocarrier phase is 7.2% wt/wt. The following formula can be used to calculate the RAPA content at the saturation limit for the composition:

RAPA content=V(0.008c_{PVA}+0.072cₚₒₗ)

where c_{PVA} is the mass concentration of PVA, cₚₒₗ is the combined mass concentration of the polymers, and V is the volume of the nanocarrier suspension at the end of evaporation.

TABLE 8

| | Calc. Over | RAPA | |
|---|---|---|---|
| | Saturation | Load | Diameter |
| Sample ID | (%) | (%) | (nm) |
| 1 | −50 | 2.5 | 143 |
| 2 | −25 | 3.8 | 146 |
| 3 | 1 | 4.9 | 147 |
| 4 | 23 | 4.9 | 130 |
| 5 | 48 | 8.1 | 160 |
| 6 | 73 | 9.8 | 189 |
| 7 | 98 | 12.4 | 203 |

For 1, 2 and 3, a consistent 60% of the RAPA is not recovered, indicating a sub-saturation equilibrium regime between the aqueous and organic phases. For the remaining nanocarriers containing higher amounts of RAPA, a consistent 6.8 mg of RAPA is not recovered. This consistent absolute mass loss indicates that the system is in an oversaturated regime (i.e., is super-saturated in one or more phases).

### Example 10: Synthetic Nanocarriers with Super-Saturated Rapamycin Eliminates or Delays Antibody Development

Nanocarrier compositions containing the polymers PLGA (3:1 lactide:glycolide, inherent viscosity 0.39 dL/g) and PLA-PEG (5 kDa PEG block, inherent viscosity 0.36 dL/g) as well as the agent RAPA are synthesized using an oil-in-water emulsion evaporation method described in Example 5. The RAPA content in the different compositions is varied such that the compositions crossed the RAPA saturation limit of the system as the RAPA content is increased.

TABLE 9

| | Calc. Over | RAPA | |
|---|---|---|---|
| | Saturation | Load | Diameter |
| Sample ID | (%) | (%) | (nm) |
| 1 | −50 | 2.5 | 143 |
| 3 | 1 | 4.9 | 147 |
| 8 | 21 | 8.5 | 163 |
| 9 | 48 | 13.5 | 159 |

To assess the ability of the compositions to induce immune tolerance, mice are intravenously injected three times weekly with co-administered nanocarrier and keyhole limpet hemocyanin (KLH) and then challenged weekly with KLH only. The sera of the mice are then analyzed for antibodies to KLH after KLH challenge. The compositions made in the super-saturated state, and having final RAPA load of 8% or higher, led to absence or delay of antibody development to KLH to a greater extent than the compositions created at or below saturation and having final RAPA load of 5% or lower.

### Example 11: Synthetic Nanocarriers with Super-Saturated Amounts of Rapamycin

Nanocarrier compositions containing the polymers PLA (inherent viscosity 0.41 dL/g) and PLA-PEG (5 kDa PEG block, inherent viscosity 0.50 dL/g) as well as the agent RAPA were synthesized using the oil-in-water emulsion evaporation method described in Example 9. The RAPA content in the different compositions was varied such that the compositions crossed the RAPA saturation limit of the system as the RAPA content was increased. The RAPA content at the saturation limit for the composition was calculated using the method described in Example 9. For compositions containing the described PLA and PLA-PEG as the nanocarrier polymers, it was found that the RAPA solubility in the dispersed nanocarrier phase is 8.4% wt/wt. The following formula was used to calculate the RAPA content at the saturation limit for the composition:

RAPA content=V(0.008c_{PVA}+0.084cₚₒₗ)

where c_{PVA} is the mass concentration of PVA, cₚₒₗ is the combined mass concentration of the polymers, and V is the volume of the nanocarrier suspension at the end of evaporation. All nanocarrier lots are filtered through 0.22 μm filters at the end of formation.

TABLE 10

| | Calc. Over | RAPA | Unwashed | Final | Filtered |
|---|---|---|---|---|---|
| Sample | Saturation | Load | Diameter | Diameter | Throughput |
| ID | (%) | (%) | (nm) | (nm) | (g/m²) |
| 10 | −10 | 5.4 | 145 | 149 | >171 |
| 11 | 0 | 6.2 | 150 | 155 | >180 |
| 12 | 10 | 6.1 | 151 | 154 | >170 |
| 13 | 20 | 6.1 | 148 | 148 | 80 |
| 14 | 30 | 6.2 | 171 | 151 | 28 |
| 15 | 40 | 5.8 | 202 | 154 | 16 |

Despite adding increasing amount of RAPA to nanocarriers 12-15, the final RAPA content in the nanocarriers did not increase while filter throughput decreased. This indicates that the compositions were oversaturated with RAPA, and the excess RAPA is removed during washing and/or filtration.

### Example 12: Factor IX or VIII for Hemophilia B with ceDNA Encoding Factor IX or Factor VIII Co-Administered with Rapamycin

The experiment is conducted in Factor IX or Factor VIII deficient mice that contain a knock-in of hFIX or hFVIII sequence with a deleterious mutation (e.g. R333Q for hF1X). Male Factor IX or FVIII knockout mice receive single or repeat doses of LNP-ceDNA (Lipid nanoparticle ceDNA) co-administered with rapamycin, or rapamycin analog, wherein the LNP-ceDNA and rapamycin, or rapamycin analog are contained in separate compositions. The LNP-ceDNA vectors are co-administered to respective mice at doses between 0.3 and 5 mg/kg in 1.2 mL volume, and nanocarrier rapamycin (e.g., supersaturated Rapamycin (e.g. SVP-rapamycin) as described in Examples 9-11), or analog thereof administered at e.g., 0.05 mg/kg, 0.1 mg/kg up to 5 mg/kg. Therapeutically effective doses are determined by monitoring efficacy of inhibition of immune response (e.g. upon single and repeat dosing) and measuring the desired amount of transgene expression. Each dose is can be administered via i.v. administration. SVP-Rap may be co-administered, for example at day 0 and day 14.

The expression of Factor IX or Factor VIII in plasma is assessed by ELISA as described in Example 4, at various time points, e.g., at 10, 20, 30, 40, 50, 1000 and 200 days or more, etc. Activated partial thromboplastin time and bleeding time can also be measured as a determination of efficacy and effect of co-administration of rapamycin, or analog on Factor VIII or Factor IX expression. It is expected that the mice which receive ceDNA vector co-administered with rapamycin will exhibit increased and/or sustained expression of Factor IX or Factor VIII for a longer period of time, as compared to the mice that receive only ceDNA vector and not rapamycin, or analog thereof. It is further expected upon re-dose, the mice that receive a re-dose of ceDNA vector and rapamycin, will exhibit less activation of cytokine secretion and increased transgene expression duration and therapeutic efficacy as compared to mice that received a re-dose of ceDNA vector in mice where rapamycin is not administered. The timing of co-administration may be staggered by 0, 1, 2, 3, 4, 5, 6, 7, 8 hours.

### Example 13: Factor IX for Hemophilia B with ceDNA Encoding Factor IX and a cGAS Antagonist

The experiment is conducted in Factor IX deficient mice that contain a knock-in of hFIX sequence with a deleterious mutation (R333Q). Male Factor IX knockout mice receive single or repeat doses of LNP-ceDNA (Lipid nanoparticle ceDNA). Two LNP-ceDNA vectors are used; 1) an LNP-ceDNA encoding both human Factor IX (either native human sequence or Padua FIX variants) and encoding Karposi's sarcoma associated herpes virus protein ORF52; LNP-ceDNA encoding only factor IX and not the cGAS inhibitor as the comparative ceDNA vector. The LNP-ceDNA vectors are administered to respective mice at doses between 0.3 and 5 mg/kg in 1.2 mL volume. Each dose is to be administered via i.v. hydrodynamic administration. The expression of Factor IX in plasma is assessed by ELISA as described herein, at various time points, e.g., at 10, 20, 30, 40, 50, 1000 and 200 days or more, etc. Activated partial thromboplastin time and bleeding time is also measured as a determination of efficacy. It is expected that the mice which receive ceDNA vector expressing both hFIX and ORF52 will exhibit increased and/or sustained expression of factor IX for a longer period of time, as compared to the mice that receive ceDNA vector expressing only Factor IX and not ORF52, or other cGAS inhibitor. It is further expected upon re-dose, the mice that receive a re-dose of ceDNA vector comprising both ORF52 and Factor IX, will exhibit less activation of cytokine secretion and increased transgene expression duration and therapeutic efficacy as compared to mice that received a re-dose of ceDNA vector encoding only Factor IX. The cGAS inhibitor and Factor IX can be delivered on different ceDNA vectors, but preferably they are encoded by the same vector, and accordingly inhibition of cGAS occurs in the same cell that receives the ceDNA vector encoding the transgene, such as Factor IX.

### Example 14: Determining Effects of ceDNA and cGAS Antagonists Co-administration on Innate Immune Responses and Factor IX Expression Duration

In order to examine the effects of co-administration of a ceDNA of interest and an inhibitor of cGAS or cGAS antagonist on immune response (e.g., innate immune response) in vitro, reporter lines can be used for functional assays examining cGAS activation. A cGAS reporter cell line useful for such in vitro assays can be a stably co-transfected cell line that expresses full-length human cGAS and a reporter gene, such as secreted alkaline phosphatase (SEAP) reporter gene, under the transcriptional control of a transcription factor response element, such as an NF-kB binding site, an AP-1 binding site, or a combination thereof. For example, reporter cells are plated in 96-well plates. After a pre-determined time period, such as 16 h, cells are stimulated with various amounts of compositions comprising a ceDNA expressing Factor IX, with or without an inhibitor of cGAS. Activity of the reporter gene, such as SEAP, can be analyzed using any method or assay known to one of skill in the art to compare the level of cGAS activation in the presence of the ceDNA of interest with or without an inhibitor of cGAS. It is expected that in the presence of an inhibitor of cGAS, less activation of the reporter molecule is seen.

In addition, cGAS knock-out reporter lines can be used, such as those derived from human THP-1 monocytes, which is a cell line often used to study DNA sensing pathways as they express all the cytosolic DNA sensors identified so far (with the exception of DAI). Such cGAS knock-out reporter lines can express one or more inducible secreted reporter genes, such as Lucia luciferase and SEAP (secreted embryonic alkaline phosphatase). The reporter gene can be under the control of an ISG54 (interferon-stimulated gene) minimal promoter in conjunction with one or more, such as five, IFN-stimulated response elements. The reporter gene can also be under the control of an IFN-β minimal promoter fused to one or more, such as five, copies of a response element, such as an NF-kB response element. cGAS activity in the presence of inhibitors of cGAS in combination with the ceDNAs described herein can be compared in the knock-out cell line versus the parental cell line.

In order to examine the effects of co-administration of a ceDNA of interest and an inhibitor of cGAS or cGAS antagonist on cGAS and STING activation of immune response (e.g., innate immune response) ex vivo, human monocytes can be isolated by, for example, gradient density centrifugation of peripheral blood and magnetic separation. These monocytes can be examined before and after contact with and/or activation with a ceDNA of interest with or without an inhibitor of cGAS, with suitable controls. After treatment, serum and cell supernatants are used for measuring one or more cytokine pathways as a functional readout of activation of the cGAS/STING pathway, such as interleukin (IL)-1β, IL-6, IL-8, interferon (IFN)-γ, monocyte chemoattractant protein (MCP)-1, and/or tumor necrosis factor (TNF)-α, using any assay or method known to a skilled artisan. In addition, nuclear extracts can be used to verify activation of NF-κB, using any assay or method known to a skilled artisan. It is expected that in the presence of an inhibitor of cGAS, less activation of cytokine pathways and cytokine secretion is observed when administering a ceDNA, leading to increased transgene expression duration and therapeutic efficacy.

In order to examine the effects of co-administration of a ceDNA of interest and an inhibitor of cGAS or cGAS antagonist on cGAS and STING activation of immune response (e.g., innate immune response) in vivo, a mouse model can be used. Serum or lymphocyte samples from the mouse are examined before and after contact with and/or activation with a ceDNA expressing a transgene of interest, such as Factor IX, with or without an inhibitor of cGAS, with suitable controls. After treatment, serum and cell supernatants are used for measuring one or more cytokine pathways as a functional readout of activation of the cGAS/STING pathway, such as interleukin (IL)-1β, IL-6, IL-8, interferon (IFN)-γ, monocyte chemoattractant protein (MCP)-1, and/or tumor necrosis factor (TNF)-α, using any assay or method known to a skilled artisan. In addition, nuclear extracts can be used to verify activation of NF-κB, using any assay or method known to a skilled artisan. It is expected that in the presence of an inhibitor of cGAS, less activation and cytokine secretion is observed when administering a ceDNA, leading to increased transgene expression duration and therapeutic efficacy.

### Example 15: Factor IX for Hemophilia B with ceDNA Encoding Factor IX and a TLR-9 Antagonist

The experiment is conducted in Factor IX deficient mice that contain a knock-in of hFIX sequence with a deleterious mutation (R333Q). Male Factor IX knockout mice receive single or repeat doses of LNP-ceDNA (Lipid nanoparticle ceDNA). Two LNP-ceDNA vectors are used; 1) an LNP-ceDNA encoding both human Factor IX (either native human sequence or Padua FIX variants) and encoding Karposi's sarcoma associated herpes virus protein ORF52; LNP-ceDNA encoding only factor IX and not the cGAS inhibitor as the comparative ceDNA vector. The LNP-ceDNA vectors are administered to respective mice at doses between 0.3 and 5 mg/kg in 1.2 mL volume. Each dose is to be administered via i.v. hydrodynamic administration. The expression of Factor IX in plasma is assessed by ELISA as described in Example 4, at various time points, e.g., at 10, 20, 30, 40, 50, 1000 and 200 days or more, etc. Activated partial thromboplastin time and bleeding time is also measured as a determination of efficacy. It is expected that the mice which receive ceDNA vector comprising the TLR-9 antagonist and expressing hFIX will exhibit increased and/or sustained expression of factor IX for a longer period of time, as compared to the mice that receive ceDNA vector expressing only Factor IX and not an TLR-9 inhibitor. It is further expected upon re-dose, the mice that receive a re-dose of ceDNA vector comprising the TLR-9 inhibitor, e.g. the oligo hairpin sequence, and Factor IX will exhibit less activation of cytokine secretion and increased transgene expression duration and therapeutic efficacy as compared to mice that received a re-dose of ceDNA vector encoding only Factor IX. The TLR-9 inhibitor and Factor IX can be delivered on different ceDNA vectors, in trans, but preferably they are encoded by the same vector, and accordingly inhibition of TLR9 occurs in the same cell that receives the ceDNA vector encoding the transgene, such as Factor IX.

### Example 16: Determining Effects of ceDNA and TLR Antagonists on Innate Immune Responses and Transgene Expression Duration

In order to examine the effects of co-administration of a ceDNA of interest and an inhibitor of TLR9 or TLR9 antagonist on innate immune responses in vitro reporter lines can be used for TLR9-dependent functional assays examining downstream effects of TLR9 activation. A TLR9 reporter cell line can be a stably co-transfected cell line which expresses full-length human Toll-like receptor 9 (TLR9) and a reporter gene, such as secreted alkaline phosphatase (SEAP) reporter gene, under the transcriptional control of a transcription factor response element, such as an NF-kB binding site, an AP-1 binding site, or a combination thereof. For example, reporter cells are plated in 96-well plates. After a pre-determined time period, such as 16 h, cells are stimulated with various amounts of compositions comprising a ceDNA expressing a transgene of interest with or without a TLR9 antagonist. Such an antagonist can be a TLR inhibitory oligonucleotide. Activity of the reporter gene, such as SEAP, can be analyzed using any method or assay known to one of skill in the art to determine the level of TLR9 activation in the presence of the ceDNA of interest with or without a TLR9 antagonist. It is expected that in the presence of an inhibitor of TLR9, less activation of the reporter molecule is seen.

In order to examine the effects of co-administration of a ceDNA of interest and an inhibitor of TLR9 or TLR9 antagonist on TLR9-mediated activation of innate immune responses ex vivo, human monocytes can be isolated by, for example, gradient density centrifugation of peripheral blood and magnetic separation. These monocytes can be examined before and after contact with and/or activation with a ceDNA of interest with or without a TLR9 antagonist, with suitable controls. After treatment, serum and cell supernatants are used for measuring one or more cytokine pathways as a functional readout of TLR9 activation, such as interleukin (IL)-1β, IL-6, IL-8, interferon (IFN)-γ, monocyte chemoattractant protein (MCP)-1, and/or tumor necrosis factor (TNF)-α, using any assay or method known to a skilled artisan. In 0914800addition, nuclear extracts can be used to verify activation of NF-κB, using any assay or method known to a skilled artisan. It is expected that in the presence of an inhibitor of TLR9, less activation of cytokine pathways and cytokine secretion is observed when administering a ceDNA, leading to increased transgene expression duration and therapeutic efficacy.

In order to examine the effects of co-administration of a ceDNA of interest and an inhibitor of TLR9 or TLR9 antagonist on TLR9-mediated activation of innate immune responses in vivo, a mouse model can be used. Serum or lymphocyte samples from the mouse are examined before and after contact with and/or activation with a ceDNA expressing a transgene of interest, such as Factor IX, with or without an inhibitor of TLR9, with suitable controls. After treatment, serum and cell supernatants are used for measuring one or more cytokine pathways as a functional readout of activation of the cGAS/STING pathway, such as interleukin (IL)-1β, IL-6, IL-8, interferon (IFN)-γ, monocyte chemoattractant protein (MCP)-1, and/or tumor necrosis factor (TNF)-α, using any assay or method known to a skilled artisan. In addition, nuclear extracts can be used to verify activation of NF-κB, using any assay or method known to a skilled artisan. It is expected that in the presence of an inhibitor of TLR9, less activation and cytokine secretion is observed when administering a ceDNA, leading to increased transgene expression duration and therapeutic efficacy.

### Example 17: Co-Formulation of ceDNA with RAPA into LNP Vectors

In some embodiments it may be desirable to package rapamycin directly into the ceDNA vector. One nonlimiting example for such direct co-formulation of ceDNA and RAPA follows.

Combinations of ceDNA with rapamycin in lipid nanoparticles (LNP) can be prepared by mixing an alcoholic lipid solution containing rapamycin with a ceDNA aqueous solution using a microfluidic device (e.g., NanoAssemblr™) at a ratio of 1:3 (vol/vol) with total flow rates of 12 ml/min. The total lipid to ceDNA weight ratio can be of approximately 10:1 to 30:1. Briefly, an ionizable lipid (e.g., MC3), a non-cationic-lipid (e.g., distearoylphosphatidylcholine (DSPC)), a component to provide membrane integrity (such as a sterol, e.g., cholesterol) and a conjugated lipid molecule (such as a PEG-lipid, e.g., 1-(monomethoxy-polyethyleneglycol)-2,3-dimyristoylglycerol, with an average PEG molecular weight of 2000 (“PEG-DMG”)), are solubilized in alcohol (e.g., ethanol) at a molar ratio of 50:10:38.5:1.5. Rapamycin is then dissolved in lipid solution to the desired concentration. The ceDNA is diluted to 0.2 mg/mL in 25 mM sodium acetate buffer, pH 4. After the LNP is formed (using, e.g., NanoAssemblr™), the alcohol is removed and the sodium acetate buffer is replaced with PBS by dialysis. Alcohol removal and simultaneous buffer exchange can be accomplished by, for example, dialysis or tangential flow filtration. The obtained lipid nanoparticles are filtered through a 0.2 μm pore sterile filter and stored similarly to the ceDNA LNP vectors described above.

### Example 18: Determining Effects of ceDNA Vector and Rapamycin or Rapamycin Analog Co-Administration on Innate Immune Responses and Factor IX Expression Duration

In order to examine the effects of co-administration of a ceDNA of interest and rapamycin, or analog on innate immune responses in vitro reporter lines can be used for functional assays examining downstream effects of TLR and mTORC1 activation. A TLR9 reporter cell line can be a stably co-transfected cell line which expresses full-length human Toll-like receptor 9 (TLR9) and a reporter gene, such as secreted alkaline phosphatase (SEAP) reporter gene, under the transcriptional control of a transcription factor response element, such as an NF-kB binding site, an AP-1 binding site, or a combination thereof. For example, reporter cells are plated in 96-well plates. After a pre-determined time period, such as 16 h, cells are stimulated with various amounts of compositions comprising a ceDNA expressing a transgene of interest with or without rapamycin or analog thereof. Activity of the reporter gene, such as SEAP, can be analyzed using any method or assay known to one of skill in the art to determine the level of mTORC1 activation in the presence of the ceDNA of interest with or without rapamycin, or analog thereof. It is expected that in the presence of rapamycin, more activation of the reporter molecule is seen, and that STAT3 induction of cytokine IL-10, and other cytokines will be diminished.

In order to examine the effects of co-administration of a ceDNA of interest and rapamycin on activation of innate immune responses ex vivo, human monocytes can be isolated by, for example, gradient density centrifugation of peripheral blood and magnetic separation. These monocytes can be examined before and after contact with and/or activation with a ceDNA of interest with or without rapamycin, or analog thereof, with suitable controls. After treatment, serum and cell supernatants are used for measuring one or more cytokine pathways as a functional readout, such as mTORC1 activation, and/or IL-10 using any assay or method known to a skilled artisan. In addition, nuclear extracts can be used to verify activation of NF-κB, using any assay or method known to a skilled artisan. It is expected that in the presence of rapamycin or analog thereof, less activation of cytokine pathways and cytokine secretion, e.g. IL-10 and Type I IFN is observed when administering a ceDNA, leading to increased transgene expression duration and therapeutic efficacy.

### Example 19: Preparing a ceDNA Vector Co-Expressing Factor IX and a TLR-9 Inhibitor

Oligonucleotides that can form a hairpin structure comprising the following sequences, such as, (TCCTGGCGGGGAAGT, SEQ ID NO: 889), ODN-2114 (TCCTGGAGGGGAAGT, SEQ ID NO: 890), poly-G (GGGGGGGGGGGGGGGGGGGG, SEQ ID NO: 891), ODN-A151 (TTAGGGTTAGGGTTAGGGTTAGGG, SEQ ID NO: 892), G-ODN (CTCC-TATTGGGGGTTTCCTAT, SEQ ID NO: 893), IRS-869 (TCCTGGAGGGGTTGT, SEQ ID NO: 894), INH-1 (CCTGGATGGGAATTCCCATCCAGG, SEQ ID NO: 895), INH-4 (TTCCCATCCAGGCCTGGATGGGAA, SEQ ID NO: 896), (IRS-661 TGCTTGCAAGCTT-GCAAGCA, SEQ ID NO: 897), 4024 (TCCTGGATGGGAAGT, SEQ ID NO: 898), 4084F (CCTGGATGGGAA, SEQ ID NO: 899), INH-13 (CTTACCGCTGCACCTGGATGGGAA, SEQ ID NO: 900), INH-18 (CCTGGATGGGAACTTACCGCTGCA, SEQ ID NO: 901), and IRS-954 TGCTCCTGGAGGGGTTGT, SEQ ID NO: 902) are engineered to have sticky ends after annealing of 5′ to 3′ and complementary 3′ to 5′ strands such that they can be inserted by ligation into a preselected restriction cloning site, e.g. R5 or other site of TTX 9 or TTX 10 plasmid that encodes Factor IX transgene, as described in Example 1 and Example 4.

For example, oligos with appropriate restriction site are annealed by mixing each strand in equal molar amounts in a suitable buffer: e.g. 100 mM potassium acetate; 30 mM HEPES, pH 7.5) and heated to 94° C. for 2 minutes and gradually cooled. The oligos are predicted to have a lot of secondary structure, thus a more gradual cooling/annealing step is beneficial. This is done by placing the oligo solution in a water bath or heat block and unplugging/turning off the machine. The annealed oligonucleotides can be diluted in a nuclease free buffer and stored in their double-stranded annealed form at 4° C. The ceDNA vector with the TLR-9 inhibitory oligo sequence is then purified (e.g. by gel electrophoresis or column) and is used to make cDNA vector. A ceDNA vector can be prepared that encodes Factor IX and that comprises a TLR-9 antagonist as described in Examples 2-3. Methods for determining the effects of co-administration of a ceDNA vector expressing a TLR-9 inhibitor and a rapamycin or a rapamycin analog are described herein.

### Example 20: Preparing a ceDNA Vector Co-Expressing Factor IX and a cGAS Inhibitor

Kaposi's sarcoma-associated herpesvirus protein ORF52 (SEQ ID NO: 882) or a variant thereof that inhibits cGAS, or a truncated cytoplasmic LANA isoform (LANAΔ161 or SEQ ID NO: 884) lacking amino acids 161-1162 of SEQ ID NO: 883) is operably linked to a promoter and inserted into the restriction cloning site R5 of TTX 9 or TTX 10 plasmid that encodes Factor IX transgene, as described in Example 1 and Example 4. A ceDNA vector is thus prepared that encodes both Factor IX and a cGAS inhibitor as described in Examples 2-3. Methods for determining the effects of co-administration of a ceDNA vector expressing a cGAS inhibitor and a rapamycin or a rapamycin analog are herein.

### Example 21: Sustained Transgene Expression In Vivo of LNP-Formulated ceDNA Vectors

The reproducibility of the results in Example 7 with a different lipid nanoparticle was assessed in vivo in mice. Mice were dosed on day 0 with either ceDNA vector comprising a luciferase transgene driven by a CAG promoter that was encapsulated in an LNP different from that used in Example 6 or with that same LNP comprising polyC but lacking ceDNA or a luciferase gene. Specifically, male CD-1® mice of approximately 4 weeks of age were treated with a single injection of 0.5 mg/kg LNP-TTX-luciferase or control LNP-polyC, administered intravenously via lateral tail vein on day 0. At day 14 animals were dosed systemically with luciferin at 150 mg/kg via intraperitoneal injection at 2.5 mL/kg. At approximately 15 minutes after luciferin administration each animal was imaged using an In Vivo Imaging System (“IVIS”).

Significant fluorescence in the liver was observed in all four ceDNA-treated mice, and very little other fluorescence was observed in the animals other than at the injection site, indicating that the LNP mediated liver-specific delivery of the ceDNA construct and that the delivered ceDNA vector was capable of controlled sustained expression of its transgene for at least two weeks after administration.

### Example 22: Sustained Transgene Expression in the Liver In Vivo from ceDNA Vector Administration

In a separate experiment, the localization of LNP-delivered ceDNA within the liver of treated animals was assessed. A ceDNA vector comprising a functional transgene of interest was encapsulated in the same LNP as used in Example 17 and administered to mice in vivo at a dose level of 0.5 mg/kg by intravenous injection. After 6 hours the mice were terminated and liver samples taken, formalin fixed and paraffin-embedded using standard protocols. RNAscope® in situ hybridization assays were performed to visualize the ceDNA vectors within the tissue using a probe specific for the ceDNA transgene and detecting using chromogenic reaction and hematoxylin staining (Advanced Cell Diagnostics). Imaging analysis confirmed that ceDNA was present in the hepatocyte samples taken from the treated mice. One of skill will appreciate that luciferase can be replaced in ceDNA vector for any nucleic acid sequence selected from Table 5.

### Example 23: Sustained Ocular Transgene Expression of ceDNA In Vivo

The sustainability of ceDNA vector transgene expression in tissues other than the liver was assessed to determine tolerability and expression of a ceDNA vector after ocular administration in vivo. While luciferase was used as an exemplary transgene, one of ordinary skill can readily substitute the luciferase transgene with an inflammasone antagonist sequence from any of those listed in Table 5A-5F.

On day 0, male Sprague Dawley rats of approximately 9 weeks of age were injected sub-retinally with 5 μL of either ceDNA vector comprising a luciferase transgene formulated with jetPEI® transfection reagent (Polyplus) or plasmid DNA encoding luciferase formulated with jetPEI®, both at a concentration of 0.25 μg/μL. Four rats were tested in each group Animals were sedated and injected sub-retinally in the right eye with the test article using a 33 gauge needle. The left eye of each animal was untreated. Immediately after injection eyes were checked with optical coherence tomography or fundus imaging in order to confirm the presence of a subretinal bleb. Rats were treated with buprenorphine and topical antibiotic ointment according to standard procedures.

At days 7, 14, 21, 28, and 35, the animals in both groups were dosed systemically with freshly made luciferin at 150 mg/kg via intraperitoneal injection at 2.5 mL/kg. at 5-15 minutes post luciferin administration, all animals were imaged using IVIS while under isoflurane anesthesia. Total Flux [p/s] and average Flux (p/s/sr/cm²) in a region of interest encompassing the eye were obtained over 5 minutes of exposure. The results were graphed as average radiance of each treatment group in the treated eye (“injected”) relative to the average radiance of each treatment group in the untreated eye (“uninjected”). Significant fluorescence was readily detectable in the ceDNA vector-treated eyes but much weaker in the plasmid-treated eyes. After 35 days, the plasmid-injected rats were terminated, while the study continued for the ceDNA-treated rats, with luciferin injection and IVIS imaging at days 42, 49, 56, 63, 70, and 99. The results demonstrate that ceDNA vector introduced in a single injection to rat eye mediated transgene expression in vivo and that that expression was sustained at a high level at least through 99 days after injection.

### Example 24: Hydrodynamic Delivery of ceDNA

A well-known method of introducing nucleic acid to the liver in rodents is by hydrodynamic tail vein injection. In this system, the pressurized injection in a large volume of non-encapsulated nucleic acid results in a transient increase in cell permeability and delivery directly into tissues and cells. This provides an experimental mechanism to bypass many of the host immune systems, such as macrophage delivery. Accordingly, luciferase expression observed after hydrodynamic injection of naked ceDNA vector was compared to that observed after more traditional intravenous injection of LNP-encapsulated ceDNA. For this experiment, the ceDNA vectors utilized a wild-type AAV2 left ITR and a mutated right ITR.

Briefly, ceDNA vector encoding luciferase under the control of the CAG promoter was prepared and either encapsulated in LNP or left unencapsulated. Adult male CD-1 mice were administered by tail vein injection either (i) the LNP-encapsulated ceDNA vector at a dose of 0.5 mg/kg in a total volume of 5 mL/kg, or (ii) the same vector but unencapsulated, at a dose of 0.01 mg/kg in a total volume of 1.2 mL. There were three mice in each treatment group. Body weights were recorded on days 1, 2, and 3. In-life imaging was performed on days 1 and 3 using an in vivo imaging system (IVIS). For the imaging, each mouse was injected with luciferin at 150 mg/kg via intraperitoneal injection at 2.5 mL/kg. After 15 minutes, each mouse was anaesthetized and imaged.

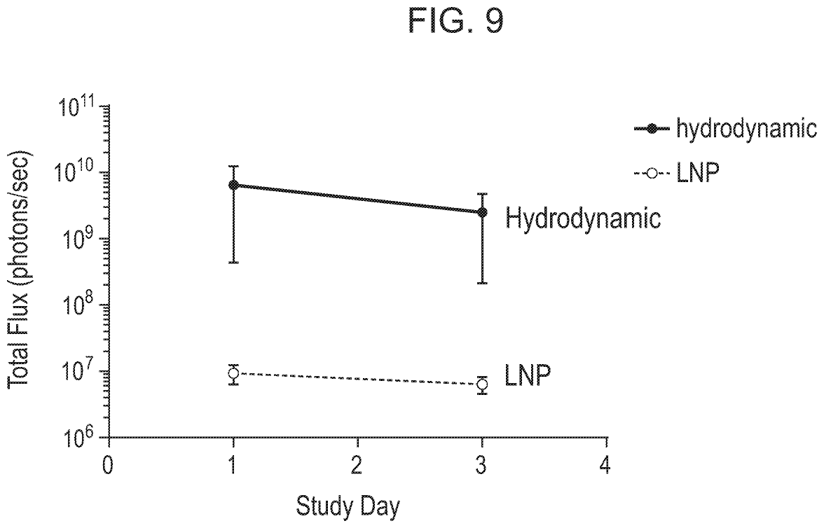

Even though administered at a 50-fold lower dose, the luciferase expression observed in the hydrodynamically injected mice was far greater (˜10⁷ maximum total flux) than the non-hydrodynamically injected mice (˜10⁷ maximum total flux) (FIG. 9). It was found in prior studies that administration of the LNP alone without ceDNA vector cargo did not trigger an immune response (data not shown), and thus the differential between the two dose groups may be attributable to engagement of the LNP-encapsulated ceDNA vector of one or more host immune systems and avoidance of those system(s) by hydrodynamic administration.

### Example 25: Modulation of Immune Pathways in Cultured Cells and Impact on ceDNA Vector Expression

A cell-based assay was established to facilitate interrogation of the contribution of various immune pathways to host response to ceDNA administration. The assay uses THP-1 cells (an acute monocytic leukemia cell line) in several variations: THP-1 Dual™ cells (Invitrogen), with stable integration of reporter constructs for detection of both NF-κB activation (TLR9 pathway, via SEAP detection with Quanti-Blue™) and the IRF pathway activation (via a secreted luciferase with Quanti-Luc™), THP-1 cells with a constitutive knockout in the cGAS immune pathway, and THP-1 cells with a constitutive knockout in the STING immune pathway. Using known inhibitors of certain pathways, it is possible to better understand the relative contributions of endogenous immune pathways to an observed immune response to a given stimulus.

Briefly, THP-1 cells in culture were diluted to 0.5×10⁶/mL in Opti-MEM™ media (ThermoFisher), and 150 μL were added to each well of a 96 well plate. The cells were pretreated with inhibitors: the desired inhibitors were diluted into Opti-MEM™ and added to the designated sample wells. For this experiment, A151 (oligonucleotide TTAGGGTTAGGGTTAGGGTTAGGG (SEQ ID NO:892) and BX795 (N-[3-[[5-Iodo-4-[[3-[(2-thienylcarbonyl)amino]propyl]amino]-2-pyrimidinyl]amino]phenyl]-1-pyrrolidinecarboxamide, CAS 702675-74-9) were used at final concentrations in each sample well of 0 μM, 0.625 μM, 1.25 μM, or 2.5 μM. The plates were incubated at 37° C. for 2 hours. 200 ng of the desired ceDNA vector was diluted 1:3 in Lipofectamine™ 3000 and incubated for 5-10 min at room temperature. The ceDNA vector-Lipofectamine complex was then added to sample wells. The plates were incubated for 24 hours at 37° C. The amount of NF-κB activation and IRF2 activation was quantified by the Quanti-Blue™ and Quanti-Luc™ kits, respectively, according to the manufacturer's instructions.

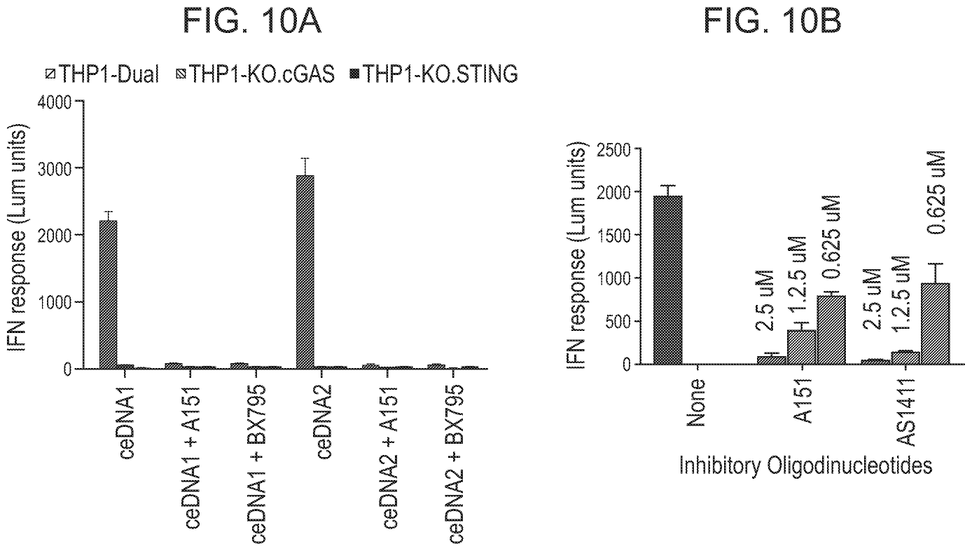

Administration of two different preparations of ceDNA vector to the THP-1 dual reporter cells both resulted in significant induction of interferon, indicating activation of at least one immune pathway FIG. 10A). Notably, no induction of interferon was observed when either of the two THP-1 knockout strains were treated with ceDNA at the same concentration (FIG. 10A), indicating that the cGAS/STING pathway is involved in cytokine induction in response to ceDNA administration. A similar result was found when the THP-1 dual reporter cells were treated with both ceDNA and BX795; BX795 is a STING pathway-specific inhibitor and its abrogation of ceDNA-induced interferon induction suggests again that the STING pathway is involved (FIG. 10A). A151 is known to inhibit the cGAS/STING pathway, the TLR9 pathway, and also inflammasome-mediated immune pathways. It had a similar effect to that observed with BX795 treatment (FIG. 10A).

A second experiment assayed the concentrations of inhibitor needed to observe a protective effect upon ceDNA administration (FIG. 10B). For both A151 and AS1411, the observed inhibition of interferon induction was concentration-dependent, with maximal inhibition observed at a concentration of 2.5 μM (FIG. 10B).

### Example 26: Impact of Modulation of ceDNA Unmethylated CpG Content on Immune Response

CpG motifs in a gene sequence are known to stimulate the TLR9 DNA sensing pathway. Accordingly, the impact of reduction of CpG motifs in a ceDNA construct sequence on innate immune pathway activation upon introduction of that sequence in vivo was investigated.

A. Cell-Based Assays Testing the Impact of Minimization of ceDNA Unmethylated CpG

Studies were performed to assess (i) TLR9 pathway activation in response to ceDNA administration and (ii) the effect of modulation of CpG presence/methylation status on such activation. For this particular study, a ceDNA vector was used that expressed a green fluorescent protein and comprised a wild-type left ITR and a mutant right ITR.

HEK-293 cells expressing human TLR9 (HEK-BLUE.hTLR9 cells, InvivoGen) were seeded in a 96 well plate at 50,000 cells per well. The plates were incubated overnight at 37° C. For ceDNA samples undergoing methylation pretreatment, ceDNA vector, buffer, S-adenosyl methionine, CpG methyltransferase, and water to a total reaction volume of 50 μL following art-known methods. The reaction was incubated at 37° C. for 1 hour, then stopped by heating to 65° C. for 20 min. The ceDNA was purified from the reaction mixture using a commercially available purification kit (PCR clean kit, Qiagen®), and the resulting DNA concentration was measured.

The cells were pretreated for 3 hours with any desired inhibitors—in this experiment, A151 was used at a final concentration per well of 10 μM. After the pretreatment, cells were transfected with 300 ng ceDNA in a 1:3 ratio with Lipofectamine 3000, diluted in Opti-MEM™, or a positive control ODN2006, known to stimulate the TLR9 pathway. The cells were incubated for 24 hours at 37° C. and 5% CO₂. Seap expression (a component of the TLR9 pathway) was then measured using Quanti-BLUE™ (InvivoGen).

As shown in FIG. 11A, ODN2006 induces a robust NF-κB response; the ceDNA construct induced a lesser response, and when pre-methylated, the response dropped to background levels. When combined with A151 (known to inhibit the TLR9 pathway), the ceDNA-treated samples also displayed minimal levels of NF-κB induction (FIG. 11B). This demonstrates first, that the TLR9 pathway contributes to the host immune response to ceDNA administration. Further, minimization of CpG content by methylation eliminated the majority of the TLR9 activation by ceDNA, and this effect could be mimicked by pretreatment of the cells with A151 without altering the CpG content or methylation status.

B. Murine Studies Assessing the Impact of ceDNA Unmethylated CpG Minimization

The impact of CpG minimization in ceDNA vectors was also assessed in mice.

Cytokine response and ceDNA-encoded gene expression upon administration of ceDNA vectors to mice was measured.

Three different ceDNA vectors were used, each encoding luciferase as the transgene. The first ceDNA vector had a high number of unmethylated CpG (˜350) (“ceDNA High CpG”) and comprised the constitutive CAG promoter; the second had a moderate number of unmethylated CpG (˜60) (“ceDNA Low CpG”) and comprised the liver-specific hAAT promoter; and the third was a methylated form of the second, such that it contained no unmethylated CpG (“ceDNA No CpG”), also comprising the hAAT promoter. The ceDNA vectors were otherwise identical. The vectors were prepared as described above.

Four groups of four male CD-1 mice, approximately 4 weeks old, were treated with one of the ceDNA vectors encapsulated in an LNP or a polyC control. On day 0 each mouse was administered a single intravenous tail vein injection of 0.5 mg/kg ceDNA vector in a volume of 5 mL/kg. Body weights were recorded on days −1, −, 1, 2, 3, 7, and weekly thereafter until the mice were terminated. Whole blood and serum samples were taken on days 0, 1, and 35. In-life imaging was performed on days 7, 14, 21, 28, and 35, and weekly thereafter using an in vivo imaging system (IVIS). For the imaging, each mouse was injected with luciferin at 150 mg/kg via intraperitoneal injection at 2.5 mL/kg. After 15 minutes, each mouse was anaesthetized and imaged. The mice were terminated at day 93 and terminal tissues collected, including liver and spleen. Cytokine measurements were taken 6 hours after dosing on day 0.

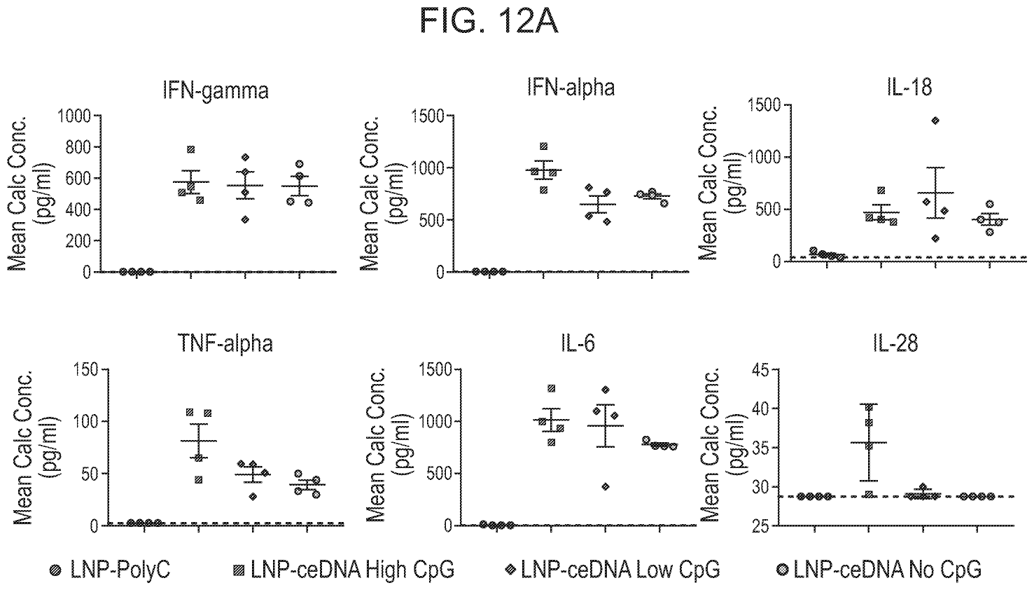

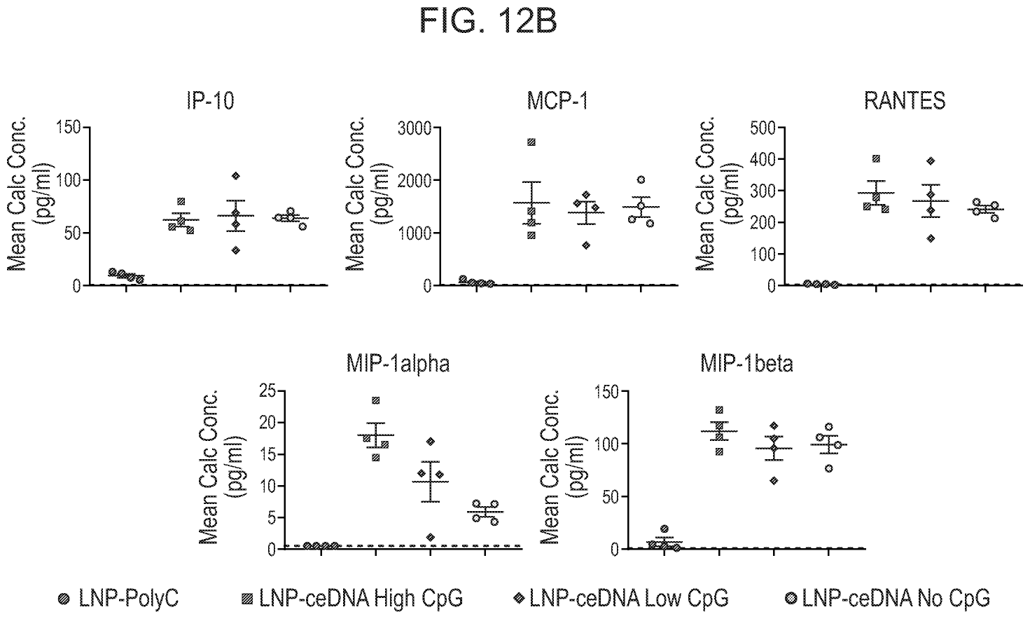

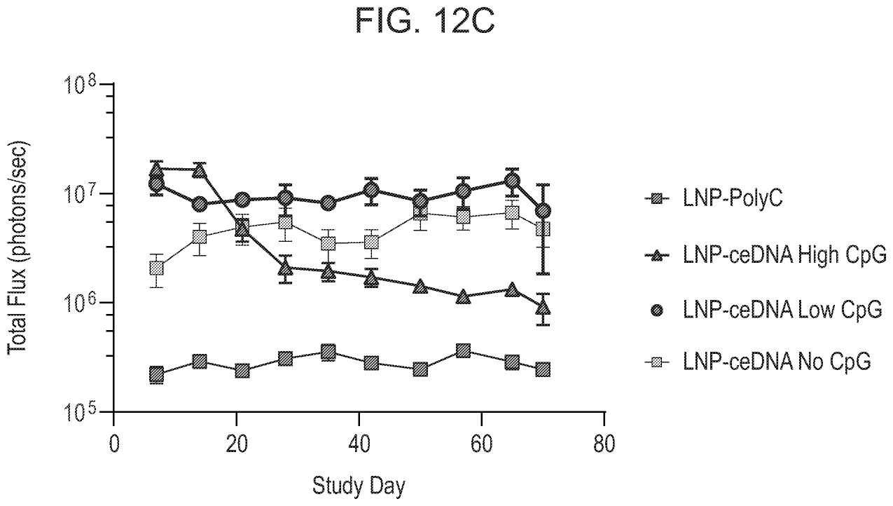

Similar body weight loss was observed in each of the ceDNA-treated mouse groups (5-7%), followed by rapid recovery by day 7. Cytokine analyses from the day 0 samples showed that while many of the assessed cytokines were similarly elevated across all treatment groups, interferon alpha, tumor necrosis factor alpha, and MIP-1 alpha were all reduced in the Low- or No-CpG samples relative to the High CpG samples (FIG. 12A and FIG. 12B).

While both the Low CpG and High CpG ceDNA-treated mice displayed significant fluorescence at days 7 and 14, the fluorescence decreased rapidly in the High CpG mice after day 14 and steadily decreased for the remainder of the study. In contrast, the total flux for the Low CpG and No CpG ceDNA-treated mice remained at a steady high level (FIG. 12C), suggesting that keeping the unmethylated CpG presence in the ceDNA vector under some threshold, and thereby not triggering the TLR9 pathway, helps avoid the otherwise observed more rapid decline in ceDNA-encoded protein expression (and hence, fluorescence) in this study.

### Example 27: Expression and Host Response in Neonatal Mice

The prior experiments showed that the cGAS/STING pathway is at least partly implicated in the cytokine induction response observed upon ceDNA vector administration to cells. This pathway is known to become active later in development, such that neonatal mice with immature immune systems lack an active cGAS/STING pathway. Accordingly, a neonatal mouse experiment was undertaken to examine the effect of the pathway's absence on ceDNA vector expression and persistence.

A ceDNA vector encoding luciferase as the transgene, with a wild-type AAV2 left ITR and a mutant right ITR and a CAG promoter was used. The ceDNA vector was prepared as described above. ceDNA vector samples or a poly C control were intravenously administered via tail vein injection to neonatal (8 day old) male CD-1 mice at a dose level of 0.1 or 0.5 mg/kg in a volume of up to 5 mL/kg. Five replicates were included in each sample group. Body weights were recorded on day one and the three days following. In-life imaging was performed on days 7, 14, and 21 using an in vivo imaging system (IVIS). For the imaging, each mouse was injected with luciferin at 150 mg/kg via intraperitoneal injection at 2.5 mL/kg. After 15 minutes, each mouse was anaesthetized and imaged.

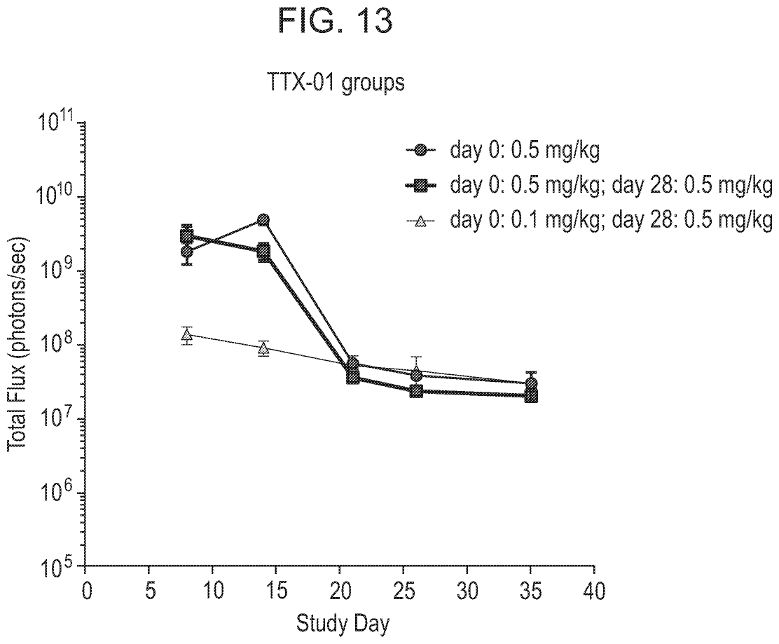

Notably, no body weight loss was observed in any of the treatment groups after the day zero injection. High levels of total flux (representative of luciferase expression from the introduced ceDNA vectors) were observed in all ceDNA-administered animals, with the 0.5 mg/kg dose resulting in an expression level approximately 1 log higher than the 0.1 mg/kg dose over the first 14 days (FIG. 13). Thereafter, the expression level stabilized and persisted at the same level in both dose groups. Compared to similar studies in adult CD-1 mice, the ceDNA vector expression level in the neonatal mice even after 14 days was at least two log greater (data not shown). This result suggests that avoidance of cGAS/STING pathway activation is beneficial in fostering ceDNA vector expression and persistence.

### Example 28: Impact of Modulation of Multiple Immune Pathways on ceDNA Persistence, Expression, and Cytokine Induction

The prior studies assessed the effects of TLR9 pathway modulation in both cultured cell and murine systems. However, multiple molecular pathways are known to be involved in host response to foreign DNA, and the impact of avoidance of triggering the TLR9 pathway may not be readily observed if one or more other pathways continue to be engaged by ceDNA administration. To test this, CpG minimized ceDNA vectors were tested in the context of a goldenticket mouse strain, which has a mutation abrogating STING function. Thus, the experiment permitted interrogation of the TLR9 pathway without confounding cGAS/STING pathway activity.

Three different ceDNA vectors were used, each encoding luciferase as the transgene. The first ceDNA vector had a high number of unmethylated CpG (˜350) (“ceDNA High CpG”) and comprised a constitutive promoter (cET), the second had a moderate number of unmethylated CpG (˜60) (“ceDNA Low CpG”) and the third had a small number of CpG (˜36) but was methylated such that it contained no unmethylated CpG (“ceDNA No CpG”). Both the second and third constructs comprised the liver-specific hAAT promoters. The ceDNA vectors were otherwise identical. The vectors were prepared as described above.

Each of the ceDNA vector samples or a poly C control were intravenously administered via tail vein injection to adult male goldenticket mice (Tmem173^{gt}) at a dose level of 0.5 mg/kg in a volume of 5 mL/kg. In some cases, a second dose of the ceDNA vector sample was administered to the mice at day 22. Four replicates were included in each sample group. Body weights were recorded on dose days and the three days following. Whole blood and serum samples were taken on days 0 (6 hours post dose) and day 22 (6 hours post dose). In-life imaging was performed on days 7, 14, 22, 29, 36 and 43 using an in vivo imaging system (IVIS). For the imaging, each mouse was injected with luciferin at 150 mg/kg via intraperitoneal injection at 2.5 mL/kg. After 15 minutes, each mouse was anaesthetized and imaged. The mice were terminated at day 43 and terminal tissues collected, including liver and spleen. Cytokine measurements were taken from blood draws on Day 0 and 22.

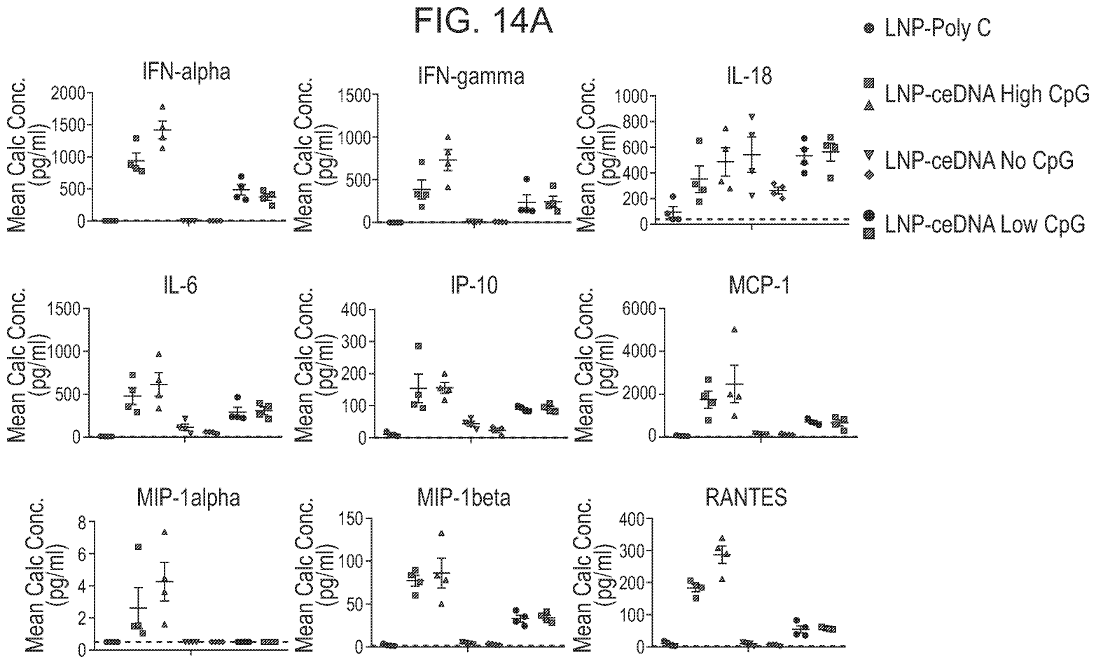

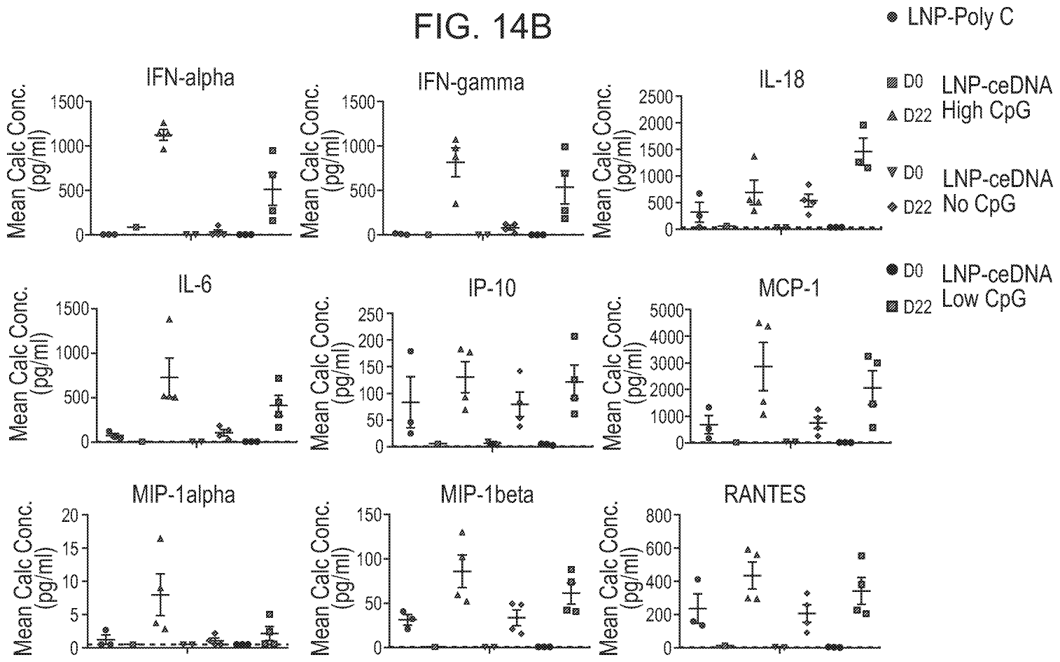

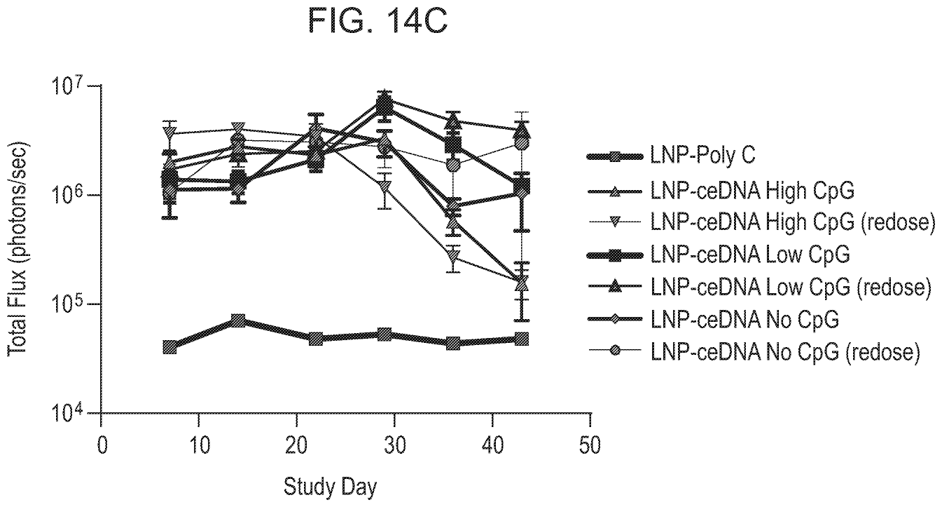

Body weight loss upon ceDNA administration was less than 5% and was essentially recovered in all cases by day 3. Upon readministration at day 22, the treated mice again lost <5% of body weight and regained it rapidly in the following days. Cytokine induction was assessed from the day 0 blood samples (FIG. 14A). With the exception of IL-18, the levels of each of the assayed cytokines correlated with the degree of presence of CpG in the ceDNA construct (FIG. 14A), with low to no induction observed in the ceDNA No CpG treated mice of IFN-alpha, IFN-gamma, IL-6, IP-10, MCP-1, MIP-1alpha, MIP-1 beta and RANTES. In the re-dosed mice, all of the samples showed increases in all cytokine levels relative to the day 0 reads (FIG. 14B), but again in all cases except IL-18, the degree of activation correlated with the amount of CpG present in the administered ceDNA vector.

Expression of luciferase in the different treatment groups was similar through day 22 (FIG. 14C). After that point both single-dose and the re-dosed ceDNA High CpG samples had sharp declines in total flux, while the Low CpG and No CpG groups either maintained consistent total flux measurements or were attenuated in signal loss relative to the High CpG group. The combined results demonstrate that minimization of CpG content in the administered ceDNA vectors—and by extension, avoidance of engaging the TLR9 innate immune pathway—contributed to marked drops in cytokine induction and more robust persistence of gene expression from the ceDNA in treated goldenticket mice.

### Example 29: Sustained Dosing and Redosing of ceDNA Vector in Rag2 Mice

In situations where one or more of the transgenes encoded in the gene expression cassette of the ceDNA vector is expressed in a host environment (e.g., cell or subject) where the expressed protein is recognized as foreign, the possibility exists that the host will mount an adaptive immune response that may result in undesired depletion of the expression product, which could potentially be confused for lack of expression. In some cases this may occur with a reporter molecule that is heterologous to the normal host environment. Accordingly, ceDNA vector transgene expression was assessed in vivo in the Rag2 mouse model which lacks B and T cells and therefore does not mount an adaptive immune response to non-native murine proteins such as luciferase. Briefly, c57bl/6 and Rag2 knockout mice were dosed intravenously via tail vein injection with 0.5 mg/kg of LNP-encapsulated ceDNA vector expressing luciferase or a polyC control at day 0, and at day 21 certain mice were redosed with the same LNP-encapsulated ceDNA vector at the same dose level. All testing groups consisted of 4 mice each. IVIS imaging was performed after luciferin injection at weekly intervals.

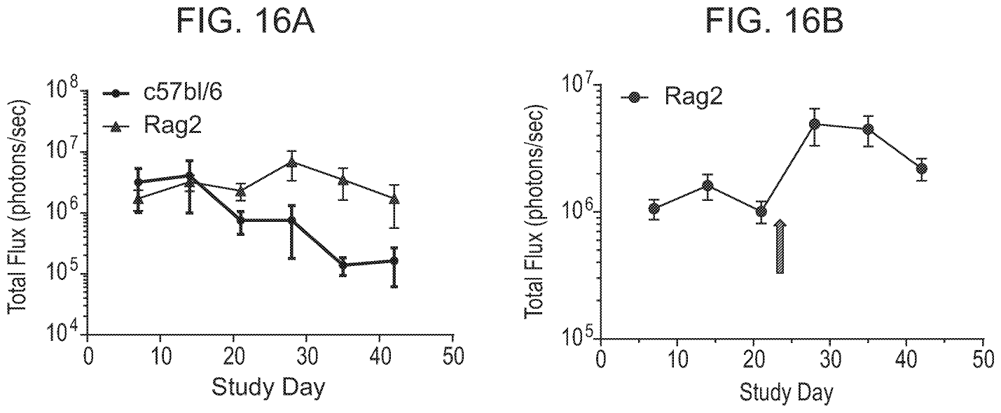

Comparing the total flux observed from the IVIS analyses, the fluorescence observed in the wild-type mice (an indirect measure of the presence of expressed luciferase) dosed with LNP-ceDNA vector-Luc decreased gradually after day 21 whereas the Rag2 mice administered the same treatment displayed relatively constant sustained expression of luciferase over the 42 day experiment (FIG. 16A). The approximately 21-day time point of the observed decrease in the wild-type mice corresponds to the timeframe in which an adapative immune response might expect to be produced. Re-administration of the LNP-ceDNA vector in the Rag2 mice resulted in a marked increase in expression which was sustained over the at least 21 days it was tracked in this study (FIG. 16B). The results suggest that adaptive immunity may play a role when a non-native protein is expressed from a ceDNA vector in a host, and that observed decreases in expression in the 20+ day timeframe from initial administration may signal a confounding adaptive immune response to the expressed molecule rather than (or in addition to) a decline in expression. Of note, this response is expected to be low when expressing native proteins in a host where it is anticipated that the host will properly recognize the expressed molecules as self and will not develop such an immune response.

### Example 30: Impact of Liver-Specific Expression and CpG Modulation on Sustained Expression

As described in Example 29, undesired host immune response may in some cases artificially dampen what would otherwise be sustained expression of one or more desired transgenes from an introduced ceDNA vector. Two approaches were taken to assess the impact of avoiding and/or dampening potential host immune response on sustained expression from a ceDNA vector. First, since the ceDNA-Luc vector used in the preceding examples was under the control of a constitutive CAG promoter, a similar construct was made using a liver-specific promoter (hAAT) or a different constitutive promoter (hEF-1) to see whether avoiding prolonged exposure to myeloid cells or non-liver tissue reduced any observed immune effects. Second, certain of the ceDNA-luciferase constructs were engineered to be reduced in CpG content, a known trigger for host immune reaction. ceDNA-encoded luciferase gene expression upon administration of such engineered and promoter-switched ceDNA vectors to mice was measured.

Three different ceDNA vectors were used, each encoding luciferase as the transgene. The first ceDNA vector had a high number of unmethylated CpG (˜350) and comprised the constitutive CAG promoter (“ceDNA CAG”); the second had a moderate number of unmethylated CpG (˜60) and comprised the liver-specific hAAT promoter (“ceDNA hAAT low CpG”); and the third was a methylated form of the second, such that it contained no unmethylated CpG and also comprised the hAAT promoter (“ceDNA hAAT No CpG”). The ceDNA vectors were otherwise identical. The vectors were prepared as described above.

Four groups of four male CD-1® mice, approximately 4 weeks old, were treated with one of the ceDNA vectors encapsulated in an LNP or a polyC control. On day 0 each mouse was administered a single intravenous tail vein injection of 0.5 mg/kg ceDNA vector in a volume of 5 mL/kg. Body weights were recorded on days −1, −, 1, 2, 3, 7, and weekly thereafter until the mice were terminated. Whole blood and serum samples were taken on days 0, 1, and 35. In-life imaging was performed on days 7, 14, 21, 28, and 35, and weekly thereafter using an in vivo imaging system (IVIS). For the imaging, each mouse was injected with luciferin at 150 mg/kg via intraperitoneal injection at 2.5 mL/kg. After 15 minutes, each mouse was anaesthetized and imaged. The mice were terminated at day 93 and terminal tissues collected, including liver and spleen. Cytokine measurements were taken 6 hours after dosing on day 0.

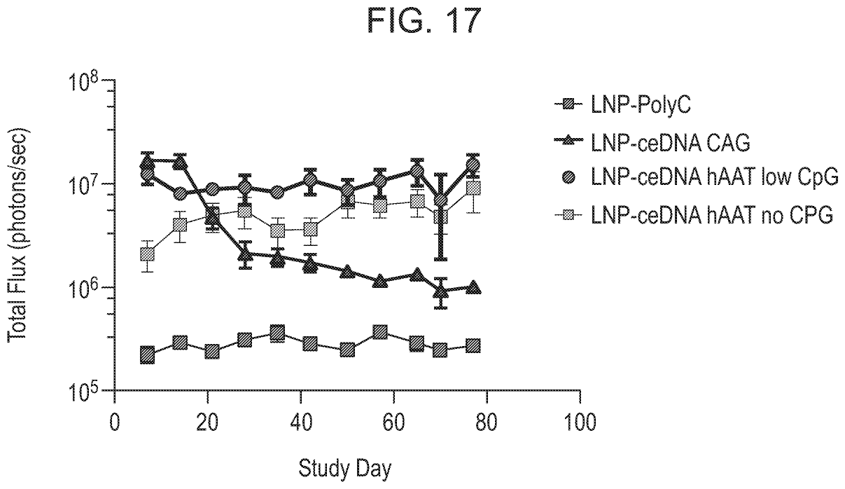

While all of the ceDNA-treated mice displayed significant fluorescence at days 7 and 14, the fluorescence decreased rapidly in the ceDNA CAG mice after day 14 and more gradually decreased for the remainder of the study. In contrast, the total flux for the ceDNA hAAT low CpG and No CpG-treated mice remained at a steady high level (FIG. 17). This suggested that directing the ceDNA vector delivery specifically to the liver resulted in sustained, durable transgene expression from the vector over at least 77 days after a single injection. Constructs that were CpG minimized or completely absent of CpG content had similar durable sustained expression profiles, while the high CpG constitutive promoter construct exhibited a decline in expression over time, suggesting that host immune activation by the ceDNA vector introduction may play a role in any decreased expression observed from such vector in a subject. These results provide alternative methods of tailoring the duration of the response to the desired level by selecting a tissue-restricted promoter and/or altering the CpG content of the ceDNA vector in the event that a host immune response is observed—a potentially transgene-specific response.

### Example 31: In Vivo Expression of an Inflammasone Antagonist

Upon confirmation of appropriate protein expression and function in recipient cells in vitro, ceDNA vector with sequences encoding an inflammasone antagonist are be formulated with lipid nanoparticles and administered to mice deficient in functional expression of the respective protein production at various time points (in utero, newborn, 4 weeks, and 8 weeks of age), for verification of expression and protein function in vivo.

The LNP-ceDNA vectors are administered to respective mice at doses between 0.3 and 5 mg/kg in 1.2 mL volume. Each dose is to be administered via i.v. hydrodynamic administration or will be administered for example by intraperitoneal injection. Administration to normal mice serves as a control and also can be used to detect the presence and quantity of the therapeutic protein.

Following an acute dosing, e.g. a., single dose of LNP-ceDNA, expression in liver tissue in the recipient mouse will be determined at various time points e.g., at 10, 20, 30, 40, 50, 1000 and 200 days or more, etc. Specifically, samples of the mouse livers and bile duct will be obtained an analyzed for protein presence using immunostaining of tissue sections. Protein presence will be assessed quantitatively and also for appropriate localization within the tissue and cells therein. Cells in the liver (e.g., hepatic and epithelial) and of the bile duct (e.g., cholangiocytes) will be assessed for protein expression.

### Example 32: Preparing a ceDNA Co-Expressing a Therapeutic Gene (e.g., Factor IX) and an Inhibitor of the NLRP3 Inflammasome Pathway

A151 (SEQ ID NO: 892) or a variant thereof that inhibits AIM2 is operably linked to a promoter and inserted into the restriction cloning site R5 of a ceDNA vector as described in Example 1. A ceDNA is thus prepared that encodes both Factor IX and an AIM2 inhibitor.

### Example 33: Confirming Expression of a NLRP3 inflammasome Inhibitor Expressed by a ceDNA

Expression of a desired NLRP3 or AIM2 or caspase-1 inhibitor co-expressed by a ceDNA, such as A151 (SEQ ID NO: 892), can be confirmed using HeLa cells and antibodies specific for the inhibitor. For example, HeLa cells are cultured and transient transfections of the constructs co-expressing the Factor IX and the desired NLRP3 or AIM2 or caspase-1 inhibitor are performed using, for example, Fusegene6 transfection reagent (3:1; fusgene6: DNA). Western blot techniques and/or flow cytometry, as known to those of skill in the art, are used to detect expression of the NLRP3 or AIM2 or caspase-1 inhibitor.

### Example 34: Factor IX for Hemophilia B with ceDNA Encoding Factor IX and an Inhibitor of the NLRP3 Inflammasome Pathway

The experiment is conducted in Factor IX deficient mice that contain a knock-in of hFIX sequence with a deleterious mutation (R333Q). Male Factor IX knockout mice receive single or repeat doses of LNP-ceDNA (Lipid Nanoparticle ceDNA). Two LNP-ceDNA vectors are used; 1) an LNP-ceDNA encoding both human Factor IX (either native human sequence or Padua FIX variants) and encoding A151 (SEQ ID NO: 892); LNP-ceDNA encoding only factor IX and not the cGAS inhibitor as the comparative ceDNA vector. The LNP-ceDNA vectors are administered to respective mice at doses between 0.3 and 5 mg/kg in 1.2 mL volume. Each dose is to be administered via i.v. hydrodynamic administration. The expression of Factor IX in plasma is assessed by ELISA, at various time points, e.g., at 7, 14 and 21 days or more, etc. Activated partial thromboplastin time and bleeding time is also measured as a determination of efficacy. It is expected that the mice which receive ceDNA vector expressing both hFIX and A151 will exhibit increased and/or sustained expression of factor IX for a longer period of time, as compared to the mice that receive ceDNA vector expressing only Factor IX and not A151, or other NLRP3 or AIM2 or caspase-1 inhibitor. It is further expected upon re-dose, the mice that receive a re-dose of ceDNA vector comprising both A151 and Factor IX, will exhibit less activation of cytokine secretion and increased transgene expression duration and therapeutic efficacy as compared to mice that received a re-dose of ceDNA vector encoding only Factor IX. An inhibitor of the NLRP3 inflammasome pathway and Factor IX can be delivered on different ceDNA vectors, but preferably they are encoded by the same vector, and accordingly inhibition of an inhibitor of the NLRP3 inflammasome pathway occurs in the same cell that receives the ceDNA vector encoding the transgene, such as Factor IX.

### Example 35: Determining Effects of ceDNA and NLRP3 Inflammasome Inhibitor Co-Administration on Innate Immune Responses and Factor IX Expression Duration

In order to examine the effects of co-administration of a ceDNA of interest and an inhibitor of the NLRP3 inflammasome pathway on innate immune responses in vitro, reporter lines can be used for functional assays examining NLRP3 inflammasome or caspase-1 activation. A NLRP3 inflammasome reporter cell line useful for such in vitro assays can be a stably co-transfected cell line that expresses full-length NLRP3 and a reporter gene, such as secreted alkaline phosphatase (SEAP) reporter gene, under the transcriptional control of a transcription factor response element, such as an NF-kB binding site, an AP-1 binding site, or a combination thereof. For example, reporter cells are plated in 96-well plates. After a pre-determined time period, such as 16 h, cells are stimulated with various amounts of compositions comprising a ceDNA expressing Factor IX, with or without an inhibitor of the NLRP3 inflammasome. Activity of the reporter gene, such as SEAP, can be analyzed using any method or assay known to one of skill in the art to compare the level of caspase-1 activation, or NLRP3 inflammasome activation in the presence of the ceDNA of interest with or without an inhibitor the NLRP3 inflammasome pathway. It is expected that in the presence of an inhibitor of NLRP3 inflammasome, less activation of the reporter molecule is seen. The same reporter assay can be used to assess inhibitors of caspase-1.

Similarly, in order to examine the effects of co-administration of a ceDNA of interest and an inhibitor of the AIM2 inflammasome pathway on innate immune responses in vitro, reporter lines can be used for functional assays examining AIM2 inflammasome or caspase-1 activation. An AIM2 inflammasome reporter cell line useful for such in vitro assays can be a stably co-transfected cell line that expresses full-length human AIM2 and a reporter gene, such as secreted alkaline phosphatase (SEAP) reporter gene, under the transcriptional control of a transcription factor response element, such as an NF-kB binding site, an AP-1 binding site, or a combination thereof. The assay can be carried out as for the NLRP3 inflammasome reporter assay, where reporter cells, e.g., plated in 96-well plates, after pre-determined period of time, are stimulated with various amounts of compositions comprising a ceDNA expressing Factor IX, with or without an inhibitor of the AIM2 inflammasome. Activity of the reporter gene, such as SEAP, can be analyzed using any method or assay known to one of skill in the art to compare the level of caspase-1 activation, or AIM2 inflammasome activation in the presence of the ceDNA of interest with or without an inhibitor the AIM2 inflammasome pathway. It is expected that in the presence of an inhibitor of AIM2 inflammasome, less activation of the reporter molecule is seen. The same reporter assay can be used to assess inhibitors of caspase-1.

In addition, NLRP3 inflammasome or AIM2 inflammasome knock-out reporter lines can be used, such as THP1-defNLRP3 cells (InvivoGen) or TRIM11-overexpressing THP-1 cells suppressing the AIM2 inflammasome (Liu et al., Cell Reports (2016) 16: 1988-2002), and other cell lines known in the art. Such AIM2 or NLRP3 knock-out reporter lines can express one or more inducible secreted reporter genes, such as Lucia luciferase and SEAP (secreted embryonic alkaline phosphatase). The reporter gene can be under the control of an ISG54 (interferon-stimulated gene) minimal promoter in conjunction with one or more, such as five, IFN-stimulated response elements. The reporter gene can also be under the control of an IFN-β minimal promoter fused to one or more, such as five, copies of a response element, such as an NF-kB response element. NLRP3 or AIM2 or caspase-1 activity in the presence of at least one inhibitor of NLRP3 or AIM2 or caspase-1 in combination with the ceDNAs described herein can be compared in the knock-out cell line versus the parental cell line.

In order to examine the effects of co-administration of a ceDNA of interest and an inhibitor of the NLRP3 inflammasome and/or an inhibitor of the AIM2 inflammasome, or a NLRP3 antagonist or an AIM2 antagonist on NLRP3 and/or AIM2 inflammasome pathway activation ex vivo, human monocytes can be isolated by, for example, gradient density centrifugation of peripheral blood and magnetic separation. These monocytes can be examined before and after contact with and/or activation with a ceDNA of interest with or without an inhibitor of the NLRP3 inflammasome and/or an inhibitor of the AIM2 inflammasome, or a NLRP3 antagonist or an AIM2 antagonist, or caspase-1 inhibitor with suitable controls. After treatment, serum and cell supernatants are used for measuring one or more cytokine pathways as a functional readout of activation of the NLRP3 inflammasome pathway and/or an inhibitor of the AIM2 inflammasome pathway, such as interleukin (IL)-1β, IL-6, IL-8, IL-18, interferon (IFN)-γ, interferon (IFN)-α, monocyte chemoattractant protein (MCP)-1, IP-10, and/or tumor necrosis factor (TNF)-α, using any assay or method known to a skilled artisan. In addition, nuclear extracts can be used to verify activation of NF-κB, using any assay or method known to a skilled artisan. It is expected that in the presence of an inhibitor of the NLRP3 and/or AIM2 inflammasome pathway, or a caspase 1 inhibitor, less activation of cytokine pathways and cytokine secretion is observed when administering a ceDNA, facilitating increased transgene expression duration and therapeutic efficacy.

In order to examine the effects of co-administration of a ceDNA of interest and an inhibitor of the NLRP3 and/or AIM2 inflammasome pathway, or a caspase 1 inhibitor on NLRP3 and/or AIM2 inflammasome pathway activation, or caspase 1 activation in vivo, a mouse model can be used. Serum or lymphocyte samples from the mouse are examined before and after contact with and/or activation with a ceDNA expressing a transgene of interest, such as Factor IX, with or without an inhibitor of the NLRP3 and/or AIM2 inflammasome pathway, or a caspase 1 inhibitor, with suitable controls. After treatment, serum and cell supernatants are used for measuring one or more cytokine pathways as a functional readout of activation of the NLRP3 and/or AIM2 inflammasome pathway, or a caspase 1 activation, such as interleukin (IL)-1β, IL-6, IL-8, IL-18, interferon (IFN)-γ, interferon (IFN)-α, monocyte chemoattractant protein (MCP)-1, and/or tumor necrosis factor (TNF)-α, using any assay or method known to a skilled artisan. In addition, nuclear extracts can be used to verify activation of NF-κB, using any assay or method known to a skilled artisan. It is expected that in the presence of an inhibitor of the NLRP3 and/or AIM2 inflammasome pathway, or a caspase 1 inhibitor, less immune activation and cytokine secretion is observed when administering a ceDNA, facilitating increased transgene expression duration and therapeutic efficacy.

Co-administration of a ceDNA of interest expressing human Factor IX produced from the plasmid TTX-9 and an inhibitor of the NLRP3 inflammasome and caspase-1 was assessed. Groups of C57bL mice (n=8) were assessed as shown in Table 11.

TABLE 11

| | | CeDNA | |
|---|---|---|---|
| | | Dose | |
| LNP | Group | Level | Immunosupression |
| LNP | 1 | siRNA 0.5 | none |
| | | mg/kg | |
| | 2 | 0.5 mg/kg | none |
| | | TTX9 | |
| | 3 | 0.5 mg/kg | Cremophor (Solvent control) by oral |
| | | TTX9 | gavage 12-16 hours before TTX9 |
| | | | and then one hour before TTX9. |
| | 4 | 0.5 mg/kg | VX765 100 mg/kg in Cremphor |
| | | TTX9 | 12-16 hours before TTX9 by oral |
| | | | gavage and then one hour before dose |
| | 5 | 0.5 mg/kg | MCC950 (NLRP3 inhibitor) 50 mg/kg |
| | | TTX9 | IP 12-16 hours before TTX9 and |
| | | | then one hour prior to ceDNA |

In brief, animals were pre-treated with an inhibitor of macrophage activation or a control according to the groups as shown in Table 12. Animals were administered MCC950 (NLRP2 inhibitor) (Group 5) or VX765 (Belnacasan; a selective caspase-1 inhibitor) (Group 4) i.p. 12-16 hours and then also 1 hour prior to administering 0.5 mg/kg ceDNA (TTX9-LNP) (Group 1) or LNP-siRNA (negative control) (Group 1) by IV administration via the lateral tail vein. A Pre-treatment control group was administered clondronate only (solvent control) (Group 3). Whole blood was collected via tail vein or facial vein or orbital bleed from each group on days 0, 1, 7 and 21.

TABLE 12
Pre-treatment Administration

| | ANI- | | | | |
|---|---|---|---|---|---|
| | MALS | PRE- | | | TREATMENT |
| GROUP | PER | TREAT- | DOSE | DOSE | REGIMEN, |
| NO. | GROUP | MENT | LEVEL | VOLUME | ROA |
| 1 | 8 | NA | NA | NA | NA |
| 2 | 8 | NA | NA | NA | |
| 3 | 8 | SOLVENT | 0.0 | 5 | ONCE ON |
| | | CONTROL | MG/KG | ML/KG | DAYS- |
| 4 | 8 | VX765 | 200 | | 1^{A} AND 0^{B} |
| | | | MG/KG | | BY PO |
| 5 | 8 | MCC950 | 50 | 5 | IP @ 1 HOUR |
| | | (NLRP3 | MG/KG | ML/KG | PRIOR |
| | | INHIBITOR) | | | TO TA |

^{A}First PO administration will occur 12-16 hours prior to ceDNA treatment on Day 0.
^{B}Second PO administration will occur 1 hour prior to ceDNA treatment on Day 0.
No. = Number;
ROA = route of administration;
PO = oral gavage;
IP = intraperitoneal;
IV = intravenous;
TA = test article;
NA = not applicable.

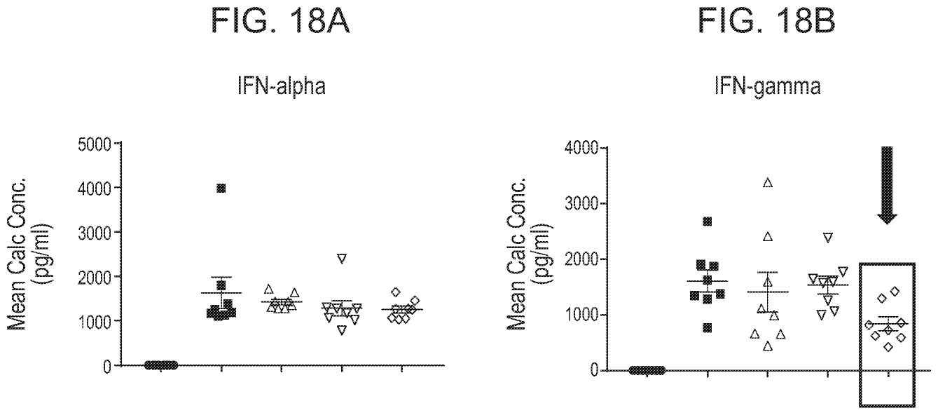

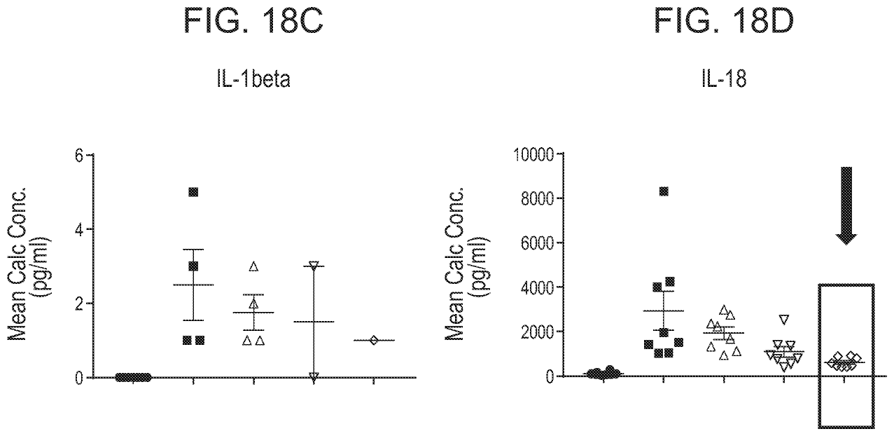

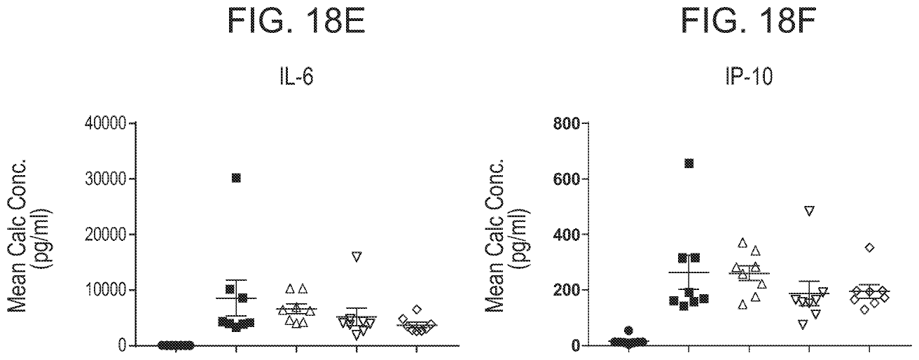

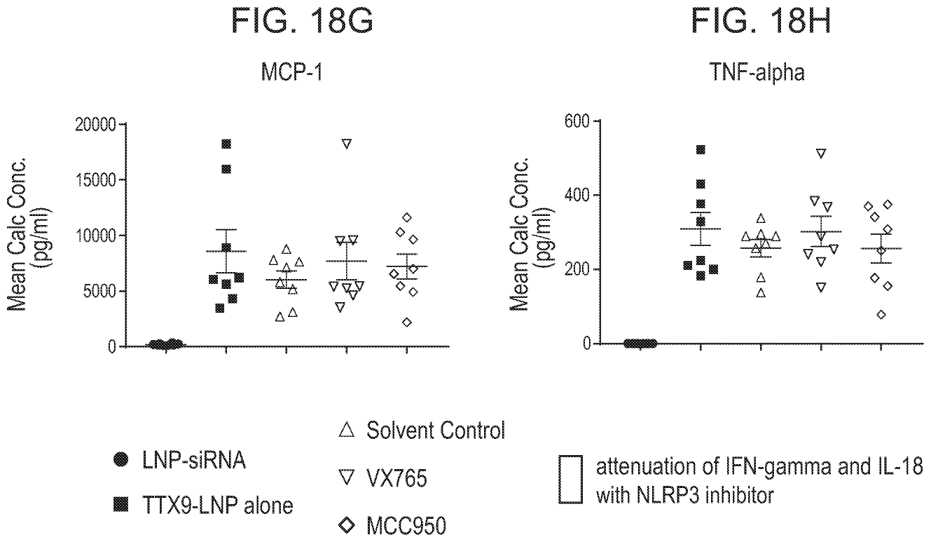

Cytokine levels were quantified and assessed using ProcartaPlex Multiplex Immunoassay (Invitrogen) according to the manufacturer instructions, which is a quantitative multiplex bead-based immunoassay for measuring levels of various cytokines and chemokines using the Luminex technology platform. Samples obtained from the study mice were mixed with pre-mixed custom mouse cytokine 8-plex kit, magnetic beads and assayed for levels of IFN-α, IFN-γ, IL-6, IP-10, IL-18, IL-1β, MCP-1, and TNF-alpha. In FIGS. 18A-18H, cytokine levels after TTX-9 administration with pharmacologic macrophage depletion with a NLRP3 inhibitor (MCC950) or Caspase 1 inhibitor (VX765) were assessed. Levels of IFNγ and IL-18 were significantly reduced with MCC950 (NLRP3 inhibitor) treatment (FIG. 18B and FIG. 18D), with a reduction in levels of IP-10 with MCC950 (FIG. 18F). Levels of IL-18 were also reduced with VX765 (caspase-1 inhibitor) (FIG. 18D).

Informal Sequence Listing

| Sequence | Description | Sequence |
|---|---|---|
| SEQ ID NO: | WT-ITR of | AGGAACCCCTAGTGATGGAGTTGGCCACTCCCTCTCTGCGCGCTCGCTCGCTCACTGAGGCCGGGCGACCAAA |
| 1 | AAV2 (Right) | GGTCGCCCGACGCCCGGGCTTTGCCCGGGCGGCCTCAGTGAGCGAGCGAGCGCGCAGCTGCCTGCAGG |
| SEQ ID NO: | Modified-ITR of | AGGAACCCCTAGTGATGGAGTTGGCCACTCCCTCTCTGCGCGCTCGCTCGCTCACTGAGGCCGGGCGACCAAA |
| 2 | AAV2 (Right) | GGTCGCCCGACGCCCGGGCGGCCTCAGTGAGCGAGCGAGCGCGCAGCTGCCTGCAGG |
| SEQ ID NO: | CAG promoter | TCAATATTGGCCATTAGCCATATTATTCATTGGTTATATAGCATAAATCAATATTGGCTATTGGCCATTGCATA |
| 3 | | CGTTGTATCTATATCATAATATGTACATTTATATTGGCTCATGTCCAATATGACCGCCATGTTGGCATTGATTA |
| | | TTGACTAGTTATTAATAGTAATCAATTACGGGGTCATTAGTTCATAGCCCATATATGGAGTTCCGCGTTACATA |
| | | ACTTACGGTAAATGGCCCGCCTGGCTGACCGCCCAACGACCCCCGCCCATTGACGTCAATAATGACGTATGTT |
| | | CCCATAGTAACGCCAATAGGGACTTTCCATTGACGTCAATGGGTGGAGTATTTACGGTAAACTGCCCACTTGG |
| | | CAGTACATCAAGTGTATCATATGCCAAGTCCGCCCCCTATTGACGTCAATGACGGTAAATGGCCCGCCTGGCA |
| | | TTATGCCCAGTACATGACCTTACGGGACTTTCCTACTTGGCAGTACATCTACGTATTAGTCATCGCTATTACCA |
| | | TGGTCGAGGTGAGCCCCACGTTCTGCTTCACTCTCCCCATCTCCCCCCCCTCCCCACCCCCAATTTTGTATTTA |
| | | TTTATTTTTTAATTATTTTGTGCAGCGATGGGGGCGGGGGGGGGGGGGGGGCGCGCGCCAGGCGGGGCGGGG |
| | | CGGGGCGAGGGGCGGGGCGGGGCGAGGCGGAGAGGTGCGGCGGCAGCCAATCAGAGCGGCGCGCTCCGAA |
| | | AGTTTCCTTTTATGGCGAGGCGGCGGCGGCGGCGGCCCTATAAAAAGCGAAGCGCGCGGCGGGCGGGAGTCG |
| | | CTGCGACGCTGCCTTCGCCCCGTGCCCCGCTCCGCCGCCGCCTCGCGCCGCCCGCCCCGGCTCTGACTGACCG |
| | | CGTTACTCCCACAGGTGAGCGGGCGGGACGGCCCTTCTCCTCCGGGCTGTAATTAGCGCTTGGTTTAATGACG |
| | | GCTTGTTTCTTTTCTGTGGCTGCGTGAAAGCCTTGAGGGGCTCCGGGAGGGCCCTTTGTGCGGGGGGGAGCGG |
| | | CTCGGGGGGTGCGTGCGTGTGTGTGTGCGTGGGGAGCGCCGCGTGCGGCCCGCGCTGCCCGGCGGCTGTGAG |
| | | CGCTGCGGGCGCGGCGCGGGGCTTTGTGCGCTCCGCAGTGTGCGCGAGGGGAGCGCGGCCGGGGGCGGTGCC |
| | | CCGCGGTGCGGGGGGGGCTGCGAGGGGAACAAAGGCTGCGTGCGGGGTGTGTGCGTGGGGGGGTGAGCAGG |
| | | GGGTGTGGGCGCGGCGGTCGGGCTGTAACCCCCCCCTGCACCCCCCTCCCCGAGTTGCTGAGCACGGCCCGG |
| | | CTTCGGGTGCGGGGCTCCGTACGGGGCGTGGCGCGGGGCTCGCCGTGCCGGGCGGGGGGTGGCGGCAGGTGG |
| | | GGGTGCCGGGCGGGGCGGGGCCGCCTCGGGCCGGGGAGGGCTCGGGGGAGGGGCGCGGCGGCCCCCGGAGC |
| | | GCCGGCGGCTGTCGAGGCGCGGCGAGCCGCAGCCATTGCCTTTTATGGTAATCGTGCGAGAGGGCGCAGGGA |
| | | CTTCCTTTGTCCCAAATCTGTGCGGAGCCGAAATCTGGGAGGCGCCGCCGCACCCCCTCTAGCGGGCGCGGGG |
| | | CGAAGCGGTGCGGCGCCGGCAGGAAGGAAATGGGCGGGGAGGGCCTTCGTGCGTCGCCGCGCCGCCGTCCC |
| | | CTTCTCCCTCTCCAGCCTCGGGGCTGTCCGCGGGGGGACGGCTGCCTTCGGGGGGGACGGGGCAGGGCGGGG |
| | | TTCGGCTTCTGGCGTGTGACCGGCGGCTCTAGAGCCTCTGCTAACCATGTTTTAGCCTTCTTCTTTTTCCTACA |
| | | GCTCCTGGGCAACGTGCTGGTTATTGTGCTGTCTCATCATTTGTCGACAGAATTCCTCGAAGATCCGAAGGGG |
| | | TTCAAGCTTGGCATTCCGGTACTGTTGGTAAAGCCA |
| SEQ ID NO: | AAT promoter | AGGCTCAGAGGCACACAGGAGTTTCTGGGCTCACCCTGCCCCCTTCCAACCCCTCAGTTCCCATCCTCCAGCA |
| 4 | | GCTGTTTGTGTGCTGCCTCTGAAGTCCACACTGAACAAACTTCAGCCTACTCATGTCCCTAAAATGGGCAAAC |
| | | ATTGCAAGCAGCAAACAGCAAACACACAGCCCTCCCTGCCTGCTGACCTTGGAGCTGGGGCAGAGGTCAGAG |
| | | ACCTCTCTGGGCCCATGCCACCTCCAACATCCACTCGACCCCTTGGAATTTCGGTGGAGAGGAGCAGAGGTTG |
| | | TCCTGGCGTGGTTTAGGTAGTGTGAGAGGGTCCGGGTTCAAAACCACTTGCTGGGTGGGGAGTCGTCAGTAA |
| | | GTGGCTATGCCCCGACCCCGAAGCCTGTTTCCCCATCTGTACAATGGAAATGATAAAGACGCCCATCTGATAG |
| | | GGTTTTTGTGGCAAATAAACATTTGGTTTTTTTGTTTTGTTTTGTTTTGTTTTTTGAGATGGAGGTTTGCTCTGT |
| | | CGCCCAGGCTGGAGTGCAGTGACACAATCTCATCTCACCACAACCTTCCCCTGCCTCAGCCTCCCAAGTAGCT |
| | | GGGATTACAAGCATGTGCCACCACACCTGGCTAATTTTCTATTTTTAGTAGAGACGGGTTTCTCCATGTTGGTC |
| | | AGCCTCAGCCTCCCAAGTAACTGGGATTACAGGCCTGTGCCACCACACCCGGCTAATTTTTTCTATTTTTGACA |
| | | GGGACGGGGTTTCACCATGTTGGTCAGGCTGGTCTAGAGGTACCGGATCTTGCTACCAGTGGAACAGCCACT |
| | | AAGGATTCTGCAGTGAGAGCAGAGGGCCAGCTAAGTGGTACTCTCCCAGAGACTGTCTGACTCACGCCACCC |
| | | CCTCCACCTTGGACACAGGACGCTGTGGTTTCTGAGCCAGGTACAATGACTCCTTTCGGTAAGTGCAGTGGAA |
| | | GCTGTACACTGCCCAGGCAAAGCGTCCGGGCAGCGTAGGCGGGCGACTCAGATCCCAGCCAGTGGACTTAGC |
| | | CCCTGTTTGCTCCTCCGATAACTGGGGTGACCTTGGTTAATATTCACCAGCAGCCTCCCCCGTTGCCCCTCTGG |
| | | ATCCACTGCTTAAATACGGACGAGGACAGGGCCCTGTCTCCTCAGCTTCAGGCACCACCACTGACCTGGGAC |
| | | AGTGAATCCGGACTCTAAGGTAAATATAAAATTTTTAAGTGTATAATGTGTTAAACTACTGATTCTAATTGTTT |
| | | CTCTCTTTTAGATTCCAACCTTTGGAACTGA |
| SEQ ID NO: | LP1 promoter | CCCTAAAATGGGCAAACATTGCAAGCAGCAAACAGCAAACACACAGCCCTCCCTGCCTGCTGACCTTGGAGC |
| 5 | | TGGGGCAGAGGTCAGAGACCTCTCTGGGCCCATGCCACCTCCAACATCCACTCGACCCCTTGGAATTTTTCGG |
| | | TGGAGAGGAGCAGAGGTTGTCCTGGCGTGGTTTAGGTAGTGTGAGAGGGGAATGACTCCTTTCGGTAAGTGC |
| | | AGTGGAAGCTGTACACTGCCCAGGCAAAGCGTCCGGGCAGCGTAGGCGGGCGACTCAGATCCCAGCCAGTGG |
| | | ACTTAGCCCCTGTTTGCTCCTCCGATAACTGGGGTGACCTTGGTTAATATTCACCAGCAGCCTCCCCCGTTGCC |
| | | CCTCTGGATCCACTGCTTAAATACGGACGAGGACAGGGCCCTGTCTCCTCAGCTTCAGGCACCACCACTGACC |
| | | TGGGACAGTGAATCCGGACTCTAAGGTAAATATAAAATTTTTAAGTGTATAATGTGTTAAACTACTGATTCTA |
| | | ATTGTTTCTCTCTTTTAGATTCCAACCTTTGGAACTGA |
| SEQ ID NO: | EF1-α promoter | GGCTCCGGTGCCCGTCAGTGGGCAGAGCGCACATCGCCCACAGTCCCCGAGAAGTTGGGGGGAGGGGTCGGC |
| 6 | | AATTGAACCGGTGCCTAGAGAAGGTGGCGCGGGGTAAACTGGGAAAGTGATGTCGTGTACTGGCTCCGCCTT |
| | | TTTCCCGAGGGTGGGGGAGAACCGTATATAAGTGCAGTAGTCGCCGTGAACGTTCTTTTTCGCAACGGGTTTG |
| | | CCGCCAGAACACAGGTAAGTGCCGTGTGTGGTTCCCGCGGGCCTGGCCTCTTTACGGGTTATGGCCCTTGCGT |
| | | GCCTTGAATTACTTCCACCTGGCTGCAGTACGTGATTCTTGATCCCGAGCTTCGGGTTGGAAGTGGGTGGGAG |
| | | AGTTCGAGGCCTTGCGCTTAAGGAGCCCCTTCGCCTCGTGCTTGAGTTGAGGCCTGGCCTGGGCGCTGGGGCC |
| | | GCCGCGTGCGAATCTGGTGGCACCTTCGCGCCTGTCTCGCTGCTTTCGATAAGTCTCTAGCCATTTAAAATTTT |
| | | TGATGACCTGCTGCGACGCTTTTTTTCTGGCAAGATAGTCTTGTAAATGCGGGCCAAGATCTGCACACTGGTA |
| | | TTTCGGTTTTTGGGGCCGCGGGCGGCGACGGGGCCCGTGCGTCCCAGCGCACATGTTCGGCGAGGCGGGGCC |
| | | TGCGAGCGCGGCCACCGAGAATCGGACGGGGGTAGTCTCAAGCTGGCCGGCCTGCTCTGGTGCCTGGTCTCG |
| | | CGCCGCCGTGTATCGCCCCGCCCTGGGCGGCAAGGCTGGCCCGGTCGGCACCAGTTGCGTGAGCGGAAAGAT |
| | | GGCCGCTTCCCGGCCCTGCTGCAGGGAGCTCAAAATGGAGGACGCGGCGCTCGGGAGAGCGGGCGGGTGAG |
| | | TCACCCACACAAAGGAAAAGGGCCTTTCCGTCCTCAGCCGTCGCTTCATGTGACTCCACGGAGTACCGGGCGC |
| | | CGTCCAGGCACCTCGATTAGTTCTCGAGCTTTTGGAGTACGTCGTCTTTAGGTTGGGGGGAGGGGTTTTATGC |
| | | GATGGAGTTTCCCCACACTGAGTGGGTGGAGACTGAAGTTAGGCCAGCTTGGCACTTGATGTAATTCTCCTTG |
| | | GAATTTGCCCTTTTTGAGTTTGGATCTTGGTTCATTCTCAAGCCTCAGACAGTGGTTCAAAGTTTTTTTCTTCCA |
| | | TTTCAGGTGTCGTGA |
| SEQ ID NO: | R3/R4 | R3 (PmeI) GTTTAAAC ; R4 (PacI) TTAATTAA |
| 7 | | |
| SEQ ID NO: | WPRE | GAGCATCTTACCGCCATTTATTCCCATATTTGTTCTGTTTTTCTTGATTTGGGTATACATTTAAATGTTAATAAA |
| 8 | | ACAAAATGGTGGGGCAATCATTTACATTTTTAGGGATATGTAATTACTAGTTCAGGTGTATTGCCACAAGACA |
| | | AACATGTTAAGAAACTTTCCCGTTATTTACGCTCTGTTCCTGTTAATCAACCTCTGGATTACAAAATTTGTGAA |
| | | AGATTGACTGATATTCTTAACTATGTTGCTCCTTTTACGCTGTGTGGATATGCTGCTTTATAGCCTCTGTATCTA |
| | | GCTATTGCTTCCCGTACGGCTTTCGTTTTCTCCTCCTTGTATAAATCCTGGTTGCTGTCTCTTTTAGAGGAGTTG |
| | | TGGCCCGTTGTCCGTCAACGTGGCGTGGTGTGCTCTGTGTTTGCTGACGCAACCCCCACTGGCTGGGGCATTG |
| | | CCACCACCTGTCAACTCCTTTCTGGGACTTTCGCTTTCCCCCTCCCGATCGCCACGGCAGAACTCATCGCCGCC |
| | | TGCCTTGCCCGCTGCTGGACAGGGGCTAGGTTGCTGGGCACTGATAATTCCGTGGTGTTGTC |
| SEQ ID NO: | BGHpA | TGTGCCTTCTAGTTGCCAGCCATCTGTTGTTTGCCCCTCCCCCGTGCCTTCCTTGACCCTGGAAGGTGCCACTC |
| 9 | | CCACTGTCCTTTCCTAATAAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTGGGGATGCGGTGGGCTC |
| | | TATGGC |
| SEQ ID NO: | Modified SV40- | TAAGATACATTGATGAGTTTGGACAAACCACAACTAGAATGCAGTGAAAAAAATGCTTTATTTGTGAAATTTG |
| 10 | pA | TGATGCTATTGCTTTATTTGTAACCATTATAAGCTGCAATAAACAAGTTAACAACAACAATTGCATTCATTTTA |
| | | TGTTTCAGGTTCAGGGGGAGGTGTGGGAGGTTTTTTAAAGCAAGTAAAACCTCTACAAATGTGGTA |
| SEQ ID NO: | wtFIX | ATGCAGCGCGTGAACATGATCATGGCCGAGAGCCCCGGCCTGATCACCATCTGCCTGCTGGGCTACCTGCTGA |
| 11 | | GCGCCGAGTGCACCGTGTTCCTGGACCACGAGAACGCCAACAAGATCCTGAACCGCCCCAAGCGCTACAACA |
| | | GCGGCAAGCTGGAGGAGTTCGTGCAGGGCAACCTGGAGCGCGAGTGCATGGAGGAGAAGTGCAGCTTCGAG |
| | | GAGGCCCGCGAGGTGTTCGAGAACACCGAGCGCACCACCGAGTTCTGGAAGCAGTACGTGGACGGCGACCA |
| | | GTGCGAGAGCAACCCCTGCCTGAACGGCGGCAGCTGCAAGGACGACATCAACAGCTACGAGTGCTGGTGCCC |
| | | CTTCGGCTTCGAGGGCAAGAACTGCGAGCTGGACGTGACCTGCAACATCAAGAACGGCCGCTGCGAGCAGTT |
| | | CTGCAAGAACAGCGCCGACAACAAGGTGGTGTGCAGCTGCACCGAGGGCTACCGCCTGGCCGAGAACCAGA |
| | | AGAGCTGCGAGCCCGCCGTGCCCTTCCCCTGCGGCAGGGTGAGCGTGAGCCAGACCAGCAAGCTGACCCGCG |
| | | CCGAGGCCGTGTTCCCCGACGTGGACTACGTGAACAGCACCGAGGCCGAGACCATCCTGGACAACATCACCC |
| | | AGAGCACCCAGAGCTTCAACGACTTCACCCGCGTGGTGGGCGGCGAGGACGCCAAGCCCGGCCAGTTCCCCT |
| | | GGCAGGTGGTGCTGAACGGCAAGGTGGACGCCTTCTGCGGCGGCAGCATCGTGAACGAGAAGTGGATCGTGA |
| | | CCGCCGCCCACTGCGTGGAGACCGGCGTGAAGATCACCGTGGTGGCCGGCGAGCACAACATCGAGGAGACC |
| | | GAGCACACCGAGCAGAAGCGCAACGTGATCCGCATCATCCCCCACCACAACTACAACGCCGCCATCAACAAG |
| | | TACAACCACGACATCGCCCTGCTGGAGCTGGACGAGCCCCTGGTGCTGAACAGCTACGTGACCCCCATCTGC |
| | | ATCGCCGACAAGGAGTACACCAACATCTTCCTGAAGTTCGGCAGCGGCTACGTGAGCGGCTGGGGCCGCGTG |
| | | TTCCACAAGGGCCGCAGCGCCCTGGTGCTGCAGTACCTGCGCGTGCCCCTGGTGGACCGCGCCACCTGCCTGC |
| | | GCAGCACCAAGTTCACCATCTACAACAACATGTTCTGCGCCGGCTTCCACGAGGGCGGCAGGGACAGCTGCC |
| | | AGGGCGACAGCGGCGGCCCCCACGTGACCGAGGTGGAGGGCACCAGCTTCCTGACCGGCATCATCAGCTGGG |
| | | GCGAGGAGTGCGCCATGAAGGGCAAGTACGGCATCTACACCAAGGTGAGCCGCTACGTGAACTGGATCAAG |
| | | GAGAAGACCAAGCTGACCTAA |
| | | Note: Sequence was subsequently codon optimized by GenScript. |
| SEQ ID NO: | PaduaFIX | ATGCAGCGCGTGAACATGATCATGGCCGAGAGCCCCGGCCTGATCACCATCTGCCTGCTGGGCTACCTGCTGA |
| 12 | | GCGCCGAGTGCACCGTGTTCCTGGACCACGAGAACGCCAACAAGATCCTGAACCGCCCCAAGCGCTACAACA |
| | | GCGGCAAGCTGGAGGAGTTCGTGCAGGGCAACCTGGAGCGCGAGTGCATGGAGGAGAAGTGCAGCTTCGAG |
| | | GAGGCCCGCGAGGTGTTCGAGAACACCGAGCGCACCACCGAGTTCTGGAAGCAGTACGTGGACGGCGACCA |
| | | GTGCGAGAGCAACCCCTGCCTGAACGGCGGCAGCTGCAAGGACGACATCAACAGCTACGAGTGCTGGTGCCC |
| | | CTTCGGCTTCGAGGGCAAGAACTGCGAGCTGGACGTGACCTGCAACATCAAGAACGGCCGCTGCGAGCAGTT |
| | | CTGCAAGAACAGCGCCGACAACAAGGTGGTGTGCAGCTGCACCGAGGGCTACCGCCTGGCCGAGAACCAGA |
| | | AGAGCTGCGAGCCCGCCGTGCCCTTCCCCTGCGGCAGGGTGAGCGTGAGCCAGACCAGCAAGCTGACCCGCG |
| | | CCGAGGCCGTGTTCCCCGACGTGGACTACGTGAACAGCACCGAGGCCGAGACCATCCTGGACAACATCACCC |
| | | AGAGCACCCAGAGCTTCAACGACTTCACCCGCGTGGTGGGCGGCGAGGACGCCAAGCCCGGCCAGTTCCCCT |
| | | GGCAGGTGGTGCTGAACGGCAAGGTGGACGCCTTCTGCGGCGGCAGCATCGTGAACGAGAAGTGGATCGTGA |
| | | CCGCCGCCCACTGCGTGGAGACCGGCGTGAAGATCACCGTGGTGGCCGGCGAGCACAACATCGAGGAGACC |
| | | GAGCACACCGAGCAGAAGCGCAACGTGATCCGCATCATCCCCCACCACAACTACAACGCCGCCATCAACAAG |
| | | TACAACCACGACATCGCCCTGCTGGAGCTGGACGAGCCCCTGGTGCTGAACAGCTACGTGACCCCCATCTGC |
| | | ATCGCCGACAAGGAGTACACCAACATCTTCCTGAAGTTCGGCAGCGGCTACGTGAGCGGCTGGGGCCGCGTG |
| | | TTCCACAAGGGCCGCAGCGCCCTGGTGCTGCAGTACCTGCGCGTGCCCCTGGTGGACCGCGCCACCTGCCTGC |
| | | TGAGCACCAAGTTCACCATCTACAACAACATGTTCTGCGCCGGCTTCCACGAGGGCGGCAGGGACAGCTGCC |
| | | AGGGCGACAGCGGCGGCCCCCACGTGACCGAGGTGGAGGGCACCAGCTTCCTGACCGGCATCATCAGCTGGG |
| | | GCGAGGAGTGCGCCATGAAGGGCAAGTACGGCATCTACACCAAGGTGAGCCGCTACGTGAACTGGATCAAG |
| | | GAGAAGACCAAGCTGACCTAA |
| | | Note: Sequence was subsequently codon optimized by GenScript. |
| SEQ ID NO: | Rep 78 | CGCAGCCACC |
| 13 | | ATGGCGGGGTTTTACGAGATTGTGATTAAGGTCCCCAGCGACCTTGACGGGCATCTGCCCGGCATTTCTGACA |
| | | GCTTTGTGAACTGGGTGGCCGAGAAGGAATGGGAGTTGCCGCCAGATTCTGACATGGATCTGAATCTGATTG |
| | | AGCAGGCACCCCTGACCGTGGCCGAGAAGCTGCAGCGCGACTTTCTGACGGAATGGCGCCGTGTGAGTAAGG |
| | | CCCCGGAGGCCCTTTTCTTTGTGCAATTTGAGAAGGGAGAGAGCTACTTCCACATGCACGTGCTCGTGGAAAC |
| | | CACCGGGGTGAAATCCATGGTTTTGGGACGTTTCCTGAGTCAGATTCGCGAAAAACTGATTCAGAGAATTTAC |
| | | CGCGGGATCGAGCCGACTTTGCCAAACTGGTTCGCGGTCACAAAGACCAGAAATGGCGCCGGAGGCGGGAA |
| | | CAAGGTGGTGGATGAGTGCTACATCCCCAATTACTTGCTCCCCAAAACCCAGCCTGAGCTCCAGTGGGCGTGG |
| | | ACTAATATGGAACAGTATTTAAGCGCCTGTTTGAATCTCACGGAGCGTAAACGGTTGGTGGCGCAGCATCTGA |
| | | CGCACGTGTCGCAGACGCAGGAGCAGAACAAAGAGAATCAGAATCCCAATTCTGATGCGCCGGTGATCAGAT |
| | | CAAAAACTTCAGCCAGGTACATGGAGCTGGTCGGGTGGCTCGTGGACAAGGGGATTACCTCGGAGAAGCAGT |
| | | GGATCCAGGAGGACCAGGCCTCATACATCTCCTTCAATGCGGCCTCCAACTCGCGGTCCCAAATCAAGGCTGC |
| | | CTTGGACAATGCGGGAAAGATTATGAGCCTGACTAAAACCGCCCCCGACTACCTGGTGGGCCAGCAGCCCGT |
| | | GGAGGACATTTCCAGCAATCGGATTTATAAAATTTTGGAACTAAACGGGTACGATCCCCAATATGCGGCTTCC |
| | | GTCTTTCTGGGATGGGCCACGAAAAAGTTCGGCAAGAGGAACACCATCTGGCTGTTTGGGCCTGCAACTACC |
| | | GGGAAGACCAACATCGCGGAGGCCATAGCCCACACTGTGCCCTTCTACGGGTGCGTAAACTGGACCAATGAG |
| | | AACTTTCCCTTCAACGACTGTGTCGACAAGATGGTGATCTGGTGGGAGGAGGGGAAGATGACCGCCAAGGTC |
| | | GTGGAGTCGGCCAAAGCCATTCTCGGAGGAAGCAAGGTGCGCGTGGACCAGAAATGCAAGTCCTCGGCCCAG |
| | | ATAGACCCGACTCCCGTGATCGTCACCTCCAACACCAACATGTGCGCCGTGATTGACGGGAACTCAACGACCT |
| | | TCGAACACCAGCAGCCGTTGCAAGACCGGATGTTCAAATTTGAACTCACCCGCCGTCTGGATCATGACTTTGG |
| | | GAAGGTCACCAAGCAGGAAGTCAAAGACTTTTTCCGGTGGGCAAAGGATCACGTGGTTGAGGTGGAGCATGA |
| | | ATTCTACGTCAAAAAGGGTGGAGCCAAGAAAAGACCCGCCCCCAGTGACGCAGATATAAGTGAGCCCAAAC |
| | | GGGTGCGCGAGTCAGTTGCGCAGCCATCGACGTCAGACGCGGAAGCTTCGATCAACTACGCAGACAGGTACC |
| | | AAAACAAATGTTCTCGTCACGTGGGCATGAATCTGATGCTGTTTCCCTGCAGACAATGCGAGAGAATGAATC |
| | | AGAATTCAAATATCTGCTTCACTCACGGACAGAAAGACTGTTTAGAGTGCTTTCCCGTGTCAGAATCTCAACC |
| | | CGTTTCTGTCGTCAAAAAGGCGTATCAGAAACTGTGCTACATTCATCATATCATGGGAAAGGTGCCAGACGCT |
| | | TGCACTGCCTGCGATCTGGTCAATGTGGATTTGGATGACTGCATCTTTGAACAATAA |
| SEQ ID NO: | Rep 52 | ATGGAGCTGGTCGGGTGGCTCGTGGACAAGGGGATTACCTCGGAGAAGCAGTGGATCCAGGAGGACCAGGC |
| 14 | | CTCATACATCTCCTTCAATGCGGCCTCCAACTCGCGGTCCCAAATCAAGGCTGCCTTGGACAATGCGGGAAAG |
| | | ATTATGAGCCTGACTAAAACCGCCCCCGACTACCTGGTGGGCCAGCAGCCCGTGGAGGACATTTCCAGCAAT |
| | | CGGATTTATAAAATTTTGGAACTAAACGGGTACGATCCCCAATATGCGGCTTCCGTCTTTCTGGGATGGGCCA |
| | | CGAAAAAGTTCGGCAAGAGGAACACCATCTGGCTGTTTGGGCCTGCAACTACCGGGAAGACCAACATCGCGG |
| | | AGGCCATAGCCCACACTGTGCCCTTCTACGGGTGCGTAAACTGGACCAATGAGAACTTTCCCTTCAACGACTG |
| | | TGTCGACAAGATGGTGATCTGGTGGGAGGAGGGGAAGATGACCGCCAAGGTCGTGGAGTCGGCCAAAGCCA |
| | | TTCTCGGAGGAAGCAAGGTGCGCGTGGACCAGAAATGCAAGTCCTCGGCCCAGATAGACCCGACTCCCGTGA |
| | | TCGTCACCTCCAACACCAACATGTGCGCCGTGATTGACGGGAACTCAACGACCTTCGAACACCAGCAGCCGTT |
| | | GCAAGACCGGATGTTCAAATTTGAACTCACCCGCCGTCTGGATCATGACTTTGGGAAGGTCACCAAGCAGGA |
| | | AGTCAAAGACTTTTTCCGGTGGGCAAAGGATCACGTGGTTGAGGTGGAGCATGAATTCTACGTCAAAAAGGG |
| | | TGGAGCCAAGAAAAGACCCGCCCCCAGTGACGCAGATATAAGTGAGCCCAAACGGGTGCGCGAGTCAGTTG |
| | | CGCAGCCATCGACGTCAGACGCGGAAGCTTCGATCAACTACGCAGACCGCTACCAAAACAAATGTTCTCGTC |
| | | ACGTGGGCATGAATCTGATGCTGTTTCCCTGCAGACAATGCGAGAGAATGAATCAGAATTCAAATATCTGCTT |
| | | CACTCACGGACAGAAAGACTGTTTAGAGTGCTTTCCCGTGTCAGAATCTCAACCCGTTTCTGTCGTCAAAAAG |
| | | GCGTATCAGAAACTGTGCTACATTCATCATATCATGGGAAAGGTGCCAGACGCTTGCACTGCCTGCGATCTGG |
| | | TCAATGTGGATTTGGATGACTGCATCTTTGAACAATAA |
| SEQ ID NO: | IE1 promoter | AATAAACGATAACGCCGTTGGTGGCGTGAGGCATGTAAAAGGTTACATCATTATCTTGTTCGCCATCCGGTTG |
| 15 | fragment | GTATAAATAGACGTTCATGTTGGTTTTTGTTTCAGTTGCAAGTTGGCTGCGGCGCGCGCAGCACCTTT |
| SEQ ID NO: | LP-1 β promoter | CCCTAAAATGGGCAAACATTGCAAGCAGCAAACAGCAAACACACAGCCCTCCCTGCCTGCTGACCTTGGAGC |
| 16 | | TGGGGCAGAGGTCAGAGACCTCTCTGGGCCCATGCCACCTCCAACATCCACTCGACCCCTTGGAATTTCGGTG |
| | | GAGAGGAGCAGAGGTTGTCCTGGCGTGGTTTAGGTAGTGTGAGAGGGGAATGACTCCTTTCGGTAAGTGCAG |
| | | TGGAAGCTGTACACTGCCCAGGCAAAGCGTCCGGGCAGCGTAGGCGGGCGACTCAGATCCCAGCCAGTGGAC |
| | | TTAGCCCCTGTTTGCTCCTCCGATAACTGGGGTGACCTTGGTTAATATTCACCAGCAGCCTCCCCCGTTGCCCC |
| | | TCTGGATCCACTGCTTAAATACGGACGAGGACACTCGAGGGCCCTGTCTCCTCAGCTTCAGGCACCACCACTG |
| | | ACCTGGGACAGTGAATCCGGACATCGATTCTAAGGTAAATATAAAATTTTTAAGTGTATAATTTGTTAAACTA |
| | | CTGATTCTAATTGTTTCTCTCTTTTAGATTCCAACCTTTGGAACTGA |
| SEQ ID NO: | Selected portion | GCGCGCTCGCTCGCTCACTGAGGCCGGGCGACCAAAGGTCGCCCGACGCCCGGGCGGCCTCAGTGAGCGAGCGAGCGCGC |
| 17 | of SEQ ID NO: 2 | |
| | Containing RBE | |
| SEQ ID NO: | RNA polymerase | GAGGGCCTATTTCCCATGATTCCTTCATATTTGCATATACGATACAAGGCTGTTAGAGAGATAATTGGAATTA |
| 18 | III promoter for | ATTTGACTGTAAACACAAAGATATTAGTACAAAATACGTGACGTAGAAAGTAATAATTTCTTGGGTAGTTTGC |
| | human U6 | AGTTTTAAAATTATGTTTTAAAATGGACTATCATATGCTTACCGTAACTTGAAAGTATTTCGATTTCTTGGCTT |
| | snRNA | TATATATCTTGTGGAAAGGAC |
| | (Human U6 | |
| | small nuclear | |
| | promoter) | |
| SEQ ID NO: | human H1 RNA | GAACGCTGACGTCATCAACCCGCTCCAAGGAATCGCGGGCCCAGTGTCACTAGGCGGGAACACCCAGCGCGC |
| 19 | promoter | GTGCGCCCTGGCAGGAAGATGGCTGTGAGGGACAGGGGAGTGGCGCCCTGCAATATTTGCATGTCGCTATGT |
| | (Human H1 | GTTCTGGGAAATCACCATAAACGTGAAATGTCTTTGGATTTGGGAATCGTATAAGAACTGTATGAGACCAC |
| | promoter) | |
| SEQ ID NO: | IE2 Promoter | ATAAACGATAACGCCGTTGGTGGCGTGAGGCATGTAAAAGGTTACATCATTATCTTGTTCGCCATCCGGTTGG |
| 20 | | TATAAATAGACGTTCATGTTGGTTTTTGTTTCAGTTGCAAGTTGGCTGCGGCGCGCGCAGCACCTTTGCGGCC |
| | | ATCT |
| SEQ ID NO: | | |
| 21-38 | | |
| SEQ ID NO: | Rep-binding site | GCGCGCTCGCTCGCTC |
| 39 | (RBS) for AAV2 | |
| SEQ ID NO: | | |
| 40-50 | | |
| SEQ ID NO: | WT-ITR of | CCTGCAGGCAGCTGCGCGCTCGCTCGCTCACTGAGGCCGCCCGGGCAAAGCCCGGGCGTCGGGCGACCTTTG |
| 51 | AAV2 (Left) | GTCGCCCGGCCTCAGTGAGCGAGCGAGCGCGCAGAGAGGGAGTGGCCAACTCCATCACTAGGGGTTCCT |
| SEQ ID NO: | Modified-ITR of | CCTGCAGGCAGCTGCGCGCTCGCTCGCTCACTGAGGCCGCCCGGGCGTCGGGCGACCTTTGGTCGCCCGGCCT |
| 52 | AAV2 (Left) | CAGTGAGCGAGCGAGCGCGCAGAGAGGGAGTGGCCAACTCCATCACTAGGGGTTCCT |
| SEQ ID NO: | Construct A | CCTGCAGGCAGCTGCGCGCTCGCTCGCTCACTGAGGCCGCCCGGGAAACCCGGGCGTGCCCGGGCGCCTCAG |
| 53 | | TGAGCGAGCGAGCGCGCAGAGAGGGAGTGGCCAACTCCATCACTAGGGGTTCCTGAACAGAGAAACAGGAG |
| | | AATATGGGCCAAACAGGATATCTGTGGTAAGCAGTTCCTGCCCCGGCTCAGGGCCAAGAACAGTTGGAACAG |
| | | CAGAATATGGGCCAAACAGGATATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTTCCAGGGTGCCCCAAGGACCTGAA |
| | | ATGACCCTGTGCCTTATTTGAACTAACCAATCAGTTCGCTTCTCGCTTCTGTTCGCGCGCTTCTGCTCCCCGAG |
| | | CTCTATATAAGCAGAGCTCGTTTAGTGAACCGTCAGATCGCCTGGAGACGCCATCCACGCTGTTTTGACTTCC |
| | | ATAGAAGGCCGCCACCATGGAAGACGCCAAAAACATAAAGAAAGGCCCGGCGCCATTCTATCCGCTGGAAG |
| | | ATGGAACCGCTGGAGAGCAACTGCATAAGGCTATGAAGAGATACGCCCTGGTTCCTGGAACAATTGCTTTTA |
| | | CAGATGCACATATCGAGGTGGACATCACTTACGCTGAGTACTTCGAAATGTCCGTTCGGTTGGCAGAAGCTAT |
| | | GAAACGATATGGGCTGAATACAAATCACAGAATCGTCGTATGCAGTGAAAACTCTCTTCAATTCTTTATGCCG |
| | | GTGTTGGGCGCGTTATTTATCGGAGTTGCAGTTGCGCCCGCGAACGACATTTATAATGAACGTGAATTGCTCA |
| | | ACAGTATGGGCATTTCGCAGCCTACCGTGGTGTTCGTTTCCAAAAAGGGGTTGCAAAAAATTTTGAACGTGCA |
| | | AAAAAAGCTCCCAATCATCCAAAAAATTATTATCATGGATTCTAAAACGGATTACCAGGGATTTCAGTCGATG |
| | | TACACGTTCGTCACATCTCATCTACCTCCCGGTTTTAATGAATACGATTTTGTGCCAGAGTCCTTCGATAGGGA |
| | | CAAGACAATTGCACTGATCATGAACTCCTCTGGATCTACTGGTCTGCCTAAAGGTGTCGCTCTGCCTCATAGA |
| | | ACTGCCTGCGTGAGATTCTCGCATGCCAGAGATCCTATTTTTGGCAATCAAATCATTCCGGATACTGCGATTTT |
| | | AAGTGTTGTTCCATTCCATCACGGTTTTGGAATGTTTACTACACTCGGATATTTGATATGTGGATTTCGAGTCG |
| | | TCTTAATGTATAGATTTGAAGAAGAGCTGTTTCTGAGGAGCCTTCAGGATTACAAGATTCAAAGTGCGCTGCT |
| | | GGTGCCAACCCTATTCTCCTTCTTCGCCAAAAGCACTCTGATTGACAAATACGATTTATCTAATTTACACGAA |
| | | ATTGCTTCTGGTGGCGCTCCCCTCTCTAAGGAAGTCGGGGAAGCGGTTGCCAAGAGGTTCCATCTGCCAGGTA |
| | | TCAGGCAAGGATATGGGCTCACTGAGACTACATCAGCTATTCTGATTACACCCGAGGGGGATGATAAACCGG |
| | | GCGCGGTCGGTAAAGTTGTTCCATTTTTTGAAGCGAAGGTTGTGGATCTGGATACCGGGAAAACGCTGGGCGT |
| | | TAATCAAAGAGGCGAACTGTGTGTGAGAGGTCCTATGATTATGTCCGGTTATGTAAACAATCCGGAAGCGAC |
| | | CAACGCCTTGATTGACAAGGATGGATGGCTACATTCTGGAGACATAGCTTACTGGGACGAAGACGAACACTT |
| | | CTTCATCGTTGACCGCCTGAAGTCTCTGATTAAGTACAAAGGCTATCAGGTGGCTCCCGCTGAATTGGAATCC |
| | | ATCTTGCTCCAACACCCCAACATCTTCGACGCAGGTGTCGCAGGTCTTCCCGACGATGACGCCGGTGAACTTC |
| | | CCGCCGCCGTTGTTGTTTTGGAGCACGGAAAGACGATGACGGAAAAAGAGATCGTGGATTACGTCGCCAGTC |
| | | AAGTAACAACCGCGAAAAAGTTGCGCGGAGGAGTTGTGTTTGTGGACGAAGTACCGAAAGGTCTTACCGGAA |
| | | AACTCGACGCAAGAAAAATCAGAGAGATCCTCATAAAGGCCAAGAAGGGCGGAAAGATCGCCGTGTAAGAG |
| | | CATCTTACCGCCATTTATTCCCATATTTGTTCTGTTTTTCTTGATTTGGGTATACATTTAAATGTTAATAAAACA |
| | | AAATGGTGGGGCAATCATTTACATTTTTAGGGATATGTAATTACTAGTTCAGGTGTATTGCCACAAGACAAAC |
| | | ATGTTAAGAAACTTTCCCGTTATTTACGCTCTGTTCCTGTTAATCAACCTCTGGATTACAAAATTTGTGAAAGA |
| | | TTGACTGATATTCTTAACTATGTTGCTCCTTTTACGCTGTGTGGATATGCTGCTTTATAGCCTCTGTATCTAGCT |
| | | ATTGCTTCCCGTACGGCTTTCGTTTTCTCCTCCTTGTATAAATCCTGGTTGCTGTCTCTTTTAGAGGAGTTGTGG |
| | | CCCGTTGTCCGTCAACGTGGCGTGGTGTGCTCTGTGTTTGCTGACGCAACCCCCACTGGCTGGGGCATTGCCA |
| | | CCACCTGTCAACTCCTTTCTGGGACTTTCGCTTTCCCCCTCCCGATCGCCACGGCAGAACTCATCGCCGCCTGC |
| | | CTTGCCCGCTGCTGGACAGGGGCTAGGTTGCTGGGCACTGATAATTCCGTGGTGTTGTCTGTGCCTTCTAGTTG |
| | | CCAGCCATCTGTTGTTTGCCCCTCCCCCGTGCCTTCCTTGACCCTGGAAGGTGCCACTCCCACTGTCCTTTCCT |
| | | AATAAAATGAGGAAATTGCATCGCATTGTCTGAGTAGGTGTCATTCTATTCTGGGGGGTGGGGTGGGGCAGG |
| | | ACAGCAAGGGGGAGGATTGGGAAGACAATAGCAGGCATGCTGGGGATGCGGTGGGCTCTATGGCAGGAACC |
| | | CCTAGTGATGGAGTTGGCCACTCCCTCTCTGCGCGCTCGCTCGCTCACTGAGGCCGGGCGACCAAAGGTCGCC |
| | | CGACGCCCGGGCTTTGCCCGGGCGGCCTCAGTGAGCGAGCGAGCGCGCAGCTGCCTGCAGG |
| SEQ ID NO: | Construct B | CCTGCAGGCAGCTGCGCGCTCGCTCGCTCACTGAGGCCGCCCGGGCAAAGCCCGGGCGTCGGGCGACCTTTG |
| 54 | | GTCGCCCGGCCTCAGTGAGCGAGCGAGCGCGCAGAGAGGGAGTGGCCAACTCCATCACTAGGGGTTCCTGAA |
| | | CAGAGAAACAGGAGAATATGGGCCAAACAGGATATCTGTGGTAAGCAGTTCCTGCCCCGGCTCAGGGCCAAG |
| | | AACAGTTGGAACAGCAGAATATGGGCCAAACAGGATATCTGTGGTAAGCAGTTCCTGCCCCGGCTCAGGGCC |
| | | AAGAACAGATGGTCCCCAGATGCGGTCCCGCCCTCAGCAGTTTCTAGAGAACCATCAGATGTTTCCAGGGTG |
| | | CCCCAAGGACCTGAAATGACCCTGTGCCTTATTTGAACTAACCAATCAGTTCGCTTCTCGCTTCTGTTCGCGCG |
| | | CTTCTGCTCCCCGAGCTCTATATAAGCAGAGCTCGTTTAGTGAACCGTCAGATCGCCTGGAGACGCCATCCAC |
| | | GCTGTTTTGACTTCCATAGAAGGCCGCCACCATGGAAGACGCCAAAAACATAAAGAAAGGCCCGGCGCCATT |
| | | CTATCCGCTGGAAGATGGAACCGCTGGAGAGCAACTGCATAAGGCTATGAAGAGATACGCCCTGGTTCCTGG |
| | | AACAATTGCTTTTACAGATGCACATATCGAGGTGGACATCACTTACGCTGAGTACTTCGAAATGTCCGTTCGG |
| | | TTGGCAGAAGCTATGAAACGATATGGGCTGAATACAAATCACAGAATCGTCGTATGCAGTGAAAACTCTCTT |
| | | CAATTCTTTATGCCGGTGTTGGGCGCGTTATTTATCGGAGTTGCAGTTGCGCCCGCGAACGACATTTATAATG |
| | | AACGTGAATTGCTCAACAGTATGGGCATTTCGCAGCCTACCGTGGTGTTCGTTTCCAAAAAGGGGTTGCAAAA |
| | | AATTTTGAACGTGCAAAAAAAGCTCCCAATCATCCAAAAAATTATTATCATGGATTCTAAAACGGATTACCAG |
| | | GGATTTCAGTCGATGTACACGTTCGTCACATCTCATCTACCTCCCGGTTTTAATGAATACGATTTTGTGCCAGA |
| | | GTCCTTCGATAGGGACAAGACAATTGCACTGATCATGAACTCCTCTGGATCTACTGGTCTGCCTAAAGGTGTC |
| | | GCTCTGCCTCATAGAACTGCCTGCGTGAGATTCTCGCATGCCAGAGATCCTATTTTTGGCAATCAAATCATTCC |
| | | GGATACTGCGATTTTAAGTGTTGTTCCATTCCATCACGGTTTTGGAATGTTTACTACACTCGGATATTTGATAT |
| | | GTGGATTTCGAGTCGTCTTAATGTATAGATTTGAAGAAGAGCTGTTTCTGAGGAGCCTTCAGGATTACAAGAT |
| | | TCAAAGTGCGCTGCTGGTGCCAACCCTATTCTCCTTCTTCGCCAAAAGCACTCTGATTGACAAATACGATTTAT |
| | | CTAATTTACACGAAATTGCTTCTGGTGGCGCTCCCCTCTCTAAGGAAGTCGGGGAAGCGGTTGCCAAGAGGTT |
| | | CCATCTGCCAGGTATCAGGCAAGGATATGGGCTCACTGAGACTACATCAGCTATTCTGATTACACCCGAGGG |
| | | GGATGATAAACCGGGCGCGGTCGGTAAAGTTGTTCCATTTTTTGAAGCGAAGGTTGTGGATCTGGATACCGGG |
| | | AAAACGCTGGGCGTTAATCAAAGAGGCGAACTGTGTGTGAGAGGTCCTATGATTATGTCCGGTTATGTAAAC |
| | | AATCCGGAAGCGACCAACGCCTTGATTGACAAGGATGGATGGCTACATTCTGGAGACATAGCTTACTGGGAC |
| | | GAAGACGAACACTTCTTCATCGTTGACCGCCTGAAGTCTCTGATTAAGTACAAAGGCTATCAGGTGGCTCCCG |
| | | CTGAATTGGAATCCATCTTGCTCCAACACCCCAACATCTTCGACGCAGGTGTCGCAGGTCTTCCCGACGATGA |
| | | CGCCGGTGAACTTCCCGCCGCCGTTGTTGTTTTGGAGCACGGAAAGACGATGACGGAAAAAGAGATCGTGGA |
| | | TTACGTCGCCAGTCAAGTAACAACCGCGAAAAAGTTGCGCGGAGGAGTTGTGTTTGTGGACGAAGTACCGAA |
| | | AGGTCTTACCGGAAAACTCGACGCAAGAAAAATCAGAGAGATCCTCATAAAGGCCAAGAAGGGCGGAAAGA |
| | | TCGCCGTGTAAGAGCATCTTACCGCCATTTATTCCCATATTTGTTCTGTTTTTCTTGATTTGGGTATACATTTAA |
| | | ATGTTAATAAAACAAAATGGTGGGGCAATCATTTACATTTTTAGGGATATGTAATTACTAGTTCAGGTGTATT |
| | | GCCACAAGACAAACATGTTAAGAAACTTTCCCGTTATTTACGCTCTGTTCCTGTTAATCAACCTCTGGATTACA |
| | | AAATTTGTGAAAGATTGACTGATATTCTTAACTATGTTGCTCCTTTTACGCTGTGTGGATATGCTGCTTTATAG |
| | | CCTCTGTATCTAGCTATTGCTTCCCGTACGGCTTTCGTTTTCTCCTCCTTGTATAAATCCTGGTTGCTGTCTCTT |
| | | TTAGAGGAGTTGTGGCCCGTTGTCCGTCAACGTGGCGTGGTGTGCTCTGTGTTTGCTGACGCAACCCCCACTG |
| | | GCTGGGGCATTGCCACCACCTGTCAACTCCTTTCTGGGACTTTCGCTTTCCCCCTCCCGATCGCCACGGCAGAA |
| | | CTCATCGCCGCCTGCCTTGCCCGCTGCTGGACAGGGGCTAGGTTGCTGGGCACTGATAATTCCGTGGTGTTGT |
| | | CTGTGCCTTCTAGTTGCCAGCCATCTGTTGTTTGCCCCTCCCCCGTGCCTTCCTTGACCCTGGAAGGTGCCACT |
| | | CCCACTGTCCTTTCCTAATAAAATGAGGAAATTGCATCGCATTGTCTGAGTAGGTGTCATTCTATTCTGGGGG |
| | | GTGGGGTGGGGCAGGACAGCAAGGGGGAGGATTGGGAAGACAATAGCAGGCATGCTGGGGATGCGGTGGGC |
| | | TCTATGGCAGGAACCCCTAGTGATGGAGTTGGCCACTCCCTCTCTGCGCGCTCGCTCGCTCACTGAGGCCGCC |
| | | CGGGAAACCCGGGCGTGCGCCTCAGTGAGCGAGCGAGCGCGCAGCTGCCTGCAGG |
| SEQ ID NO: | Construct C | CCTGCAGGCAGCTGCGCGCTCGCTCGCTCACTGAGGCCGCCCGGGAAACCCGGGCGTGCCCGGGCGCCTCAG |
| 55 | | TGAGCGAGCGAGCGCGCAGAGAGGGAGTGGCCAACTCCATCACTAGGGGTTCCTGAACAGAGAAACAGGAG |
| | | AATATGGGCCAAACAGGATATCTGTGGTAAGCAGTTCCTGCCCCGGCTCAGGGCCAAGAACAGTTGGAACAG |
| | | CAGAATATGGGCCAAACAGGATATCTGTGGTAAGCAGTTCCTGCCCCGGCTCAGGGCCAAGAACAGATGGTC |
| | | CCCAGATGCGGTCCCGCCCTCAGCAGTTTCTAGAGAACCATCAGATGTTTCCAGGGTGCCCCAAGGACCTGAA |
| | | ATGACCCTGTGCCTTATTTGAACTAACCAATCAGTTCGCTTCTCGCTTCTGTTCGCGCGCTTCTGCTCCCCGAG |
| | | CTCTATATAAGCAGAGCTCGTTTAGTGAACCGTCAGATCGCCTGGAGACGCCATCCACGCTGTTTTGACTTCC |
| | | ATAGAAGGCCGCCACCATGATCATGGCCGAGAGCCCTGGCCTGATCACCATCTGCCTGCTGGGCTACCTGCTG |
| | | AGCGCCGAGTGCACCGTGTTCCTGGACCACGAGAACGCCAACAAGATCCTGAACCGGCCCAAGAGATACAAC |
| | | AGCGGCAAGCTGGAGGAGTTCGTGCAGGGCAACCTGGAGAGGGAGTGCATGGAGGAGAAGTGCAGCTTCGA |
| | | GGAGGCCAGGGAAGTGTTCGAGAACACCGAGCGGACCACCGAGTTCTGGAAGCAGTACGTGGACGGCGACC |
| | | AGTGCGAGAGCAACCCTTGCCTGAACGGCGGCAGCTGCAAGGACGACATCAACAGCTACGAGTGCTGGTGCC |
| | | CTTTCGGCTTCGAGGGCAAGAACTGCGAGCTGGACGTGACCTGCAACATCAAGAACGGCCGCTGCGAGCAGT |
| | | TCTGCAAGAACAGCGCCGACAACAAAGTGGTGTGTAGCTGCACCGAGGGCTACAGACTGGCCGAGAACCAG |
| | | AAGAGCTGCGAGCCCGCCGTGCCCTTCCCCTGCGGCAGAGTGAGCGTGTCCCAGACCAGCAAGCTGACCAGA |
| | | GCCGAGACCGTGTTCCCCGACGTGGACTACGTGAATAGCACCGAGGCCGAGACCATCCTGGACAACATCACC |
| | | CAGAGCACCCAGTCCTTCAACGACTTCACCAGAGTTGTGGGCGGCGAGGACGCCAAGCCCGGCCAGTTCCCC |
| | | TGGCAGGTGGTGCTGAACGGCAAAGTGGATGCCTTCTGCGGCGGCAGCATCGTGAACGAGAAGTGGATCGTG |
| | | ACAGCCGCCCACTGCGTGGAGACCGGCGTGAAGATCACCGTGGTGGCCGGCGAACACAATATCGAGGAGAC |
| | | CGAGCACACCGAGCAGAAGCGGAACGTCATCCGGATTATCCCCCACCACAACTACAACGCCGCCATCAACAA |
| | | GTACAACCACGACATCGCCCTGCTGGAGCTGGACGAGCCTCTGGTGCTGAATAGCTACGTGACCCCCATCTGC |
| | | ATCGCCGACAAGGAGTACACCAACATCTTCCTGAAGTTCGGCAGCGGCTACGTGTCCGGCTGGGGCAGAGTG |
| | | TTCCACAAGGGCAGAAGCGCCCTGGTGCTGCAGTACCTGAGAGTGCCCCTGGTGGACAGAGCCACCTGCCTG |
| | | TTGAGCACCAAGTTCACCATCTACAACAACATGTTCTGCGCCGGCTTCCACGAGGGCGGCAGAGACAGCTGC |
| | | CAGGGCGACAGCGGCGGACCCCACGTGACCGAAGTGGAGGGCACCAGCTTCCTGACCGGCATCATCAGCTGG |
| | | GGCGAGGAGTGCGCCATGAAGGGCAAGTACGGCATCTACACCAAAGTGAGCCGGTACGTGAACTGGATCAA |
| | | GGAGAAAACCAAGCTGACCTGAGAGCATCTTACCGCCATTTATTCCCATATTTGTTCTGTTTTTCTTGATTTGG |
| | | GTATACATTTAAATGTTAATAAAACAAAATGGTGGGGCAATCATTTACATTTTTAGGGATATGTAATTACTAG |
| | | TTCAGGTGTATTGCCACAAGACAAACATGTTAAGAAACTTTCCCGTTATTTACGCTCTGTTCCTGTTAATCAAC |
| | | CTCTGGATTACAAAATTTGTGAAAGATTGACTGATATTCTTAACTATGTTGCTCCTTTTACGCTGTGTGGATAT |
| | | GCTGCTTTATAGCCTCTGTATCTAGCTATTGCTTCCCGTACGGCTTTCGTTTTCTCCTCCTTGTATAAATCCTGG |
| | | TTGCTGTCTCTTTTAGAGGAGTTGTGGCCCGTTGTCCGTCAACGTGGCGTGGTGTGCTCTGTGTTTGCTGACGC |
| | | AACCCCCACTGGCTGGGGCATTGCCACCACCTGTCAACTCCTTTCTGGGACTTTCGCTTTCCCCCTCCCGATCG |
| | | CCACGGCAGAACTCATCGCCGCCTGCCTTGCCCGCTGCTGGACAGGGGCTAGGTTGCTGGGCACTGATAATTC |
| | | CGTGGTGTTGTCTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGTGTCATTCT |
| | | ATTCTGGGGGGTGGGGTGGGGCAGGACAGCAAGGGGGAGGATTGGGAAGACAATAGCAGGCATGCTGGGGA |
| | | TGCGGTGGGCTCTATGGCAGGAACCCCTAGTGATGGAGTTGGCCACTCCCTCTCTGCGCGCTCGCTCGCTCAC |
| | | TGAGGCCGGGCGACCAAAGGTCGCCCGACGCCCGGGCTTTGCCCGGGCGGCCTCAGTGAGCGAGCGAGCGCG |
| | | CAGCTGCCTGCAGG |
| SEQ ID NO: | Construct D | CCTGCAGGCAGCTGCGCGCTCGCTCGCTCACTGAGGCCGCCCGGGCAAAGCCCGGGCGTCGGGCGACCTTTG |
| 56 | | GTCGCCCGGCCTCAGTGAGCGAGCGAGCGCGCAGAGAGGGAGTGGCCAACTCCATCACTAGGGGTTCCTGAA |
| | | CAGAGAAACAGGAGAATATGGGCCAAACAGGATATCTGTGGTAAGCAGTTCCTGCCCCGGCTCAGGGCCAAG |
| | | AACAGTTGGAACAGCAGAATATGGGCCAAACAGGATATCTGTGGTAAGCAGTTCCTGCCCCGGCTCAGGGCC |
| | | AAGAACAGATGGTCCCCAGATGCGGTCCCGCCCTCAGCAGTTTCTAGAGAACCATCAGATGTTTCCAGGGTG |
| | | CCCCAAGGACCTGAAATGACCCTGTGCCTTATTTGAACTAACCAATCAGTTCGCTTCTCGCTTCTGTTCGCGCG |
| | | CTTCTGCTCCCCGAGCTCTATATAAGCAGAGCTCGTTTAGTGAACCGTCAGATCGCCTGGAGACGCCATCCAC |
| | | GCTGTTTTGACTTCCATAGAAGGCCGCCACCATGATCATGGCCGAGAGCCCTGGCCTGATCACCATCTGCCTG |
| | | CTGGGCTACCTGCTGAGCGCCGAGTGCACCGTGTTCCTGGACCACGAGAACGCCAACAAGATCCTGAACCGG |
| | | CCCAAGAGATACAACAGCGGCAAGCTGGAGGAGTTCGTGCAGGGCAACCTGGAGAGGGAGTGCATGGAGGA |
| | | GAAGTGCAGCTTCGAGGAGGCCAGGGAAGTGTTCGAGAACACCGAGCGGACCACCGAGTTCTGGAAGCAGT |
| | | ACGTGGACGGCGACCAGTGCGAGAGCAACCCTTGCCTGAACGGCGGCAGCTGCAAGGACGACATCAACAGC |
| | | TACGAGTGCTGGTGCCCTTTCGGCTTCGAGGGCAAGAACTGCGAGCTGGACGTGACCTGCAACATCAAGAAC |
| | | GGCCGCTGCGAGCAGTTCTGCAAGAACAGCGCCGACAACAAAGTGGTGTGTAGCTGCACCGAGGGCTACAGA |
| | | CTGGCCGAGAACCAGAAGAGCTGCGAGCCCGCCGTGCCCTTCCCCTGCGGCAGAGTGAGCGTGTCCCAGACC |
| | | AGCAAGCTGACCAGAGCCGAGACCGTGTTCCCCGACGTGGACTACGTGAATAGCACCGAGGCCGAGACCATC |
| | | CTGGACAACATCACCCAGAGCACCCAGTCCTTCAACGACTTCACCAGAGTTGTGGGCGGCGAGGACGCCAAG |
| | | CCCGGCCAGTTCCCCTGGCAGGTGGTGCTGAACGGCAAAGTGGATGCCTTCTGCGGCGGCAGCATCGTGAAC |
| | | GAGAAGTGGATCGTGACAGCCGCCCACTGCGTGGAGACCGGCGTGAAGATCACCGTGGTGGCCGGCGAACA |
| | | CAATATCGAGGAGACCGAGCACACCGAGCAGAAGCGGAACGTCATCCGGATTATCCCCCACCACAACTACAA |
| | | CGCCGCCATCAACAAGTACAACCACGACATCGCCCTGCTGGAGCTGGACGAGCCTCTGGTGCTGAATAGCTA |
| | | CGTGACCCCCATCTGCATCGCCGACAAGGAGTACACCAACATCTTCCTGAAGTTCGGCAGCGGCTACGTGTCC |
| | | GGCTGGGGCAGAGTGTTCCACAAGGGCAGAAGCGCCCTGGTGCTGCAGTACCTGAGAGTGCCCCTGGTGGAC |
| | | AGAGCCACCTGCCTGTTGAGCACCAAGTTCACCATCTACAACAACATGTTCTGCGCCGGCTTCCACGAGGGCG |
| | | GCAGAGACAGCTGCCAGGGCGACAGCGGCGGACCCCACGTGACCGAAGTGGAGGGCACCAGCTTCCTGACC |
| | | GGCATCATCAGCTGGGGCGAGGAGTGCGCCATGAAGGGCAAGTACGGCATCTACACCAAAGTGAGCCGGTA |
| | | CGTGAACTGGATCAAGGAGAAAACCAAGCTGACCTGAGAGCATCTTACCGCCATTTATTCCCATATTTGTTCT |
| | | GTTTTTCTTGATTTGGGTATACATTTAAATGTTAATAAAACAAAATGGTGGGGCAATCATTTACATTTTTAGGG |
| | | ATATGTAATTACTAGTTCAGGTGTATTGCCACAAGACAAACATGTTAAGAAACTTTCCCGTTATTTACGCTCTG |
| | | TTCCTGTTAATCAACCTCTGGATTACAAAATTTGTGAAAGATTGACTGATATTCTTAACTATGTTGCTCCTTTT |
| | | ACGCTGTGTGGATATGCTGCTTTATAGCCTCTGTATCTAGCTATTGCTTCCCGTACGGCTTTCGTTTTCTCCTCC |
| | | TTGTATAAATCCTGGTTGCTGTCTCTTTTAGAGGAGTTGTGGCCCGTTGTCCGTCAACGTGGCGTGGTGTGCTC |
| | | TGTGTTTGCTGACGCAACCCCCACTGGCTGGGGCATTGCCACCACCTGTCAACTCCTTTCTGGGACTTTCGCTT |
| | | TCCCCCTCCCGATCGCCACGGCAGAACTCATCGCCGCCTGCCTTGCCCGCTGCTGGACAGGGGCTAGGTTGCT |
| | | GGGCACTGATAATTCCGTGGTGTTGTCTGTGCCTTCTAGTTGCCAGCCATCTGTTGTTTGCCCCTCCCCCGTGC |
| | | CTTCCTTGACCCTGGAAGGTGCCACTCCCACTGTCCTTTCCTAATAAAATGAGGAAATTGCATCGCATTGTCTG |
| | | AGTAGGTGTCATTCTATTCTGGGGGGTGGGGTGGGGCAGGACAGCAAGGGGGAGGATTGGGAAGACAATAG |
| | | CAGGCATGCTGGGGATGCGGTGGGCTCTATGGCAGGAACCCCTAGTGATGGAGTTGGCCACTCCCTCTCTGCG |
| | | CGCTCGCTCGCTCACTGAGGCCGCCCGGGAAACCCGGGCGTGCGCCTCAGTGAGCGAGCGAGCGCGCAGCTG |
| | | CCTGCAGG |
| SEQ ID NO: | Construct E | CCTGCAGGCAGCTGCGCGCTCGCTCGCTCACTGAGGCCGCCCGGGAAACCCGGGCGTGCCCGGGCGCCTCAG |
| 57 | | TGAGCGAGCGAGCGCGCAGAGAGGGAGTGGCCAACTCCATCACTAGGGGTTCCTGGCTCAGAGGCTCAGAG |
| | | GCACACAGGAGTTTCTGGGCTCACCCTGCCCCCTTCCAACCCCTCAGTTCCCATCCTCCAGCAGCTGTTTGTGT |
| | | GCTGCCTCTGAAGTCCACACTGAACAAACTTCAGCCTACTCATGTCCCTAAAATGGGCAAACATTGCAAGCAG |
| | | CAAACAGCAAACACACAGCCCTCCCTGCCTGCTGACCTTGGAGCTGGGGCAGAGGTCAGAGACCTCTCTGGG |
| | | CCCATGCCACCTCCAACATCCACTCGACCCCTTGGAATTTCGGTGGAGAGGAGCAGAGGTTGTCCTGGCGTGG |
| | | TTTAGGTAGTGTGAGAGGGTCCGGGTTCAAAACCACTTGCTGGGTGGGGAGTCGTCAGTAAGTGGCTATGCC |
| | | CCGACCCCGAAGCCTGTTTCCCCATCTGTACAATGGAAATGATAAAGACGCCCATCTGATAGGGTTTTTGTGG |
| | | CAAATAAACATTTGGTTTTTTTGTTTTGTTTTGTTTTGTTTTTTGAGATGGAGGTTTGCTCTGTCGCCCAGGCTG |
| | | GAGTGCAGTGACACAATCTCATCTCACCACAACCTTCCCCTGCCTCAGCCTCCCAAGTAGCTGGGATTACAAG |
| | | CATGTGCCACCACACCTGGCTAATTTTCTATTTTTAGTAGAGACGGGTTTCTCCATGTTGGTCAGCCTCAGCCT |
| | | CCCAAGTAACTGGGATTACAGGCCTGTGCCACCACACCCGGCTAATTTTTTCTATTTTTGACAGGGACGGGGT |
| | | TTCACCATGTTGGTCAGGCTGGTCTAGAGGTACCGGATCTTGCTACCAGTGGAACAGCCACTAAGGATTCTGC |
| | | AGTGAGAGCAGAGGGCCAGCTAAGTGGTACTCTCCCAGAGACTGTCTGACTCACGCCACCCCCTCCACCTTG |
| | | GACACAGGACGCTGTGGTTTCTGAGCCAGGTACAATGACTCCTTTCGGTAAGTGCAGTGGAAGCTGTACACTG |
| | | CCCAGGCAAAGCGTCCGGGCAGCGTAGGCGGGCGACTCAGATCCCAGCCAGTGGACTTAGCCCCTGTTTGCT |
| | | CCTCCGATAACTGGGGTGACCTTGGTTAATATTCACCAGCAGCCTCCCCCGTTGCCCCTCTGGATCCACTGCTT |
| | | AAATACGGACGAGGACAGGGCCCTGTCTCCTCAGCTTCAGGCACCACCACTGACCTGGGACAGTGCCGCCAC |
| | | CATGGAAGACGCCAAAAACATAAAGAAAGGCCCGGCGCCATTCTATCCGCTGGAAGATGGAACCGCTGGAG |
| | | AGCAACTGCATAAGGCTATGAAGAGATACGCCCTGGTTCCTGGAACAATTGCTTTTACAGATGCACATATCGA |
| | | GGTGGACATCACTTACGCTGAGTACTTCGAAATGTCCGTTCGGTTGGCAGAAGCTATGAAACGATATGGGCTG |
| | | AATACAAATCACAGAATCGTCGTATGCAGTGAAAACTCTCTTCAATTCTTTATGCCGGTGTTGGGCGCGTTAT |
| | | TTATCGGAGTTGCAGTTGCGCCCGCGAACGACATTTATAATGAACGTGAATTGCTCAACAGTATGGGCATTTC |
| | | GCAGCCTACCGTGGTGTTCGTTTCCAAAAAGGGGTTGCAAAAAATTTTGAACGTGCAAAAAAAGCTCCCAAT |
| | | CATCCAAAAAATTATTATCATGGATTCTAAAACGGATTACCAGGGATTTCAGTCGATGTACACGTTCGTCACA |
| | | TCTCATCTACCTCCCGGTTTTAATGAATACGATTTTGTGCCAGAGTCCTTCGATAGGGACAAGACAATTGCAC |
| | | TGATCATGAACTCCTCTGGATCTACTGGTCTGCCTAAAGGTGTCGCTCTGCCTCATAGAACTGCCTGCGTGAG |
| | | ATTCTCGCATGCCAGAGATCCTATTTTTGGCAATCAAATCATTCCGGATACTGCGATTTTAAGTGTTGTTCCAT |
| | | TCCATCACGGTTTTGGAATGTTTACTACACTCGGATATTTGATATGTGGATTTCGAGTCGTCTTAATGTATAGA |
| | | TTTGAAGAAGAGCTGTTTCTGAGGAGCCTTCAGGATTACAAGATTCAAAGTGCGCTGCTGGTGCCAACCCTAT |
| | | TCTCCTTCTTCGCCAAAAGCACTCTGATTGACAAATACGATTTATCTAATTTACACGAAATTGCTTCTGGTGGC |
| | | GCTCCCCTCTCTAAGGAAGTCGGGGAAGCGGTTGCCAAGAGGTTCCATCTGCCAGGTATCAGGCAAGGATAT |
| | | GGGCTCACTGAGACTACATCAGCTATTCTGATTACACCCGAGGGGGATGATAAACCGGGCGCGGTCGGTAAA |
| | | GTTGTTCCATTTTTTGAAGCGAAGGTTGTGGATCTGGATACCGGGAAAACGCTGGGCGTTAATCAAAGAGGCG |
| | | AACTGTGTGTGAGAGGTCCTATGATTATGTCCGGTTATGTAAACAATCCGGAAGCGACCAACGCCTTGATTGA |
| | | CAAGGATGGATGGCTACATTCTGGAGACATAGCTTACTGGGACGAAGACGAACACTTCTTCATCGTTGACCG |
| | | CCTGAAGTCTCTGATTAAGTACAAAGGCTATCAGGTGGCTCCCGCTGAATTGGAATCCATCTTGCTCCAACAC |
| | | CCCAACATCTTCGACGCAGGTGTCGCAGGTCTTCCCGACGATGACGCCGGTGAACTTCCCGCCGCCGTTGTTG |
| | | TTTTGGAGCACGGAAAGACGATGACGGAAAAAGAGATCGTGGATTACGTCGCCAGTCAAGTAACAACCGCG |
| | | AAAAAGTTGCGCGGAGGAGTTGTGTTTGTGGACGAAGTACCGAAAGGTCTTACCGGAAAACTCGACGCAAGA |
| | | AAAATCAGAGAGATCCTCATAAAGGCCAAGAAGGGCGGAAAGATCGCCGTGTAAGAGCATCTTACCGCCATT |
| | | TATTCCCATATTTGTTCTGTTTTTCTTGATTTGGGTATACATTTAAATGTTAATAAAACAAAATGGTGGGGCAA |
| | | TCATTTACATTTTTAGGGATATGTAATTACTAGTTCAGGTGTATTGCCACAAGACAAACATGTTAAGAAACTTT |
| | | CCCGTTATTTACGCTCTGTTCCTGTTAATCAACCTCTGGATTACAAAATTTGTGAAAGATTGACTGATATTCTT |
| | | AACTATGTTGCTCCTTTTACGCTGTGTGGATATGCTGCTTTATAGCCTCTGTATCTAGCTATTGCTTCCCGTACG |
| | | GCTTTCGTTTTCTCCTCCTTGTATAAATCCTGGTTGCTGTCTCTTTTAGAGGAGTTGTGGCCCGTTGTCCGTCAA |
| | | CGTGGCGTGGTGTGCTCTGTGTTTGCTGACGCAACCCCCACTGGCTGGGGCATTGCCACCACCTGTCAACTCC |
| | | TTTCTGGGACTTTCGCTTTCCCCCTCCCGATCGCCACGGCAGAACTCATCGCCGCCTGCCTTGCCCGCTGCTGG |
| | | ACAGGGGCTAGGTTGCTGGGCACTGATAATTCCGTGGTGTTGTCTGTGCCTTCTAGTTGCCAGCCATCTGTTGT |
| | | TTGCCCCTCCCCCGTGCCTTCCTTGACCCTGGAAGGTGCCACTCCCACTGTCCTTTCCTAATAAAATGAGGAAA |
| | | TTGCATCGCATTGTCTGAGTAGGTGTCATTCTATTCTGGGGGGTGGGGTGGGGCAGGACAGCAAGGGGGAGG |
| | | ATTGGGAAGACAATAGCAGGCATGCTGGGGATGCGGTGGGCTCTATGGCAGGAACCCCTAGTGATGGAGTTG |
| | | GCCACTCCCTCTCTGCGCGCTCGCTCGCTCACTGAGGCCGGGCGACCAAAGGTCGCCCGACGCCCGGGCTTTG |
| | | CCCGGGCGGCCTCAGTGAGCGAGCGAGCGCGCAGCTGCCTGCAGG |
| SEQ ID NO: | Construct F | CCTGCAGGCAGCTGCGCGCTCGCTCGCTCACTGAGGCCGCCCGGGCAAAGCCCGGGCGTCGGGCGACCTTTG |
| 58 | | GTCGCCCGGCCTCAGTGAGCGAGCGAGCGCGCAGAGAGGGAGTGGCCAACTCCATCACTAGGGGTTCCTGGC |
| | | TCAGAGGCTCAGAGGCACACAGGAGTTTCTGGGCTCACCCTGCCCCCTTCCAACCCCTCAGTTCCCATCCTCC |
| | | AGCAGCTGTTTGTGTGCTGCCTCTGAAGTCCACACTGAACAAACTTCAGCCTACTCATGTCCCTAAAATGGGC |
| | | AAACATTGCAAGCAGCAAACAGCAAACACACAGCCCTCCCTGCCTGCTGACCTTGGAGCTGGGGCAGAGGTC |
| | | AGAGACCTCTCTGGGCCCATGCCACCTCCAACATCCACTCGACCCCTTGGAATTTCGGTGGAGAGGAGCAGA |
| | | GGTTGTCCTGGCGTGGTTTAGGTAGTGTGAGAGGGTCCGGGTTCAAAACCACTTGCTGGGTGGGGAGTCGTCA |
| | | GTAAGTGGCTATGCCCCGACCCCGAAGCCTGTTTCCCCATCTGTACAATGGAAATGATAAAGACGCCCATCTG |
| | | ATAGGGTTTTTGTGGCAAATAAACATTTGGTTTTTTTGTTTTGTTTTGTTTTGTTTTTTGAGATGGAGGTTTGCT |
| | | CTGTCGCCCAGGCTGGAGTGCAGTGACACAATCTCATCTCACCACAACCTTCCCCTGCCTCAGCCTCCCAAGT |
| | | AGCTGGGATTACAAGCATGTGCCACCACACCTGGCTAATTTTCTATTTTTAGTAGAGACGGGTTTCTCCATGTT |
| | | GGTCAGCCTCAGCCTCCCAAGTAACTGGGATTACAGGCCTGTGCCACCACACCCGGCTAATTTTTTCTATTTTT |
| | | GACAGGGACGGGGTTTCACCATGTTGGTCAGGCTGGTCTAGAGGTACCGGATCTTGCTACCAGTGGAACAGC |
| | | CACTAAGGATTCTGCAGTGAGAGCAGAGGGCCAGCTAAGTGGTACTCTCCCAGAGACTGTCTGACTCACGCC |
| | | ACCCCCTCCACCTTGGACACAGGACGCTGTGGTTTCTGAGCCAGGTACAATGACTCCTTTCGGTAAGTGCAGT |
| | | GGAAGCTGTACACTGCCCAGGCAAAGCGTCCGGGCAGCGTAGGCGGGCGACTCAGATCCCAGCCAGTGGACT |
| | | TAGCCCCTGTTTGCTCCTCCGATAACTGGGGTGACCTTGGTTAATATTCACCAGCAGCCTCCCCCGTTGCCCCT |
| | | CTGGATCCACTGCTTAAATACGGACGAGGACAGGGCCCTGTCTCCTCAGCTTCAGGCACCACCACTGACCTGG |
| | | GACAGTGCCGCCACCATGGAAGACGCCAAAAACATAAAGAAAGGCCCGGCGCCATTCTATCCGCTGGAAGAT |
| | | GGAACCGCTGGAGAGCAACTGCATAAGGCTATGAAGAGATACGCCCTGGTTCCTGGAACAATTGCTTTTACA |
| | | GATGCACATATCGAGGTGGACATCACTTACGCTGAGTACTTCGAAATGTCCGTTCGGTTGGCAGAAGCTATGA |
| | | AACGATATGGGCTGAATACAAATCACAGAATCGTCGTATGCAGTGAAAACTCTCTTCAATTCTTTATGCCGGT |
| | | GTTGGGCGCGTTATTTATCGGAGTTGCAGTTGCGCCCGCGAACGACATTTATAATGAACGTGAATTGCTCAAC |
| | | AGTATGGGCATTTCGCAGCCTACCGTGGTGTTCGTTTCCAAAAAGGGGTTGCAAAAAATTTTGAACGTGCAAA |
| | | AAAAGCTCCCAATCATCCAAAAAATTATTATCATGGATTCTAAAACGGATTACCAGGGATTTCAGTCGATGTA |
| | | CACGTTCGTCACATCTCATCTACCTCCCGGTTTTAATGAATACGATTTTGTGCCAGAGTCCTTCGATAGGGACA |
| | | AGACAATTGCACTGATCATGAACTCCTCTGGATCTACTGGTCTGCCTAAAGGTGTCGCTCTGCCTCATAGAAC |
| | | TGCCTGCGTGAGATTCTCGCATGCCAGAGATCCTATTTTTGGCAATCAAATCATTCCGGATACTGCGATTTTAA |
| | | GTGTTGTTCCATTCCATCACGGTTTTGGAATGTTTACTACACTCGGATATTTGATATGTGGATTTCGAGTCGTC |
| | | TTAATGTATAGATTTGAAGAAGAGCTGTTTCTGAGGAGCCTTCAGGATTACAAGATTCAAAGTGCGCTGCTGG |
| | | TGCCAACCCTATTCTCCTTCTTCGCCAAAAGCACTCTGATTGACAAATACGATTTATCTAATTTACACGAAATT |
| | | GCTTCTGGTGGCGCTCCCCTCTCTAAGGAAGTCGGGGAAGCGGTTGCCAAGAGGTTCCATCTGCCAGGTATCA |
| | | GGCAAGGATATGGGCTCACTGAGACTACATCAGCTATTCTGATTACACCCGAGGGGGATGATAAACCGGGCG |
| | | CGGTCGGTAAAGTTGTTCCATTTTTTGAAGCGAAGGTTGTGGATCTGGATACCGGGAAAACGCTGGGCGTTAA |
| | | TCAAAGAGGCGAACTGTGTGTGAGAGGTCCTATGATTATGTCCGGTTATGTAAACAATCCGGAAGCGACCAA |
| | | CGCCTTGATTGACAAGGATGGATGGCTACATTCTGGAGACATAGCTTACTGGGACGAAGACGAACACTTCTTC |
| | | ATCGTTGACCGCCTGAAGTCTCTGATTAAGTACAAAGGCTATCAGGTGGCTCCCGCTGAATTGGAATCCATCT |
| | | TGCTCCAACACCCCAACATCTTCGACGCAGGTGTCGCAGGTCTTCCCGACGATGACGCCGGTGAACTTCCCGC |
| | | CGCCGTTGTTGTTTTGGAGCACGGAAAGACGATGACGGAAAAAGAGATCGTGGATTACGTCGCCAGTCAAGT |
| | | AACAACCGCGAAAAAGTTGCGCGGAGGAGTTGTGTTTGTGGACGAAGTACCGAAAGGTCTTACCGGAAAACT |
| | | CGACGCAAGAAAAATCAGAGAGATCCTCATAAAGGCCAAGAAGGGCGGAAAGATCGCCGTGTAAGAGCATC |
| | | TTACCGCCATTTATTCCCATATTTGTTCTGTTTTTCTTGATTTGGGTATACATTTAAATGTTAATAAAACAAAAT |
| | | GGTGGGGCAATCATTTACATTTTTAGGGATATGTAATTACTAGTTCAGGTGTATTGCCACAAGACAAACATGT |
| | | TAAGAAACTTTCCCGTTATTTACGCTCTGTTCCTGTTAATCAACCTCTGGATTACAAAATTTGTGAAAGATTGA |
| | | CTGATATTCTTAACTATGTTGCTCCTTTTACGCTGTGTGGATATGCTGCTTTATAGCCTCTGTATCTAGCTATTG |
| | | CTTCCCGTACGGCTTTCGTTTTCTCCTCCTTGTATAAATCCTGGTTGCTGTCTCTTTTAGAGGAGTTGTGGCCCG |
| | | TTGTCCGTCAACGTGGCGTGGTGTGCTCTGTGTTTGCTGACGCAACCCCCACTGGCTGGGGCATTGCCACCAC |
| | | CTGTCAACTCCTTTCTGGGACTTTCGCTTTCCCCCTCCCGATCGCCACGGCAGAACTCATCGCCGCCTGCCTTG |
| | | CCCGCTGCTGGACAGGGGCTAGGTTGCTGGGCACTGATAATTCCGTGGTGTTGTCTGTGCCTTCTAGTTGCCA |
| | | GCCATCTGTTGTTTGCCCCTCCCCCGTGCCTTCCTTGACCCTGGAAGGTGCCACTCCCACTGTCCTTTCCTAAT |
| | | AAAATGAGGAAATTGCATCGCATTGTCTGAGTAGGTGTCATTCTATTCTGGGGGGTGGGGTGGGGCAGGACA |
| | | GCAAGGGGGAGGATTGGGAAGACAATAGCAGGCATGCTGGGGATGCGGTGGGCTCTATGGCAGGAACCCCT |
| | | AGTGATGGAGTTGGCCACTCCCTCTCTGCGCGCTCGCTCGCTCACTGAGGCCGCCCGGGAAACCCGGGCGTGC |
| | | GCCTCAGTGAGCGAGCGAGCGCGCAGCTGCCTGCAGG |
| SEQ ID NO: | Construct G | CCTGCAGGCAGCTGCGCGCTCGCTCGCTCACTGAGGCCGCCCGGGAAACCCGGGCGTGCCCGGGCGCCTCAG |
| 59 | | TGAGCGAGCGAGCGCGCAGAGAGGGAGTGGCCAACTCCATCACTAGGGGTTCCTGGCTCAGAGGCTCAGAG |
| | | GCACACAGGAGTTTCTGGGCTCACCCTGCCCCCTTCCAACCCCTCAGTTCCCATCCTCCAGCAGCTGTTTGTGT |
| | | GCTGCCTCTGAAGTCCACACTGAACAAACTTCAGCCTACTCATGTCCCTAAAATGGGCAAACATTGCAAGCAG |
| | | CAAACAGCAAACACACAGCCCTCCCTGCCTGCTGACCTTGGAGCTGGGGCAGAGGTCAGAGACCTCTCTGGG |
| | | CCCATGCCACCTCCAACATCCACTCGACCCCTTGGAATTTCGGTGGAGAGGAGCAGAGGTTGTCCTGGCGTGG |
| | | TTTAGGTAGTGTGAGAGGGTCCGGGTTCAAAACCACTTGCTGGGTGGGGAGTCGTCAGTAAGTGGCTATGCC |
| | | CCGACCCCGAAGCCTGTTTCCCCATCTGTACAATGGAAATGATAAAGACGCCCATCTGATAGGGTTTTTGTGG |
| | | CAAATAAACATTTGGTTTTTTTGTTTTGTTTTGTTTTGTTTTTTGAGATGGAGGTTTGCTCTGTCGCCCAGGCTG |
| | | GAGTGCAGTGACACAATCTCATCTCACCACAACCTTCCCCTGCCTCAGCCTCCCAAGTAGCTGGGATTACAAG |
| | | CATGTGCCACCACACCTGGCTAATTTTCTATTTTTAGTAGAGACGGGTTTCTCCATGTTGGTCAGCCTCAGCCT |
| | | CCCAAGTAACTGGGATTACAGGCCTGTGCCACCACACCCGGCTAATTTTTTCTATTTTTGACAGGGACGGGGT |
| | | TTCACCATGTTGGTCAGGCTGGTCTAGAGGTACCGGATCTTGCTACCAGTGGAACAGCCACTAAGGATTCTGC |
| | | AGTGAGAGCAGAGGGCCAGCTAAGTGGTACTCTCCCAGAGACTGTCTGACTCACGCCACCCCCTCCACCTTG |
| | | GACACAGGACGCTGTGGTTTCTGAGCCAGGTACAATGACTCCTTTCGGTAAGTGCAGTGGAAGCTGTACACTG |
| | | CCCAGGCAAAGCGTCCGGGCAGCGTAGGCGGGCGACTCAGATCCCAGCCAGTGGACTTAGCCCCTGTTTGCT |
| | | CCTCCGATAACTGGGGTGACCTTGGTTAATATTCACCAGCAGCCTCCCCCGTTGCCCCTCTGGATCCACTGCTT |
| | | AAATACGGACGAGGACAGGGCCCTGTCTCCTCAGCTTCAGGCACCACCACTGACCTGGGACAGTGCCGCCAC |
| | | CATGATCATGGCCGAGAGCCCTGGCCTGATCACCATCTGCCTGCTGGGCTACCTGCTGAGCGCCGAGTGCACC |
| | | GTGTTCCTGGACCACGAGAACGCCAACAAGATCCTGAACCGGCCCAAGAGATACAACAGCGGCAAGCTGGA |
| | | GGAGTTCGTGCAGGGCAACCTGGAGAGGGAGTGCATGGAGGAGAAGTGCAGCTTCGAGGAGGCCAGGGAAG |
| | | TGTTCGAGAACACCGAGCGGACCACCGAGTTCTGGAAGCAGTACGTGGACGGCGACCAGTGCGAGAGCAAC |
| | | CCTTGCCTGAACGGCGGCAGCTGCAAGGACGACATCAACAGCTACGAGTGCTGGTGCCCTTTCGGCTTCGAG |
| | | GGCAAGAACTGCGAGCTGGACGTGACCTGCAACATCAAGAACGGCCGCTGCGAGCAGTTCTGCAAGAACAG |
| | | CGCCGACAACAAAGTGGTGTGTAGCTGCACCGAGGGCTACAGACTGGCCGAGAACCAGAAGAGCTGCGAGC |
| | | CCGCCGTGCCCTTCCCCTGCGGCAGAGTGAGCGTGTCCCAGACCAGCAAGCTGACCAGAGCCGAGACCGTGT |
| | | TCCCCGACGTGGACTACGTGAATAGCACCGAGGCCGAGACCATCCTGGACAACATCACCCAGAGCACCCAGT |
| | | CCTTCAACGACTTCACCAGAGTTGTGGGCGGCGAGGACGCCAAGCCCGGCCAGTTCCCCTGGCAGGTGGTGC |
| | | TGAACGGCAAAGTGGATGCCTTCTGCGGCGGCAGCATCGTGAACGAGAAGTGGATCGTGACAGCCGCCCACT |
| | | GCGTGGAGACCGGCGTGAAGATCACCGTGGTGGCCGGCGAACACAATATCGAGGAGACCGAGCACACCGAG |
| | | CAGAAGCGGAACGTCATCCGGATTATCCCCCACCACAACTACAACGCCGCCATCAACAAGTACAACCACGAC |
| | | ATCGCCCTGCTGGAGCTGGACGAGCCTCTGGTGCTGAATAGCTACGTGACCCCCATCTGCATCGCCGACAAGG |
| | | AGTACACCAACATCTTCCTGAAGTTCGGCAGCGGCTACGTGTCCGGCTGGGGCAGAGTGTTCCACAAGGGCA |
| | | GAAGCGCCCTGGTGCTGCAGTACCTGAGAGTGCCCCTGGTGGACAGAGCCACCTGCCTGTTGAGCACCAAGT |
| | | TCACCATCTACAACAACATGTTCTGCGCCGGCTTCCACGAGGGCGGCAGAGACAGCTGCCAGGGCGACAGCG |
| | | GCGGACCCCACGTGACCGAAGTGGAGGGCACCAGCTTCCTGACCGGCATCATCAGCTGGGGCGAGGAGTGCG |
| | | CCATGAAGGGCAAGTACGGCATCTACACCAAAGTGAGCCGGTACGTGAACTGGATCAAGGAGAAAACCAAG |
| | | CTGACCTGAGAGCATCTTACCGCCATTTATTCCCATATTTGTTCTGTTTTTCTTGATTTGGGTATACATTTAAAT |
| | | GTTAATAAAACAAAATGGTGGGGCAATCATTTACATTTTTAGGGATATGTAATTACTAGTTCAGGTGTATTGC |
| | | CACAAGACAAACATGTTAAGAAACTTTCCCGTTATTTACGCTCTGTTCCTGTTAATCAACCTCTGGATTACAA |
| | | AATTTGTGAAAGATTGACTGATATTCTTAACTATGTTGCTCCTTTTACGCTGTGTGGATATGCTGCTTTATAGC |
| | | CTCTGTATCTAGCTATTGCTTCCCGTACGGCTTTCGTTTTCTCCTCCTTGTATAAATCCTGGTTGCTGTCTCTTTT |
| | | AGAGGAGTTGTGGCCCGTTGTCCGTCAACGTGGCGTGGTGTGCTCTGTGTTTGCTGACGCAACCCCCACTGGC |
| | | TGGGGCATTGCCACCACCTGTCAACTCCTTTCTGGGACTTTCGCTTTCCCCCTCCCGATCGCCACGGCAGAACT |
| | | CATCGCCGCCTGCCTTGCCCGCTGCTGGACAGGGGCTAGGTTGCTGGGCACTGATAATTCCGTGGTGTTGTCT |
| | | GTGCCTTCTAGTTGCCAGCCATCTGTTGTTTGCCCCTCCCCCGTGCCTTCCTTGACCCTGGAAGGTGCCACTCC |
| | | CACTGTCCTTTCCTAATAAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTGGGGATGCGGTGGGCTC |
| | | TATGGCAGGAACCCCTAGTGATGGAGTTGGCCACTCCCTCTCTGCGCGCTCGCTCGCTCACTGAGGCCGGGCG |
| | | ACCAAAGGTCGCCCGACGCCCGGGCTTTGCCCGGGCGGCCTCAGTGAGCGAGCGAGCGCGCAGCTGCCTGCA |
| | | GG |
| SEQ ID NO: | Construct H | CCTGCAGGCAGCTGCGCGCTCGCTCGCTCACTGAGGCCGCCCGGGCAAAGCCCGGGCGTCGGGCGACCTTTG |
| 60 | | GTCGCCCGGCCTCAGTGAGCGAGCGAGCGCGCAGAGAGGGAGTGGCCAACTCCATCACTAGGGGTTCCTGGC |
| | | TCAGAGGCTCAGAGGCACACAGGAGTTTCTGGGCTCACCCTGCCCCCTTCCAACCCCTCAGTTCCCATCCTCC |
| | | AGCAGCTGTTTGTGTGCTGCCTCTGAAGTCCACACTGAACAAACTTCAGCCTACTCATGTCCCTAAAATGGGC |
| | | AAACATTGCAAGCAGCAAACAGCAAACACACAGCCCTCCCTGCCTGCTGACCTTGGAGCTGGGGCAGAGGTC |
| | | AGAGACCTCTCTGGGCCCATGCCACCTCCAACATCCACTCGACCCCTTGGAATTTCGGTGGAGAGGAGCAGA |
| | | GGTTGTCCTGGCGTGGTTTAGGTAGTGTGAGAGGGTCCGGGTTCAAAACCACTTGCTGGGTGGGGAGTCGTCA |
| | | GTAAGTGGCTATGCCCCGACCCCGAAGCCTGTTTCCCCATCTGTACAATGGAAATGATAAAGACGCCCATCTG |
| | | ATAGGGTTTTTGTGGCAAATAAACATTTGGTTTTTTTGTTTTGTTTTGTTTTGTTTTTTGAGATGGAGGTTTGCT |
| | | CTGTCGCCCAGGCTGGAGTGCAGTGACACAATCTCATCTCACCACAACCTTCCCCTGCCTCAGCCTCCCAAGT |
| | | AGCTGGGATTACAAGCATGTGCCACCACACCTGGCTAATTTTCTATTTTTAGTAGAGACGGGTTTCTCCATGTT |
| | | GGTCAGCCTCAGCCTCCCAAGTAACTGGGATTACAGGCCTGTGCCACCACACCCGGCTAATTTTTTCTATTTTT |
| | | GACAGGGACGGGGTTTCACCATGTTGGTCAGGCTGGTCTAGAGGTACCGGATCTTGCTACCAGTGGAACAGC |
| | | CACTAAGGATTCTGCAGTGAGAGCAGAGGGCCAGCTAAGTGGTACTCTCCCAGAGACTGTCTGACTCACGCC |
| | | ACCCCCTCCACCTTGGACACAGGACGCTGTGGTTTCTGAGCCAGGTACAATGACTCCTTTCGGTAAGTGCAGT |
| | | GGAAGCTGTACACTGCCCAGGCAAAGCGTCCGGGCAGCGTAGGCGGGCGACTCAGATCCCAGCCAGTGGACT |
| | | TAGCCCCTGTTTGCTCCTCCGATAACTGGGGTGACCTTGGTTAATATTCACCAGCAGCCTCCCCCGTTGCCCCT |
| | | CTGGATCCACTGCTTAAATACGGACGAGGACAGGGCCCTGTCTCCTCAGCTTCAGGCACCACCACTGACCTGG |
| | | GACAGTGCCGCCACCATGATCATGGCCGAGAGCCCTGGCCTGATCACCATCTGCCTGCTGGGCTACCTGCTGA |
| | | GCGCCGAGTGCACCGTGTTCCTGGACCACGAGAACGCCAACAAGATCCTGAACCGGCCCAAGAGATACAACA |
| | | GCGGCAAGCTGGAGGAGTTCGTGCAGGGCAACCTGGAGAGGGAGTGCATGGAGGAGAAGTGCAGCTTCGAG |
| | | GAGGCCAGGGAAGTGTTCGAGAACACCGAGCGGACCACCGAGTTCTGGAAGCAGTACGTGGACGGCGACCA |
| | | GTGCGAGAGCAACCCTTGCCTGAACGGCGGCAGCTGCAAGGACGACATCAACAGCTACGAGTGCTGGTGCCC |
| | | TTTCGGCTTCGAGGGCAAGAACTGCGAGCTGGACGTGACCTGCAACATCAAGAACGGCCGCTGCGAGCAGTT |
| | | CTGCAAGAACAGCGCCGACAACAAAGTGGTGTGTAGCTGCACCGAGGGCTACAGACTGGCCGAGAACCAGA |
| | | AGAGCTGCGAGCCCGCCGTGCCCTTCCCCTGCGGCAGAGTGAGCGTGTCCCAGACCAGCAAGCTGACCAGAG |
| | | CCGAGACCGTGTTCCCCGACGTGGACTACGTGAATAGCACCGAGGCCGAGACCATCCTGGACAACATCACCC |
| | | AGAGCACCCAGTCCTTCAACGACTTCACCAGAGTTGTGGGCGGCGAGGACGCCAAGCCCGGCCAGTTCCCCT |
| | | GGCAGGTGGTGCTGAACGGCAAAGTGGATGCCTTCTGCGGCGGCAGCATCGTGAACGAGAAGTGGATCGTGA |
| | | CAGCCGCCCACTGCGTGGAGACCGGCGTGAAGATCACCGTGGTGGCCGGCGAACACAATATCGAGGAGACC |
| | | GAGCACACCGAGCAGAAGCGGAACGTCATCCGGATTATCCCCCACCACAACTACAACGCCGCCATCAACAAG |
| | | TACAACCACGACATCGCCCTGCTGGAGCTGGACGAGCCTCTGGTGCTGAATAGCTACGTGACCCCCATCTGCA |
| | | TCGCCGACAAGGAGTACACCAACATCTTCCTGAAGTTCGGCAGCGGCTACGTGTCCGGCTGGGGCAGAGTGT |
| | | TCCACAAGGGCAGAAGCGCCCTGGTGCTGCAGTACCTGAGAGTGCCCCTGGTGGACAGAGCCACCTGCCTGT |
| | | TGAGCACCAAGTTCACCATCTACAACAACATGTTCTGCGCCGGCTTCCACGAGGGCGGCAGAGACAGCTGCC |
| | | AGGGCGACAGCGGCGGACCCCACGTGACCGAAGTGGAGGGCACCAGCTTCCTGACCGGCATCATCAGCTGGG |
| | | GCGAGGAGTGCGCCATGAAGGGCAAGTACGGCATCTACACCAAAGTGAGCCGGTACGTGAACTGGATCAAG |
| | | GAGAAAACCAAGCTGACCTGAGAGCATCTTACCGCCATTTATTCCCATATTTGTTCTGTTTTTCTTGATTTGGG |
| | | TATACATTTAAATGTTAATAAAACAAAATGGTGGGGCAATCATTTACATTTTTAGGGATATGTAATTACTAGT |
| | | TCAGGTGTATTGCCACAAGACAAACATGTTAAGAAACTTTCCCGTTATTTACGCTCTGTTCCTGTTAATCAACC |
| | | TCTGGATTACAAAATTTGTGAAAGATTGACTGATATTCTTAACTATGTTGCTCCTTTTACGCTGTGTGGATATG |
| | | CTGCTTTATAGCCTCTGTATCTAGCTATTGCTTCCCGTACGGCTTTCGTTTTCTCCTCCTTGTATAAATCCTGGT |
| | | TGCTGTCTCTTTTAGAGGAGTTGTGGCCCGTTGTCCGTCAACGTGGCGTGGTGTGCTCTGTGTTTGCTGACGCA |
| | | ACCCCCACTGGCTGGGGCATTGCCACCACCTGTCAACTCCTTTCTGGGACTTTCGCTTTCCCCCTCCCGATCGC |
| | | CACGGCAGAACTCATCGCCGCCTGCCTTGCCCGCTGCTGGACAGGGGCTAGGTTGCTGGGCACTGATAATTCC |
| | | GTGGTGTTGTCTGTGCCTTCTAGTTGCCAGCCATCTGTTGTTTGCCCCTCCCCCGTGCCTTCCTTGACCCTGGA |
| | | AGGTGCCACTCCCACTGTCCTTTCCTAATAAAATGAGGAAATTGCATCGCATTGTCTGAGTAGGTGTCATTCT |
| | | ATTCTGGGGGGTGGGGTGGGGCAGGACAGCAAGGGGGAGGATTGGGAAGACAATAGCAGGCATGCTGGGGA |
| | | TGCGGTGGGCTCTATGGCAGGAACCCCTAGTGATGGAGTTGGCCACTCCCTCTCTGCGCGCTCGCTCGCTCAC |
| | | TGAGGCCGCCCGGGAAACCCGGGCGTGCGCCTCAGTGAGCGAGCGAGCGCGCAGCTGCCTGCAGG |
| SEQ ID NO: | Construct α | CCTGCAGGCAGCTGCGCGCTCGCTCGCTCACTGAGGCCGCCCGGGCAAAGCCCGGGCGTCGGGCGACCTTTG |
| 61 | | GTCGCCCGGCCTCAGTGAGCGAGCGAGCGCGCAGAGAGGGAGTGGCCAACTCCATCACTAGGGGTTCCTTGT |
| | | AGTTAATGATTAACCCGCCATGCTACTTATCTACGTAGCCATGCTCTAGAGCGGCCGCACGCGTAGATCTTCA |
| | | ATATTGGCCATTAGCCATATTATTCATTGGTTATATAGCATAAATCAATATTGGCTATTGGCCATTGCATACGT |
| | | TGTATCTATATCATAATATGTACATTTATATTGGCTCATGTCCAATATGACCGCCATGTTGGCATTGATTATTG |
| | | ACTAGTTATTAATAGTAATCAATTACGGGGTCATTAGTTCATAGCCCATATATGGAGTTCCGCGTTACATAAC |
| | | TTACGGTAAATGGCCCGCCTGGCTGACCGCCCAACGACCCCCGCCCATTGACGTCAATAATGACGTATGTTCC |
| | | CATAGTAACGCCAATAGGGACTTTCCATTGACGTCAATGGGTGGAGTATTTACGGTAAACTGCCCACTTGGCA |
| | | GTACATCAAGTGTATCATATGCCAAGTCCGCCCCCTATTGACGTCAATGACGGTAAATGGCCCGCCTGGCATT |
| | | ATGCCCAGTACATGACCTTACGGGACTTTCCTACTTGGCAGTACATCTACGTATTAGTCATCGCTATTACCATG |
| | | GTCGAGGTGAGCCCCACGTTCTGCTTCACTCTCCCCATCTCCCCCCCCTCCCCACCCCCAATTTTGTATTTATTT |
| | | ATTTTTTAATTATTTTGTGCAGCGATGGGGGCGGGGGGGGGGGGGGGGCGCGCGCCAGGCGGGGCGGGGCGG |
| | | GGCGAGGGGCGGGGCGGGGCGAGGCGGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGAAGCGCGCGGCGGGCGGGAGTCGCTGC |
| | | GACGCTGCCTTCGCCCCGTGCCCCGCTCCGCCGCCGCCTCGCGCCGCCCGCCCCGGCTCTGACTGACCGCGTT |
| | | ACTCCCACAGGTGAGCGGGCGGGACGGCCCTTCTCCTCCGGGCTGTAATTAGCGCTTGGTTTAATGACGGCTT |
| | | GTTTCTTTTCTGTGGCTGCGTGAAAGCCTTGAGGGGCTCCGGGAGGGCCCTTTGTGCGGGGGGGAGCGGCTCG |
| | | GGGGGTGCGTGCGTGTGTGTGTGCGTGGGGAGCGCCGCGTGCGGCCCGCGCTGCCCGGCGGCTGTGAGCGCT |
| | | GCGGGCGCGGCGCGGGGCTTTGTGCGCTCCGCAGTGTGCGCGAGGGGAGCGCGGCCGGGGGCGGTGCCCCGC |
| | | GGTGCGGGGGGGGCTGCGAGGGGAACAAAGGCTGCGTGCGGGGTGTGTGCGTGGGGGGGTGAGCAGGGGGT |
| | | GTGGGCGCGGCGGTCGGGCTGTAACCCCCCCCTGCACCCCCCTCCCCGAGTTGCTGAGCACGGCCCGGCTTCG |
| | | GGTGCGGGGCTCCGTACGGGGCGTGGCGCGGGGCTCGCCGTGCCGGGCGGGGGGTGGCGGCAGGTGGGGGT |
| | | GCCGGGCGGGGCGGGGCCGCCTCGGGCCGGGGAGGGCTCGGGGGAGGGGCGCGGCGGCCCCCGGAGCGCCG |
| | | GCGGCTGTCGAGGCGCGGCGAGCCGCAGCCATTGCCTTTTATGGTAATCGTGCGAGAGGGCGCAGGGACTTC |
| | | CTTTGTCCCAAATCTGTGCGGAGCCGAAATCTGGGAGGCGCCGCCGCACCCCCTCTAGCGGGCGCGGGGCGA |
| | | AGCGGTGCGGCGCCGGCAGGAAGGAAATGGGCGGGGAGGGCCTTCGTGCGTCGCCGCGCCGCCGTCCCCTTC |
| | | TCCCTCTCCAGCCTCGGGGCTGTCCGCGGGGGGACGGCTGCCTTCGGGGGGGACGGGGCAGGGCGGGGTTCG |
| | | GCTTCTGGCGTGTGACCGGCGGCTCTAGAGCCTCTGCTAACCATGTTTTAGCCTTCTTCTTTTTCCTACAGCTC |
| | | CTGGGCAACGTGCTGGTTATTGTGCTGTCTCATCATTTGTCGACAGAATTCCTCGAAGATCCGAAGGGGTTCA |
| | | AGCTTGGCATTCCGGTACTGTTGGTAAAGCCACCATGGAAGACGCCAAAAACATAAAGAAAGGCCCGGCGCC |
| | | ATTCTATCCGCTGGAAGATGGAACCGCTGGAGAGCAACTGCATAAGGCTATGAAGAGATACGCCCTGGTTCC |
| | | TGGAACAATTGCTTTTACAGATGCACATATCGAGGTGGACATCACTTACGCTGAGTACTTCGAAATGTCCGTT |
| | | CGGTTGGCAGAAGCTATGAAACGATATGGGCTGAATACAAATCACAGAATCGTCGTATGCAGTGAAAACTCT |
| | | CTTCAATTCTTTATGCCGGTGTTGGGCGCGTTATTTATCGGAGTTGCAGTTGCGCCCGCGAACGACATTTATAA |
| | | TGAACGTGAATTGCTCAACAGTATGGGCATTTCGCAGCCTACCGTGGTGTTCGTTTCCAAAAAGGGGTTGCAA |
| | | AAAATTTTGAACGTGCAAAAAAAGCTCCCAATCATCCAAAAAATTATTATCATGGATTCTAAAACGGATTACC |
| | | AGGGATTTCAGTCGATGTACACGTTCGTCACATCTCATCTACCTCCCGGTTTTAATGAATACGATTTTGTGCCA |
| | | GAGTCCTTCGATAGGGACAAGACAATTGCACTGATCATGAACTCCTCTGGATCTACTGGTCTGCCTAAAGGTG |
| | | TCGCTCTGCCTCATAGAACTGCCTGCGTGAGATTCTCGCATGCCAGAGATCCTATTTTTGGCAATCAAATCATT |
| | | CCGGATACTGCGATTTTAAGTGTTGTTCCATTCCATCACGGTTTTGGAATGTTTACTACACTCGGATATTTGAT |
| | | ATGTGGATTTCGAGTCGTCTTAATGTATAGATTTGAAGAAGAGCTGTTTCTGAGGAGCCTTCAGGATTACAAG |
| | | ATTCAAAGTGCGCTGCTGGTGCCAACCCTATTCTCCTTCTTCGCCAAAAGCACTCTGATTGACAAATACGATTT |
| | | ATCTAATTTACACGAAATTGCTTCTGGTGGCGCTCCCCTCTCTAAGGAAGTCGGGGAAGCGGTTGCCAAGAGG |
| | | TTCCATCTGCCAGGTATCAGGCAAGGATATGGGCTCACTGAGACTACATCAGCTATTCTGATTACACCCGAGG |
| | | GGGATGATAAACCGGGCGCGGTCGGTAAAGTTGTTCCATTTTTTGAAGCGAAGGTTGTGGATCTGGATACCGG |
| | | GAAAACGCTGGGCGTTAATCAAAGAGGCGAACTGTGTGTGAGAGGTCCTATGATTATGTCCGGTTATGTAAA |
| | | CAATCCGGAAGCGACCAACGCCTTGATTGACAAGGATGGATGGCTACATTCTGGAGACATAGCTTACTGGGA |
| | | CGAAGACGAACACTTCTTCATCGTTGACCGCCTGAAGTCTCTGATTAAGTACAAAGGCTATCAGGTGGCTCCC |
| | | GCTGAATTGGAATCCATCTTGCTCCAACACCCCAACATCTTCGACGCAGGTGTCGCAGGTCTTCCCGACGATG |
| | | ACGCCGGTGAACTTCCCGCCGCCGTTGTTGTTTTGGAGCACGGAAAGACGATGACGGAAAAAGAGATCGTGG |
| | | ATTACGTCGCCAGTCAAGTAACAACCGCGAAAAAGTTGCGCGGAGGAGTTGTGTTTGTGGACGAAGTACCGA |
| | | AAGGTCTTACCGGAAAACTCGACGCAAGAAAAATCAGAGAGATCCTCATAAAGGCCAAGAAGGGCGGAAAG |
| | | ATCGCCGTGTAATTCTAGAGTCGGGGCGGCCGGCCGCTTCGAGCAGACATGATAAGATACATTGATGAGTTT |
| | | GGACAAACCACAACTAGAATGCAGTGAAAAAAATGCTTTATTTGTGAAATTTGTGATGCTATTGCTTTATTTG |
| | | TAACCATTATAAGCTGCAATAAACAAGTTAACAACAACAATTGCATTCATTTTATGTTTCAGGTTCAGGGGGA |
| | | GGTGTGGGAGGTTTTTTAAAGCAAGTAAAACCTCTACAAATGTGGTAAAATCGATAAGTGCGGACCGAGCGG |
| | | CCGCTCTAGAGCATGGCTACGTAGATAAGTAGCATGGCGGGTTAATCATTAACTACACCTGCAGGCAGCTGC |
| | | GCGCTCGCTCGCTCACTGAGGCCGCCCGGGCGTCGGGCGACCTTTGGTCGCCCGGCCTCAGTGAGCGAGCGA |
| | | GCGCGCAGAGAGGGAGTGGCCAACTCCATCACTAGGGGTTCCT |
| SEQ ID NO: | Construct β | CCTGCAGGCAGCTGCGCGCTCGCTCGCTCACTGAGGCCGCCCGGGCAAAGCCCGGGCGTCGGGCGACCTTTG |
| 62 | | GTCGCCCGGCCTCAGTGAGCGAGCGAGCGCGCAGAGAGGGAGTGGCCAACTCCATCACTAGGGGTTCCTTGT |
| | | AGTTAATGATTAACCCGCCATGCTACTTATCTACGTAGCCATGCTCTAGAGCGGCCGCGCTAGCCCCTAAAAT |
| | | GGGCAAACATTGCAAGCAGCAAACAGCAAACACACAGCCCTCCCTGCCTGCTGACCTTGGAGCTGGGGCAGA |
| | | GGTCAGAGACCTCTCTGGGCCCATGCCACCTCCAACATCCACTCGACCCCTTGGAATTTCGGTGGAGAGGAGC |
| | | AGAGGTTGTCCTGGCGTGGTTTAGGTAGTGTGAGAGGGGAATGACTCCTTTCGGTAAGTGCAGTGGAAGCTGT |
| | | ACACTGCCCAGGCAAAGCGTCCGGGCAGCGTAGGCGGGCGACTCAGATCCCAGCCAGTGGACTTAGCCCCTG |
| | | TTTGCTCCTCCGATAACTGGGGTGACCTTGGTTAATATTCACCAGCAGCCTCCCCCGTTGCCCCTCTGGATCCA |
| | | CTGCTTAAATACGGACGAGGACACTCGAGGGCCCTGTCTCCTCAGCTTCAGGCACCACCACTGACCTGGGAC |
| | | AGTGAATCCGGACATCGATTCTAAGGTAAATATAAAATTTTTAAGTGTATAATTTGTTAAACTACTGATTCTA |
| | | ATTGTTTCTCTCTTTTAGATTCCAACCTTTGGAACTGAATTCTAGACCACCATGCAGAGGGTGAACATGATCAT |
| | | GGCTGAGAGCCCTGGCCTGATCACCATCTGCCTGCTGGGCTACCTGCTGTCTGCTGAGTGCACTGTGTTCCTG |
| | | GACCATGAGAATGCCAACAAGATCCTGAACAGGCCCAAGAGATACAACTCTGGCAAGCTGGAGGAGTTTGTG |
| | | CAGGGCAACCTGGAGAGGGAGTGCATGGAGGAGAAGTGCAGCTTTGAGGAGGCCAGGGAGGTGTTTGAGAA |
| | | CACTGAGAGGACCACTGAGTTCTGGAAGCAGTATGTGGATGGGGACCAGTGTGAGAGCAACCCCTGCCTGAA |
| | | TGGGGGCAGCTGCAAGGATGACATCAACAGCTATGAGTGCTGGTGCCCCTTTGGCTTTGAGGGCAAGAACTG |
| | | TGAGCTGGATGTGACCTGCAACATCAAGAATGGCAGATGTGAGCAGTTCTGCAAGAACTCTGCTGACAACAA |
| | | GGTGGTGTGCAGCTGCACTGAGGGCTACAGGCTGGCTGAGAACCAGAAGAGCTGTGAGCCTGCTGTGCCATT |
| | | CCCATGTGGCAGAGTGTCTGTGAGCCAGACCAGCAAGCTGACCAGGGCTGAGGCTGTGTTCCCTGATGTGGA |
| | | CTATGTGAACAGCACTGAGGCTGAAACCATCCTGGACAACATCACCCAGAGCACCCAGAGCTTCAATGACTT |
| | | CACCAGGGTGGTGGGGGGGGAGGATGCCAAGCCTGGCCAGTTCCCCTGGCAAGTGGTGCTGAATGGCAAGGT |
| | | GGATGCCTTCTGTGGGGGCAGCATTGTGAATGAGAAGTGGATTGTGACTGCTGCCCACTGTGTGGAGACTGG |
| | | GGTGAAGATCACTGTGGTGGCTGGGGAGCACAACATTGAGGAGACTGAGCACACTGAGCAGAAGAGGAATG |
| | | TGATCAGGATCATCCCCCACCACAACTACAATGCTGCCATCAACAAGTACAACCATGACATTGCCCTGCTGGA |
| | | GCTGGATGAGCCCCTGGTGCTGAACAGCTATGTGACCCCCATCTGCATTGCTGACAAGGAGTACACCAACATC |
| | | TTCCTGAAGTTTGGCTCTGGCTATGTGTCTGGCTGGGGCAGGGTGTTCCACAAGGGCAGGTCTGCCCTGGTGC |
| | | TGCAGTACCTGAGGGTGCCCCTGGTGGACAGGGCCACCTGCCTGAGGAGCACCAAGTTCACCATCTACAACA |
| | | ACATGTTCTGTGCTGGCTTCCATGAGGGGGGCAGGGACAGCTGCCAGGGGGACTCTGGGGGCCCCCATGTGA |
| | | CTGAGGTGGAGGGCACCAGCTTCCTGACTGGCATCATCAGCTGGGGGGAGGAGTGTGCCATGAAGGGCAAGT |
| | | ATGGCATCTACACCAAAGTCTCCAGATATGTGAACTGGATCAAGGAGAAGACCAAGCTGACCTAATGACTCC |
| | | ATGGTTCGAATGCTTTATTTGTGAAATTTGTGATGCTATTGCTTTATTTGTAACCATTATAAGCTGCAATAAAC |
| | | AAGTTAACAACAACAATTGCATTCATTTTATGTTTCAGGTTCAGGGGGAGGTGTGGGAGGTTTTTTAAAACTA |
| | | GTGCGGCCGCTCTAGAGCATGGCTACGTAGATAAGTAGCATGGCGGGTTAATCATTAACTACAAGGAACCCC |
| | | TAGTGATGGAGTTGGCCACTCCCTCTCTGCGCGCTCGCTCGCTCACTGAGGCCGGGCGACCAAAGGTCGCCCG |
| | | ACGCCCGGGCGGCCTCAGTGAGCGAGCGAGCGCGCAGCTGCCTGCAGG |
| SEQ ID NO: | Mut2-L | CCTGCAGGCAGCTGCGCGCTCGCTCGCTCACTGAGGCCGCCCGGGAAACCCGGGCGTGCCCGGGCGCCTCAG |
| 63 | | TGAGCGAGCGAGCGCGCAGAGAGGGAGTGGCCAACTCCATCACTAGGGGTTCCT |
| SEQ ID NO: | Mut3-R | AGGAACCCCTAGTGATGGAGTTGGCCACTCCCTCTCTGCGCGCTCGCTCGCTCACTGAGGCCGCCCGGGAAAC |
| 64 | | CCGGGCGTGCGCCTCAGTGAGCGAGCGAGCGCGCAGCTGCCTGCAGG |
| SEQ ID NO: | Selected portion | GCGCGCTCGCTCGCTCACTGAGGCCGCCCGGGCGTCGGGCGACCTTTGGTCGCCCGGCCTCAGTGAGCGAGCGAGCGCGC |
| 65 | of SEQ ID | |
| | NO: 52 | |
| | Containing RBE | |
| SEQ ID NO: | IE1 promoter | AATAAACGATAACGCCGTTGGTGGCGTGAGGCATGTAAAAGGTTACATCATTATCTTGTTCGCCATCCGGTTG |
| 66 | fragment | GTATAAATAGACGTTCATGTTGGTTTTTGTTTCAGTTGCAAGTTGGCTGCGGCGCGCGCAGCACCTTT |
| SEQ ID NO: | Rep 78 | CGCAGCCACC- |
| 67 | nucleotide | ATGGCGGGGTTTTACGAGATTGTGATTAAGGTCCCCAGCGACCTTGACGAGCATCTGCCCGGCATTTCTGACA |
| | sequence (incl. | GCTTTGTGAACTGGGTGGCCGAGAAGGAATGGGAGTTGCCGCCAGATTCTGACATGGATCTGAATCTGATTG |
| | Kozak seq. | AGCAGGCACCCCTGACCGTGGCCGAGAAGCTGCAGCGCGACTTTCTGACGGAATGGCGCCGTGTGAGTAAGG |
| | underlined) | CCCCGGAGGCCCTTTTCTTTGTGCAATTTGAGAAGGGAGAGAGCTACTTCCACATGCACGTGCTCGTGGAAAC |
| | | CACCGGGGTGAAATCCATGGTTTTGGGACGTTTCCTGAGTCAGATTCGCGAAAAACTGATTCAGAGAATTTAC |
| | | CGCGGGATCGAGCCGACTTTGCCAAACTGGTTCGCGGTCACAAAGACCAGAAATGGCGCCGGAGGCGGGAA |
| | | CAAGGTGGTGGATGAGTGCTACATCCCCAATTACTTGCTCCCCAAAACCCAGCCTGAGCTCCAGTGGGCGTGG |
| | | ACTAATATGGAACAGTATTTAAGCGCCTGTTTGAATCTCACGGAGCGTAAACGGTTGGTGGCGCAGCATCTGA |
| | | CGCACGTGTCGCAGACGCAGGAGCAGAACAAAGAGAATCAGAATCCCAATTCTGATGCGCCGGTGATCAGAT |
| | | CAAAAACTTCAGCCAGGTACATGGAGCTGGTCGGGTGGCTCGTGGACAAGGGGATTACCTCGGAGAAGCAGT |
| | | GGATCCAGGAGGACCAGGCCTCATACATCTCCTTCAATGCGGCCTCCAACTCGCGGTCCCAAATCAAGGCTGC |
| | | CTTGGACAATGCGGGAAAGATTATGAGCCTGACTAAAACCGCCCCCGACTACCTGGTGGGCCAGCAGCCCGT |
| | | GGAGGACATTTCCAGCAATCGGATTTATAAAATTTTGGAACTAAACGGGTACGATCCCCAATATGCGGCTTCC |
| | | GTCTTTCTGGGATGGGCCACGAAAAAGTTCGGCAAGAGGAACACCATCTGGCTGTTTGGGCCTGCAACTACC |
| | | GGGAAGACCAACATCGCGGAGGCCATAGCCCACACTGTGCCCTTCTACGGGTGCGTAAACTGGACCAATGAG |
| | | AACTTTCCCTTCAACGACTGTGTCGACAAGATGGTGATCTGGTGGGAGGAGGGGAAGATGACCGCCAAGGTC |
| | | GTGGAGTCGGCCAAAGCCATTCTCGGAGGAAGCAAGGTGCGCGTGGACCAGAAATGCAAGTCCTCGGCCCAG |
| | | ATAGACCCGACTCCCGTGATCGTCACCTCCAACACCAACATGTGCGCCGTGATTGACGGGAACTCAACGACCT |
| | | TCGAACACCAGCAGCCGTTGCAAGACCGGATGTTCAAATTTGAACTCACCCGCCGTCTGGATCATGACTTTGG |
| | | GAAGGTCACCAAGCAGGAAGTCAAAGACTTTTTCCGGTGGGCAAAGGATCACGTGGTTGAGGTGGAGCATGA |
| | | ATTCTACGTCAAAAAGGGTGGAGCCAAGAAAAGACCCGCCCCCAGTGACGCAGATATAAGTGAGCCCAAAC |
| | | GGGTGCGCGAGTCAGTTGCGCAGCCATCGACGTCAGACGCGGAAGCTTCGATCAACTACGCAGACAGGTACC |
| | | AAAACAAATGTTCTCGTCACGTGGGCATGAATCTGATGCTGTTTCCCTGCAGACAATGCGAGAGAATGAATC |
| | | AGAATTCAAATATCTGCTTCACTCACGGACAGAAAGACTGTTTAGAGTGCTTTCCCGTGTCAGAATCTCAACC |
| | | CGTTTCTGTCGTCAAAAAGGCGTATCAGAAACTGTGCTACATTCATCATATCATGGGAAAGGTGCCAGACGCT |
| | | TGCACTGCCTGCGATCTGGTCAATGTGGATTTGGATGACTGCATCTTTGAACAATAA |
| SEQ ID NO: | Polyhedron | ATCATGGAGATAATTAAAATGATAACCATCTCGCAAATAAATAAGTATTTTACTGTTTTCGTAACAGTTTTGT |
| 68 | promoter | AATAAAAAAACCTATAAATATTCCGGATTATTCATACCGTCCCACCATCGGGCGCG |
| | sequence | |
| SEQ ID NO: | Rep58 DNA | GCCGCCACC- |
| 69 | sequence | ATGGAGTTGGTGGGCTGGCTCGTGGACAAAGGCATTACTTCGGAAAAGCAGTGGATTCAGGAGGATCAGGCATCTTACATCTCATTCA |
| | | ACGCTGCCAGTAACTCGAGGTCCCAGATCAAGGCAGCGCTGGACAACGCGGGAAAGATTATGAGTCTGACCAAAACTGCTCCAGACTA |
| | | CCTCGTTGGTCAGCAACCGGTGGAAGATATCTCCAGCAACAGGATCTACAAGATTCTGGAGCTCAACGGCTACGACCCTCAATACGCTG |
| | | CCTCAGTGTTCTTGGGTTGGGCCACCAAGAAATTCGGCAAGAGAAACACTATCTGGCTGTTCGGCCCCGCTACCACTGGAAAGACAAAC |
| | | ATCGCAGAAGCGATGCTCACACGGTGCCATTCTACGGCTGCGTCAACTGGACAAACGAGAACTTCCCGTTCAACGACTGTGTCGATAA |
| | | GATGGTTATCTGGTGGGAGGAAGGAAAGATGACGGCCAAAGTGGTCGAAAGCGCCAAGGCAATTCTGGGTGGCTCTAAAGTGCGCGT |
| | | CGACCAGAAGTGCAAATCTTCAGCTCAAATCGATCCTACCCCCGTTATTGTGACATCAAACACGAACATGTGTGCCGTGATCGACGGAA |
| | | ACAGTACAACGTTCGAACACCAGCAACCTCTCCAGGATCGTATGTTCAAGTTCGAGCTCACCCGCCGTTTGGACCATGATTTCGGCAAG |
| | | GTCACTAAACAAGAGGTTAAGGACTTCTTCCGCTGGGCTAAAGATCACGTTGTGGAGGTTGAACATGAGTTCTACGTCAAGAAAGGAG |
| | | GTGCTAAGAAACGTCCAGCCCCGTCGGACGCAGATATCTCCGAACCTAAGAGGGTGAGAGAGTCGGTCGCACAGCCAAGCACTTCTGA |
| | | CGCAGAAGCTTCCATTAACTACGCAGATAGGTACCAAAACAAGTGCAGCAGACACGTGGGTATGAACTTGATGCTGTTCCCATGCCGCC |
| | | AGTGTGAGCGTATGAACCAAAACTCTAACATCTGTTTCACACATGGCCAGAAGGACTGCCTCGAATGTTTCCCTGTGTCAGAGAGTCAG |
| | | CCCGTCTCAGTCGTTAAGAAAGCTTACCAAAAGTTGTGCTACATCCACCATATTATGGGTAAAGTCCCTGATGCCTGTACCGCTTGTGAT |
| | | CTGGTCAACGTGGATTTGGACGACTGTATTTTCGAGCAATAA |
| SEQ ID NO: | MND Promoter | GAACAGAGAAACAGGAGAATATGGGCCAAACAGGATATCTGTGGTAAGCAGTTCCTGCCCCGGCTCAGGGCC |
| 70 | | AAGAACAGTTGGAACAGCAGAATATGGGCCAAACAGGATATCTGTGGTAAGCAGTTCCTGCCCCGGCTCAGG |
| | | GCCAAGAACAGATGGTCCCCAGATGCGGTCCCGCCCTCAGCAGTTTCTAGAGAACCATCAGATGTTTCCAGG |
| | | GTGCCCCAAGGACCTGAAATGACCCTGTGCCTTATTTGAACTAACCAATCAGTTCGCTTCTCGCTTCTGTTCGC |
| | | GCGCTTCTGCTCCCCGAGCTCTATATAAGCAGAGCTCGTTTAGTGAACCGTCAGATCGCCTGGAGACGCCATC |
| | | CACGCTGTTTTGACTTCCATAGAAG |
| SEQ ID NO: | Luciferase (with | GCCGCCACC- |
| 71 | Kozak Seq | ATGGAAGACGCCAAAAACATAAAGAAAGGCCCGGCGCCATTCTATCCGCTGGAAGATGGAACCGCTGGAGA |
| | underlined | GCAACTGCATAAGGCTATGAAGAGATACGCCCTGGTTCCTGGAACAATTGCTTTTACAGATGCACATATCGAG |
| | | GTGGACATCACTTACGCTGAGTACTTCGAAATGTCCGTTCGGTTGGCAGAAGCTATGAAACGATATGGGCTGA |
| | | ATACAAATCACAGAATCGTCGTATGCAGTGAAAACTCTCTTCAATTCTTTATGCCGGTGTTGGGCGCGTTATTT |
| | | ATCGGAGTTGCAGTTGCGCCCGCGAACGACATTTATAATGAACGTGAATTGCTCAACAGTATGGGCATTTCGC |
| | | AGCCTACCGTGGTGTTCGTTTCCAAAAAGGGGTTGCAAAAAATTTTGAACGTGCAAAAAAAGCTCCCAATCA |
| | | TCCAAAAAATTATTATCATGGATTCTAAAACGGATTACCAGGGATTTCAGTCGATGTACACGTTCGTCACATC |
| | | TCATCTACCTCCCGGTTTTAATGAATACGATTTTGTGCCAGAGTCCTTCGATAGGGACAAGACAATTGCACTG |
| | | ATCATGAACTCCTCTGGATCTACTGGTCTGCCTAAAGGTGTCGCTCTGCCTCATAGAACTGCCTGCGTGAGAT |
| | | TCTCGCATGCCAGAGATCCTATTTTTGGCAATCAAATCATTCCGGATACTGCGATTTTAAGTGTTGTTCCATTC |
| | | CATCACGGTTTTGGAATGTTTACTACACTCGGATATTTGATATGTGGATTTCGAGTCGTCTTAATGTATAGATT |
| | | TGAAGAAGAGCTGTTTCTGAGGAGCCTTCAGGATTACAAGATTCAAAGTGCGCTGCTGGTGCCAACCCTATTC |
| | | TCCTTCTTCGCCAAAAGCACTCTGATTGACAAATACGATTTATCTAATTTACACGAAATTGCTTCTGGTGGCGC |
| | | TCCCCTCTCTAAGGAAGTCGGGGAAGCGGTTGCCAAGAGGTTCCATCTGCCAGGTATCAGGCAAGGATATGG |
| | | GCTCACTGAGACTACATCAGCTATTCTGATTACACCCGAGGGGGATGATAAACCGGGCGCGGTCGGTAAAGT |
| | | TGTTCCATTTTTTGAAGCGAAGGTTGTGGATCTGGATACCGGGAAAACGCTGGGCGTTAATCAAAGAGGCGA |
| | | ACTGTGTGTGAGAGGTCCTATGATTATGTCCGGTTATGTAAACAATCCGGAAGCGACCAACGCCTTGATTGAC |
| | | AAGGATGGATGGCTACATTCTGGAGACATAGCTTACTGGGACGAAGACGAACACTTCTTCATCGTTGACCGC |
| | | CTGAAGTCTCTGATTAAGTACAAAGGCTATCAGGTGGCTCCCGCTGAATTGGAATCCATCTTGCTCCAACACC |
| | | CCAACATCTTCGACGCAGGTGTCGCAGGTCTTCCCGACGATGACGCCGGTGAACTTCCCGCCGCCGTTGTTGT |
| | | TTTGGAGCACGGAAAGACGATGACGGAAAAAGAGATCGTGGATTACGTCGCCAGTCAAGTAACAACCGCGA |
| | | AAAAGTTGCGCGGAGGAGTTGTGTTTGTGGACGAAGTACCGAAAGGTCTTACCGGAAAACTCGACGCAAGAA |
| | | AAATCAGAGAGATCCTCATAAAGGCCAAGAAGGGCGGAAAGATCGCCGTGTAA |
| SEQ ID NO: | WPRE | GAGCATCTTACCGCCATTTATTCCCATATTTGTTCTGTTTTTCTTGATTTGGGTATACATTTAAATGTTAATAAA |
| 72 | | ACAAAATGGTGGGGCAATCATTTACATTTTTAGGGATATGTAATTACTAGTTCAGGTGTATTGCCACAAGACA |
| | | AACATGTTAAGAAACTTTCCCGTTATTTACGCTCTGTTCCTGTTAATCAACCTCTGGATTACAAAATTTGTGAA |
| | | AGATTGACTGATATTCTTAACTATGTTGCTCCTTTTACGCTGTGTGGATATGCTGCTTTATAGCCTCTGTATCTA |
| | | GCTATTGCTTCCCGTACGGCTTTCGTTTTCTCCTCCTTGTATAAATCCTGGTTGCTGTCTCTTTTAGAGGAGTTG |
| | | TGGCCCGTTGTCCGTCAACGTGGCGTGGTGTGCTCTGTGTTTGCTGACGCAACCCCCACTGGCTGGGGCATTG |
| | | CCACCACCTGTCAACTCCTTTCTGGGACTTTCGCTTTCCCCCTCCCGATCGCCACGGCAGAACTCATCGCCGCC |
| | | TGCCTTGCCCGCTGCTGGACAGGGGCTAGGTTGCTGGGCACTGATAATTCCGTGGTGTTGTC |
| SEQ ID NO: | BGH-PolyA | TGTGCCTTCTAGTTGCCAGCCATCTGTTGTTTGCCCCTCCCCCGTGCCTTCCTTGACCCTGGAAGGTGCCACTC |
| 73 | | CCACTGTCCTTTCCTAATAAAATGAGGAAATTGCATCGCATTGTCTGAGTAGGTGTCATTCTATTCTGGGGGGT |
| | | GGGGTGGGGCAGGACAGCAAGGGGGAGGATTGGGAAGACAATAGCAGGCATGCTGGGGATGCGGTGGGCTC |
| | | TATGGC |
| SEQ ID NO: | HLCR-AAT | GGCTCAGAGGCTCAGAGGCACACAGGAGTTTCTGGGCTCACCCTGCCCCCTTCCAACCCCTCAGTTCCCATCC |
| 74 | promoter | TCCAGCAGCTGTTTGTGTGCTGCCTCTGAAGTCCACACTGAACAAACTTCAGCCTACTCATGTCCCTAAAATG |
| | | GGCAAACATTGCAAGCAGCAAACAGCAAACACACAGCCCTCCCTGCCTGCTGACCTTGGAGCTGGGGCAGAG |
| | | GTCAGAGACCTCTCTGGGCCCATGCCACCTCCAACATCCACTCGACCCCTTGGAATTTCGGTGGAGAGGAGCA |
| | | GAGGTTGTCCTGGCGTGGTTTAGGTAGTGTGAGAGGGTCCGGGTTCAAAACCACTTGCTGGGTGGGGAGTCGT |
| | | CAGTAAGTGGCTATGCCCCGACCCCGAAGCCTGTTTCCCCATCTGTACAATGGAAATGATAAAGACGCCCATC |
| | | TGATAGGGTTTTTGTGGCAAATAAACATTTGGTTTTTTTGTTTTGTTTTGTTTTGTTTTTTGAGATGGAGGTTTG |
| | | CTCTGTCGCCCAGGCTGGAGTGCAGTGACACAATCTCATCTCACCACAACCTTCCCCTGCCTCAGCCTCCCAA |
| | | GTAGCTGGGATTACAAGCATGTGCCACCACACCTGGCTAATTTTCTATTTTTAGTAGAGACGGGTTTCTCCAT |
| | | GTTGGTCAGCCTCAGCCTCCCAAGTAACTGGGATTACAGGCCTGTGCCACCACACCCGGCTAATTTTTTCTATT |
| | | TTTGACAGGGACGGGGTTTCACCATGTTGGTCAGGCTGGTCTAGAGGTACCGGATCTTGCTACCAGTGGAACA |
| | | GCCACTAAGGATTCTGCAGTGAGAGCAGAGGGCCAGCTAAGTGGTACTCTCCCAGAGACTGTCTGACTCACG |
| | | CCACCCCCTCCACCTTGGACACAGGACGCTGTGGTTTCTGAGCCAGGTACAATGACTCCTTTCGGTAAGTGCA |
| | | GTGGAAGCTGTACACTGCCCAGGCAAAGCGTCCGGGCAGCGTAGGCGGGCGACTCAGATCCCAGCCAGTGGA |
| | | CTTAGCCCCTGTTTGCTCCTCCGATAACTGGGGTGACCTTGGTTAATATTCACCAGCAGCCTCCCCCGTTGCCC |
| | | CTCTGGATCCACTGCTTAAATACGGACGAGGACAGGGCCCTGTCTCCTCAGCTTCAGGCACCACCACTGACCT |
| | | GGGACAGT |
| SEQ ID NO: | | |
| 75-100 | | |
| SEQ ID NO: | Left ITR-2 | GCGCGCTCGCTCGCTCACTGAGGCCGCCCGGGAAACCCGGGCGTGCGCCTCAGTGAGCGAGCGAGCGCGC |
| 101 | | |
| SEQ ID NO: | Right ITR-2 | GCGCGCTCGCTCGCTCACTGAGGCGCACGCCCGGGTTTCCCGGGCGGCCTCAGTGAGCGAGCGAGCGCGC |
| 102 | | |
| SEQ ID NO: | Left ITR-3 | GCGCGCTCGCTCGCTCACTGAGGCCGTCGGGCGACCTTTGGTCGCCCGGCCTCAGTGAGCGAGCGAGCGCGC |
| 103 | | |
| SEQ ID NO: | Right ITR-3 | GCGCGCTCGCTCGCTCACTGAGGCCGGGCGACCAAAGGTCGCCCGACGGCCTCAGTGAGCGAGCGAGCGCGC |
| 104 | | |
| SEQ ID NO: | Left ITR-4 | GCGCGCTCGCTCGCTCACTGAGGCCGCCCGGGCAAAGCCCGGGCGTCGGCCTCAGTGAGCGAGCGAGCGCGC |
| 105 | | |
| SEQ ID NO: | Right ITR-4 | GCGCGCTCGCTCGCTCACTGAGGCCGACGCCCGGGCTTTGCCCGGGCGGCCTCAGTGAGCGAGCGAGCGCGC |
| 106 | | |
| SEQ ID NO: | Left ITR-10 | GCGCGCTCGCTCGCTCACTGAGGCCGCCCGGGCAAAGCCCGGGCGTCGGGCTTTGCCCGGCCTCAGTGAGCG |
| 107 | | AGCGAGCGCGC |
| SEQ ID NO: | Right ITR-10 | GCGCGCTCGCTCGCTCACTGAGGCCGGGCAAAGCCCGACGCCCGGGCTTTGCCCGGGCGGCCTCAGTGAGCG |
| 108 | | AGCGAGCGCGC |
| SEQ ID NO: | Left ITR-17 | GCGCGCTCGCTCGCTCACTGAGGCCGAAACGTCGGGCGACCTTTGGTCGCCCGGCCTCAGTGAGCGAGCGAG |
| 109 | | CGCGC |
| SEQ ID NO: | Right ITR-17 | GCGCGCTCGCTCGCTCACTGAGGCCGGGCGACCAAAGGTCGCCCGACGTTTCGGCCTCAGTGAGCGAGCGAG |
| 110 | | CGCGC |
| SEQ ID NO: | Left ITR-6 | GCGCGCTCGCTCGCTCACTGAGGCAAAGCCTCAGTGAGCGAGCGAGCGCGC |
| 111 | | |
| SEQ ID NO: | Right ITR-6 | GCGCGCTCGCTCGCTCACTGAGGCTTTGCCTCAGTGAGCGAGCGAGCGCGC |
| 112 | | |
| SEQ ID NO: | Left ITR-1 | GCGCGCTCGCTCGCTCACTGAGGCCGCCCGGGCGTCGGGCGACCTTTGGTCGCCCGGCCTCAGTGAGCGAGC |
| 113 | | GAGCGCGC |
| SEQ ID NO: | Right ITR-1 | GCGCGCTCGCTCGCTCACTGAGGCCGGGCGACCAAAGGTCGCCCGACGCCCGGGCGGCCTCAGTGAGCGAGC |
| 114 | | GAGCGCGC |
| SEQ ID NO: | Left ITR-5 | GCGCGCTCGCTCGCTCACTGAGGCGCCCGGGCGTCGGGCGACCTTTGGTCGCCCGGCCTCAGTGAGCGAGCG |
| 115 | | AGCGCGC |
| SEQ ID NO: | Right ITR-5 | GCGCGCTCGCTCGCTCACTGAGGCCGGGCGACCAAAGGTCGCCCGACGCCCGGGCGCCTCAGTGAGCGAGCG |
| 116 | | AGCGCGC |
| SEQ ID NO: | Left ITR-7 | GCGCGCTCGCTCGCTCACTGAGGCCGCCCGGGCAAAGCCCGGGCGTCGGGCGACTTTGTCGCCCGGCCTCAG |
| 117 | | TGAGCGAGCGAGCGCGC |
| SEQ ID NO: | Right ITR-7 | GCGCGCTCGCTCGCTCACTGAGGCCGGGCGACAAAGTCGCCCGACGCCCGGGCTTTGCCCGGGCGGCCTCAG |
| 118 | | TGAGCGAGCGAGCGCGC |
| SEQ ID NO: | Left ITR-8 | GCGCGCTCGCTCGCTCACTGAGGCCGCCCGGGCAAAGCCCGGGCGTCGGGCGATTTTCGCCCGGCCTCAGTG |
| 119 | | AGCGAGCGAGCGCGC |
| SEQ ID NO: | Right ITR-8 | GCGCGCTCGCTCGCTCACTGAGGCCGGGCGAAAATCGCCCGACGCCCGGGCTTTGCCCGGGCGGCCTCAGTG |
| 120 | | AGCGAGCGAGCGCGC |
| SEQ ID NO: | Left ITR-9 | GCGCGCTCGCTCGCTCACTGAGGCCGCCCGGGCAAAGCCCGGGCGTCGGGCGTTTCGCCCGGCCTCAGTGAG |
| 121 | | CGAGCGAGCGCGC |
| SEQ ID NO: | Right ITR-9 | GCGCGCTCGCTCGCTCACTGAGGCCGGGCGAAACGCCCGACGCCCGGGCTTTGCCCGGGCGGCCTCAGTGAG |
| 122 | | CGAGCGAGCGCGC |
| SEQ ID NO: | Left ITR-11 | GCGCGCTCGCTCGCTCACTGAGGCCGCCCGGGAAACCCGGGCGTCGGGCGACCTTTGGTCGCCCGGCCTCAG |
| 123 | | TGAGCGAGCGAGCGCGC |
| SEQ ID NO: | Right ITR-11 | GCGCGCTCGCTCGCTCACTGAGGCCGGGCGACCAAAGGTCGCCCGACGCCCGGGTTTCCCGGGCGGCCTCAG |
| 124 | | TGAGCGAGCGAGCGCGC |
| SEQ ID NO: | Left ITR-12 | GCGCGCTCGCTCGCTCACTGAGGCCGCCCGGAAACCGGGCGTCGGGCGACCTTTGGTCGCCCGGCCTCAGTG |
| 125 | | AGCGAGCGAGCGCGC |
| SEQ ID NO: | Right ITR-12 | GCGCGCTCGCTCGCTCACTGAGGCCGGGCGACCAAAGGTCGCCCGACGCCCGGTTTCCGGGCGGCCTCAGTG |
| 126 | | AGCGAGCGAGCGCGC |
| SEQ ID NO: | Left ITR-13 | GCGCGCTCGCTCGCTCACTGAGGCCGCCCGAAACGGGCGTCGGGCGACCTTTGGTCGCCCGGCCTCAGTGAG |
| 127 | | CGAGCGAGCGCGC |
| SEQ ID NO: | Right ITR-13 | GCGCGCTCGCTCGCTCACTGAGGCCGGGCGACCAAAGGTCGCCCGACGCCCGTTTCGGGCGGCCTCAGTGAG |
| 128 | | CGAGCGAGCGCGC |
| SEQ ID NO: | Left ITR-14 | GCGCGCTCGCTCGCTCACTGAGGCCGCCCAAAGGGCGTCGGGCGACCTTTGGTCGCCCGGCCTCAGTGAGCG |
| 129 | | AGCGAGCGCGC |
| SEQ ID NO: | Right ITR-14 | GCGCGCTCGCTCGCTCACTGAGGCCGGGCGACCAAAGGTCGCCCGACGCCCTTTGGGCGGCCTCAGTGAGCG |
| 130 | | AGCGAGCGCGC |
| SEQ ID NO: | Left ITR-15 | GCGCGCTCGCTCGCTCACTGAGGCCGCCAAAGGCGTCGGGCGACCTTTGGTCGCCCGGCCTCAGTGAGCGAG |
| 131 | | CGAGCGCGC |
| SEQ ID NO: | Right ITR-15 | GCGCGCTCGCTCGCTCACTGAGGCCGGGCGACCAAAGGTCGCCCGACGCCTTTGGCGGCCTCAGTGAGCGAG |
| 132 | | CGAGCGCGC |
| SEQ ID NO: | Left ITR-16 | GCGCGCTCGCTCGCTCACTGAGGCCGCAAAGCGTCGGGCGACCTTTGGTCGCCCGGCCTCAGTGAGCGAGCG |
| 133 | | AGCGCGC |
| SEQ ID NO: | Right ITR-16 | GCGCGCTCGCTCGCTCACTGAGGCCGGGCGACCAAAGGTCGCCCGACGCTTTGCGGCCTCAGTGAGCGAGCG |
| 134 | | AGCGCGC |
| SEQ ID NO: | HAAT promoter | CCCTAAAATGGGCAAACATTGCAAGCAGCAAACAGCAAACACACAGCCCTCCCTGCCTGCTGACCTTGGAGC |
| 135 | | TGGGGCAGAGGTCAGAGACCTCTCTGGGCCCATGCCACCTCCAACATCCACTCGACCCCTTGGAATTTTTCGG |
| | | TGGAGAGGAGCAGAGGTTGTCCTGGCGTGGTTTAGGTAGTGTGAGAGGGGAATGACTCCTTTCGGTAAGTGC |
| | | AGTGGAAGCTGTACACTGCCCAGGCAAAGCGTCCGGGCAGCGTAGGCGGGCGACTCAGATCCCAGCCAGTGG |
| | | ACTTAGCCCCTGTTTGCTCCTCCGATAACTGGGGTGACCTTGGTTAATATTCACCAGCAGCCTCCCCCGTTGCC |
| | | CCTCTGGATCCACTGCTTAAATACGGACGAGGACAGGGCCCTGTCTCCTCAGCTTCAGGCACCACCACTGACC |
| | | TGGGACAGTGAATCCGGACTCTAAGGTAAATATAAAATTTTTAAGTGTATAATGTGTTAAACTACTGATTCTA |
| | | ATTGTTTCTCTCTTTTAGATTCCAACCTTTGGAACTG |
| SEQ ID NO: | | |
| 136-150 | | |
| SEQ ID NO: | ARSA | ATGTCCATGGGGGCACCGCGGTCCCTCCTCCTGGCCCTGGCTGCTGGCCTGGCCGTTGCCCGTCCGCCCAACA |
| 151 | (CR456383.1 | TCGTGCTGATCTTTGCCGACGACCTCGGCTATGGGGACCTGGGCTGCTATGGGCACCCCAGCTCTACCACTCC |
| | Homo sapiens | CAACCTGGACCAGCTGGCGGCGGGAGGGCTGCGGTTCACAGACTTCTACGTGCCTGTGTCTCTGTGCACACCC |
| | ARSA full | TCTAGGGCCGCCCTCCTGACCGGCCGGCTCCCGGTTCGGATGGGCATGTACCCTGGCGTCCTGGTGCCCAGCT |
| | length open | CCCGGGGGGGCCTGCCCCTGGAGGAGGTGACCGTGGCCGAAGTCCTGGCTGCCCGAGGCTACCTCACAGGAA |
| | reading frame | TGGCCGGCAAGTGGCACCTTGGGGTGGGGCCTGAGGGGGCCTTCCTGCCCCCCCATCAGGGCTTCCATCGATT |
| | (ORF) cDNA | TCTAGGCATCCCGTACTCCCACGACCAGGGCCCCTGCCAGAACCTGACCTGCTTCCCGCCGGCCACTCCTTGC |
| | clone (cDNA | GACGGTGGCTGTGACCAGGGCCTGGTCCCCATCCCACTGTTGGCCAACCTGTCCGTGGAGGCGCAGCCCCCCT |
| | clone C220RF | GGCTGCCCGGACTAGAGGCCCGCTACATGGCTTTCGCCCATGACCTCATGGCCGACGCCCAGCGCCAGGATC |
| | pGEM.ARSA.V | GCCCCTTCTTCCTGTACTATGCCTCTCACCACACCCACTACCCTCAGTTCAGTGGGCAGAGCTTTGCAGAGCGT |
| | 2)) | TCAGGCCGCGGGCCATTTGGGGACTCCCTGATGGAGCTGGATGCAGCTGTGGGGACCCTGATGACAGCCATA |
| | | GGGGACCTGGGGCTGCTTGAAGAGACGCTGGTCATCTTCACTGCAGACAATGGACCTGAGACCATGCGTATG |
| | | TCCCGAGGCGGCTGCTCCGGTCTCTTGCGGTGTGGAAAGGGAACGACCTACGAGGGCGGTGTCCGAGAGCCT |
| | | GCCTTGGCCTTCTGGCCAGGTCATATCGCTCCCGGCGTGACCCACGAGCTGGCCAGCTCCCTGGACCTGCTGC |
| | | CTACCCTGGCAGCCCTGGCTGGGGCCCCACTGCCCAATGTCACCTTGGATGGCTTTGACCTCAGCCCCCTGCT |
| | | GCTGGGCACAGGCAAGAGCCCTCGGCAGTCTCTCTTCTTCTACCCGTCCTACCCAGACGAGGTCCGTGGGGTT |
| | | TTTGCTGTGCGGACTGGAAAGTACAAGGCTCACTTCTTCACCCAGGGCTCTGCCCACAGTGATACCACTGCAG |
| | | ACCCTGCCTGCCACGCCTCCAGCTCTCTGACTGCTCATGAGCCCCCGCTGCTCTATGACCTGTCCAAGGACCCT |
| | | GGTGAGAACTACAACCTGCTGGGGGGTGTGGCCGGGGCCACCCCAGAGGTGCTGCAAGCCCTGAAACAGCTT |
| | | CAGCTGCTCAAGGCCCAGTTAGACGCAGCTGTGACCTTCGGCCCCAGCCAGGTGGCCCGGGGCGAGGACCCC |
| | | GCCCTGCAGATCTGCTGTCATCCTGGCTGCACCCCCCGCCCAGCTTGCTGCCATTGCCCAGATCCCCATGCCTG |
| | | A |
| SEQ ID NO: | I2S | ATGCCGCCACCCCGGACCGGCCGAGGCCTTCTCTGGCTGGGTCTGGTTCTGAGCTCCGTCTGCGTCGCCCTCG |
| 152 | (Genbank Homo | GATCCGAAACGCAGGCCAACTCGACCACAGATGCTCTGAACGTTCTTCTCATCATCGTGGATGACCTGCGCCC |
| | sapiens iduronate | CTCCCTGGGCTGTTATGGGGATAAGCTGGTGAGGTCCCCAAATATTGACCAACTGGCATCCCACAGCCTCCTC |
| | 2-sulfatase | TTCCAGAATGCCTTTGCGCAGCAAGCAGTGTGCGCCCCGAGCCGCGTTTCTTTCCTCACTGGCAGGAGACCTG |
| | (IDS), | ACACCACCCGCCTGTACGACTTCAACTCCTACTGGAGGGTGCACGCTGGAAACTTCTCCACCATCCCCCAGTA |
| | RefSeqGene on | CTTCAAGGAGAATGGCTATGTGACCATGTCGGTGGGAAAAGTCTTTCACCCTGGGATATCTTCTAACCATACC |
| | chromosome X) | GATGATTCTCCGTATAGCTGGTCTTTTCCACCTTATCATCCTTCCTCTGAGAAGTATGAAAACACTAAGACATG |
| | | TCGAGGGCCAGATGGAGAACTCCATGCCAACCTGCTTTGCCCTGTGGATGTGCTGGATGTTCCCGAGGGCACC |
| | | TTGCCTGACAAACAGAGCACTGAGCAAGCCATACAGTTGTTGGAAAAGATGAAAACGTCAGCCAGTCCTTTC |
| | | TTCCTGGCCGTTGGGTATCATAAGCCACACATCCCCTTCAGATACCCCAAGGAATTTCAGAAGTTGTATCCCT |
| | | TGGAGAACATCACCCTGGCCCCCGATCCCGAGGTCCCTGATGGCCTACCCCCTGTGGCCTACAACCCCTGGAT |
| | | GGACATCAGGCAACGGGAAGACGTCCAAGCCTTAAACATCAGTGTGCCGTATGGTCCAATTCCTGTGGACTTT |
| | | CAGCGGAAAATCCGCCAGAGCTACTTTGCCTCTGTGTCATATTTGGATACACAGGTCGGCCGCCTCTTGAGTG |
| | | CTTTGGACGATCTTCAGCTGGCCAACAGCACCATCATTGCATTTACCTCGGATCATGGGTGGGCTCTAGGTGA |
| | | ACATGGAGAATGGGCCAAATACAGCAATTTTGATGTTGCTACCCATGTTCCCCTGATATTCTATGTTCCTGGA |
| | | AGGACGGCTTCACTTCCGGAGGCAGGCGAGAAGCTTTTCCCTTACCTCGACCCTTTTGATTCCGCCTCACAGT |
| | | TGATGGAGCCAGGCAGGCAATCCATGGACCTTGTGGAACTTGTGTCTCTTTTTCCCACGCTGGCTGGACTTGC |
| | | AGGACTGCAGGTTCCACCTCGCTGCCCCGTTCCTTCATTTCACGTTGAGCTGTGCAGAGAAGGCAAGAACCTT |
| | | CTGAAGCATTTTCGATTCCGTGACTTGGAAGAGGATCCGTACCTCCCTGGTAATCCCCGTGAACTGATTGCCT |
| | | ATAGCCAGTATCCCCGGCCTTCAGACATCCCTCAGTGGAATTCTGACAAGCCGAGTTTAAAAGATATAAAGAT |
| | | CATGGGCTATTCCATACGCACCATAGACTATAGGTATACTGTGTGGGTTGGCTTCAATCCTGATGAATTTCTA |
| | | GCTAACTTTTCTGACATCCATGCAGGGGAACTGTATTTTGTGGATTCTGACCCATTGCAGGATCACAATATGT |
| | | ATAATGATTCCCAAGGTGGAGATCTTTTCCAGTTGTTGATGCCTTGA |
| SEQ ID NO: | RBE-1 | GCGCGCTCGCTCGCTC |
| 301 | | |
| SEQ ID NO: | Spacer | ACTGAGGC |
| 302 | | |
| SEQ ID NO: | Loop Arm | CGGGCGACCAAAGGTCGCCCGA |
| 303 | | |
| SEQ ID NO: | Truncated Arm | CGCCCGGGCG |
| 304 | | |
| SEQ ID NO: | Spacer | GCCTCAGT |
| 305 | Complement | |
| SEQ ID NO: | RBE-2 | GAGCGAGCGAGCGCGC |
| 306 | | |
| SEQ ID NO: | | |
| 307 | | |
| SEQ ID NO: | SV40 enhancer | GGTGTGGAAAGTCCCCAGGCTCCCCAGCAGGCAGAAGTATGCAAAGCATGCATCTCAATTAGTCAGCAACCAGGTGTGGAAAGTCCCC |
| 308 | | AGGCTCCCCAGCAGGCAGAAGTATGCAAAGCATGCATCTCAATTAGTCAGCAACCATAGTCCCGCCCCTAACTCCGCCCATCCCGCCCC |
| | | TAACTCCGCCCAGTTCCGCCCATTCTCCGCCCCATGGCTGACTAATTTTTTTTATTTATGCAGAGGCCGAGGCCGCCTCGGCCTCTGAGCT |
| | | ATTCCAGAAGTAGTGAGGAGGCTTTTTTGGAGGCCTAGGCTTTTGCAAA |
| SEQ ID NO: | CMV enhancer | TAGTAATCAATTACGGGGTCATTAGTTCATAGCCCATATATGGAGTTCCGCGTTACATAACTTACGGTAAATGGCCCGCCTGGCTGACC |
| 309 | | GCCCAACGACCCCCGCCCATTGACGTCAATAATGACGTATGTTCCCATAGTAACGCCAATAGGGACTTTCCATTGACGTCAATGGGTGG |
| | | AGTATTTACGGTAAACTGCCCACTTGGCAGTACATCAAGTGTATCATATGCCAAGTACGCCCCCTATTGACGTCAATGACGGTAAATGG |
| | | CCCGCCTGGCATTATGCCCAGTACATGACCTTATGGGACTTTCCTACTTGGCAGTACATCTACGTATTAGTCATCGCTATTACCATGGTG |
| | | ATGCGGTTTTGGCAGTACATCAATGGGCGTGGATAGCGGTTTGACTCACGGGGATTTCCAAGTCTCCACCCCATTGACGTCAATGGGA |
| | | GTTTGTTTTGGCACCAAAATCAACGGGACTTTCCAAAATGTCGTAACAACTCCGCCCCATTGACGCAAATGGGCGGTAGGCGTGTACGG |
| | | TGGGAGGTCTATATAAGCAGAGCTGGTTTAGTGAACCGTCAG |
| SEQ ID NO: | Rat EF1-α | GGAGCCGAGAGTAATTCATACAAAAGGAGGGATCGCCTTCGCAAGGGGAGAGCCCAGGGACCGTCCCTAAA |
| 310 | promoter | TTCTCACAGACCCAAATCCCTGTAGCCGCCCCACGACAGCGCGAGGAGCATGCGCCCAGGGCTGAGCGCGGG |
| | (Rattus | TAGATCAGAGCACACAAGCTCACAGTCCCCGGCGGTGGGGGGAGGGGCGCGCTGAGCGGGGGCCAGGGAGC |
| | norvegicus BAC | TGGCGCGGGGCAAACTGGGAAAGTGGTGTCGTGTGCTGGCTCCGCCCTCTTCCCGAGGGTGGGGGAGAACGG |
| | CH230-35L12 | TATATAAGTGCGGTAGTCGCCTTGGACGTTCTTTTTCGCAACGGGTTTGCCGTCAGAACGCAGGTGAGTGGCG |
| | (Children′s | GGTGTGGCTTCCGCGGGCCCCGGAGCTGGAGCCCTGCTCTGAGCGGGCCGGGCTGATATGCGAGTGTCGTCC |
| | Hospital | GCAGGGTTTAGCTGTGAGCATTCCCACTTCGAGTGGCGGGCGGTGCGGGGGTGAGAGTGCGAGGCCTAGCGG |
| | Oakland | CAACCCCGTAGCCTCGCCTCGTGTCCGGCTTGAGGCCTAGCGTGGTGTCCGCCGCCGCGTGCCACTCCGGCCG |
| | Research | CACTATGCGTTTTTTGTCCTTGCTGCCCTCGATTGCCTTCCAGCAGCATGGGCTAACAAAGGGAGGGTGTGGG |
| | Institute) | GCTCACTCTTAAGGAGCCCATGAAGCTTACGTTGGATAGGAATGGAAGGGCAGGAGGGGCGACTGGGGCCCG |
| | complete | CCCGCCTTCGGAGCACATGTCCGACGCCACCTGGATGGGGCGAGGCCTGTGGCTTTCCGAAGCAATCGGGCG |
| | sequenceSequence | TGAGTTTAGCCTACCTGGGCCATGTGGCCCTAGCACTGGGCACGGTCTGGCCTGGCGGTGCCGCGTTCCCTTG |
| | ID: | CCTCCCAACAAGGGTGAGGCCGTCCCGCCCGGCACCAGTTGCTTGCGCGGAAAGATGGCCGCTCCCGGGGCC |
| | gi\|49615137\| | CTGTTGCAAGGAGCTCAAAATGGAGGACGCGGCAGCCCGGTGGAGCGGGCGGGTGAGTCACCCACACAAAG |
| | AC097023.6) | GAAGAGGGCCTTGCCCCTCGCCGGCCGCTGCTTCCTGTGACCCCGTGGTCTATCGGCCGCATAGTCACCTCGG |
| | | GCTTCTCTTGAGCACCGCTCGTCGCGGCGGGGGGAGGGGATCTAATGGCGTTGGAGTTTGTTCACATTTGGTG |
| | | GGTGGAGACTAGTCAGGCCAGCCTGGCGCTGGAAGTCATTCTTGGAATTTGCCCCTTTGAGTTTGGAGCGAGG |
| | | CTAATTCTCAAGCCTCTTAGCGGTTCAAAGGTATTTTCTAAACCCGTTTCCAGGTGTTGTGAAAGCCACCGCTA |
| | | ATTCAAAGCAA |
| SEQ ID NO: | VH1-02 | MDWTWRILFLVAAATGAHS |
| 313 | secretory leader | |
| SEQ ID NO: | VK A26 | MLPSQLIGFLLLWVPASRG |
| 314 | secretory leader | |
| SEQ ID NO: | SV40 virus large | PKKKRKV |
| 315 | T-antigen | |
| SEQ ID NO: | nucleoplasmin | KRPAATKKAGQAKKKK |
| 316 | | |
| SEQ ID NO: | c-myc | PAAKRVKLD |
| 317 | | |
| SEQ ID NO: | c-myc | RQRRNELKRSP |
| 318 | | |
| SEQ ID NO: | hRNPA1 M9 | NQSSNFGPMKGGNFGGRSSGPYGGGGQYFAKPRNQGGY |
| 319 | | |
| SEQ ID NO: | IBB domain | RMRIZFKNKGKDTAELRRRRVEVSVELRKAKKDEQILKRRNV |
| 320 | from importin- | |
| | alpha | |
| SEQ ID NO: | myoma T protein | VSRKRPRP |
| 321 | | |
| SEQ ID NO: | human p53 | PQPKKKPL |
| 323 | | |
| SEQ ID NO: | mouse c-abl IV | SALIKKKKKMAP |
| 324 | | |
| SEQ ID NO: | influenza virus | DRLRR |
| 325 | NS1 | |
| SEQ ID NO: | influenza virus | PKQKKRK |
| 326 | NS1 | |
| SEQ ID NO: | Hepatitis virus | RKLKKKIKKL |
| 327 | delta antigen | |
| SEQ ID NO: | mouse Mx1 | REKKKFLKRR |
| 328 | protein | |
| SEQ ID NO: | human | KRKGDEVDGVDEVAKKKSKK |
| 329 | poly(ADP-ribose) | |
| | polymerase | |
| SEQ ID NO: | steroid hormone | RKCLQAGMNLEARKTKK |
| 330 | receptors | |
| | (human) | |
| | glucocorticoid | |
| SEQ ID NO: | | |
| 331-499 | | |
| SEQ ID: 500 | | GCCCGCTGGTTTCCAGCGGGCTGCGGGCCCGAAACGGGCCCGC |
| SEQ ID: 501 | | CGGGCCCGTGCGGGCCCAAAGGGCCCGC |
| SEQ ID: 502 | | GCCCGGGCACGCCCGGGTTTCCCGGGCG |
| SEQ ID: 503 | | CGTGCGGGCCCAAAGGGCCCGC |

RIGHT side ITR Selected Polynucleotide Sequences:

| | C-C′ | | B-B′ | Whole sequence |
|---|---|---|---|---|
| WT-ITR- | CGGGCGACCAAAGGTCGCCC | A | CGCCCGGGCTTTGCCC | CGGGCGACCAAAGGTCGCCCG |
| R | G (SEQ ID: 504) | | GGGC (SEQ ID: 505) | ACGCCCGGGCTTTGCCCGGGC |
| | | | (SEQ ID: 506) | |
| TTX1-R | CGGGCGACCAAAGGTCGCCC | A | CGCCCGGGC(TTTGCC | CGGGCGACCAAAGGTCGCCCG |
| (ITR- | G (SEQ ID: 507) | | CG)GGC (SEQ ID: 508) | ACGCCCGGGCGGC |
| folding | | | | (SEQ ID: 509) |
| prediction) | | | | |
| Kotin 2 | CGGG(CGACCAAAGGTC)GC | A | CGCCCGGGCTTTGCCC | CGGGGCCCGACGCCCGGGCTTT |
| (p.11) | CCG (SEQ ID: 510) | | GGGC (SEQ ID: 511) | GCCCGGGC (SEQ ID: 512) |
| Kotin 2 | CGGG(CGACCAAAGGTCG)C | A | CGCCCGGGCTTTGCCC | CGGGCCCGACGCCCGGGCTTTG |
| (p.11) | CCG (SEQ ID: 513) | | GGGC (SEQ ID: 514) | CCCGGGC (SEQ ID: 515) |
| Kotin 2 | [CGGGCGACCAAAGGTCGCC | A | CGCCCGGGCTTTGCCC | |
| (p.11) | CG] all or partial deletions within | | | |
| | GGGC (SEQ ID: 517) | | | |
| | the square brackets can be used to | | | |
| | create asymmetric interrupted self- | | | |
| | complementary sequences; (SEQ | | | |
| | ID: 516) | | | |

LEFT side ITR Selected Polynucleotide Sequences:

| | C-C′ | | B-B′ | Whole sequence |
|---|---|---|---|---|
| WT-ITR- | GCCCGGGCAAAGCCCGGGCG | T | CGGGCGACCTTTGGTC | GCCCGGGCAAAGCCCGGGCGT |
| L | (SEQ ID: 518) | | GCCCG (SEQ ID: 519) | CGGGCGACCTTTGGTCGCCCG |
| | | | | (SEQ ID: 520) |
| SEQ ID: | [GCCCGGGCAAA]GCCCGGGC | T | CGGGCGACCTTTGGTC | GCCCGGGCGTCGGGCGACCTTT |
| 22 | G (SEQ ID: 521) | | GCCCG (SEQ ID: 522) | GGTCGCCCG (SEQ ID: 522) |
| TTX1-L | GCC(CGGGCAAA)GCCCGGGC | T | CGGGCGACCTTTGGTC | GCCGCCCGGGCGACGGGCGAC |
| (ITR- | G (SEQ ID: 523) | | GCCCG (SEQ ID: 524) | CTTTGGTCGCCCG (SEQ ID: 525) |
| folding | | | | |
| prediction) | | | | |
| Kotin 2 | GCCC(GGGCAAAGCCC)GGGC | T | CGGGCGACCTTTGGTC | GCCCGGGCGTCGGGCGACCTTT |
| (p.11) | G (SEQ ID: 526) | | GCCCG (SEQ ID: 527) | GGTCGCCCG (SEQ ID: 528) |
| Kotin 2 | [GCCCGGGCAAAGCCCGGGC | T | CGGGCGACCTTTGGTC | |
| (p.11) | G] all or partial deletions within | | | |
| | GCCCG (SEQ ID: 529) | | | |
| | the square brackets can be used to | | | |
| | create asymmetric interrupted self- | | | |
| | complementary sequences; (SEQ | | | |
| | ID: 528) | | | |

| | | |
|---|---|---|
| SEQ ID NO: | NM_004895.1 | CAGGGCAGCCTTCAGTCTGATTCAGGAGAACGAGGTCCTCTTCACCATGTGCTTCATCCCCCTGGTCTGCTGGATCGTGTGCACT |
| 530 | | GGACTGAAACAGCAGATGGAGAGTGGCAAGAGCCTTGCCCAGACATCCAAGACCTCCACCGCGGTGTACGTCTTCTTCCTTTCCA |
| | | GTTTGCTGCAGCCCCGGGGAGGGAGCCAGGAGCACGGCCTCTGCGCCCACCTCTGGGGGCTCTGCTCTTTGGCTGCAGATGGAAT |
| | | CTGGAACCAGAAAATCCTGTTTGAAGAGTCCGACCTCAGGAATCATGGACTGCAGAAGGCGGATGTGTCTGCTTTCCTGAGGATG |
| | | AACCTGTTCCAAAAGGAAGTGGACTGCGAGAAGTTCTACAGCTTCATCCACATGACTTTCCAGGAGTTCTTTGCCGCCATGTACT |
| | | ACCTGCTGGAAGAGGAAAAGGAAGGAAGGACGAACGTTCCAGGGAGTCGTTTGAAGCTTCCCAGCCGAGACGTGACAGTCCTTCT |
| | | GGAAAACTATGGCAAATTCGAAAAGGGGTATTTGATTTTTGTTGTACGTTTCCTCTTTGGCCTGGTAAACCAGGAGAGGACCTCC |
| | | TACTTGGAGAAGAAATTAAGTTGCATGATCTCTCAGCAAATCAGGCTGGAGCTGCTGAAATGGATTGAAGTGAAAGCCAAAGCTA |
| | | AAAAGCTGCATGATCAGCCCAGCCAGCTGGAATTGTTCTACTGTTTGTACGAGATGCAGGAGGAGGACTTCGTGCAAAGGGCCAT |
| | | GGACTATTTCCCCAAGATTGAGATCAATCTCTCCACCAGAATGGACCACATGGTTTCCTCCTTTTGCATTGAGAACTGTCATCGG |
| | | GTGGAGTCACTGTCCCTGGGGTTTCTCCATAACATGCCCAAGGAGGAAGAGGAGGAGGAAAAGGAAGGCCGACACCTTGATATGG |
| | | TGCAGTGTGTCCTCCCAAGCTCCTCTCATGCTGCCTGTTCTCATGGGTTGGGGCGCTGTGGCCTCTCCCATGAGTGCTGCTTCGA |
| | | CATCTCCTTGGTCCTCAGCAGCAACCAGAAGCTGGTGGAGCTGGACCTGAGTGACAACGCCCTCGGTGACTTCGGAATCAGACTT |
| | | CTGTGTGTGGGACTGAAGCACCTGTTGTGCAATCTGAAGAAGCTCTGGTTGGTGAATTCTGCCTTACGTCAGTCTGTTGTTCAGC |
| | | TTTGTCCTCGGTACTCAGCACTAATCAGAATCTCACGCACCTTTACTGCGAGGCAACACTCTCGGAGACAAGGGATCAAACTACT |
| | | CTGTGAGGGACTCTTGCACCCCGACTGCAAGCTTCAGGTGTTGGAATTAGACAACTGCAACCTCACGTCACACTGCTGCTGGGAT |
| | | CTTTCCACACTTCTGACCTCCAGCCAGAGCCTGCGAAAGCTGAGCCTGGGCAACAATGACCTGGGCGACCTGGGGGTCATGATGT |
| | | TCTGTGAAGTGCTGAAACAGCAGAGCTGCCTCCTGCAGAACCTGGGGTTGTCTGAAATGTATTTCAATTATGAGACAAAAAGTGC |
| | | GTTAGAAACACTTCAAGAAGAAAAGCCTGAGCTGACCGTCGTCTTTGAGCCTTCTTGGTAGGAGTGGAAACGGGGCTGCCAGACG |
| | | CCAGTGTTCTCCGGTCCCTCCAGCTGGGGGCCCTCAGGTGGAGAGAGCTGCGATCCATCCAGGCCAAGACCACAGCTCTGTGATC |
| | | CTTCCGGTGGAGTGTCGGAGAAGAGAGCTTGCCGACGATGCCTTCCTGTGCAGAGCTTGGGCATCTCCTTTACGCCAGGGTGAGG |
| | | AAGACACCAGGACAATGACAGCATCGGGTGTTGTTGTCATCACAGCGCCTCAGTTAGAGGATGTTCCTCTGGTGACCTCATGTAA |
| | | TTAGCTCATTCAATAAAGCACTTTCTTTATTTTTCTCTTCTCTGTCTAACCTTCTTTTTCCTATCTTTTTTTCTTCTTTGTTCTG |
| | | TTTACTTTTGCTCATATCATCATTCCCGCTATCTTTCTATTAACTGACCATAACACAGAACTAGTTGACTATATATTATGTTGAA |
| | | ATTTTATGGCAGCTATTTATTTATTTAAATTTTTTGTAATAGTTTTGTTTTCTAATAAGAAAAATCCATGCTTTTTGTAGCTGGT |
| | | TGAAAATTCAGGAATATGTAAAACTTTTTGGTATTTAATTAAATTGATTCCTTTTCTTAATTTT |
| | | AAAAAAAA |
| SEQ ID NO: | NM_183395 | GTTCCTGAGGCTGGCATCTGGATGAGGAAACTGAAGTTGAGGAATAGTGAAGAGTTTGTCCAATGTCATAGCCCCGTAATCAACG |
| 531 | | GGACAAAAATTTTCTTGCTGATGGGTCAAGATGGCATCGTGAAGTGGTTGTTCACCGTAAACTGTAATACAATCCTGTTTATGGA |
| | | TTTGTTTGCATATTTTTCCCTCCATAGGGAAACCTTTCTTCCATGGCTCAGGACACACTCCTGGATCGAGCCAACAGGAGAACTT |
| | | TCTGGTAAGCATTTGGCTAACTTTTTTTTTTTTGAGATGGAGTCTTGCTGTGTCGCCTAGGCTGGAGTGCAGTGGCGTGATCTTG |
| | | GCTCACTGCAGCCTCCACTTCCCGGGTTCAATCAATTCTCCTACCTCAACTTCCTGAGTAGCTGGGATTACAGGCGCCCGCCACC |
| | | ACACCCGGCTCATTTTTGTACTTTTAGTAGAGACACAGTTTTGCCATGTTGGCCAGGCTGGTCTTGAATTCCTCAGCTCAGGTGA |
| | | TCTGCCTGCCTTGGCCTCTCAAAGTGCTGGGATTACAGGCGTGAGCCACTGTGCCCGGCCTTGGCTAACTTTTCAAAATTAAAGA |
| | | TTTTGACTTGTTACAGTCATGTGACATTTTTTTCTTTCTGTTTGCTGAGTTTTTGATAATTTATATCTCTCAAAGTGGAGACTTT |
| | | AAAAAAGACTCATCCGTGTGCCGTGTTCACTGCCTGGTATCTTAGTGTGGACCGAAGCCTAAGGACCCTGAAAACAGCTGCAGAT |
| | | GAAGATGGCAAGCACCCGCTGCAAGCTGGCCAGGTACCTGGAGGACCTGGAGGATGTGGACTTGAAGAAATTTAAGATGCACTTA |
| | | GAGGACTATCCTCCCCAGAAGGGCTGCATCCCCCTCCCGAGGGGTCAGACAGAGAAGGCAGACCATGTGGATCTAGCCACGCTAA |
| | | TGATCGACTTCAATGGGGAGGAGAAGGCGTGGGCCATGGCCGTGTGGATCTTCGCTGCGATCAACAGGAGAGACCTTTATGAGAA |
| | | AGCAAAAAGAGATGAGCCGAAGTGGGGTTCAGATAATGCACGTGTTTCGAATCCCACTGTGATATGCCAGGAAGACAGCATTGAA |
| | | GAGGAGTGGATGGGTTTACTGGAGTACCTTTCGAGAATCTCTATTTGTAAAATGAAGAAAGATTACCGTAAGAAGTACAGAAAGT |
| | | ACGTGAGAAGCAGATTCCAGTGCATTGAAGACAGGAATGCCCGTCTGGGTGAGAGTGTGAGCCTCAACAAACGCTACACACGACT |
| | | GCGTCTCATCAAGGAGCACCGGAGCCAGCAGGAGAGGGAGCAGGAGCTTCTGGCCATCGGCAAGACCAAGACGTGTGAGAGCCCC |
| | | GTGAGTCCCATTAAGATGGAGTTGCTGTTTGACCCCGATGATGAGCATTCTGAGCCTGTGCACACCGTGGTGTTCCAGGGGGCGG |
| | | CAGGGATTGGGAAAACAATCCTGGCCAGGAAGATGATGTTGGACTGGGCGTCGGGGACACTCTACCAAGACAGGTTTGACTATCT |
| | | GTTCTATATCCACTGTCGAGAGGTGAGCCTTGTGACACAGAGGAGCCTGGGGGACCTGATCATGAGCTGCTGCCCCGACCCAAAC |
| | | CCACCCATCCACAAGATCGTGAGAAAACCCTCCAGAATCCTCTTCCTCATGGACGGCTTCGATGAGCTGCAAGGTGCCTTTGACG |
| | | AGCACATAGGACCGCTCTGCACTGACTGGCAGAAGGCCGAGCGGGGAGACATTCTCCTGAGCAGCCTCATCAGAAAGAAGCTGCT |
| | | TCCCGAGGCCTCTCTGCTCATCACCACGAGACCTGTGGCCCTGGAGAAACTGCAGCACTTGCTGGACCATCCTCGGCATGTGGAG |
| | | ATCCTGGGTTTCTCCGAGGCCAAAAGGAAAGAGTACTTCTTCAAGTACTTCTCTGATGAGGCCCAAGCCAGGGCAGCCTTCAGTC |
| | | TGATTCAGGAGAACGAGGTCCTCTTCACCATGTGCTTCATCCCCCTGGTCTGCTGGATCGTGTGCACTGGACTGAAACAGCAGAT |
| | | GGAGAGTGGCAAGAGCCTTGCCCAGACATCCAAGACCACCACCGCGGTGTACGTCTTCTTCCTTTCCAGTTTGCTGCAGCCCCGG |
| | | GGAGGGAGCCAGGAGCACGGCCTCTGCGCCCACCTCTGGGGGCTCTGCTCTTTGGCTGCAGATGGAATCTGGAACCAGAAAATCC |
| | | TGTTTGAGGAGTCCGACCTCAGGAATCATGGACTGCAGAAGGCGGATGTGTCTGCTTTCCTGAGGATGAACCTGTTCCAAAAGGA |
| | | AGTGGACTGCGAGAAGTTCTACAGCTTCATCCACATGACTTTCCAGGAGTTCTTTGCCGCCATGTACTACCTGCTGGAAGAGGAA |
| | | AAGGAAGGAAGGACGAACGTTCCAGGGAGTCGTTTGAAGCTTCCCAGCCGAGACGTGACAGTCCTTCTGGAAAACTATGGCAAAT |
| | | TCGAAAAGGGGTATTTGATTTTTGTTGTACGTTTCCTCTTTGGCCTGGTAAACCAGGAGAGGACCTCCTACTTGGAGAAGAAATT |
| | | AAGTTGCAAGATCTCTCAGCAAATCAGGCTGGAGCTGCTGAAATGGATTGAAGTGAAAGCCAAAGCTAAAAAGCTGCAGATCCAG |
| | | CCCAGCCAGCTGGAATTGTTCTACTGTTTGTACGAGATGCAGGAGGAGGACTTCGTGCAAAGGGCCATGGACTATTTCCCCAAGA |
| | | TTGAGATCAATCTCTCCACCAGAATGGACCACATGGTTTCTTCCTTTTGCATTGAGAACTGTCATCGGGTGGAGTCACTGTCCCT |
| | | GGGGTTTCTCCATAACATGCCCAAGGAGGAAGAGGAGGAGGAAAAGGAAGGCCGACACCTTGATATGGTGCAGTGTGTCCTCCCA |
| | | AGCTCCTCTCATGCTGCCTGTTCTCATGGGTTGGGGCGCTGTGGCCTCTCGCATGAGTGCTGCTTCGACATCTCCTTGGTCCTCA |
| | | GCAGCAACCAGAAGCTGGTGGAGCTGGACCTGAGTGACAACGCCCTCGGTGACTTCGGAATCAGACTTCTGTGTGTGGGACTGAA |
| | | GCACCTGTTGTGCAATCTGAAGAAGCTCTGGTTGGTGAATTCTGGCCTTACGTCAGTCTGTTGTTCAGCTTTGTCCTCGGTACTC |
| | | AGCACTAATCAGAATCTCACGCACCTTTACCTGCGAGGCAACACTCTCGGAGACAAGGGGATCAAACTACTCTGTGAGGGACTCT |
| | | TGCACCCCGACTGCAAGCTTCAGGTGTTGGAATTAGACAACTGCAACCTCACGTCACACTGCTGCTGGGATCTTTCCACACTTCT |
| | | GACCTCCAGCCAGAGCCTGCGAAAGCTGAGCCTGGGCAACAATGACCTGGGCGACCTGGGGGTCATGATGTTCTGTGAAGTGCTG |
| | | AAACAGCAGAGCTGCCTCCTGCAGAACCTGGGGTTGTCTGAAATGTATTTCAATTATGAGACAAAAAGTGCGTTAGAAACACTTC |
| | | AAGAAGAAAAGCCTGAGCTGACCGTCGTCTTTGAGCCTTCTTGGTAGGAGTGGAAACGGGGCTGCCAGACGCCAGTGTTCTCCGG |
| | | TCCCTCCAGCTGGGGGCCCTCAGGTGGAGAGAGCTGCGATCCATCCAGGCCAAGACCACAGCTCTGTGATCCTTCCGGTGGAGTG |
| | | TCGGAGAAGAGAGCTTGCCGACGATGCCTTCCTGTGCAGAGCTTGGGCATCTCCTTTACGCCAGGGTGAGGAAGACACCAGGACA |
| | | ATGACAGCATCGGGTGTTGTTGTCATCACAGCGCCTCAGTTAGAGGATGTTCCTCTTGGTGACCTCATGTAATTAGCTCATTCAA |
| | | TAAAGCACTTTCTTTATTTT |
| SEQ ID NO: | NM_001079821 | GTTCCTGAGGCTGGCATCTGGGGAAACCTTTCTTCCATGGCTCAGGACACACTCCTGGATCGAGCCAACAGGAGAACTTTCTGTG |
| 532 | | TGGACCGAAGCCTAAGGACCCTGAAAACAGCTGCAGATGAAGATGGCAAGCACCCGCTGCAAGCTGGCCAGGTACCTGGAGGACC |
| | | TGGAGGATGTGGACTTGAAGAAATTTAAGATGCACTTAGAGGACTATCCTCCCCAGAAGGGCTGCATCCCCCTCCCGAGGGGTCA |
| | | GACAGAGAAGGCAGACCATGTGGATCTAGCCACGCTAATGATCGACTTCAATGGGGAGGAGAAGGCGTGGGCCATGGCCGTGTGG |
| | | ATCTTCGCTGCGATCAACAGGAGAGACCTTTATGAGAAAGCAAAAAGAGATGAGCCGAAGTGGGGTTCAGATAATGCACGTGTTT |
| | | CGAATCCCACTGTGATATGCCAGGAAGACAGCATTGAAGAGGAGTGGATGGGTTTACTGGAGTACCTTTCGAGAATCTCTATTTG |
| | | TAAAATGAAGAAAGATTACCGTAAGAAGTACAGAAAGTACGTGAGAAGCAGATTCCAGTGCATTGAAGACAGGAATGCCCGTCTG |
| | | GGTGAGAGTGTGAGCCTCAACAAACGCTACACACGACTGCGTCTCATCAAGGAGCACCGGAGCCAGCAGGAGAGGGAGCAGGAGC |
| | | TTCTGGCCATCGGCAAGACCAAGACGTGTGAGAGCCCCGTGAGTCCCATTAAGATGGAGTTGCTGTTTGACCCCGATGATGAGCA |
| | | TTCTGAGCCTGTGCACACCGTGGTGTTCCAGGGGGCGGCAGGGATTGGGAAAACAATCCTGGCCAGGAAGATGATGTTGGACTGG |
| | | GCGTCGGGGACACTCTACCAAGACAGGTTTGACTATCTGTTCTATATCCACTGTCGAGAGGTGAGCCTTGTGACACAGAGGAGCC |
| | | TGGGGGACCTGATCATGAGCTGCTGCCCCGACCCAAACCCACCCATCCACAAGATCGTGAGAAAACCCTCCAGAATCCTCTTCCT |
| | | CATGGACGGCTTCGATGAGCTGCAAGGTGCCTTTGACGAGCACATAGGACCGCTCTGCACTGACTGGCAGAAGGCCGAGCGGGGA |
| | | GACATTCTCCTGAGCAGCCTCATCAGAAAGAAGCTGCTTCCCGAGGCCTCTCTGCTCATCACCACGAGACCTGTGGCCCTGGAGA |
| | | AACTGCAGCACTTGCTGGACCATCCTCGGCATGTGGAGATCCTGGGTTTCTCCGAGGCCAAAAGGAAAGAGTACTTCTTCAAGTA |
| | | CTTCTCTGATGAGGCCCAAGCCAGGGCAGCCTTCAGTCTGATTCAGGAGAACGAGGTCCTCTTCACCATGTGCTTCATCCCCCTG |
| | | GTCTGCTGGATCGTGTGCACTGGACTGAAACAGCAGATGGAGAGTGGCAAGAGCCTTGCCCAGACATCCAAGACCACCACCGCGG |
| | | TGTACGTCTTCTTCCTTTCCAGTTTGCTGCAGCCCCGGGGAGGGAGCCAGGAGCACGGCCTCTGCGCCCACCTCTGGGGGCTCTG |
| | | CTCTTTGGCTGCAGATGGAATCTGGAACCAGAAAATCCTGTTTGAGGAGTCCGACCTCAGGAATCATGGACTGCAGAAGGCGGAT |
| | | GTGTCTGCTTTCCTGAGGATGAACCTGTTCCAAAAGGAAGTGGACTGCGAGAAGTTCTACAGCTTCATCCACATGACTTTCCAGG |
| | | AGTTCTTTGCCGCCATGTACTACCTGCTGGAAGAGGAAAAGGAAGGAAGGACGAACGTTCCAGGGAGTCGTTTGAAGCTTCCCAG |
| | | CCGAGACGTGACAGTCCTTCTGGAAAACTATGGCAAATTCGAAAAGGGGTATTTGATTTTTGTTGTACGTTTCCTCTTTGGCCTG |
| | | GTAAACCAGGAGAGGACCTCCTACTTGGAGAAGAAATTAAGTTGCAAGATCTCTCAGCAAATCAGGCTGGAGCTGCTGAAATGGA |
| | | TTGAAGTGAAAGCCAAAGCTAAAAAGCTGCAGATCCAGCCCAGCCAGCTGGAATTGTTCTACTGTTTGTACGAGATGCAGGAGGA |
| | | GGACTTCGTGCAAAGGGCCATGGACTATTTCCCCAAGATTGAGATCAATCTCTCCACCAGAATGGACCACATGGTTTCTTCCTTT |
| | | TGCATTGAGAACTGTCATCGGGTGGAGTCACTGTCCCTGGGGTTTCTCCATAACATGCCCAAGGAGGAAGAGGAGGAGGAAAAGG |
| | | AAGGCCGACACCTTGATATGGTGCAGTGTGTCCTCCCAAGCTCCTCTCATGCTGCCTGTTCTCATGGATTGGTGAACAGCCACCT |
| | | CACTTCCAGTTTTTGCCGGGGCCTCTTTTCAGTTCTGAGCACCAGCCAGAGTCTAACTGAATTGGACCTCAGTGACAATTCTCTG |
| | | GGGGACCCAGGGATGAGAGTGTTGTGTGAAACGCTCCAGCATCCTGGCTGTAACATTCGGAGATTGTGGTTGGGGCGCTGTGGCC |
| | | TCTCGCATGAGTGCTGCTTCGACATCTCCTTGGTCCTCAGCAGCAACCAGAAGCTGGTGGAGCTGGACCTGAGTGACAACGCCCT |
| | | CGGTGACTTCGGAATCAGACTTCTGTGTGTGGGACTGAAGCACCTGTTGTGCAATCTGAAGAAGCTCTGGTTGGTCAGCTGCTGC |
| | | CTCACATCAGCATGTTGTCAGGATCTTGCATCAGTATTGAGCACCAGCCATTCCCTGACCAGACTCTATGTGGGGGAGAATGCCT |
| | | TGGGAGACTCAGGAGTCGCAATTTTATGTGAAAAAGCCAAGAATCCACAGTGTAACCTGCAGAAACTGGGGTTGGTGAATTCTGG |
| | | CCTTACGTCAGTCTGTTGTTCAGCTTTGTCCTCGGTACTCAGCACTAATCAGAATCTCACGCACCTTTACCTGCGAGGCAACACT |
| | | CTCGGAGACAAGGGGATCAAACTACTCTGTGAGGGACTCTTGCACCCCGACTGCAAGCTTCAGGTGTTGGAATTAGACAACTGCA |
| | | ACCTCACGTCACACTGCTGCTGGGATCTTTCCACACTTCTGACCTCCAGCCAGAGCCTGCGAAAGCTGAGCCTGGGCAACAATGA |
| | | CCTGGGCGACCTGGGGGTCATGATGTTCTGTGAAGTGCTGAAACAGCAGAGCTGCCTCCTGCAGAACCTGGGGTTGTCTGAAATG |
| | | TATTTCAATTATGAGACAAAAAGTGCGTTAGAAACACTTCAAGAAGAAAAGCCTGAGCTGACCGTCGTCTTTGAGCCTTCTTGGT |
| | | AGGAGTGGAAACGGGGCTGCCAGACGCCAGTGTTCTCCGGTCCCTCCAGCTGGGGGCCCTCAGGTGGAGAGAGCTGCGATCCATC |
| | | CAGGCCAAGACCACAGCTCTGTGATCCTTCCGGTGGAGTGTCGGAGAAGAGAGCTTGCCGACGATGCCTTCCTGTGCAGAGCTTG |
| | | GGCATCTCCTTTACGCCAGGGTGAGGAAGACACCAGGACAATGACAGCATCGGGTGTTGTTGTCATCACAGCGCCTCAGTTAGAG |
| | | GATGTTCCTCTTGGTGACCTCATGTAATTAGCTCATTCAATAAAGCACTTTCTTTATTTT |
| SEQ ID NO: | NM_001127461 | GTTCCTGAGGCTGGCATCTGGATGAGGAAACTGAAGTTGAGGAATAGTGAAGAGTTTGTCCAATGTCATAGCCCCGTAATCAACG |
| 533 | | GGACAAAATTTTCTTGCTGATGGGTCAAGATGGCATCGTGAAGTGGTTGTTCACCGTAAACTGTAATACAATCCTGTTTATGGAT |
| | | TTGTTTGCATATTTTTCCCTCCATAGGGAAACCTTTCTTCCATGGCTCAGGACACACTCCTGGATCGAGCCAACAGGAGAACTTT |
| | | CTGGTAAGCATTTGGCTAACTTTTTTTTTTTTGAGATGGAGTCTTGCTGTGTCGCCTAGGCTGGAGTGCAGTGGCGTGATCTTGG |
| | | CTCACTGCAGCCTCCACTTCCCGGGTTCAATCAATTCTCCTACCTCAACTTCCTGAGTAGCTGGGATTACAGGCGCCCGCCACCA |
| | | CACCCGGCTCATTTTTGTACTTTTAGTAGAGACACAGTTTTGCCATGTTGGCCAGGCTGGTCTTGAATTCCTCAGCTCAGGTGAT |
| | | CTGCCTGCCTTGGCCTCTCAAAGTGCTGGGATTACAGGCGTGAGCCACTGTGCCCGGCCTTGGCTAACTTTTCAAAATTAAAGAT |
| | | TTTGACTTGTTACAGTCATGTGACATTTTTTTCTTTCTGTTTGCTGAGTTTTTGATAATTTATATCTCTCAAAGTGGAGACTTTA |
| | | AAAAAGACTCATCCGTGTGCCGTGTTCACTGCCTGGTATCTTAGTGTGGACCGAAGCCTAAGGACCCTGAAAACAGCTGCAGATG |
| | | AAGATGGCAAGCACCCGCTGCAAGCTGGCCAGGTACCTGGAGGACCTGGAGGATGTGGACTTGAAGAAATTTAAGATGCACTTAG |
| | | AGGACTATCCTCCCCAGAAGGGCTGCATCCCCCTCCCGAGGGGTCAGACAGAGAAGGCAGACCATGTGGATCTAGCCACGCTAAT |
| | | GATCGACTTCAATGGGGAGGAGAAGGCGTGGGCCATGGCCGTGTGGATCTTCGCTGCGATCAACAGGAGAGACCTTTATGAGAAA |
| | | GCAAAAAGAGATGAGCCGAAGTGGGGTTCAGATAATGCACGTGTTTCGAATCCCACTGTGATATGCCAGGAAGACAGCATTGAAG |
| | | AGGAGTGGATGGGTTTACTGGAGTACCTTTCGAGAATCTCTATTTGTAAAATGAAGAAAGATTACCGTAAGAAGTACAGAAAGTA |
| | | CGTGAGAAGCAGATTCCAGTGCATTGAAGACAGGAATGCCCGTCTGGGTGAGAGTGTGAGCCTCAACAAACGCTACACACGACTG |
| | | CGTCTCATCAAGGAGCACCGGAGCCAGCAGGAGAGGGAGCAGGAGCTTCTGGCCATCGGCAAGACCAAGACGTGTGAGAGCCCCG |
| | | TGAGTCCCATTAAGATGGAGTTGCTGTTTGACCCCGATGATGAGCATTCTGAGCCTGTGCACACCGTGGTGTTCCAGGGGGCGGC |
| | | AGGGATTGGGAAAACAATCCTGGCCAGGAAGATGATGTTGGACTGGGCGTCGGGGACACTCTACCAAGACAGGTTTGACTATCTG |
| | | TTCTATATCCACTGTCGAGAGGTGAGCCTTGTGACACAGAGGAGCCTGGGGGACCTGATCATGAGCTGCTGCCCCGACCCAAACC |
| | | CACCCATCCACAAGATCGTGAGAAAACCCTCCAGAATCCTCTTCCTCATGGACGGCTTCGATGAGCTGCAAGGTGCCTTTGACGA |
| | | GCACATAGGACCGCTCTGCACTGACTGGCAGAAGGCCGAGCGGGGAGACATTCTCCTGAGCAGCCTCATCAGAAAGAAGCTGCTT |
| | | CCCGAGGCCTCTCTGCTCATCACCACGAGACCTGTGGCCCTGGAGAAACTGCAGCACTTGCTGGACCATCCTCGGCATGTGGAGA |
| | | TCCTGGGTTTCTCCGAGGCCAAAAGGAAAGAGTACTTCTTCAAGTACTTCTCTGATGAGGCCCAAGCCAGGGCAGCCTTCAGTCT |
| | | GATTCAGGAGAACGAGGTCCTCTTCACCATGTGCTTCATCCCCCTGGTCTGCTGGATCGTGTGCACTGGACTGAAACAGCAGATG |
| | | GAGAGTGGCAAGAGCCTTGCCCAGACATCCAAGACCACCACCGCGGTGTACGTCTTCTTCCTTTCCAGTTTGCTGCAGCCCCGGG |
| | | GAGGGAGCCAGGAGCACGGCCTCTGCGCCCACCTCTGGGGGCTCTGCTCTTTGGCTGCAGATGGAATCTGGAACCAGAAAATCCT |
| | | GTTTGAGGAGTCCGACCTCAGGAATCATGGACTGCAGAAGGCGGATGTGTCTGCTTTCCTGAGGATGAACCTGTTCCAAAAGGAA |
| | | GTGGACTGCGAGAAGTTCTACAGCTTCATCCACATGACTTTCCAGGAGTTCTTTGCCGCCATGTACTACCTGCTGGAAGAGGAAA |
| | | AGGAAGGAAGGACGAACGTTCCAGGGAGTCGTTTGAAGCTTCCCAGCCGAGACGTGACAGTCCTTCTGGAAAACTATGGCAAATT |
| | | CGAAAAGGGGTATTTGATTTTTGTTGTACGTTTCCTCTTTGGCCTGGTAAACCAGGAGAGGACCTCCTACTTGGAGAAGAAATTA |
| | | AGTTGCAAGATCTCTCAGCAAATCAGGCTGGAGCTGCTGAAATGGATTGAAGTGAAAGCCAAAGCTAAAAAGCTGCAGATCCAGC |
| | | CCAGCCAGCTGGAATTGTTCTACTGTTTGTACGAGATGCAGGAGGAGGACTTCGTGCAAAGGGCCATGGACTATTTCCCCAAGAT |
| | | TGAGATCAATCTCTCCACCAGAATGGACCACATGGTTTCTTCCTTTTGCATTGAGAACTGTCATCGGGTGGAGTCACTGTCCCTG |
| | | GGGTTTCTCCATAACATGCCCAAGGAGGAAGAGGAGGAGGAAAAGGAAGGCCGACACCTTGATATGGTGCAGTGTGTCCTCCCAA |
| | | GCTCCTCTCATGCTGCCTGTTCTCATGGATTGGTGAACAGCCACCTCACTTCCAGTTTTTGCCGGGGCCTCTTTTCAGTTCTGAG |
| | | CACCAGCCAGAGTCTAACTGAATTGGACCTCAGTGACAATTCTCTGGGGGACCCAGGGATGAGAGTGTTGTGTGAAACGCTCCAG |
| | | CATCCTGGCTGTAACATTCGGAGATTGTGGTTGGGGCGCTGTGGCCTCTCGCATGAGTGCTGCTTCGACATCTCCTTGGTCCTCA |
| | | GCAGCAACCAGAAGCTGGTGGAGCTGGACCTGAGTGACAACGCCCTCGGTGACTTCGGAATCAGACTTCTGTGTGTGGGACTGAA |
| | | GCACCTGTTGTGCAATCTGAAGAAGCTCTGGTTGGTGAATTCTGGCCTTACGTCAGTCTGTTGTTCAGCTTTGTCCTCGGTACTC |
| | | AGCACTAATCAGAATCTCACGCACCTTTACCTGCGAGGCAACACTCTCGGAGACAAGGGGATCAAACTACTCTGTGAGGGACTCT |
| | | TGCACCCCGACTGCAAGCTTCAGGTGTTGGAATTAGACAACTGCAACCTCACGTCACACTGCTGCTGGGATCTTTCCACACTTCT |
| | | GACCTCCAGCCAGAGCCTGCGAAAGCTGAGCCTGGGCAACAATGACCTGGGCGACCTGGGGGTCATGATGTTCTGTGAAGTGCTG |
| | | AAACAGCAGAGCTGCCTCCTGCAGAACCTGGGGTTGTCTGAAATGTATTTCAATTATGAGACAAAAAGTGCGTTAGAAACACTTC |
| | | AAGAAGAAAAGCCTGAGCTGACCGTCGTCTTTGAGCCTTCTTGGTAGGAGTGGAAACGGGGCTGCCAGACGCCAGTGTTCTCCGG |
| | | TCCCTCCAGCTGGGGGCCCTCAGGTGGAGAGAGCTGCGATCCATCCAGGCCAAGACCACAGCTCTGTGATCCTTCCGGTGGAGTG |
| | | TCGGAGAAGAGAGCTTGCCGACGATGCCTTCCTGTGCAGAGCTTGGGCATCTCCTTTACGCCAGGGTGAGGAAGACACCAGGACA |
| | | ATGACAGCATCGGGTGTTGTTGTCATCACAGCGCCTCAGTTAGAGGATGTTCCTCTTGGTGACCTCATGTAATTAGCTCATTCAA |
| | | TAAAGCACTTTCTTTATTTT |
| SEQ ID NO: | NM_001127462 | GTTCCTGAGGCTGGCATCTGGATGAGGAAACTGAAGTTGAGGAATAGTGAAGAGTTTGTCCAATGTCATAGCCCCGTAATCAACG |
| 534 | | GGACAAAAATTTTCTTGCTGATGGGTCAAGATGGCATCGTGAAGTGGTTGTTCACCGTAAACTGTAATACAATCCTGTTTATGGA |
| | | TTTGTTTGCATATTTTTCCCTCCATAGGGAAACCTTTCTTCCATGGCTCAGGACACACTCCTGGATCGAGCCAACAGGAGAACTT |
| | | TCTGGTAAGCATTTGGCTAACTTTTTTTTTTTTGAGATGGAGTCTTGCTGTGTCGCCTAGGCTGGAGTGCAGTGGCGTGATCTTG |
| | | GCTCACTGCAGCCTCCACTTCCCGGGTTCAATCAATTCTCCTACCTCAACTTCCTGAGTAGCTGGGATTACAGGCGCCCGCCACC |
| | | ACACCCGGCTCATTTTTGTACTTTTAGTAGAGACACAGTTTTGCCATGTTGGCCAGGCTGGTCTTGAATTCCTCAGCTCAGGTGA |
| | | TCTGCCTGCCTTGGCCTCTCAAAGTGCTGGGATTACAGGCGTGAGCCACTGTGCCCGGCCTTGGCTAACTTTTCAAAATTAAAGA |
| | | TTTTGACTTGTTACAGTCATGTGACATTTTTTTCTTTCTGTTTGCTGAGTTTTTGATAATTTATATCTCTCAAAGTGGAGACTTT |
| | | AAAAAAGACTCATCCGTGTGCCGTGTTCACTGCCTGGTATCTTAGTGTGGACCGAAGCCTAAGGACCCTGAAAACAGCTGCAGAT |
| | | GAAGATGGCAAGCACCCGCTGCAAGCTGGCCAGGTACCTGGAGGACCTGGAGGATGTGGACTTGAAGAAATTTAAGATGCACTTA |
| | | GAGGACTATCCTCCCCAGAAGGGCTGCATCCCCCTCCCGAGGGGTCAGACAGAGAAGGCAGACCATGTGGATCTAGCCACGCTAA |
| | | TGATCGACTTCAATGGGGAGGAGAAGGCGTGGGCCATGGCCGTGTGGATCTTCGCTGCGATCAACAGGAGAGACCTTTATGAGAA |
| | | AGCAAAAAGAGATGAGCCGAAGTGGGGTTCAGATAATGCACGTGTTTCGAATCCCACTGTGATATGCCAGGAAGACAGCATTGAA |
| | | GAGGAGTGGATGGGTTTACTGGAGTACCTTTCGAGAATCTCTATTTGTAAAATGAAGAAAGATTACCGTAAGAAGTACAGAAAGT |
| | | ACGTGAGAAGCAGATTCCAGTGCATTGAAGACAGGAATGCCCGTCTGGGTGAGAGTGTGAGCCTCAACAAACGCTACACACGACT |
| | | GCGTCTCATCAAGGAGCACCGGAGCCAGCAGGAGAGGGAGCAGGAGCTTCTGGCCATCGGCAAGACCAAGACGTGTGAGAGCCCC |
| | | GTGAGTCCCATTAAGATGGAGTTGCTGTTTGACCCCGATGATGAGCATTCTGAGCCTGTGCACACCGTGGTGTTCCAGGGGGCGG |
| | | CAGGGATTGGGAAAACAATCCTGGCCAGGAAGATGATGTTGGACTGGGCGTCGGGGACACTCTACCAAGACAGGTTTGACTATCT |
| | | GTTCTATATCCACTGTCGAGAGGTGAGCCTTGTGACACAGAGGAGCCTGGGGGACCTGATCATGAGCTGCTGCCCCGACCCAAAC |
| | | CCACCCATCCACAAGATCGTGAGAAAACCCTCCAGAATCCTCTTCCTCATGGACGGCTTCGATGAGCTGCAAGGTGCCTTTGACG |
| | | AGCACATAGGACCGCTCTGCACTGACTGGCAGAAGGCCGAGCGGGGAGACATTCTCCTGAGCAGCCTCATCAGAAAGAAGCTGCT |
| | | TCCCGAGGCCTCTCTGCTCATCACCACGAGACCTGTGGCCCTGGAGAAACTGCAGCACTTGCTGGACCATCCTCGGCATGTGGAG |
| | | ATCCTGGGTTTCTCCGAGGCCAAAAGGAAAGAGTACTTCTTCAAGTACTTCTCTGATGAGGCCCAAGCCAGGGCAGCCTTCAGTC |
| | | TGATTCAGGAGAACGAGGTCCTCTTCACCATGTGCTTCATCCCCCTGGTCTGCTGGATCGTGTGCACTGGACTGAAACAGCAGAT |
| | | GGAGAGTGGCAAGAGCCTTGCCCAGACATCCAAGACCACCACCGCGGTGTACGTCTTCTTCCTTTCCAGTTTGCTGCAGCCCCGG |
| | | GGAGGGAGCCAGGAGCACGGCCTCTGCGCCCACCTCTGGGGGCTCTGCTCTTTGGCTGCAGATGGAATCTGGAACCAGAAAATCC |
| | | TGTTTGAGGAGTCCGACCTCAGGAATCATGGACTGCAGAAGGCGGATGTGTCTGCTTTCCTGAGGATGAACCTGTTCCAAAAGGA |
| | | AGTGGACTGCGAGAAGTTCTACAGCTTCATCCACATGACTTTCCAGGAGTTCTTTGCCGCCATGTACTACCTGCTGGAAGAGGAA |
| | | AAGGAAGGAAGGACGAACGTTCCAGGGAGTCGTTTGAAGCTTCCCAGCCGAGACGTGACAGTCCTTCTGGAAAACTATGGCAAAT |
| | | TCGAAAAGGGGTATTTGATTTTTGTTGTACGTTTCCTCTTTGGCCTGGTAAACCAGGAGAGGACCTCCTACTTGGAGAAGAAATT |
| | | AAGTTGCAAGATCTCTCAGCAAATCAGGCTGGAGCTGCTGAAATGGATTGAAGTGAAAGCCAAAGCTAAAAAGCTGCAGATCCAG |
| | | CCCAGCCAGCTGGAATTGTTCTACTGTTTGTACGAGATGCAGGAGGAGGACTTCGTGCAAAGGGCCATGGACTATTTCCCCAAGA |
| | | TTGAGATCAATCTCTCCACCAGAATGGACCACATGGTTTCTTCCTTTTGCATTGAGAACTGTCATCGGGTGGAGTCACTGTCCCT |
| | | GGGGTTTCTCCATAACATGCCCAAGGAGGAAGAGGAGGAGGAAAAGGAAGGCCGACACCTTGATATGGTGCAGTGTGTCCTCCCA |
| | | AGCTCCTCTCATGCTGCCTGTTCTCATGGGTTGGGGCGCTGTGGCCTCTCGCATGAGTGCTGCTTCGACATCTCCTTGGTCCTCA |
| | | GCAGCAACCAGAAGCTGGTGGAGCTGGACCTGAGTGACAACGCCCTCGGTGACTTCGGAATCAGACTTCTGTGTGTGGGACTGAA |
| | | GCACCTGTTGTGCAATCTGAAGAAGCTCTGGTTGGTCAGCTGCTGCCTCACATCAGCATGTTGTCAGGATCTTGCATCAGTATTG |
| | | AGCACCAGCCATTCCCTGACCAGACTCTATGTGGGGGAGAATGCCTTGGGAGACTCAGGAGTCGCAATTTTATGTGAAAAAGCCA |
| | | AGAATCCACAGTGTAACCTGCAGAAACTGGGGTTGGTGAATTCTGGCCTTACGTCAGTCTGTTGTTCAGCTTTGTCCTCGGTACT |
| | | CAGCACTAATCAGAATCTCACGCACCTTTACCTGCGAGGCAACACTCTCGGAGACAAGGGGATCAAACTACTCTGTGAGGGACTC |
| | | TTGCACCCCGACTGCAAGCTTCAGGTGTTGGAATTAGACAACTGCAACCTCACGTCACACTGCTGCTGGGATCTTTCCACACTTC |
| | | TGACCTCCAGCCAGAGCCTGCGAAAGCTGAGCCTGGGCAACAATGACCTGGGCGACCTGGGGGTCATGATGTTCTGTGAAGTGCT |
| | | GAAACAGCAGAGCTGCCTCCTGCAGAACCTGGGGTTGTCTGAAATGTATTTCAATTATGAGACAAAAAGTGCGTTAGAAACACTT |
| | | CAAGAAGAAAAGCCTGAGCTGACCGTCGTCTTTGAGCCTTCTTGGTAGGAGTGGAAACGGGGCTGCCAGACGCCAGTGTTCTCCG |
| | | GTCCCTCCAGCTGGGGGCCCTCAGGTGGAGAGAGCTGCGATCCATCCAGGCCAAGACCACAGCTCTGTGATCCTTCCGGTGGAGT |
| | | GTCGGAGAAGAGAGCTTGCCGACGATGCCTTCCTGTGCAGAGCTTGGGCATCTCCTTTACGCCAGGGTGAGGAAGACACCAGGAC |
| | | AATGACAGCATCGGGTGTTGTTGTCATCACAGCGCCTCAGTTAGAGGATGTTCCTCTTGGTGACCTCATGTAATTAGCTCATTCA |
| | | ATAAAGCACTTTCTTTATTTT |
| SEQ ID NO: | | |
| 535-537 | | |
| SEQ ID NO: | Caspase-1 | Asn-Glu-Ala-Tyr-Val-His-Asp-Ala-Pro-Val-Arg-Ser-Leu-Asn |
| 538 | substrate | |
| SEQ ID NO: | NLRP3 protein | MKMASTRCKLARYLEDLEDVDLKKFKMHLEDYPPQKGCIPLPRGQTEKADHVDLATLMIDFNGEEKAWAMAVWIFA |
| 539 | corresponding | AINRRDLYEKAKRDEPKWGSDNARVSNPTVICQEDSIEEEWMGLLEYLSRISICKMKKDYRKKYRKYVRSRFQCIEDR |
| | to NM_004895.1 | NARLGESVSLNKRYTRLRLIKEHRSQQEREQELLAIGKTKTCESPVSPIKMELLFDPDDEHSEPVHTVVFQGAAGIGKTI |
| | | LARKMMLDWASGTLYQDRFDYLFYIHCREVSLVTQRSLGDLIMSCCPDPNPPIHKIVRKPSRILFLMDGFDELQGAFDE |
| | | HIGPLCTDWQKAERGDILLSSLIRKKLLPEASLLITTRPVALEKLQHLLDHPRHVEILGFSEAKRKEYFFKYFSDEAQAR |
| | | AAFSLIQENEVLFTMCFIPLVCWIVCTGLKQQMESGKSLAQTSKTTTAVYVFFLSSLLQPRGGSQEHGLCAHLWGLCSL |
| | | AADGIWNQKILFEESDLRNHGLQKADVSAFLRMNLFQKEVDCEKFYSFIHMTFQEFFAAMYYLLEEEKEGRTNVPGSR |
| | | LKLPSRDVTVLLENYGKFEKGYLIFVVRFLFGLVNQERTSYLEKKLSCKISQQIRLELLKWIEVKAKAKKLQIQPSQLEL |
| | | FYCLYEMQEEDFVQRAMDYFPKIEINLSTRMDHMVSSFCIENCHRVESLSLGFLHNMPKEEEEEEKEGRHLDMVQCVL |
| | | PSSSHAACSHGLVNSHLTSSFCRGLFSVLSTSQSLTELDLSDNSLGDPGMRVLCETLQHPGCNIRRLWLGRCGLSHECC |
| | | FDISLVLSSNQKLVELDLSDNALGDFGIRLLCVGLKHLLCNLKKLWLVSCCLTSACCQDLASVLSTSHSLTRLYVGENA |
| | | LGDSGVAILCEKAKNPQCNLQKLGLVNSGLTSVCCSALSSVLSTNQNLTHLYLRGNTLGDKGIKLLCEGLLHPDCKLQ |
| | | VLELDNCNLTSHCCWDLSTLLTSSQSLRKLSLGNNDLGDLGVMMFCEVLKQQSCLLQNLGLSEMYFNYETKSALETL |
| | | QEEKPELTVVFEPSW |
| SEQ ID NO: | NLRP3 inflammasome | GUGCAUUGAAGACAGGAAUTT |
| 540 | inhibitor | |
| SEQ ID NO: | | GGCTGTAACATTCGGAGATTG |
| 541 | | |
| SEQ ID NO: | | TCATCATTCCCGCTATCTTTC |
| 542 | | |
| SEQ ID NO: | | CCGTAAGAAGTACAGAAAGTA |
| 543 | | |
| SEQ ID NO: | | GAGACTCAGGAGTCGCAATTT |
| 544 | | |
| SEQ ID NO: | | CCTCATGTAATTAGCTCATTC |
| 545 | | |
| SEQ ID NO: | | GTGGATCTAGCCACGCTAATG |
| 546 | | |
| SEQ ID NO: | | CCACAGTGTAACCTGCAGAAA |
| 547 | | |
| SEQ ID NO: | | CCAGCCAGAGTCTAACTGAAT |
| 548 | | |
| SEQ ID NO: | | GCGTTAGAAACACTTCAAGAA |
| 549 | | |
| SEQ ID NO: | | GCTGGAATTGTTCTACTGTTT |
| 550 | | |
| SEQ ID NO: | | CCACATGACTTTCCAGGAGTT |
| 551 | | |
| SEQ ID NOs: | See Table 5B in | |
| 552-586 | specification | |
| SEQ ID NO: | mature miR-9 | UCU UUG GUU AUC U AG CUG UAU GA |
| 587 | (MIMAT0000441) | |
| SEQ ID NO: | hsa-miR-9-5p | UCUUUGGUUAUCUAGCUGUAUGA |
| 588 | | |
| SEQ ID NO: | miR-223 | TGGGGTATTTGACAAACTGACA |
| 589 | | |
| SEQ ID NO: | cbn-mir-233 | UCGCCCAUCCCGUUGUUCCAAUAUUCCAACAACAAGUGAUUAUUGAGCAAUGCGCAUGUGCGG |
| 590 | MI0024890 | |
| SEQ ID NO: | cbr-mir-233 | AAGCAUUUUUCUGUCCCGCGCAUCCCUUUGUUCCAAUAUUCAAACCAGUAGAAAGAUUAUUGAGCAAUGCGC |
| 591 | MI0000530 | AUGUGCGGGACAGAUUGAAUAGCUG |
| SEQ ID NO: | cel-mir-233 | AUAUAGCAUCUUUCUGUCUCGCCCAUCCCGUUGCUCCAAUAUUCUAACAACAAGUGAUUAUUGAGCAAUGCG |
| 592 | MI0000308 | CAUGUGCGGGAUAGACUGAUGGCUGC |
| SEQ ID NO: | crm-mir-233 | UGAAGCGUCUCUCUGUCCCGCUCAUCCUGUUGUUCCAAUAUUCCAACAGCCCAGUGAUUAUUGAGCAAUGCGC |
| 593 | MI0011059 | AUGUGCGGGACAGAUUGUAUGCUGCCAU |
| SEQ ID NO: | hsa-miR-22-5p | AGUUCUUCAGUGGCAAGCUUUA |
| 594 | MIMAT000449 | |
| SEQ ID NO: | hsa-mir-22 | GGCUGAGCCGCAGUAGUUCUUCAGUGGCAAGCUUUAUGUCCUGACCCAGCUAAAGCUGCCAGUUGAAGAACU |
| 595 | MI0000078 | GUUGCCCUCUGCC |
| SEQ ID NO: | mmu-miR-33-5p or | GUGCAUUGUAGUUGCAUUGCA |
| 596 | MIMAT0000667 | |
| SEQ ID NO: | mmu-mir-33 | CUGUGGUGCAUUGUAGUUGCAUUGCAUGUUCUGGCAAUACCUGUGCAAUGUUUCCACAGUGCAUCACGG |
| 597 | MI0000707 | |
| SEQ ID NO: | AIM2 (NP_004824.1) | MESKYKEILLLTGLDNITDEELDRFKFFLSDEFNIATGKLHTANRIQVATLMIQNAGAVSAVMKTIRIFQKLNYMLLAKRLQEEK |
| 598 | | EKVDKQYKSVTKPKPLSQAEMSPAASAAIRNDVAKQRAAPKVSPHVKPEQKQMVAQQESIREGFQKRCLPVMVLKAKKPFTFETQ |
| | | EGKQEMEHATVATEKEFFEVKVENTLLKDKEIPKRIIIIARYYRHSGFLEVNSASRVLDAESDQKVNVPLNIIRKAGETPKINTL |
| | | QTQPLGTIVNGLEVVQKVTEKKKNILFDLSDNTGKMEVLGVRNEDTMKCKEGDKVRLTEFTLSKNGEKLQLTSGVHSTIKVIKAK |
| | | KKT |
| SEQ ID NO: | | |
| 599 | | |
| SEQ ID NO: | Human Aim 2 | ATAGACATTTTCTTCTGTGGCTGCTAGTGAGAACCCAAACCAGCTCAGCCAATTAGAGCTCCAGTTGTCACTCCTACCCACACTG |
| 600 | (NM_004833.2) | GGCCTGGGGGTGAAGGGAAGTGTTTATTAGGGGTACATGTGAAGCCGTCCAGAAGTGTCAGAGTCTTTGTAGCTTTGAAAGTCAC |
| | | CTAGGTTATTTGGGCATGCTCTCCTGAGTCCTCTGCTAGTTAAGCTCTCTGAAAAGAAGGTGGCAGACCCGGTTTGCTGATCGCC |
| | | CCAGGGATCAGGAGGCTGATCCCAAAGTTGTCAGATGGAGAGTAAATACAAGGAGATACTCTTGCTAACAGGCCTGGATAACATC |
| | | ACTGATGAGGAACTGGATAGGTTTAAGTTCTTTCTTTCAGACGAGTTTAATATTGCCACAGGCAAACTACATACTGCAAACAGAA |
| | | TACAAGTAGCTACCTTGATGATTCAAAATGCTGGGGCGGTGTCTGCAGTGATGAAGACCATTCGTATTTTTCAGAAGTTGAATTA |
| | | TATGCTTTTGGCAAAACGTCTTCAGGAGGAGAAGGAGAAAGTTGATAAGCAATACAAATCGGTAACAAAACCAAAGCCACTAAGT |
| | | CAAGCTGAAATGAGTCCTGCTGCATCTGCAGCCATCAGAAATGATGTCGCAAAGCAACGTGCTGCACCAAAAGTCTCTCCTCATG |
| | | TTAAGCCTGAACAGAAACAGATGGTGGCCCAGCAGGAATCTATCAGAGAAGGGTTTCAGAAGCGCTGTTTGCCAGTTATGGTACT |
| | | GAAAGCAAAGAAGCCCTTCACGTTTGAGACCCAAGAAGGCAAGCAGGAGATGTTTCATGCTACAGTGGCTACAGAAAAGGAATTC |
| | | TTCTTTGTAAAAGTTTTTAATACACTGCTGAAAGATAAATTCATTCCAAAGAGAATAATTATAATAGCAAGATATTATCGGCACA |
| | | GTGGTTTCTTAGAGGTAAATAGCGCCTCACGTGTGTTAGATGCTGAATCTGACCAAAAGGTTAATGTCCCGCTGAACATTATCAG |
| | | AAAAGCTGGTGAAACCCCGAAGATCAACACGCTTCAAACTCAGCCCCTTGGAACAATTGTGAATGGTTTGTTTGTAGTCCAGAAG |
| | | GTAACAGAAAAGAAGAAAAACATATTATTTGACCTAAGTGACAACACTGGGAAAATGGAAGTACTGGGGGTTAGAAACGAGGACA |
| | | CAATGAAATGTAAGGAAGGAGATAAGGTTCGACTTACATTCTTCACACTGTCAAAAAATGGAGAAAAACTACAGCTGACATCTGG |
| | | AGTTCATAGCACCATAAAGGTTATTTAGGCCAAAAAAAAAACATAGAGAAGTAAAAAGGACCAATTCAAGCCAACTGGTCTAAGC |
| | | AGCATTTAATTGAAGAATATGTGATACAGCCTCTTCAATCAGATTGTAAGTTACCTGAAAGCTGCAGTTCACAGGCTCCTCTCTC |
| | | CACCAAATTAGGATAGAATAATTGCTGGATAAACAAATTCAGAATATCAACAGATGATCACAATAAACATCTGTTTCTCATTCAA |
| | | AAAAAAAAA |
| SEQ ID NO: | AIM2 inflammasome | CCCGAAGATCAACACGCTTCA |
| 601 | inhibitor | |
| SEQ ID NO: | A151 | TTAGGGTTAGGGTTAGGGTTAGGG |
| 602 | | |
| SEQ ID NO: | C151 | TTCAAATTCAAATTCAAATTCAAA |
| 603 | | |
| SEQ ID NO: | | TTAGGG |
| 604 | | |
| SEQ ID NOs: | See Table 5C in | |
| 605-610 | specification | |
| SEQ ID NO: | NM_0332923 | ATACTTTCAGTTTCAGTCACACAAGAAGGGAGGAGAGAAAAGCCATGGCCGACAAGGTCCTGAAGGAGAAGAGAAAGCTGTTTAT |
| 611 | | CCGTTCCATGGGTGAAGGTACAATAAATGGCTTACTGGATGAATTATTACAGACAAGGGTGCTGAACAAGGAAGAGATGGAGAAA |
| | | GTAAAACGTGAAAATGCTACAGTTATGGATAAGACCCGAGCTTTGATTGACTCCGTTATTCCGAAAGGGGCACAGGCATGCCAAA |
| | | TTTGCATCACATACATTTGTGAAGAAGACAGTTACCTGGCAGGGACGCTGGGACTCTCAGCAGATCAAACATCTGGAAATTACCT |
| | | TAATATGCAAGACTCTCAAGGAGTACTTTCTTCCTTTCCAGCTCCTCAGGCAGTGCAGGACAACCCAGCTATGCCCACATCCTCA |
| | | GGCTCAGAAGGGAATGTCAAGCTTTGCTCCCTAGAAGAAGCTCAAAGGATATGGAAACAAAAGTCGGCAGAGATTTATCCAATAA |
| | | TGGACAAGTCAAGCCGCACACGTCTTGCTCTCATTATCTGCAATGAAGAATTTGACAGTATTCCTAGAAGAACTGGAGCTGAGGT |
| | | TGACATCACAGGCATGACAATGCTGCTACAAAATCTGGGGTACAGCGTAGATGTGAAAAAAAATCTCACTGCTTCGGACATGACT |
| | | ACAGAGCTGGAGGCATTTGCACACCGCCCAGAGCACAAGACCTCTGACAGCACGTTCCTGGTGTTCATGTCTCATGGTATTCGGG |
| | | AAGGCATTTGTGGGAAGAAACACTCTGAGCAAGTCCCAGATATACTACAACTCAATGCAATCTTTAACATGTTGAATACCAAGAA |
| | | CTGCCCAAGTTTGAAGGACAAACCGAAGGTGATCATCATCCAGGCCTGCCGTGGTGACAGCCCTGGTGTGGTGTGGTTTAAAGAT |
| | | TCAGTAGGAGTTTCTGGAAACCTATCTTTACCAACTACAGAAGAGTTTGAGGATGATGCTATTAAGAAAGCCCACATAGAGAAGG |
| | | ATTTTATCGCTTTCTGCTCTTCCACACCAGATAATGTTTCTTGGAGACATCCCACAATGGGCTCTGTTTTTATTGGAAGACTCAT |
| | | TGAACATATGCAAGAATATGCCTGTTCCTGTGATGTGGAGGAAATTTTCCGCAAGGTTCGATTTTCATTTGAGCAGCCAGATGGT |
| | | AGAGCGCAGATGCCCACCACTGAAAGAGTGACTTTGACAAGATGTTTCTACCTCTTCCCAGGACATTAAAATAAGGAAACTGTAT |
| | | GAATGTCTGTGGGCAGGAAGTGAAGAGATCCTTCTGTAAAGGTTTTTGGAATTATGTCTGCTGAATAATAAACTTTTTTGAAATA |
| | | ATAAATCTGGTAGAAAAATG |
| SEQ ID NO: | NP_150634.1 human | MADKVLKEKRKLFIRSMGEGTINGLLDELLQTRVLNKEEMEKVKRENATVMDKTRALIDSVIPKGAQACQICITYICEEDSYLAG |
| 612 | caspaste-1 protein | TLGLSADQTSGNYLNMQDSQGVLSSFPAPQAVQDNPAMPTSSGSEGNVKLCSLEEAQRIWKQKSAEIYPIMDKSSRTRLALIICN |
| | | EEFDSIPRRTGAEVDITGMTMLLQNLGYSVDVKKNLTASDMTTELEAFAHRPEHKTSDSTFLVFMSHGIREGICGKKHSEQVPDI |
| | | LQLNAIFNMLNTKNCPSLKDKPKVIIIQACRGDSPGVVWFKDSVGVSGNLSLPTTEEFEDDAIKKAHIEKDFIAFCSSTPDNVSW |
| | | RHPTMGSVFIGRLIEHMQEYACSCDVEEIFRKVRFSFEQPDGRAQMPTTERVTLTRCFYLFPGH |
| SEQ ID NOs: | See Table 5E in | |
| 613-619 | specification | |
| SEQ ID NOs: | See Table 5F in | |
| 620-664 | specification | |
| SEQ ID NO: | AIM2 inflammasome | AAAGGTTAATGTCCCGCTGAA |
| 665 | inhibitor | |
| SEQ ID NOs: | See Table 5D in | |
| 666-803 | specification | |
| SEQ ID NO: 803 | RBS sequence | GCGCGCTCGCTCGCTC |
| SEQ ID NO: 804 | TRS sequence | GGTTGA |
| SEQ ID NO: 805 | TRS sequence | AGTT |
| SEQ ID NO: 806 | TRS sequence | GGTTGG |
| SEQ ID NO: 807 | TRS sequence | AGTTGG |
| SEQ ID NO: 808 | TRS sequence | AGTTGA |
| SEQ ID NO: 809 | Other motif | RRTTRR |
| SEQ ID NO: | Kaposi′s sarcoma- | MAAPRGRPKKDLTMEDLTAKISQLTVENRELRKALGSTADPRDRPLTATEKEAQLTATVGALSAAAAKKIEARVRTIF |
| 882 | associated | SKVVTQKQVDDALKGLSLRIDVCMSDGGTAKPPPGANNRRRRGASTTRAGVDD |
| | herpesvirus | |
| | protein ORF52 | |
| SEQ ID NO: | Cytoplasmic | LANAMAPPGMRLRSGRSTGAPLTRGSCRKRNRSPERCDLGDDLHLQPRRKHVADSVDGRECGPHTLPIPGSPTVFTSGLPAF |
| 883 | isoform (ORF73) | VSSPTLPVAPIPSPAPATPLPPPALLPPVTTSSSPIPPSHPVSPGTTDTHSPSPALPPTQSPESSQRPPLSSPTGRPDSSTPMRP |
| | | PPSQQTTPPHSPTTPPPEPPSKSSPDSLAPSTLRSLRKRRLSSPQGPSTLNPICQSPPVSPPRCDFANRSVYPPWATESPIYV |
| | | GSSSDGDTPPRQPPTSPISIGSSSPSEGSWGDDTAMLVLLAEIAEEASKNEKECSENNQAGEDNGDNEISKESQVDKDDN |
| | | DNKDDEEEQETDEEDEEDDEEDDEEDDEEDDEEDDEEDDEEDDEEEDEEEDEEEDEEEDEEEEEDEEDDDDEDNEDEE |
| | | DDEEEDKKEDEEDGGDGNKTLSIQSSQQQQEPQQQEPQQQEPQQQEPQQQEPQQQEPQQQEPQQQEPQQREPQQREP |
| | | QQREPQQREPQQREPQQREPQQREPQQREPQQREPQQREPQQREPQQREPQQQEPQQQEPQQQEPQQQEPQQQEPQQ |
| | | QEPQQQEPQQQEPQQQEPQQQEPQQQEPQQQEPQQQDEQQQDEQQQDEQQQDEQQQDEQQQDEQQQDEQQQDEQE |
| | | QQDEQQQDEQQQQDEQEQQEEQEQQEEQQQDEQQQDEQQQDEQQQDEQEQQDEQQQDEQQQQDEQEQQEEQEQQ |
| | | EEQEQQEEQEQQEEQEQELEEQEQELEEQEQELEEQEQELEEQEQELEEQEQELEEQEQELEEQEQELEEQEQELEEQE |
| | | QELEEQEQELEEQEQELEEQEQELEEQEQELEEQEQEQELEEVEEQEQEQEEQELEEVEEQEQEQEEQEEQELEEVEEQ |
| | | EEQELEEVEEQEEQELEEVEEQEQQGVEQQEQETVEEPIILHGSSSEDEMEVDYPVVSTHEQIASSPPGDNTPDDDPQPG |
| | | PSREYRYVLRTSPPHRPGVRMRRVPVTHPKKPHPRYQQPPVPYRQIDDCPAKARPQHIFYRRFLGKDGRRDPKCQWKF |
| | | AVIFWGNDPYGLKKLSQAFQFGGVKAGPVSCLPHPGPDQSPITYCVYVYCQNKDTSKKVQMARLAWEASHPLAGNL |
| | | QSSIVKFKKPLPLTQPGENQGPGDSPQEMT |
| SEQ ID NO: | Truncated | MAPPGMRLRSGRSTGAPLTRGSCRKRNRSPERCDLGDDLHLQPRRKHVADSVDGRECGPHTLPIPGSPTVFTSGLPAF |
| 884 | cytoplasmic | VSSPTLPVAPIPSPAPATPLPPPALLPPVTTSSSPIPPSHPVSPGTTDTHSPSPALPPTQSPESSQRPPLSSPTGRPDSSTP |
| | LANA isoform (ORF73) | |
| SEQ ID | miR-25 | GGCCAGTGTTGAGAGGCGGAGACTTGGGCAATTGCTGGACGCTGCCCTGGGCATTGCACTTGTCTCGGTCTGACA |
| NO: 885 | | GTGCCGGCC |
| SEQ ID | miR-93 | CTGGGGGCTCCAAAGTGCTGTTCGTGCAGGTAGTGTGATTACCCAACCTACTGCTGAGCTAGCACTTCCCGAGCC |
| NO: 886 | | CCCGG |
| SEQ ID NO: 887 | TLR9 inhibitory | 5′-CCTN(3-5)G(3-5)RR-3′ |
| | oligonucleotide | |
| SEQ ID NO: 888 | TLR9 inhibitory | TTAGGGn |
| | oligonucleotide | |
| SEQ ID NO: 889 | ODN-2088 | TCCTGGCGGGGAAGT |
| SEQ ID NO: 890 | ODN-2114 | TCCTGGAGGGGAAGT |
| SEQ ID NO: 891 | poly-G | GGGGGGGGGGGGGGGGGGGG |
| SEQ ID NO: 892 | ODN-A151 | TTAGGGTTAGGGTTAGGGTTAGGG |
| SEQ ID NO: 893 | G-ODN | CTCCTATTGGGGGTTTCCTAT |
| SEQ ID NO: 894 | IRS-869 | TCCTGGAGGGGTTGT |
| SEQ ID NO: 895 | INH-1 | CCTGGATGGGAATTCCCATCCAGG |
| SEQ ID NO: 896 | INH-4 | TTCCCATCCAGGCCTGGATGGGAA |
| SEQ ID NO: 897 | IRS-661 | TGCTTGCAAGCTTGCAAGCA |
| SEQ ID NO: 898 | 4024 | TCCTGGATGGGAAGT |
| SEQ ID NO: 899 | 4084F | CCTGGATGGGAA |
| SEQ ID NO: 900 | INH-13 | CTTACCGCTGCACCTGGATGGGAA |
| SEQ ID NO: 901 | INH-18 | CCTGGATGGGAACTTACCGCTGCA |
| SEQ ID NO: 902 | IRS-954 | TGCTCCTGGAGGGGTTGT |
| SEQ ID NO: 903 | AS1411 | GGTGGTGGTGGTTGTGGTGGTGGTGG |
| SEQ ID NO: 904 | Caspase-1 inhibitor | GAA GGC CCA UAU AGA GAA A |
| SEQ ID NO: 905 | AAV1 5′ WT-ITR | TTGCCCACTCCCTCTCTGCGCGCTCGCTCGCTCGGTGGGGCCTGCGGACCAAAGGTCCGCAGACGGCAGA |
| | (LEFT) | GGTCTCCTCTGCCGGCCCCACCGAGCGAGCGACGCGCGCAGAGAGGGAGTGGGCAACTCCATCACTAGGGTAA |
| SEQ ID NO: 906 | AAV1 3′ WT-ITR | TTACCCTAGTGATGGAGTTGCCCACTCCCTCTCTGCGCGCGTCGCTCGCTCGGTGGGGCCGGCAGAGGAGACCTC |
| | (RIGHT) | TGCCGTCTGCGGACCTTTGGTCCGCAGGCCCCACCGAGCGAGCGAGCGCGCAGAGAGGGAGTGGGCAA |
| SEQ ID NO: 907 | AAV2 5′ WT-ITR | CCTGCAGGCAGCTGCGCGCTCGCTCGCTCACTGAGGCCGCCCGGGCAAAGCCCGGGCGTCGGGCGACCTTTGGTC |
| | (LEFT) | GCCCGGCCTCAGTGAGCGAGCGAGCGCGCAGAGAGGGAGTGGCCAACTCCATCACTAGGGGTTCCT |
| SEQ ID NO: 908 | AAV2 3′ WT-ITR | AGGAACCCCTAGTGATGGAGTTGGCCACTCCCTCTCTGCGCGCTCGCTCGCTCACTGAGGCCGGGCGACCAAAGG |
| | (RIGHT) | TCGCCCGACGCCCGGGCTTTGCCCGGGCGGCCTCAGTGAGCGAGCGAGCGCGCAGCTGCCTGCAGG |
| SEQ ID NO: 909 | AAV3 5′ WT-ITR | TTGGCCACTCCCTCTATGCGCACTCGCTCGCTCGGTGGGGCCTGGCGACCAAAGGTCGCCAGACGGACGTGGGTT |
| | (LEFT) | TCCACGTCCGGCCCCACCGAGCGAGCGAGTGCGCATAGAGGGAGTGGCCAACTCCATCACTAGAGGTAT |
| SEQ ID NO: 910 | AAV3 3′ WT-ITR | ATACCTCTAGTGATGGAGTTGGCCACTCCCTCTATGCGCACTCGCTCGCTCGGTGGGGCCGGACGTGGAAACCCA |
| | (RIGHT) | CGTCCGTCTGGCGACCTTTGGTCGCCAGGCCCCACCGAGCGAGCGAGTGCGCATAGAGGGAGTGGCCAA |
| SEQ ID NO: 911 | AAV4 5′ WT-ITR | TTGGCCACTCCCTCTATGCGCGCTCGCTCACTCACTCGGCCCTGGAGACCAAAGGTCTCCAGACTGCCGGCCTCTG |
| | (LEFT) | GCCGGCAGGGCCGAGTGAGTGAGCGAGCGCGCATAGAGGGAGTGGCCAACT |
| SEQ ID NO: 912 | AAV4 3′ WT-ITR | AGTTGGCCACATTAGCTATGCGCGCTCGCTCACTCACTCGGCCCTGGAGACCAAAGGTCTCCAGACTGCCGGCCT |
| | (RIGHT) | CTGGCCGGCAGGGCCGAGTGAGTGAGCGAGCGCGCATAGAGGGAGTGGCCAA |
| SEQ ID NO: 913 | AAV5 5′ WT-ITR | TCCCCCCTGTCGCGTTCGCTCGCTCGCTGGCTCGTTTGGGGGGGCGACGGCCAGAGGGCCGTCGTCTGGCAGCTCT |
| | (LEFT) | TTGAGCTGCCACCCCCCCAAACGAGCCAGCGAGCGAGCGAACGCGACAGGGGGGAGAGTGCCACACTCTCAAGC |
| | | AAGGGGGTTTTGTAAG |
| SEQ ID NO: 914 | AAV5 3′ WT-ITR | CTTACAAAACCCCCTTGCTTGAGAGTGTGGCACTCTCCCCCCTGTCGCGTTCGCTCGCTCGCTGGCTCGTTTGGGG |
| | (RIGHT) | GGGTGGCAGCTCAAAGAGCTGCCAGACGACGGCCCTCTGGCCGTCGCCCCCCCAAACGAGCCAGCGAGCGAGCG |
| | | AACGCGACAGGGGGGA |
| SEQ ID NO: 915 | AAV6 5′ WT-ITR | TTGCCCACTCCCTCTAATGCGCGCTCGCTCGCTCGGTGGGGCCTGCGGACCAAAGGTCCGCAGACGGCAGAGGTC |
| | (LEFT) | |
| | | TCCTCTGCCGGCCCCACCGAGCGAGCGAGCGCGCATAGAGGGAGTGGGCAACTCCATCACTAGGGGTAT |
| SEQ ID NO: 916 | AAV6 3′ WT-ITR | ATACCCCTAGTGATGGAGTTGCCCACTCCCTCTATGCGCGCTCGCTCGCTCGGTGGGGCCGGCAGAGGAGACCTC |
| | (RIGHT) | TGCCGTCTGCGGACCTTTGGTCCGCAGGCCCCACCGAGCGAGCGAGCGCGCATTAGAGGGAGTGGGCAA |

| Name | Sequence | SEQ ID NO: |
|---|---|---|
| ITR-18 | AGGAACCCCTAGTGATGGAGTTGGCCACTCCCTCTCTGCGCGCTCGCTCGCTCACTGAGGCGCACGCCCGGGTTTCCCGGGCGGCCTCAGT | 917 |
| Right | GAGCGAGCGAGCGCGCAGCTGCCTGCAGG | |
| ITR-19 | AGGAACCCCTAGTGATGGAGTTGGCCACTCCCTCTCTGCGCGCTCGCTCGCTCACTGAGGCCGACGCCCGGGCTTTGCCCGGGCGGCCTCA | 918 |
| Right | GTGAGCGAGCGAGCGCGCAGCTGCCTGCAGG | |
| ITR-20 | AGGAACCCCTAGTGATGGAGTTGGCCACTCCCTCTCTGCGCGCTCGCTCGCTCACTGAGGCCGGGCGACCAAAGGTCGCCCGACGCCCGG | 919 |
| Right | GCGCCTCAGTGAGCGAGCGAGCGCGCAGCTGCCTGCAGG | |
| ITR-21 | AGGAACCCCTAGTGATGGAGTTGGCCACTCCCTCTCTGCGCGCTCGCTCGCTCACTGAGGCTTTGCCTCAGTGAGCGAGCGAGCGCGCAGC | 920 |
| Right | TGCCTGCAGG | |
| ITR-22 | AGGAACCCCTAGTGATGGAGTTGGCCACTCCCTCTCTGCGCGCTCGCTCGCTCACTGAGGCCGGGCGACAAAGTCGCCCGACGCCCGGGC | 921 |
| Right | TTTGCCCGGGCGGCCTCAGTGAGCGAGCGAGCGCGCAGCTGCCTGCAGG | |
| ITR-23 | AGGAACCCCTAGTGATGGAGTTGGCCACTCCCTCTCTGCGCGCTCGCTCGCTCACTGAGGCCGGGCGAAAATCGCCCGACGCCCGGGCTTT | 922 |
| Right | GCCCGGGCGGCCTCAGTGAGCGAGCGAGCGCGCAGCTGCCTGCAGG | |
| ITR-24 | AGGAACCCCTAGTGATGGAGTTGGCCACTCCCTCTCTGCGCGCTCGCTCGCTCACTGAGGCCGGGCGAAACGCCCGACGCCCGGGCTTTGC | 923 |
| Right | CCGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCGCTCGCTCACTGAGGCCGGGCAAAGCCCGACGCCCGGGCTTTGCCC | 924 |
| Right | GGGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCGCTCGCTCACTGAGGCCGGGCGACCAAAGGTCGCCCGACGCCCGG | 925 |
| Right | GTTTCCCGGGCGGCCTCAGTGAGCGAGCGAGCGCGCAGCTGCCTGCAGG | |
| ITR-27 | AGGAACCCCTAGTGATGGAGTTGGCCACTCCCTCTCTGCGCGCTCGCTCGCTCACTGAGGCCGGGCGACCAAAGGTCGCCCGACGCCCGG | 926 |
| Right | TTTCCGGGCGGCCTCAGTGAGCGAGCGAGCGCGCAGCTGCCTGCAGG | |
| ITR-28 | AGGAACCCCTAGTGATGGAGTTGGCCACTCCCTCTCTGCGCGCTCGCTCGCTCACTGAGGCCGGGCGACCAAAGGTCGCCCGACGCCCGTT | 927 |
| Right | TCGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCGCTCGCTCACTGAGGCCGGGCGACCAAAGGTCGCCCGACGCCCTTT | 928 |
| Right | GGGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCGCTCGCTCACTGAGGCCGGGCGACCAAAGGTCGCCCGACGCCTTTG | 929 |
| Right | GCGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCGCTCGCTCACTGAGGCCGGGCGACCAAAGGTCGCCCGACGCTTTGC | 930 |
| Right | GGCCTCAGTGAGCGAGCGAGCGCGCAGCTGCCTGCAGG | |
| ITR-32 | AGGAACCCCTAGTGATGGAGTTGGCCACTCCCTCTCTGCGCGCTCGCTCGCTCACTGAGGCCGGGCGACCAAAGGTCGCCCGACGTTTCGG | 931 |
| Right | CCTCAGTGAGCGAGCGAGCGCGCAGCTGCCTGCAGG | |
| ITR-49 | AGGAACCCCTAGTGATGGAGTTGGCCACTCCCTCTCTGCGCGCTCGCTCGCTCACTGAGGCCGGGCGACCAAAGGTCGCCCGACGGCCTC | 932 |
| Right | AGTGAGCGAGCGAGCGCGCAGCTGCCTGCAGG | |
| ITR-50 | AGGAACCCCTAGTGATGGAGTTGGCCACTCCCTCTCTGCGCGCTCGCTCGCTCACTGAGGCCGGGCGACCAAAGGTCGCCCGACGCCCGG | 933 |
| right | GCGGCCTCAGTGAGCGAGCGAGCGCGCAGCTGCCTGCAGG | |
| ITR-33 | CCTGCAGGCAGCTGCGCGCTCGCTCGCTCACTGAGGCCGCCCGGGAAACCCGGGCGTGCGCCTCAGTGAGCGAGCGAGCGCGCAGAGAG | 934 |
| Left | GGAGTGGCCAACTCCATCACTAGGGGTTCCT | |
| ITR-34 | CCTGCAGGCAGCTGCGCGCTCGCTCGCTCACTGAGGCCGTCGGGCGACCTTTGGTCGCCCGGCCTCAGTGAGCGAGCGAGCGCGCAGAG | 935 |
| Left | AGGGAGTGGCCAACTCCATCACTAGGGGTTCCT | |
| ITR-35 | CCTGCAGGCAGCTGCGCGCTCGCTCGCTCACTGAGGCCGCCCGGGCAAAGCCCGGGCGTCGGCCTCAGTGAGCGAGCGAGCGCGCAGAG | 936 |
| Left | AGGGAGTGGCCAACTCCATCACTAGGGGTTCCT | |
| ITR-36 | CCTGCAGGCAGCTGCGCGCTCGCTCGCTCACTGAGGCGCCCGGGCGTCGGGCGACCTTTGGTCGCCCGGCCTCAGTGAGCGAGCGAGCG | 937 |
| Left | CGCAGAGAGGGAGTGGCCAACTCCATCACTAGGGGTTCCT | |
| ITR-37 | CCTGCAGGCAGCTGCGCGCTCGCTCGCTCACTGAGGCAAAGCCTCAGTGAGCGAGCGAGCGCGCAGAGAGGGAGTGGCCAACTCCATCA | 938 |
| Left | CTAGGGGTTCCT | |
| ITR-38 | CCTGCAGGCAGCTGCGCGCTCGCTCGCTCACTGAGGCCGCCCGGGCAAAGCCCGGGCGTCGGGCGACTTTGTCGCCCGGCCTCAGTGAGC | 939 |
| Left | GAGCGAGCGCGCAGAGAGGGAGTGGCCAACTCCATCACTAGGGGTTCCT | |
| ITR-39 | CCTGCAGGCAGCTGCGCGCTCGCTCGCTCACTGAGGCCGCCCGGGCAAAGCCCGGGCGTCGGGCGATTTTCGCCCGGCCTCAGTGAGCGA | 940 |
| Left | GCGAGCGCGCAGAGAGGGAGTGGCCAACTCCATCACTAGGGGTTCCT | |
| ITR-40 | CCTGCAGGCAGCTGCGCGCTCGCTCGCTCACTGAGGCCGCCCGGGCAAAGCCCGGGCGTCGGGCGTTTCGCCCGGCCTCAGTGAGCGAGC | 941 |
| Left | GAGCGCGCAGAGAGGGAGTGGCCAACTCCATCACTAGGGGTTCCT | |
| ITR-41 | CCTGCAGGCAGCTGCGCGCTCGCTCGCTCACTGAGGCCGCCCGGGCAAAGCCCGGGCGTCGGGCTTTGCCCGGCCTCAGTGAGCGAGCG | 942 |
| Left | AGCGCGCAGAGAGGGAGTGGCCAACTCCATCACTAGGGGTTCCT | |
| ITR-42 | CCTGCAGGCAGCTGCGCGCTCGCTCGCTCACTGAGGCCGCCCGGGAAACCCGGGCGTCGGGCGACCTTTGGTCGCCCGGCCTCAGTGAGC | 943 |
| Left | GAGCGAGCGCGCAGAGAGGGAGTGGCCAACTCCATCACTAGGGGTTCCT | |
| ITR-43 | CCTGCAGGCAGCTGCGCGCTCGCTCGCTCACTGAGGCCGCCCGGAAACCGGGCGTCGGGCGACCTTTGGTCGCCCGGCCTCAGTGAGCGA | 944 |
| Left | GCGAGCGCGCAGAGAGGGAGTGGCCAACTCCATCACTAGGGGTTCCT | |
| ITR-44 | CCTGCAGGCAGCTGCGCGCTCGCTCGCTCACTGAGGCCGCCCGAAACGGGCGTCGGGCGACCTTTGGTCGCCCGGCCTCAGTGAGCGAGC | 945 |
| Left | GAGCGCGCAGAGAGGGAGTGGCCAACTCCATCACTAGGGGTTCCT | |
| ITR-45 | CCTGCAGGCAGCTGCGCGCTCGCTCGCTCACTGAGGCCGCCCAAAGGGCGTCGGGCGACCTTTGGTCGCCCGGCCTCAGTGAGCGAGCGA | 946 |
| Left | GCGCGCAGAGAGGGAGTGGCCAACTCCATCACTAGGGGTTCCT | |
| ITR-46 | CCTGCAGGCAGCTGCGCGCTCGCTCGCTCACTGAGGCCGCCAAAGGCGTCGGGCGACCTTTGGTCGCCCGGCCTCAGTGAGCGAGCGAGC | 947 |
| Left | GCGCAGAGAGGGAGTGGCCAACTCCATCACTAGGGGTTCCT | |
| ITR-47 | CCTGCAGGCAGCTGCGCGCTCGCTCGCTCACTGAGGCCGCAAAGCGTCGGGCGACCTTTGGTCGCCCGGCCTCAGTGAGCGAGCGAGCGC | 948 |
| Left | GCAGAGAGGGAGTGGCCAACTCCATCACTAGGGGTTCCT | |
| ITR-48 | CCTGCAGGCAGCTGCGCGCTCGCTCGCTCACTGAGGCCGAAACGTCGGGCGACCTTTGGTCGCCCGGCCTCAGTGAGCGAGCGAGCGCGC | 949 |
| Left | AGAGAGGGAGTGGCCAACTCCATCACTAGGGGTTCCT | |
| VH-02 | MDWTWRILFLVAAATGAHS | 950 |
| VK-A26 | MLPSQLIGFLLLWVPASRG | 951 |

### REFERENCES

1. Intracellular Nucleic Acid Detection in Autoimmunity. J. T. Crowl et al., Annu. Rev. Immunol. 2017, 35: 313-336
2. Innate Immune Recognition of DNA: a recent history. A. Dempsey and A. G. Bowie, Virology 2015 May, 0: 146-152
3. Cutting Edge: Antimalarial Drugs Inhibit IFN-β Production through Blockade of Cyclic GMP-AMP Synthase-DNA Interaction. J. An et al., J. Immunol. Mar. 27, 2015
4. Small molecule inhibition of cGAS reduces interferon expression in primary macrophages from autoimmune mice. J. Vincent et al., Nature Communications, 8:750
5. Discovery of PF-06928215 as a high affinity inhibitor of cGAS enabled by a novel fluorescence polarization assay. PLOS ONE. Sep. 21, 2017
6. The functional interactome of PYHIN immune regulators reveals IFIX is a sensor of viral DNA. B. A. Diner et al., Molecular Systems Biology, 2015, 11:787
7. Activation and Regulation of DNA-Driven Immune Responses. S. R. Paluden. Microbiology and Molecular Biology Reviews. 2015. 79(2): 225
8. Viral Evasion of DNA-stimulated innate immune responses. M. H. Christnesen and S. R. Paluden. Cellular and Molecular Immunology. 2017. 14:4-13
9. Sequence-specific activation of the DNA sensor cGAS by Y-form DNA structures as found in primary HIV-1 cDNA. A-M Herzner et al., 2015. Nature Immunology
10. RNA Polymerase III Detects Cytosolic DNA and Induces Type-I Interferons Through the RIG-I Pathway. Y-H Chiu et. al. 2009. Cell. 138(3): 576-591
11. Mi-R-25/93 mediates hypoxia-induced immunosuppression by repressing cGAS. M. Z. Wu et al. 2017. Nat. Cell Biol. 19(10):1286-1296
12. Cytoplasmic isoforms of Kaposi sarcoma herpesvirus LANA recruit and antagonize the innate immune DNA sensor cGAS. Zhang G. et al., Proc Natl Acad Sci USA. 2016 Feb. 23; 113 (8):E1034-43.
13. Kaposi's sarcoma-associated herpesvirus inhibitor of cGAS (KicGAS) Encoded by ORF52, is an Abundant Tegument protein and Is Required for Production of Infectious Progeny Viruses. W. Li, et al., J. Virol. 2016, 90(11): 5329

1. A method for inhibiting an immune response when a transgene is expressed in a cell, the method comprising:
administering to the cell a composition comprising a non-viral capsid-free DNA vector with covalently-closed ends (ceDNA vector), wherein the ceDNA vector comprises at least one heterologous nucleotide sequence operably positioned between two flanking inverted terminal repeat sequences (ITRs); and
administering to the cell at least one inhibitor of the immune response.

2. The method of claim 1, wherein the immune response is an innate immune response.

3. The method of claim 1, wherein the inhibitor of the immune response is an inhibitor of the innate immune response.

4. The method of claim 1, wherein the ceDNA vector further encodes the at least one inhibitor of the immune response.

5. The method of claim 1, wherein the inhibitor of the immune response is administered separately from the ceDNA vector.

6. The method of claim 1, wherein the inhibitor of the immune response is:
an inhibitor of the NLRP3 inflammasome, an inhibitor of the AIM2 inflammasome, or a caspase-1 inhibitor;
an inhibitor of cyclic GMP-AMP Synthase (cGAS);
an inhibitor of a toll like receptor (TLR); or
rapamycin or a rapamycin analog.

7. (canceled)

8. (canceled)

9. The method of claim 6, wherein the TLR inhibitor is a TLR9 inhibitor; and wherein the TLR9 inhibitor is:
a TLR9 inhibitory oligonucleotide;
an miRNA specific for TLR9;
an siRNA specific for TLR9; or
an antibody or antigen-binding fragment that binds TLR9.

10.-40. (canceled)

41. The method of claim 1, wherein:
the at least one heterologous nucleotide sequence is operably positioned between two flanking wild-type inverted terminal repeat sequences (WT-ITRs);
the at least one heterologous nucleotide sequence is operably positioned between two flanking mutant inverted terminal repeat sequences (mutant ITRs);
the at least one heterologous nucleotide sequence is operably positioned between two flanking inverted terminal repeat sequences, wherein one ITR is a WT-ITR and one ITR is a mutant ITR;
the ITRs are symmetric ITRs; or
the ITRs are asymmetric ITRs.

42.-45. (canceled)

46. The method of claim 1, wherein:
one or both of the ITRs are from a virus selected from a parvovirus, a dependovirus, and an adeno-associated virus (AAV);
the flanking ITRs are symmetric or asymmetric;
the flanking ITRs are symmetrical or substantially symmetrical;
the flanking ITRs are asymmetric;
one or both of the ITRs are wild type, or wherein both of the ITRs are wild-type;
the flanking ITRs are from different viral serotypes;
at least one of the ITRs is altered from a wild-type AAV ITR sequence by a deletion, addition, or substitution that affects the overall three-dimensional conformation of the ITR;
one or both of the ITRs are synthetic; or
one or both of the ITRs is not a wild type ITR, or wherein both of the ITRs are not wild-type.

47.-55. (canceled)

56. The method of claim 1, wherein:
one or both of the ITRs is modified by a deletion, insertion, and/or substitution in at least one of the ITR regions selected from A, A′, B, B′, C, C′, D, and D′;
the deletion, insertion, and/or substitution results in the deletion of all or part of a stem-loop structure normally formed by the A, A′, B, B′ C, or C′ regions;
one or both of the ITRs are modified by a deletion, insertion, and/or substitution that results in the deletion of all or part of a stem-loop structure normally formed by the B and B′ regions;
one or both of the ITRs are modified by a deletion, insertion, and/or substitution that results in the deletion of all or part of a stem-loop structure normally formed by the C and C′ regions;
one or both of the ITRs are modified by a deletion, insertion, and/or substitution that results in the deletion of part of a stem-loop structure normally formed by the B and B′ regions and/or part of a stem-loop structure normally formed by the C and C′ regions;
one or both of the ITRs comprise a single stem-loop structure in the region that normally comprises a first stem-loop structure formed by the B and B′ regions and a second stem-loop structure formed by the C and C′ regions;
one or both of the ITRs comprise a single stem and two loops in the region that normally comprises a first stem-loop structure formed by the B and B′ regions and a second stem-loop structure formed by the C and C′ regions; or
one or both of the ITRs comprise a single stem and a single loop in the region that normally comprises a first stem-loop structure formed by the B and B′ regions and a second stem-loop structure formed by the C and C′ regions.

57.-64. (canceled)

65. The method of claim 1, wherein the at least one heterologous nucleotide sequence is under the control of at least one regulatory switch.

66. (canceled)

67. (canceled)

68. The method of claim 1, wherein the ceDNA vector and/or the inhibitor of the immune response is in a nanocarrier that comprises a lipid nanoparticle (LNP).

69.-80. (canceled)

81. The method of claim 1, wherein the at least one heterologous nucleotide sequence, when transcribed or translated, corrects for an abnormal amount of an endogenous protein in a subject or corrects for an abnormal function or activity of an endogenous protein or pathway in a subject.

82. (canceled)

83. (canceled)

84. The method of claim 1, wherein the at least one heterologous nucleotide sequence encodes or comprises a nucleotide molecule selected from the group consisting of an RNAi, an siRNA, an miRNA, an lncRNA, and an antisense oligo- or polynucleotide;
wherein the at least one heterologous nucleotide sequence encodes a protein;
wherein the at least one heterologous nucleotide sequence encodes an agonist or an antagonist of an endogenous protein or pathway associated with the disease or disorder; or
wherein the at least one heterologous nucleotide sequence encodes an antibody.

85.-96. (canceled)

97. A host cell comprising a ceDNA expression construct that encodes the ceDNA vector produced by the method of claim 84.

98.-102. (canceled)

103. A method of producing a ceDNA vector, comprising: (a) incubating the host cell of claim 97 under conditions effective and for time sufficient to induce production of the ceDNA vector; and (b) isolating the ceDNA from the host cells.

104. A composition comprising a non-viral capsid-free DNA vector with covalently-closed ends (ceDNA vector), wherein the ceDNA vector comprises at least one heterologous nucleotide sequence operably positioned between two flanking inverted terminal repeat sequences (ITRs), wherein the ceDNA vector further encodes at least one inhibitor of the immune response.

105. (canceled)

106. The composition of claim 104, wherein the immune response is an innate immune response.

107.-149. (canceled)

150. A method of expressing an inhibitor of the immune response in a cell, the method comprising contacting the cell with the composition of claim 104.

151.-154. (canceled)

155. A cell comprising the composition of claim 104.

156. A kit comprising the composition of claim 104.
